(19) United States
(12) Patent Application Publication
(10) Pub. No.: US 2014/0243324 A1
(43) Pub. Date: Aug. 28, 2014

(54) USE OF HEMATOPOIETIC GROWTH FACTOR MIMETICS

(71) Applicants: Reid P. Bissonnette, San Diego, CA (US); Andrew R. Hudson, San Diego, CA (US); Keith B. Marschke, San Diego, CA (US); Deepa Rungta, San Diego, CA (US); Lin Zhi, San Diego, CA (US)

(72) Inventors: Reid P. Bissonnette, San Diego, CA (US); Andrew R. Hudson, San Diego, CA (US); Keith B. Marschke, San Diego, CA (US); Deepa Rungta, San Diego, CA (US); Lin Zhi, San Diego, CA (US)

(73) Assignee: LIGAND PHARMACEUTICALS INCORPORATED, San Diego, CA (US)

(21) Appl. No.: 13/885,148

(22) PCT Filed: Nov. 17, 2011
(86) PCT No.: PCT/US11/61247

§ 371 Date: Apr. 3, 2014

Related U.S. Application Data
(60) Provisional application No. 61/415,270, filed on Nov. 18, 2010.

(51) Int. Cl. C07C 243/38; C07D 207/327; C07D 233/61; C07D 209/08; C07D 491/113; C07D 207/27; C07D 235/18; C07D 413/04; C07D 401/10; C07D 413/12; C07D 317/68; C07D 295/155

(52) U.S. Cl. CPC C07C 243/38; C07D 317/68; C07D 207/327; C07D 233/61; C07D 209/08; C07D 295/155; C07D 207/27; C07D 235/18; C07D 413/04; C07D 401/10; C07D 413/12; C07D 491/113
USPC 514/228.2; 514/615; 514/466; 514/422; 514/397; 514/414; 514/231.8; 514/252.11; 514/234.5; 514/232.5; 514/230.5; 514/338

(57) ABSTRACT

The present invention relates to uses of small molecule mimetics of hematopoietic growth factors. In particular the present invention relates to uses of small molecule mimetics of erythropoietin.

### RELATED APPLICATIONS

This is a non-provisional application claiming the benefit of U.S. Provisional App. No. 61/415,270 filed on Nov. 18, 2010 entitled “USES OF SELECTIVE ERYTHROPOIETIN AGONISTS,” the contents of which is hereby incorporated by reference in its entirety.

### FIELD OF THE INVENTION

The present invention relates to uses of small molecule mimetics of hematopoietic growth factors. In particular, the present invention relates to uses of small molecule mimetics of erythropoietin.

### BACKGROUND

Hematopoietic growth factors (HGFs) include a family of biological molecules such as glycoproteins with important regulatory functions in the processes of proliferation, differentiation, and functional activation of hematopoietic progenitors and mature blood cells. HGF compounds can be potent regulators of blood cell proliferation and development in the bone marrow. They are able to augment hematopoiesis when bone marrow dysfunction exists. Recombinant DNA technology has made it possible to clone the genes responsible for many of these factors.

One example of an HGF is the glycoprotein hormone erythropoietin (EPO). EPO is an essential viability and growth factor for the erythrocytic progenitors. EPO acts primarily to rescue differentiating erythroid cells from cell death, i.e., apoptosis, to increase their survival. EPO is a member of the family of class I cytokines which fold into a compact globular structure consisting of 4 α-helical bundles, thus while its molecular mass is 30.4 kDa, EPO migrates with an apparent size of 34-38 kDa on SDS-polyacrylamide gels. The peptide core of 165 amino acids suffices for receptor-binding and in vitro stimulation of erythropoiesis, while the carbohydrate portion (40% of the total molecule) is required for the in vivo survival of the hormone. The 4 carbohydrate chains of EPO have been analyzed in detail. The 3 complex-type N-linked oligosaccharides at asparagines 24, 38 and 83 appear involved in stabilizing EPO in circulation.

During the fetal stage of mammalian development, EPO is mainly produced by hepatocytes. After birth, almost all circulating EPO originates from peritubular fibroblast-like cells located in the cortex of the kidneys. Transcription factors of the GATA-family may be important in the control of the time-specific and tissue-specific expression of the EPO gene. In adults, minor amounts of EPO mRNA are expressed in liver parenchyma, spleen, lung, testis and brain. In brain, EPO exerts neurotrophic and neuroprotective effects, which are separate from the action of circulating EPO on erythropoietic tissues. See e.g., Jelkmann, W., Internal Medicine Vol. 43, No. 8 (August 2004).

EPO binds to the homodimeric EPO receptor to act synergistically with several growth factors (e.g., SCF, GM-CSF, 1L-3, and IGF-1) to cause the maturation and proliferation of erythroid progenitor cells and their subsequent differentiation into red blood cells (RBCs). This process is mediated by a number of intracellular signaling pathways, for example, STAT5, Ras/MAPK and PI3K-GATA 1. Binding of EPO to the EPO receptor induces a conformational change in the homodimer leading to the phosphorylation and activation of EPO receptor-associated JAK2 kinase, which results in tyrosine phosphorylation of the EPO receptor as well as the activation of several downstream signaling pathways, including STAT5, STAT3, STAT1, Ras/MAPK, PI3K1AKT and GATA1.

Four forms of EPO have been approved for use in humans as erythropoiesis stimulating agents, and are currently marketed as treatments for varying types of anemia. All four forms of EPO are synthetic proteins produced by recombinant DNA technology in mammalian cells into which the human erythropoietin gene has been introduced, and include Epoetin alfa (marketed as Epogen® and Procrit® and identical to endogenous EPO), Epoetin beta (marketed only in Europe as NeoRecormon®), Darbepoetin Alfa (marketed as Aranesp®) and a methoxy polyethylene glycol-conjugated form of epoetin beta (marketed as Micera®). All are 165 amino acid glycoproteins and have the same biological effects and identical amino acid sequence as endogenous erythropoietin. Darbepoetin Alfa differs from endogenous EPO by containing two more N-linked oligosaccharide chains.

In addition to the aforementioned erythropoiesis stimulating agents, there are several other agents begin developed that represent reformulations or alternative chemical modifications, but which are still based on full-length EPO. Hematide™ is a synthetic dimeric PEG-modified peptide-based erythropoiesis stimulating agent immunologically distinct from EPO but which is believed to bind and activate EPO receptor signaling in a manner indistinguishable from EPO. CNTO-530 is a 58 kD EPO receptor agonist that links two EMP-1 (erythropoietin mimetic peptide-1) peptides to a proprietary MIMETIBODY (Ig Fc domain fusion protein) scaffold. It bears no homology to EPO, yet activates EPO receptor signaling in a manner similar to EPO.

### SUMMARY OF THE INVENTION

Some embodiments provided herein include use of a non-peptidyl small molecule mimetic of EPO in the preparation of a medicament for the treatment of a disorder associated with erythropoiesis by administering an initial effective amount of the mimetic of EPO, and a second effective amount of the mimetic of EPO, wherein the initial and the second effective amounts are substantially the same.

In some embodiments, the medicament is for the treatment of a disorder associated with erythropoiesis by further administering a third effective amount of the mimetic of EPO that is substantially the same as the initial and second effective amounts.

In some embodiments, the medicament is for the treatment of a disorder associated with erythropoiesis by further administering one or more subsequent effective amounts of the mimetic of EPO that is substantially the same as the initial, second, and third effective amounts.

In some embodiments, the initial and the second effective amounts are the same.

In some embodiments, the medicament is for the treatment of a disorder associated with erythropoiesis by administering a series of effective amounts that are substantially the same at least daily, at least weekly, or at least monthly.

Some embodiments include use of a non-peptidyl small molecule mimetic of EPO in the preparation of a medicament for the treatment of a disorder associated with erythropoiesis without titration of the amount of the mimetic of EPO administered.

In some embodiments, the medicament is for the treatment of a disorder associated with erythropoiesis by administering at least two successive effective amounts of the mimetic of EPO.

In some embodiments, the medicament is for the treatment of a disorder associated with erythropoiesis without titration over a period of time for administration that is daily, weekly, or monthly.

In some embodiments, the disorder associated with erythropoiesis comprises anemia.

In some embodiments, the anemia is associated with chronic kidney disease, cancer, palliative cancer therapy, chemotherapy, anemia of chronic disease, congestive heart failure, rheumatoid arthritis, COPD, chronic inflammatory conditions, or HIV infection.

In some embodiments, the mimetic of EPO is an EPO receptor partial agonist.

In some embodiments, the mimetic of EPO activates EPO receptor signaling through the PI3K-GATA1 pathway.

Some embodiments include a non-peptidyl small molecule mimetic of EPO for use in the treatment of a disorder associated with erythropoiesis by administering an initial effective amount of the mimetic of EPO, and a second effective amount of the mimetic of EPO, wherein the initial and the second effective amounts are substantially the same.

Some embodiments include use of a mimetic of EPO in the treatment of a disorder associated with erythropoiesis by further administering one or more subsequent effective amounts of the mimetic of EPO that is substantially the same as the initial and second effective amounts.

In some embodiments, the initial and the second effective amounts are the same.

Some embodiments include use of a mimetic of EPO in the treatment of a disorder associated with erythropoiesis by administering a series of effective amounts that are substantially the same at least daily, at least weekly, or at least monthly.

Some embodiments include a non-peptidyl small molecule mimetic of EPO for use in the treatment of a disorder associated with erythropoiesis without titration of the amount of the mimetic of EPO administered.

Some embodiments include use of a mimetic of EPO for use in the treatment of a disorder associated with erythropoiesis by administering at least two successive effective amounts of the mimetic of EPO.

Some embodiments include use of a mimetic of EPO for use in the treatment of a disorder associated with erythropoiesis without titration over a period of time for administration that is daily, weekly, or monthly.

In some embodiments, the disorder associated with erythropoiesis comprises anemia. In some embodiments, the anemia is associated with chronic kidney disease, cancer, palliative cancer therapy, chemotherapy, anemia of chronic disease, congestive heart failure, rheumatoid arthritis, COPD, chronic inflammatory conditions, or HIV infection.

In some embodiments, the mimetic of EPO is an EPO receptor partial agonist.

In some embodiments, the mimetic of EPO activates EPO receptor signaling through the PI3K-GATA1 pathway.

Some embodiments include a method of treating a disorder associated with erythropoiesis in a patient in need of such treatment, said method comprising: administering an initial effective amount of a non-peptidyl small molecule mimetic of EPO to the patient; and administering a second effective amount of the mimetic of EPO to the patient, wherein the initial and the second effective amounts are substantially the same.

Some embodiments include comprising administering a third effective amount of the mimetic of EPO to the patient that is substantially the same as the initial and second effective amounts.

Some embodiments include administering one or more subsequent effective amounts of the mimetic of EPO to the patient that is substantially the same as the initial, second, and third effective amounts.

In some embodiments, the initial and the second effective amounts are the same.

Some embodiments include administering a series of effective amounts that are substantially the same to the patient at least daily, at least weekly, or at least monthly.

Some embodiments include a method of treating a disorder associated with erythropoiesis to a patient in need of such treatment, said method comprising: administering an effective amount of a non-peptidyl small molecule mimetic of EPO to the patient, wherein the amount administered to the patient is not titrated over the period of time for administration.

In some embodiments, one or more subsequent effective amounts of the mimetic of EPO are administered to said patient.

In some embodiments, the period of time for administration includes administration daily, weekly, or monthly.

In some embodiments, the disorder associated with erythropoiesis comprises anemia. In some embodiments, the anemia is associated with chronic kidney disease, cancer, palliative cancer therapy, chemotherapy, anemia of chronic disease, congestive heart failure, rheumatoid arthritis, COPD, chronic inflammatory conditions, or HIV infection.

In some embodiments, the mimetic of EPO is an EPO receptor partial agonist.

In some embodiments, the mimetic of EPO activates EPO receptor signaling through the PI3K-GATA1 pathway.

In some of the foregoing methods and uses, the EPO mimetic comprises a compound of Formula (I) having the structure:

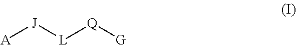

or a pharmaceutically acceptable salt, ester, stereoisomer, tautomer, or prodrug thereof.

In some of the foregoing methods and uses, the mimetic of EPO comprises a compound of Formula (II) having the structure:

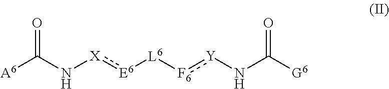

or a pharmaceutically acceptable salt, ester, stereoisomer, tautomer, or prodrug thereof.

In some of the foregoing methods and uses, the mimetic of EPO comprises a compound of Formula (III) having the structure:

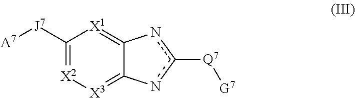

or a pharmaceutically acceptable salt, ester, stereoisomer, tautomer, or prodrug thereof.

In some of the foregoing methods and uses, the mimetic of EPO comprises a compound of Formula (IV) having the structure:

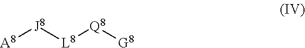

or a pharmaceutically acceptable salt, ester, stereoisomer, tautomer, or prodrug thereof.

In some of the foregoing methods and uses, the mimetic of EPO comprises a compound of Formula (V) having the structure:

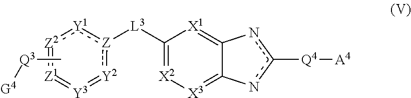

or a pharmaceutically acceptable salt, ester, stereoisomer, tautomer, or prodrug thereof.

In some of the foregoing methods and uses, the mimetic of EPO comprises a compound of Formula (VI) having the structure:

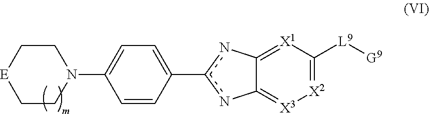

or a pharmaceutically acceptable salt, ester, stereoisomer, tautomer, or prodrug thereof.

In some of the foregoing methods and uses, the mimetic of EPO comprises a compound of Formula (VII) having the structure:

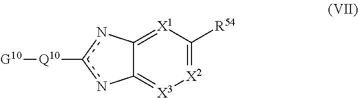

or a pharmaceutically acceptable salt, ester, stereoisomer, tautomer, or prodrug thereof.

In some of the foregoing methods and uses, the mimetic of EPO comprises a compound of Formula (IX) having the structure:

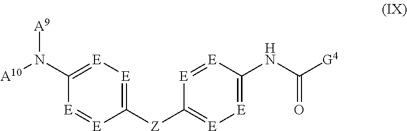

or a pharmaceutically acceptable salt thereof.

In some of the foregoing methods and uses, the mimetic of EPO comprises a compound of Formula (X) having the structure:

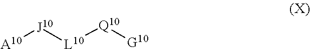

or a pharmaceutically acceptable salt, ester, stereoisomer, tautomer, or prodrug thereof.

### BRIEF DESCRIPTION OF THE DRAWINGS

FIG. 1 shows a schematic diagram of pathways related to the EPO receptor. Such pathways include the proteins JAK2, NFκB, IκB, MAPK, ERK, STAT5, PI3K, AKT, GATA1, and phosphorylated forms of such proteins.

FIG. 2 shows a diagram of changes in GATA1 and GATA2 activity as erythroid development progresses and cell proliferation decreases as cell differentiation increases. The erythroid development pathway includes the cells: EMP, BFU-E, CFU-E, Pro erythroblast, Basophilic erythroblast, Polychromatic erythroblast, Orthochromatic erythroblast, Reticulocyte, and Erythrocyte.

FIG. 3 shows a graph of percentage efficacy of experimental compounds relative to EPO vs. concentration of experimental compounds. (: recombinant human EPO (rHuEPO); ▴: compound 101; ▪: compound 102). Normal serum EPO concentration is about 0.01 U/ml (range: 0.006-0.032 U/ml).

FIG. 4 shows a graph of percentage differentiation of CD235a+ cells relative to number of cells treated with 2.0 U/ml recombinant human EPO (rHuEPO) for cells treated with various experimental compounds.

FIG. 5A shows a graph of phosphorylation of the EPO receptor after 15 minutes or 45 minutes treatment with 10 nM Compound 102, 100 nM Compound 102, or control (vehicle). FIG. 5B shows a graph of fold-stimulation of PI3K after 60 minutes treatment with EPO, Compound 101, or control (vehicle). FIG. 5C shows fold stimulation of GATA1 after 60 minutes treatment with EPO, Compound 101, or control (vehicle).

FIG. 6A shows the results of an electrophoretic mobility shift assay for GATA1 in UT7 cells after 30 minute treatments with test compounds. FIG. 6B shows a graph of binding intensity for GATA1 in UT7 cells after 30 minute treatments with test compounds.

FIG. 7 shows a graph of relative cell viability of an EPO dependent cell line treated with EPO receptor siRNA, GATA1 siRNA, or no siRNA. Cells were further treated with 1 U/ml EPO, 30 nM Compound 101, or control (vehicle).

### DETAILED DESCRIPTION

The present invention relates to methods and uses of certain compounds and compositions with activity that modulate activity of the EPO receptor. In some embodiments, certain compounds and compositions that modulate activity of the EPO also include erythropoiesis stimulating activity. In some embodiments, compounds and compositions that modulate activity of the EPO receptor can include non-peptidyl small molecule mimetics of EPO.

EPO acts on the homodimeric EPO receptor to stimulate proliferation of erythroid progenitor cells and induce their survival and differentiation into red blood cells. Various recombinant human EPO derivatives, also known as erythropoiesis-stimulating agents, are marketed or in clinical development for the treatment of anemia due to renal failure or cancer chemotherapy. However, treatment with erythropoiesis-stimulating agent is associated with an increased risk of adverse cardiovascular complications in patients with kidney disease, and may be related to an increase in mortality in cancer patients, when it is used to increase hemoglobin levels above 13.0 g/dl (Rizzo J D, et al. 2008 Use of epoetin and darbepoetin in patients with cancer: 2007 American Society of Clinical Oncology/American Society of Hematology clinical practice guidelines update. J Clin Oncol 26:132-149). Moreover, such undesirable effects are known to increase the risk of heart attack, heart failure, stroke, or blood clots in patients. Further shortcomings related to treatment with erythropoiesis-stimulating agents include relatively high cost of manufacture, administration though parenteral route, i.e. injection, and potential immunogenicity of erythropoiesis-stimulating agents.

Some embodiments of the present invention relate to the use of a non-peptidyl small molecules that selectively activate EPO receptor function. It has been discovered that some selective agonists display an efficacy partial to the maximal effect induced by EPO. Accordingly, some compounds and compositions provided herein are most advantageous in at least the lack excessive erythropoietic stimulation that may contribute to the adverse effects of erythropoiesis-stimulating agents. In addition, some compounds and compositions provided herein include further advantages over conventional erythropoiesis-stimulating agents. For example, some compounds and compositions provided herein are less likely to stimulate the production of neutralizing antibodies, can be orally administrable, thus eliminating the need for injections, have longer serum half-life, have an increased bioavailability, and/or can be produced at a lower cost than erythropoiesis-stimulating agents.

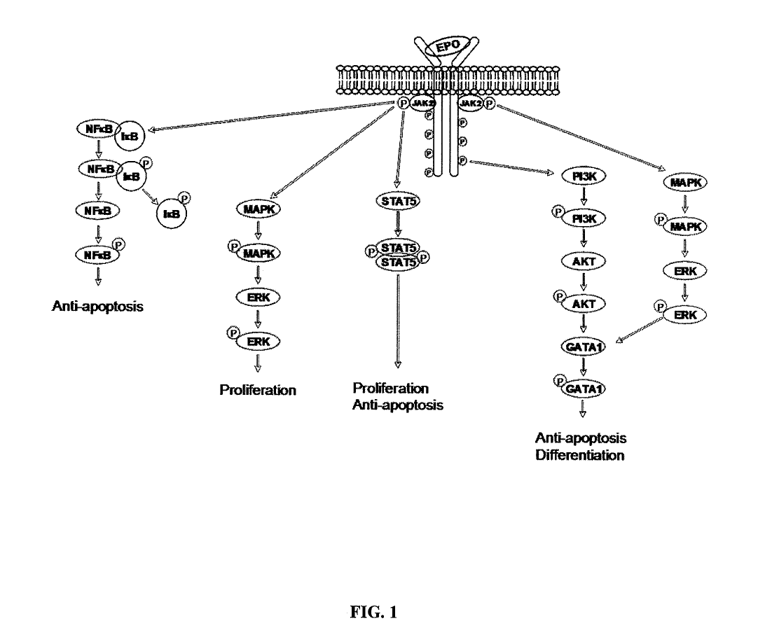

The EPO receptor is a 59 kDa homodimeric protein and is a member of the cytokine receptor family. Binding of EPO to the EPO receptor induces a conformational change resulting in the autophosphorylation of kinases such as Jak2, and activation of several intracellular signaling pathways (FIG. 1). The intracellular signaling pathways include Iκβ/NFκβ, Ras/MAPK/ERK, STAT5, P13/AKT/GATA1 pathways associated with cellular functions such as anti-apoptosis, proliferation, and differentiation of erythroid progenitor cells into red blood cells (Richmond T D, et al. 2005 Turning cells red: signal transduction mediated by erythropoietin. Trends Cell Biol 15:146-155).

In some embodiments, the compositions and methods provided herein can be used to treat disorders associated with anemia, such as chronic kidney disease, congestive heart failure, rheumatoid arthritis, and other chronic inflammatory diseases. In addition, anemia is a frequent complication in patients with cancer, oftentimes due to impaired EPO production, bone marrow response, iron availability, and red blood cell survival (Adamson J W. 2008 The anemia of inflammation/malignancy: mechanisms and management. Hematology Am Soc Hematol Educ Program. 2008:159-65). Anemia may also be associated with chemotherapy, especially when platinum-based therapy is used. However, despite evidence that some patients benefit from treatment with erythropoiesis-stimulating agent, some studies have also shown that erythropoiesis-stimulating agent can increase the risk of tumor growth and shorten survival in anemic cancer patients (Rizzo J D, et al. 2008 Use of epoetin and darbepoetin in patients with cancer: 2007 American Society of Clinical Oncology/American Society of Hematology clinical practice guidelines update. J Clin Oncol 26:132-149)

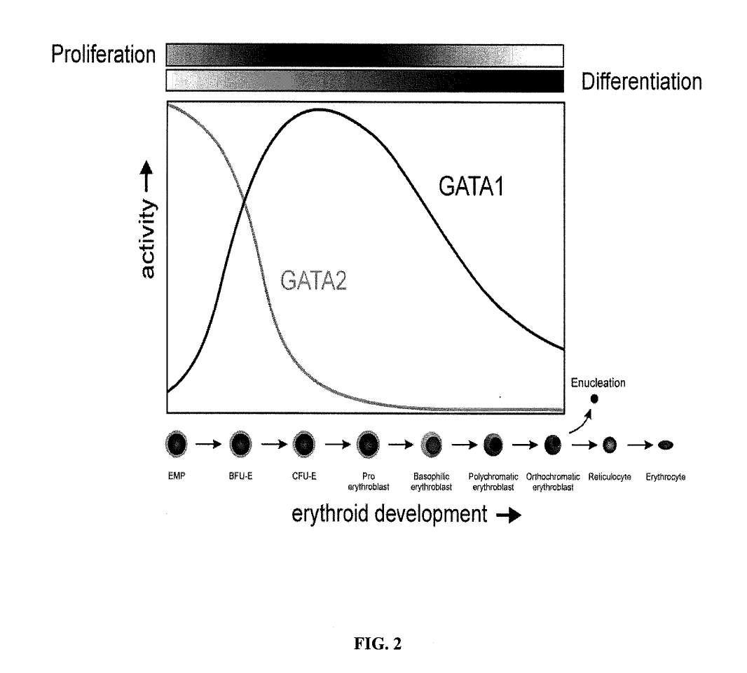

Additional advantages associated with some of the compounds and compositions provided herein relate to the selective activation of particular pathways. Some of the compounds and compositions provided herein selectively signal through one or more of the intracellular signaling pathways modulated by the EPO receptor. For example, some of the compounds and compositions provided herein selectively signal through the P13/AKT/GATA1 that is associated with differentiation of bone marrow hematopoietic cells (BM-HCs) into erythrocytes. The survival of erythroid progenitor cells and their terminal differentiation into RBCs is associated with EPO/EPO receptor interactions and GATA1 transcription factor activity (FIG. 2). The erythroid development pathway includes cells at various stages such as: EMP, BFU-E, CFU-E, pro-erythroblast, basophilic erythroblast, polychromatic erythroblast, orthochromatic erythroblast, reticulocyte, and erythrocyte. An increase in GATA1 cellular levels causes erythroid cells to develop the erythrocyte phenotype (e.g., expression of hemoglobin), and triggers the transition of rapidly proliferating erythroid progenitor cells to mature erythroid cells (Ferreira R, et al. 2005 GATA1 function, a paradigm for transcription factors in hematopoiesis. Mol Cell Biol 25:1215-27). The selective activation of particular pathways and not other pathways may reduce the risk of tumor growth in cancer patients The risk of tumor growth may be increased and survival may be shortened in cancer patients when particular pathways are stimulated.

Data provided herein demonstrate certain compounds disclosed herein include selective EPOR agonists that unlike erythropoiesis-stimulating agents, selectively activates the EPO receptor/PI3K/GATA1 signal transduction pathway resulting in survival and differentiation of BM-HCs into erythrocytes. The selective agonists display an efficacy partial to the maximal effect induced by EPO, and lack excessive erythropoietic stimulation that may possibly contribute to the adverse effects of erythropoiesis-stimulating agents.

Administration of erythropoiesis-stimulating agents generally require a patient to undergo a drug titration procedure in order to balance therapeutic effects with undesirable side effects. Drug titration can include administering an initial dose of a drug comprising relatively small dosage, then monitoring the patient's side effects, and subsequently increasing the dosage of the drug until the targeted benefits are achieved or until the patient's side effects meet or exceed a desired threshold. The dosage is increased until the patient's side effects increase to the point that they are judged to warrant no further increase in dosage or that the patient reaches a maximum recommended dosage. Drug titration techniques require face-to-face interaction with medical personnel, which consumes resources and is inconvenient for the patient. It will be understood that certain compounds and compositions provided herein with partial EPO receptor modulatory activity may not require a drug titration procedure. Accordingly, some embodiments of the present invention include administering certain compounds and compositions provided herein without a drug titration procedure.

### DEFINITIONS

Unless specific definitions are provided, the nomenclatures utilized in connection with, and the laboratory procedures and techniques of, analytical chemistry, synthetic organic chemistry, and medicinal and pharmaceutical chemistry described herein are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, pharmaceutical preparation, formulation, and delivery, and treatment of patients. Standard techniques may be used for recombinant DNA, oligonucleotide synthesis, and tissue culture and transformation (e.g., electroporation, lipofection). Reactions and purification techniques may be performed e.g., using kits according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference in its entirety for any purpose.

As used herein, the following terms are defined with the following meanings, unless expressly stated otherwise.

The term “selective binding compound” refers to a compound that selectively binds to any portion of one or more target.

The term “selective HGF receptor binding compound” refers to a compound that selectively binds to any portion of a HGF receptor.

The term “selective EPO receptor binding compound” refers to a compound that selectively binds to any portion of a EPO receptor.

The term “selectively binds” refers to the ability of a selective binding compound to bind to a target receptor with greater affinity than it binds to a non-target receptor. In certain embodiments, selective binding refers to binding to a target with an affinity that is at least 10, 50, 100, 250, 500, or 1000 times greater than the affinity for a non-target.

The term “target receptor” refers to a receptor or a portion of a receptor capable of being bound by a selective binding compound. In certain embodiments, a target receptor is a HGF receptor. In some embodiments, a target receptor is an EPO receptor.

The term “modulator” refers to a compound that alters an activity. For example, a modulator may cause an increase or decrease in the magnitude of a certain activity compared to the magnitude of the activity in the absence of the modulator. In certain embodiments, a modulator is an inhibitor, which decreases the magnitude of one or more activities. In certain embodiments, an inhibitor completely prevents one or more biological activities. In certain embodiments, a modulator is an activator, which increases the magnitude of at least one activity. In certain embodiments the presence of a modulator results in an activity that does not occur in the absence of the modulator.

The term “selective modulator” refers to a compound that selectively modulates a target activity.

The term “selective HGF modulator” refers to a compound that selectively modulates at least one HGF activity. The term selective HGF modulator includes, but is not limited to “HGF mimic” which refers to a compound, the presence of which results in at least one HGF activity.

The term “selective EPO receptor modulator” refers to a compound that selectively modulates at least one EPO receptor activity. The term “selective EPO receptor modulator” includes, but is not limited to “EPO mimic” which refers to a compound, the presence of which results in at least one EPO receptor activity.

The term “selectively modulates” refers to the ability of a selective modulator to modulate a target activity to a greater extent than it modulates a non-target activity.

The term “target activity” refers to a biological activity capable of being modulated by a selective modulator. Certain exemplary target activities include, but are not limited to, binding affinity, signal transduction, enzymatic activity, the proliferation and/or differentiation of progenitor cells, generation of platelets, and alleviation of symptoms of a disease or condition.

The term “HGF activity” refers to a biological activity that results, either directly or indirectly from the presence of HGF. Exemplary HGF activities include, but are not limited to, proliferation and or differentiation of progenitor cells to produce platelets; hematopoiesis; growth and/or development of glial cells; repair of nerve cells; and alleviation of thrombocytopenia. In some embodiments, an example of HGF activity is EPO receptor activity.

The term “receptor mediated activity” refers to any biological activity that results, either directly or indirectly, from binding of a ligand to a receptor.

The term “agonist” refers to a compound, the presence of which results in a biological activity of a receptor that is the same as the biological activity resulting from the presence of a naturally occurring ligand for the receptor.

The term “partial agonist” refers to a compound, the presence of which results in a biological activity of a receptor that is of the same type as that resulting from the presence of a naturally occurring ligand for the receptor, but of a lower magnitude.

The term “partial EPO receptor agonist” refers to a compound, the presence of which results in a biological activity of EPO receptor that is of the same type as that resulting from the presence of naturally occurring EPO, but which is less than the maximum effect induced by EPO.

The term “antagonist” refers to a compound, the presence of which results in a decrease in the magnitude of a biological activity of a receptor. In certain embodiments, the presence of an antagonist results in complete inhibition of a biological activity of a receptor.

The term “alkyl” refers to a branched or unbranched fully saturated acyclic aliphatic hydrocarbon group. An alkyl may be branched or straight chain. Alkyls may be substituted or unsubstituted. Alkyls include, but are not limited to, methyl, ethyl, propyl, isopropyl, butyl, isobutyl, tertiary butyl, pentyl, hexyl, and the like, each of which may be optionally substituted.

In certain embodiments, an alkyl comprises 1 to 20 carbon atoms (whenever it appears herein, a numerical range such as “1 to 20” refers to each integer in the given range; e.g., “1 to 20 carbon atoms” means that an alkyl group may comprise only 1 carbon atom, 2 carbon atoms, 3 carbon atoms, etc., up to and including 20 carbon atoms, although the term “alkyl” also includes instances where no numerical range of carbon atoms is designated). An alkyl may be designated as “C₁-C₆ alkyl” or similar designations. By way of example only, “C₁-C₄ alkyl” indicates an alkyl having one, two, three, or four carbon atoms, e.g., the alkyl is selected from methyl, ethyl, propyl, iso-propyl, butyl, iso-butyl, sec-butyl, and tert-butyl.

The term “alkenyl” used herein refers to a monovalent straight or branched chain aliphatic hydrocarbon radical of from two to twenty carbon atoms containing at least one carbon-carbon double bond including, but not limited to, 1-propenyl, 2-propenyl, 2-methyl-1-propenyl, 1-butenyl, 2-butenyl, and the like. In certain embodiments, an alkenyl comprises 2 to 20 carbon atoms (whenever it appears herein, a numerical range such as “2 to 20” refers to each integer in the given range; e.g., “2 to 20 carbon atoms” means that an alkenyl group may comprise only 2 carbon atoms, 3 carbon atoms, etc., up to and including 20 carbon atoms, although the term “alkenyl” also includes instances where no numerical range of carbon atoms is designated). An alkenyl may be designated as “C₂-C₆ alkenyl” or similar designations. By way of example only, “C₂-C₄ alkenyl” indicates an alkenyl having two, three, or four carbon atoms, e.g., the alkenyl is selected from ethenyl, propenyl, and butenyl.

The term “alkynyl” used herein refers to a monovalent straight or branched chain aliphatic hydrocarbon radical of from two to twenty carbon atoms containing at least one carbon-carbon triple bond including, but not limited to, 1-propynyl, 1-butynyl, 2-butynyl, and the like. In certain embodiments, an alkynyl comprises 2 to 20 carbon atoms (whenever it appears herein, a numerical range such as “2 to 20” refers to each integer in the given range; e.g., “2 to 20 carbon atoms” means that an alkynyl group may comprise only 2 carbon atoms, 3 carbon atoms, etc., up to and including 20 carbon atoms, although the term “alkynyl” also includes instances where no numerical range of carbon atoms is designated). An alkynyl may be designated as “C₂-C₆ alkynyl” or similar designations. By way of example only, “C₂-C₄ alkynyl” indicates an alkenyl having two, three, or four carbon atoms, e.g., the alkenyl is selected from ethynyl, propynyl, and butynyl.

The term “cycloalkyl” used herein refers to saturated aliphatic ring system radical having three to twenty carbon atoms. A cycloalkyl refers to monocyclic and polycyclic saturated aliphatic ring system including, but not limited to, cyclopropyl, cyclopentyl, cyclohexyl, cycloheptyl, bicyclo[4.4.0]decanyl, bicyclo[2.2.1]heptanyl, adamantyl, norbornyl, and the like. In certain embodiments, a cycloalkyl comprises 3 to 20 carbon atoms (whenever it appears herein, a numerical range such as “3 to 20” refers to each integer in the given range; e.g., “3 to 20 carbon atoms” means that a cycloalkyl group may comprise only 3 carbon atoms, etc., up to and including 20 carbon atoms, although the term “cycloalkyl” also includes instances where no numerical range of carbon atoms is designated). A cycloalkyl may be designated as “C₃-C₇ cycloalkyl” or similar designations. By way of example only, “C₃-C₆ cycloalkyl” indicates an alkenyl having two, three, four, five or six carbon atoms, e.g., the cycloalkyl is selected from cyclopropyl, cyclobutyl, cyclopentyl, and cyclohexyl.

The term “cycloalkenyl” used herein refers to aliphatic ring system radical having three to twenty carbon atoms having at least one carbon-carbon double bond in the ring. A cycloalkenyl refers to monocyclic and polycyclic unsaturated aliphatic ring system including, but are not limited to, cyclopropenyl, cyclopentenyl, cyclohexenyl, cycloheptenyl, bicyclo[3.1.0]hexyl, norbornylenyl, 1,1′-bicyclopentenyl, and the like. In certain embodiments, a cycloalkenyl comprises 3 to 20 carbon atoms (whenever it appears herein, a numerical range such as “3 to 20” refers to each integer in the given range; e.g., “3 to 20 carbon atoms” means that a cycloalkenyl group may comprise only 3 carbon atoms, etc., up to and including 20 carbon atoms, although the term “cycloalkenyl” also includes instances where no numerical range of carbon atoms is designated). A cycloalkenyl may be designated as “C₃-C₇ cycloalkenyl” or similar designations. By way of example only, “C₃-C₆ cycloalkenyl” indicates an alkenyl having two, three, four, five or six carbon atoms, e.g., the cycloalkyl is selected from cyclopropenyl, cyclobutenyl, cyclopentenyl, and cyclohexenyl.

The term “haloalkyl” refers to an alkyl in which at least one hydrogen atom is replaced with a halogen atom. In certain of the embodiments in which two or more hydrogen atom are replaced with halogen atoms, the halogen atoms are all the same as one another. In certain of such embodiments, the halogen atoms are not all the same as one another.

The term “heteroalkyl” refers to a group comprising an alkyl and one or more heteroatoms. Certain heteroalkyls are acylalkyls, in which the one or more heteroatoms are within an alkyl chain. Examples of heteroalkyls include, but are not limited to, CH₃C(═O)CH₂—, CH₃C(═O)CH₂CH₂—, CH₃CH₂C(═O)CH₂CH₂—, CH₃C(═O)CH₂CH₂CH₂—, CH₃OCH₂CH₂—, CH₃NHCH₂—, CH₃NHC(═O)CH₂—, and the like.

The term “alkoxy” used herein refers to straight or branched chain alkyl radical covalently bonded to the parent molecule through an —O— linkage. Examples of alkoxy groups include, but are not limited to, methoxy, ethoxy, propoxy, isopropoxy, butoxy, n-butoxy, sec-butoxy, t-butoxy and the like. An alkoxy may be designated as “C₁-C₆ alkoxy” or similar designations. By way of example only, “C₁-C₄ alkoxy” indicates an alkyl having one, two, three, or four carbon atoms, e.g., the alkoxy is selected from methoxy, ethoxy, propoxy, iso-propoxy, butoxy, iso-butoxy, sec-butoxy, and tert-butoxy.

The term “olefin” refers to a C═C bond.

The term “alkylideneamino” used herein refers to a moiety of from one to twenty carbon atoms containing at least one carbon-nitrogen double bond where the moiety is connected to the main group through the nitrogen, including, but not limited to, methylideneamino, ethylideneamino, methylethylideneamino, propylideneamino, 1-methylpropylideneaminyl, 2-methylpropylideneamino, butylideneamino, 1-methylbutylideneamino, 2-methylbutylideneamino, cyclopropylideneamino, cyclobutylideneamino, cyclopentylideneamino, cyclohexylideneamino and the like.

The term “carbocycle” refers to a group comprising a covalently closed ring, wherein each of the atoms forming the ring is a carbon atom. Carbocylic rings may be formed by three, four, five, six, seven, eight, nine, or more than nine carbon atoms. Carbocycles may be optionally substituted.

The term “heterocycle” refers to a group comprising a covalently closed ring wherein at least one atom forming the ring is a heteroatom. Heterocyclic rings may be formed by three, four, five, six, seven, eight, nine, or more than nine atoms. Any number of those atoms may be heteroatoms (i.e., a heterocyclic ring may comprise one, two, three, four, five, six, seven, eight, nine, or more than nine heteroatoms). In heterocyclic rings comprising two or more heteroatoms, those two or more heteroatoms may be the same or different from one another. Heterocycles may be optionally substituted. Binding to a heterocycle can be at a heteroatom or via a carbon atom. For example, binding for benzo-fused derivatives, may be via a carbon of the benzenoid ring. Examples of heterocycles include, but are not limited to the following

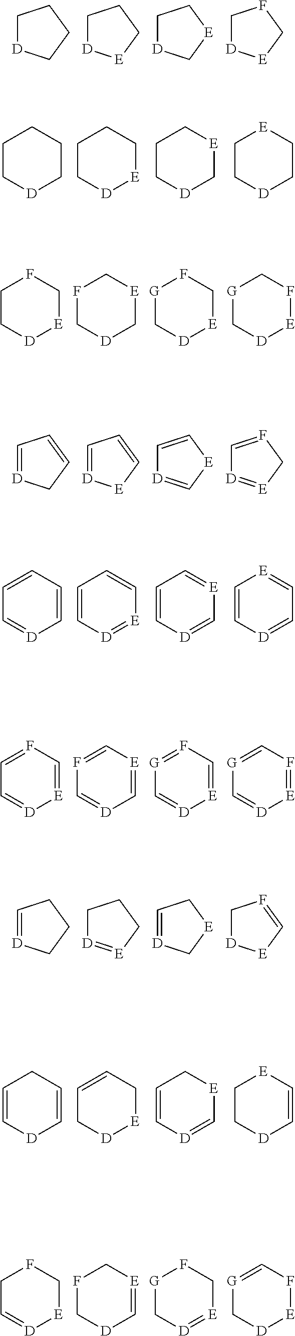

wherein D, E, F, and G independently represent a heteroatom. Each of D, E, F, and G may be the same or different from one another. Heterocycles may be aromatic heterocycles (i.e., heteroaryls) or non-aromatic heterocycles. In some embodiments, a non-aromatic heterocycle is a fully statured covalently closed ring (for example, piperidine, pyrrolidine, morpholine, piperazine, and the like).

The term “heteroatom” refers to an atom other than carbon or hydrogen. Heteroatoms are typically independently selected from oxygen, sulfur, nitrogen, and phosphorus, but are not limited to those atoms. In embodiments in which two or more heteroatoms are present, the two or more heteroatoms may all be the same as one another, or some or all of the two or more heteroatoms may each be different from the others.

The term “aromatic” refers to a group comprising a covalently closed ring having a delocalized π-electron system. Aromatic rings may be formed by five, six, seven, eight, nine, or more than nine atoms. Aromatics may be optionally substituted. Examples of aromatic groups include, but are not limited to phenyl, naphthalenyl, phenanthrenyl, anthracenyl, tetralinyl, fluorenyl, indenyl, and indanyl. The term aromatic includes, for example, benzenoid groups, connected via one of the ring-forming carbon atoms, and optionally carrying one or more substituents selected from an aryl, a heteroaryl, a cycloalkyl, a non-aromatic heterocycle, a halo, a hydroxy, an amino, a cyano, a nitro, an alkylamido, an acyl, a C₁₋₆alkoxy, a C₁₋₆alkyl, a C₁₋₆ hydroxyalkyl, a C₁₋₆ aminoalkyl, a C₁₋₆ alkylamino, an alkylsulfenyl, an alkylsulfinyl, an alkylsulfonyl, an sulfamoyl, or a trifluoromethyl. In certain embodiments, an aromatic group is substituted at one or more of the para, meta, and/or ortho positions. Examples of aromatic groups comprising substitutions include, but are not limited to, phenyl, 3-halophenyl, 4-halophenyl, 3-hydroxyphenyl, 4-hydroxyphenyl, 3-aminophenyl, 4-aminophenyl, 3-methylphenyl, 4-methylphenyl, 3-methoxyphenyl, 4-methoxyphenyl, 4-trifluoromethoxyphenyl, 3-cyanophenyl, 4-cyanophenyl, dimethylphenyl, naphthyl, hydroxynaphthyl, hydroxymethylphenyl, (trifluoromethyl)phenyl, alkoxyphenyl, 4-morpholin-4-ylphenyl, 4-pyrrolidin-1-ylphenyl, 4-pyrazolylphenyl, 4-triazolylphenyl, and 4-(2-oxopyrrolidin-1-yl)phenyl.

The term “aryl” refers to an aromatic group wherein each of the atoms forming the ring is a carbon atom. Aryl rings may be formed by five, six, seven, eight, nine, or more than nine carbon atoms. Aryl groups may be optionally substituted.

The term “heteroaryl” refers to an aromatic mono-, bi- or tricyclic ring system wherein at least one atom forming the aromatic ring system is a heteroatom. Heteroaryl rings may be formed by three, four, five, six, seven, eight, nine, or more than nine atoms. Heteroaryl groups may be optionally substituted. Examples of heteroaryl groups include, but are not limited to, aromatic C₃₋₈ heterocyclic groups comprising one oxygen or sulfur atom or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl groups are optionally substituted with one or more substituents, independently selected from halo, hydroxy, amino, cyano, nitro, alkylamido, acyl, C₁₋₆-alkoxy, C₁₋₆-alkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl. In some embodiments, the substituents are halo, hydroxy, cyano, O—C₁₋₆-alkyl, C₁₋₆-alkyl, hydroxy-C₁₋₆-alkyl, and amino-C₁₋₆-alkyl. Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline, and quinoxaline.

The term “non-aromatic ring” refers to a group comprising a covalently closed ring that does not have a delocalized π-electron system.

The term “non-aromatic heterocycle” refers to a group comprising a non-aromatic ring wherein one or more atoms forming the ring is a heteroatom. Non-aromatic heterocyclic rings may be formed by three, four, five, six, seven, eight, nine, or more than nine atoms. Non-aromatic heterocycles may be optionally substituted. In certain embodiments, non-aromatic heterocycles comprise one or more carbonyl or thiocarbonyl groups such as, for example, oxo- and thio-containing groups. Examples of non-aromatic heterocycles include, but are not limited to, lactams, lactones, cyclic imides, cyclic thioimides, cyclic carbamates, tetrahydrothiopyran, 4H-pyran, tetrahydropyran, piperidine, 1,3-dioxin, 1,3-dioxane, 1,4-dioxin, 1,4-dioxane, piperazine, 1,3-oxathiane, 1,4-oxathiin, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, morpholine, trioxane, hexahydro-1,3,5-triazine, tetrahydrothiophene, tetrahydrofuran, pyrroline, pyrrolidine, pyrrolidone, pyrrolidione, pyrazoline, pyrazolidine, imidazoline, imidazolidine, 1,3-dioxole, 1,3-dioxolane, 1,3-dithiole, 1,3-dithiolane, isoxazoline, isoxazolidine, oxazoline, oxazolidine, oxazolidinone, thiazoline, thiazolidine, and 1,3-oxathiolane.

The term “polycyclic heterocyclyl” used herein refers a bicyclic moiety or tricyclic moiety optionally containing one or more heteroatoms wherein at least one of the rings is an aryl or heteroaryl ring and at least one of the rings is non-aromatic. The bicyclic moiety contains two rings wherein the rings are fused. The bicyclic moiety can be appended at any position of the two rings. For example, bicyclic moiety may refer to a radical including but not limited to:

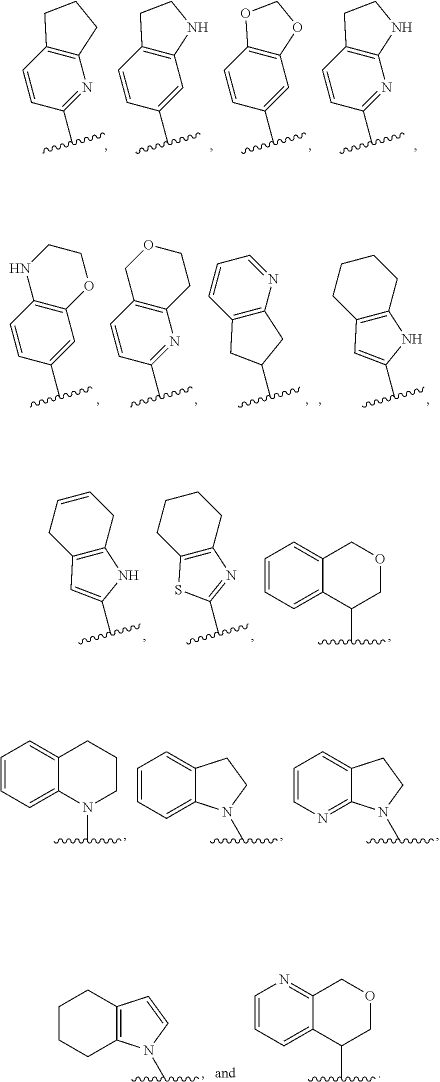

The tricyclic moiety contains a bicyclic moiety with an additional fused ring. The tricyclic moiety can be appended at any position of the three rings. For example, tricyclic moiety may refer to a radical including but not limited to:

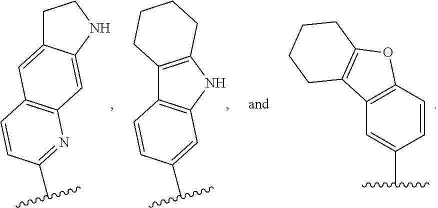

The term “arylalkyl” refers to a group comprising an aryl group bound to an alkyl group.

The term “carbocycloalkyl” refers to a group comprising a carbocyclic cycloalkyl ring. Carbocycloalkyl rings may be formed by three, four, five, six, seven, eight, nine, or more than nine carbon atoms. Carbocycloalkyl groups may be optionally substituted.

The term “ring” refers to any covalently closed structure. Rings include, for example, carbocycles (e.g., aryls and cycloalkyls), heterocycles (e.g., heteroaryls and non-aromatic heterocycles), aromatics (e.g., aryls and heteroaryls), and non-aromatics (e.g., cycloalkyls and non-aromatic heterocycles). Rings may be optionally substituted. Rings may form part of a ring system.

The term “ring system” refers to a either a single ring or two or more rings, wherein, if two or more rings are present, the two or more of the rings are fused. The term “fused” refers to structures in which two or more rings share one or more bonds.

The term “spacer” refers to an atom or group of atoms that separate two or more groups from one another by a desired number of atoms. For example, in certain embodiments, it may be desirable to separate two or more groups by one, two, three, four, five, six, or more than six atoms. In such embodiments, any atom or group of atoms may be used to separate those groups by the desired number of atoms. Spacers are optionally substituted. In certain embodiments, a spacer comprises saturated or unsaturated alkyls, heteroalkyls and/or haloalkyls. In certain embodiments, a spacer comprises atoms that are part of a ring.

Solely for the purposes of illustration, and without limiting the above definition, some examples of spacers are provided. Examples of 1 atom spacers include, but are not limited to, the following:

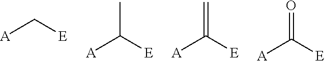

where A and E represent groups which are separated by the desired number of atoms. Examples of 2 atom spacers include, but are not limited to, the following

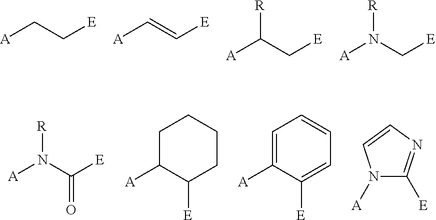

where A and E represent groups which are separated by the desired number of atoms.

Examples of 3 atom spacers include, but are not limited to, the following:

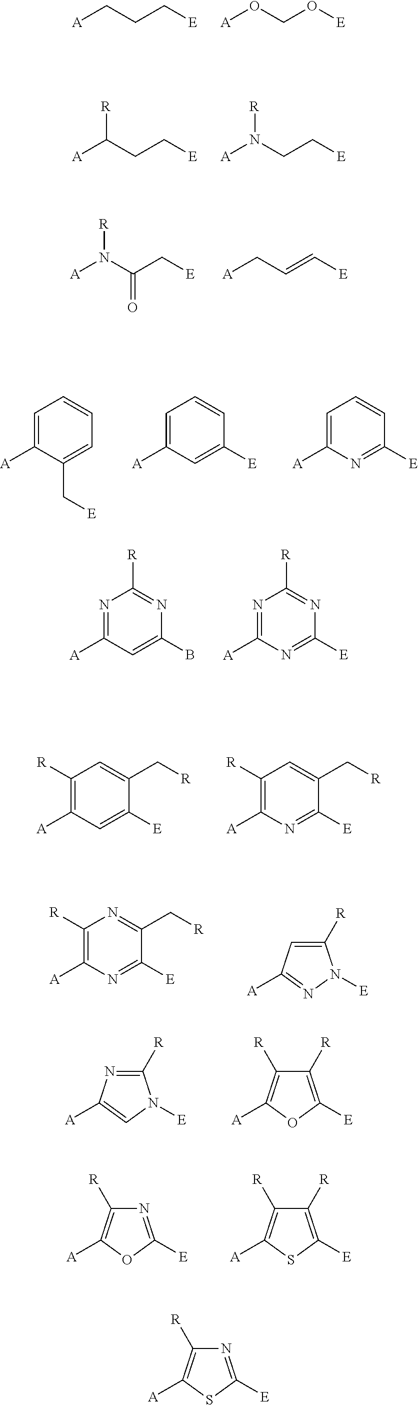

where A and E represent groups which are separated by the desired number of atoms. Examples of 4 atom spacers include, but are not limited to, the following:

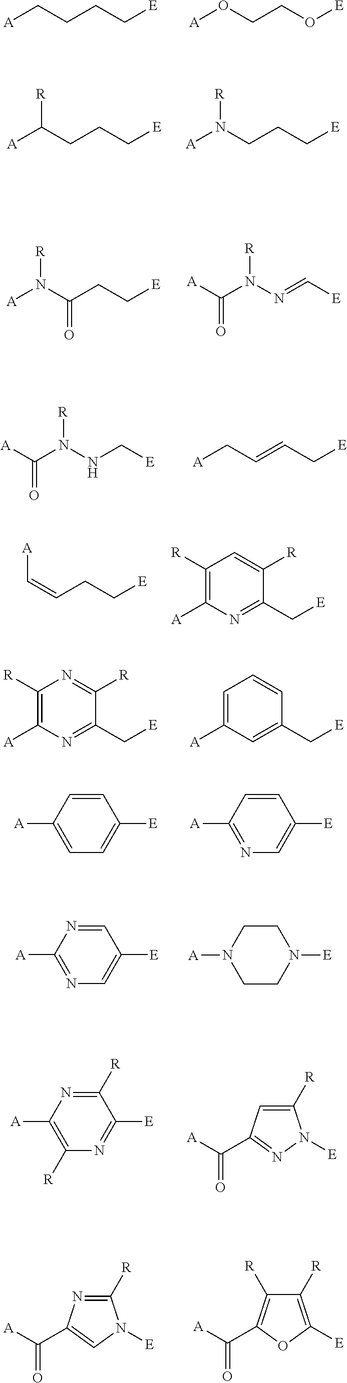

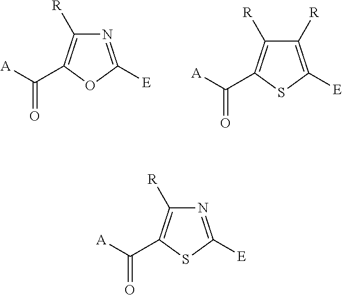

where A and E represent groups which are separated by the desired number of atoms. As is evident from the above examples, the atoms that create the desired separation may themselves be part of a group. That group may be, for example, an alkyl, heteroalkyl, haloalkyl, heterohaloalkyl, cycloalkyl, aryl, arylalkyl, heteroaryl, non-aromatic heterocycle, or substituted alkyl all of which are optionally substituted. Thus the term “1-5 atom spacer” refers to a spacer that separates two groups by 1, 2, 3, 4, or 5 atoms and does not indicate the total size of the group that constitutes the spacer.

As used herein, the term “linked to form a ring” refers to instances where two atoms that are bound either to a single atom or to atoms that are themselves ultimately bound, are each bound to a linking group, such that the resulting structure forms a ring. That resulting ring comprises the two atoms that are linked to form a ring, the atom (or atoms) that previously linked those atoms, and the linker. For example, if A and E below are “linked to form a ring”

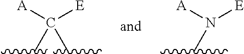

the resulting ring includes A, E, the C (carbon) or N (nitrogen) to which they are attached, and a linking group. Unless otherwise indicated, that linking group may be of any length and may be optionally substituted. Referring to the above example, resulting structures include, but are not limited to:

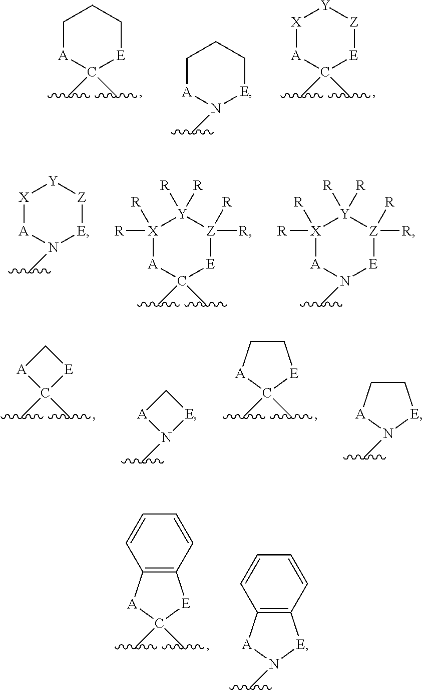

and the like.

In certain embodiments, the two substituents that together form a ring are not immediately bound to the same atom. For example, if A and E, below, are linked to form a ring:

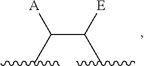

the resulting ring comprises A, E, the two atoms that already link A and E and a linking group. Examples of resulting structures include, but are not limited to:

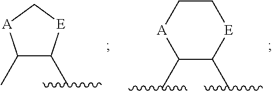

and the like.

In certain embodiments, the atoms that together form a ring are separated by three or more atoms. For example, if A and E, below, are linked to form a ring:

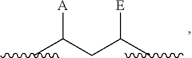

the resulting ring comprises A, E, the 3 atoms that already link A and E, and a linking group. Examples of resulting structures include, but are not limited to:

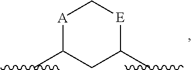

and the like.

As used herein, the term “together form a bond” refers to the instance in which two substituents to neighboring atoms are null the bond between the neighboring atoms becomes a double bond. For example, if A and E below “together form a bond”

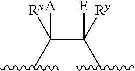

the resulting structure is:

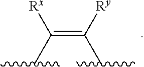

The term “null” refers to a group being absent from a structure. For example, in the structure

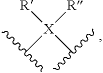

where in certain instances X is N (nitrogen), if X is N (nitrogen), one of R′ or R″ is null, meaning that only three groups are bound to the N (nitrogen).

The substituent “R” appearing by itself and without a number designation refers to a substituent selected from alkyl, cycloalkyl, aryl, heteroaryl (bonded through a ring carbon) and non-aromatic heterocycle (bonded through a ring carbon).

The term “O-carboxy” refers to the group consisting of formula RC(═O)O—.

The term “C-carboxy” refers to the group consisting of formula —C(═O)OR.

The term “acetyl” refers to the group consisting of formula —C(═O)CH₃.

The term “trihalomethanesulfonyl” refers to the group consisting of formula X₃CS(═O)₂— where X is a halogen.

The term “cyano” refers to the group consisting of formula —CN.

The term “isocyanato” refers to the group consisting of formula —NCO.

The term “thiocyanato” refers to the group consisting of formula —CNS.

The term “isothiocyanato” refers to the group consisting of formula —NCS.

The term “sulfonyl” refers to the group consisting of formula —S(═O)—R.

The term “S-sulfonamido” refers to the group consisting of formula —S(═O)₂NR.

The term “N-sulfonamido” refers to the group consisting of formula RS(═O)₂NH—.

The term “trihalomethanesulfonamido” refers to the group consisting of formula X₃CS(═O)₂NR—.

The term “O-carbamyl” refers to the group consisting of formula —OC(═O)—NR.

The term “N-carbamyl” refers to the group consisting of formula ROC(═O)NH—.

The term “O-thiocarbamyl” refers to the group consisting of formula —OC(═S)—NR.

The term “N-thiocarbamyl” refers to the group consisting of formula ROC(═S)NH—.

The term “C-amido” refers to the group consisting of formula —C(═O)—NR₂.

The term “N-amido” refers to the group consisting of formula RC(═O)NH—.

The term “oxo” refers to the group consisting of formula ═O.

The term “keto” and “carbonyl” used herein refers to C═O.

The term “thiocarbonyl” used herein refers to C═S.

The term “ester” refers to a chemical moiety with formula —(R)ₙ—C(═O)OR′, where R and R′ are independently selected from alkyl, cycloalkyl, aryl, heteroaryl (bonded through a ring carbon) and non-aromatic heterocycle (bonded through a ring carbon), where n is 0 or 1.

The term “amide” refers to a chemical moiety with formula —(R)ₙ—C(═O)NHR′ or —(R)ₙ—NHC(═O)R′, where R is selected from alkyl, cycloalkyl, aryl, heteroaryl (bonded through a ring carbon) and heteroalicyclic (bonded through a ring carbon), where n is 0 or 1 and R′ is selected from hydrogen, alkyl, cycloalkyl, aryl, heteroaryl (bonded through a ring carbon) and heteroalicyclic (bonded through a ring carbon), where n is 0 or 1. In certain embodiments, an amide may be an amino acid or a peptide.

The term “amino” refers to a chemical moiety with formula —NHR′R″, where R′ and R″ are each independently selected from hydrogen, alkyl, cycloalkyl, aryl, heteroaryl (bonded through a ring carbon) and heteroalicyclic (bonded through a ring carbon).

The terms “amine,” “hydroxy,” and “carboxyl” include such groups that have been esterified or amidified. Procedures and specific groups used to achieve esterification and amidification are known to those of skill in the art and can readily be found in reference sources such as Greene and Wuts, Protective Groups in Organic Synthesis, 3^{rd} Ed., John Wiley & Sons, New York, N.Y., 1999, which is incorporated herein in its entirety.

Unless otherwise indicated, the term “optionally substituted,” refers to a group in which none, one, or more than one of the hydrogen atoms has been replaced with one or more group(s) individually and independently selected from: alkyl, alkenyl, cycloalkenyl, alkynyl, heteroalkyl, haloalkyl, cycloalkyl, aryl, arylalkyl, alkenylO—, arylalkylO—, arylalkylNH—, alkenylO—, cycloalkylC(═O)—, arylC(═O)—, arylC(═O)NH—, arylNHC(═O)—, aryl(CH₂)₀₋₃O(CH₂)₁₋₃—, HO(CH₂)₁₋₃NH—, HO(CH₂)₁₋₃O—, HO(CH₂)₁₋₃—, HO(CH₂)₁₋₃O(CH₂)₁₋₃—, —C(═O)NHNH₂, heteroaryl, heterocycle, hydroxy, alkoxy, aryloxy, mercapto, alkylthio, arylthio, cyano, halo, carbonyl, oxo, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, and amino, including mono- and di-substituted amino groups, and the protected derivatives of amino groups. Such protective derivatives (and protecting groups that may form such protective derivatives) are known to those of skill in the art and may be found in references such as Greene and Wuts, above. When the group contains a nitrogen, or a sulfur, an oxo as a substituent also includes oxides, for example pyridine-N-oxide, thiopyran sulfoxide and thiopyran-S,S-dioxide. In embodiments in which two or more hydrogen atoms have been substituted, the substituent groups may together form a ring.

The term “stereoisomers” as used herein means isomers that possess identical constitution, but which differ in the arrangement of their atoms in space. Including, for example, all enantiomers, diastereomers, geometric isomers, and atropisomers.

Wherever a substituent as depicted as a di-radical (i.e., has two points of attachment to the rest of the molecule), it is to be understood that the substituent can be attached in any directional configuration unless otherwise indicated. Thus, for example, a substituent depicted as -AE- or

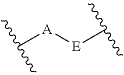

includes the substituent being oriented such that the A is attached at the leftmost attachment point of the molecule as well as attached at the rightmost attachment point of the molecule.

It is to be understood that certain radical naming conventions can include either a mono-radical or a di-radical, depending on the context. For example, where a substituent requires two points of attachment to the rest of the molecule, it is understood that the substituent is a di-radical. A substituent identified as alkyl, that requires two points of attachment, includes di-radicals such as —CH₂—, —CH₂CH₂—, —CH₂CH(CH₃)CH₂—, and the like; a substituent depicted as alkoxy that requires two points of attachment, includes di-radicals such as —OCH₂—, —OCH₂CH₂—, —OCH₂CH(CH₃)CH₂—, and the like: and a substituent depicted as arylC(═O)— that requires two points of attachment, includes di-radicals such as

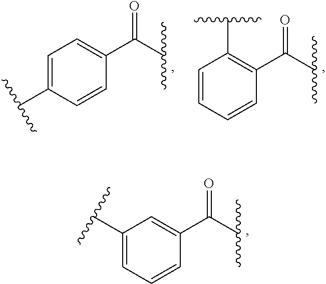

and the like.

Throughout the specification, groups and substituents thereof can be chosen by one skilled in the field to provide stable moieties and compounds.

The term “carrier” refers to a compound that facilitates the incorporation of another compound into cells or tissues. For example, dimethyl sulfoxide (DMSO) is a commonly used carrier for improving incorporation of certain organic compounds into cells or tissues.

The term “pharmaceutical agent” refers to a chemical compound or composition capable of inducing a desired therapeutic effect in a patient. In certain embodiments, a pharmaceutical agent comprises an active agent, which is the agent that induces the desired therapeutic effect. In certain embodiments, a pharmaceutical agent comprises a prodrug. In certain embodiments, a pharmaceutical agent comprises inactive ingredients such as carriers, excipients, and the like.

The term “therapeutically effective amount” refers to an amount of a pharmaceutical agent sufficient to achieve a desired therapeutic effect.

The term “prodrug” refers to an pharmaceutical agent that is converted from a less active form into a corresponding more active form in vivo.

The term “pharmaceutically acceptable” refers to a formulation of a compound that does not significantly abrogate the biological activity, a pharmacological activity and/or other properties of the compound when the formulated compound is administered to a patient. In certain embodiments, a pharmaceutically acceptable formulation does not cause significant irritation to a patient.

The term “co-administer” refers to administering more than one pharmaceutical agent to a patient. In certain embodiments, co-administered pharmaceutical agents are administered together in a single dosage unit. In certain embodiments, co-administered pharmaceutical agents are administered separately. In certain embodiments, co-administered pharmaceutical agents are administered at the same time. In certain embodiments, co-administered pharmaceutical agents are administered at different times.

The term “patient” includes human and animal subjects.

The term “substantially pure” means an object species (e.g., compound) is the predominant species present (i.e., on a molar basis it is more abundant than any other individual species in the composition). In certain embodiments, a substantially purified fraction is a composition wherein the object species comprises at least about 50 percent (on a molar basis) of all species present. In certain embodiments, a substantially pure composition will comprise more than about 80%, 85%, 90%, 95%, or 99% of all species present in the composition. In certain embodiments, the object species is purified to essential homogeneity (contaminant species cannot be detected in the composition by conventional detection methods) wherein the composition consists essentially of a single species.

The term “tissue-selective” refers to the ability of a compound to modulate a biological activity in one tissue to a greater or lesser degree than it modulates a biological activity in another tissue. The biological activities in the different tissues may be the same or they may be different. The biological activities in the different tissues may be mediated by the same type of target receptor. For example, in certain embodiments, a tissue-selective compound may modulate receptor mediated biological activity in one tissue and fail to modulate, or modulate to a lesser degree, receptor mediated biological activity in another tissue type.

The term “monitoring” refers to observing an effect or absence of any effect. In certain embodiments, one monitors cells after contacting those cells with a compound of the present embodiments. Examples of effects that may be monitored include, but are not limited to, changes in cell phenotype, cell proliferation, receptor activity, or the interaction between a receptor and a compound known to bind to the receptor.

The term “cell phenotype” refers to physical or biological characteristics. Examples of characteristics that constitute phenotype included, but are not limited to, cell size, cell proliferation, cell differentiation, cell survival, apoptosis (cell death), or the utilization of a metabolic nutrient (e.g., glucose uptake). Certain changes or the absence of changes in cell phenotype are readily monitored using techniques known in the art.

The term “cell proliferation” refers to the rate at which cells divide. In certain embodiments, cells are in situ in an organism. In certain embodiments, cell are grown in vitro in a vessel. The number of cells growing in a vessel can be quantified by a person skilled in the art (e.g., by counting cells in a defined area using a microscope or by using laboratory apparatus that measure the density of cells in an appropriate medium). One skilled in that art can calculate cell proliferation by determining the number of cells at two or more times.

The term “contacting” refers to bringing two or more materials into close enough proximity that they may interact. In certain embodiments, contacting can be accomplished in a vessel such as a test tube, a petri dish, or the like. In certain embodiments, contacting may be performed in the presence of additional materials. In certain embodiments, contacting may be performed in the presence of cells. In certain of such embodiments, one or more of the materials that are being contacted may be inside a cell. Cells may be alive or may dead. Cells may or may not be intact.

### Certain Compounds

Certain compounds that modulate one or more HGF activity and/or bind to HGF receptors play a role in health. In certain embodiments, compounds are useful for treating any of a variety of diseases or conditions.

Certain embodiments provide selective HGF modulators. Certain embodiments provide selective HGF receptor binding agents. Certain embodiments provide methods of making and methods of using selective HGF modulators and/or selective HGF receptor binding agents. In certain embodiments, selective HGF modulators are agonists, partial agonists, and/or antagonists for the HGF receptor.

The compounds disclosed herein can be used alone or in combination with other agents, for example, to modulate hematopoiesis, erythropoiesis, granulopoiesis, thrombopoiesis, and myelopoiesis. The instant compounds can also be used alone or in combination with other agents in treatment or prevention of a disease or condition caused by abnormal function of hematopoiesis, erythropoiesis, granulopoiesis, thrombopoiesis, and myelopoiesis. Some non-limiting examples of diseases include anemia, neutropenia, thrombocytopenia, cardiovascular disorders, immune/autoimmune disorders, cancers, infectious disorders or diseases, and neurologic disorders.

One of skill in the art will recognize that analogous synthesis schemes may be used to synthesize similar compounds. One of skill will recognize that compounds of the present embodiments may be synthesized using other synthesis schemes. In certain embodiments, a salt corresponding to any of the compounds provided herein is provided.

In certain embodiments, a salt corresponding to a selective HGF modulator is provided. In certain embodiments, a salt corresponding to a selective HGF receptor binding agent is provided. In certain embodiments, a salt is obtained by reacting a compound with an acid, such as hydrochloric acid, hydrobromic acid, sulfuric acid, nitric acid, phosphoric acid, methanesulfonic acid, ethanesulfonic acid, p-toluenesulfonic acid, salicylic acid, and the like. In certain embodiments, a salt is obtained by reacting a compound with a base to form a salt such as an ammonium salt, an alkali metal salt, such as a sodium or a potassium salt, an alkaline earth metal salt, such as a calcium or a magnesium salt, a salt of organic bases such as choline, dicyclohexylamine, N-methyl-D-glucamine, tris(hydroxymethyl)methylamine, 4-(2-hydroxyethyl)-morpholine, 1-(2-hydroxyethyl)-pyrrolidine, ethanolamine and salts with amino acids such as arginine, lysine, and the like. In certain embodiments, a salt is obtained by reacting a free acid form of a selective HGF modulator or selective HGF binding agent with multiple molar equivalents of a base, such as bis-sodium, bis-ethanolamine, and the like.

In certain embodiments, a salt corresponding to a compound of the present embodiments is selected from acetate, ammonium, benzenesulfonate, benzoate, bicarbonate, bisulfate, bitartrate, borate, bromide, calcium edetate, camsylate, carbonate, chloride, cholinate, clavulanate, citrate, dihydrochloride, diphosphate, edetate, edisylate, estolate, esylate, fumarate, gluceptate, gluconate, glutamate, glycollylarsanilate, hexylresorcinate, hydrabanine, hydrobromide, hydrochloride, hydroxynaphthoate, iodide, isethionate, lactate, lactobionate, laurate, magnesium, malate, maleate, mandelate, mucate, napsylate, nitrate, N-methylglucamine, oxalate, pamoate (embonate), palmitate, pantothenate, phosphate, polygalacturonate, potassium, salicylate, sodium, stearate, subaceatate, succinate, sulfate, tannate, tartrate, teoclate, tosylate, triethiodide, tromethamine, trimethylammonium, and valerate salts.

In certain embodiments, one or more carbon atoms of a compound of the present embodiments are replaced with silicon. See e.g., WO 03/037905A1; Tacke and Zilch, Endeavour, New Series, 10, 191-197 (1986); Bains and Tacke, Curr. Opin. Drug Discov Devel. Jul:6(4):526-43 (2003), all of which are incorporated herein by reference in their entirety. In certain embodiments, compounds comprising one or more silicon atoms possess certain desired properties, including, but not limited to, greater stability and/or longer half-life in a patient, when compared to the same compound in which none of the carbon atoms have been replaced with a silicon atom.

Some embodiments disclosed herein provide a compound of Formula I:

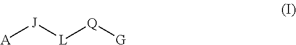

and pharmaceutically acceptable salts, esters, stereoisomers, tautomers or prodrugs thereof;

wherein:

A-J is

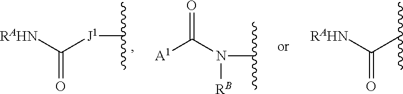

### and Q-G is

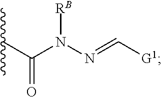

or A-J is

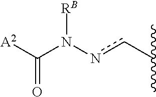

### and Q-G is

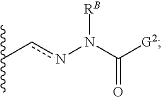

or A-J is

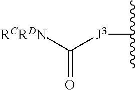

### and Q-G is

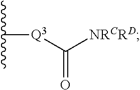

or A-J is

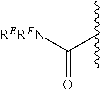

### and Q-G is

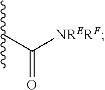

or A-J is

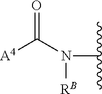

### and Q-G is

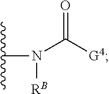

or A-J is

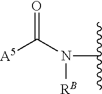

### and Q-G is

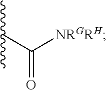

A¹ is selected from the group consisting of C₃-C₇ cycloalkenyl, C₃-C₇ cycloalkyl, C₁-C₆ alkyl, C₂-C₆ alkenyl, C₁-C₆ alkoxy, —(CH₂)ₘNR^{P}R^{L}, heterocycle, aryl, and heteroaryl, said C₃-C₇ cycloalkenyl, C₃-C₇ cycloalkyl, heterocycle, aryl, and heteroaryl, each optionally substituted with one or more substituents selected from the group consisting of R¹, R², and R³, said aryl and heteroaryl in the definition of A¹ are each further optionally fused with an optionally substituted nonaromatic heterocycle or an optionally substituted nonaromatic carbocycle;

J¹ is selected from the group consisting of —(CH₂)ᵣNR^{B}C(═O)(CH₂)ₘ— and —(CH₂)ᵣNR^{B}(CH₂)ₘ—, and —(CH₂)ᵣ—;

G¹ is selected from the group consisting of aryl and heteroaryl, each optionally substituted with one or more substituents selected from the group consisting of R⁴, R⁵, and R⁶, said aryl and heteroaryl in the definition of G¹ are each further optionally fused with an optionally substituted nonaromatic heterocycle or an optionally substituted nonaromatic carbocycle;

A² is selected from the group consisting of aryl and heteroaryl, each optionally substituted with one or more substituents selected from the group consisting of R¹, R², and R³, said aryl and heteroaryl in the definition of A² are each further optionally fused with an optionally substituted nonaromatic heterocycle or an optionally substituted nonaromatic carbocycle;

G² is selected from the group consisting of aryl and heteroaryl, each optionally substituted with one or more substituents selected from the group consisting of R⁴, R⁵, and R⁶, said aryl and heteroaryl in the definition of G² are each further optionally fused with an optionally substituted nonaromatic heterocycle or an optionally substituted nonaromatic carbocycle;

J³ is selected from the group consisting of an optionally substituted aryl, —(CH₂)ₘNR^{B}C(═O)(CH₂)ₘ—, —(CH₂)ᵣO(CH₂)ₘ—, —(CH₂)ᵣNR^{B}(CH₂)ₘ—, and —(CH═CH)ₘ—;

Q³ is selected from the group consisting of an optionally substituted aryl, —(CH₂)ᵣNR^{B}C(═O)(CH₂)ₘ—, —(CH₂)ᵣO(CH₂)ₘ—, —(CH₂)ᵣNR^{B}(CH₂)ₘ—, and —(CH═CH)ᵣ—;

A⁴ is selected from the group consisting of C₃-C₇ cycloalkenyl, C₃-C₇ cycloalkyl, C₁-C₆ alkyl, C₁-C₆ heteroalkyl, C₂-C₆ alkenyl, C₁-C₆ alkoxy, —(CH₂)ₘNR^{P}R^{L}, heterocycle, aryl, and heteroaryl, said C₃-C₇ cycloalkenyl, C₃-C₇ cycloalkyl, C₁-C₆ alkyl, C₁-C₆ heteroalkyl, C₂-C₆ alkenyl, heterocycle, aryl, and heteroaryl, each optionally substituted with one or more substituents selected from the group consisting of R¹, R², and R³, and said aryl and heteroaryl in the definition of A⁴ are each further optionally fused with an optionally substituted nonaromatic heterocycle or an optionally substituted nonaromatic carbocycle;

G⁴ is selected from the group consisting of C₃-C₇ cycloalkenyl, aryl, and heteroaryl, each optionally substituted with one or more substituents selected from the group consisting of R⁴, R⁵, and R⁶, said aryl and heteroaryl in the definition of G⁴ are each further optionally fused with an optionally substituted nonaromatic heterocycle or an optionally substituted nonaromatic carbocycle;

A⁵ is selected from the group consisting of aryl and heteroaryl, each optionally substituted with one or more substituents selected from the group consisting of R¹, R², and R³, said aryl and heteroaryl in the definition of A⁵ are each further optionally fused with an optionally substituted nonaromatic heterocycle or an optionally substituted nonaromatic carbocycle;

R^{A} is selected from the group consisting of —(CH₂)R^{L}, —(CH₂)ₚOR^{L}, —SO₂R^{L}, —C(═O)R^{L}, —C(═O)NR^{N}R^{O}, —(CH₂)NR^{N}R^{O}, an aryl and an heteroaryl, said aryl and heteroaryl in the definition of R^{A} are each optionally substituted with halogen, cyano, C₁-C₆ haloalkyl, C₁-C₆ alkyl, C₁-C₆ alkoxy, C₃-C₇ cycloalkyl, C₃-C₇ cycloalkenyl, —C(═O)NR^{N}R^{O}, —OC(═O)NR^{N}R^{O}, —NHC(═O)NR^{N}R^{O}, —O(CH₂)_{q}NR^{N}R^{O}, —NH(CH₂)_{q}NR^{N}R^{O}, —(CH₂)ₚNR^{N}R^{O}, an optionally substituted aryl and an optionally substituted heteroaryl, and said aryl and heteroaryl in the definition of R^{A} are each further optionally fused with an optionally substituted nonaromatic heterocycle or an optionally substituted nonaromatic carbocycle;

each R^{B} is separately selected from the group consisting of hydrogen, an optionally substituted C₁-C₆ alkyl, an optionally substituted C₂-C₆ alkenyl, and an optionally substituted C₃-C₇ cycloalkyl;

each —NR^{C}R^{D} is separately selected, wherein each R^{C} is independently selected from the group consisting of hydrogen and an optionally C₁-C₆ alkyl, and each R^{D} is independently selected from the group consisting of aryl and heteroaryl, said aryl and heteroaryl in the definition of R^{D} are each optionally substituted with halogen, cyano, C₁-C₆ alkyl, C₁-C₆ alkoxy, C₃-C₇ cycloalkyl, C₃-C₇ cycloalkenyl, —C(═O)NR^{N}R^{O}, —OC(═O)NR^{N}R^{O}, —NHC(═O)NR^{N}R^{O}, —O(CH₂)_{q}NR^{N}R^{O}, —NH(CH₂)_{q}NR^{N}R^{O}, —(CH₂)ₚNR^{N}R^{O}, an optionally substituted aryl and an optionally substituted heteroaryl, and said aryl and heteroaryl in the definition of R^{D} are each further optionally fused with an optionally substituted nonaromatic heterocycle or an optionally substituted nonaromatic carbocycle;

each —NR^{E}R^{F} is separately selected, wherein each R^{E} is independently selected from the group consisting of hydrogen and an optionally C₁-C₆ alkyl, and each RF is independently selected from the group consisting of aryl and heteroaryl, said aryl and heteroaryl in the definition of R^{F} are each optionally substituted with halogen, cyano, C₁-C₆ alkyl, C₁-C₆ alkoxy, C₃-C₇ cycloalkyl, C₃-C₇ cycloalkenyl, —C(═O)NR^{N}R^{O}, —OC(═O)NR^{N}R^{O}, —NHC(═O)NR^{N}R^{O}, —O(CH₂)_{q}NR^{N}R^{O}, —NH(CH₂)_{q}NR^{N}R^{O}, —(CH₂)ₚNR^{N}R^{O}, an optionally substituted aryl and an optionally substituted heteroaryl, and said aryl and heteroaryl in the definition of R^{F} are each further optionally fused with an optionally substituted nonaromatic heterocycle or an optionally substituted nonaromatic carbocycle;

R^{G} within the definition of —NR^{G}R^{H} is selected from the group consisting of C₁-C₆ alkyl, C₃-C₆ cycloalkyl, C₃-C₈ cycloalkenyl, C₁-C₆ heteroalkyl, C₁-C₆ heteroalkenyl, C₁-C₆ heteroalkynyl, heterocycle, aryl, and heteroaryl, each optionally substituted with one or more substituents selected from the group consisting of R⁴, R⁵, and R⁶, said aryl and heteroaryl in the definition of R^{G} are each further optionally fused with an optionally substituted nonaromatic heterocycle or an optionally substituted nonaromatic carbocycle, or R^{G} is —OR^{L} or —NR^{P}R^{L};

R^{H} within the definition of —NR^{G}R^{H} is selected from the group consisting of hydrogen, C₁-C₆ alkyl, C₂-C₆ alkenyl, C₃-C₇ cycloalkyl, and C₁-C₃ haloalkyl, or —NR^{G}R^{H} is an optionally substituted non-aromatic heterocycle linked through a ring nitrogen atom;

each R¹ is separately selected from the group consisting of halogen, cyano, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, optionally substituted C₃-C₇ cycloalkenyl, an optionally substituted C₁-C₆ haloalkyl, an optionally substituted C₁-C₆ heteroalkyl, an optionally substituted aryl, and an optionally substituted heteroaryl;

each R² is separately selected from the group consisting of halogen, —O(CH₂)ₘOR^{I}, —(CH₂)ₘOR^{I}, —NR^{J}R^{K}, —(CH₂)ₘSR^{I}, —C(═O)R^{L}, —(CH₂)ₘR^{L}, an optionally substituted C₁-C₆ alkyl, an optionally substituted C₁-C₆ alkoxy, an optionally substituted C₂-C₆ alkenyl, an optionally substituted C₁-C₆ haloalkyl, an optionally substituted C₁-C₆ heteroalkyl, and an optionally substituted C₃-C₇ cycloalkyl where said C₃-C₇ cycloalkyl is further optionally fused with aryl or heteroaryl;

each R³ is separately selected from the group consisting of halogen, —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, —(CH₂)ₘS(O)₀₋₂R^{M}, —(CH₂)ₘNHS(O)₀₋₂R^{M}, —(CH₂)ₘNO₂, —(CH₂)ₘCN, —(CH₂)ₘR^{P}, C₁-C₆ alkyl C₁-C₆ alkoxy, C₂-C₆ alkenyl, C₂-C₆ alkynyl, C₃-C₇ cycloalkyl, C₁-C₆ haloalkyl, heterocycle, aryl, and heteroaryl, said heterocycle, aryl and heteroaryl in the definition of R³ are each optionally substituted with halogen, cyano, C₁-C₆ alkyl, C₁-C₆ haloalkyl, C₁-C₆ alkoxy, or —NR^{J}R^{K};

each R⁴ is separately selected from the group consisting of halogen, cyano, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted C₁-C₆ haloalkyl, an optionally substituted C₁-C₆ heteroalkyl, an optionally substituted aryl, and an optionally substituted heteroaryl;

each R⁵ is separately selected from the group consisting of halogen, —O(CH₂)ₘOR^{I}, —(CH₂)ₘOR^{I}, —NR^{J}R^{K}, —(CH₂)ₘSR^{I}, —C(═O)R^{L}, —(CH₂)ₘR^{L}, an optionally substituted C₁-C₆ alkyl, an optionally substituted C₁-C₆ alkoxy, an optionally substituted C₂-C₆ alkenyl, an optionally substituted C₃-C₇ cycloalkyl, an optionally substituted C₁-C₆ haloalkyl, and an optionally substituted C₁-C₆ heteroalkyl;

each R⁶ is separately selected from the group consisting of halogen, —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, —(CH₂)ₘS(O)₀₋₂R^{M}, —(CH₂)ₘNHS(O)₀₋₂R^{M}, —(CH₂)ₘNO₂, —(CH₂)ₘCN, —(CH₂)ₘR^{P}, C₁-C₆ alkyl, C₁-C₆ alkoxy, C₂-C₆ alkenyl, C₂-C₆ alkynyl, C₃-C₇ cycloalkyl, C₁-C₆ haloalkyl, C₁-C₆ heteroalkyl, heterocycle, aryl, and heteroaryl, said heterocycle, aryl and heteroaryl in the definition of R⁶ are each optionally substituted with halogen, cyano, C₁-C₆ alkyl, C₁-C₆ haloalkyl, C₁-C₆ alkoxy, or —NR^{J}R^{K};

each R^{I} is separately selected from the group consisting of hydrogen, C₁-C₆ alkyl, C₂-C₄ alkenyl, C₂-C₄ alkynyl, C₃-C₇ cycloalkyl, C₁-C₆ haloalkyl, C₁-C₆ heteroalkyl, and C₁-C₆ heterohaloalkyl;

each —NR^{J}R^{K} is separately selected, wherein R^{J} and R^{K} are each independently selected from the group consisting of hydrogen, C₁-C₆ alkyl optionally substituted with up to 5 fluoro, —(CH₂)ₘOR^{JA}, —(CH₂)ₘNR^{JB}R^{JC}, —(CH₂)ₘR^{R}, C₃-C₇ cycloalkyl, heterocycle, aryl and heteroaryl, said C₃-C₇ cycloalkyl, heterocycle, aryl and heteroaryl in the definition of R^{J} and R^{K} are each independently optionally substituted with one or more substituents selected from the group consisting of halo, C₁-C₆ alkyl, C₁-C₆ haloalkyl, C₁-C₆ alkoxy, aryl and heteroaryl, said aryl and heteroaryl substituent off of R^{J} and R^{K} are each optionally substituted with one or more halo, C₁-C₆ alkyl, C₁-C₆ haloalkyl, C₁-C₆ alkoxy, or —(CH₂)ₘNR^{KA}R^{KB}; or —NR^{J}R^{K} is an optionally substituted non-aromatic heterocycle linked through a ring nitrogen atom; or —NR^{J}R^{K} is an optionally substituted C₁-C₆ alkylideneamino;

each R^{JA} is independently selected from the group consisting of hydrogen, C₁-C₆ alkyl, and C₁-C₆ haloalkyl;

each —NR^{JB}R^{JC} is separately selected, wherein R^{JB} and R^{JC} are each independently selected from the group consisting of hydrogen, C₁-C₆ alkyl, and C₁-C₆ haloalkyl;

each —NR^{KA}R^{KB} is separately selected, wherein R^{KA} and R^{KB} are each independently selected from the group consisting of hydrogen, C₁-C₆ alkyl, and C₁-C₆ haloalkyl;

each R^{M} is independently selected from the group consisting of hydrogen, an optionally substituted C₁-C₆ alkyl, an optionally substituted C₂-C₄ alkenyl, an optionally substituted C₂-C₄ alkynyl, an optionally substituted C₃-C₇ cycloalkyl, an optionally substituted C₃-C₇ cycloalkenyl, and —(CH₂)ₘR^{P};

each —NR^{N}R^{O} is separately selected, wherein R^{N} and R^{O} are each independently selected from the group consisting of hydrogen, —(CH₂)ₘNR^{NA}R^{NB}, aryl and heteroaryl, said aryl and heteroaryl in the definition of R^{N} and R^{O} are each independently optionally substituted with one or more substituents selected from the group consisting of —(CH₂)ₘNR^{OA}R^{OB}, halo, C₁-C₆ alkyl, C₁-C₆ haloalkyl, C₁-C₆ alkoxy, aryl and heteroaryl, said aryl and heteroaryl substituent off of R^{N} and R^{O} are each optionally substituted with one or more halo, C₁-C₆ alkyl, C₁-C₆ haloalkyl, C₁-C₆ alkoxy, or —NR^{NA}R^{NB}

each —NR^{NA}R^{NB} is separately selected, wherein R^{NA} and R^{NB} are each independently selected from the group consisting of hydrogen, C₁-C₆ alkyl, and C₁-C₆ haloalkyl;

each —NR^{OA}R^{O}B is separately selected, wherein R^{OA} and R^{OB} are each independently selected from the group consisting of hydrogen, C₁-C₆ alkyl, and C₁-C₆ haloalkyl;

R^{P} is selected from the group consisting of hydrogen and C₁-C₆ alkyl;

R^{L} is selected from the group consisting of C₃-C₇ cycloalkyl, optionally substituted C₁-C₆ alkyl, optionally substituted C₁-C₆ alkoxy, —(CH₂)ₘOR^{LA}, —(CH₂)ₘNR^{LB}R^{LC}, aryl and heteroaryl, said aryl and heteroaryl in the definition of R^{L} are each independently optionally substituted with one or more substituents selected from the group consisting of halo, C₁-C₆ alkyl, C₁-C₆ haloalkyl, C₁-C₆ alkoxy, —(CH₂)ₘNR^{LD}R^{LE} aryl and heteroaryl, said aryl and heteroaryl substituent off of R^{L} are each optionally substituted with one or more halo, C₁-C₆ alkyl, C₁-C₆ haloalkyl, C₁-C₆ alkoxy, or —(CH₂)ₘNR^{LF}R^{LG};

R^{LA} is selected from the group consisting of hydrogen, C₁-C₆ alkyl, and C₁-C₆ haloalkyl;

R^{LB} and R^{LC} are each independently selected from the group consisting of hydrogen, C₁-C₆ alkyl, C₁-C₆ haloalkyl, and C₁-C₆ heteroalkenyl; or —NR^{LB}R^{LC} is an optionally substituted non-aromatic heterocycle linked through a ring nitrogen atom;

each —NR^{LD}R^{LE} is separately selected, wherein R^{LD} and R^{LE} are each independently selected from the group consisting of hydrogen, aryl, heteroaryl, and optionally substituted C₁-C₆ alkyl, said aryl and heteroaryl in the definition of R^{LD} and R^{LE} are each optionally substituted with C₁-C₆ alkyl or C₁-C₆ alkoxy; or —NR^{LD}R^{LE} is an optionally substituted non-aromatic heterocycle linked through a ring nitrogen atom;

each —NR^{LF}R^{LG} is separately selected, wherein R^{LF} and R^{LG} are each independently selected from the group consisting of hydrogen, and C₁-C₆ alkyl; or —NR^{LF}R^{LG} is an optionally substituted non-aromatic heterocycle linked through a ring nitrogen atom;

L is selected from the group consisting of —O(CH₂)ₚO—, C₁-C₇ alkyl, C₁-C₇ heteroalkyl, C₃-C₇ cycloalkyl, C₃-C₇ cycloalkenyl, an optionally substituted heterocycle, an optionally substituted aryl, and an optionally substituted heteroaryl, or L is selected from the group consisting of L¹-L², L¹-O-L², L¹-S-L², L¹-NR⁹-L², L¹-L²-L³, L¹-L²-L³-L⁴, L¹-C(=E)-L², and L¹-CR⁷R-L²;

L¹ is selected from the group consisting of an optionally substituted aryl, an optionally substituted heteroaryl, and an optionally substituted heterocycle;

L² is selected from the group consisting of an optionally substituted aryl, an optionally substituted heteroaryl, and an optionally substituted heterocycle;

L³ is selected from the group consisting of an optionally substituted aryl, an optionally substituted heteroaryl, and an optionally substituted heterocycle;

L⁴ is an optionally substituted aryl;

E is O (oxygen), N—NHR^{Q} or N—OR^{Q} where R^{Q} in the definition of E is selected from the group consisting of hydrogen, an optionally substituted C₁-C₆ alkyl, an optionally substituted C₂-C₆ alkenyl, —(CH₂)ₘR^{R}, and —C(═O)(CH₂)ₘR^{R};

R^{R} is selected from the group consisting of C₁-C₆ alkyl, an optionally substituted aryl, and an optionally substituted heteroaryl;

R⁷ and R⁸ are each independently selected from the group consisting of hydrogen, C₁-C₆ alkyl, C₁-C₆ heteroalkyl, and —OH; or CR⁷R⁸ is a three- to eight-membered optionally substituted carbocycle, which optionally has one to three additional hetero atoms incorporated in the ring;

R⁹ is selected from the group consisting of hydrogen, C₁-C₆ alkyl, C₁-C₆ haloalkyl, C₁-C₆ heteroalkyl, C₃-C₇cycloalkylC(O)— and C₁-C₆ alkylC(O)—;

each m is independently 0, 1, 2, or 3;

each p is independently 0, 1, 2, 3, 4, 5, or 6;

each q is independently 1, 2, 3, 4, 5, or 6;

each r is independently 1, 2, 3, or 4; and

any bond represented by a dashed and solid line represents a bond selected from the group consisting of a single bond and a double bond.

Some embodiments disclosed herein provide a compound of Formula I, wherein R¹ can be selected from the group consisting of fluorine, chlorine, and methyl; R² can be selected from the group consisting of —(CH₂)ₘOR^{I}, —NR^{J}R^{K}, and —(CH₂)ₘSR^{I}; R³ can be selected from the group consisting of —(CH₂)ₘR^{P}, —(CH₂)ₘOR^{M}, and —NR^{N}R^{O}; R⁴ can be selected from the group consisting of fluorine, chlorine, and methyl; R⁵ can be selected from the group consisting of —(CH₂)ₘOR^{I}, —NR^{J}R^{K}, and —(CH₂)ₘSR^{I}; R⁶ can be selected from the group consisting of —(CH₂)ₘR^{P}, —(CH₂)ₘOR^{M}, and —NR^{N}R^{O}; R^{I} can be selected from the group consisting of hydrogen, C₁-C₆ alkyl, C₁-C₆ haloalkyl, and C₁-C₆ heteroalkyl; each —NR^{J}R^{K} can be separately selected, wherein R^{J} and R^{K} can each be independently selected from the group consisting of hydrogen, C₁-C₆ alkyl, C₁-C₆ haloalkyl, and C₁-C₆ heteroalkyl; or —NR^{J}R^{K} can be an optionally substituted non-aromatic heterocycle linked through a ring nitrogen atom; or —NR^{J}R^{K} can be an optionally substituted C₁-C₆ alkylideneamino; each R^{M} can be independently selected from the group consisting of hydrogen, an optionally substituted C₁-C₆ alkyl, an optionally substituted C₁-C₆ haloalkyl, an optionally substituted C₁-C₆ heteroalkyl, and —(CH₂)ₘR^{P}; R^{L} can be selected from the group consisting of hydrogen, C₁-C₃ alkyl, C₁-C₃ haloalkyl, and an optionally substituted aryl or an optionally substituted heteroaryl; and m can be 0, 1, or 2.

Some embodiments disclosed herein provide a compound of Formula I, wherein G¹ can be selected from the group consisting of aryl and heteroaryl, each substituted with one or more substituents selected from the group consisting of R⁴, R⁵, and R⁶, said aryl and heteroaryl in the definition of G¹ can each be further optionally fused with a nonaromatic heterocycle or nonaromatic carbocycle; A² can be selected from the group consisting of aryl and heteroaryl, each substituted with one or more substituents selected from the group consisting of R¹, R², and R³, said aryl and heteroaryl in the definition of A² can each be further optionally fused with a nonaromatic heterocycle or nonaromatic carbocycle; G² can be selected from the group consisting of aryl and heteroaryl, each substituted with one or more substituents selected from the group consisting of R⁴, R⁵, and R⁶, said aryl and heteroaryl in the definition of G² can each be further optionally fused with a nonaromatic heterocycle or nonaromatic carbocycle; A⁴ can be selected from the group consisting of C₃-C₇ cycloalkenyl, C₃-C₇ cycloalkyl, C₁-C₆ alkyl, C₁-C₆ heteroalkyl, C₂-C₆ alkenyl, C₁-C₆ alkoxy, —(CH₂)ₘNR^{P}R^{L}, heterocycle, aryl, and heteroaryl, said C₃-C₇ cycloalkenyl, C₃-C₇ cycloalkyl, C₁-C₆ alkyl, C₁-C₆ heteroalkyl, C₂-C₆ alkenyl, heterocycle, aryl, and heteroaryl, each substituted with one or more substituents selected from the group consisting of R¹, R², and R³, said aryl and heteroaryl in the definition of A⁴ can each be further optionally fused with a nonaromatic heterocycle or nonaromatic carbocycle; G⁴ can be selected from the group consisting of C₃-C₇ cycloalkenyl, aryl, and heteroaryl, each substituted with one or more substituents selected from the group consisting of R⁴, R⁵, and R⁶, said aryl and heteroaryl in the definition of G⁴ can each be further optionally fused with a nonaromatic heterocycle or nonaromatic carbocycle; A⁵ can be selected from the group consisting of aryl and heteroaryl, each substituted with one or more substituents selected from the group consisting of R¹, R², and R³, said aryl and heteroaryl in the definition of A⁵ can each be further optionally fused with a nonaromatic heterocycle or nonaromatic carbocycle; R¹ can be selected from the group consisting of fluorine, chlorine, and methyl; R² can be selected from the group consisting of —(CH₂)ₘOR^{I} and —NR^{J}R^{K}; R³ can be —R^{P}; R⁴ can be selected from the group consisting of fluorine, chlorine, and methyl; R⁵ can be selected from the group consisting of —(CH₂)ₘOR^{I} and —NR^{J}R^{K}; R⁶ can be —R^{P}; and R^{L} can be selected from the group consisting of C₁-C₃ alkyl, C₁-C₃ haloalkyl, an optionally substituted aryl and an optionally substituted heteroaryl.

Some embodiments disclosed herein provide a compound of Formula I, wherein L can be selected from the group consisting of an optionally substituted

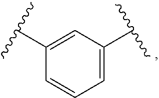 [SMILES: *c1cccc(*)c1]

an optionally substituted

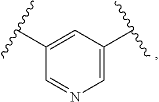 [SMILES: *c1cncc(*)c1]

an optionally substituted

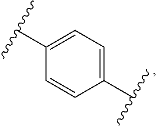 [SMILES: *c1ccc(*)cc1]

an optionally substituted

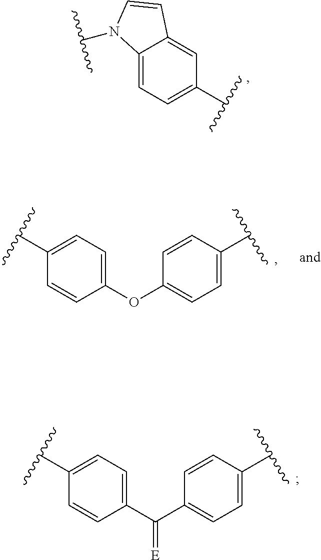

and E; and E can be O (oxygen), N—NHR^{Q} or N—OR^{Q} where R^{Q} in the definition of E can be selected from the group consisting of hydrogen, an optionally substituted C₁-C₆ alkyl, an optionally substituted C₂-C₄ alkenyl, —R^{R}, and —C(═O)R^{R}. In some embodiments, L can be selected from the group consisting of an optionally substituted

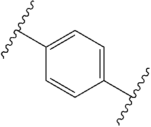 [SMILES: *c1ccc(*)cc1]

and an optionally substituted

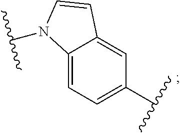 [SMILES: *c1ccc2c(ccn2*)c1]

each R¹ can be separately selected from the group consisting of an optionally substituted aryl and an optionally substituted heteroaryl; each R² can be separately selected from the group consisting of halogen, —(CH₂)ₘOR^{I}, and —NR^{B}R^{C}, where R^{I} in the definition of can be R² selected from the group consisting of hydrogen, and C₁-C₆ alkyl; each R³ can be fluoro; each —NR^{J}R^{K} can be separately selected, wherein R^{J} and R^{K} can each be independently selected from the group consisting of hydrogen and C₁-C₆ alkyl; or —NR^{J}R^{K} can be an optionally substituted non-aromatic heterocycle linked through a ring nitrogen atom; each R⁴ can be separately selected selected from the group consisting of chloro, fluoro, and an optionally substituted C₁-C₆ alkyl; each R⁵ can be separately selected from the group consisting of —OCH₂CH₂OR^{I}, —(CH₂)ₘOR^{I}, and —NR^{J}R^{K}, where R^{I} in the definition of can be R⁵ selected from the group consisting of hydrogen, and C₁-C₆ alkyl; and each R⁶ can be separately selected from the group consisting an optionally substituted aryl and an optionally substituted heteroaryl.

Some embodiments disclosed herein provide a compound of Formula I, wherein A¹, A², A⁴, and A⁵ can each be selected from the group consisting of phenyl, naphthyl, benzo[d][1,3]dioxolyl, indolyl, and benzo[d]imidazolyl, each substituted with one or more substituents selected from the group consisting of R¹ and R²; each R¹ can be separately selected from the group consisting of phenyl, pyrrolyl, and imidazolyl, each optionally substituted with a substituent selected from C₁-C₆ alkyl, C₁-C₆ alkoxy, C₁-C₆ alkylHN— and (C₁-C₆ alkyl)₂N—; each R² can be separately selected from the group consisting of bromo, chloro, fluoro, —(CH₂)ₘOR^{I}, —(CH₂)ₘR^{L}, and —NR^{J}R^{K}, where each R¹ in the definition of R² can be separately selected from the group consisting of hydrogen and C₁-C₆ alkyl; each —NR^{J}R^{K} can be separately selected, wherein R^{J} and R^{K} can each be independently selected from the group consisting of hydrogen, and C₁-C₆ alkyl optionally substituted with up to 3 fluoro; or —NR^{J}R^{K} can be a morpholinyl, piperazinyl, pyrrolidinyl, and piperidinyl, each optionally substituted with one or more oxo; each R^{L} can be separately selected from the group consisting aryl and heteroaryl, each optionally substituted with one or more substituents each separately selected from the group consisting of halogen, C₁-C₆ alkyl optionally substituted with up to 5 fluoro, and C₁-C₆ alkoxy optionally substituted with up to 5 fluoro; G¹, G², and G⁴ can each be selected from the group consisting of: phenyl, naphthyl, benzo[d][1,3]dioxolyl, indolyl, and benzo[d]imidazolyl, each substituted with one or more substituents selected from the group consisting of R⁴ and R⁵; each R⁴ can be separately selected from the group consisting of phenyl, pyrrolyl, and imidazolyl, each optionally substituted with a substituent selected from C₁-C₆ alkyl, C₁-C₆ alkoxy, C₁-C₆ alkylHN— and (C₁-C₆ alkyl)₂N—; and each R⁵ can be separately selected from the group consisting of bromo, chloro, fluoro, —(CH₂)ₘOR^{I}, and —NR^{J}R^{K}, where each R^{I} in the definition of R⁵ can be separately selected from the group consisting of hydrogen and C₁-C₆ alkyl.

Some embodiments disclosed herein provide a compound of Formula I, wherein A² can be selected from the group consisting of phenyl, naphthyl, and, indolyl, each substituted with one or more substituents selected from the group consisting of R¹ and R²; each R¹ can be separately selected from the group consisting of phenyl, pyrrolyl, and imidazolyl, each optionally substituted with a substituent selected from C₁-C₆ alkyl, C₁-C₆ alkoxy, C₁-C₆ alkylHN— and (C₁-C₆ alkyl)₂N—; each R² can be separately selected from the group consisting of bromo, chloro, fluoro, —(CH₂)ₘOR^{I}, —(CH₂)ₘR^{L}, and —NR^{J}R^{K}, where each R¹ in the definition of R² can be separately selected from the group consisting of hydrogen and C₁-C₆ alkyl; each —NR^{J}R^{K} can be separately selected, wherein R^{J} and R^{K} can each be independently selected from the group consisting of hydrogen, and C₁-C₆ alkyl optionally substituted with up to 3 fluoro; or —NR^{J}R^{K} can be a morpholinyl, piperazinyl, pyrrolidinyl, and piperidinyl, each optionally substituted with one or more oxo; each R^{L} can be separately selected from the group consisting aryl and heteroaryl, each optionally substituted with one or more substituents each separately selected from the group consisting of halogen, C₁-C₆ alkyl optionally substituted with up to 5 fluoro, and C₁-C₆ alkoxy optionally substituted with up to 5 fluoro; G² can be selected from the group consisting of: phenyl, naphthyl, and, indolyl, each substituted with one or more substituents selected from the group consisting of R⁴ and R⁵; each R⁴ can be separately selected from the group consisting of phenyl, pyrrolyl, and imidazolyl, each optionally substituted with a substituent selected from C₁-C₆ alkyl, C₁-C₆ alkoxy, C₁-C₆ alkylHN— and (C₁-C₆ alkyl)₂N—; and each R⁵ can be separately selected from the group consisting of bromo, chloro, fluoro, —(CH₂)ₘOR^{I}, and —NR^{J}R^{K}, where each R^{I} in the definition of R⁵ can be separately selected from the group consisting of hydrogen and C₁-C₆ alkyl.

Some embodiments disclosed herein provide a compound of Formula I, wherein A¹, A², A⁴, and A⁵ can each be selected from the group consisting of selected from the group consisting of a phenyl, naphthyl, benzo[d][1,3]dioxolyl, each substituted with one or more substituents selected from the group consisting of R¹, R², and R³; and G¹, G², and G⁴ can each be selected from the group consisting of aryl and heteroaryl, each substituted with one or more substituents selected from the group consisting of R⁴, R⁵, and R⁶ and each optionally fused with a nonaromatic heterocycle or carbocycle.

Some embodiments disclosed herein provide a compound of Formula I, wherein L can be selected from the group consisting of aryl, and heteroaryl, each substituted with one or more substituents each individually selected from the group consisting of alkyl, cycloalkyl, alkoxy, alkenylO—, arylC(═O)—, arylC(═O)NH—, arylNHC(═O)—, aryl(CH₂)₀₋₃O(CH₂)₀₋₃—, aryl(CH₂)₀₋₃NH(CH₂)₀₋₃—, HO(CH₂)₁₋₃NH—, HO(CH₂)₁₋₃O—, HO(CH₂)₁₋₃—, HO(CH₂)₁₋₃O(CH₂)₁₋₃—, and amino.

Some embodiments disclosed herein provide a compound of Formula I, wherein L can be L¹-L², or L¹-O-L², or L¹-NR⁹-L², or L¹-L²-L³, or L¹-L²-L³-L⁴, or L¹-C(=E)-L².

Some embodiments disclosed herein provide a compound of Formula I, wherein L¹ can be selected from the group consisting of aryl and heteroaryl, each substituted with one or more substituents each individually selected from the group consisting of alkyl, cycloalkyl, alkoxy, alkenylO—, arylC(═O)—, arylC(═O)NH—, arylNHC(═O)—, aryl(CH₂)₀₋₃O(CH₂)₀₋₃—, aryl(CH₂)₀₋₃NH(CH₂)₀₋₃—, HO(CH₂)₁₋₃NH—, HO(CH₂)₁₋₃O—, HO(CH₂)₁₋₃—, HO(CH₂)₁₋₃O(CH₂)₁₋₃—, and amino.

Some embodiments disclosed herein provide a compound of Formula I, wherein L² can be selected from the group consisting of aryl, heteroaryl, and heterocyclyl, each substituted with one or more substituents each individually selected from the group consisting of alkyl, cycloalkyl, alkoxy, alkenylO—, arylC(═O)—, arylC(═O)NH—, arylNHC(═O)—, aryl(CH₂)₀₋₃O(CH₂)₀₋₃—, aryl(CH₂)₀₋₃NH(CH₂)₀₋₃—, HO(CH₂)₁₋₃NH—, HO(CH₂)₁₋₃O—, HO(CH₂)₁₋₃—, HO(CH₂)₁₋₃O(CH₂)₁₋₃—, and amino.

Some embodiments disclosed herein provide a compound of Formula I, wherein L³ can be selected from the group consisting of aryl, heteroaryl, and heterocyclyl, each substituted with one or more substituents each individually selected from the group consisting of alkyl, cycloalkyl, alkoxy, alkenylO—, arylC(═O)—, arylC(═O)NH—, arylNHC(═O)—, aryl(CH₂)₀₋₃O(CH₂)₀₋₃—, aryl(CH₂)₀₋₃NH(CH₂)₀₋₃—, HO(CH₂)₁₋₃NH—, HO(CH₂)₁₋₃O—, HO(CH₂)₁₋₃—, HO(CH₂)₁₋₃O(CH₂)₁₋₃—, and amino.

Some embodiments disclosed herein provide a compound of Formula I, wherein L⁴ can be aryl substituted with one or more substituents each individually selected from the group consisting of alkyl, cycloalkyl, alkoxy, alkenylO—, arylC(═O)—, arylC(═O)NH—, arylNHC(═O)—, aryl(CH₂)₀₋₃O(CH₂)₀₋₃—, aryl(CH₂)₀₋₃NH(CH₂)₀₋₃—, HO(CH₂)₁₋₃NH—, HO(CH₂)₁₋₃O—, HO(CH₂)₁₋₃—, HO(CH₂)₁₋₃O(CH₂)₁₋₃—, and amino.

Some embodiments disclosed herein provide a compound of Formula I, having the proviso that a compound for Formula I is not selected from the group consisting of:

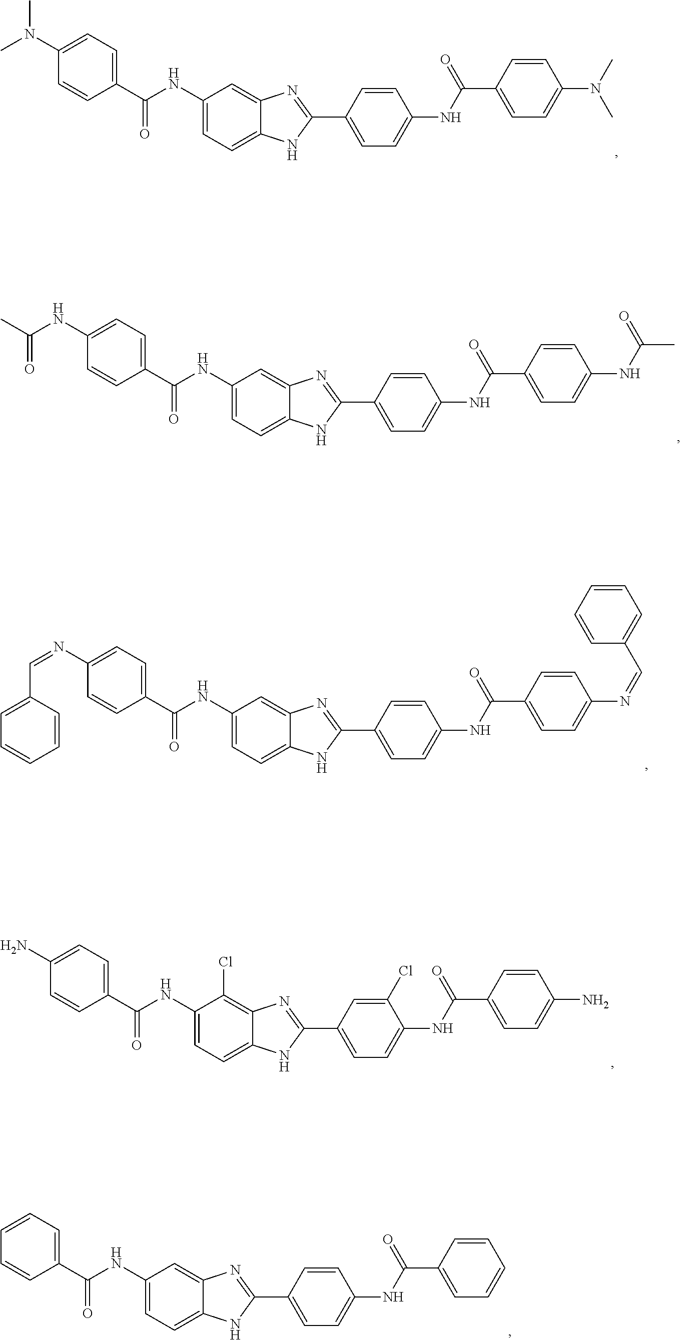

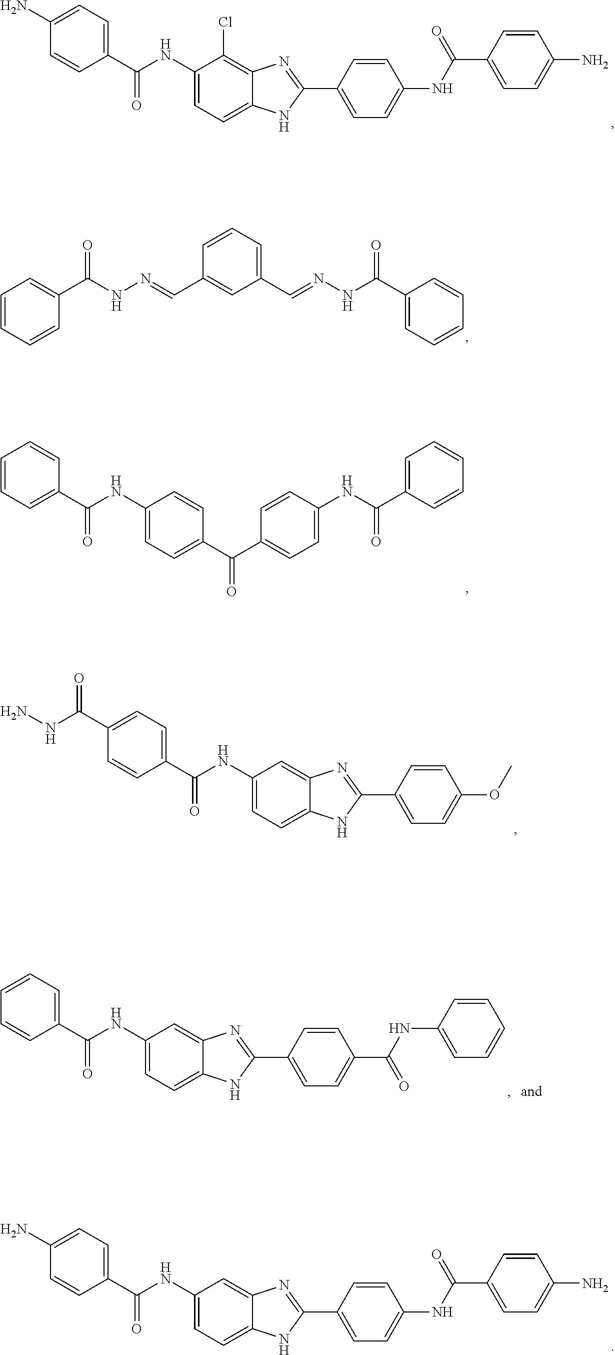 [SMILES: Nc1ccc(C(=O)Nc2ccc(-c3nc4c(Cl)c(NC(=O)c5ccc(N)cc5)ccc4[nH]3)cc2)cc1 ; O=C(N/N=C/c1cccc(/C=N/NC(=O)c2ccccc2)c1)c1ccccc1 ; O=C(Nc1ccc(C(=O)c2ccc(NC(=O)c3ccccc3)cc2)cc1)c1ccccc1 ; COc1ccc(-c2nc3cc(NC(=O)c4ccc(C(=O)NN)cc4)ccc3[nH]2)cc1 ; O=C(Nc1ccccc1)c1ccc(-c2nc3cc(NC(=O)c4ccccc4)ccc3[nH]2)cc1 ; Nc1ccc(C(=O)Nc2ccc(-c3nc4cc(NC(=O)c5ccc(N)cc5)ccc4[nH]3)cc2)cc1]

Some embodiments disclosed herein provide a compound of Formula I having the formula Ia:

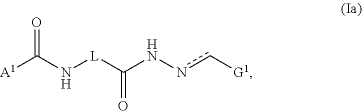

and pharmaceutically acceptable salts thereof, having the formula Ib:

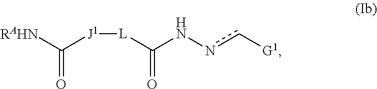

and pharmaceutically acceptable salts thereof, or having the formula Ic:

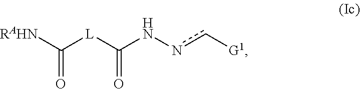

and pharmaceutically acceptable salts thereof.

Some embodiments disclosed herein provide a compound of Formula Ia, Ib, Ic, or Id, wherein L can be selected from the group consisting of an optionally substituted aryl, and an optionally substituted heteroaryl.

Some embodiments disclosed herein provide a compound of Formula I having the formula Id:

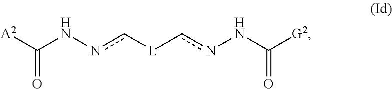

and pharmaceutically acceptable salts thereof.

Some embodiments disclosed herein provide a compound of Formula I having the structure of Formula Ie:

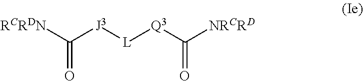

and pharmaceutically acceptable salts thereof.

Some embodiments disclosed herein provide a compound of Formula Ie, wherein L can be selected from the group consisting of —O(CH₂)ₚO—, an optionally substituted aryl, and an optionally substituted heteroaryl, or L is L¹-L²; J³ can be selected from the group consisting of an optionally substituted aryl, —(CH₂)ₘNR^{B}C(═O)—, —(CH₂)ᵣO—, and —(CH═CH)ₘ—; Q³ can be selected from the group consisting of an optionally substituted aryl, —(CH₂)ᵣNR^{B}C(═O)—, —(CH₂)ᵣO—, and —(CH═CH)ᵣ—; L¹ can be selected from the group consisting of an optionally substituted aryl, and an optionally substituted heteroaryl; and L² can be selected from the group consisting of an optionally substituted aryl, and an optionally substituted heteroaryl. In some embodiments, L can be —O(CH₂)ₚO—.

Some embodiments disclosed herein provide a compound of Formula I having the structure of Formula If:

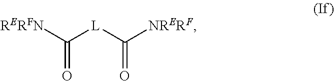

and pharmaceutically acceptable salts thereof.

Some embodiments disclosed herein provide a compound of Formula If, wherein L can be selected from the group consisting of C₄-C₆ cycloalkyl, an optionally substituted aryl, and an optionally substituted heteroaryl, or L is L¹-L²; L¹ can be selected from the group consisting of an optionally substituted aryl, and an optionally substituted heteroaryl; and L² can be selected from the group consisting of an optionally substituted aryl, and an optionally substituted heteroaryl.

Some embodiments disclosed herein provide a compound of Formula I having the structure of Formula Ig:

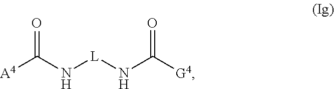

and pharmaceutically acceptable salts thereof.

Some embodiments disclosed herein provide a compound of Formula Ig, wherein L can be selected from the group consisting of an optionally substituted aryl, and an optionally substituted heteroaryl, or L can be selected from the group consisting of L¹-L², L¹-O-L², L¹-S-L², L¹-NR⁹-L², L¹-L²-L³, L¹-L²-L³-L⁴, L¹-C(=E)-L², and L¹-CR⁷R⁸-L²; L¹ can be selected from the group consisting of an optionally substituted aryl, and an optionally substituted heteroaryl; L² can be selected from the group consisting of an optionally substituted aryl, an optionally substituted heteroaryl, and an optionally substituted heterocycle; L³ can be selected from the group consisting of an optionally substituted aryl, and an optionally substituted heteroaryl; and L⁴ is an optionally substituted aryl.

Some embodiments disclosed herein provide a compound of Formula I having the structure of Formula Ih:

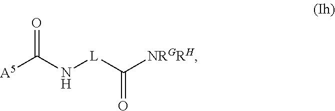

and pharmaceutically acceptable salts thereof.

Some embodiments disclosed herein provide a compound of Formula Ih, wherein L can be selected from the group consisting of an optionally substituted aryl or L can be selected from the group consisting of L¹-L², and L¹-L²-L; L¹ can be an optionally substituted heteroaryl; L² can be selected from the group consisting of an optionally substituted aryl, an optionally substituted heteroaryl, and an optionally substituted heterocycle; and L³ can be an optionally substituted heterocycle.

Some embodiments disclosed herein provide a compound of Formula II:

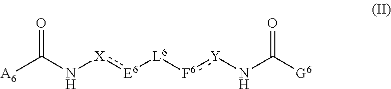

and pharmaceutically acceptable salts, esters, stereoisomers, tautomers or prodrugs thereof;

wherein:

A⁶ is selected from the group consisting of aryl and heteroaryl, each optionally substituted with one or more substituents selected from the group consisting of R¹¹, R¹², and R¹³, said aryl and heteroaryl in the definition of A⁶ are each further optionally fused with an optionally substituted nonaromatic heterocycle or an optionally substituted nonaromatic carbocycle;

G⁶ is selected from the group consisting of aryl and heteroaryl, each optionally substituted with one or more substituents selected from the group consisting of R¹⁴, R¹⁵, and R¹⁶, said aryl and heteroaryl in the definition of G⁶ are each further optionally fused with an optionally substituted nonaromatic heterocycle or an optionally substituted nonaromatic carbocycle;

L⁶ is an optionally substituted aryl, or an optionally substituted heteroaryl; where the aryl and heteroaryl in the definition of L⁶ are optionally fused with a nonaromatic heterocycle or a nonaromatic carbocycle; or L⁶ is selected from the group consisting of an optionally substituted

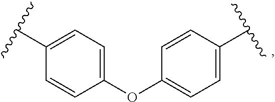 [SMILES: *c1ccc(Oc2ccc(*)cc2)cc1]

and an optionally substituted

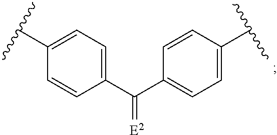

E² is O (oxygen) or N—OR^{D} where R^{D} in the definition of E² is selected from the group consisting of hydrogen and an optionally substituted C₁-C₆ alkyl;

each R¹¹ is separately selected from the group consisting of halogen, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₁-C₆ haloalkyl, and an optionally substituted C₁-C₆ heteroalkyl, an optionally substituted aryl, and an optionally substituted heteroaryl;

each R¹² is separately selected from the group consisting of —O(CH₂)ₘOR^{A}, —(CH₂)ₘOR^{A}, —NR^{B}R^{C}, and —(CH₂)ₘSR^{A};

each R¹³ is separately selected from the group consisting of —(CH₂)ₘOR^{D}, —NR^{E}R^{F}, —S(O)₀₋₂R^{D}, —(CH₂)ₘNO₂, —(CH₂)ₘCN, and —(CH₂)ₘR^{G};

each R¹⁴ is separately selected from the group consisting of halogen, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl, an optionally substituted aryl, and an optionally substituted heteroaryl;

each R¹⁵ is separately selected from the group consisting of —O(CH₂)ₘOR^{A}, —(CH₂)ₘOR^{A}, —NR^{B}R^{C}, and —(CH₂)ₘSR^{A};

each R¹⁶ is separately selected from the group consisting of —(CH₂)ₘOR^{D}, —NR^{E}R^{D}, —(CH₂)ₘS(O)₀₋₂R^{D}, —(CH₂)ₘNO₂, —(CH₂)ₘCN, and —(CH₂)ₘR^{G};

E⁶ is CR¹⁷ when the dashed line between E⁶ and X represents a double bond; or E⁶ is CR¹⁷R¹⁷ when the dashed line between E⁶ and X represents a single bond;

F⁶ is CR¹⁸ when the dashed line between F⁶ and Y represents a double bond; or F⁶ is CR¹⁸R¹⁸ when the dashed line between F⁶ and Y represents a single bond;

each R¹⁷ is independently selected from the group consisting of hydrogen, halogen, an optionally substituted C₁-C₄ alkoxy, an optionally substituted C₃-C₇ cycloalkyl, and an optionally substituted C₁-C₄ alkyl;

each R¹⁸ is independently selected from the group consisting of hydrogen, halogen, an optionally substituted C₁-C₄ alkoxy, an optionally substituted C₃-C₇ cycloalkyl, and an optionally substituted C₁-C₄ alkyl;

R^{A} is selected from the group consisting of hydrogen, C₁-C₆ alkyl, C₂-C₄ alkenyl, C₂-C₄ alkynyl, C₃-C₇ cycloalkyl, C₁-C₆ haloalkyl, C₁-C₆ heteroalkyl, and C₁-C₆ heterohaloalkyl;

each —NR^{B}R^{C} is separately selected, wherein R^{B} and R^{C} are each independently selected from the group consisting of hydrogen, —SO₂R^{H}, —C(═O)R^{H}, —C(═O)NR^{E}R^{F}, heterocycle, C₁-C₆ alkyl, C₂-C₄ alkenyl, an optionally substituted C₂-C₄ alkynyl, C₃-C₇ cycloalkyl, C₁-C₆ haloalkyl, C₁-C₆ heteroalkyl, and C₁-C₆ heterohaloalkyl, where the C₁-C₆ alkyl in the definition of R^{B} and R^{C} is optionally substituted with an optionally substituted aryl or an optionally substituted heteroaryl and where the C₃-C₇ cycloalkyl and the heterocycle in the definition of R^{B} and R^{C} are optionally fused with an aryl or heteroaryl; or —NR^{B}R^{C} is an optionally substituted non-aromatic heterocycle linked through a ring nitrogen atom; or —NR^{B}R^{C} is an optionally substituted C₁-C₆ alkylideneamino;

each R^{D} is independently selected from the group consisting of hydrogen, an optionally substituted C₁-C₆ alkyl, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl, and —(CH₂)ₘR^{G};

each —NR^{E}R^{F} is separately selected, wherein R^{E} and R^{F} are each independently selected from the group consisting of hydrogen, an optionally substituted C₁-C₆ alkyl, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl, and —(CH₂)ₘR^{G}; or —NR^{E}R^{F} is an optionally substituted C₁-C₆ alkylideneamino; or —NR^{E}R^{F} is an optionally substituted non-aromatic heterocycle linked through a ring nitrogen atom; R^{G} is selected from an optionally substituted aryl and an optionally substituted heteroaryl;

R^{H} is selected from the group consisting of hydrogen, C₁-C₃ alkyl, an optionally substituted C₁-C₃ alkoxy, an optionally substituted C₂-C₄ alkenyl, an optionally substituted C₂-C₄ alkynyl, an optionally substituted C₃-C₇ cycloalkyl, C₂-C₄ alkenyl, C₂-C₄ alkynyl, C₃-C₇ cycloalkyl, C₁-C₃ haloalkyl, and an optionally substituted aryl or heteroaryl;

X and Y are independently selected from N (nitrogen), NH, CR¹⁹, and CR¹⁹R²⁰

each R¹⁹ and R²⁰ are independently selected from the group consisting of hydrogen and an optionally substituted C₁-C₄ alkyl;

each m is independently 0, 1, or 2; and

any bond represented by a dashed and solid line represents a bond selected from the group consisting of a single bond and a double bond.

Some embodiments disclosed herein provide a compound of Formula II, wherein A⁶ can be selected from the group consisting of aryl and heteroaryl, each optionally substituted with one or more substituents selected from the group consisting of R¹¹, R¹², and R¹³, said aryl and heteroaryl in the definition of A⁶ can each be further optionally fused with a nonaromatic heterocycle or carbocycle; L⁶ can be selected from the group consisting of

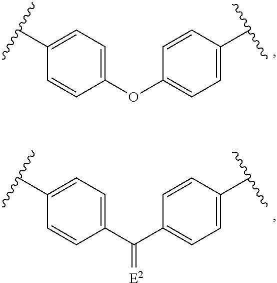

an optionally substituted

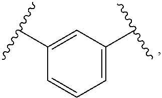 [SMILES: *c1cccc(*)c1]

an optionally substituted

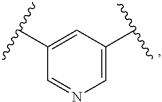 [SMILES: *c1cncc(*)c1]

an optionally substituted

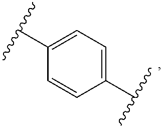 [SMILES: *c1ccc(*)cc1]

and an optionally substituted

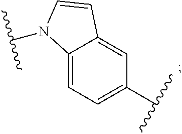 [SMILES: *c1ccc2c(ccn2*)c1]

E² can be O (oxygen) or N—OR^{D} where R^{D} in the definition of E² can be selected from the group consisting of hydrogen and an optionally substituted C₁-C₆ alkyl; G⁶ can be selected from the group consisting of aryl and heteroaryl, each optionally substituted with one or more substituents selected from the group consisting of R¹⁴, R¹⁵, and R¹⁶, said aryl and heteroaryl in the definition of G⁶ can each be further optionally fused with a nonaromatic heterocycle or carbocycle; each R¹¹ can be separately selected from the group consisting of fluoro, an optionally substituted aryl and an optionally substituted heteroaryl; each R¹² can be separately selected from the group consisting of —O(CH₂)ₘOR^{A}, —(CH₂)ₘOR^{A}, and —NR^{B}R^{C}, where R^{A} in the definition of can be R¹² selected from the group consisting of hydrogen, and C₁-C₆ alkyl; each R¹³ can be separately selected from the group consisting of —OR^{D}, —NR^{E}R^{F}, —S(O)₂R^{D}, —CN, and —R^{G}; each —NR^{B}R^{C} can be separately selected, wherein R^{B} and R^{C} can each be independently selected from the group consisting of hydrogen, C₃-C₇ cycloalkyl, C₁-C₆ alkyl, and C₁-C₆ haloalkyl, where the C₁-C₆ alkyl in the definition of R^{B} and R^{C} can be optionally substituted with an optionally substituted aryl or an optionally substituted heteroaryl, and where the C₃-C₇ cycloalkyl in the definition of R^{B} and R^{C} can be optionally fused with an optionally substituted aryl; or —NR^{B}R^{C} can be an optionally substituted non-aromatic heterocycle linked through a ring nitrogen atom; RH can be selected from the group consisting of hydrogen, C₁-C₃ alkyl, an optionally substituted aryl and an optionally substituted heteroaryl; each R¹⁴ can be separately selected selected from the group consisting of chloro, fluoro, an optionally substituted C₁-C₆ alkyl, an optionally substituted aryl, and an optionally substituted heteroaryl; each R¹⁵ can be separately selected from the group consisting of —O(CH₂)ₘOR^{A}, —(CH₂)ₘOR^{A}, and —NR^{B}R^{C}, where R^{A} in the definition of R¹⁵ can be selected from the group consisting of hydrogen, and C₁-C₆ alkyl; and each R¹⁶ can be separately selected from the group consisting of —OR^{D}, —NR^{E}R^{F}, —S(O)₂R^{D}, —CN, and —R^{G}. In some embodiments, A⁶ can be selected from the group consisting of aryl and heteroaryl, each substituted with one or more substituents selected from the group consisting of R¹¹, R¹², and R¹³, said aryl in the definition of A⁶ can each be further optionally fused with a nonaromatic heterocycle or nonaromatic carbocycle; G⁶ can be selected from the group consisting of aryl and heteroaryl, each substituted with one or more substituents selected from the group consisting of R¹⁴, R¹⁵, and R¹⁶, said aryl and heteroaryl in the definition of G⁶ can each be further optionally fused with a nonaromatic heterocycle or carbocycle; each R¹¹ can be separately selected from the group consisting of an optionally substituted aryl and an optionally substituted heteroaryl; each R¹² can be separately selected from the group consisting of —(CH₂)ₘOR^{A}, and —NR^{B}R^{C}, where R^{A} in the definition of R¹² can be selected from the group consisting of hydrogen, and C₁-C₆ alkyl; each R¹³ can be fluoro; each —NR^{B}R^{C} can be separately selected, wherein R^{B} and R^{C} can each be independently selected from the group consisting of hydrogen, —C(═O)R^{H}, and C₁-C₆ alkyl; or —NR^{B}R^{C} can be an optionally substituted non-aromatic heterocycle linked through a ring nitrogen atom; each R¹⁴ can be separately selected selected from the group consisting of chloro, fluoro, an optionally substituted C₁-C₆ alkyl, an optionally substituted aryl and an optionally substituted heteroaryl; each R¹⁵ can be separately selected from the group consisting of —OCH₂CH₂OR^{A}, —(CH₂)ₘOR^{A}, and —NR^{B}R^{C}, where R^{A} in the definition of can be R¹⁵ selected from the group consisting of hydrogen, and C₁-C₆ alkyl; and each R¹⁶ can be fluoro.

Some embodiments disclosed herein provide a compound of Formula II, wherein

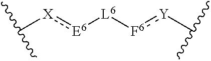

is selected from the group consisting of:

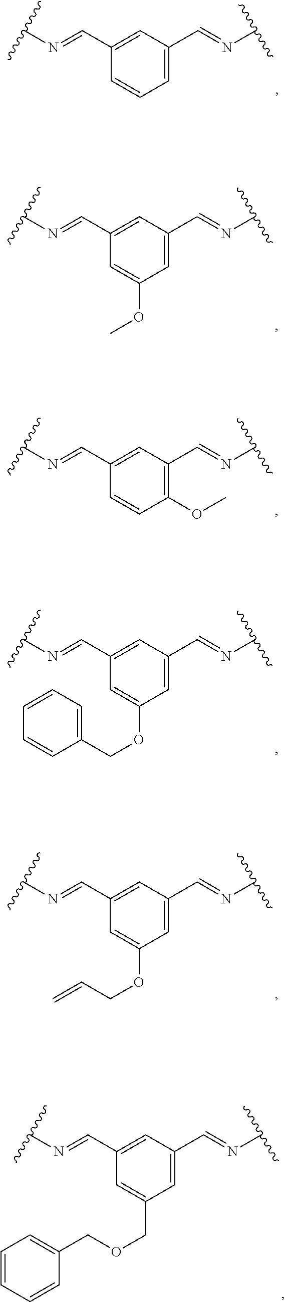 [SMILES: */N=C/c1cccc(/C=N/*)c1 ; */N=C/c1cc(/C=N/*)cc(OC)c1 ; */N=C/c1ccc(OC)c(/C=N/*)c1 ; */N=C/c1cc(/C=N/*)cc(OCc2ccccc2)c1 ; */N=C/c1cc(/C=N/*)cc(OCC=C)c1 ; */N=C/c1cc(/C=N/*)cc(COCc2ccccc2)c1]

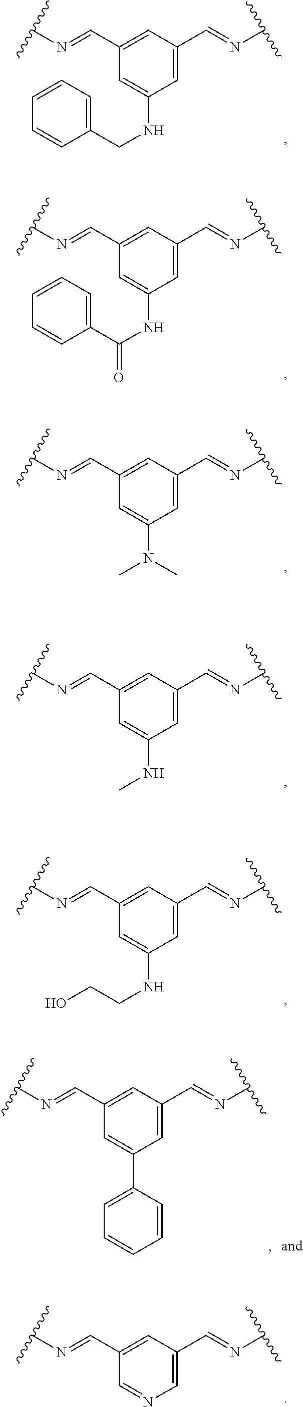

Some embodiments disclosed herein provide a compound of Formula II, wherein A⁶ can be selected from the group consisting of phenyl, naphthyl, benzo[d][1,3]dioxolyl, indolyl, and benzo[d]imidazolyl, each optionally substituted with one or more substituents selected from the group consisting of R¹¹ and R¹²; each R¹¹ can be separately selected from the group consisting of phenyl, pyrrolyl, and imidazolyl, each optionally substituted with a substituent selected from C₁-C₆ alkyl, C₁-C₆ alkoxy, C₁-C₆ alkylHN— and (C₁-C₆ alkyl)₂N—; each R¹² can be separately selected from the group consisting of bromo, chloro, fluoro, —(CH₂)ₘOR^{A}, and —NR^{B}R^{C}, where each R^{A} in the definition of R¹² can be separately selected from the group consisting of hydrogen and C₁-C₆ alkyl; each —NR^{B}R^{C} can be separately selected, wherein R^{B} and R^{C} can each be independently selected from the group consisting of hydrogen, —C(═O)R^{H}, C₁-C₆ alkyl optionally substituted with up to 5 fluoro, and an optionally substituted C₃-C₇ cycloalkyl further optionally fused with phenyl; or —NR^{B}R^{C} can be a morpholinyl, piperazinyl, pyrrolidinyl, and piperidinyl, each optionally substituted with one or more oxo; each R^{H} can be separately selected from the group consisting aryl and heteroaryl, each optionally substituted with one or more substituents each separately selected from the group consisting of halogen, C₁-C₆ alkyl optionally substituted with up to 5 fluoro, and C₁-C₆ alkoxy optionally substituted with up to 5 fluoro; G⁶ can be selected from the group consisting of: phenyl, naphthyl, benzo[d][1,3]dioxolyl, indolyl, and benzo[d]imidazolyl, each optionally substituted with one or more substituents selected from the group consisting of R¹⁴ and R¹⁵; each R¹⁴ can be separately selected from the group consisting of phenyl, pyrrolyl, and imidazolyl, each optionally substituted with a substituent selected from C₁-C₆ alkyl, C₁-C₆ alkoxy, C₁-C₆ alkylHN— and (C₁-C₆ alkyl)₂N—; and each R¹⁵ can be separately selected from the group consisting of bromo, chloro, fluoro, —(CH₂)ₘOR^{A}, and —NR^{B}R^{C}, where each R^{A} in the definition of R¹⁵ can be separately selected from the group consisting of hydrogen and C₁-C₆ alkyl.

Some embodiments disclosed herein provide a compound of Formula III:

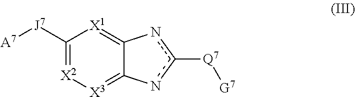

and pharmaceutically acceptable salts, esters stereoisomers, tautomers or prodrugs thereof;

wherein:

A⁷ is selected from the group consisting of aryl, heteroaryl, isoindolinyl, indenyl, dihydroindenyl, tetrahydroisoquinolinyl, and tetrahydronaphthalenyl, each optionally substituted with one or more substituents selected from the group consisting of R²¹, R²², and R²³, said aryl and heteroaryl in the definition of A⁷ are each further optionally fused with an optionally substituted nonaromatic heterocycle or an optionally substituted nonaromatic carbocycle; or A⁷ is C₃-C₇ cycloalkyl optionally substituted with one or more substituents selected from the group consisting of R²¹, R²², and R²³, said C₃-C₇ cycloalkyl in the definition of A⁷ is fused with an optionally substituted aryl or optionally substituted heteroaryl;

each R²¹ is independently selected from the group consisting of halogen, cyano, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted aryl, an optionally substituted heteroaryl, and an optionally substituted heterocycle;

each R²² is independently selected from the group consisting of —(CH₂)ₘOR^{A}, —O(CH₂)ₘOR^{A}, —C(═O)OR^{A}, —O(CH₂)ₒNR^{B}R^{C}, —(CH₂)ₘNR^{B}R^{C}, —C(═O)NR^{B}R^{C}, and —(CH₂)ₘSR^{A};

each R²³ is independently selected from the group consisting of phenyl, —NHC(═NH)NH₂, —(CH₂)ₘOR^{D}, —C(═NNR^{B}R^{C}C)H, —NR^{L}C(═O)NR^{B}R^{C}, —C(═O)NR^{D}N(═CHR^{G}), —(CH₂)ₘS(O)₀₋₂R^{D}, —(CH₂)ₘNO₂, —(CH₂)ₘCN, and —(CH₂)ₘR^{G} said phenyl in the definition of R²³ is substituted with one or more substituents selected from the group consisting of halogen, cyano, C₁-C₃ alkyl, an optionally substituted C₁-C₃ alkoxy, —O(CH₂)ₘOR^{A}, —(CH₂)ₘNR^{B}R^{C};

J⁷ is selected from the group consisting of —(CH₂)ₙ[NHC(═O)](CH₂)ₒ[NHC(═O)](CH₂)ₚ—, —(CH₂)ₙ[NHC(═O)](CH₂)ₒ[NH]_{q}—, —NH[C(═O)][C(═O)]NH— and

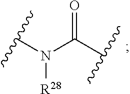

Q⁷ is selected from the group consisting of O (oxygen), —NR²⁸—, aryl, and arylamido; or Q⁷ is null;

each R²⁸ is independently selected from the group consisting of hydrogen and an optionally substituted C₁-C₄ alkyl;

G⁷ is selected from the group consisting aryl, heteroaryl, and heterocycle, each optionally substituted with one or more substituents selected from the group consisting of R²⁴, R²⁵, and R²⁶, said aryl and heteroaryl in the definition of G⁷ are each further optionally fused with an optionally substituted nonaromatic heterocycle or an optionally substituted nonaromatic carbocycle;

each R²⁴ is independently selected from the group consisting of halogen, cyano, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted aryl, an optionally substituted heteroaryl, and an optionally substituted heterocycle;

each R²⁵ is independently selected from the group consisting of —(CH₂)ₘOR^{A}, —O(CH₂)ₘOR^{A}, —C(═O)OR^{A}, —O(CH₂)ₒNR^{B}R^{C}, —(CH₂)ₘNR^{B}R^{C}, —(CH₂)ₘC(═O)NR^{B}R^{C}, —C(═NNR^{B}R^{C})H, and —(CH₂)ₘSR^{A};

each R²⁶ is independently selected from the group consisting of phenyl, —NHC(═NH)NH₂, —(CH₂)ₘOR^{D}, —C(═NNR^{B}R^{C})H, —NR^{L}C(═O)NR^{B}R^{C}, —C(═O)NR^{D}N(═CH)R^{G}, —(CH₂)ₘS(O)₀₋₂R^{D}, —(CH₂)ₘNO₂, —(CH₂)ₘCN, —(CH₂)ₘR^{G}, said phenyl in the definition of R²³ is substituted with —(CH₂)ₘNR^{B}R^{C};

X¹, X², and X³ are each independently selected from N (nitrogen) and CR²⁷

R²⁷ is selected from the group consisting of hydrogen, halogen, and an optionally substituted C₁-C₄ alkyl;

R^{A} is selected from the group consisting of hydrogen, C₁-C₆ alkyl, C₂-C₄ alkenyl, C₂-C₄ alkynyl, C₃-C₇ cycloalkyl, and C₁-C₆ haloalkyl;

each —NR^{B}R^{C} is separately selected, wherein R^{B} and R^{C} are each independently selected from the group consisting of hydrogen, —SO₂R^{H}, —C(═O)R^{H}, —(CH₂)ₙOR^{H}, —(CH₂)ₘR^{I}, —(CH₂)ₘR^{J}, —(CH₂)ₙC(═O)NR^{E}R^{F}, —(CH₂)ₙNR^{E}R^{F}, —SO₂NR^{E}R^{F}, heterocycle, C₁-C₆ alkyl, C₁-C₆ haloalkyl, C₁-C₆ heteroalkyl, C₃-C₇ cycloalkyl, and C₁-C₆ heterohaloalkyl where the C₃-C₇ cycloalkyl and the heterocycle are each optionally fused with an optionally substituted aryl or optionally substituted heteroaryl; or —NR^{B}R^{C} is an optionally substituted non-aromatic heterocycle linked through a ring nitrogen atom, said optionally substituted non-aromatic heterocycle is optionally fused with an optionally substituted aryl or optionally substituted heteroaryl;

each R^{D} is independently selected from the group consisting of hydrogen, an optionally substituted C₁-C₆ alkyl, an optionally substituted C₁-C₆ haloalkyl, an optionally substituted C₁-C₆ heteroalkyl, and —(CH₂)ₘR^{I};

each —NR^{E}R^{F} is separately selected, wherein R^{E} and R^{F} are each independently selected from the group consisting of hydrogen, an optionally substituted C₁-C₆ alkyl, an optionally substituted C₁-C₆ haloalkyl, an optionally substituted C₁-C₆ heteroalkyl, an optionally substituted C₃-C₇ cycloalkyl, an optionally substituted heterocycle, and —(CH₂)ₘR^{G}; or —NR^{E}R^{F} is an optionally substituted non-aromatic heterocycle linked through a ring nitrogen atom; or —NR^{E}R^{F} is C₁-C₆ alkylideneamino substituted with an optionally substituted aryl;

each R^{G} is independently selected from an optionally substituted aryl and an optionally substituted heteroaryl;

each R^{H} is independently selected from the group consisting of hydrogen, C₁-C₃ alkoxy, C₁-C₃ alkyl, C₁-C₃ haloalkyl, an optionally substituted aryl and an optionally substituted heteroaryl;

each R^{I} is independently selected from the group consisting of an optionally substituted aryl and an optionally substituted heteroaryl;

each R^{J} is independently selected from the group consisting of aryl and heteroaryl, each substituted with one or more —NR^{E}R^{F};

each R^{L} is independently selected from the group consisting of C₃-C₇ cycloalkyl, optionally substituted C₁-C₆ alkyl, optionally substituted C₁-C₆ alkoxy, —(CH₂)ₘOR^{LA}, —(CH₂)ₘNR^{LB}R^{LC}, aryl and heteroaryl, said aryl and heteroaryl in the definition of R^{L} are each independently optionally substituted with one or more substituents selected from the group consisting of halo, C₁-C₆ alkyl, C₁-C₆ haloalkyl, C₁-C₆ alkoxy, —(CH₂)ₘNR^{LD}R^{LE}, aryl and heteroaryl, said aryl and heteroaryl substituent off of R^{L} are each optionally substituted with one or more halo, C₁-C₆ alkyl, C₁-C₆ haloalkyl, C₁-C₆ alkoxy, or —(CH₂)ₘNR^{LF}R^{LG};

each R^{LA} is independently selected from the group consisting of hydrogen, C₁-C₆ alkyl, and C₁-C₆ haloalkyl;

R^{LB} and R^{LC} are each independently selected from the group consisting of hydrogen, C₁-C₆ alkyl, C₁-C₆ haloalkyl, and C₁-C₆ heteroalkenyl; or —NR^{LB}R^{LC} is an optionally substituted non-aromatic heterocycle linked through a ring nitrogen atom;

each —NR^{LD}R^{LE} is separately selected, wherein R^{LD} and R^{LE} are each independently selected from the group consisting of hydrogen, aryl, heteroaryl, and optionally substituted C₁-C₆ alkyl, said aryl and heteroaryl in the definition of R^{LD} and R^{LE} are each optionally substituted with C₁-C₆ alkyl or C₁-C₆ alkoxy; or —NR^{LD}R^{LE} is an optionally substituted non-aromatic heterocycle linked through a ring nitrogen atom;

each —NR^{LF}R^{LG} is separately selected, wherein R^{LF} and R^{LG} are each independently selected from the group consisting of hydrogen, and C₁-C₆ alkyl; or —NR^{LF}R^{LG} is an optionally substituted non-aromatic heterocycle linked through a ring nitrogen atom;

each m is independently 0, 1, or 2;

each n is independently 0, 1, 2, 3, or 4;

each o is independently 1, 2, or 3;

each p is independently 0, 1, 2, or 3;

each q is independently 0 or 1; and

any bond represented by a dashed and solid line represents a bond selected from the group consisting of a single bond and a double bond.

Some embodiments disclosed herein provide a compound of Formula III, wherein A⁷ can be selected from the group consisting of phenyl, pyridinyl, pyrimidinyl, imidazolyl, isoxazolyl, thienyl, indolyl, and benzimidazolyl, each substituted with one or more substituents selected from the group consisting of R²¹, R²², and R²³, said aryl and heteroaryl in the definition of A⁷ can each be further optionally fused with an optionally substituted nonaromatic heterocycle or an optionally substituted nonaromatic carbocycle; G⁷ can be selected from the group consisting aryl, heteroaryl, and heterocycle, each substituted with one or more substituents selected from the group consisting of R²⁴, R²⁵, and R²⁶, said aryl and heteroaryl in the definition of G⁷ can each be further optionally fused with an optionally substituted nonaromatic heterocycle or an optionally substituted nonaromatic carbocycle; R²¹ can be selected from the group consisting of fluorine and chlorine; R²² can be selected from the group consisting of —(CH₂)ₘOR^{A}, —C(═O)NR^{B}R^{C}, —NR^{B}R^{C}, and —(CH₂)ₘSR^{A}; R²³ can be selected from the group consisting of —(CH₂)ₘOR^{D}, and —C(═O)NR^{D}N(═CHR^{G}); R²⁵ can be selected from the group consisting of —(CH₂)ₘOR^{A}, —C(═O)NR^{B}R^{C}, —NR^{B}R^{C}; each R^{A} can be independently selected from the group consisting of hydrogen, C₁-C₆ alkyl, C₁-C₆ haloalkyl, and C₁-C₆ heteroalkyl; each —NR^{B}R^{C} can be separately selected, wherein R^{B} and R^{C} can each be independently selected from the group consisting of C₁-C₆ alkyl, C₁-C₆ haloalkyl, C₁-C₆ heteroalkyl, and C₁-C₆ heterohaloalkyl; or —NR^{B}R^{C} can be an optionally substituted non-aromatic heterocycle linked through a ring nitrogen atom; or —NR^{B}R^{C} is an optionally substituted C₁-C₆ alkylideneamino; J⁷ can be

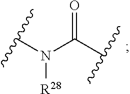

each R²⁸ can be independently selected from the group consisting of hydrogen and an optionally substituted C₁-C₃ alkyl, with the proviso when A⁷ and G⁷ are a phenyl then at least one of R²², R²³, R²⁵, and R²⁶ can be selected from the group consisting of —(CH₂)ₘOR^{A}, —(CH₂)ₘOR^{D}, —NR^{B}R^{C}, —C(═O)NR^{D}N(═CHR^{G}), —NR^{E}R^{F}, —C(═O)NR^{B}R^{C}, and an optionally substituted phenyl.

Some embodiments disclosed herein provide a compound of Formula III, wherein A⁷ can be selected from the group consisting of phenyl, pyridinyl, pyrimidinyl, imidazolyl, isoxazolyl, thienyl, indolyl, and benzimidazolyl, each substituted with one or more substituents selected from the group consisting of R²¹, R²², and R²³ said aryl and heteroaryl in the definition of A⁷ can each be further optionally fused with an optionally substituted nonaromatic heterocycle or an optionally substituted nonaromatic carbocycle; G⁷ can be selected from the group consisting aryl, heteroaryl, and heterocycle, each substituted with one or more substituents selected from the group consisting of R²⁴, R²⁵, and R²⁶, said aryl and heteroaryl in the definition of G⁷ can each be further optionally fused with an optionally substituted nonaromatic heterocycle or an optionally substituted nonaromatic carbocycle; R²¹ can be selected from the group consisting of C₁-C₆ alkyl, fluorine, and chlorine; R²² can be selected from the group consisting of —NHC(═O)R^{E}R^{F}, —(CH₂)ₘOR^{A}, and —NR^{B}R^{C}; R²³ can be selected from the group consisting of —(CH₂)ₘOR^{D}, and —C(═O)NR^{D}N(═CHR^{G}); R^{A} can be selected from the group consisting of hydrogen, C₁-C₆ alkyl, and C₁-C₆ haloalkyl; each —NR^{B}R^{C} can be separately selected, wherein R^{B} and R^{C} can each be independently selected from the group consisting of hydrogen, C₁-C₆ alkyl, C₁-C₆ haloalkyl, C₁-C₆ heteroalkyl, and C₁-C₆ heterohaloalkyl; or —NR^{B}R^{C} can be an optionally substituted non-aromatic heterocycle linked through a ring nitrogen atom; or —NR^{B}R^{C} can be an optionally substituted C₁-C₆ alkylideneaminyl; and J⁷ can be

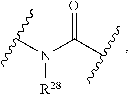

with the proviso when A⁷ and G⁷ are a phenyl then at least one of R²², R²³, R²⁵, and R²⁶ can be selected from the group consisting of —(CH₂)ₘOR^{A}, —(CH₂)ₘOR^{D}, —NR^{B}R^{C}, —NHC(═O)R^{E}R^{F}, —C(═O)NR^{D}N(═CHR^{G}), and an optionally substituted phenyl.

Some embodiments disclosed herein provide a compound of Formula III having the structure of Formula IIIaa:

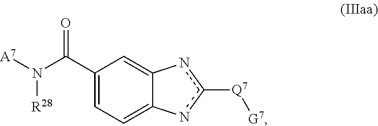

and pharmaceutically acceptable salts, esters, stereoisomers, tautomers or prodrugs thereof, or having the formula IIIab:

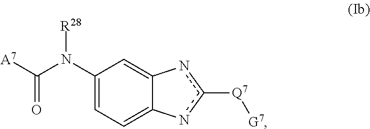

and pharmaceutically acceptable salts, esters, stereoisomers, tautomers or prodrugs thereof.

Some embodiments disclosed herein provide a compound of Formula IIIab having the structure of Formula IIIabb:

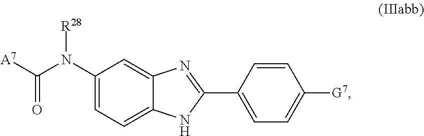

and pharmaceutically acceptable salts, esters, stereoisomers, tautomers or prodrugs thereof, wherein A⁷ can be aryl substituted with one or more substituents selected from the group consisting of R²¹, R²², and R²³ each R²¹ can be independently selected from the group consisting of halogen, cyano, C₁-C₆ alkyl, and C₁-C₆ alkoxy; each R²² can be independently selected from the group consisting of —(CH₂)ₘOR^{A}, —O(CH₂)ₘOR^{A} and —(CH₂)ₘNR^{B}R^{C}; each R²³ can be phenyl substituted with —(CH₂)ₘNR^{B}R^{C}; G⁷ can be heterocycle substituted with one or more substituents selected from the group consisting of R²⁴, R²⁵, and R²⁶; each R²⁴ can be independently selected from the group consisting of halogen, cyano, C₁-C₆ alkyl, and C₁-C₆ alkoxy; each R²⁵ can be independently selected from the group consisting of —(CH₂)ₘOR^{A}, —O(CH₂)ₘOR^{A}; each R²⁶ can be phenyl substituted with one or more —(CH₂)ₘNR^{B}R^{A}; R^{B} can be hydrogen; R^{C} can be —C(═O)R^{H}; and R^{H} can be an optionally substituted aryl.

Some embodiments disclosed herein provide a compound of Formula IIIabb, wherein A⁷ can be phenyl substituted with one or more substituents selected from the group consisting of R²²; each R²² can be independently selected from the group consisting of —(CH₂)ₘOR^{A}, and —O(CH₂)ₘOR^{A}; each R^{A} can be independently selected from the group consisting of hydrogen, C₁-C₃ alkyl, and C₁-C₃ haloalkyl; G⁷ can be piperidinyl substituted with one or more substituents selected from the group consisting of R²⁶; each R²⁶ can be phenyl substituted with —NHC(═O)R^{H}; and R^{H} can be an optionally substituted phenyl.

Some embodiments disclosed herein provide a compound of Formula III having the structure of Formula IIIb:

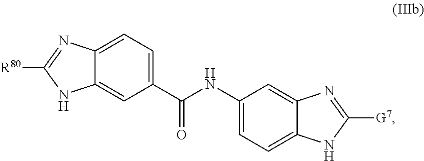

and pharmaceutically acceptable salts thereof, wherein R⁸⁰ can be selected from the group consisting of hydrogen, R²¹, R²², and R²³.

Some embodiments disclosed herein provide a compound of Formula III having the structure of Formula IIIc:

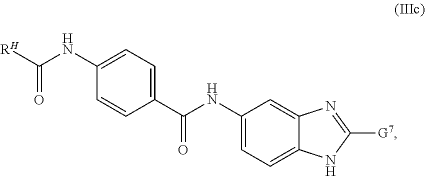

and pharmaceutically acceptable salts thereof.

Some embodiments disclosed herein provide a compound of Formula III having the structure of Formula IIId:

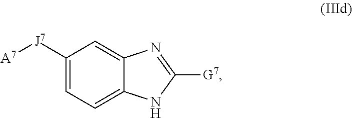

and pharmaceutically acceptable salts thereof, wherein A⁷ can be selected from the group consisting of aryl and heteroaryl, each substituted with one or more substituents selected from the group consisting of R²¹, R²², and R²³, said aryl and heteroaryl in the definition of A⁷ can each be further optionally fused with an optionally substituted nonaromatic heterocycle or an optionally substituted nonaromatic carbocycle; or A⁷ can be C₃-C₇ cycloalkyl optionally substituted with one or more substituents selected from the group consisting of R²¹, R²², and R²³, said C₃-C₇ cycloalkyl in the definition of A⁷ can be fused with an optionally substituted aryl or optionally substituted heteroaryl; J⁷ can be selected from the group consisting of —(CH₂)ₙ[NHC(═O)](CH₂)ₒNHC(═O)(CH₂)ₚ—, —(CH₂)ₙ[NHC(═O)](CH₂)[NH]_{q}—, and

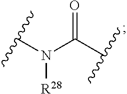

and G⁷ can be selected from the group consisting of a aryl and heteroaryl, each substituted with one or more substituents selected from the group consisting of R²⁴, R²⁵, and R²⁶, said aryl and heteroaryl in the definition of G⁷ can each be further optionally fused with an optionally substituted nonaromatic heterocycle or an optionally substituted nonaromatic carbocycle.

Some embodiments disclosed herein provide a compound of Formula IIId having the structure of Formula IIIda:

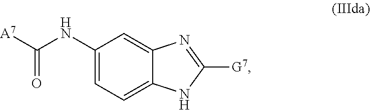

and pharmaceutically acceptable salts, esters, or prodrugs thereof, wherein A⁷ can be selected from the group consisting of phenyl, indolyl, pyridinyl, pyrimidinyl, thienyl, benzothiofuranyl, naphthalenyl, and tetrahydronaphthalenyl, each substituted with one or more substituents selected from the group consisting of R²¹, R²², and R²³; and G⁷ can be selected from the group consisting of a aryl and heteroaryl, each optionally substituted with one or more substituents selected from the group consisting of R²⁴, R²⁵, and R²⁶ said aryl and heteroaryl in the definition of G⁷ can each be further optionally fused with an optionally substituted nonaromatic heterocycle or an optionally substituted nonaromatic carbocycle.

Some embodiments disclosed herein provide a compound of Formula IIId having the structure of Formula IIIdb:

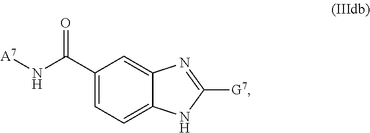

and pharmaceutically acceptable salts, esters, or prodrugs thereof, wherein A⁷ can be selected from the group consisting of phenyl, indolyl, pyridinyl, pyrimidinyl, thienyl, benzothiofuranyl, naphthalenyl, and tetrahydronaphthalenyl, each substituted with one or more substituents selected from the group consisting of R²¹, R²², and R²³; and G⁷ can be selected from the group consisting of a aryl and heteroaryl, each optionally substituted with one or more substituents selected from the group consisting of R²⁴, R²⁵, and R²⁶ said aryl and heteroaryl in the definition of G⁷ can each be further optionally fused with an optionally substituted nonaromatic heterocycle or an optionally substituted nonaromatic carbocycle.

Some embodiments disclosed herein provide a compound of Formula IIIda or Formula IIIdb, wherein G⁷ can be selected from the group consisting of phenyl, naphthyl, indolyl, dihydrobenzofuranyl, 1,4-benzodioxanyl, benzotriazolyl, benzimidazolyl, benzofuranyl, and 2,1,3-benzoxadiazolyl, each optionally substituted with one or more substituents selected from the group consisting of R²⁴, R²⁵, and R²⁶. In some embodiments, R²¹ can be selected from the group consisting of halogen, an optionally substituted C₁-C₆ alkyl, an optionally substituted C₁-C₆ alkoxy, and an optionally substituted C₃-C₇ cycloalkyl; R²² can be selected from the group consisting of —(CH₂)ₘOR^{A}, —O(CH₂)ₘOR^{A}, and —NR^{B}R^{C}; R²³ can be selected from the group consisting of —(CH₂)ₘOR^{D}, —(CH₂)ₘS(O)₀₋₂R^{D}, and —(CH₂)ₘR^{G}; R²⁴ can be selected from the group consisting of halogen, an optionally substituted C₁-C₆ alkyl, an optionally substituted C₁-C₆ alkoxy, an optionally substituted C₃-C₇ cycloalkyl; R²⁵ can be selected from the group consisting of —(CH₂)ₘOR^{A}, —O(CH₂)ₘOR^{A}, —C(═O)NR^{B}R^{C}, —C(═NNR^{B}R^{C})H, —NR^{B}R^{C}; R²⁶ can be selected from the group consisting of —(CH₂)ₘOR^{D}, —(CH₂)ₘR^{G}; R^{A} can be selected from the group consisting of hydrogen, C₁-C₆ alkyl, C₃-C₇ cycloalkyl, and C₁-C₆ haloalkyl; each —NR^{B}R^{C} can be separately selected, wherein R^{B} and R^{C} can each be independently selected from the group consisting of hydrogen, —SO₂R^{H}, —C(═O)R^{H}, —(CH₂)ₙOR^{H}, —(CH₂)ₘR^{I}, —(CH₂)ₘR^{J}, —(CH₂)ₙC(═O)NR^{E}R^{F}, —(CH₂)ₙNR^{E}R^{F}, —SO₂NR^{E}R^{F}, C₁-C₆ alkyl, C₁-C₆ haloalkyl, C₁-C₆ heteroalkyl, C₃-C₇ cycloalkyl, and C₁-C₆ heterohaloalkyl where the alkyl and the heteroalkyl are optionally fused with an aryl or heteroaryl; or —NR^{B}R^{C} can be an optionally substituted heterocyle; each R^{D} can be independently selected from the group consisting of hydrogen, an optionally substituted C₁-C₆ alkyl, and —(CH₂)ₘR^{I}; each —NR^{E}R^{F} can be separately selected, wherein R^{E} and R^{F} can each be independently selected from the group consisting of hydrogen, an optionally substituted C₁-C₆ alkyl, and —(CH₂)ₘR^{G}; or —NR^{E}R^{F} can be an optionally substituted C₁-C₆ alkylideneamino; or —NR^{E}R^{F} can be an optionally substituted heterocyle; each R^{G} can be independently selected from the group consisting of an optionally substituted aryl and an optionally substituted heteroaryl; each R^{H} can be independently selected from the group consisting of hydrogen, C₁-C₃ alkoxy, C₁-C₃ alkyl, C₁-C₃ haloalkyl, an optionally substituted aryl and optionally substituted heteroaryl; each R^{I} can be independently selected from the group consisting of an optionally substituted aryl and an optionally substituted heteroaryl; each R^{J} can be independently selected from the group consisting of aryl and heteroaryl, each substituted with one or more —NR^{E}R^{F}; each m can be independently 0, 1, or 2; and each n can be independently 0, 1, 2, 3, or 4.

Some embodiments disclosed herein provide a compound of Formula III having the proviso that a compound of Formula III is not selected from the group consisting of:

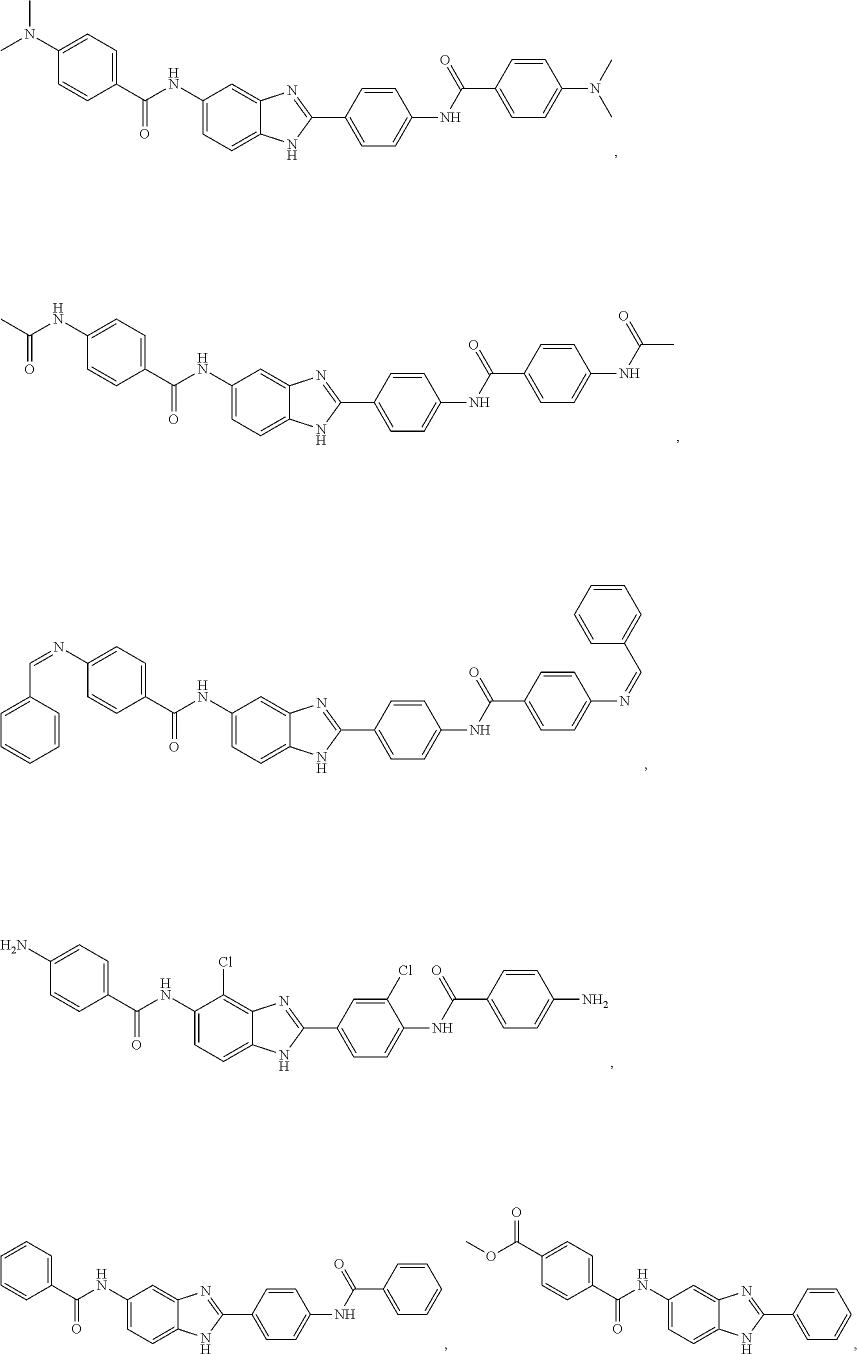

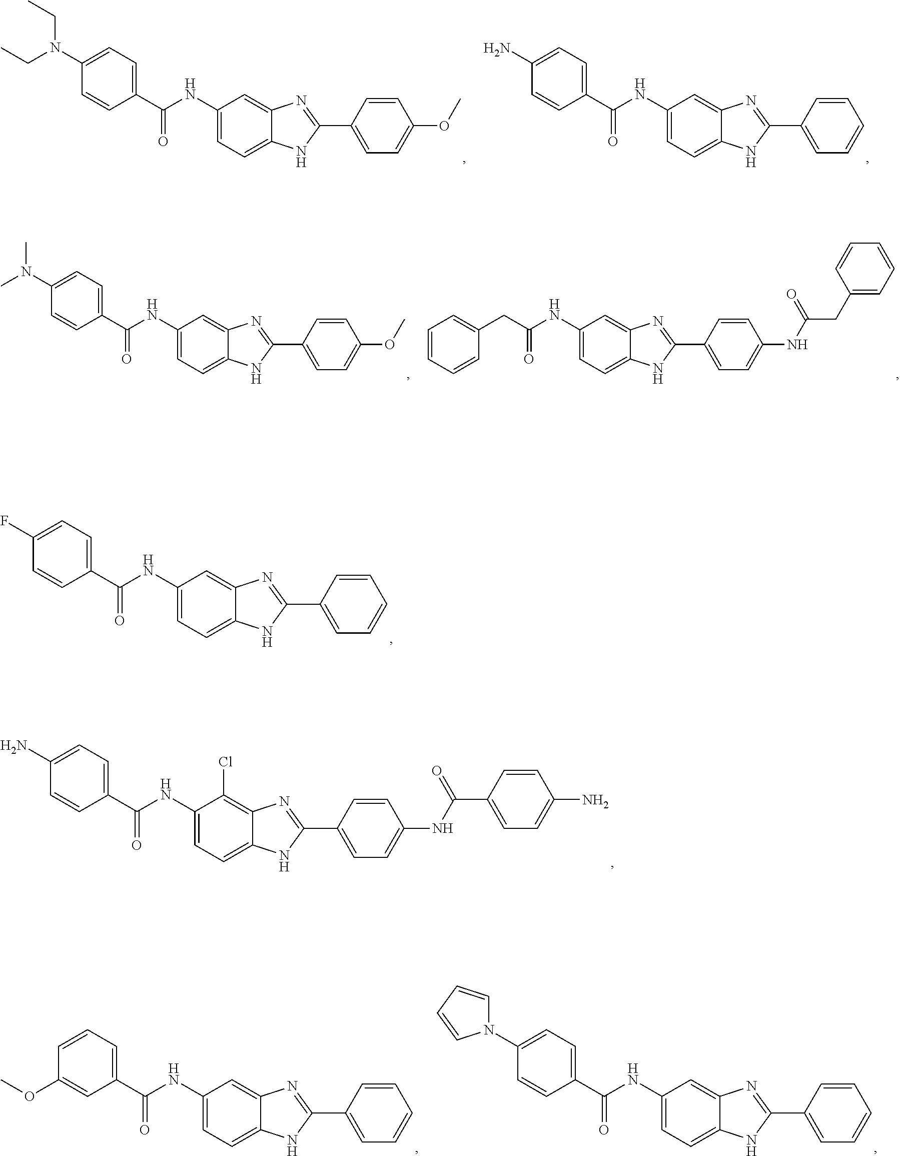

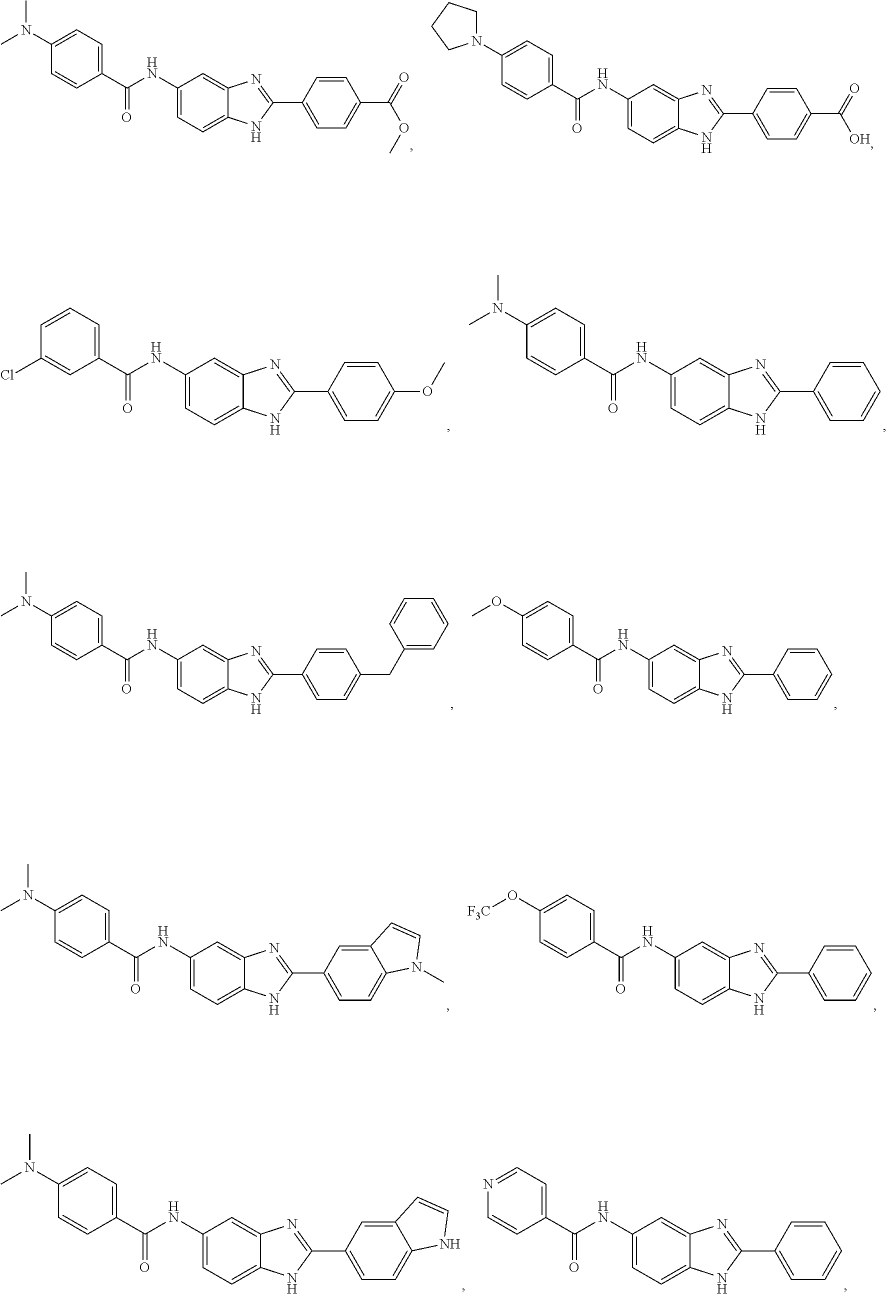

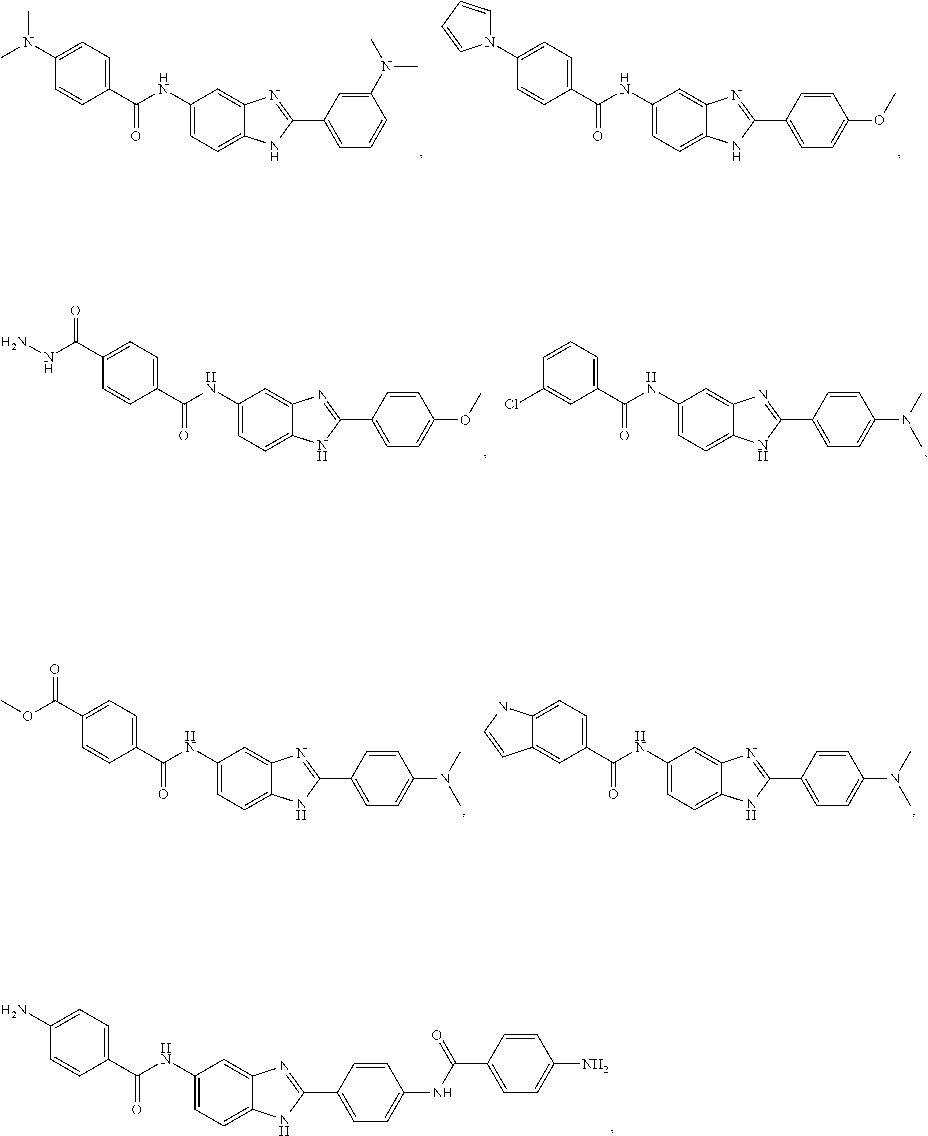

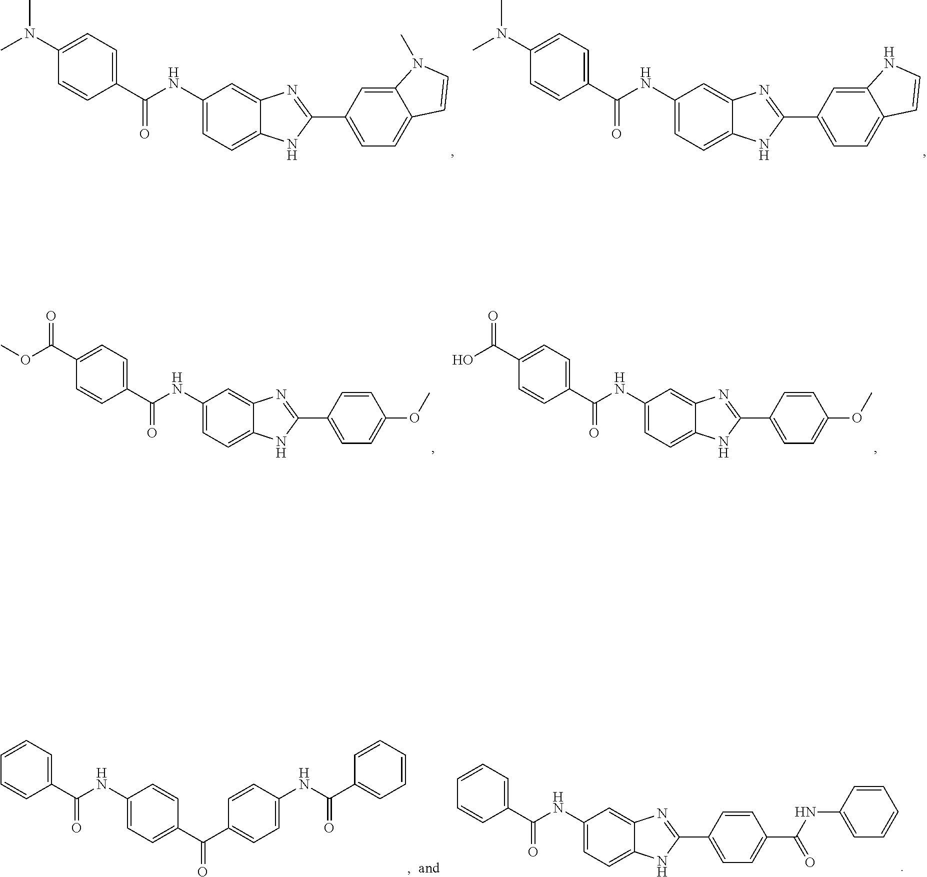

Some embodiments disclosed herein provide a compound of Formula IV:

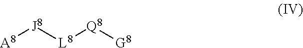

and pharmaceutically acceptable salts, esters stereoisomers, tautomers or prodrugs thereof;

wherein:

A⁸ is selected from the group consisting of heterocycle, aryl and heteroaryl, each optionally substituted with one or more substituents selected from the group consisting of R³¹, R³², and R³³, said aryl and heteroaryl in the definition of A⁸ are each further optionally fused with an optionally substituted nonaromatic heterocycle or an optionally substituted nonaromatic carbocycle;

G⁸ is selected from the group consisting of aryl and heteroaryl, each optionally substituted with one or more substituents selected from the group consisting of R³⁴, R³⁵, and R³⁶, said aryl and heteroaryl in the definition of G⁸ are each further optionally fused with an optionally substituted nonaromatic heterocycle or an optionally substituted nonaromatic carbocycle;

J⁸ is selected from the group consisting of aryl, heteroaryl, heterocycle, —C(═O)—, —(CH═CH)—, —OC(═O)—, —NHC(═O)NH—, —NHC(═S)NH—, —S(═O)₂—NH₂—, —OC(═S)—, —NHC(═S)—, —(CH₂)ₙNH—, —(CH₂)ₙ[NHC(═O)](CH₂)ₒNHC(═O)(CH₂)ₚ—, —CH₂)ₙ[NHC(═O)](CH₂)ₒ[NH]_{q},

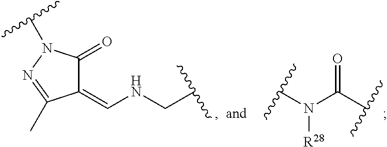

or J⁸ is null;

Q⁸ is selected from the group consisting of aryl, heteroaryl, heterocycle, —C(═O)—, —(CH═CH)—, —OC(═O)—, —NHC(═O)NH—, —NHC(═S)NH—, —S(═O)₂—NH₂—, —OC(═S)—, —NHC(═S)—, —(CH₂)ₙNH—, —(CH₂)ₙ[NHC(═O)](CH₂)ₒNHC(═O)(CH₂)ₚ—, —(CH₂)ₙ[NHC(═O)](CH₂)ₒ[NH]_{q}—,

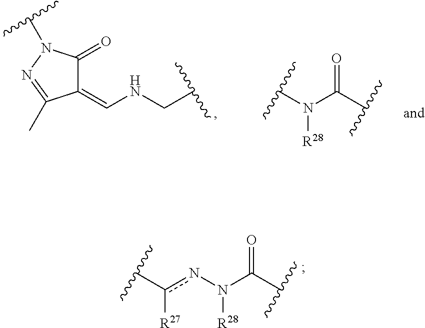

or Q⁸ is null;

L⁸ is selected from the group consisting of

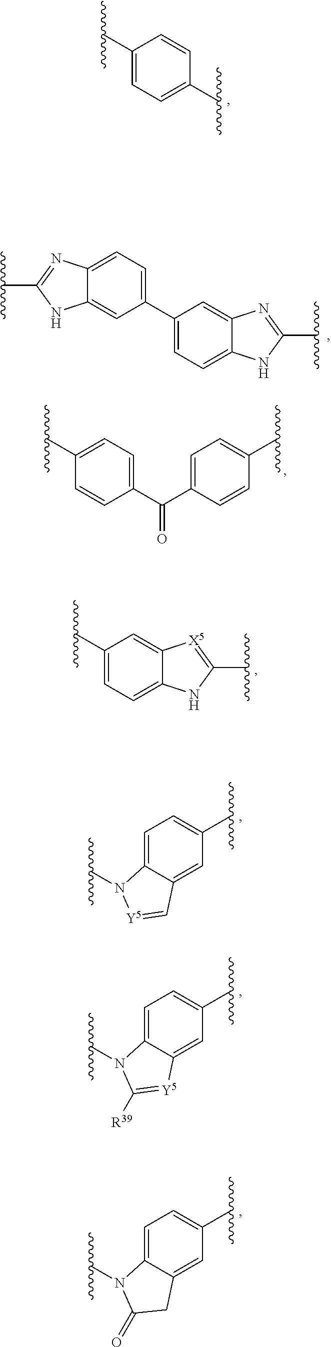

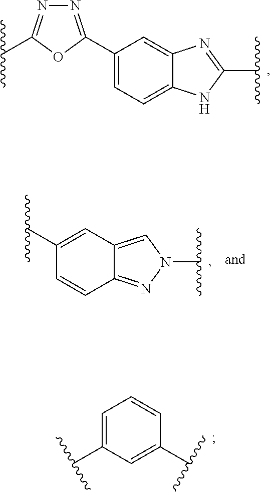 [SMILES: *c1nc2cc(-c3nnc(*)o3)ccc2[nH]1 ; *c1ccc2nn(*)cc2c1 ; *c1cccc(*)c1]

X⁵ is selected from the group consisting of N (nitrogen) and CR³⁹;

Y⁵ is selected from the group consisting of N (nitrogen) and CR⁴⁰;

each R²⁷ is independently selected from the group consisting of hydrogen, halogen, and an optionally substituted C₁-C₄ alkyl;

each R²⁸ is independently selected from the group consisting of hydrogen and an optionally substituted C₁-C₄ alkyl;

each R³¹ is independently selected from the group consisting of halogen, cyano, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₁-C₆ haloalkyl, and an optionally substituted C₁-C₆ heteroalkyl;

each R³² is independently selected from the group consisting of halogen, —(CH₂)ₘOR^{A}, —NR^{B}R^{C}, and —(CH₂)ₘSR^{A};

each R³³ is independently selected from the group consisting of halogen, —C(═O)OH, —(CH₂)ₘOR^{D}, —NR^{E}R^{F}, —NR^{L}C(═O)NR^{B}R^{C}, —(CH₂)ₘS(O)₀₋₂R^{D}, —(CH₂)ₘNO₂, —(CH₂)ₘCN, and —(CH₂)ₘR^{G};

each R³⁴ is independently selected from the group consisting of halogen, cyano, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₁-C₆ haloalkyl, and an optionally substituted C₁-C₆ heteroalkyl;

each R³⁵ is independently selected from the group consisting of halogen, C(═O)OH, —(CH₂)ₘOR^{A}, —NR^{B}R^{C}, —C(═O)NR^{B}R^{C}, and —(CH₂)ₘSR^{A};

each R³⁶ is independently selected from the group consisting of halogen, —(CH₂)ₘOR^{D}, —NR^{E}R^{F}, —NR^{L}C(═O)NR^{B}R^{C}, —(CH₂)ₘS(O)₀₋₂R^{D}, —(CH₂)ₘNO₂, —(CH₂)ₘCN, and —(CH₂)ₘR^{G};

each R³⁹ and R⁴⁰ are independently selected from the group consisting of hydrogen, halogen, —OH, —NHR^{B}, and an optionally substituted C₁-C₄ alkyl;

each R^{A} is independently selected from the group consisting of hydrogen, C₁-C₆ alkyl, C₁-C₆ haloalkyl, C₁-C₆ heteroalkyl, and C₁-C₆ heterohaloalkyl;

each —NR^{B}R^{C} is separately selected, wherein R^{B} and R^{C} are each independently selected from the group consisting of hydrogen, —SO₂R^{H}, —C(═O)R^{H}, —C(═O)C(═O)R^{H}, —(CH₂)ₘC(═O)OR^{H}, —C(═O)NR^{E}R^{F}, —(CH₂)ₘR^{G}, —(CH₂)ₘOR^{H}, —(CH₂)ₘR^{H}, C₁-C₆ alkyl, C₃-C₇ cycloalkyl, C₂-C₆ alkenyl, non-aromatic heterocycle, C₁-C₆ haloalkyl, C₁-C₆ heteroalkyl, and C₁-C₆ heterohaloalkyl where the C₃-C₇ cycloalkyl and the non-aromatic heterocycle are optionally fused with an an optionally substituted aryl or an optionally substituted heteroaryl; or —NR^{B}R^{C} is an optionally substituted non-aromatic heterocycle linked through a ring nitrogen atom; or —NR^{B}R^{C} is an optionally substituted C₁-C₆ alkylideneamino;

each R^{D} is independently selected from the group consisting of hydrogen, an optionally substituted C₁-C₆ alkyl, an optionally substituted C₁-C₆ haloalkyl, an optionally substituted C₁-C₆ heteroalkyl, and —(CH₂)ₘR^{G}

each —NR^{E}R^{F} is separately selected, wherein R^{E} and R^{F} are each independently selected from the group consisting of hydrogen, an optionally substituted C₁-C₆ alkyl, an optionally substituted C₁-C₆ haloalkyl, an optionally substituted C₁-C₆ heteroalkyl, and —(CH₂)ₘR^{G}; or —NR^{E}R^{F} is an optionally substituted C₁-C₆ alkylideneaminyl; or —NR^{E}R^{F} is an optionally substituted non-aromatic heterocycle linked through a ring nitrogen atom;

each R^{G} is independently selected from an optionally substituted aryl and an optionally substituted heteroaryl;

R^{H} is selected from the group consisting of hydrogen, C₁-C₃ alkyl, C₁-C₃ haloalkyl, C₃-C₇ cycloalkyl, and an optionally substituted aryl or an optionally substituted heteroaryl;

each R^{L} is independently selected from the group consisting of C₃-C₇ cycloalkyl, optionally substituted C₁-C₆ alkyl, optionally substituted C₁-C₆ alkoxy, —(CH₂)ₘOR^{LA}, —(CH₂)ₘNR^{LB}R^{LC}, aryl and heteroaryl, said aryl and heteroaryl in the definition of R^{L} are each independently optionally substituted with one or more substituents selected from the group consisting of halo, C₁-C₆ alkyl, C₁-C₆ haloalkyl, C₁-C₆ alkoxy, —(CH₂)ₘNR^{LD}R^{LE}, aryl and heteroaryl, said aryl and heteroaryl substituent off of R^{L} are each optionally substituted with one or more halo, C₁-C₆ alkyl, C₁-C₆ haloalkyl, C₁-C₆ alkoxy, or —(CH₂)ₘNR^{LF}R^{LG};

each R^{LA} is independently selected from the group consisting of hydrogen, C₁-C₆ alkyl, and C₁-C₆ haloalkyl;

R^{LB} and R^{LC} are each independently selected from the group consisting of hydrogen, C₁-C₆ alkyl, C₁-C₆ haloalkyl, and C₁-C₆ heteroalkenyl; or —NR^{LB}R^{LC} is an optionally substituted non-aromatic heterocycle linked through a ring nitrogen atom;

each —NR^{LD}R^{LE} is separately selected, wherein R^{LD} and R^{LE} are each independently selected from the group consisting of hydrogen, aryl, heteroaryl, and optionally substituted C₁-C₆ alkyl, said aryl and heteroaryl in the definition of R^{LD} and

R^{LE} are each optionally substituted with C₁-C₆ alkyl or C₁-C₆ alkoxy; or —NR^{LD}R^{LE} is an optionally substituted non-aromatic heterocycle linked through a ring nitrogen atom;

each —NR^{LF}R^{LG} is separately selected, wherein R^{LF} and R^{LG} are each independently selected from the group consisting of hydrogen, and C₁-C₆ alkyl; or —NR^{LF}R^{LG} is an optionally substituted non-aromatic heterocycle linked through a ring nitrogen atom;

each —NR^{LF}R^{LG} is separately selected, wherein R^{LF} and R^{LG} are each independently selected from the group consisting of hydrogen, and C₁-C₆ alkyl; or —NR^{LF}R^{LG} is an optionally substituted non-aromatic heterocycle linked through a ring nitrogen atom;

each m is independently 0, 1, or 2;

each n is independently 0, 1, 2, 3, or 4;

each o is independently 1, 2, or 3;

each p is independently 0, 1, 2, or 3;

each q is independently 0 or 1; and

any bond represented by a dashed and solid line represents a bond selected from the group consisting of a single bond and a double bond.

Some embodiments disclosed herein provide a compound of Formula IV, wherein J⁸ and Q⁸ can each be null. Some embodiments disclosed herein provide a compound of Formula IV, wherein A⁸ can be aryl substituted with one or more substituents selected from the group consisting of R³¹, R³², and R³³; J⁸ can be —NHC(═O)—; L⁸ can be

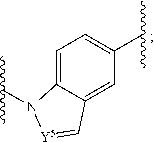

Q⁸ can be null; and G⁸ can be aryl substituted with one or more substituents selected from the group consisting of R³⁴, R³⁵, and R³⁶. Some embodiments disclosed herein provide a compound of Formula IV, wherein A⁸ can be aryl substituted with one or more R³²; J⁸ can be —C(═O)—; L⁸ can be

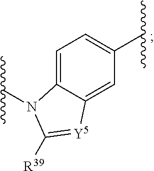

### Qs can be

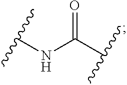 [SMILES: *NC(*)=O]

and G⁸ can be aryl substituted with one or more R³⁵. Some embodiments disclosed herein provide a compound of Formula IV, wherein A⁸ can be heteroaryl substituted with one or more substituents selected from the group consisting of R³¹, R³², and R³³; G⁸ can be heteroaryl substituted with one or more substituents selected from the group consisting of R³⁴, R³⁵, and R³⁶; J⁸ can be

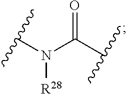

### and Q⁸ can be

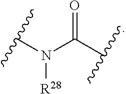

Some embodiments disclosed herein provide a compound of Formula IV, wherein L⁸ can be

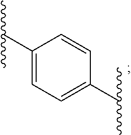 [SMILES: *c1ccc(*)cc1]

J⁸ can be —C(═O)—; Q⁸ can be C(═O)NH—; A⁸ can be aryl substituted with R³²; G⁸ can be aryl substituted with R³⁵; R³² can be —NR^{B}R^{C}, and R³⁵ can be —NR^{B}R^{C}.

Some embodiments disclosed herein provide a compound of Formula IV having the structure of Formula IVa:

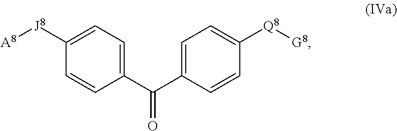

and pharmaceutically acceptable salts thereof, wherein J⁸ can be selected from the group consisting —OC(═O)—, —S(═O)₂—NH₂—, —(CH₂)ₙNH—, and

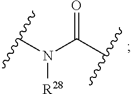

and Q⁸ can be selected from the group consisting —OC(═O)—, —S(═O)₂—NH₂—, —(CH₂)ₙNH—, and

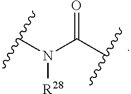

Some embodiments disclosed herein provide a compound of Formula IVa, wherein J⁸ can be selected from the group consisting of —S(═O)₂—NH₂— and

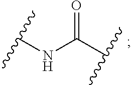 [SMILES: *NC(*)=O]

and Q⁸ can be selected from the group consisting —S(═O)₂—NH₂—, and

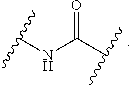 [SMILES: *NC(*)=O]

Some embodiments disclosed herein provide a compound of Formula IV having the structure of Formula IVb:

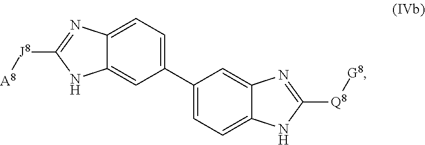

and pharmaceutically acceptable salts thereof, wherein J⁸ can be —CH═CH—; or J⁸ can be null; and Q⁸ can be —CH═CH—; or Q⁸ can be null.

Some embodiments disclosed herein provide a compound of Formula IVb having the structure of Formula IVba:

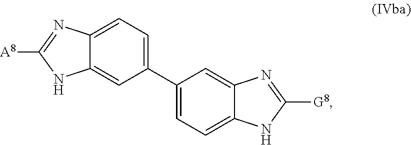

and pharmaceutically acceptable salts thereof.

Some embodiments disclosed herein provide a compound of Formula IVb having the structure of Formula IVbc:

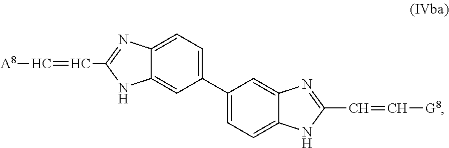

and pharmaceutically acceptable salts thereof.

Some embodiments disclosed herein provide a compound of Formula IV having the structure of Formula IVc:

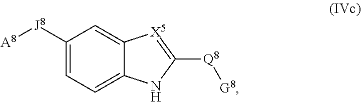

and pharmaceutically acceptable salts thereof, wherein J⁸ can be selected from the group consisting of —OC(═O)—, —NHC(═O)NH—, —S(═O)₂—NH₂—, and

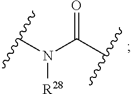

or can be null; Q⁸ can be selected from the group consisting of —OC(═O)—, —NHC(═O)NH—, —S(═O)₂—NH₂—, and

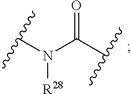

or Q can be null.

Some embodiments disclosed herein provide a compound of Formula IVc having the structure of Formula IVca:

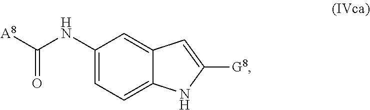

and pharmaceutically acceptable salts thereof, or having the structure of Formula IVcb:

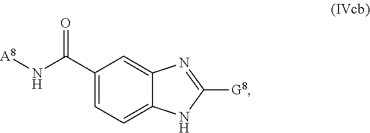

and pharmaceutically acceptable salts thereof, or having the structure of Formula IVcc:

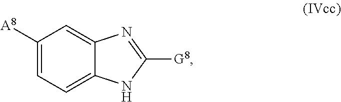

and pharmaceutically acceptable salts thereof.

Some embodiments disclosed herein provide a compound of Formula IV having the structure of Formula IVd:

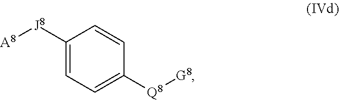

and pharmaceutically acceptable salts thereof, wherein J⁸ can be selected from the group consisting of —C(═O)—, —(CH₂)NH—, —NHC(═S)NH—, and

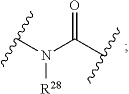

or J⁸ can be null; Q⁸ can be selected from the group consisting of —C(═O)—, —NHC(═S)NH—, and

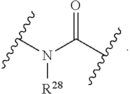

Some embodiments disclosed herein provide a compound of Formula IVd having the structure of Formula IVda:

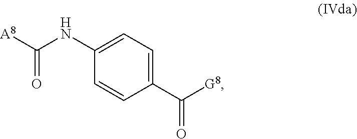

and pharmaceutically acceptable salts thereof, or having the structure of Formula IVdb:

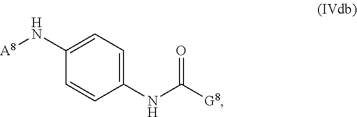

and pharmaceutically acceptable salts thereof, or having the structure of Formula IVdc:

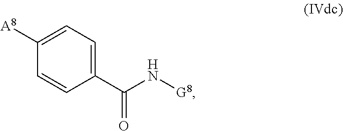

and pharmaceutically acceptable salts thereof, or having the structure of Formula IVde:

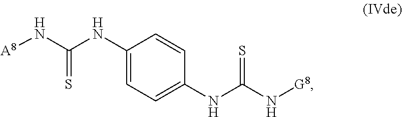

and pharmaceutically acceptable salts thereof.

Some embodiments disclosed herein provide a compound of Formula IV having the structure of Formula IVe:

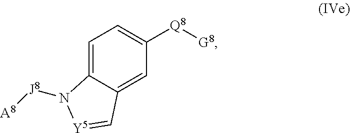

and pharmaceutically acceptable salts thereof, wherein J⁸ can be null; and Q⁸ can be

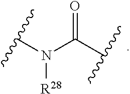

Some embodiments disclosed herein provide a compound of Formula IVe having the structure of Formula IVea:

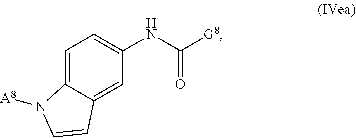

and pharmaceutically acceptable salts thereof, or having the structure of Formula IVeb:

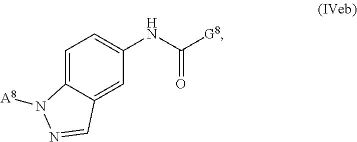

and pharmaceutically acceptable salts thereof, or having the structure of Formula IVec:

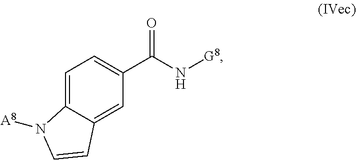

and pharmaceutically acceptable salts thereof.

Some embodiments disclosed herein provide a compound of Formula IV having the structure of Formula IVf:

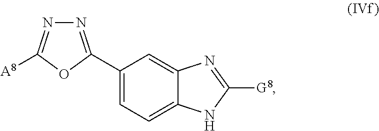

and pharmaceutically acceptable salts thereof.

Some embodiments disclosed herein provide a compound of Formula IV having the structure of Formula IVg:

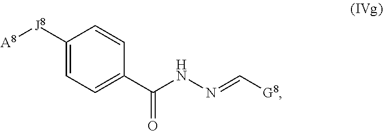

and pharmaceutically acceptable salts thereof, wherein J⁸ can be selected from the group consisting of —(CH₂)ₙ[NHC(═O)](CH₂)ₒNHC(═O)(CH₂)ₚ— and —(CH₂)ₙ[NHC(═O)](CH₂)ₒ[NH]_{q}—.

Some embodiments disclosed herein provide a compound of Formula IV having the structure of Formula IVh:

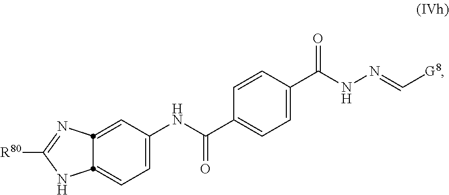

and pharmaceutically acceptable salts thereof, wherein R⁸⁰ can be selected from the group consisting of hydrogen, R³¹, R³², and R³³.

Some embodiments disclosed herein provide a compound of Formula IV having the structure of Formula IVi:

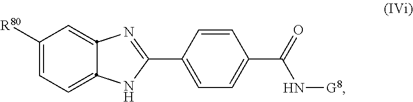

and pharmaceutically acceptable salts thereof, wherein R⁵⁸ can be selected from the group consisting of hydrogen, R³¹, R³², and R³³. Some embodiments disclosed herein provide a compound of Formula IVi, wherein G⁸ can be phenyl optionally substituted with one or more substituents selected from the group consisting of R³⁴, R³⁵, and R³⁶.

Some embodiments disclosed herein provide a compound of Formula IV having the proviso that a compound of Formula IV is not selected from the group consisting of:

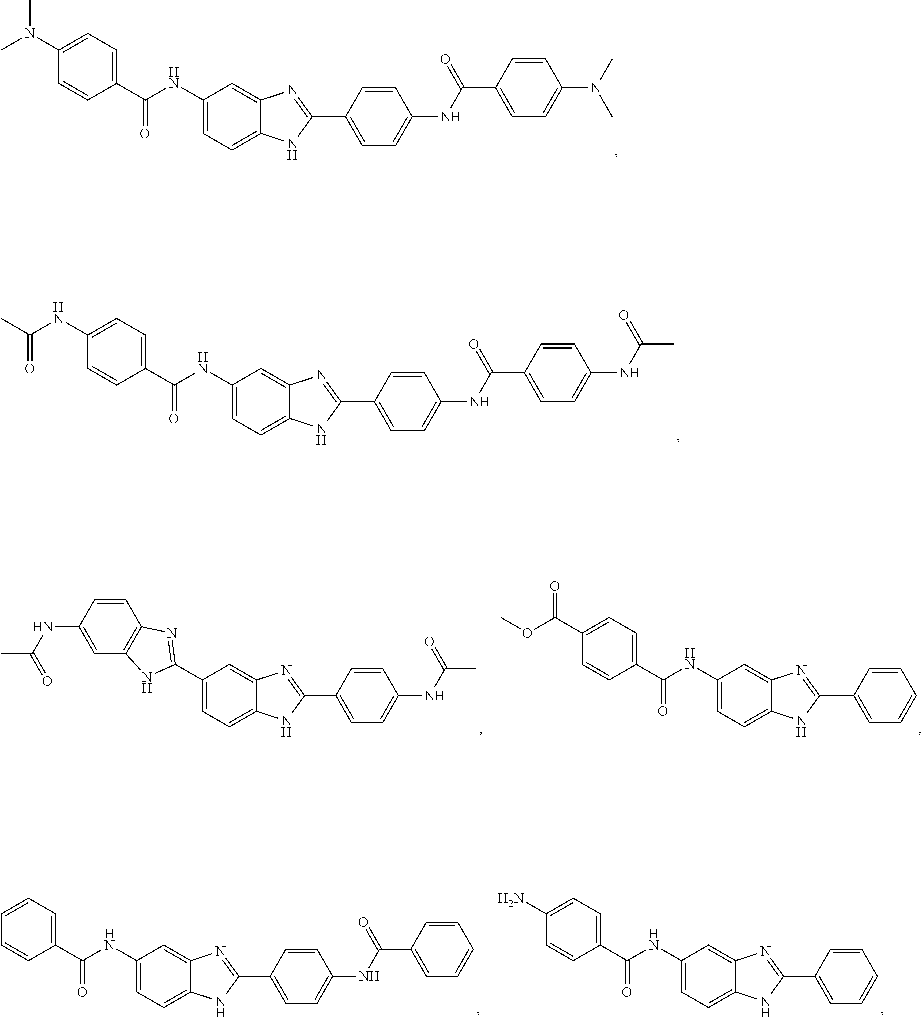

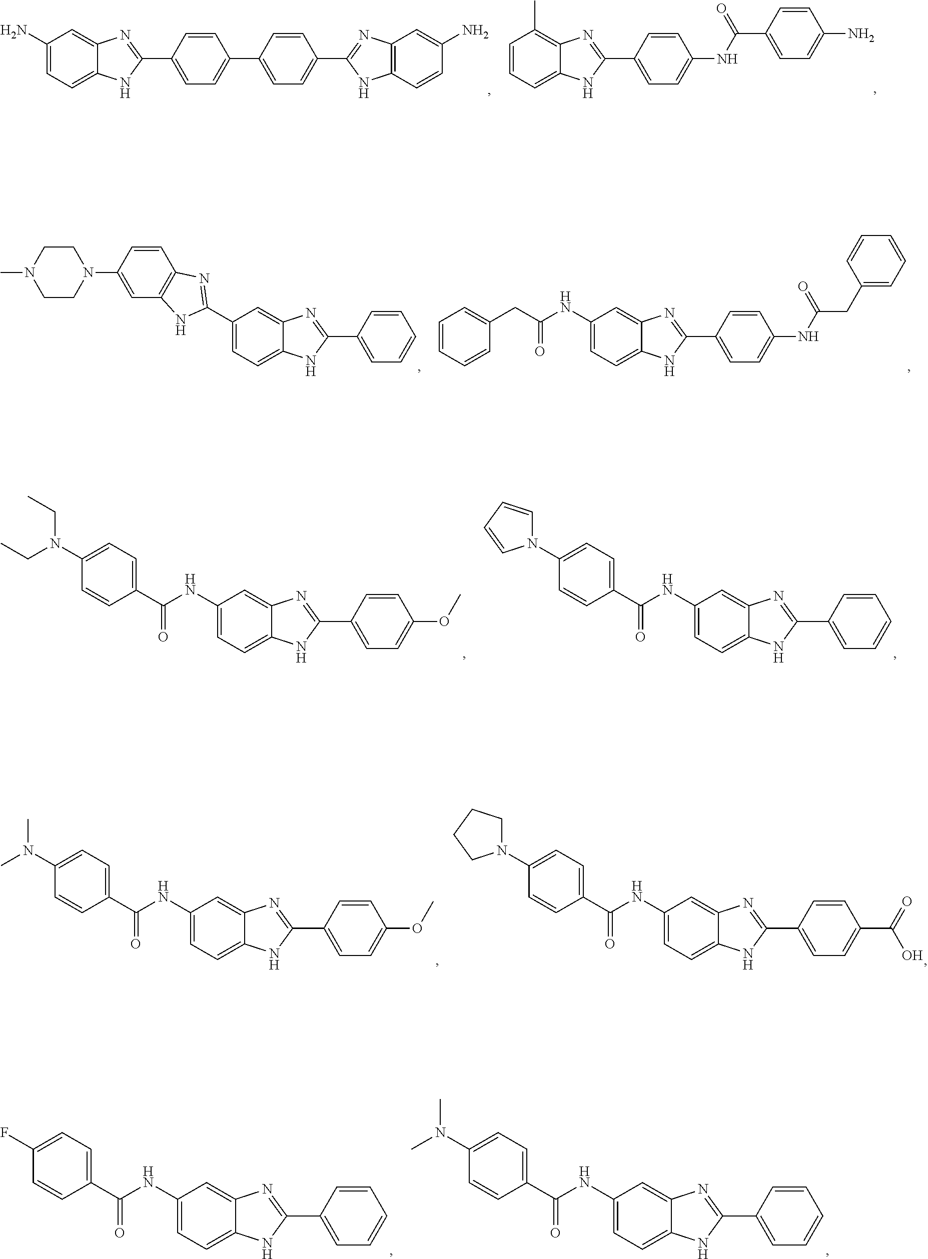

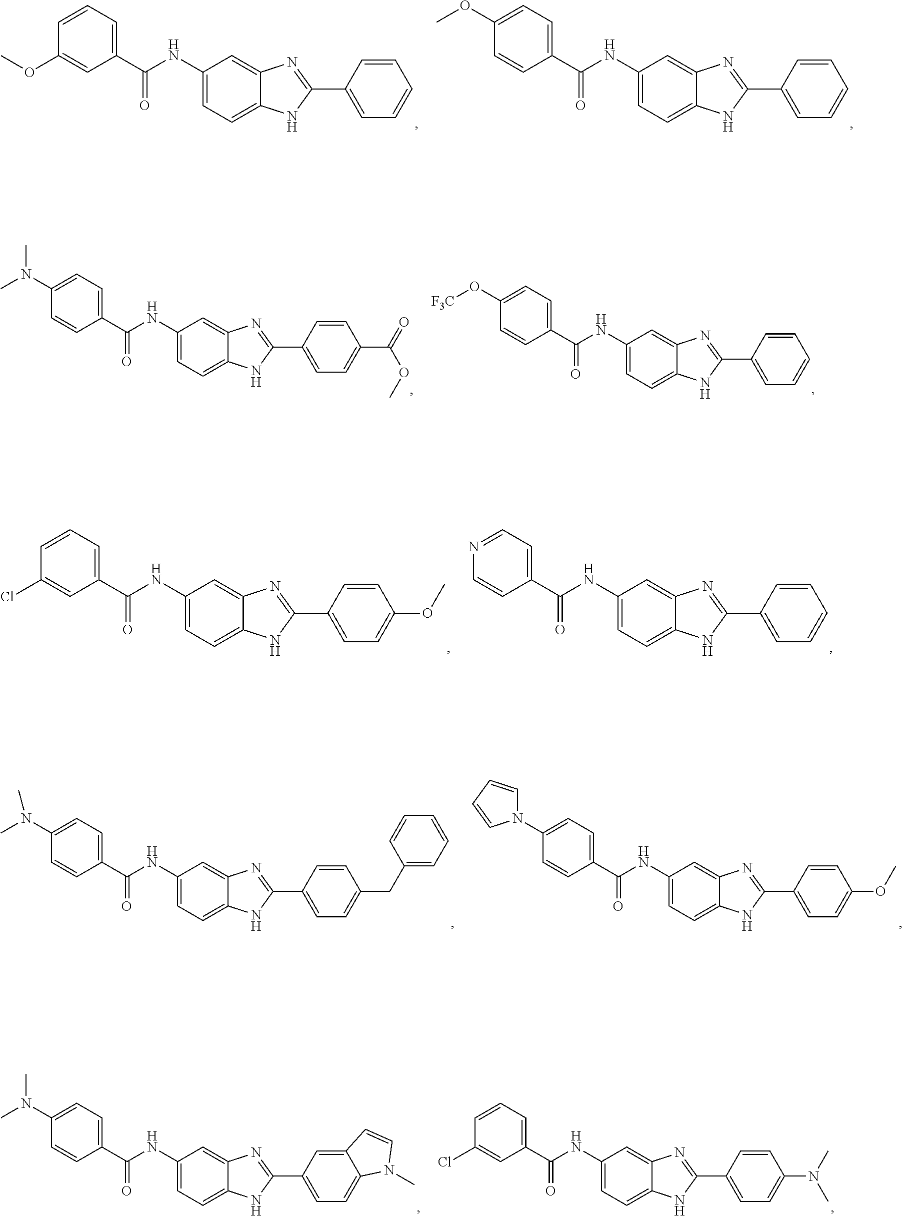

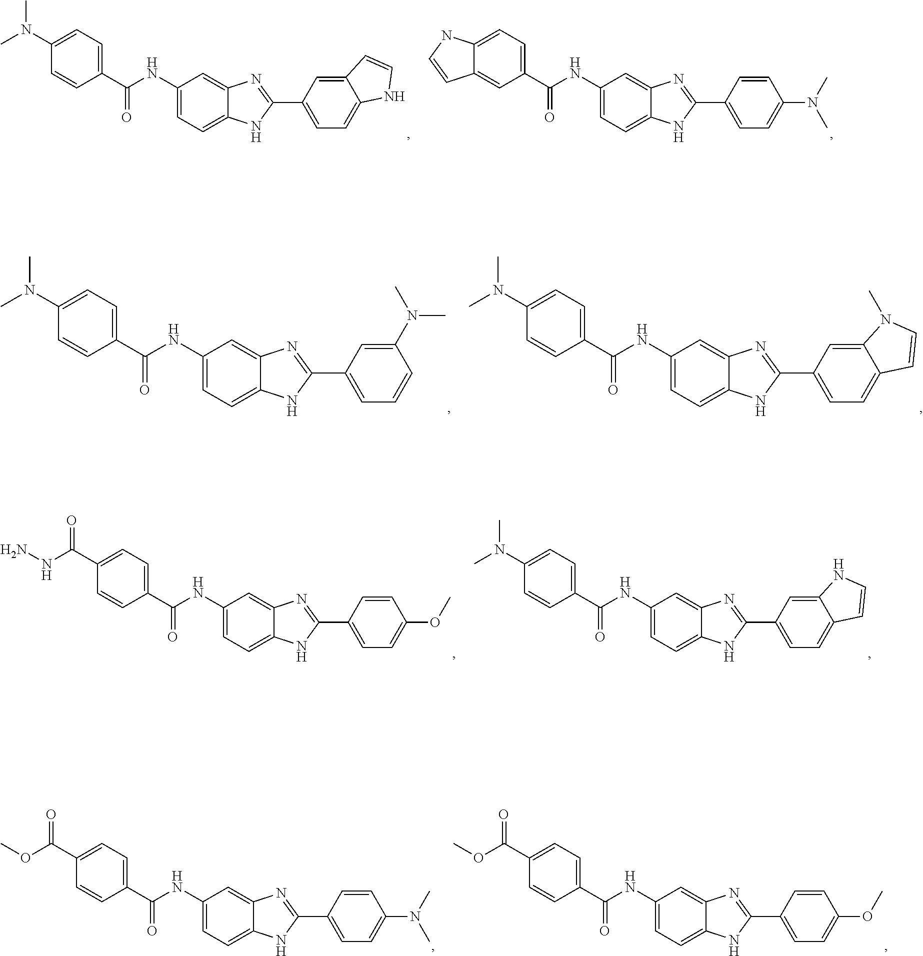

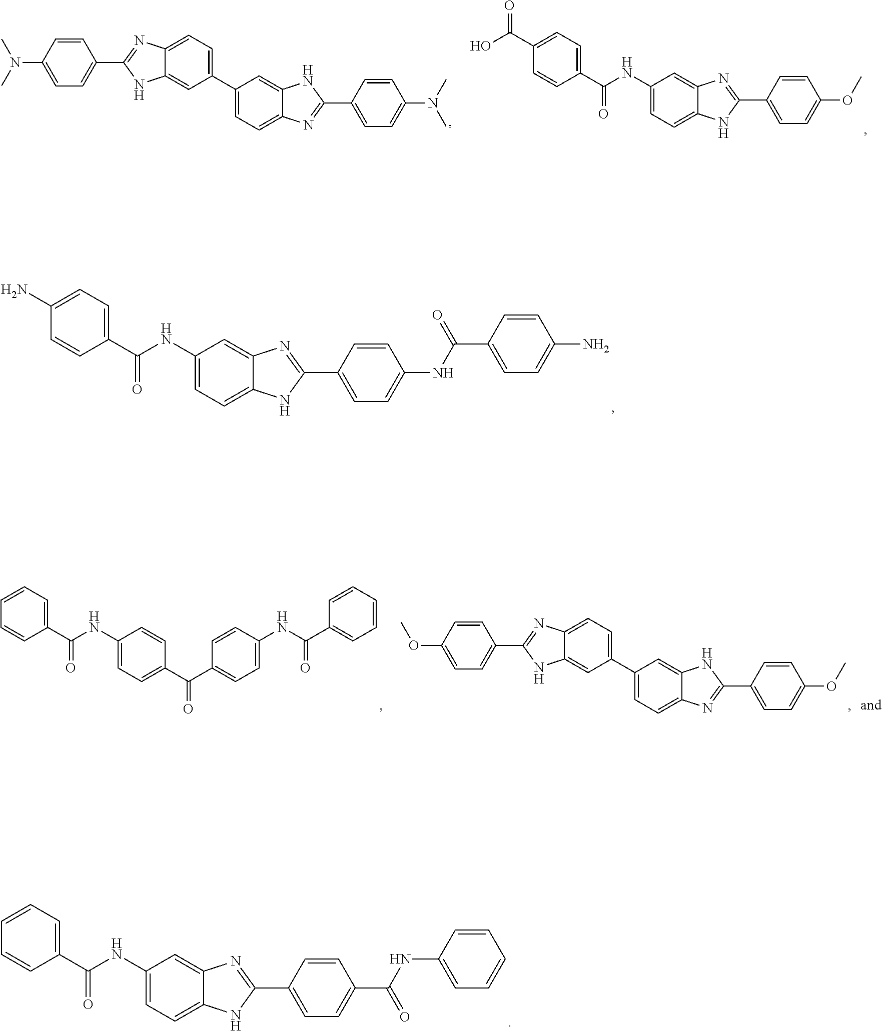

Some embodiments disclosed herein provide a compound of Formula V:

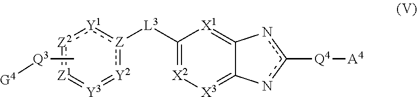

and pharmaceutically acceptable salts, esters, stereoisomers, tautomers or prodrugs thereof;

wherein:

G⁴ is selected from the group consisting of is selected from the group consisting of aryl and heteroaryl, each optionally substituted with one or more substituents selected from the group consisting of R⁴³ and R⁴⁴, said aryl and heteroaryl in the definition of G⁴ are each further optionally fused with an optionally substituted nonaromatic heterocycle or an optionally substituted nonaromatic carbocycle;

Q³ is selected from the group consisting of an ester, an amide, a urea, a carbamide, a thioamide, a sulfonamide; or Q³ is selected from the group consisting of —C(═O)O—, —C(═O)NR⁴⁵—, —C(═O)NHN═CH—, —NR⁴⁵C(═O)NR⁴⁵—, —NR⁴⁵C(═O)(CH₂)ₘO—, —OC(═O)NR⁴⁵—, —C(═S)NR⁴⁵—, —NR⁴⁵S(O)₁₋₂—, C₁-C₆ alkylideneamino, and

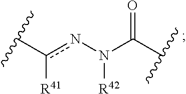

or when Y³ is C (carbon) substituted with -Q³-G⁴ then Q³ is optionally fused with Z¹ to form a five-member ring heterocycle;

L³ is selected from the group consisting of —C(═O)NR⁴⁵—, —O—C₁-C₈₋alkyl, —C(═NR⁴⁵)—, —NR⁴⁵C(═O)—(CH₂)ₘC(═O)NR⁴⁵—, and —NR⁴⁵C(═O)—(CH₂)ₘNR⁴⁵C(═O)—;

Q⁴ is selected from the group consisting of NR⁴⁸, and O (oxygen); or Q⁴ is null;

A⁴ is selected from the group consisting of C₁-C₆ alkyl, C₂-C₆ alkenyl, C₂-C₆ alkynyl, C₃-C₇ cycloalkyl, C₃-C₇ cycloalkenyl, a C₁-C₆ heteroalkyl, phenyl, pyridinyl, imidazolyl, and thienyl, each optionally substituted with one or more substituents selected from the group consisting of R⁴¹ and R⁴²;

X¹, X², and X³ are each independently selected from N (nitrogen) and CR⁴⁶

Y¹, Y², and Y³ are each independently selected from N (nitrogen) and CR⁴⁷;

Z, Z¹, and Z² are each independently selected from C (carbon), CH, and N (nitrogen);

R⁴¹ is independently selected from the group consisting of halogen, cyano, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl;

each R⁴² is independently selected from the group consisting of —(CH₂)ₘOR^{A}, —O(CH₂)ₘOR^{A}, —NR^{B}R^{C}, —C(═O)NR^{B}R^{C}, —C(═NNR^{B}R^{C})H, —(CH₂)ₘSR^{A}, —(CH₂)ₘR^{K}, —O(CH₂)ₘR^{K};

or R⁴¹ and R⁴² are linked to form an optionally substituted ring;

each R⁴³ is independently selected from the group consisting of halogen, cyano, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl;

each R⁴⁴ is independently selected from the group consisting of —(CH₂)ₘOR^{A}, —O(CH₂)ₘOR^{A}, —NR^{B}R^{C}, —C(═O)NR^{B}R^{C}, —C(═NNR^{B}R^{C})H, —(CH₂)ₘSR^{A}, —(CH₂)ₘR^{K}, —O(CH₂)ₘR^{K};

each R⁴⁵ is independently selected from the group consisting of hydrogen, an optionally substituted C₁-C₄ alkyl, and an optionally substituted C₁-C₆ heteroalkyl;

each R⁴⁶ and R⁴⁷ is independently selected from the group consisting of hydrogen, halogen, an optionally substituted C₁-C₆ alkyl, and an optionally substituted C₁-C₆ heteroalkyl;

R⁴⁸ is selected from the group consisting of hydrogen, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₃-C₇ cycloalkenyl, and an optionally substituted C₁-C₆ heteroalkyl;

each R^{A} is independently selected from the group consisting of hydrogen, C₁-C₆ alkyl, C₁-C₆ haloalkyl, C₁-C₆ heteroalkyl, and C₁-C₆ heterohaloalkyl;

each —NR^{B}R^{C} is separately selected, wherein R^{B} and R^{C} are each independently selected from the group consisting of hydrogen, —SO₂R^{F}, —C(═O)R^{F}, —(CH₂)ₘR^{F}, —SO₂NR^{D}R^{E}, —C(═O)NR^{D}R^{E}, —(CH₂)ₘNR^{D}R^{E}, C₁-C₆ alkyl, C₃-C₇ cycloalkyl, C₃-C₇ cycloalkenyl, C₁-C₆ haloalkyl, C₁-C₆ heteroalkyl, and C₁-C₆ heterohaloalkyl where the alkyl and the heteroalkyl are optionally fused with an aryl or heteroaryl; or —NR^{B}R^{C} is an optionally substituted non-aromatic heterocycle linked through a ring nitrogen atom; or —NR^{B}R^{C} is an optionally substituted C₁-C₆ alkylideneamino;

each —NR^{D}R^{E} is separately selected, wherein R^{D} and R^{E} are each independently selected from the group consisting of hydrogen, an optionally substituted C₁-C₆ alkyl, an optionally substituted C₃-C₇ cycloalkyl, an optionally substituted C₃-C₇ cycloalkenyl, an optionally substituted C₁-C₆ haloalkyl, an optionally substituted C₁-C₆ heteroalkyl, and —(CH₂)ₘR^{G}; or —NR^{D}R^{E} is an optionally substituted C₁-C₆ alkylideneaminyl; or —NR^{D}R^{E} is an optionally substituted non-aromatic heterocycle linked through a ring nitrogen atom;

each R^{F} is independently selected from the group consisting of hydrogen, an optionally substituted C₁-C₄ alkyl, an optionally substituted C₁-C₄ haloalkyl, an optionally substituted C₃-C₇ cycloalkyl, an optionally substituted C₃-C₇ cycloalkenyl, aryl and heteroaryl, where the aryl and heteroaryl in the definition of R^{F} are each optionally substituted with —C(═O)NR^{D}R^{E} or —NR^{D}R^{E};

each R^{G} is independently selected from the group consisting of an optionally substituted aryl and an optionally substituted heteroaryl;

each R^{K} is independently selected from the group consisting of —C(═O)NR^{D}R^{E}, —NR^{D}R^{E}, an optionally substituted aryl and an optionally substituted heteroaryl;

each m is independently 0, 1, or 2; and

each dashed line represents an optional double bond.

Some embodiments disclosed herein provide a compound of Formula V having the structure of Formula Va:

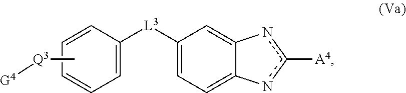

and pharmaceutically acceptable salts, esters, or prodrugs thereof, wherein G⁴ can be selected from the group consisting of hydrogen, halogen, —(CH₂)ₘOR^{A}, —O(CH₂)ₘOR^{A}, —NR^{B}R^{C}, an optionally substituted C₁-C₆ alkyl, an optionally substituted phenyl, an optionally substituted pyridinyl, an optionally substituted tetrazolyl, and an optionally substituted imidazolyl; Q³ can be selected from the group consisting of —C(═O)O—, —C(═O)NH—, —C(═O)NHNH—, —NR⁴⁵C(═O)NR⁴⁵—, —OC(═O)NR⁴⁵—, —C(═S)NR⁴⁵—, —NR⁴⁵S(O)₁₋₂—, C₁-C₆ alkylideneaminyl, and

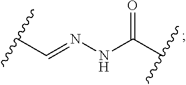 [SMILES: */C=N/NC(*)=O]

or Q³ can be null; A⁴ can be selected from the group consisting of a aryl and heteroaryl, each substituted with one or more substituents selected from the group consisting of R⁴¹ and R⁴², said aryl and heteroaryl in the definition of A⁴ can each be further optionally fused with an optionally substituted nonaromatic heterocycle or an optionally substituted nonaromatic carbocycle; L³ can be selected from —O— (oxygen), —S(O)₀₋₂—, —NR⁴⁵S(O)₀₋₂—, —NR⁴⁵C(═O)—(CH₂)ₘC(═O)NR⁴⁵—, —NR⁴⁵C(═O)—(CH₂)ₘNR⁴⁵C(═O)—, —NR⁴⁵—, —C(═O)—, —C(═S)—, —C(═O)NR⁴⁵—, —C(═NR⁴⁵)—, an optionally substituted C₁-C₈ alkyl, an optionally substituted C₁-C₈ heteroalkyl, an optionally substituted heterocycle, an optionally substituted aryl, and an optionally substituted heteroaryl; or L³ can be null; each R⁴¹ can be independently selected from the group consisting of hydrogen, halogen, and an optionally substituted C₁-C₃ alkyl; each R⁴² can be independently selected from the group consisting of hydrogen and an optionally substituted C₁-C₃ alkyl; each R⁴³ can be independently selected from the group consisting of halogen, an optionally substituted C₁-C₆ alkyl, an optionally substituted C₁-C₆ alkoxy, an optionally substituted C₃-C₇ cycloalkyl, an optionally substituted C₁-C₆ haloalkyl, an optionally substituted C₁-C₆ heteroalkyl; each R⁴⁴ can be independently selected from the group consisting of halogen, —(CH₂)ₘOR^{A}, —O(CH₂)ₘOR^{A}, —NR^{B}R^{C}, —C(═O)NR^{B}R^{C}, —C(═NNR^{B}R^{C})H, —(CH₂)ₘR^{K}, —O(CH₂)ₘR^{K}, an optionally substituted C₁-C₆ alkyl, an optionally substituted C₁-C₆ alkoxy, an optionally substituted C₃-C₇ cycloalkyl, an optionally substituted C₃-C₇ cycloalkenyl, an optionally substituted C₁-C₆ haloalkyl, and an optionally substituted C₁-C₆ heteroalkyl; each R⁴⁵ can be independently selected from the group consisting of hydrogen, and an optionally substituted C₁-C₄ alkyl; each R^{A} can be independently selected from the group consisting of hydrogen, C₁-C₆ alkyl, and C₁-C₆ haloalkyl; each —NR^{B}R^{C} can be separately selected, wherein R^{B} and R^{C} can each be independently selected from the group consisting of hydrogen, —SO₂R^{F}, —C(═O)R^{F}, —(CH₂)ₘR^{F}, —(CH₂)ₘOR^{F}, —SO₂NR^{D}R^{E}, —C(═O)NR^{D}R^{E}, —C(═NH)NR^{D}R^{E}, —(CH₂)ₘNR^{D}R^{E}, C₁-C₆ alkyl, C₃-C₇ cycloalkyl, C₃-C₇ cycloalkenyl, C₁-C₆ haloalkyl, C₁-C₆ heteroalkyl, and C₁-C₆ heterohaloalkyl where the alkyl and the heteroalkyl are optionally fused with an aryl or heteroaryl; or —NR^{B}R^{C} can be an optionally substituted non-aromatic heterocycle linked through a ring nitrogen atom; or —NR^{B}R^{C} can be an optionally substituted C₁-C₆ alkylideneamino; each —NR^{D}R^{E} can be separately selected, wherein R^{D} and R^{E} can each be independently selected from the group consisting of hydrogen, an optionally substituted C₁-C₆ alkyl, an optionally substituted C₃-C₇ cycloalkyl, an optionally substituted C₃-C₇ cycloalkenyl, an optionally substituted C₁-C₆ haloalkyl, an optionally substituted C₁-C₆ heteroalkyl, and —(CH₂)ₘR^{G}; or —NR^{D}R^{E} can be an optionally substituted C₁-C₆ alkylideneamino; or —NR^{D}R^{E} can be an optionally substituted non-aromatic heterocycle linked through a ring nitrogen atom; each R^{F} can be independently selected from the group consisting of hydrogen, an optionally substituted C₁-C₃ alkyl, an optionally substituted C₁-C₃ haloalkyl, an optionally substituted C₃-C₇ cycloalkyl, an optionally substituted C₃-C₇ cycloalkenyl, aryl and heteroaryl, where the aryl and heteroaryl in the definition of R^{F} can each be optionally substituted with —C(═O)NR^{D}R^{E} or —NR^{D}R^{E}; each R^{G} can be independently selected from an optionally substituted aryl and an optionally substituted heteroaryl; each R^{K} can be independently selected from the group consisting of —C(═O)NR^{D}R^{E}, —NR^{D}R^{E}, an optionally substituted aryl and an optionally substituted heteroaryl; each m can be independently 0, 1, or 2; and each dashed line represents an optionally double bond.

Some embodiments disclosed herein provide a compound of Formula Va, wherein G⁴ can be selected from the group consisting of hydrogen, halogen, —(CH₂)ₘOR^{A}, —O(CH₂)ₘOR^{A}, —NR^{B}R^{C}, an optionally substituted C₁-C₆ alkyl, an optionally substituted phenyl, an optionally substituted pyridinyl, an optionally substituted tetrazolyl, and an optionally substituted imidazolyl; Q³ can be selected from the group consisting of —C(═O)NH—, and

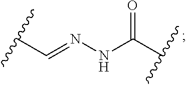 [SMILES: */C=N/NC(*)=O]

or Q³ can be null: A⁴ can be selected from the group consisting of phenyl, naphthyl, dihydrobenzofuranyl, 1,4-benzodioxanyl, benzotriazolyl, benzimidazolyl, benzofuranyl, and 2,1,3-benzoxadiazolyl, each optionally substituted with one or more substituents selected from the group consisting of, each optionally substituted with one or more substituents selected from the group consisting of R⁴¹ and R⁴²; L³ can be a 1-6 atom long linker comprising one or more groups selected from—NR⁴⁵—, —C(═O)—, —C(═S)—, and —C(═O)NR⁴⁵—; or L³ can be null; each R⁴¹ can be independently selected from the group consisting of halogen, an optionally substituted C₁-C₆ alkyl, and an optionally substituted C₁-C₆ alkoxy; each R⁴² can be independently selected from the group consisting of —(CH₂)ₘOR^{A}, —O(CH₂)ₘOR^{A}, —NR^{B}R^{C}, —C(═O)NR^{B}R^{C}, —C(═NNR^{B}R^{C})H, —(CH₂)ₘR^{K} and —O(CH₂)ₘR^{K}; each R⁴⁵ can be independently selected from the group consisting of hydrogen, and methyl; each R^{A} can be independently selected from the group consisting of hydrogen, C₁-C₄ alkyl, and C₁-C₄ haloalkyl; each —NR^{B}R^{C} can be separately selected, wherein R^{B} and R^{C} can each be independently selected from the group consisting of hydrogen, —C(═O)R^{F}, —(CH₂)ₘR^{F}, —SO₂NR^{D}R^{E}, —C(═O)NR^{D}R^{E}, —C(═NH)NR^{D}R^{E}, C₁-C₆ alkyl, C₃-C₇ cycloalkyl, C₃-C₇ cycloalkenyl, and C₁-C₆ haloalkyl; or —NR^{B}R^{C} can be an optionally substituted non-aromatic heterocycle linked through a ring nitrogen atom; or —NR^{B}R^{C} can be an optionally substituted C₁-C₆ alkylideneamino; each —NR^{D}R^{E} can be separately selected, wherein R^{D} and R^{E} can each be independently selected from the group consisting of hydrogen, an optionally substituted C₁-C₆ alkyl, an optionally substituted C₃-C₇ cycloalkyl, an optionally substituted C₃-C₇ cycloalkenyl, an optionally substituted C₁-C₆ haloalkyl, and —(CH₂)ₘR^{G}; or —NR^{D}R^{E} can be an optionally substituted C₁-C₆ alkylideneaminyl; or —NR^{D}R^{E} can be an optionally substituted non-aromatic heterocycle linked through a ring nitrogen atom; each R^{F} can be independently selected from the group consisting of hydrogen, an optionally substituted C₁-C₃ alkyl, an optionally substituted C₃-C₆ cycloalkyl, an optionally substituted C₃-C₆ cycloalkenyl, an optionally substituted C₁-C₃ haloalkyl, aryl and heteroaryl, where the aryl and heteroaryl in the definition of R^{F} can each be optionally substituted with —C(═O)NR^{D}R^{E} or —NR^{D}R^{E}; each R^{G} can be independently selected from an optionally substituted aryl and an optionally substituted heteroaryl; each m can be independently 0, 1, or 2; and each dashed line represents an optional double bond.

Some embodiments disclosed herein provide a compound of Formula V, wherein G⁴ can be selected from the group consisting of hydrogen, halogen, —(CH₂)ₘOR^{A}, —O(CH₂)ₘOR^{A}, —NR^{B}R^{C}, an optionally substituted C₁-C₆ alkyl, an optionally substituted phenyl, an optionally substituted pyridinyl, an optionally substituted tetrazolyl, and an optionally substituted imidazolyl; Q³ can be selected from the group consisting of an ester, an amide, a urea, a carbamide, a thioamide, an imidamide, a sulfonamide, and a hydrazide derivative; or Q³ is selected from the group consisting of —C(═O)O—, —C(═O)NH—, —NR⁴⁵C(═O)NR⁴⁵—, —OC(═O)NR⁴⁵—, —C(═S)NR⁴⁵—, —NR⁴⁵S(O)₁₋₂—, C₁-C₆ alkylideneaminyl, and

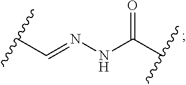 [SMILES: */C=N/NC(*)=O]

or Q³ can be null; A⁴ can be selected from the group consisting of a aryl and heteroaryl, each substituted with one or more substituents selected from the group consisting of R⁴¹ and R⁴², said aryl and heteroaryl in the definition of A⁴ can each be further optionally fused with an optionally substituted nonaromatic heterocycle or an optionally substituted nonaromatic carbocycle; L³ can be selected from —O— (oxygen), —S(O)₁₋₂—, —NR⁴⁵S(O)₁₋₂—, —NR⁴⁵—, —C(═O)—, —C(═S)—, —C(═O)NR⁴⁵—, —C(═NR⁴⁵)—, —NR⁴⁵C(═O)—(CH₂)ₘC(═O)NR⁴⁵—, —NR⁴⁵C(═O)—(CH₂)ₘNR⁴C(═O)—, an optionally substituted C₁-C₈ alkyl, an optionally substituted C₁-C₈ heteroalkyl, an optionally substituted heterocycle, an optionally substituted aryl, and an optionally substituted heteroaryl; or L³ can be null; each R⁴¹ can be independently selected from the group consisting of halogen, cyano, an optionally substituted C₁-C₆ alkyl, an optionally substituted C₁-C₆ alkoxy, an optionally substituted C₁-C₆ haloalkyl, an optionally substituted C₁-C₆ heteroalkyl, and null; each R⁴² can be independently selected from the group consisting of —(CH₂)ₘOR^{A}, —NR^{B}R^{C}, —C(═O)NR^{B}R^{C}, —C(═NNR^{B}R^{C})H, —(CH₂)ₘR^{K}, —O(CH₂)ₘR^{K}, an optionally substituted C₁-C₆ alkyl, an optionally substituted C₁-C₆ alkoxy, an optionally substituted C₁-C₆ haloalkyl, and an optionally substituted C₁-C₆ heteroalkyl; each R⁴⁵ can be independently selected from the group consisting of hydrogen, and an optionally substituted C₁-C₄ alkyl; each R^{A} can be independently selected from the group consisting of hydrogen, C₁-C₆ alkyl, and C₁-C₆ haloalkyl; each —NR^{B}R^{C} can be separately selected, wherein R^{B} and R^{C} can each be independently selected from the group consisting of hydrogen, —SO₂R^{F}, —C(═O)R^{F}, —(CH₂)ₘR^{F}, —(CH₂)ₘOR^{F}, —SO₂NR^{D}R^{E}, —C(═O)NR^{D}R^{E}, —C(═NH)NR^{D}R^{E}, C₁-C₆ alkyl, C₁-C₆ haloalkyl, C₁-C₆ heteroalkyl, and C₁-C₆ heterohaloalkyl where the alkyl and the heteroalkyl are optionally fused with an aryl or heteroaryl; or —NR^{B}R^{C} can be an optionally substituted non-aromatic heterocycle linked through a ring nitrogen atom; or —NR^{B}R^{C} can be an optionally substituted C₁-C₆ alkylideneamino; each —NR^{D}R^{E} can be separately selected, wherein R^{D} and R^{E} can each be independently selected from the group consisting of hydrogen, an optionally substituted C₁-C₆ alkyl, an optionally substituted C₁-C₆ haloalkyl, an optionally substituted C₁-C₆ heteroalkyl, and —(CH₂)ₘR^{G}; or —NR^{D}R^{E} can be an optionally substituted C₁-C₆ alkylideneaminyl; or —NR^{D}R^{E} can be an optionally substituted non-aromatic heterocycle linked through a ring nitrogen atom; each R^{F} can be independently selected from the group consisting of hydrogen, C₁-C₃ alkyl, C₁-C₃ haloalkyl, aryl and heteroaryl, where the aryl and heteroaryl in the definition of R^{F} can each be optionally substituted with —C(═O)NR^{D}R^{E} or —NR^{D}R^{E}; each R^{G} can be independently selected from an optionally substituted aryl and an optionally substituted heteroaryl; each m can be independently 0, 1, or 2; and one dashed line represents a double bond.

Some embodiments disclosed herein provide a compound of Formula Va having the structure of Formula Vb:

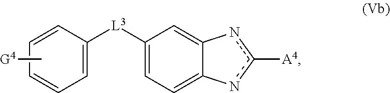

and pharmaceutically acceptable salts, wherein G⁴ can be selected from the group consisting of hydrogen, halogen, fluoro, chloro, bromo, —OR^{A}, —O(CH₂)ₘOR^{A}, —NR^{B}R^{C}, an optionally substituted C₁-C₆ alkyl, an optionally substituted phenyl, an optionally substituted pyridinyl, an optionally substituted tetrazolyl, and an optionally substituted imidazolyl; A⁴ can be phenyl optionally substituted with one or more substituents selected from the group consisting of R⁴¹ and R⁴², where the phenyl in the definition of A⁴ can be further optionally fused with an optionally substituted nonaromatic heterocycle or an optionally substituted nonaromatic carbocycle; L³ can be selected from —C(═O)NR⁴⁵—, —NR⁴⁵C(═O)—(CH₂)ₘC(═O)NR⁴⁵—, —NR⁴⁵C(═O)—(CH₂)ₘNR⁴⁵C(═O)—, and an optionally substituted heteroaryl; or L³ can be null; each R⁴¹ can be independently selected from the group consisting of halogen, an optionally substituted C₁-C₆ alkyl, an optionally substituted C₁-C₆ alkoxy; each R⁴² can be independently selected from the group consisting of —(CH₂)ₘOR^{A}, —O(CH₂)ₘOR^{A}, —NR^{B}R^{C}, —C(═O)NR^{B}R^{C}, —C(═NNR^{B}R^{C})H, —(CH₂)ₘR^{K}, —O(CH₂)ₘR^{K}; each R⁴s can be independently selected from the group consisting of hydrogen, and an optionally substituted C₁-C₄ alkyl; each R^{A} is independently selected from the group consisting of hydrogen, C₁-C₆ alkyl, and C₁-C₆ haloalkyl; each —NR^{B}R^{C} can be separately selected, wherein R^{B} and R^{C} can each be independently selected from the group consisting of hydrogen, —SO₂R^{F}, —C(═O)R^{F}, —(CH₂)ₘR^{F}, —(CH₂)ₘOR^{F}, —SO₂NR^{D}R^{E}, —C(═O)NR^{D}R^{E}, —C(═NH)NR^{D}R^{E}, —(CH₂)ₘNR^{D}R^{E}, C₁-C₆ alkyl, C₃-C₇ cycloalkyl, C₃-C₇ cycloalkenyl, C₁-C₆ haloalkyl, C₁-C₆ heteroalkyl, and C₁-C₆ heterohaloalkyl where the alkyl and the heteroalkyl are optionally fused with an aryl or heteroaryl; or —NR^{B}R^{C} can be an optionally substituted non-aromatic heterocycle linked through a ring nitrogen atom; each —NR^{D}R^{E} can be separately selected, wherein R^{D} and R^{E} can each be independently selected from the group consisting of hydrogen, an optionally substituted C₁-C₆ alkyl, an optionally substituted C₃-C₇ cycloalkyl, an optionally substituted C₃-C₇ cycloalkenyl, an optionally substituted C₁-C₆ haloalkyl, an optionally substituted C₁-C₆ heteroalkyl, and —(CH₂)ₘR^{G}; or —NR^{D}R^{E} can be an optionally substituted C₁-C₆ alkylideneaminyl; or —NR^{D}R^{E} can be an optionally substituted non-aromatic heterocycle linked through a ring nitrogen atom; each R^{F} can be independently selected from the group consisting of hydrogen, an optionally substituted C₁-C₃ alkyl, an optionally substituted C₃-C₇ cycloalkyl, aryl and heteroaryl, where the aryl and heteroaryl in the definition of R^{F} can each be optionally substituted with —C(═O)NR^{D}R^{E} or —NR^{D}R^{E}; each R^{G} can be independently selected from an optionally substituted aryl and an optionally substituted heteroaryl; each R^{K} can be independently selected from the group consisting of —C(═O)NR^{D}R^{E}, —NR^{D}R^{E}, an optionally substituted aryl and an optionally substituted heteroaryl; each m can be independently 0, 1, or 2; and, each dashed line represents an optionally double bond.

Some embodiments disclosed herein provide a compound of Formula Vb, wherein G⁴ can be selected from the group consisting of hydrogen, fluoro, chloro, bromo, imidazolyl, tetrazolyl, N-methyl-N-(2-hydroxyethyl)aminyl, methylaminosulfonamido, 2-hydroxyethyloxy, —(CH₂)ₘOR^{A}, —O(CH₂)ₘOR^{A}, and —NR^{B}R^{C}; A⁴ can be phenyl optionally substituted with one or more substituents selected from the group consisting of R⁴¹ and R⁴², where the phenyl in the definition of G⁴ can be further optionally fused with an optionally substituted nonaromatic heterocycle or an optionally substituted nonaromatic carbocycle; L³ can be selected from the group consisting of —C(═O)NH—, —NHC(═O)—C(═O)NH— and an optionally substituted heteroaryl; or L³ can be null; each R⁴¹ can be an optionally substituted C₁-C₆ alkoxy; each R⁴² can be independently selected from the group consisting of —(CH₂)ₘOR^{A}, —O(CH₂)ₘOR^{A}, —NR^{B}R^{C}, —C(═O)NR^{B}R^{C}, —C(═NNR^{B}R^{C})H, —(CH₂)ₘR^{K}, and —O(CH₂)ₘR^{K}; each R^{A} can be independently selected from the group consisting of hydrogen, and C₁-C₆ alkyl; each —NR^{B}R^{C} can be separately selected, wherein R^{B} and R^{C} can each be independently selected from the group consisting of hydrogen, —SO₂R^{F}, —C(═O)R^{F}, —(CH₂)ₘR^{F}, —OR^{F}, —SO₂NR^{D}R^{E}, —(CH₂)ₘNR^{D}R^{E}, C₁-C₆ alkyl, and C₃-C₇ cycloalkyl; or —NR^{B}R^{C} can be selected from the group consisting of pyrrolidinyl, morpholinyl, 4-methylpiperazinyl, piperazinyl, piperidinyl, 3-hydroxypyrrolidinyl, and 4-hydroxypiperidinyl, each optionally substituted with oxo; each —NR^{D}R^{E} can be separately selected, wherein R^{D} and R^{E} can each be independently selected from the group consisting of hydrogen, an optionally substituted C₁-C₆ alkyl, an optionally substituted C₃-C₆ cycloalkyl, and —(CH₂)ₘR^{G}; or —NR^{D}R^{E} can be selected from the group consisting of pyrrolidinyl, morpholinyl, 4-methylpiperazinyl, piperazinyl, piperidinyl, 3-hydroxypyrrolidinyl, and 4-hydroxypiperidinyl, each optionally substituted with oxo; each R^{F} can be independently selected from the group consisting of hydrogen, an optionally substituted C₁-C₃ alkyl, an optionally substituted C₃-C₆ cycloalkyl, aryl and heteroaryl, where the aryl and heteroaryl in the definition of R^{F} can each be optionally substituted with —NR^{D}R^{E}; each R^{G} can be independently selected from an optionally substituted aryl and an optionally substituted heteroaryl; each R^{K} can be independently selected from the group consisting of an optionally substituted aryl and an optionally substituted heteroaryl; each m can be independently 0, 1, or 2; and each dashed line represents an optionally double bond.

Some embodiments disclosed herein provide a compound of Formula V, having the proviso that a compound of Formula V is not selected from the group consisting of:

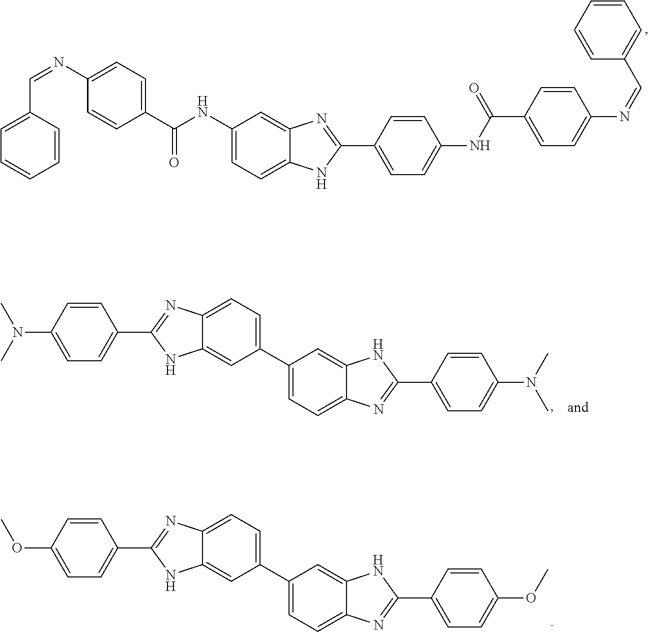

Some embodiments disclosed herein provide a compound of Formula VI:

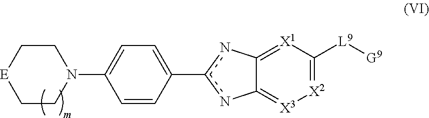

and pharmaceutically acceptable salts, esters, stereoisomers, tautomers or prodrugs thereof;

wherein:

E is selected from the group consisting of 0 (oxygen), S (sulfur), NR⁴¹ and CR⁴²R⁴³;

R⁴¹ is selected from the group consisting of hydrogen, halogen, cyano, —C(═O)R^{C}, C₁-C₆ haloalkyl, C₁-C₆ heteroalkyl, and an optionally substituted C₁-C₆ alkyl;

R⁴² and R⁴³ are each independently selected from the group consisting of hydrogen, halogen, —OR^{AA}, —OR^{C}c, —NR^{A}R^{B}, —NR^{C}R^{D}, —SR^{AA}, —(CH₂)ₘR^{E}, —CONR^{C}R^{D}, an optionally substituted C₁-C₆ alkyl, an optionally substituted C₁-C₆ haloalkyl, and an optionally substituted C₁-C₆ heteroalkyl; or CR⁴²R⁴³ is an optionally substituted C₃-C₇ cycloalkyl;

X¹, X², and X³ are each independently selected from the group consisting of N (nitrogen) and CR⁴¹;

G⁹ is selected from the group consisting of aryl and heteroaryl, each optionally substituted with one or more substituents selected from the group consisting of R⁴⁴ and R⁴⁵, said aryl and heteroaryl in the definition of G⁹ are each further optionally fused with an optionally substituted nonaromatic heterocycle or an optionally substituted nonaromatic carbocycle;

each R⁴⁴ is separately selected from the group consisting of halogen, cyano, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted C₁-C₆ haloalkyl, and an optionally substituted C₁-C₆ heteroalkyl;

each R⁴⁵ is separately selected from the group consisting of hydrogen, halogen, —OR^{AA}, —OR^{C}, —NR^{A}R^{B}, —NR^{C}R^{D}, —SR^{AA}, —(CH₂)ₘR^{E}, C₁-C₆ haloalkyl, C₁-C₆ heteroalkyl, and an optionally substituted C₁-C₆ alkyl;

each R^{AA} is independently selected from the group consisting of hydrogen, —(CH₂)ₘSO₂R^{F}, —(CH₂)ₘC(═O)R^{F}, —(CH₂)ₘC(═O)NR^{C}R^{D}, an optionally substituted C₁-C₈ alkyl, an optionally substituted C₁-C₈ alkoxy, an optionally substituted C₂-C₈ alkenyl, an optionally substituted C₂-C₈ alkynyl, and an optionally substituted C₃-C₇ cycloalkyl, where said C₃-C₇ cycloalkyl is optionally fused with an aryl or heteroaryl;

each R^{BB} is independently selected from the group consisting of hydrogen, an optionally substituted C₁-C₈ alkyl, an optionally substituted C₁-C₈ alkoxy, an optionally substituted C₂-C₈ alkenyl, an optionally substituted C₂-C₈ alkynyl, an optionally substituted C₃-C₇ cycloalkyl, and (CH₂)ₘR^{E};

each —NR^{A}R^{B} is separately selected, wherein R^{A} and R^{B} are each independently selected from the group consisting of hydrogen, —(CH₂)ₘSO₂R^{F}, —(CH₂)ₘC(═O)R^{F}, —(CH₂)ₘC(═O)NR^{C}R^{D}, an optionally substituted C₁-C₈ alkyl, an optionally substituted C₁-C₈ alkoxy, an optionally substituted C₂-C₈ alkenyl, an optionally substituted C₂-C₈ alkynyl, and an optionally substituted C₃-C₇ cycloalkyl, where said C₃-C₇ cycloalkyl is optionally fused with an aryl or heteroaryl; or —NR^{A}R^{B} is an optionally substituted non-aromatic heterocycle linked through a ring nitrogen atom optionally fused with an aryl or heteroaryl; or —NR^{A}R^{B} is an optionally substituted C₁-C₆ alkylideneamino;

each —NR^{C}R^{D} is separately selected, wherein R^{C} and R^{D} are each independently selected from the group consisting of hydrogen, an optionally substituted C₁-C₈ alkyl, an optionally substituted C₁-C₈ alkoxy, an optionally substituted C₂-C₈ alkenyl, an optionally substituted C₂-C₈ alkynyl, an optionally substituted C₃-C₇ cycloalkyl, and (CH₂)ₘR^{E}; or —NR^{C}R^{D} is an optionally substituted C₁-C₈ alkylideneamino; or —NR^{C}R^{D} is an optionally substituted non-aromatic heterocycle linked through a ring nitrogen atom;

each R^{E} is separately selected from the group consisting of an optionally substituted aryl and an optionally substituted heteroaryl;

each R^{F} is separately selected from the group consisting of hydrogen, a an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted aryl and an optionally substituted heteroaryl;

L⁹ is selected from the group consisting of —(CH₂)ₘC(═O)NR⁴⁶(CH₂)_{q}—, —(CH₂)ₘC(═O)NR⁴⁶(CH₂)_{q}C(═O)NR⁴⁶—, —S(O)₂NH—, O (oxygen), —NR⁴⁶—, —OC(═O)O—, —OC(═O)NH—, —NHC(═O)NH—, —NHC═SNH—, —C(═NR⁴⁶)—, —C(═O)NR⁴⁶—, —C(═S)NR⁴⁶—; or L⁹ is null;

each R⁴⁶ is independently selected from the group consisting of hydrogen, C₁-C₆ haloalkyl, and an optionally substituted C₁-C₆ alkyl;

each m is independently 0, 1, or 2;

each q is independently 1, 2, 3, 4, 5, or 6; and

any bond represented by a dashed and solid line represents a bond selected from the group consisting of a single bond and a double bond.

Some embodiments disclosed herein provide a compound of Formula VI having the structure of Formula VIa:

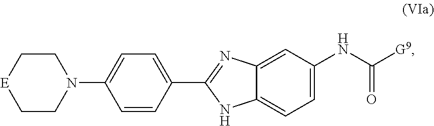

and pharmaceutically acceptable salts.

Some embodiments disclosed herein provide a compound of Formula VII:

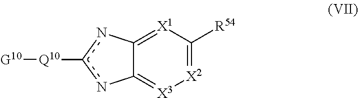

and pharmaceutically acceptable salts, esters, stereoisomers, tautomers or prodrugs thereof;

wherein:

G¹⁰ is selected from the group consisting of C₁-C₆ alkyl, C₁-C₆ alkoxy, C₂-C₆ alkenyl, C₂-C₆ alkynyl, C₃-C₇ cycloalkyl, C₃-C₇ cycloalkenyl, C₁-C₆ haloalkyl, C₁-C₆ heteroalkyl, heterocycle, aryl and heteroaryl, each optionally substituted with one or more substituents selected from the group consisting of R⁵¹, R⁵², and R⁵³, said aryl and heteroaryl in the definition of G¹⁰ are each further optionally fused with an optionally substituted nonaromatic heterocycle or an optionally substituted nonaromatic carbocycle;

Q¹⁰ is selected from the group consisting of Q¹¹, Q¹¹-Q¹², and Q¹¹-Q¹²-Q¹³;

Q¹¹ and Q¹³ are each independently selected from the group consisting of piperazinyl, —C(═O)O—, —C(═O)NR⁵¹—, —NR¹C(═O)NR⁵¹—, —OC(═O)NR⁵¹—, —C(═S)NR⁵¹—, —NR⁵¹S(O)₁₋₂—, —(CH₂)ₘC(═O)NR⁵¹(CH₂)_{q}—, and —(CH₂)ₘC(═O)NR(CH₂)_{q}C(═O)NR⁵¹—;

Q¹² is selected from the group consisting of an optionally substituted aryl, an optionally substituted heteroaryl and an optionally substituted heterocycle;

each R⁵¹ is separately selected from the group consisting of hydrogen, halogen, cyano, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted C₃-C₇ cycloalkenyl, an optionally substituted C₁-C₆ haloalkyl, and an optionally substituted C₁-C₆ heteroalkyl;

each R⁵² is separately selected from the group consisting of —(CH₂)ₘOR^{A}, —(CH₂)ₘNR^{B}R^{C}, —(CH₂)ₘSO₂NR^{B}R^{C}, and —(CH₂)ₘSR^{A};

each R⁵³ is separately selected from the group consisting of —(CH₂)ₘOR^{D}, —(CH₂)ₘNR^{E}R^{F}, —(CH₂)ₘS(O)₀₋₂R^{D}, —(CH₂)ₘNO₂, —(CH₂)ₘCN, and —(CH₂)ₘR^{G};

each R⁵⁴ is separately selected from the group consisting of hydrogen, —(CH₂)ₘOR^{A}, —(CH₂)ₘNR^{B}R^{C}, —O(CH₂)ₘNR^{B}R^{C}, —C(═O)NR^{B}R^{C}, —(CH₂)ₘSR^{A}, —(CH₂)ₘR^{G}, —O(CH₂)ₘR^{G}, —(CH₂)ₘSO₂NR^{B}R^{C}, —(CH₂)ₘCN, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted C₃-C₇ cycloalkenyl, an optionally substituted C₁-C₆ haloalkyl, and an optionally substituted C₁-C₆ heteroalkyl;

each R^{A} is separately selected from the group consisting of hydrogen, an optionally substituted C₁-C₆ alkyl, an optionally substituted C₂-C₆ alkenyl, an optionally substituted C₂-C₆ alkynyl, an optionally substituted C₃-C₇ cycloalkyl, an optionally substituted C₃-C₇ cycloalkenyl, an optionally substituted C₁-C₆ haloalkyl, and an optionally substituted C₁-C₆ heteroalkyl;

each —NR^{B}R^{C} is separately selected, wherein R^{B} and R^{C} are each independently selected from the group consisting of hydrogen, —(CH₂)ₘSO₂R^{H}, —(CH₂)ₘCOR^{H}, —(CH₂)ₘCONR^{E}R^{F}, an optionally substituted C₁-C₈ alkyl, an optionally substituted C₁-C₈ alkoxy, an optionally substituted C₂-C₈ alkenyl, an optionally substituted C₂-C₈ alkynyl, an optionally substituted C₃-C₇ cycloalkyl, and —(CH₂)ₘR^{G} where said C₃-C₇ cycloalkyl is optionally fused with an aryl or heteroaryl; or —NR^{B}R^{C} or is an optionally substituted non-aromatic heterocycle linked through a ring nitrogen atom optionally fused with an aryl or heteroaryl; or —NR^{B}R^{C} is an optionally substituted C₁-C₈ alkylideneamino;

each R^{D} is separately selected from the group consisting of hydrogen, an optionally substituted C₁-C₈ alkyl, an optionally substituted C₂-C₈ alkenyl, an optionally substituted C₂-C₈ alkynyl, an optionally substituted C₃-C₈ cycloalkyl, an optionally substituted C₃-C₈ cycloalkenyl, an optionally substituted C₁-C₈ haloalkyl, and an optionally substituted C₁-C₈ heteroalkyl;

each —NR^{E}R^{F} is separately selected, wherein R^{E} and R^{F} are each independently selected from the group consisting of hydrogen, an optionally substituted C₁-C₈ alkyl, an optionally substituted C₁-C₈ alkoxy, an optionally substituted C₂-C₈ alkenyl, an optionally substituted C₂-C₈ alkynyl, an optionally substituted C₃-C₇ cycloalkyl, and (CH₂)ₘR^{G}; or —NR^{E}R^{F} or is an optionally substituted non-aromatic heterocycle linked through a ring nitrogen atom; or —NR^{C}R^{D} is an optionally substituted C₁-C₈ alkylideneamino;

each R^{G} is separately selected from a substituted or unsubstituted aryl and a substituted or unsubstituted heteroaryl;

each R^{H} is separately selected from the group consisting of hydrogen, a C₁-C₆ alkyl, C₂-C₆ alkenyl, C₂-C₆ alkynyl, C₃-C₇ cycloalkyl, C₃-C₇ cycloalkenyl, C₁-C₆ haloalkyl, C₁-C₆ heteroalkyl, an optionally substituted heterocycle, an optionally substituted aryl, and an optionally substituted heteroaryl;

X¹, X², and X³ are each independently selected from the group consisting of N (nitrogen) and CR⁴⁷;

each R⁴⁷ is separately selected from the group consisting of hydrogen, halogen, an optionally substituted C₁-C₆ alkyl, and an optionally substituted C₁-C₆ heteroalkyl

each m is independently 0, 1, 2, or 3; and

any bond represented by a dashed and solid line represents a bond selected from the group consisting of a single bond and a double bond.

Some embodiments disclosed herein provide a compound of Formula VII having the structure of Formula VIIa:

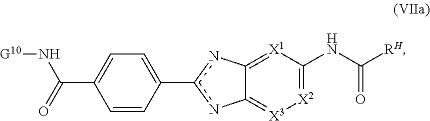

and pharmaceutically acceptable salts.

Some embodiments disclosed herein provide a compound of Formula VII, having the proviso that a compound of Formula VII is not selected from the group consisting of:

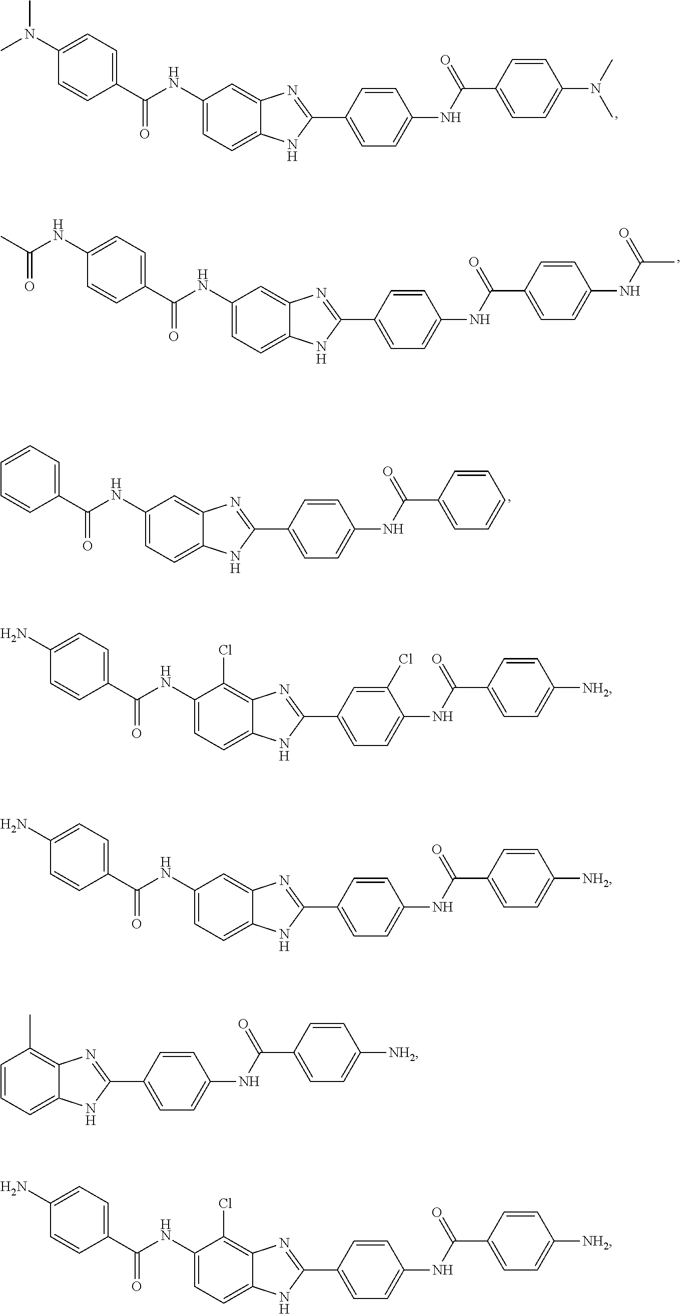

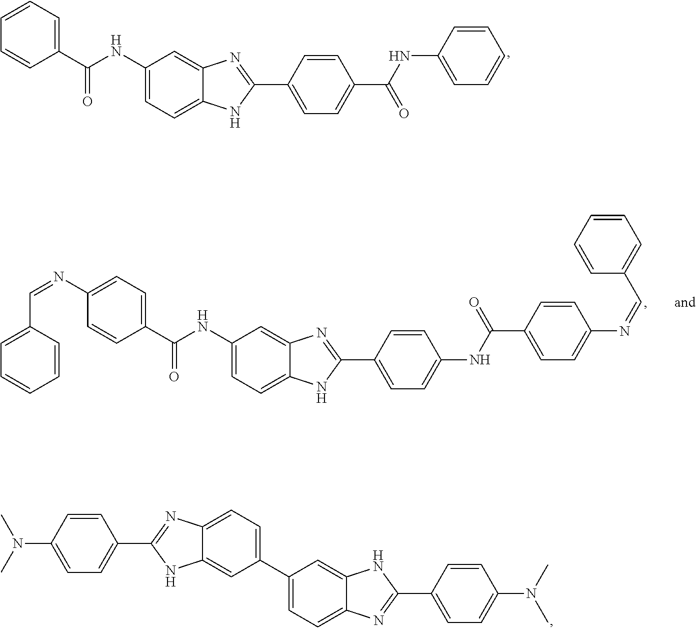

Some embodiments disclosed herein provide a compound of Formula VIII:

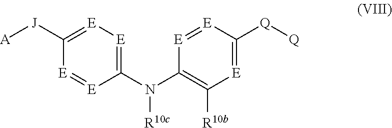

and pharmaceutically acceptable salts thereof;

wherein:

A-J is

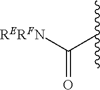

### and Q-G is

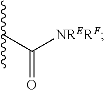

or A-J is

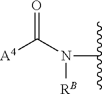

### and Q-G is

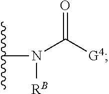

or A-J is

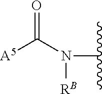

### and Q-G is

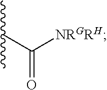

or A-J is

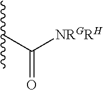

### and Q-G is

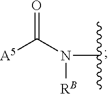

each E is separately selected from the group consisting of —CR^{10a}— and N (nitrogen);

each R^{10a} is separately selected from the group consisting of H (hydrogen), halogen, cyano, C₁-C₆ alkyl optionally substituted with up to five fluoro, C₁-C₆ alkoxy optionally substituted with up to five fluoro, C₂-C₆ alkenyl optionally substituted with up to five fluoro, C₂-C₆ alkynyl optionally substituted with up to five fluoro, C₃-C₇ cycloalkyl optionally substituted with up to five fluoro, and C₃-C₇ cycloalkenyl optionally substituted with up to five fluoro;

R^{10b} is selected from the group consisting of R^{10bb}, H (hydrogen), halogen, cyano, C₁-C₆ alkyl optionally substituted with up to five fluoro, C₁-C₆ alkoxy optionally substituted with up to five fluoro, C₂-C₆ alkenyl optionally substituted with up to five fluoro, C₂-C₆ alkynyl optionally substituted with up to five fluoro, C₃-C₇ cycloalkyl optionally substituted with up to five fluoro, and C₃-C₇ cycloalkenyl optionally substituted with up to five fluoro;

R^{10c} is selected from the group consisting of H (hydrogen), R^{U}SO₂—, R^{U}C(═O)—, C₁-C₆ alkyl optionally substituted with up to five fluoro, and C₃-C₇ cycloalkyl optionally substituted with up to five fluoro, or R^{10c} is R^{10cc} when R^{10b} is R^{10bb};

R^{10cc} and R^{10bb} together with the atoms to which they are attached is a five-membered heteroaryl optionally substituted with one or more substituents each separately selected from the group consisting of halogen, cyano, C₁-C₆ alkyl optionally substituted with up to five fluoro, C₁-C₆ alkoxy optionally substituted with up to five fluoro, C₁-C₆ alkylC(═O)— and C₃-C₇ cycloalkylC(═O)—;

A⁴ is selected from the group consisting of C₃-C₇ cycloalkenyl, C₃-C₇ cycloalkyl, C₁-C₆ alkyl, C₁-C₆ heteroalkyl, C₂-C₆ alkenyl, C₁-C₆ alkoxy, —(CH₂)ₘNR^{P}R^{L} heterocycle, polycyclic heterocyclyl, aryl, and heteroaryl, said C₃-C₇ cycloalkenyl, C₃-C₇ cycloalkyl, C₁-C₆ alkyl, C₁-C₆ heteroalkyl, C₂-C₆ alkenyl, heterocycle, polycyclic heterocyclyl, aryl, and heteroaryl, each optionally substituted with one or more substituents selected from the group consisting of R¹, R², and R³;

G⁴ is selected from the group consisting of polycyclic heterocyclyl, aryl, and heteroaryl, each optionally substituted with one or more substituents selected from the group consisting of R⁴, R⁵, and R⁶;

A⁵ is selected from the group consisting of polycyclic heterocyclyl, aryl and heteroaryl, each optionally substituted with one or more substituents selected from the group consisting of R¹, R², and R³;

each R^{B} is separately selected from the group consisting of hydrogen, an optionally substituted C₁-C₆ alkyl, an optionally substituted C₂-C₆ alkenyl, and an optionally substituted C₃-C₇ cycloalkyl;

each —NR^{E}R^{F} is separately selected, wherein each R^{E} is independently selected from the group consisting of hydrogen and an optionally C₁-C₆ alkyl, and each RF is independently selected from the group consisting of aryl and heteroaryl, said aryl and heteroaryl in the definition of R^{F} are each optionally substituted with halogen, cyano, C₁-C₆ alkyl, C₁-C₆ alkoxy, C₃-C₇ cycloalkyl, C₃-C₇ cycloalkenyl, —C(═O)NR^{N}R^{O}, —OC(═O)NR^{N}R^{O}, —NHC(═O)NR^{N}R^{O}, —O(CH₂)_{q}NR^{N}R^{O}, —NH(CH₂)_{q}NR^{N}R^{O}—(CH₂)ₚNR^{N}R^{O}, an optionally substituted aryl and an optionally substituted heteroaryl, and said aryl and heteroaryl in the definition of R^{F} are each further optionally fused with an optionally substituted nonaromatic heterocycle or an optionally substituted nonaromatic carbocycle;

R^{G} is selected from the group consisting of C₁-C₆ alkyl, C₃-C₆ cycloalkyl, C₃-C₈ cycloalkenyl, C₁-C₆ heteroalkyl, C₁-C₆ heteroalkenyl, C₁-C₆ heteroalkynyl, heterocycle, aryl, and heteroaryl, each optionally substituted with one or more substituents selected from the group consisting of R⁴, R⁵, and R⁶, said aryl and heteroaryl in the definition of R^{G} are each further optionally fused with an optionally substituted nonaromatic heterocycle or an optionally substituted nonaromatic carbocycle, or R^{G} is —OR^{L} or —NR^{P}R^{L};

R^{H} is selected from the group consisting of hydrogen, C₁-C₆ alkyl, C₂-C₆ alkenyl, C₃-C₇ cycloalkyl, and C₁-C₃ haloalkyl, or —NR^{G}R^{H} is an optionally substituted non-aromatic heterocycle linked through a ring nitrogen atom;

each R¹ is separately selected from the group consisting of halogen, cyano, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, optionally substituted C₃-C₇ cycloalkenyl, an optionally substituted C₁-C₆ haloalkyl, an optionally substituted C₁-C₆ heteroalkyl, an optionally substituted aryl, and an optionally substituted heteroaryl;

each R² is separately selected from the group consisting of halogen, —O(CH₂)ₘOR^{I}, —(CH₂)ₘOR^{I}, —NR^{J}R^{K}, —(CH₂)ₘSR^{I}, —C(═O)R^{L}, —(CH₂)ₘR^{L}, an optionally substituted C₁-C₆ alkyl, an optionally substituted C₁-C₆ alkoxy, an optionally substituted C₂-C₆ alkenyl, an optionally substituted C₁-C₆ haloalkyl, an optionally substituted C₁-C₆ heteroalkyl, and an optionally substituted C₃-C₇ cycloalkyl where said C₃-C₇ cycloalkyl is further optionally fused with aryl or heteroaryl;

each R³ is separately selected from the group consisting of halogen, —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, —(CH₂)ₘS(O)₀₋₂R^{M}, —(CH₂)ₘNHS(O)₀₋₂R^{M}, —(CH₂)ₘNO₂, —(CH₂)ₘCN, —(CH₂)ₘR^{P}, C₁-C₆ alkyl C₁-C₆ alkoxy, C₂-C₆ alkenyl, C₂-C₆ alkynyl, C₃-C₇ cycloalkyl, C₁-C₆ haloalkyl, heterocycle, aryl, polycyclic heterocyclyl, and heteroaryl, said heterocycle, aryl polycyclic heterocyclyl, and heteroaryl in the definition of R³ are each optionally substituted with halogen, hydroxy, cyano, C₁-C₆ alkyl, C₁-C₆ haloalkyl, C₁-C₆ alkoxy, —C(═O)OR^{M}, or —NR^{J}R^{K};

each R⁴ is separately selected from the group consisting of halogen, cyano, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted C₁-C₆ haloalkyl, an optionally substituted C₁-C₆ heteroalkyl, an optionally substituted aryl, and an optionally substituted heteroaryl;

each R⁵ is separately selected from the group consisting of halogen, —O(CH₂)ₘOR^{I}, —(CH₂)ₘOR^{I}, —NR^{J}R^{K}, —(CH₂)ₘSR^{I}, —(CH₂)ₘC(═O)R^{L}, —(CH₂)ₘR^{L}, an optionally substituted C₁-C₆ alkyl, an optionally substituted C₁-C₆ alkoxy, an optionally substituted C₂-C₆ alkenyl, an optionally substituted C₃-C₇ cycloalkyl, an optionally substituted C₁-C₆ haloalkyl, and an optionally substituted C₁-C₆ heteroalkyl;

each R⁶ is separately selected from the group consisting of halogen, —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, (CH₂)ₘS(O)₀₋₂R^{M}, —(CH₂)ₘNHS(O)₀₋₂R^{M}, —(CH₂)ₘNO₂, —(CH₂)ₘCN, —(CH₂)ₘR^{P}, C₁-C₆ alkyl, C₁-C₆ alkoxy, C₂-C₆ alkenyl, C₂-C₆ alkynyl, C₃-C₇ cycloalkyl, C₁-C₆ haloalkyl, C₁-C₆ heteroalkyl, heterocycle, aryl, polycyclic heterocyclyl, and heteroaryl, said heterocycle, aryl, polycyclic heterocyclyl, and heteroaryl in the definition of R⁶ are each optionally substituted with halogen, cyano, C₁-C₆ alkyl, C₁-C₆ haloalkyl, C₁-C₆ alkoxy, —C(═O)OR^{M}, or —NR^{J}R^{K};

each R^{I} is separately selected from the group consisting of hydrogen, C₁-C₆ alkyl, C₂-C₄ alkenyl, C₂-C₄ alkynyl, C₃-C₇ cycloalkyl, C₁-C₆ haloalkyl, C₁-C₆ heteroalkyl, and C₁-C₆ heterohaloalkyl;

each —NR^{J}R^{K} is separately selected, wherein R^{J} and R^{K} are each independently selected from the group consisting of hydrogen, C₁-C₆ alkyl optionally substituted with up to 5 fluoro, —(CH₂)ₘOR^{JA}, —(CH₂)ₘNR^{JB}R^{JC}, —(CH₂)ₘR^{R}, C₃-C₇ cycloalkyl, heterocycle, aryl and heteroaryl, said C₃-C₇ cycloalkyl, heterocycle, aryl and heteroaryl in the definition of R^{J} and R^{K} are each independently optionally substituted with one or more substituents selected from the group consisting of halo, C₁-C₆ alkyl, C₁-C₆ haloalkyl, C₁-C₆ alkoxy, aryl and heteroaryl, said aryl and heteroaryl substituent off of R^{J} and R^{K} are each optionally substituted with one or more halo, C₁-C₆ alkyl, C₁-C₆ haloalkyl, C₁-C₆ alkoxy, or —(CH₂)ₘNR^{KA}R^{KB}; or —NR^{J}R^{K} is an optionally substituted non-aromatic heterocycle linked through a ring nitrogen atom;

each R^{JA} is independently selected from the group consisting of hydrogen, C₁-C₆ alkyl, and C₁-C₆ haloalkyl;

each —NR^{JB}R^{JC} is separately selected, wherein R^{JB} and R^{JC} are each independently selected from the group consisting of hydrogen, C₁-C₆ alkyl, and C₁-C₆ haloalkyl;

each —NR^{KA}R^{KB} is separately selected, wherein R^{KA} and R^{KB} are each independently selected from the group consisting of hydrogen, C₁-C₆ alkyl, and C₁-C₆ haloalkyl;

each R^{M} is independently selected from the group consisting of hydrogen, an optionally substituted C₁-C₆ alkyl, an optionally substituted C₂-C₄ alkenyl, an optionally substituted C₂-C₄ alkynyl, an optionally substituted C₃-C₇ cycloalkyl, an optionally substituted C₃-C₇ cycloalkenyl, and —(CH₂)ₘR^{P};

each —NR^{N}R^{O} is separately selected, wherein R^{N} and R^{O} are each independently selected from the group consisting of hydrogen, —(CH₂)ₘNR^{NA}R^{NB}, aryl and heteroaryl, said aryl and heteroaryl in the definition of R^{N} and R^{O} are each independently optionally substituted with one or more substituents selected from the group consisting of —(CH₂)ₘNR^{OA}R^{OB}, halo, C₁-C₆ alkyl, C₁-C₆ haloalkyl, C₁-C₆ alkoxy, aryl and heteroaryl, said aryl and heteroaryl substituent off of R^{N} and R^{O} are each optionally substituted with one or more halo, C₁-C₆ alkyl, C₁-C₆ haloalkyl, C₁-C₆ alkoxy, or —NR^{NA}R^{NB}

each —NR^{NA}R^{NB} is separately selected, wherein R^{NA} and R^{NB} are each independently selected from the group consisting of hydrogen, C₁-C₆ alkyl, and C₁-C₆ haloalkyl;

each —NR^{A}R^{O}B is separately selected, wherein R^{OA} and R^{OB} are each independently selected from the group consisting of hydrogen, C₁-C₆ alkyl, and C₁-C₆ haloalkyl;

each R^{P} is independently selected from the group consisting of hydrogen and C₁-C₆ alkyl;

each R^{L} is independently selected from the group consisting of C₃-C₇ cycloalkyl, optionally substituted C₁-C₆ alkyl, optionally substituted C₁-C₆ alkoxy, —(CH₂)ₘOR^{LA}, —(CH₂)ₘNR^{LB}R^{LC}, aryl and heteroaryl, said aryl and heteroaryl in the definition of R^{L} are each independently optionally substituted with one or more substituents selected from the group consisting of halo, C₁-C₆ alkyl, C₁-C₆ haloalkyl, C₁-C₆ alkoxy, —(CH₂)ₘNR^{LD}R^{LE}, aryl and heteroaryl, said aryl and heteroaryl substituent off of R^{L} are each optionally substituted with one or more halo, C₁-C₆ alkyl, C₁-C₆ haloalkyl, C₁-C₆ alkoxy, or —(CH₂)ₘNR^{LF}R^{LG};

each R^{LA} is independently selected from the group consisting of hydrogen, C₁-C₆ alkyl, and C₁-C₆ haloalkyl;

R^{LB} and R^{LC} are each independently selected from the group consisting of hydrogen, C₁-C₆ alkyl, C₁-C₆ haloalkyl, and C₁-C₆ heteroalkenyl, said C₁-C₆ alkyl, C₁-C₆ haloalkyl, and C₁-C₆ heteroalkenyl each optionally substituted with one or more halogen, cyano, or —(CH₂)ₘC(═O)OH; or —NR^{LB}R^{LC} is an optionally substituted non-aromatic heterocycle linked through a ring nitrogen atom;

each —NR^{LD}R^{LE} is separately selected, wherein R^{LD} and R^{LE} are each independently selected from the group consisting of hydrogen, aryl, heteroaryl, and optionally substituted C₁-C₆ alkyl, said aryl and heteroaryl in the definition of R^{LD} and R^{LE} are each optionally substituted with C₁-C₆ alkyl or C₁-C₆ alkoxy; or —NR^{LD}R^{LE} is an optionally substituted non-aromatic heterocycle linked through a ring nitrogen atom;

each —NR^{LF}R^{LG} is separately selected, wherein R^{LF} and R^{LG} are each independently selected from the group consisting of hydrogen, and C₁-C₆ alkyl; or —NR^{LF}R^{LG} is an optionally substituted non-aromatic heterocycle linked through a ring nitrogen atom;

R^{R} is selected from the group consisting of C₁-C₆ alkyl, an optionally substituted aryl, and an optionally substituted heteroaryl;

R^{U} is selected from the group consisting of C₃-C₇ cycloalkyl C₁-C₆ alkyl optionally substituted with up to 5 fluoro, and an optionally substituted heteroaryl;

each m is independently 0, 1, 2, or 3;

each p is independently 0, 1, 2, 3, 4, 5, or 6; and

each q is independently 1, 2, 3, 4, 5, or 6.

Some embodiments disclosed herein provide a compound of Formula VIII having the formula VIIIa:

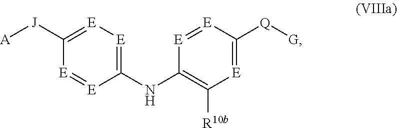

and pharmaceutically acceptable salts thereof.

Some embodiments disclosed herein provide a compound of Formula VIIIa having the formula VIIIaa:

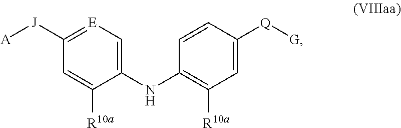

and pharmaceutically acceptable salts thereof, and having the formula VIIIab:

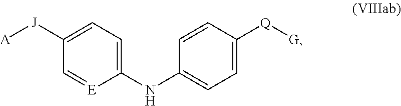

and pharmaceutically acceptable salts thereof.

Some embodiments disclosed herein provide a compound of Formula VIIIaa, wherein A-J is

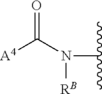

### and Q-G is

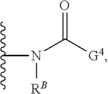

A⁴ is selected from the group consisting of aryl and heteroaryl, where at least one atom forming the heteroaryl aromatic ring is a N (nitrogen), and said aryl and heteroaryl are each optionally substituted with one or more substituents selected from the group consisting of R¹, R², and R³, and G⁴ is selected from the group consisting of aryl and heteroaryl, where at least one atom forming the heteroaryl aromatic ring is a N (nitrogen), and said aryl and heteroaryl are each optionally substituted with one or more substituents selected from the group consisting of R⁴, R⁵, and R⁶.

Some embodiments disclosed herein provide a compound of Formula VIII having the formula VIIIb:

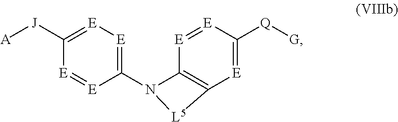

and pharmaceutically acceptable salts thereof, wherein L⁵ is E¹=E¹ or E²-E², each E¹ is separately selected from the group consisting of —CR^{10dd}— and N (nitrogen), each R^{10dd} is separately selected from the group consisting of H (hydrogen), halogen, cyano, C₁-C₆ alkyl optionally substituted with up to five fluoro, C₁-C₆ alkoxy optionally substituted with up to five fluoro, C₁-C₆ alkylC(═O)— and C₃-C₇ cycloalkylC(═O)—, each E² is separately selected from the group consisting of —CR⁷R⁸— and NR⁹, R⁷ and R⁸ are each independently selected from the group consisting of hydrogen, —OH, and C₁-C₆ alkyl optionally substituted with up to five fluoro, or optionally CR⁷R⁸ is —C(═O)—, and, R⁹ is selected from the group consisting of hydrogen, C₃-C₇cycloalkylC(O)— and C₁-C₆ alkylC(O)—, and C₁-C₆ alkyl optionally substituted with up to five fluoro.

Some embodiments disclosed herein provide a compound of Formula VIIIb having the formula VIIIbb;

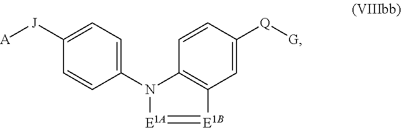

and pharmaceutically acceptable salts thereof, wherein E^{1A} is N (nitrogen) and E^{1B} is —CH—, or E^{1A} is —CH— and E^{1B} is —CH—, or E^{1A} is —CH— and E^{1B} is N (nitrogen); and A-J is

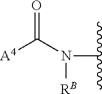

### and Q-G is

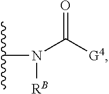

### or A-J is

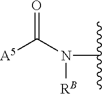

### and Q-G is

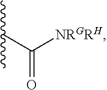

### or A-J is

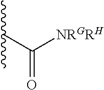

### and Q-G is

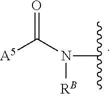

Some embodiments disclosed herein provide a compound of Formula VIIIbb having the formula VIIIbbb:

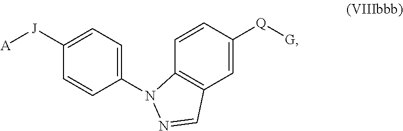

and pharmaceutically acceptable salts thereof, wherein A-J is

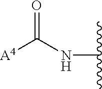

### and Q-G is

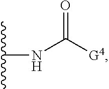

### or A-J is

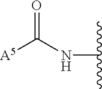

### and Q-G is

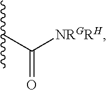

### or A-J is

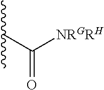

### and Q-G is

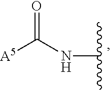

A⁴ is selected from the group consisting of C₅-C₇ cycloalkenyl, C₃-C₆ cycloalkyl, C₁-C₆ alkyl, C₁-C₆ heteroalkyl, C₂-C₆ alkenyl, —NR^{P}R^{L}, heterocycle, aryl, and heteroaryl, said C₅-C₇ cycloalkenyl, C₃-C₆ cycloalkyl, C₁-C₆ alkyl, C₁-C₆ heteroalkyl, C₂-C₆ alkenyl, heterocycle, aryl, and heteroaryl, each optionally substituted with one or more substituents selected from the group consisting of R¹, R², and R³, each R¹ is separately selected from the group consisting of halogen, cyano, an optionally substituted C₁-C₆ alkyl, an optionally substituted C₁-C₆ alkoxy, an optionally substituted C₁-C₆ heteroalkyl, an optionally substituted aryl, and an optionally substituted heteroaryl, each R² is separately selected from the group consisting of —O(CH₂)ₘOR^{I}, —(CH₂)ₘOR^{I}, —NR^{J}R^{K}, —(CH₂)ₘSR^{I}, —C(═O)R^{L}, and —(CH₂)ₘR^{L}, each R³ is separately selected from the group consisting of —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, —(CH₂)ₘS(O)₀₋₂R^{M}, —(CH₂)ₘNHS(O)₀₋₂R^{M}, —(CH₂)ₘNO₂, —(CH₂)ₘCN, —(CH₂)ₘR^{P}, heterocycle, aryl, polycyclic heterocyclyl, and heteroaryl, said heterocycle, aryl polycyclic heterocyclyl, and heteroaryl in the definition of R³ are each optionally substituted with halogen, hydroxy, cyano, C₁-C₆ alkyl, C₁-C₆ haloalkyl, C₁-C₆ alkoxy, —C(═O)OR^{M}, or —NR^{J}R^{K}, G⁴ is selected from the group consisting of aryl, and heteroaryl, each optionally substituted with one or more substituents selected from the group consisting of R⁴, R⁵, and R⁶, each R⁴ is separately selected from the group consisting of halogen, cyano, an optionally substituted C₁-C₆ alkyl, and an optionally substituted C₁-C₆ heteroalkyl, each R⁵s is separately selected from the group consisting of halogen, —(CH₂)ₘOH, —NR^{J}R^{K}, and —(CH₂)ₘC(═O)R^{L}, each R^{L} is independently selected from the group consisting of —OH, C₁-C₆ alkyl, and C₁-C₆ alkoxy, each R⁶ is separately selected from the group consisting of —NR^{L}C(═O)R^{M}, —NR^{L}C(═O)OR^{M}, —NR^{L}C(═O)NR^{N}R^{O}, —NR^{N}R^{O}, —(CH₂)ₘR^{P}, heterocycle, aryl, polycyclic heterocyclyl, and heteroaryl, said heterocycle, aryl, polycyclic heterocyclyl, and heteroaryl in the definition of R⁶ are each optionally substituted with halogen, cyano, C₁-C₆ alkyl, C₁-C₆ haloalkyl, C₁-C₆ alkoxy, —C(═O)OR^{M}, or —NR^{J}R^{K}, R^{G} within the definition of —NR^{G}R^{H} is heteroaryl optionally substituted with one or more substituents selected from the group consisting of R⁴, R⁵, and R⁶, and R^{H} within the definition of —NR^{G}R^{H} is hydrogen.

Some embodiments disclosed herein provide a compound of Formula IX:

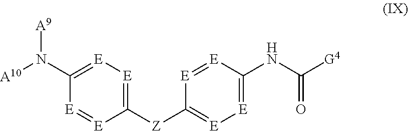

and pharmaceutically acceptable salts thereof;

wherein:

Z is

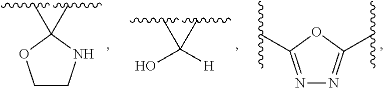

### or —C(=E³)-;

each E is separately selected from the group consisting of —CR^{10a}— and N (nitrogen);

each R^{10a} is separately selected from the group consisting of H (hydrogen), halogen, C₁-C₆ alkyl optionally substituted with up to five fluoro, and C₁-C₆ alkoxy optionally substituted with up to five fluoro;

E³ is O (oxygen), N—NHR^{Q} or N—OR^{Q} where R^{Q} in the definition of E³ is selected from the group consisting of hydrogen, C₁-C₆ alkyl, C₂-C₆ alkenyl, —(CH₂)ₘR^{RA}, and —C(═O)(CH₂)ₘR^{RA};

R^{RA} is selected from the group consisting of C₁-C₆ alkyl, aryl, and heteroaryl;

A⁹ is hydrogen or C₁-C₆ alkyl;

A¹⁰ is selected from the group consisting of C₁-C₆ alkyl, C₂-C₆ alkenyl, —C(═O)R^{A}, —C(═O)C(═O)R^{A}, —(CH₂)R^{B}, —(CH₂)OR^{B};

R^{A} is selected from the group consisting of C₁-C₆ alkyl, C₃-C₇ cycloalkyl, heterocycle, polycyclic heterocyclyl, aryl and heteroaryl, each optionally substituted with one or more substituents selected from the group consisting of R¹, R², and R³;

R^{B} is selected from the group consisting of hydrogen, C₁-C₆ alkyl, C₁-C₆ alkoxy, C₂-C₆ alkenyl, C₃-C₇ cycloalkyl, and heteroaryl;

G⁴ is selected from the group consisting of polycyclic heterocyclyl, aryl, and heteroaryl, each optionally substituted with one or more substituents selected from the group consisting of R⁴, R⁵, and R⁶;

each R¹ is separately selected from the group consisting of halogen, cyano, C₁-C₆ heteroalkyl, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, optionally substituted C₃-C₇ cycloalkenyl, an optionally substituted C₁-C₆ haloalkyl, an optionally substituted aryl, and an optionally substituted heteroaryl;

each R² is separately selected from the group consisting of halogen, —O(CH₂)ₘOR^{I}, —(CH₂)ₘOR^{I}, —NR^{J}R^{K}, —(CH₂)ₘSR^{I}, —C(═O)R^{L}, —(CH₂)ₘR^{L}, C₁-C₆ heteroalkyl, an optionally substituted C₁-C₆ alkyl, an optionally substituted C₁-C₆ alkoxy, an optionally substituted C₂-C₆ alkenyl, an optionally substituted C₁-C₆ haloalkyl, and an optionally substituted C₃-C₇ cycloalkyl where said C₃-C₇ cycloalkyl is further optionally fused with aryl or heteroaryl;

each R³ is separately selected from the group consisting of halogen, —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, —(CH₂)ₘS(O)₀₋₂R^{M}, —(CH₂)ₘNHS(O)₀₋₂R^{M}, —(CH₂)ₘCN, —(CH₂)ₘR^{P}, C₁-C₆ alkyl C₁-C₆ alkoxy, C₂-C₆ alkenyl, C₂-C₆ alkynyl, C₃-C₇ cycloalkyl, C₁-C₆ haloalkyl, heterocycle, aryl, polycyclic heterocyclyl, and heteroaryl, said heterocycle, aryl polycyclic heterocyclyl, and heteroaryl in the definition of R³ are each optionally substituted with halogen, hydroxy, cyano, C₁-C₆ alkyl, C₁-C₆ haloalkyl, C₁-C₆ alkoxy, —C(═O)OR^{M}, or —NR^{J}R^{K};

each R⁴ is separately selected from the group consisting of halogen, cyano, C₁-C₆ heteroalkyl, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted C₁-C₆ haloalkyl, an optionally substituted aryl, and an optionally substituted heteroaryl;

each R⁵ is separately selected from the group consisting of halogen, —O(CH₂)ₘOR^{I}, —(CH₂)ₘOR^{I}, —NR^{J}R^{K}, —(CH₂)ₘSR^{I}, —(CH₂)ₘC(═O)R^{L}, —(CH₂)ₘR^{L}, C₁-C₆ heteroalkyl, an optionally substituted C₁-C₆ alkyl, an optionally substituted C₁-C₆ alkoxy, an optionally substituted C₂-C₆ alkenyl, an optionally substituted C₃-C₇ cycloalkyl, and an optionally substituted C₁-C₆ haloalkyl;

each R⁶ is separately selected from the group consisting of halogen, —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, (CH₂)ₘS(O)₀₋₂R^{M}, —(CH₂)ₘNHS(O)₀₋₂R^{M}, —(CH₂)ₘCN, —(CH₂)ₘR^{P}, C₁-C₆ alkyl, C₁-C₆ alkoxy, C₂-C₆ alkenyl, C₂-C₆ alkynyl, C₃-C₇ cycloalkyl, C₁-C₆ haloalkyl, C₁-C₆ heteroalkyl, heterocycle, aryl, polycyclic heterocyclyl, and heteroaryl, said heterocycle, aryl, polycyclic heterocyclyl, and heteroaryl in the definition of R⁶ are each optionally substituted with halogen, cyano, C₁-C₆ alkyl, C₁-C₆ haloalkyl, C₁-C₆ alkoxy, —C(═O)OR^{M}, or —NR^{J}R^{K};

R^{G} is selected from the group consisting of C₁-C₆ alkyl, C₃-C₆ cycloalkyl, C₃-C₈ cycloalkenyl, C₁-C₆ heteroalkyl, C₁-C₆ heteroalkenyl, C₁-C₆ heteroalkynyl, heterocycle, aryl, and heteroaryl, each optionally substituted with one or more substituents selected from the group consisting of R⁴, R⁵, and R⁶, said aryl and heteroaryl in the definition of R^{G} are each further optionally fused with an optionally substituted nonaromatic heterocycle or an optionally substituted nonaromatic carbocycle, or R^{G} is —OR^{L} or —NR^{P}R^{L};

each R^{I} is separately selected from the group consisting of hydrogen, C₁-C₆ alkyl, C₂-C₄ alkenyl, C₂-C₄ alkynyl, C₃-C₇ cycloalkyl, C₁-C₆ haloalkyl, C₁-C₆ heteroalkyl, and C₁-C₆ heterohaloalkyl;

each —NR^{J}R^{K} is separately selected, wherein R^{J} and R^{K} are each independently selected from the group consisting of hydrogen, C₁-C₆ alkyl optionally substituted with up to 5 fluoro, —(CH₂)ₘOR^{JA}, —(CH₂)ₘNR^{JB}R^{JC}, —(CH₂)ₘR^{R}, C₃-C₇ cycloalkyl, heterocycle, aryl and heteroaryl, said C₃-C₇ cycloalkyl, heterocycle, aryl and heteroaryl in the definition of R^{J} and R^{K} are each independently optionally substituted with one or more substituents selected from the group consisting of halo, C₁-C₆ alkyl, C₁-C₆ haloalkyl, C₁-C₆ alkoxy, aryl and heteroaryl, said aryl and heteroaryl substituent off of R^{J} and R^{K} are each optionally substituted with one or more halo, C₁-C₆ alkyl, C₁-C₆ haloalkyl, C₁-C₆ alkoxy, or —(CH₂)ₘNR^{KA}R^{KB}; or —NR^{J}R^{K} is an optionally substituted non-aromatic heterocycle linked through a ring nitrogen atom;

each R^{JA} is independently selected from the group consisting of hydrogen, C₁-C₆ alkyl, and C₁-C₆ haloalkyl;

each —NR^{JB}R^{JC} is separately selected, wherein R^{JB} and R^{JC} are each independently selected from the group consisting of hydrogen, C₁-C₆ alkyl, and C₁-C₆ haloalkyl;

each —NR^{KA}R^{KB} is separately selected, wherein R^{KA} and R^{KB} are each independently selected from the group consisting of hydrogen, C₁-C₆ alkyl, and C₁-C₆ haloalkyl;

each R^{M} is independently selected from the group consisting of hydrogen, an optionally substituted C₁-C₆ alkyl, an optionally substituted C₂-C₄ alkenyl, an optionally substituted C₂-C₄ alkynyl, an optionally substituted C₃-C₇ cycloalkyl, an optionally substituted C₃-C₇ cycloalkenyl, and —(CH₂)ₘR^{P};

each —NR^{N}R^{O} is separately selected, wherein R^{N} and R^{O} are each independently selected from the group consisting of hydrogen, —(CH₂)ₘNR^{NA}R^{NB}, aryl and heteroaryl, said aryl and heteroaryl in the definition of R^{N} and R^{O} are each independently optionally substituted with one or more substituents selected from the group consisting of —(CH₂)ₘNR^{OA}R^{OB}, halo, C₁-C₆ alkyl, C₁-C₆ haloalkyl, C₁-C₆ alkoxy, aryl and heteroaryl, said aryl and heteroaryl substituent off of R^{N} and R^{O} are each optionally substituted with one or more halo, C₁-C₆ alkyl, C₁-C₆ haloalkyl, C₁-C₆ alkoxy, or —NR^{NA}R^{NB},

each —NR^{NA}R^{NB} is separately selected, wherein R^{NA} and R^{NB} are each independently selected from the group consisting of hydrogen, C₁-C₆ alkyl, and C₁-C₆ haloalkyl;

each —NR^{OA}R^{OB} is separately selected, wherein R^{OA} and R^{OB} are each independently selected from the group consisting of hydrogen, C₁-C₆ alkyl, and C₁-C₆ haloalkyl;

R^{P} is selected from the group consisting of hydrogen and C₁-C₆ alkyl;

each R^{L} is independently selected from the group consisting of C₃-C₇ cycloalkyl, optionally substituted C₁-C₆ alkyl, optionally substituted C₁-C₆ alkoxy, —(CH₂)ₘOR^{LA}, —(CH₂)ₘNR^{LB}R^{LC}, aryl and heteroaryl, said aryl and heteroaryl in the definition of R^{L} are each independently optionally substituted with one or more substituents selected from the group consisting of halo, C₁-C₆ alkyl, C₁-C₆ haloalkyl, C₁-C₆ alkoxy, —(CH₂)ₘNR^{LD}R^{LE}, aryl and heteroaryl, said aryl and heteroaryl substituent off of R^{L} are each optionally substituted with one or more halo, C₁-C₆ alkyl, C₁-C₆ haloalkyl, C₁-C₆ alkoxy, or —(CH₂)ₘNR^{LF}R^{LG}

each R^{LA} is independently selected from the group consisting of hydrogen, C₁-C₆ alkyl, and C₁-C₆ haloalkyl;

R^{LB} and R^{LC} are each independently selected from the group consisting of hydrogen, C₁-C₆ alkyl, C₁-C₆ haloalkyl, and C₁-C₆ heteroalkenyl, said C₁-C₆ alkyl, C₁-C₆ haloalkyl, and C₁-C₆ heteroalkenyl each optionally substituted with one or more halogen, cyano, or —(CH₂)ₘC(═O)OH; or —NR^{LB}R^{LC} is an optionally substituted non-aromatic heterocycle linked through a ring nitrogen atom;

each —NR^{LD}R^{LE} is separately selected, wherein R^{LD} and R^{LE} are each independently selected from the group consisting of hydrogen, aryl, heteroaryl, and optionally substituted C₁-C₆ alkyl, said aryl and heteroaryl in the definition of R^{LD} and

R^{LE} are each optionally substituted with C₁-C₆ alkyl or C₁-C₆ alkoxy; or —NR^{LD}R^{LE} is an optionally substituted non-aromatic heterocycle linked through a ring nitrogen atom;

each —NR^{LF}R^{LG} is separately selected, wherein R^{LF} and R^{LG} are each independently selected from the group consisting of hydrogen, and C₁-C₆ alkyl; or —NR^{LF}R^{LG} is an optionally substituted non-aromatic heterocycle linked through a ring nitrogen atom;

R^{R} is selected from the group consisting of C₁-C₆ alkyl, an optionally substituted aryl, and an optionally substituted heteroaryl; and

each m is independently 0, 1, 2, or 3.

In some embodiments, a compound of Formula IX is not selected from the group consisting of:

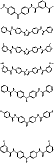

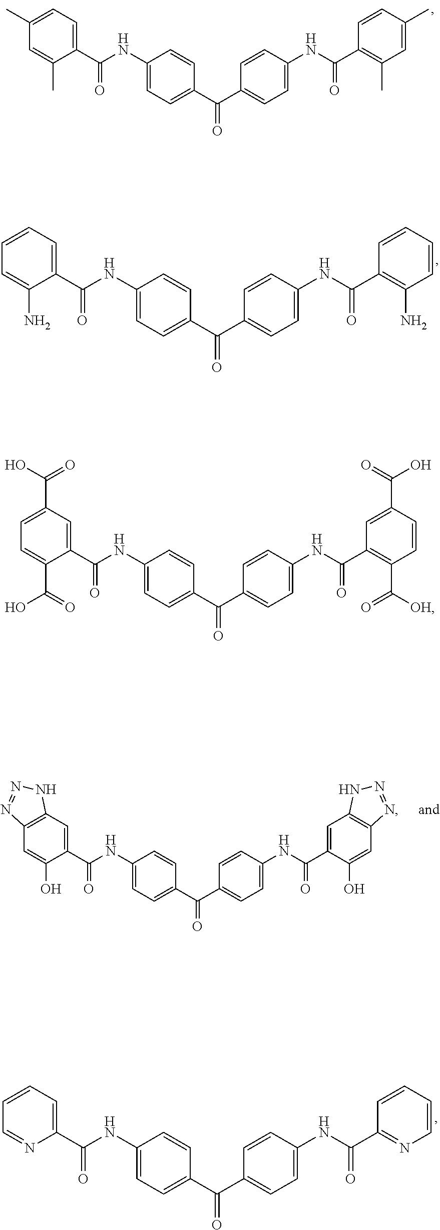

Some embodiments disclosed herein provide a compound of Formula IX having the formula IXa:

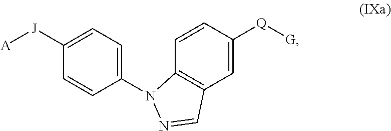

and pharmaceutically acceptable salts thereof, wherein Z is

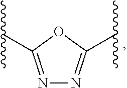 [SMILES: *c1nnc(*)o1]

or —C(═O)—, R^{A} is selected from the group consisting of heterocycle, polycyclic heterocyclyl, aryl and heteroaryl, each substituted with one or more substituents selected from the group consisting of R¹, R², and R³, each R¹ is separately selected from the group consisting of chloro, cyano, C₁-C₆ alkyl, C₁-C₆ alkoxy, C₂-C₆ alkenyl, C₂-C₆ alkynyl, C₃-C₇ cycloalkyl, C₃-C₇ cycloalkenyl, and C₁-C₆ haloalkyl, each R² is separately selected from the group consisting of —O(CH₂)ₘOR^{I}, —(CH₂)ₘOR^{I}, —NR^{J}R^{K}, —(CH₂)ₘSR^{I}, —C(═O)R^{L}, and —(CH₂)ₘR^{L}, and each R³ is separately selected from the group consisting of —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, —(CH₂)ₘS(O)₀₋₂R¹—(CH₂)ₘNHS(O)₀₋₂R^{M}, —(CH₂)ₘCN, —(CH₂)ₘR^{P}, heterocycle, aryl, polycyclic heterocyclyl, and heteroaryl, said heterocycle, aryl polycyclic heterocyclyl, and heteroaryl in the definition of R³ are each optionally substituted with halogen, hydroxy, cyano, C₁-C₆ alkyl, C₁-C₆ haloalkyl, C₁-C₆ alkoxy, —C(═O)OR^{M}, or —NR^{J}R^{K}.

Some embodiments disclosed herein provide a compound of Formula IXa having the formula IXaa:

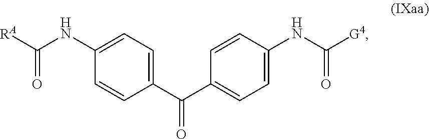

and pharmaceutically acceptable salts thereof, wherein G⁴ is selected from the group consisting of polycyclic heterocyclyl, aryl, and heteroaryl, each substituted with one or more substituents selected from the group consisting of R⁴, R⁵, and R⁶, each R⁴ is separately selected from the group consisting of chloro, cyano, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted C₁-C₆ haloalkyl, an optionally substituted C₁-C₆ heteroalkyl, an optionally substituted aryl, and an optionally substituted heteroaryl, each R⁵ is separately selected from the group consisting of —O(CH₂)ₘOR^{I}, —(CH₂)ₘOR^{I}, —NR^{J}R^{K}, —(CH₂)ₘSR^{I}, —(CH₂)ₘC(═O)R^{L}, —(CH₂)ₘR^{L}, an optionally substituted C₁-C₆ alkyl, an optionally substituted C₁-C₆ alkoxy, an optionally substituted C₂-C₆ alkenyl, an optionally substituted C₃-C₇ cycloalkyl, an optionally substituted C₁-C₆ haloalkyl, and an optionally substituted C₁-C₆ heteroalkyl, each R⁶ is separately selected from the group consisting of —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, —(CH₂)ₘS(O)₀₋₂R^{M}, —(CH₂)ₘNHS(O)₀₋₂R^{M}, —(CH₂)ₘNO₂, —(CH₂)ₘCN, —(CH₂)ₘR^{P}, C₁-C₆ alkyl, C₁-C₆ alkoxy, C₂-C₆ alkenyl, C₂-C₆ alkynyl, C₃-C₇ cycloalkyl, C₁-C₆ haloalkyl, C₁-C₆ heteroalkyl, heterocycle, aryl, polycyclic heterocyclyl, and heteroaryl, said heterocycle, aryl, polycyclic heterocyclyl, and heteroaryl in the definition of R⁶ are each optionally substituted with halogen, cyano, C₁-C₆ alkyl, C₁-C₆ haloalkyl, C₁-C₆ alkoxy, —C(═O)OR^{M}, or —NR^{J}R^{K}.

Some embodiments disclosed herein provide a compound of Formula IXa, wherein each R¹ is separately selected from the group consisting of cyano, an optionally substituted C₁-C₆ alkyl, an optionally substituted C₁-C₆ alkoxy, an optionally substituted C₁-C₆ heteroalkyl, an optionally substituted aryl, and an optionally substituted heteroaryl, each R² is separately selected from the group consisting of —O(CH₂)ₘOR^{I}, —(CH₂)ₘOR^{I}, —NR^{J}R^{K}, —(CH₂)ₘSR^{I}, —C(═O)R^{L}, and —(CH₂)ₘR^{L}, each R³ is separately selected from the group consisting of —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, —(CH₂)ₘS(O)₀₋₂R^{M}, —(CH₂)ₘNHS(O)₀₋₂R^{M}, —(CH₂)ₘNO₂, —(CH₂)ₘCN, —(CH₂)ₘR^{P}, heterocycle, aryl, polycyclic heterocyclyl, and heteroaryl, said heterocycle, aryl polycyclic heterocyclyl, and heteroaryl in the definition of R³ are each optionally substituted with halogen, hydroxy, cyano, C₁-C₆ alkyl, C₁-C₆ haloalkyl, C₁-C₆ alkoxy, —C(═O)OR^{M}, or —NR^{J}R^{K}, each R⁴ is separately selected from the group consisting of cyano, an optionally substituted C₁-C₆ alkyl, and an optionally substituted C₁-C₆ heteroalkyl, each R⁵ is separately selected from the group consisting of —(CH₂)ₘOH, —NR^{J}R^{K}, and —(CH₂)ₘC(═O)R^{L}, each R^{L} is independently selected from the group consisting of —OH, C₁-C₆ alkyl, and C₁-C₆ alkoxy, and each R⁶ is separately selected from the group consisting of —NR^{L}C(═O)R^{M}, —NR^{L}C(═O)OR^{M}, —NR^{L}C(═O)NR^{N}R^{O}, —NR^{N}R^{O}, —(CH₂)ₘR^{P}, heterocycle, aryl, polycyclic heterocyclyl, and heteroaryl, said heterocycle, aryl, polycyclic heterocyclyl, and heteroaryl in the definition of R⁶ are each optionally substituted with halogen, cyano, C₁-C₆ alkyl, C₁-C₆ haloalkyl, C₁-C₆ alkoxy, —C(═O)OR^{M}, or —NR^{J}R^{K}.

Some embodiments disclosed herein provide a method of treating a patient, comprising administering to the patient a therapeutically effective amount of a compound having Formula X:

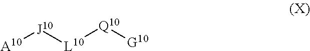

and pharmaceutically acceptable salts, esters, stereoisomers, tautomers or prodrugs thereof;

wherein:

A¹⁰ is selected from the group consisting of C₁-C₆ alkyl, C₁-C₆ alkoxy, C₂-C₆ alkenyl, C₂-C₆ alkynyl, C₃-C₇ cycloalkyl, C₃-C₇ cycloalkenyl, C₁-C₆ haloalkyl, C₁-C₆ heteroalkyl, heterocycle, aryl, and heteroaryl, each optionally substituted with one or more substituents selected from the group consisting of R¹, R², and R³, said aryl and heteroaryl in the definition of A¹⁰ are each further optionally fused with an optionally substituted nonaromatic heterocycle or an optionally substituted nonaromatic carbocycle;

G¹⁰ is selected from the group consisting of C₁-C₆ alkyl, C₁-C₆ alkoxy, C₂-C₆ alkenyl, C₂-C₆ alkynyl, C₃-C₇ cycloalkyl, C₃-C₇ cycloalkenyl, C₁-C₆ haloalkyl, C₁-C₆ heteroalkyl, heterocycle, aryl, and heteroaryl, each optionally substituted with one or more substituents selected from the group consisting of R⁴, R⁵, and R⁶, said aryl and heteroaryl in the definition of G¹⁰ are each further optionally fused with an optionally substituted nonaromatic heterocycle or an optionally substituted nonaromatic carbocycle;

J¹⁰ is a 1-8 atom long spacer containing at least 2 heteroatoms separated by 2 bonds and comprising one or more groups selected from —S(O)₂NR^{A}—, an optionally substituted C₁-C₆ heteroalkyl, an optionally substituted heterocycle, and an optionally substituted heteroalkylheterocycle; including the proviso that J¹⁰ is not a 1-8 atom spacer containing at least 2 heteroatoms separated by 3 or 4 bonds and comprising one or more groups selected from an optionally substituted C₁-C₆ heteroalkyl, an optionally substituted heterocycle, and an optionally substituted heteroalkylheterocycle;

Q¹⁰ is a 1-8 atom long spacer containing at least 2 heteroatoms separated by 2 bonds and comprising one or more groups selected from —S(O)₂NR^{A}—, an optionally substituted C₁-C₆ heteroalkyl, an optionally substituted heterocycle, and an optionally substituted heteroalkylheterocycle;

L¹⁰ is a 2-14 atom long linker comprising one or more groups selected from —O— (oxygen), —C(═O)—, —C(═S)—, —NR^{A}—, —S(O)₀₋₂—, —NR^{A}S(O)₁₋₂NR^{A}—, and —NR^{A}S(O)₀₋₂O—, and one or more groups selected from —O— (oxygen), —C(═O)—, —C(═S)—, —NR^{A}—, —S(O)₀₋₂—, —NR^{A}S(O)₁₋₂NR^{A}—, and —NR^{A}S(O)₂O—, an optionally substituted C₁-C₁₀ alkyl, an optionally substituted aryl, and an optionally substituted heteroaryl; where the an optionally substituted aryl and an optionally substituted heteroaryl in the definition of L¹⁰ are each further optionally fused with an optionally substituted nonaromatic heterocycle or an optionally substituted nonaromatic carbocycle;

R¹ is selected from the group consisting of halogen, optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, optionally substituted C₃-C₇ cycloalkenyl, and an optionally substituted C₁-C₆ heteroalkyl;

R² is selected from the group consisting of halogen, —OR^{A}, —NR^{B}R^{C}, —SR^{A};

R³ is selected from the group consisting of —OR^{D}, —NR^{E}R^{F}, —S(O)₀₋₂R^{D}, —NO₂, —CN, and —(CH₂)ₘR^{G};

R⁴ is selected from the group consisting of halogen, optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, optionally substituted C₃-C₇ cycloalkenyl, an optionally substituted C₁-C₆ heteroalkyl;

R⁵ is selected from the group consisting of —OR^{A}, —NR^{B}R^{C}, —SR^{A};

R⁶ is selected from the group consisting of —OR^{D}, —NR^{E}R^{F}, —S(O)₀₋₂R^{D}, —NO₂, —CN, and —(CH₂)ₘR^{G};

each R^{A} is separately selected from the group consisting of hydrogen, —SO₂R^{F}, —C(═O)R^{F}, —C(═O)NR^{C}R^{D}, C₁-C₆ alkyl, C₂-C₆ alkenyl, C₂-C₆ alkynyl, C₃-C₇ cycloalkyl, C₃-C₇ cycloalkenyl, C₁-C₆ haloalkyl, C₁-C₆ heteroalkyl, and C₁-C₆ heterohaloalkyl, where the C₁-C₆ alkyl, C₃-C₇ cycloalkyl, C₁-C₆ heteroalkyl, and C₁-C₆ heterohaloalkyl in the definition of R^{A} are optionally substituted;

each —NR^{B}R^{C} is separately selected, wherein R^{B} and R^{C} are each independently selected from the group consisting of hydrogen, —SO₂R^{H}, —C(═O)R^{H}, —C(═O)NR^{E}R^{F}, C₁-C₆ alkyl, C₂-C₄ alkenyl, an optionally substituted C₂-C₄ alkynyl, C₃-C₇ cycloalkyl, C₁-C₆ haloalkyl, C₁-C₆ heteroalkyl, heterocycle, and C₁-C₆ heterohaloalkyl where the cycloalkyl and the heterocycle are optionally fused with an aryl or heteroaryl; or —NR^{B}R^{C} is an optionally substituted non-aromatic heterocycle linked through a ring nitrogen atom; or —NR^{B}R^{C} is an optionally substituted C₁-C₆ alkylideneamino;

each R^{D} is independently selected from the group consisting of hydrogen, an optionally substituted C₁-C₆ alkyl, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl, an optionally substituted heterocyle, and —(CH₂)ₘR^{G};

each —NR^{E}R^{F} is separately selected, wherein R^{E} and R^{F} are each independently selected from the group consisting of hydrogen, an optionally substituted C₁-C₆ alkyl, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl, an optionally substituted heterocycle, and —(CH₂)ₘR^{G}; or —NR^{E}R^{F} is an optionally substituted non-aromatic heterocycle linked through a ring nitrogen atom;

each R^{G} is separately selected from an optionally substituted aryl and an optionally substituted heteroaryl;

each R^{H} is separately selected from the group consisting of hydrogen, a C₁-C₆ alkyl, a C₁-C₆ haloalkyl, a C₁-C₆ heteroalkyl, a C₃-C₆ cycloalkyl, an optionally substituted heterocycle, and an optionally substituted aryl or an optionally substituted heteroaryl; and

each m is independently 0, 1, or 2.

Some embodiments disclosed herein provide a method of treating a patient, comprising administering to the patient a therapeutically effective amount of a compound having Formula X having the structure of Formula Xa:

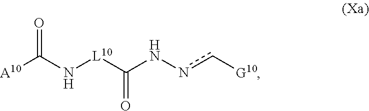

and pharmaceutically acceptable salts thereof, wherein A¹⁰ can be selected from the group consisting of aryl and heteroaryl, each optionally substituted with one or more substituents selected from the group consisting of R¹, R², and R³, said aryl and heteroaryl in the definition of A¹⁰ can each be further optionally fused with a nonaromatic heterocycle or nonaromatic carbocycle; and G¹⁰ can be selected from the group consisting of aryl and heteroaryl, each optionally substituted with one or more substituents selected from the group consisting of R⁴, R⁵, and R⁶, said aryl and heteroaryl in the definition of G¹⁰ can each be further optionally fused with a nonaromatic heterocycle or nonaromatic carbocycle.

Some embodiments disclosed herein provide a method of treating a patient, comprising administering to the patient a therapeutically effective amount of a compound having Formula X having the structure of Formula Xb:

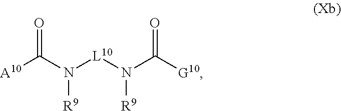

and pharmaceutically acceptable salts thereof, wherein A¹⁰ can be selected from the group consisting of aryl and heteroaryl, each optionally substituted with one or more substituents selected from the group consisting of R¹, R², and R³, said aryl and heteroaryl in the definition of A¹⁰ can each be further optionally fused with a nonaromatic heterocycle or nonaromatic carbocycle; and G¹⁰ can be selected from the group consisting of aryl and heteroaryl, each optionally substituted with one or more substituents selected from the group consisting of R⁴, R⁵, and R⁶, said aryl and heteroaryl in the definition of G¹⁰ can each be further optionally fused with a nonaromatic heterocycle or nonaromatic carbocycle.

Some embodiments disclosed herein provide a method of treating a patient, comprising administering to the patient a therapeutically effective amount of a compound having Formula X, wherein A¹⁰ can be selected from the group consisting of C₁-C₆ alkyl, C₃-C₇ cycloalkyl, C₁-C₆ heteroalkyl, heterocycle, aryl, and heteroaryl, each substituted with one or more substituents selected from the group consisting of R¹, R², and R³, said aryl and heteroaryl in the definition of A¹⁰ can each be further optionally fused with a nonaromatic heterocycle or nonaromatic carbocycle; G¹⁰ can be selected from the group consisting of C₁-C₆ alkyl, C₃-C₇ cycloalkyl, C₁-C₆ heteroalkyl, heterocycle, aryl, and heteroaryl, each substituted with one or more substituents selected from the group consisting of R⁴, R⁵, and R⁶, said aryl and heteroaryl in the definition of G¹⁰ can each be further optionally fused with a nonaromatic heterocycle or nonaromatic carbocycle; R¹ can be selected from the group consisting of fluorine, chlorine, and methyl; R² can be selected from the group consisting of —OR^{A}, —NR^{B}R^{C}, and —SR^{A}; R³ can be selected from the group consisting of —(CH₂)ₘR^{G}, —OR^{D}, and —NR^{E}R^{F}; R⁴ can be selected from the group consisting of fluorine, chlorine, and methyl; R⁵ can be selected from the group consisting of —OR^{A}, —NR^{B}R^{C}, and —SR^{A}; R⁶ can be selected from the group consisting of —(CH₂)ₘR^{G}, —OR^{D}, and —NR^{E}R^{F}; J¹⁰ and Q¹⁰ can each be independently selected from the group consisting of an ester, an amide, a urea, a carbamide, —S(O)₂NR^{A}—, a thioamide, a thioester, and an imidamide; L¹⁰ can be a 3-13 atom long linker comprising comprising one or more groups selected from —O— (oxygen), —NR^{A}—, —S(O)₀₋₂—, and —NR^{A}S(O)₁₋₂O—, and one or more groups selected from —O— (oxygen), —NR^{A}—, —S(O)₀₋₂—, and —NR^{A}S(O)₁₋₂O—, an optionally substituted C₁-C₈ alkyl, an optionally substituted aryl, and an optionally substituted heteroaryl; where the an optionally substituted aryl and an optionally substituted heteroaryl in the definition of L¹⁰ can each be further optionally fused with a nonaromatic heterocycle or a nonaromatic carbocycle; and each m can be independently 0, 1, or 2.

Some embodiments disclosed herein provide a method of treating a patient, comprising administering to the patient a therapeutically effective amount of a compound having Formula X, wherein A¹⁰ can be selected from the group consisting of a C₂-C₆ alkyl, a C₂-C₇ cycloalkyl, a C₁-C₆ heteroalkyl, a heterocycle, phenyl, pyridinyl, pyrrolyl, pyrimidinyl, imidazolyl, isoxazolyl, thiazolyl, thienyl, indolyl, benzoxazolyl, benzthiazolyl, benzimidazolyl, and purinyl, each substituted with one or more substituents selected from the group consisting of R¹, R², and R³; G¹⁰ can be selected from the group consisting of a C₂-C₆ alkyl, a C₂-C₇ cycloalkyl, a C₁-C₆ heteroalkyl, a heterocycle, phenyl, pyridinyl, pyrrolyl, pyrimidinyl, imidazolyl, isoxazolyl, thiazolyl, thienyl, indolyl, benzoxazolyl, benzthiazolyl, benzimidazolyl, and purinyl, each substituted with one or more substituents selected from the group consisting of R⁴, R⁵, and R⁶; R¹ can be selected from the group consisting of fluorine, chlorine, and methyl; R² can be selected from the group consisting of —OR^{A} and —NR^{B}R^{C}; R³ can be R^{G}; R⁴ can be selected from the group consisting of fluorine, chlorine, and methyl; R⁵ can be selected from the group consisting of —OR^{A} and —NR^{A}R^{B}; R⁶ can be R^{G}; J¹⁰ and Q¹⁰ can each be independently selected from the group consisting of an amide, a urea, a carbamide, —S(O)₂NR^{A}—, a thioamide, and an imidamide; L¹⁰ can be a 3-13 atom long linker comprising one or more groups selected from —O— (oxygen), —C(═O)—, —NR^{A}—, —S(O)₀₋₂—, —NR^{A}S(O)₁₋₂NR^{A}—, and one or more groups selected from an optionally substituted aryl, and an optionally substituted heteroaryl; where the an optionally substituted aryl and an optionally substituted heteroaryl in the definition of L¹⁰ can each be further optionally fused with a nonaromatic heterocycle or a nonaromatic carbocycle.

In some specific embodiments disclosed herein provide a method of treating a patient, comprising administering to the patient a therapeutically effective amount of a compound having the structure:

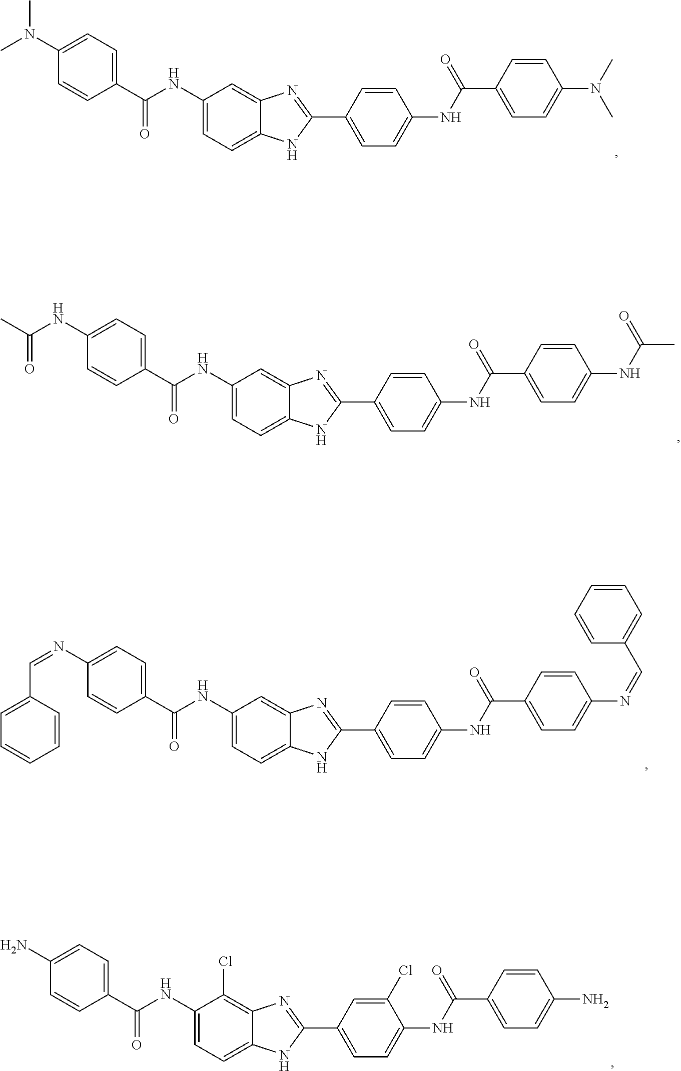

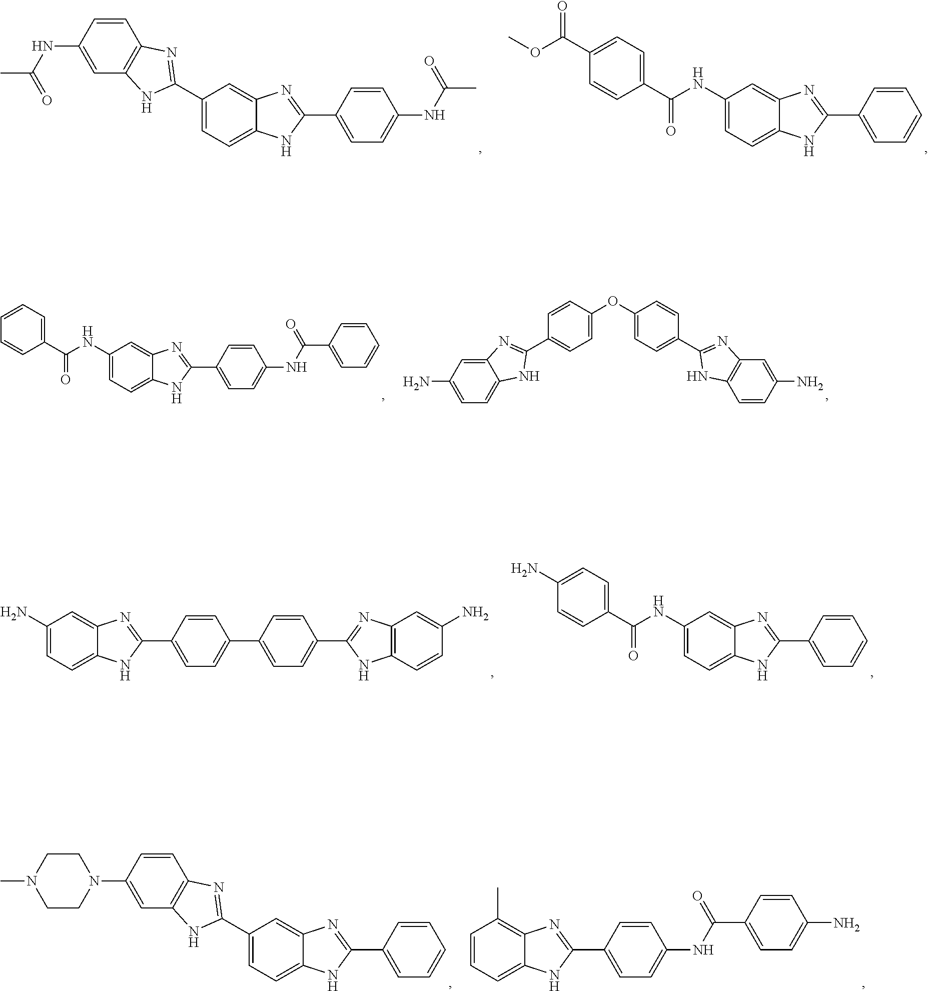

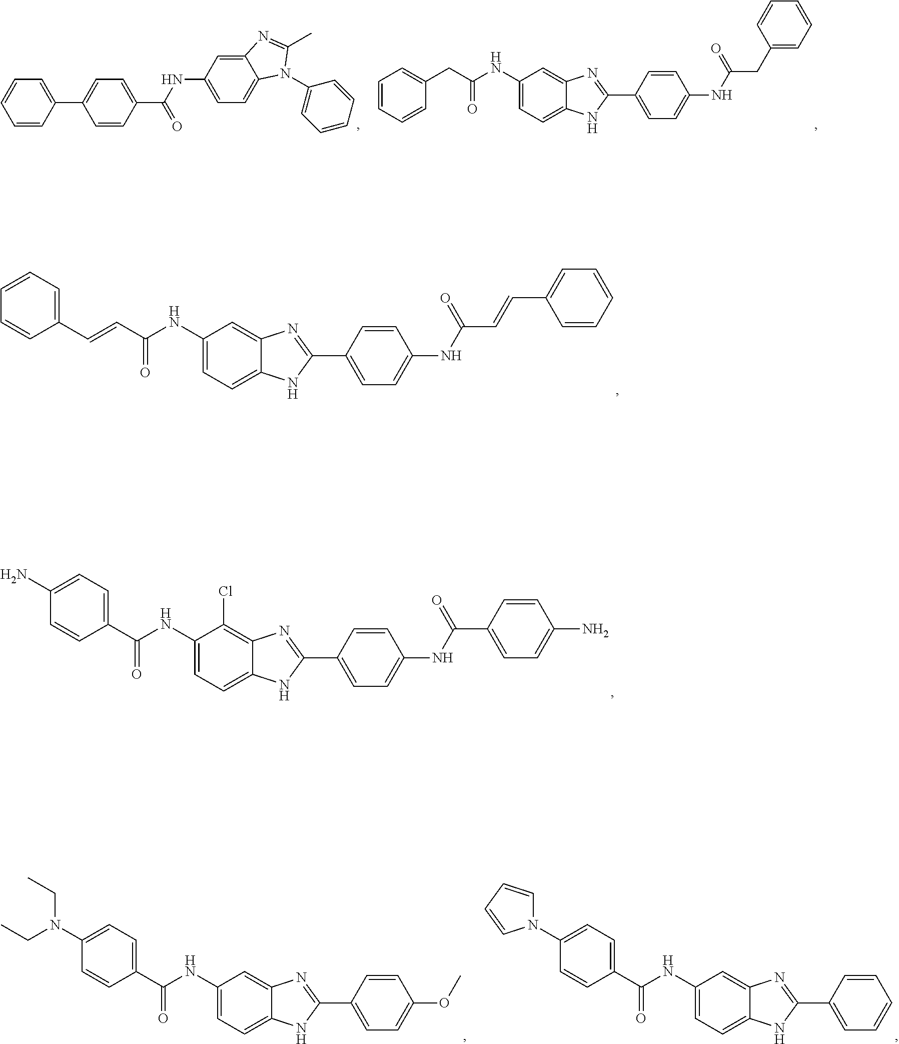

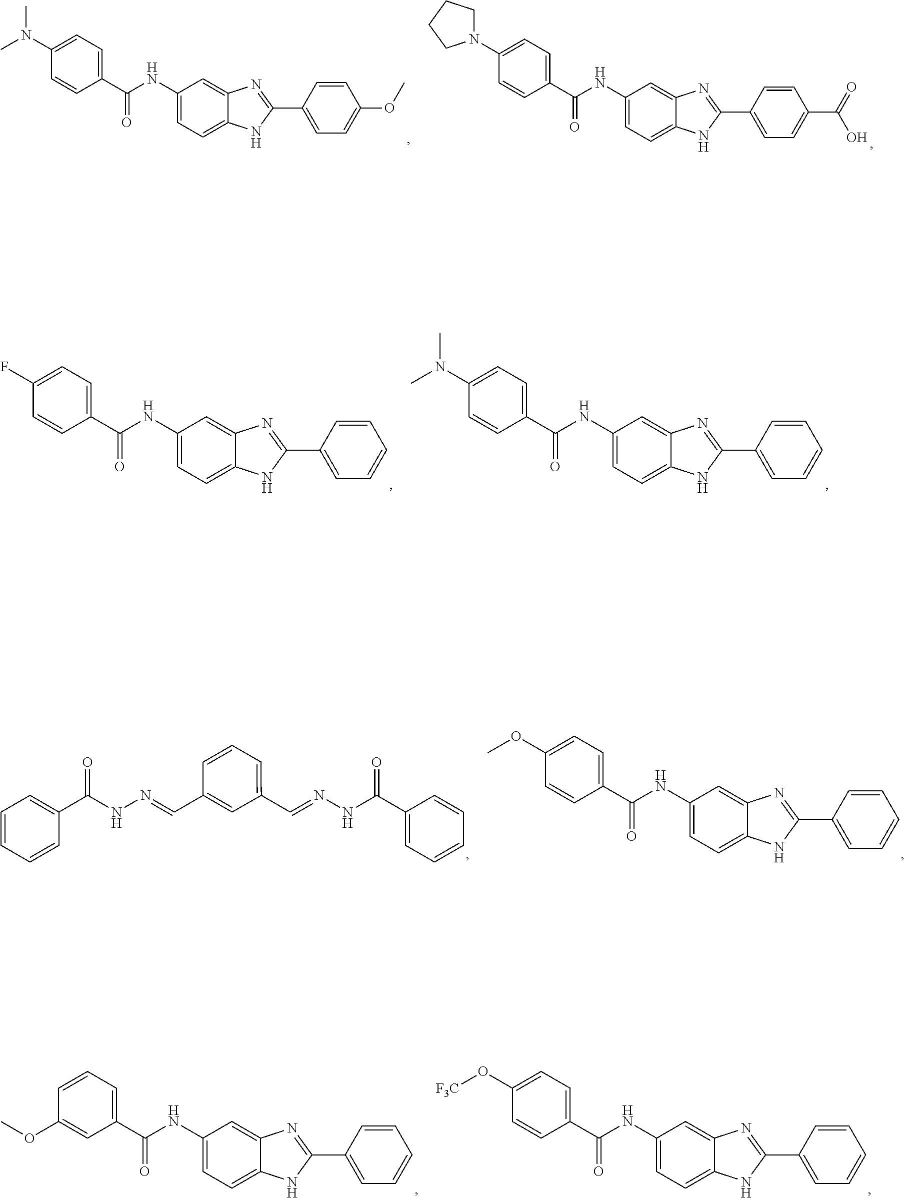

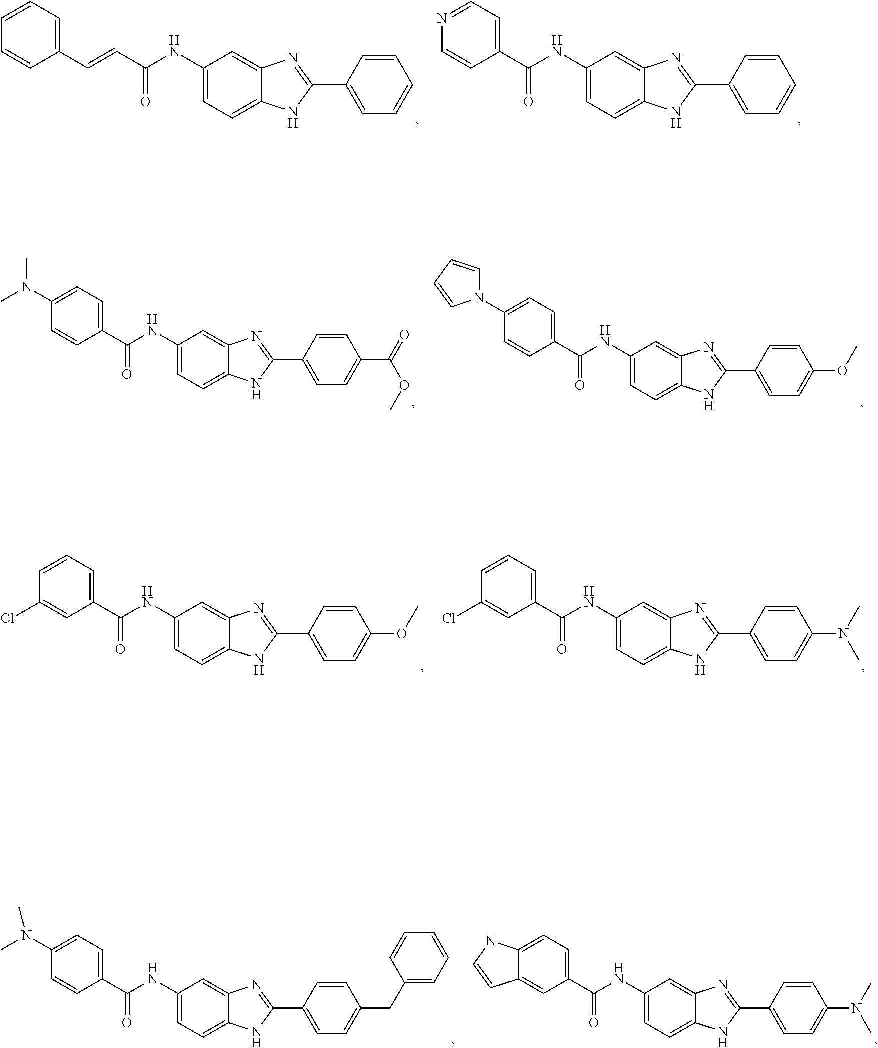

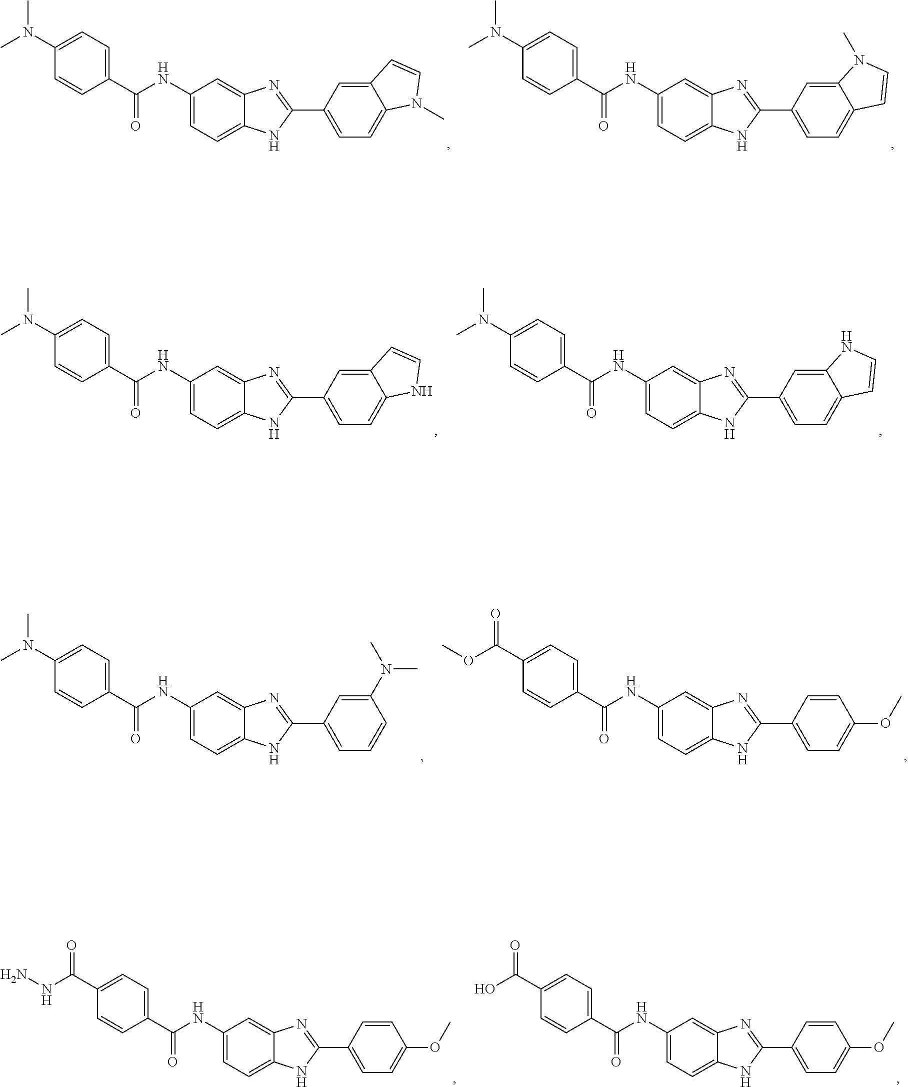

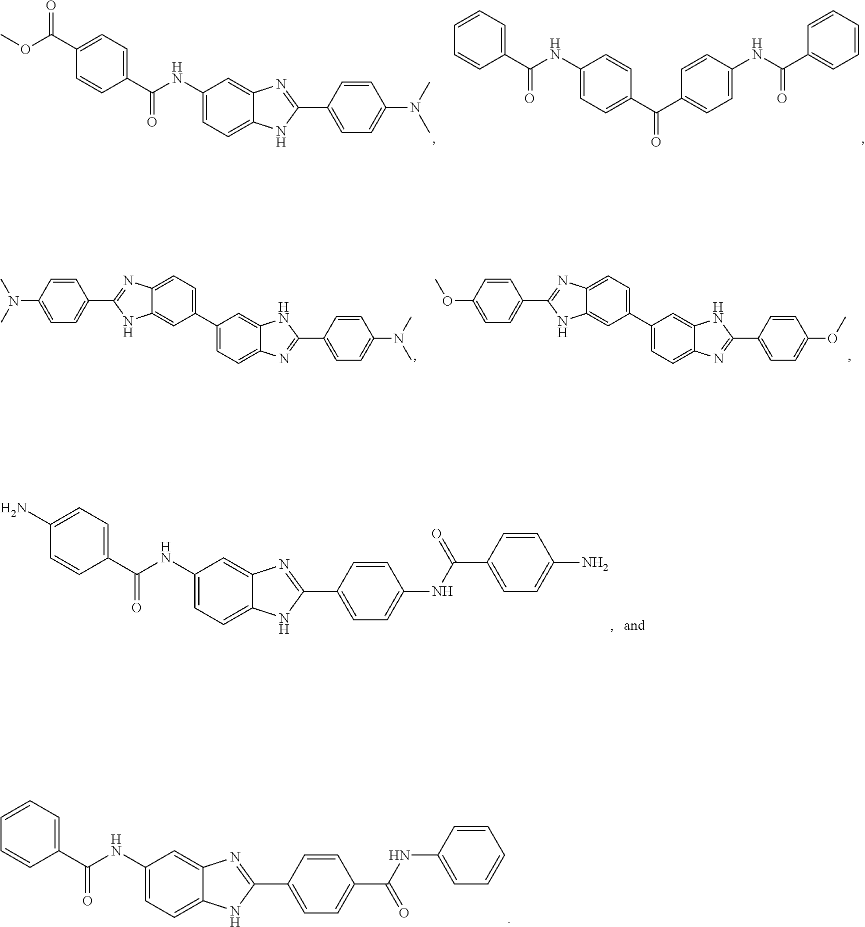

### Certain Synthesis Methods

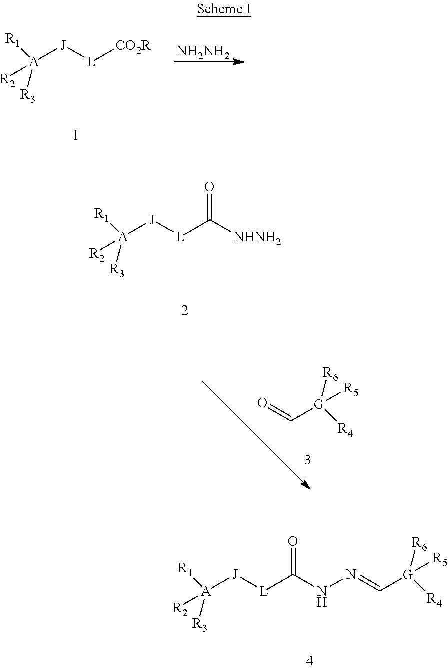

The process of Scheme I describes the general synthesis of compounds of general structure 4 described in Formula I, wherein R can be C₁₋₆alkyl, aryl and the like. Treatment of the ester derivatives of general structure 1 with hydrazine affords hydrazide derivatives of general structure 2. The intermediates of general structure 2 are condensed with an aldehyde of general structure 3 to generate the compounds of general structure 4.

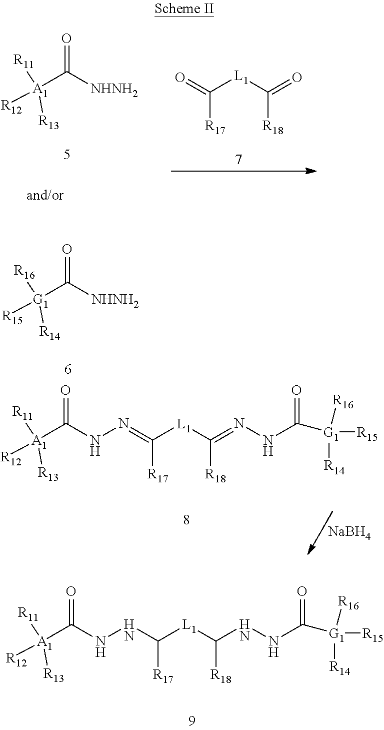

The process of Scheme II describes general synthesis of the compounds of Formula II. Condensation reactions of the biscarbonyl compounds of general structure 7 and substituted hydrazides of general structures 5 or 6 under standard conditions provide the pseudo-symmetric compounds of structure 8. Alternatively, when the hydrazides of general structures 5 and 6 are different, the condensation reactions can be run sequentially to provide compounds of general structure 8. Compounds of general structure 9 can be obtained by a standard reduction of compounds of general structure 8. For example, reducing agents such sodium borohydride, lithium borohydride, sodium cyanoborohydride, potassium trisiamylborohydride, potassium tri-sec-butylborohydride, lithium trisiamylborohydride, lithium tri-sec-butylborohydride, diisobutylaluminum hydride, lithium triethoxyaluminum hydride and the like can be used in the reduction.

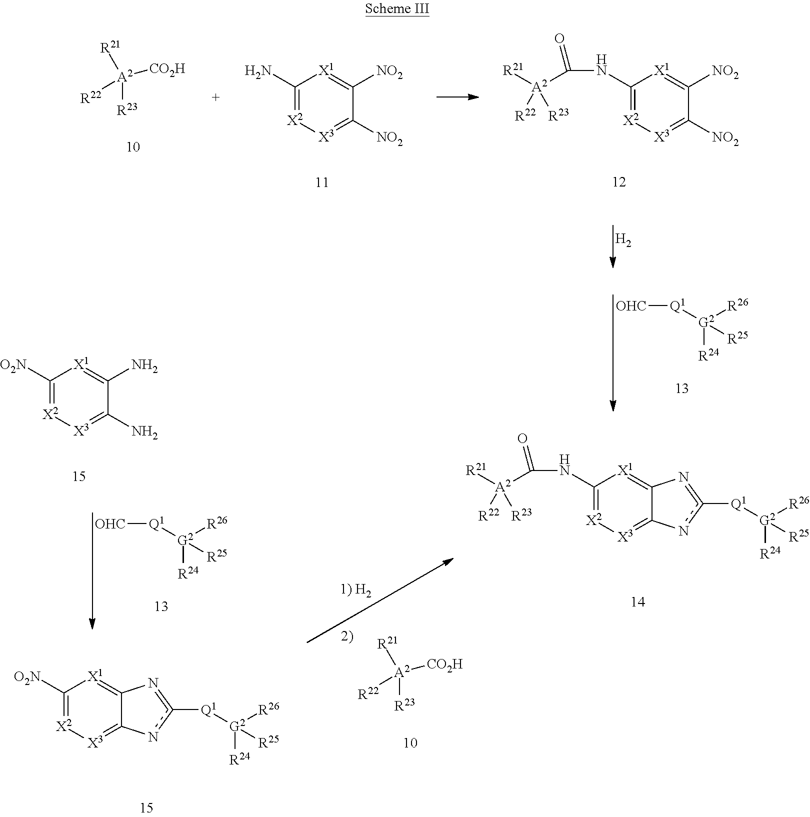

The process of Scheme III describes general synthesis of the compounds of Formula III. Coupling of an acid derivative of structure 10 with an amino derivative of structure 11 under standard conditions provides the amide intermediate of structure 12. The two nitro groups of structure 12 are reduced under a typical reduction condition such as a metal catalyzed hydrogenation to give the diamino intermediate and then the intermediate is condensed with an aldehyde of structure 13 under an oxidative condition to afford desired compounds of structure 14. Alternatively, compounds of structure 10 can be prepared from diamino derivatives of structure 15. Condensation reaction of compounds of structure 15 with compounds of structure 13 under an oxidative condition provides the intermediate of structure 16. A nitro reduction of intermediates 16 followed by an amide formation reaction under similar conditions described previously generate the compounds of structure 14.

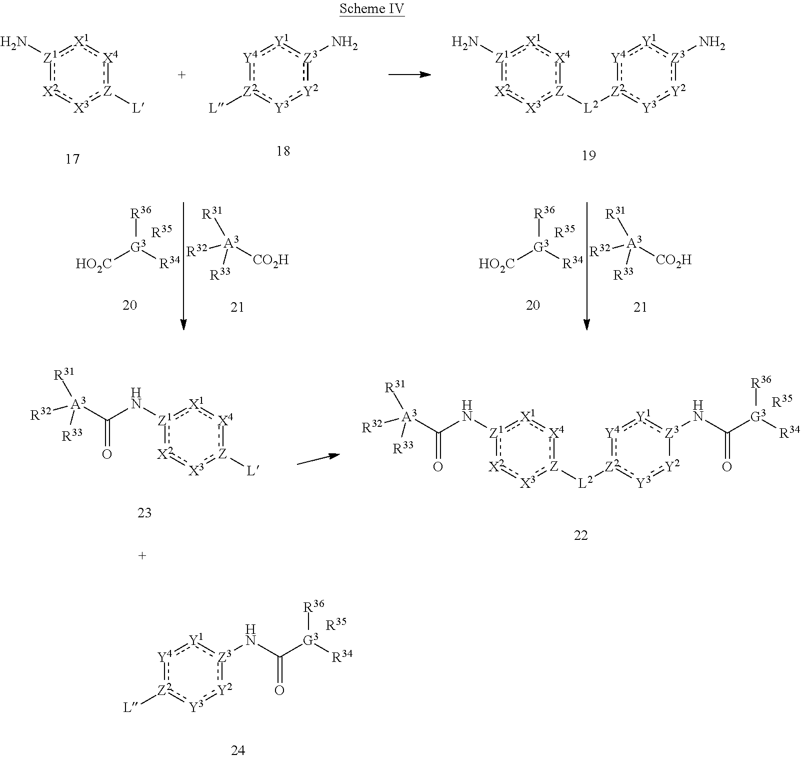

The process of Scheme IV describes general synthesis of the compounds of Formula IV. Coupling reaction of compounds of structure 17 and compounds of structure 18 under the standard condition based on the nature of substituents L′ and L″ provides intermediates of structure 19. Amide formation reaction of compounds of structures 20 and 21 and intermediates of structure 19 affords the products of structure 22. Alternatively, especially for compounds of structure 22 that have different rings or side chains, compounds of structure 22 can be prepared with different coupling strategy. Amide coupling reactions between compounds of structures 17 and 21 and between compounds of structures 18 and 20 generate separate intermediates of structures 23 and 24. The intermediates of structures 23 and 24 are then coupled to form final compounds of structure 22.

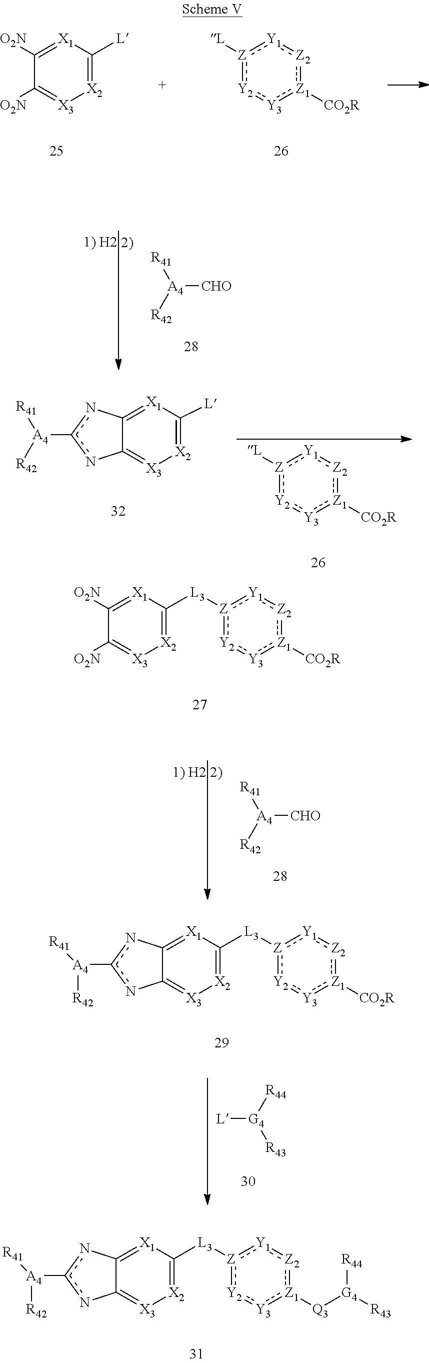

The process of Scheme V describes general synthesis of the compounds of Formula V. General coupling reaction of compounds of structures 25 and 26 affords the intermediates of structure 27. A reduction of intermediate of structure 27 by a reducing agent such as metal catalyzed hydrogenation followed by an oxidative condensation with compounds of structure 28 give intermediates of structure 29. The acid derivatives of structure 29 are coupled with compounds of structure 30 to provide the final products of structure 31. Alternatively, compounds of structure 25 can be reduced and coupled with aldehydes 28 to provide bicyclic imidazole derivatives of structure 32. Coupling reaction between compounds of structures 32 and 26 affords the same intermediates of structure 29.

### EXAMPLES

The following examples are set forth merely to assist in understanding the embodiments and should not be construed as limiting the embodiments described and claimed herein in any way. Variations of the invention, including the substitution of all equivalents now known or later developed, which would be within the purview of those skilled in the art, and changes in formulation or minor changes in experimental design, are to be considered to fall within the scope of the invention incorporated herein.

### Example 1

### (N′,N′″E,N′,N′″E)-N′,N′″-((Oxybis(4,1-phenylene))bis(methanylylidene))bis(4-(dimethylamino)benzohydrazide) (Compound 101)

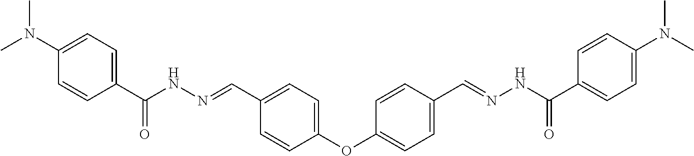

Compound 101 was prepared according to the procedure described in Scheme II. To a solution of 4,4′-oxybisbenzaldehyde (Aldrich, 42 mg, 0.186 mmol) and 4-dimethylaminobenzyhydrazide (Alfa Aesar, 73 mg, 0.408 mmol) in 3 mL of ethanol was added 3 drops of acetic acid. The reaction was heated to 60° C. for 12 h, cooled to room temperature and filtered. The white precipitate was washed with water (5 mL) followed by methanol (5 mL) and dried under vacuum to yield 55 mg of compound 101 as a white powder. [M+H]⁺ calcd for C₃₂H₃₃N₆O₃: 549.26. Found: 549.05.

### Example 2

### (N′,N′″E,N′,N′″E)-N′,N′″-((oxybis(4,1-phenylene))bis(methanylylidene))bis(3-methoxybenzohydrazide) (Compound 102)

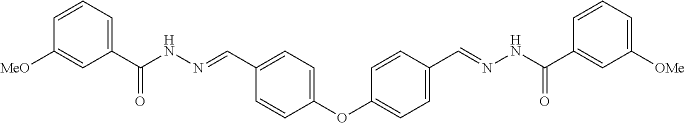 [SMILES: COc1cccc(C(=O)N/N=C/c2ccc(Oc3ccc(/C=N/NC(=O)c4cccc(OC)c4)cc3)cc2)c1]

Compound 102 was prepared according to the procedure described in Scheme II from 4,4′-oxybisbenzaldehyde and 3-methoxybenzoate. [M+H]⁺ calcd for C₃₀H₂₆N₄O₅: 523.20. Found: 513.15.

### Example 3

### (N′,N′″E,N′,N′″E)-N′,N′″-(1,3-phenylenebis(methanylylidene))bis(4-(dimethylamino)benzohydrazide) (Compound 103)

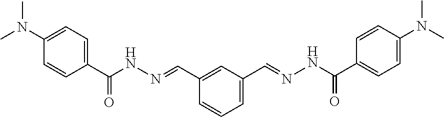

Compound 103 was prepared according to the procedure described in Scheme II from benzene-1,3-dicarboxaldehyde and 4-dimethylaminobenzoate. [M+H]⁺ calcd for C₂₆H₂₈N₆O₂: 457.23. Found: 457.01.

### Example 4

### (N′,N′″E,N′,N′″E)-N′N′″-((5-methoxy-1,3-phenylene)bis(methanylylidene))bis(4-(dimethylamino)benzohydrazide) (Compound 104)

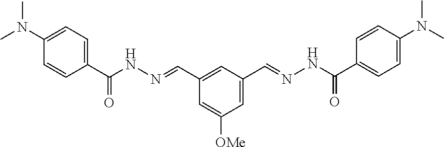

Compound 104 was prepared according to the procedure described in Scheme II from 5-5-methoxybenzene-1,3-dicarboxaldehyde and 4-dimethylaminobenzoate. ¹H NMR (300 MHz, DMSO-d₆) δ 11.62 (s, 2H), 8.41 (s, 2H), 7.81 (d, J=7.5 Hz, 4H), 7.60 (s, 1H), 7.26 (d, J=3 Hz, 2H), 6.76 (d, J=7.5 Hz, 4H), 3.84 (s, 3H), 2.99 (s, 12H).

### Example 5

### (N′,N′″E,N′,N′″E)-N′,N′″-((5-methoxy-1,3-phenylene)bis(methanylylidene))bis(4-methoxybenzohydrazide) (Compound 105)

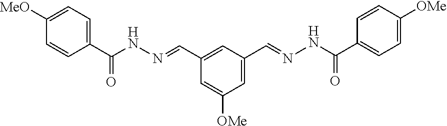 [SMILES: COc1ccc(C(=O)N/N=C/c2cc(/C=N/NC(=O)c3ccc(OC)cc3)cc(OC)c2)cc1]

Compound 105 was prepared according to the procedure described in Scheme II from 5-5-methoxybenzene-1,3-dicarboxaldehyde and 4-methoxybenzoate. [M+H]⁺ calcd for C₂₅H₂₄N₄O₅: 461.18. Found: 461.00.

### Example 6

### (N′,N′″E,N′,N′″E)-N′,N′″-((4-methoxy-1,3-phenylene)bis(methanylylidene))bis(4-(dimethylamino)benzohydrazide) (Compound 106)

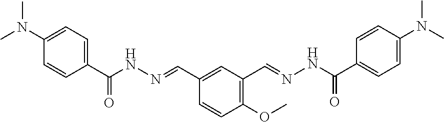

Compound 106 was prepared according to the procedure described in Scheme II from 4-4-methoxybenzene-1,3-dicarboxaldehyde and 4-dimethylaminobenzoate. 1H NMR (500 MHz, DMSO-d₆) δ 11.72 (s, 1H), 11.58 (s, 1H), 8.88 (s, 1H), 8.51 (s, 1H), 8.33 (s, 1H), 7.94 (dd, J=7.5, 15 Hz, 4H), 7.80 (d, J=7.5 Hz, 1H), 7.29 (d, J=7.5 Hz, 1H), 6.86 (d, J=7.5 Hz, 4H), 4.10 (s, 3H), 3.10 (s, 12H).

### Example 7

### (N′,N′″E,N′,N′″E)-N′,N′″-((5-(benzyloxy)-1,3-phenylene)bis(methanylylidene))bis(4-(dimethylamino)benzohydrazide) (Compound 107)

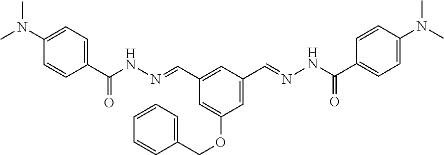

Compound 107 was prepared according to the procedure described in Scheme II from 5-5-benzyloxybenzene-1,3-dicarboxaldehyde and 4-dimethylaminobenzoate. [M+H]⁺ calcd for C₃₃H₃₅N₆O₃: 563.28. Found: 563.08.

### Example 8

### 2(N′,N′″E,N′,N′″E)-N′,N′″-([1,1′-biphenyl]-3,5-diylbis(methanylylidene))bis(4-(dimethylamino)benzohydrazide) (Compound 108)

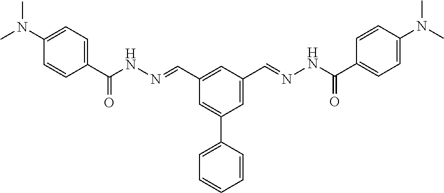

Compound 108 was prepared according to the procedure described in Scheme II from 5-phenylbenzene-1,3-dicarboxaldehyde and 4-dimethylaminobenzoate. [M+H]⁺ calcd for C₃₂H₃₂N₆O₂: 533.26. Found: 533.08.

### Example 9

### (N′,N′″E,N′,N′″E)-N′,N′″-(1,3-phenylenebis(methanylylidene))bis(benzo[d][1,3]dioxole-5-carbohydrazide) (Compound 109)

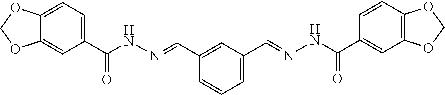 [SMILES: O=C(N/N=C/c1cccc(/C=N/NC(=O)c2ccc3c(c2)OCO3)c1)c1ccc2c(c1)OCO2]

Compound 109 was prepared according to the procedure described in Scheme II from benzene-1,3-dicarboxaldehyde and 5-(1,3-dioxolano)benzoate. ¹H NMR (500 MHz, DMSO-d₆) δ 11.88 (s, 2H), 8.58 (s, 2H), 8.19 (s, 1H), 7.85 (d, J=6.3 Hz, 2H), 7.64 (m, 3H), 7.56 (s, 2H), 7.17 (d, J=6 Hz, 2H), 6.22 (s, 4H).

### Example 10

### (N′,N′″E,N′,N′″E)-N′,N′″-(1,3-phenylenebis(methanylylidene))bis(4-(1H-pyrrol-1-yl)benzohydrazide) (Compound 110)

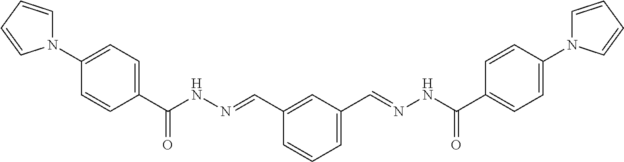 [SMILES: O=C(N/N=C/c1cccc(/C=N/NC(=O)c2ccc(-n3cccc3)cc2)c1)c1ccc(-n2cccc2)cc1]

Compound 110 was prepared according to the procedure described in Scheme II from benzene-1,3-dicarboxaldehyde and 4-pyrrolebenzoate. ¹H NMR (500 MHz, DMSO-d₆) δ 12.15 (s, 2H), 8.63 (s, 2H), 8.24 (s, 1H), 8.13 (d, J=7.5 Hz, 4H), 7.89 (d, J=7.5 Hz, 6H), 7.64 (m, 5H), 6.43 (s, 4H).

### Example 11

### (N′,N′″E,N′,N′″E)-N′,N′″-((5-(dimethylamino)-1,3-phenylene)bis(methanylylidene))bis(4-(dimethylamino)benzohydrazide) (Compound 111)

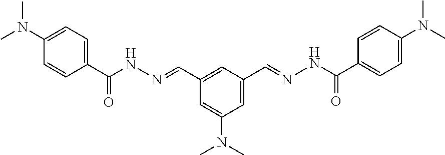

Compound 111 was prepared according to the procedure described in Scheme II from 5-dimethylaminobenzene-1,3-dicarboxaldehyde and 4-dimethylaminobenzoate. [M+H]⁺ calcd for C₂₈H₃₃N₇O₂: 500.27. Found: 500.07.

### Example 12

### (N′,N′″E,N′″E)-N′,N′″-(1,3-phenylenebis(methanylylidene))bis(4-isopropylbenzohydrazide) (Compound 112)

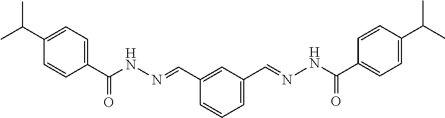 [SMILES: CC(C)c1ccc(C(=O)N/N=C/c2cccc(/C=N/NC(=O)c3ccc(C(C)C)cc3)c2)cc1]

Compound 112 was prepared according to the procedure described in Scheme II from benzene-1,3-dicarboxaldehyde and 4-isopropylbenzoate. ¹H NMR (500 MHz, DMSO-d₆) δ 11.9 (s, 2H), 8.51 (s, 2H), 8.12 (s, 1H), 7.87 (d, J=8.1 Hz, 4H), 7.77 (d, J=7.3 Hz, 2H), 7.56 (dd, J=7.3, 7.3 Hz, 1H), 7.42 (d, J=8.1 Hz, 4H), 3.01-2.96 (m, 2H), 1.25 (d, J=6.8 Hz, 12H).

### Example 13

### (N′,N′″E,N′,N′″E)-N′,N′″-((5-(allyloxy)-1,3-phenylene)bis(methanylylidene))bis(4-(dimethylamino)benzohydrazide) (Compound 113)

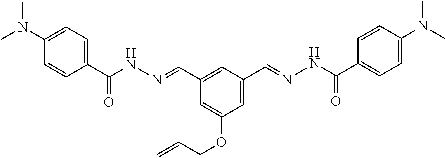

Compound 113 was prepared according to the procedure described in Scheme II from 5-allyloxybenzene-1,3-dicarboxaldehyde and 4-dimethylaminobenzoate. [M+H]⁺ calcd for C₂₉H₃₂N₆O₃: 513.26. Found: 513.10.

### Example 14

### (N′,N′″E,N′,N′″E)-N′,N′″-((5-(benzylamino)-1,3-phenylene)bis(methanylylidene))bis(4-(dimethylamino)benzohydrazide) (Compound 114)

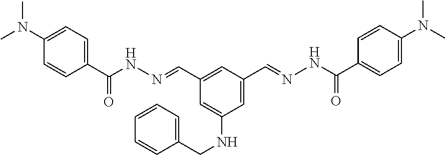

Compound 114 was prepared according to the procedure described in Scheme II from 5-benzylaminobenzene-1,3-dicarboxaldehyde and 4-dimethylaminobenzoate. [M+H]⁺ calcd for C₃₃H₃₅N₇O₂: 562.29. Found: 562.17.

### Example 15

### (N′,N′″E,N′″E)-N′,N′″-(1,3-phenylenebis(methanylylidene))bis(4-(bis(2,2,2-trifluoroethyl)amino)benzohydrazide) (Compound 115)

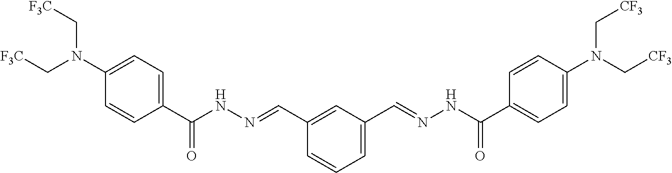 [SMILES: O=C(N/N=C/c1cccc(/C=N/NC(=O)c2ccc(N(CC(F)(F)F)CC(F)(F)F)cc2)c1)c1ccc(N(CC(F)(F)F)CC(F)(F)F)cc1]

Compound 115 was prepared according to the procedure described in Scheme II from benzene-1,3-dicarboxaldehyde and 4-di(2,2,2-trifluoroethyl)aminobenzoate. ¹H NMR (500 MHz, DMSO-d₆) δ 11.7 (s, 2H), 8.47 (s, 2H), 8.08 (s, 1H), 7.83 (d, J=8.3 Hz, 4H), 7.72 (m, 2H), 7.53 (t, J=7.8 Hz, 1H), 7.19 (d, J=7.8 Hz, 4H), 4.49 (q, J_{H-F}=8.3 Hz, 8H).

### Example 16

### N-(3,5-bis((E)-(2-(4-(dimethylamino)benzoyl)hydrazono)methyl)phenyl)benzamide

(Compound 116)

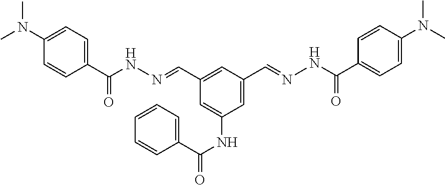

Compound 116 was prepared according to the procedure described in Scheme II from 5-benzoylaminobenzene-1,3-dicarboxaldehyde and 4-dimethylaminobenzoate. [M+H]⁺ calcd for C₃₅H₃₈N₈O₃: 619.31. Found: 619.16.

### Example 17

### (N′,N′″E,N′″E)-N′,N′″-(1,3-phenylenebis(methanylylidene))bis(4-(isopropylamino)benzohydrazide) (Compound 117)

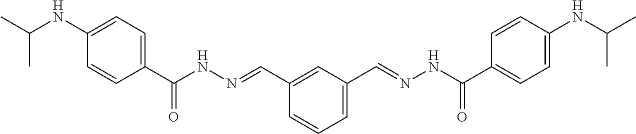 [SMILES: CC(C)Nc1ccc(C(=O)N/N=C/c2cccc(/C=N/NC(=O)c3ccc(NC(C)C)cc3)c2)cc1]

Compound 117 was prepared according to the procedure described in Scheme II from benzene-1,3-dicarboxaldehyde and 4-isopropylaminobenzoate. ¹H NMR (500 MHz, DMSO-d₆) 11.52 (s, 2H), 8.45 (s, 2H), 8.04 (s, 1H), 7.74-7.69 (m, 6H), 7.52 (t, J=7.8 Hz, 1H), 6.61 (d, J=8.8 Hz, 4H), 6.16 (d, J=7.8 Hz, 2H), 3.68-3.61 (m, 2H), 1.16 (d, J=6.3 Hz, 12H).

### Example 18

### (N′,N′″E,N′″E)-N′,N′″-(1,3-phenylenebis(methanylylidene))bis(4-(cyclopentylamino)benzohydrazide) (Compound 118)

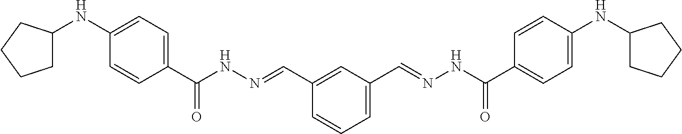 [SMILES: O=C(N/N=C/c1cccc(/C=N/NC(=O)c2ccc(NC3CCCC3)cc2)c1)c1ccc(NC2CCCC2)cc1]

Compound 118 was prepared according to the procedure described in Scheme II from benzene-1,3-dicarboxaldehyde and 4-cyclopentylaminobenzoate. ¹H NMR (500 MHz, DMSO-d₆) δ 11.95 (s, 1H), 7.91 (d, J=8.9 Hz, 1H), 7.17 (d, J=8.9 Hz, 1H), 6.70 (s, 1H), 5.28 (q, J=9.2 Hz, 2H), 3.04 (q, J=7.3 Hz, 2H), 2.44 (s, 3H), 1.09 (t, J=7.3 Hz, 3H).

### Example 19

### (N′,N′″E,N′,N′″E)-N′,N′″-(1,3-phenylenebis(methanylylidene))bis(([1,1′-biphenyl]-4-carbohydrazide)) (Compound 119)

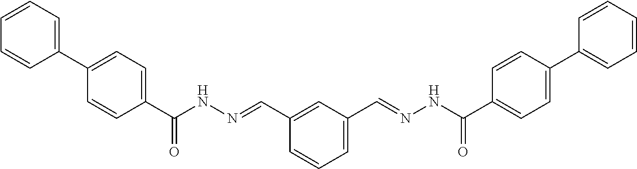 [SMILES: O=C(N/N=C/c1cccc(/C=N/NC(=O)c2ccc(-c3ccccc3)cc2)c1)c1ccc(-c2ccccc2)cc1]

Compound 119 was prepared according to the procedure described in Scheme II from benzene-1,3-dicarboxaldehyde and 4-phenylbenzoate. ¹H NMR (500 MHz, DMSO-d₆) δ 11.95 (s, 1H), 7.91 (d, J=8.9 Hz, 1H), 7.17 (d, J=8.9 Hz, 1H), 6.70 (s, 1H), 5.28 (q, J=9.2 Hz, 2H), 3.04 (q, J=7.3 Hz, 2H), 2.44 (s, 3H), 1.09 (t, J=7.3 Hz, 3H).

### Example 20

### (N′,N′″E,N′″E)-N′,N′″-(1,3-phenylenebis(methanylylidene))bis(4-(diethylamino)benzohydrazide) (Compound 120)

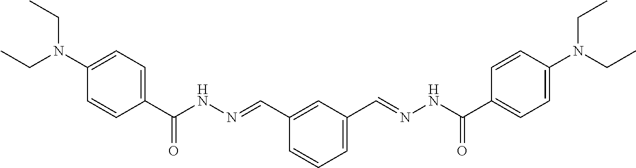 [SMILES: CCN(CC)c1ccc(C(=O)N/N=C/c2cccc(/C=N/NC(=O)c3ccc(N(CC)CC)cc3)c2)cc1]

Compound 120 was prepared according to the procedure described in Scheme II from benzene-1,3-dicarboxaldehyde and 4-diethylaminobenzoate. ¹H NMR (500 MHz, DMSO-d₆) δ 11.95 (s, 1H), 7.91 (d, J=8.9 Hz, 1H), 7.17 (d, J=8.9 Hz, 1H), 6.70 (s, 1H), 5.28 (q, J=9.2 Hz, 2H), 3.04 (q, J=7.3 Hz, 2H), 2.44 (s, 3H), 1.09 (t, J=7.3 Hz, 3H).

### Example 21

### (N′,N′″E,N′″E)-N′,N′″-(1,3-phenylenebis(methanylylidene))bis(2-naphthohydrazide) (Compound 121)

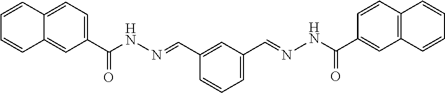 [SMILES: O=C(N/N=C/c1cccc(/C=N/NC(=O)c2ccc3ccccc3c2)c1)c1ccc2ccccc2c1]

Compound 121 was prepared according to the procedure described in Scheme II from benzene-1,3-dicarboxaldehyde and 4-dimethylaminobenzoate. ¹H NMR (500 MHz, DMSO-d₆) δ 11.95 (s, 1H), 7.91 (d, J=8.9 Hz, 1H), 7.17 (d, J=8.9 Hz, 1H), 6.70 (s, 1H), 5.28 (q, J=9.2 Hz, 2H), 3.04 (q, J=7.3 Hz, 2H), 2.44 (s, 3H), 1.09 (t, J=7.3 Hz, 3H).

### Example 22

### (N′,N′″E,N′″E)-N′,N′″-(1,3-phenylenebis(methanylylidene))bis(4-(1H-imidazol-1-yl)benzohydrazide) (Compound 122)

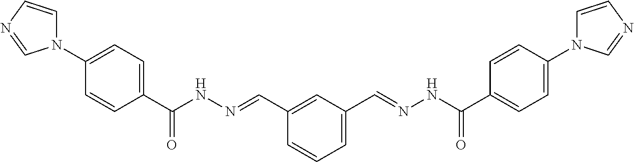 [SMILES: O=C(N/N=C/c1cccc(/C=N/NC(=O)c2ccc(-n3ccnc3)cc2)c1)c1ccc(-n2ccnc2)cc1]

Compound 122 was prepared according to the procedure described in Scheme II from benzene-1,3-dicarboxaldehyde and 4-imidazolebenzoate. ¹H NMR (500 MHz, DMSO-d₆) δ 12.0 (s, 2H), 8.55 (s, 2H), 8.49 (s, 2H), 8.16 (s, 1H), 8.09 (d, J=8.3 Hz, 4H), 7.93 (s, 2H), 7.89 (d, J=7.5 Hz, 4H), 7.81 (d, J=7.5 Hz, 2H), 7.58 (t, J=7.6 Hz, 1H), 7.21 (bs, 2H).

### Example 23

### (N′,N′″E,N′″E)-N′,N′″-(1,3-phenylenebis(methanylylidene))bis(4-((4-fluorobenzyl)amino)benzohydrazide) (Compound 123)

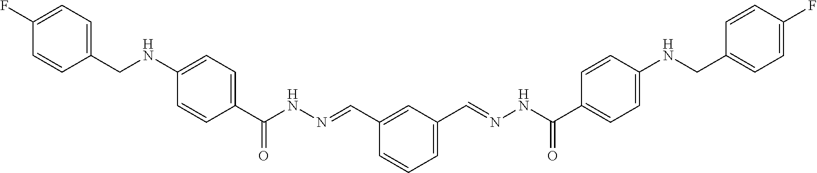 [SMILES: O=C(N/N=C/c1cccc(/C=N/NC(=O)c2ccc(NCc3ccc(F)cc3)cc2)c1)c1ccc(NCc2ccc(F)cc2)cc1]

Compound 123 was prepared according to the procedure described in Scheme II from benzene-1,3-dicarboxaldehyde and 4-(4-fluorobenzylamino)benzoate. ¹H NMR (500 MHz, DMSO-d₆) δ 11.95 (s, 1H), 7.91 (d, J=8.9 Hz, 1H), 7.17 (d, J=8.9 Hz, 1H), 6.70 (s, 1H), 5.28 (q, J=9.2 Hz, 2H), 3.04 (q, J=7.3 Hz, 2H), 2.44 (s, 3H), 1.09 (t, J=7.3 Hz, 3H).

### Example 24

### (N′,N′″E,N′″E)-N′,N′″-(1,3-phenylenebis(methanylylidene))bis(4-((2,3-dihydro-1H-inden-2-yl)amino)benzohydrazide) (Compound 124)

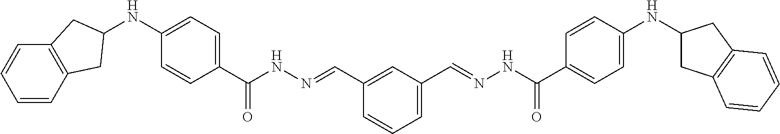 [SMILES: O=C(N/N=C/c1cccc(/C=N/NC(=O)c2ccc(NC3Cc4ccccc4C3)cc2)c1)c1ccc(NC2Cc3ccccc3C2)cc1]

Compound 124 was prepared according to the procedure described in Scheme II from benzene-1,3-dicarboxaldehyde and 4-(2-indanylamino)benzoate. ¹H NMR (500 MHz, DMSO-d₆) δ 11.95 (s, 1H), 7.91 (d, J=8.9 Hz, 1H), 7.17 (d, J=8.9 Hz, 1H), 6.70 (s, 1H), 5.28 (q, J=9.2 Hz, 2H), 3.04 (q, J=7.3 Hz, 2H), 2.44 (s, 3H), 1.09 (t, J=7.3 Hz, 3H).

### Example 25

### (N′,N′″E,N′″E)-N′,N′″-(1,3-phenylenebis(methanylylidene))bis(1-methyl-1H-indole-5-carbohydrazide) (Compound 125)

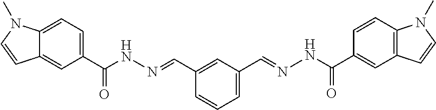 [SMILES: Cn1ccc2cc(C(=O)N/N=C/c3cccc(/C=N/NC(=O)c4ccc5c(ccn5C)c4)c3)ccc21]

Compound 125 was prepared according to the procedure described in Scheme II from benzene-1,3-dicarboxaldehyde and 1-methyl-5-indolecarboxylate.

[M+H]⁺ calcd for C₂₈H₂₄N₆O₂: 477.20. Found: 477.06.

### Example 26

### (N′,N′″E,N′,N′″E)-N′,N′″-((5-(methylamino)-1,3-phenylene)bis(methanylylidene))bis(4-(dimethylamino)benzohydrazide) (Compound 126)

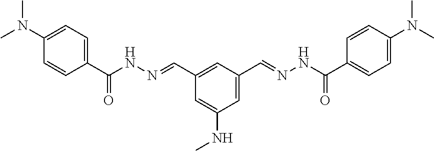

Compound 126 was prepared according to the procedure described in Scheme II from 5-methylaminobenzene-1,3-dicarboxaldehyde and 4-dimethylaminobenzoate. [M+H]⁺ calcd for C₂₇H₃₁N₇O₂: 486.26. Found: 486.03.

### Example 27

### (N′,N′″E,N′″E)-N′,N′″-(1,3-phenylenebis(methanylylidene))bis(4-(pyrrolidin-1-yl)benzohydrazide) (Compound 127)

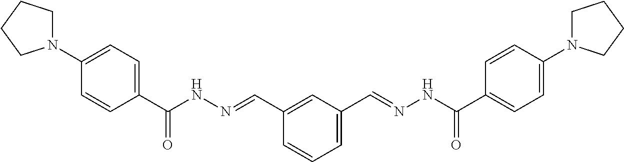 [SMILES: O=C(N/N=C/c1cccc(/C=N/NC(=O)c2ccc(N3CCCC3)cc2)c1)c1ccc(N2CCCC2)cc1]

Compound 127 was prepared according to the procedure described in Scheme II from benzene-1,3-dicarboxaldehyde and 4-pyrrolidinebenzoate. ¹H NMR (500 MHz, DMSO-d₆) δ 11.95 (s, 1H), 7.91 (d, J=8.9 Hz, 1H), 7.17 (d, J=8.9 Hz, 1H), 6.70 (s, 1H), 5.28 (q, J=9.2 Hz, 2H), 3.04 (q, J=7.3 Hz, 2H), 2.44 (s, 3H), 1.09 (t, J=7.3 Hz, 3H).

### Example 28

### (N′,N′″E,N′,N′″E)-N′,N′″-((5-((benzyloxy)methyl)-1,3-phenylene)bis(methanylylidene))bis(4-(dimethylamino)benzohydrazide) (Compound 128)

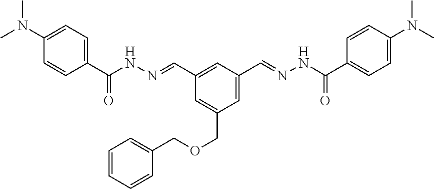

Compound 128 was prepared according to the procedure described in Scheme II from 5-benzyloxymethylbenzene-1,3-dicarboxaldehyde and 4-dimethylaminobenzoate. [M+H]⁺ calcd for C₃₄H₃₆N₆O₃: 577.26. Found: 577.10.

### Example 29

### (N′,N′″E,N′″E)-N′,N′″-(1,3-phenylenebis(methanylylidene))bis(4-(methylamino)benzohydrazide) (Compound 129)

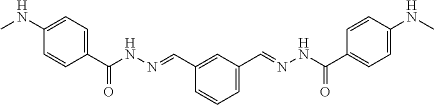

Compound 129 was prepared according to the procedure described in Scheme II from benzene-1,3-dicarboxaldehyde and 4-methylaminobenzoate. ¹H NMR (500 MHz, DMSO-d₆) δ 11.95 (s, 1H), 7.91 (d, J=8.9 Hz, 1H), 7.17 (d, J=8.9 Hz, 1H), 6.70 (s, 1H), 5.28 (q, J=9.2 Hz, 2H), 3.04 (q, J=7.3 Hz, 2H), 2.44 (s, 3H), 1.09 (t, J=7.3 Hz, 3H).

### Example 30

### (N′,N′″E,N′″E)-N′,N′″-(1,3-phenylenebis(methanylylidene))bis(4-((2-hydroxyethyl)amino)benzohydrazide) (Compound 130)

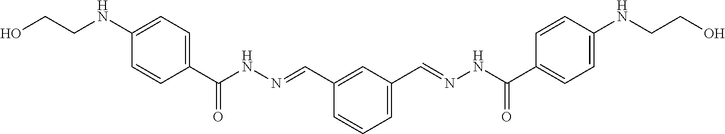 [SMILES: O=C(N/N=C/c1cccc(/C=N/NC(=O)c2ccc(NCCO)cc2)c1)c1ccc(NCCO)cc1]

Compound 130 was prepared according to the procedure described in Scheme II from benzene-1,3-dicarboxaldehyde and 4-(2-hydroxyethylamino)benzoate. ¹H NMR (500 MHz, DMSO-d₆) δ 11.95 (s, 1H), 7.91 (d, J=8.9 Hz, 1H), 7.17 (d, J=8.9 Hz, 1H), 6.70 (s, 1H), 5.28 (q, J=9.2 Hz, 2H), 3.04 (q, J=7.3 Hz, 2H), 2.44 (s, 3H), 1.09 (t, J=7.3 Hz, 3H).

### Example 31

### (N′,N′″E,N′″E)-N′,N′″-(1,3-phenylenebis(methanylylidene))bis(3-(dimethylamino)benzohydrazide) (Compound 131)

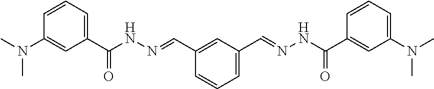

Compound 131 was prepared according to the procedure described in Scheme II from benzene-1,3-dicarboxaldehyde and 3-dimethylaminobenzoate. ¹H NMR (500 MHz, DMSO-d₆) δ 11.82 (s, 2H), 8.52 (s, 2H), 8.11 (s, 1H), 7.76 (d, J=7.5 Hz, 2H), 7.56 (t, J=7.5 Hz, 1H), 7.33 (t, J=8.0 Hz, 2H), 7.19 (d, J=7.0 Hz, 4H), 6.94 (d, J=2.2 Hz, 2H), 2.97 (s, 12H).

### Example 32

### (N′,N′″E,N′″E)-N′,N′″-(1,3-phenylenebis(methanylylidene))bis(4-morpholinobenzohydrazide) (Compound 132)

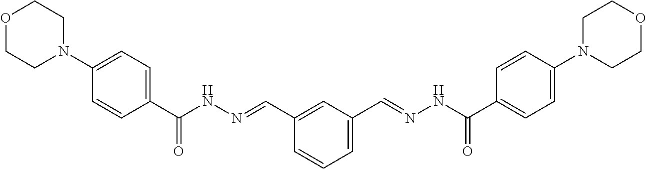 [SMILES: O=C(N/N=C/c1cccc(/C=N/NC(=O)c2ccc(N3CCOCC3)cc2)c1)c1ccc(N2CCOCC2)cc1]

Compound 132 was prepared according to the procedure described in Scheme II from benzene-1,3-dicarboxaldehyde and 4-morpholinobenzoate. [M+H]⁺ calcd for C₃₀H₃₃N₆O₄: 541.26. Found: 541.05

### Example 33

### (N′,N′″E,N′″E)-N′,N′″-(1,3-phenylenebis(methanylylidene))bis(4-(2-oxopyrrolidin-1-yl)benzohydrazide) (Compound 133)

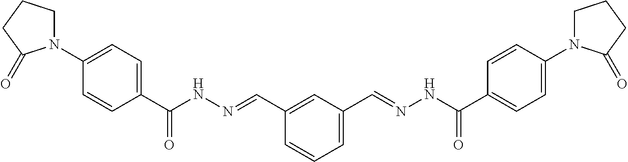 [SMILES: O=C(N/N=C/c1cccc(/C=N/NC(=O)c2ccc(N3CCCC3=O)cc2)c1)c1ccc(N2CCCC2=O)cc1]

Compound 133 was prepared according to the procedure described in Scheme II from benzene-1,3-dicarboxaldehyde and 4-pyrrolidonobenzoate. ¹H NMR (500 MHz, DMSO-d₆) δ 11.93 (s, 2H), 8.47 (s, 2H), 8.17 (s, 1H), 7.95 (d, J=13.7 Hz, 4H), 7.81 (d, J=10 Hz, 4H), 7.77 (d, J=10 Hz, 2H), 7.55 (t, J=10, 12.5 Hz, 1H), 4.28 (m, 4H), 2.53 (m, 4H), 2.07 (m, 4H).

### Example 34

### (N′,N′″E,N′″E)-N′,N′″-(1,3-phenylenebis(methanylylidene))bis(4-(piperazin-1-yl)benzohydrazide) (Compound 134)

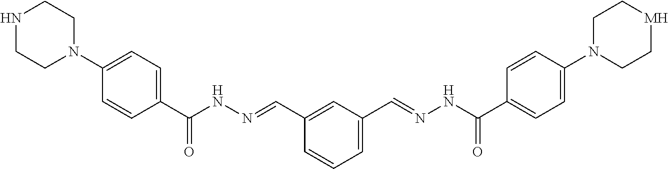

Compound 134 was prepared according to the procedure described in Scheme II from benzene-1,3-dicarboxaldehyde and 4-piperizinylbenzoate. ¹H NMR (500 MHz, DMSO-d₆) δ 11.54 (s, 2H), 10.15 (s, 2H), 8.47 (s, 2H), 8.25 (s, 1H), 7.86 (d, J=12 Hz, 4H), 7.71 (m, 3H), 7.02 (d, J=12 Hz, 4H), 3.17 (m, 8H), 2.86 (m, 8H).

### Example 35

### (N′,N′″E,N′″E)-N′,N′″-((5-((2-hydroxyethyl)amino)-1,3-phenylene)bis(methanylylidene))bis(4-(dimethylamino)benzohydrazide) (Compound 135)

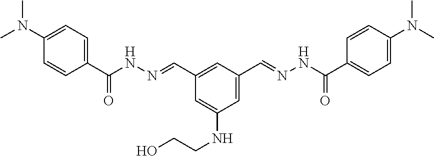

Compound 135 was prepared according to the procedure described in Scheme II from 5-(2-hydroxyethyl)aminobenzene-1,3-dicarboxaldehyde and 4-dimethylaminobenzoate. [M+H]⁺ calcd for C₂₈H₃₃N₇O₃: 516.27. Found: 516.00.

### Example 36

### (N′,N′″E,N′,N′″E)-N′,N′″-(pyridine-3,5-diylbis(methanylylidene))bis(4-morpholinobenzohydrazide) (Compound 136)

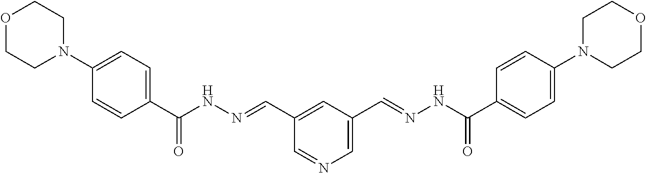 [SMILES: O=C(N/N=C/c1cncc(/C=N/NC(=O)c2ccc(N3CCOCC3)cc2)c1)c1ccc(N2CCOCC2)cc1]

Compound 136 was prepared according to the procedure described in Scheme II from pyridine-3,5-dicarboxaldehyde and 4-morpholinobenzoate. [M+H]⁺ calcd for C₂₉H₃₁N₇O₄: 542.25. Found: 541.99.

### Example 37

### (N′,N′″E,N′,N′″E)-N′,N′″-(pyridine-3,5-diylbis(methanylylidene))bis(4-(dimethylamino)benzohydrazide) (Compound 137)

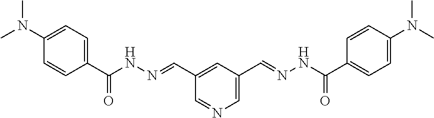

Compound 137 was prepared according to the procedure described in Scheme II from pyridine-3,5-dicarboxaldehyde and 4-dimethylaminobenzoate. ¹H NMR (500 MHz, DMSO-d₆) δ 11.79 (s, 2H), 8.81 (d, J=3.75 Hz, 2H), 8.51 (s, 2H), 8.45 (s, 1H), 7.83 (d, J=15 Hz, 4H), 6.77 (d, J=11.2 Hz, 4H), 3.01 (s, 12H).

### Example 38

### N′,N′″-((5-(benzyloxy)-1,3-phenylene)bis(methylene))bis(4-(dimethylamino)benzohydrazide) (Compound 138)

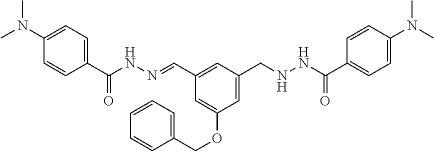

Compound 138 was prepared according to the procedure described in Scheme II from 5-5-benzyloxybenzene-1,3-dicarboxaldehyde and 4-dimethylaminobenzoate. [M+H]⁺ calcd for C₃₃H₂₈N₆O₃: 567.30. Found: 567.17.

### Example 39

### (E)-4-(2-(3-Methoxybenzylidene)hydrazinecarbonyl)-N-(3-((3-methoxyphenethyl)amino)-3-oxopropyl)benzamide (Compound 139)

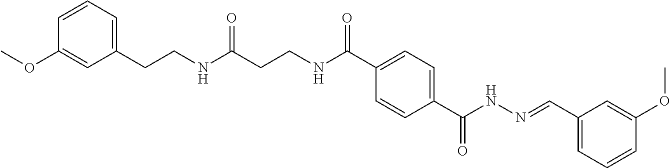

Compound 139 was prepared according to the general procedure described in Scheme I. Preparation of 3-((tert-butoxycarbonyl)amino)propanoic acid: (3-Alanine (Sigma-Aldrich, 1.0 g, 11.2 mmol) and K₂CO₃ (3.1 g, 22.4 mmol) were dissolved in a mixture of dioxane (25 mL) and water (12.5 mL) then the solution was cooled to 0° C. in an ice bath. Di-tert-butyl dicarbonate (2.7 g, 12.3 mmol) was added then the solution was warmed slowly to room temperature and allowed to stir overnight. Upon completion, the solution was acidified with KHSO₄ until pH 3 then extracted with ethyl acetate. The organic layer was washed with brine, dried over Na₂SO₄ then the solvent was removed under reduced pressure to give crude product which was taken forward without further purification.

Preparation of tert-butyl (3-((3-methoxyphenethyl)amino)-3-oxopropyl)carbamate: 3-methoxyphenethylamine (Aldrich, 100 mg, 0.66 mmol), 3-((tert-butoxycarbonyl)amino)-propanoic acid (125 mg, 0.66 mmol), hydroxybenzotriazole (8.9 mg, 0.066 mmol), triethylamine (102 μL, 0.73 mmol) and dimethylormamide (6.6 mL) were combined, then 1-ethyl-3-(3-dimethylaminopropyl)carbodiimide (139 mg, 0.73 mmol) was added. The solution was allowed to stir at room temperature overnight. Upon completion, ethyl acetate and water were added and the layers were separated. The aqueous phase was extracted with ethyl acetate then the combined organic layers were washed with brine and dried over Na₂SO₄. Solvent was removed under reduced pressure to give crude product which was purified by flash chromatography using 0-80% ethyl acetate/hexane as the eluent to give the product as a pure white solid (140 mg, 66%).

Preparation of 3-amino-N-(3-methoxyphenethyl)propanamide: tert-butyl (3-((3-methoxyphenethyl)amino)-3-oxopropyl)carbamate was dissolved in dichloromethane (0.5 mL) then the solution was cooled to 0° C. in an ice bath. Trifluoroacetic acid (300 μL) was added then the solution was allowed to warm slowly to room temperature with stirring. Upon completion of the reaction the solvent is removed under reduced pressure to give crude product which is used without further purification.

Preparation of methyl 4-((3-((3-methoxyphenethyl)amino)-3-oxopropyl)carbamoyl)benzoate: To 3-amino-N-(3-methoxyphenethyl)propanamide (60 mg, 0.27 mmol) was added 4-(methoxycarbonyl)benzoic acid (49 mg, 0.27 mmol), hydroxybenzotriazole (3.7 mg, 0.027 mmol), triethylamine (42 μL, 0.30 mmol) and dimethylormamide (2.7 mL). Lastly, 1-ethyl-3-(3-dimethylaminopropyl)carbodiimide (57 mg, 0.30 mmol) was added and the solution was allowed to stir overnight at room temperature in a capped vial. Upon completion of the reaction an aqueous workup is performed to give the crude product which was purified using flash chromatography.

Preparation of 4-(hydrazinecarbonyl)-N-(3-((3-methoxyphenethyl)amino)-3-oxopropyl)benzamide: Methyl 4-((3-((3-methoxyphenethyl)amino)-3-oxopropyl)carbamoyl)benzoate was treated with methanol (3 mL) and hydrazine hydrate (300 μL), heated to 65° C., then allowed to stir overnight. The reaction is monitored by TLC and upon completion the solvent is removed under reduced pressure to give crude product which is used without purification.

Preparation of Compound 139: To the above crude product (15.0 mg, 0.039 mmol) was added 3-anisaldehyde (9.5 μL, 0.078 mmol), acetic acid (several drops) and ethanol (1 mL). The reaction was allowed to stir overnight at room temperature. The solid product that has formed is isolated by centrifugation then analyzed by LCMS. MS [M+H]⁺ calcd for C₂₈H₃₀N₄O₅: 503.22. Found: 503.04.

### Example 40

### (E)-N-(3-((2-(1H-indol-3-yl)ethyl)amino)-3-oxopropyl)-4-(2-(3-methoxybenzylidene)hydrazinecarbonyl)benzamide (Compound 140)

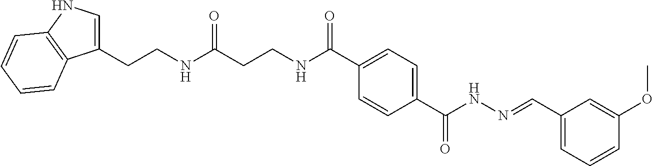 [SMILES: COc1cccc(/C=N/NC(=O)c2ccc(C(=O)NCCC(=O)NCCc3c[nH]c4ccccc34)cc2)c1]

Compound 140 was prepared according to the procedure described in Scheme I from 4-(2-(3-indolylethyl)aminocarbonyl)ethylaminocarbonyl)benzoate and 3-methoxybenzaldehyde. [M+H]⁺ calcd for C₂₉H₂₉N₅O₄: 512.22. Found: 512.03.

### Example 41

### (E)-N-(3-((2-hydroxyethyl)amino)-3-oxopropyl)-4-(2-(3-methoxybenzylidene)hydrazinecarbonyl)benzamide (Compound 141)

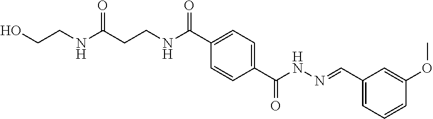 [SMILES: COc1cccc(/C=N/NC(=O)c2ccc(C(=O)NCCC(=O)NCCO)cc2)c1]

Compound 141 was prepared according to the procedure described in Scheme I from 4-(2-(2-hydroxyethylaminocarbonyl)ethylaminocarbonyl)benzoate and 3-methoxybenzaldehyde. [M+H]⁺ calcd for C₂₁H₂₄N₄O₅: 413.17. Found: 413.00.

### Example 42

### 4-((E)-2-(3-((E)-2-chlorostyryl)benzylidene)hydrazinecarbonyl)-N-(3-((2-hydroxyethyl)amino)-3-oxopropyl)benzamide (Compound 142)

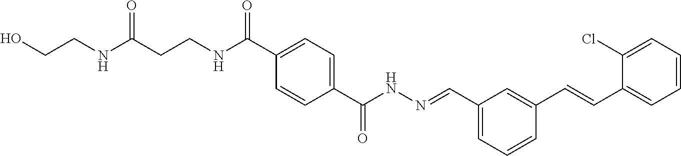 [SMILES: O=C(CCNC(=O)c1ccc(C(=O)N/N=C/c2cccc(/C=C/c3ccccc3Cl)c2)cc1)NCCO]

Compound 142 was prepared according to the procedure described in Scheme I from 4-(2-(2-hydroxyethylaminocarbonyl)ethylaminocarbonyl)benzoate and 3-(2E-(2-chlorophenyl)ethenyl)benzaldehyde. [M+H]⁺ calcd for C₂₈H₂₇ClN₄O₄: 519.17. Found: 519.00.

### Example 43

### (E)-4-(2-(4-fluoro-3-methoxybenzylidene)hydrazinecarbonyl)-N-(3-((2-hydroxyethyl)amino)-3-oxopropyl)benzamide (Compound 143)

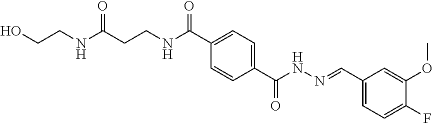 [SMILES: COc1cc(/C=N/NC(=O)c2ccc(C(=O)NCCC(=O)NCCO)cc2)ccc1F]

Compound 143 was prepared according to the procedure described in Scheme I from 4-(2-(2-hydroxyethylaminocarbonyl)ethylaminocarbonyl)benzoate and 4-fluoro-3-methoxybenzaldehyde. [M+H]⁺ calcd for C₂₁H₂₃FN₄O₅: 431.17. Found: 431.00.

### Example 44

### (E)-4-(2-(2-hydroxy-5-methoxybenzylidene)hydrazinecarbonyl)-N-(3-((2-hydroxyethyl)amino)-3-oxopropyl)benzamide (Compound 144)

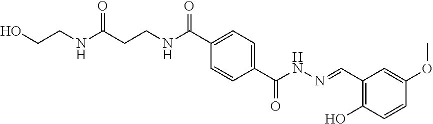 [SMILES: COc1ccc(O)c(/C=N/NC(=O)c2ccc(C(=O)NCCC(=O)NCCO)cc2)c1]

Compound 144 was prepared according to the procedure described in Scheme I from 4-(2-(2-hydroxyethylaminocarbonyl)ethylaminocarbonyl)benzoate and 2-hydroxy-5-methoxybenzaldehyde. [M+H]⁺ calcd for C₂₁H₂₄N₄O₆: 429.17. Found: 429.00

### Example 45

### (E)-4-(2-(3,5-dimethoxybenzylidene)hydrazinecarbonyl)-N-(3-((2-hydroxyethyl)amino)-3-oxopropyl)benzamide (Compound 145)

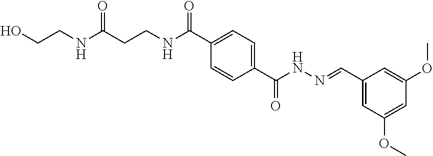 [SMILES: COc1cc(/C=N/NC(=O)c2ccc(C(=O)NCCC(=O)NCCO)cc2)cc(OC)c1]

Compound 145 was prepared according to the procedure described in Scheme I from 4-(2-(2-hydroxyethylaminocarbonyl)ethylaminocarbonyl)benzoate and 3,5-dimethoxybenzaldehyde. [M+H]⁺ calcd for C₂₂H₂₆N₄O₆: 443.19. Found: 442.91.

### Example 46

### (E)-4-(2-(4-hydroxy-3-methoxybenzylidene)hydrazinecarbonyl)-N-(3-((2-hydroxyethyl)amino)-3-oxopropyl)benzamide (Compound 146)

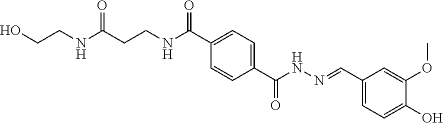 [SMILES: COc1cc(/C=N/NC(=O)c2ccc(C(=O)NCCC(=O)NCCO)cc2)ccc1O]

Compound 146 was prepared according to the procedure described in Scheme I from 4-(2-(2-hydroxyethylaminocarbonyl)ethylaminocarbonyl)benzoate and 4-hydroxy-5-methoxybenzaldehyde. [M+H]⁺ calcd for C₂₁H₂₄N₄O₆: 429.17. Found: 428.88.

### Example 47

### (E)-N-(3-((2-hydroxyethyl)amino)-3-oxopropyl)-4-(2-(3-methoxy-4-methylbenzylidene)hydrazinecarbonyl)benzamide (Compound 147)

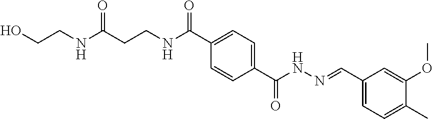 [SMILES: COc1cc(/C=N/NC(=O)c2ccc(C(=O)NCCC(=O)NCCO)cc2)ccc1C]

Compound 147 was prepared according to the procedure described in Scheme I from 4-(2-(2-hydroxyethylaminocarbonyl)ethylaminocarbonyl)benzoate and 3-methoxy-4-methylbenzaldehyde. [M+H]⁺ calcd for C₂₂H₂₆N₄O₅: 427.19. Found: 426.92.

### Example 48

### (E)-N-(2-hydroxyethyl)-4-((4-(2-(3-methoxybenzylidene)hydrazinecarbonyl)phenyl)amino)butanamide (Compound 148)

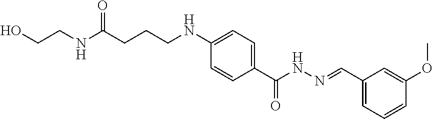 [SMILES: COc1cccc(/C=N/NC(=O)c2ccc(NCCCC(=O)NCCO)cc2)c1]

Compound 148 was prepared according to the procedure described in Scheme I from 4-(3-(2-hydroxyethylaminocarbonyl)propylamino)benzoate and 3-methoxybenzaldehyde. [M+H]⁺ calcd for C₂₁H₂₆N₄O₄: 399.20. Found: 399.47.

### Example 49

### (E)-4-(2-(3-(dimethylamino)benzylidene)hydrazinecarbonyl)-N-(3-((2-hydroxyethyl)amino)-3-oxopropyl)benzamide (Compound 149)

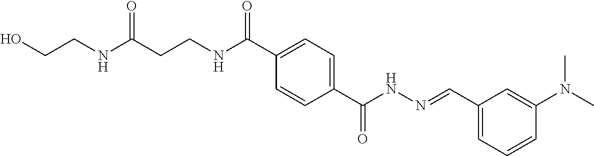 [SMILES: CN(C)c1cccc(/C=N/NC(=O)c2ccc(C(=O)NCCC(=O)NCCO)cc2)c1]

Compound 149 was prepared according to the procedure described in Scheme I from 4-(2-(2-hydroxyethylaminocarbonyl)ethylaminocarbonyl)benzoate and 3-dimethylaminobenzaldehyde. [M+H]⁺ calcd for C₂₂H₂₇N₅O₄: 426.21. Found: 426.53.

### Example 50

### (E)-4-((4-(2-(2-hydroxy-5-methoxybenzylidene)hydrazinecarbonyl)phenyl)amino)-N-(2-hydroxyethyl)butanamide (Compound 150)

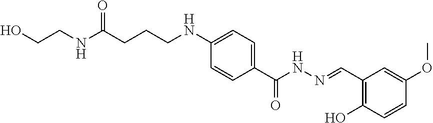 [SMILES: COc1ccc(O)c(/C=N/NC(=O)c2ccc(NCCCC(=O)NCCO)cc2)c1]

Compound 150 was prepared according to the procedure described in Scheme I from 4-(3-(2-hydroxyethylaminocarbonyl)propylamino)benzoate and 2-hydroxy-5-methoxybenzaldehyde. [M+H]⁺ calcd for C₂₁H₂₆N₄O₅: 415.19. Found: 415.53.

### Example 51

### (E)-4-((4-(2-(3-(dimethylamino)benzylidene)hydrazinecarbonyl)phenyl)amino)-N-(2-hydroxyethyl)butanamide (Compound 151)

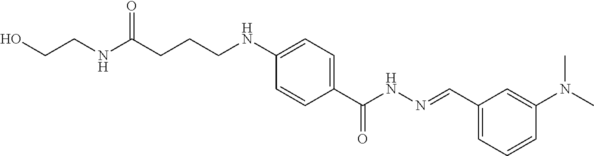 [SMILES: CN(C)c1cccc(/C=N/NC(=O)c2ccc(NCCCC(=O)NCCO)cc2)c1]

Compound 151 was prepared according to the procedure described in Scheme I from 4-(3-(2-hydroxyethylaminocarbonyl)propylamino)benzoate and 3-dimethylaminobenzaldehyde. [M+H]⁺ calcd for C₂₂H₂₉N₅O₃: 412.23. Found: 412.56.

### Example 52

### (E)-4-((4-(2-(3-(2-hydroxyethoxy)benzylidene)hydrazinecarbonyl)phenyl)amino)-N-(2-hydroxyethyl)butanamide (Compound 152)

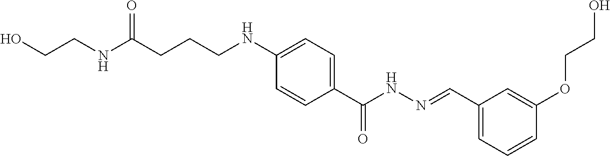 [SMILES: O=C(CCCNc1ccc(C(=O)N/N=C/c2cccc(OCCO)c2)cc1)NCCO]

Compound 152 was prepared according to the procedure described in Scheme I from 4-(3-(2-hydroxyethylaminocarbonyl)propylamino)benzoate and 3-(2-hydroxyethoxy)benzaldehyde. [M+H]⁺ calcd for C₂₂H₂₈N₄O₅: 429.21. Found: 429.50.

### Example 53

### (E)-4-((4-(2-(3,5-dimethoxybenzylidene)hydrazinecarbonyl)phenyl)amino)-N-(2-hydroxyethyl)butanamide (Compound 153)

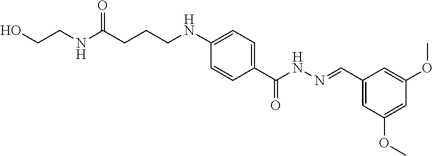 [SMILES: COc1cc(/C=N/NC(=O)c2ccc(NCCCC(=O)NCCO)cc2)cc(OC)c1]

Compound 153 was prepared according to the procedure described in Scheme I from 4-(3-(2-hydroxyethylaminocarbonyl)propylamino)benzoate and 3,5-dimethoxybenzaldehyde. [M+H]⁺ calcd for C₂₂H₂₈N₄O₅: 429.21. Found: 429.50.

### Example 54

### (E)-4-((4-(2-((1H-indol-6-yl)methylene)hydrazinecarbonyl)phenyl)amino)-N-(2-hydroxyethyl)butanamide (Compound 154)

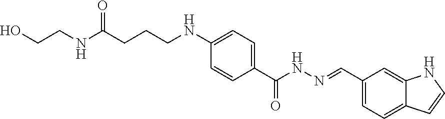 [SMILES: O=C(CCCNc1ccc(C(=O)N/N=C/c2ccc3cc[nH]c3c2)cc1)NCCO]

Compound 154 was prepared according to the procedure described in Scheme I from 4-(3-(2-hydroxyethylaminocarbonyl)propylamino)benzoate and 6-indolecarboxaldehyde. [M+H]⁺ calcd for C₂₂H₂₅N₅O₃: 408.20. Found: 408.51.

### Example 55

### (E)-4-(5-(2-(3-methoxybenzylidene)hydrazinecarbonyl)-1H-indol-1-yl)-N-(2-methoxyethyl)butanamide (Compound 155)

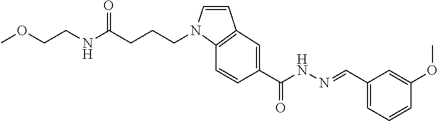

Compound 155 was prepared according to the procedure described in Scheme I from 1-(3-(2-methoxyethyl)aminocarbonylpropyl)indole-5-carboxylate and 3-methoxybenzaldehyde. ¹H NMR (500 MHz, DMSO-d₆) δ 11.77 (s, 1H), 8.43 (s, 1H), 8.20 (d, J=1.5 Hz, 1H), 7.89 (t, J=5.5 Hz, 1H), 7.72 (dd, J=2, 9 Hz, 1H), 7.57 (d, J=9 Hz, 1H), 7.46 (d, J=3 Hz, 1H), 7.36 (t, J=8 Hz, 1H), 7.27 (s, 2H), 6.99 (d, J=8 Hz, 1H), 6.58 (d, J=2.5 Hz, 1H), 4.20 (t, J=6.5 Hz, 2H), 3.80 (s, 3H), 3.32-3.29 (m, 2H), 3.20-3.14 (m, 5H), 2.05 (t, J=8 Hz, 2H), 1.96 (p, J=7 Hz, 2H).

### Example 56

### (E)-N-(3-(dimethylamino)phenyl)-4-(5-(2-(3-methoxybenzylidene)hydrazinecarbonyl)-1H-indol-1-yl)butanamide (Compound 156)

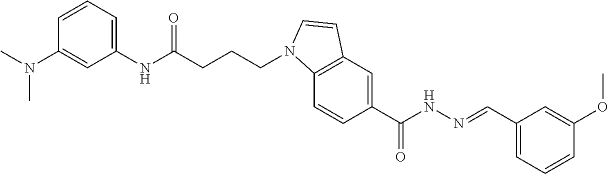

Compound 156 was prepared according to the procedure described in Scheme I from 1-(3-(3-dimethylaminophenyl)aminocarbonylpropyl)indole-5-carboxylate and 3-methoxybenzaldehyde. [M+H]⁺ calcd for C₂₉H₃₁N₅O₃: 498.24. Found: 497.98.

### Example 57

### (E)-N-(4-(dimethylamino)phenyl)-4-(5-(2-(3-methoxybenzylidene)hydrazinecarbonyl)-1H-indol-1-yl)butanamide (Compound 157)

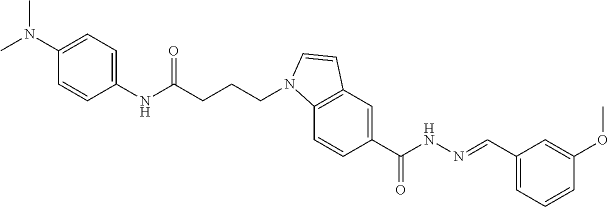

Compound 157 was prepared according to the procedure described in Scheme I from 1-(3-(4-dimethylaminophenyl)aminocarbonylpropyl)indole-5-carboxylate and 3-methoxybenzaldehyde. ¹H NMR (500 MHz, DMSO-d₆) δ 11.77 (s, 1H), 9.58 (s, 1H), 8.43 (bs, 1H), 8.21 (d, J=1.0 Hz, 1H), 7.73 (dd, J=1.5, 8.5 Hz, 1H), 7.60 (d, J=8.5 Hz, 1H), 7.50 (d, J=2.5 Hz, 1H), 7.44 (m, 1H), 7.36 (d, J=9 Hz, 2H), 7.26 (m, 2H), 6.99 (d, J=8 Hz, 1H), 6.75 (d, J=9 Hz, 1H), 6.65 (d, J=9 Hz, 2H), 6.60 (d, J=2.5 Hz, 1H), 4.27 (t, J=7 Hz, 2H), 3.80 (s, 3H), 2.81 (s, 6H), 2.23 (t, J=7.5 Hz, 2H), 2.06 (p, J=7.5 Hz, 2H).

### Example 58

### (E)-4-(5-(2-(3-(1H-pyrazol-1-yl)benzylidene)hydrazinecarbonyl)-1H-indol-1-yl)-N-(3-(dimethylamino)phenyl)butanamide (Compound 158)

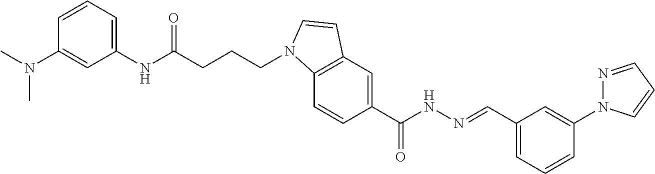 [SMILES: CN(C)c1cccc(NC(=O)CCCn2ccc3cc(C(=O)N/N=C/c4cccc(-n5cccn5)c4)ccc32)c1]

Compound 158 was prepared according to the procedure described in Scheme I from 1-(3-(3-dimethylaminophenyl)aminocarbonylpropyl)indole-5-carboxylate and 3-(pyrazol-1-yl)benzaldehyde. [M+H]⁺ calcd for C₃₁H₃₁N₇O₂: 534.25. Found: 534.00.

### Example 59

### (E)-4-(5-(2-(3-(1H-pyrazol-1-yl)benzylidene)hydrazinecarbonyl)-1H-indol-1-yl)-N-(4-(dimethylamino)phenyl)butanamide (Compound 159)

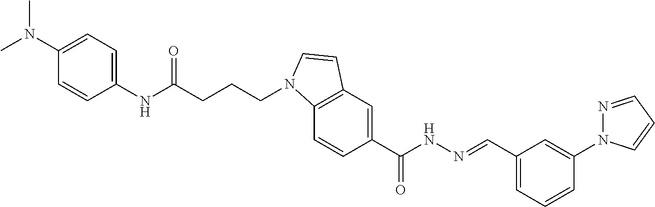 [SMILES: CN(C)c1ccc(NC(=O)CCCn2ccc3cc(C(=O)N/N=C/c4cccc(-n5cccn5)c4)ccc32)cc1]

Compound 159 was prepared according to the procedure described in Scheme I from 1-(3-(4-dimethylaminophenyl)aminocarbonylpropyl)indole-5-carboxylate and 3-(pyrazol-1-yl)benzaldehyde. [M+H]⁺ calcd for C₃₁H₃₁N₇O₂: 534.25. Found: 533.97.

### Example 60

### (E)-4-(5-(2-(3-(1H-pyrazol-1-yl)benzylidene)hydrazinecarbonyl)-1H-indol-1-yl)-N-(2-methoxyethyl)butanamide (Compound 160)

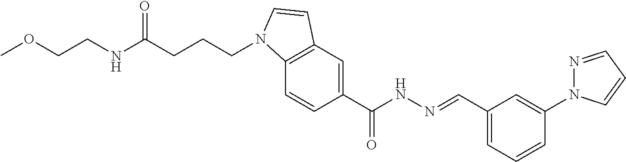 [SMILES: COCCNC(=O)CCCn1ccc2cc(C(=O)N/N=C/c3cccc(-n4cccn4)c3)ccc21]

Compound 160 was prepared according to the procedure described in Scheme I from 1-(3-(2-methoxyethyl)aminocarbonylpropyl)indole-5-carboxylate and 3-(pyrazol-1-yl)benzaldehyde. [M+H]⁺ calcd for C₂₆H₂₈N₆O₃: 473.22. Found: 472.94.

### Example 61

### (E)-4-(5-(2-(3-(1H-pyrazol-1-yl)benzylidene)hydrazinecarbonyl)-1H-indol-1-yl)-N-(2-acetamidoethyl)butanamide (Compound 161)

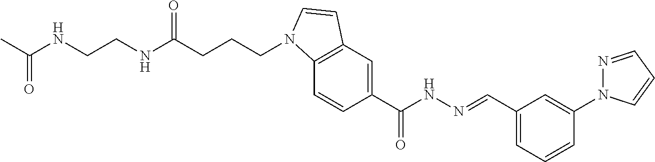 [SMILES: CC(=O)NCCNC(=O)CCCn1ccc2cc(C(=O)N/N=C/c3cccc(-n4cccn4)c3)ccc21]

Compound 161 was prepared according to the procedure described in Scheme I from 1-(3-(2-acetylamidoethyl)aminocarbonylpropyl)indole-5-carboxylate and 3-(pyrazol-1-yl)benzaldehyde. [M+H]⁺ calcd for C₂₇H₂₉N₇O₃: 500.23. Found: 499.94.

### Example 62

### (E)-N-(2-acetamidoethyl)-4-(5-(2-(3-methoxybenzylidene)hydrazinecarbonyl)-1H-indol-1-yl)butanamide (Compound 162)

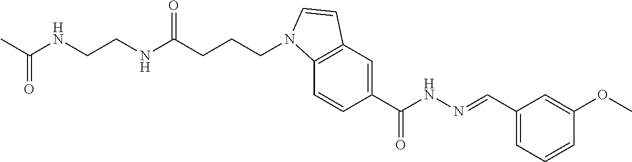 [SMILES: COc1cccc(/C=N/NC(=O)c2ccc3c(ccn3CCCC(=O)NCCNC(C)=O)c2)c1]

Compound 162 was prepared according to the procedure described in Scheme I from 1-(3-(2-acetylamidoethyl)aminocarbonylpropyl)indole-5-carboxylate and 3-methoxybenzaldehyde. [M+H]⁺ calcd for C₂₅H₂₉N₅O₄: 464.22. Found: 463.93.

### Example 63

### (E)-4-(5-(2-(3-methoxybenzylidene)hydrazinecarbonyl)-1H-indol-1-yl)-N-(2,2,2-trifluoroethyl)butanamide (Compound 163)

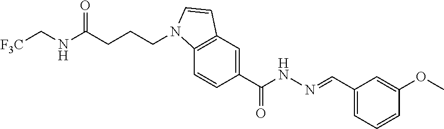 [SMILES: COc1cccc(/C=N/NC(=O)c2ccc3c(ccn3CCCC(=O)NCC(F)(F)F)c2)c1]

Compound 163 was prepared according to the procedure described in Scheme I from 1-(3-(2,2,2-trifluoroethyl)aminocarbonylpropyl)indole-5-carboxylate and 3-methoxybenzaldehyde. [M+H]⁺ calcd for C₂₃H₂₃F₃N₄O₃: 461.17. Found: 460.92.

### Example 64

### (E)-N-(2-hydroxyethyl)-4-(5-(2-(3-methoxybenzylidene)hydrazinecarbonyl)-1H-indol-1-yl)butanamide (Compound 164)

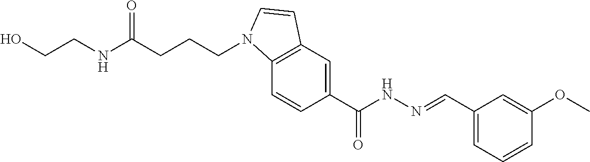 [SMILES: COc1cccc(/C=N/NC(=O)c2ccc3c(ccn3CCCC(=O)NCCO)c2)c1]

Compound 164 was prepared according to the procedure described in Scheme I from 1-(3-(2-hydroxyethyl)aminocarbonylpropyl)indole-5-carboxylate and 3-methoxybenzaldehyde. [M+H]⁺ calcd for C₂₃H₂₆N₄O₄: 423.20. Found: 422.88.

### Example 65

### (E)-N-(3-((4-hydroxybutyl)amino)-3-oxopropyl)-4-(2-(3-methoxybenzylidene)hydrazinecarbonyl)benzamide (Compound 165)

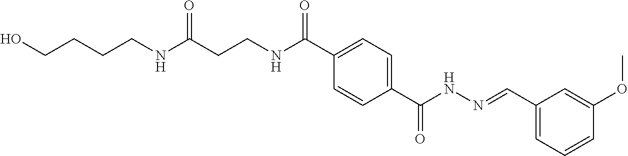 [SMILES: COc1cccc(/C=N/NC(=O)c2ccc(C(=O)NCCC(=O)NCCCCO)cc2)c1]

Compound 165 was prepared according to the procedure described in Scheme I from 4-(2-(4-hydroxybutylaminocarbonyl)ethylaminocarbonyl)benzoate and 3-methoxybenzaldehyde. [M+H]⁺ calcd for C₂₃H₂₈N₄O₅: 441.21. Found: 441.01.

### Example 66

### (E)-N-(2-(dimethylamino)ethyl)-4-(5-(2-(3-methoxybenzylidene)hydrazinecarbonyl)-1H-indol-1-yl)butanamide (Compound 166)

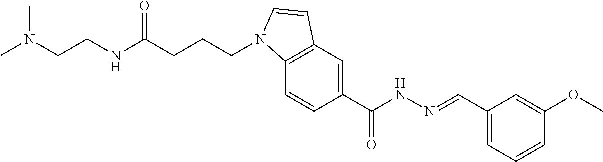

Compound 166 was prepared according to the procedure described in Scheme I from 1-(3-(2-dimethylaminoethyl)aminocarbonylpropyl)indole-5-carboxylate and 3-methoxybenzaldehyde. ¹H NMR (500 MHz, Acetone-d₆) δ 11.09 (bs, 1H), 8.51 (bs, 1H), 8.26 (d, J=19 Hz, 2H), 7.83 (dd, J=1.5, 8.5 Hz, 1H), 7.39 (bs, 1H), 7.35 (t, J=7.5 Hz, 1H), 7.30 (m, 1H), 7.08 (s, 1H), 6.99-6.97 (m, 1H), 6.58 (d, J=3 Hz, 1H), 4.31 (t, J=7 Hz, 2H), 3.85 (s, 3H), 3.39 (q, J=6 Hz, 2H), 2.67 (t, J=6 Hz, 2H), 2.41 (s, 6H), 2.18-2.11 (m, 4H).

### Example 67

### Methyl 4-(2-(4-morpholinophenyl)-1H-benzo[d]imidazole-5-carboxamido)benzoate (Compound 167)

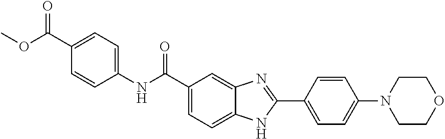 [SMILES: COC(=O)c1ccc(NC(=O)c2ccc3[nH]c(-c4ccc(N5CCOCC5)cc4)nc3c2)cc1]

Compound 167 was prepared according to the general procedure similar to that described in Scheme III. Preparation of 2-(4-morpholinophenyl)-1H-benzo[d]imidazole-5-carboxylic acid: 4-Morpholinobenzaldehyde (475 mg, 2.5 mmole), sodium metabisulfite (85 mg) and 3,4-diaminobenzoic acid (152 mg, 2.7 mmole) were placed in a 10-mL microwave tube and 5.0 mL HPLC grade 2-propanol added. The reaction mixture was microwaved at 170° C. for 55 minutes. The reaction mixture was added dropwise to 25 mL water and stirred at room temperature for 30 min. then it was filtered and washed with plenty of water, ethyl acetate and hexanes and dried to provide the pure intermediate acid (323 mg, 40% yield). This compound was used for next step without any further purification.

Preparation of Compound 167: 2-(4-Morpholinophenyl)-1H-benzo[d]imidazole-5-carboxylic acid (32 mg, 0.1 mmol) and N-(3-dimethylaminopropyl)-N′-ethylcarbodiimide hydrochloride (EDC) (40 mg, 0.2 mmole) were place in a 20 mL vial and pyridine (1.0 mL) added and capped tightly. The reaction mixture was stirred at room temperature for overnight. The reaction mixture was evaporated to dryness and the residue was washed thoroughly with plenty of water, hexanes and EtOAc then dried. Crystallization out of methanol/water provided compound 167 in 25% yield (11.4 mg). [M+H]⁺ calcd for C₂₆H₂₄N₄O₄: 457.18. Found: 456.94.

### Example 68

### 2-(4-Morpholinophenyl)-N-(3-((4-morpholinophenyl)amino)-3-oxopropyl)-1H-benzo[d]imidazole-5-carboxamide (Compound 168)

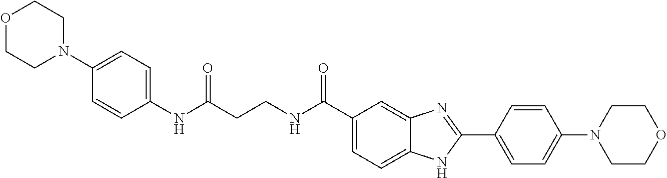 [SMILES: O=C(CCNC(=O)c1ccc2[nH]c(-c3ccc(N4CCOCC4)cc3)nc2c1)Nc1ccc(N2CCOCC2)cc1]

Compound 168 was prepared according to the procedure similar to that described in Scheme III from the 3,4-dinitrobenzamide and 4-morpholinobenzaldehyde.

[M+H]⁺ calcd for C₃₁H₃₄N₆O₄: 555.26. Found: 555.03.

### Example 69

### N-(2-(4-Methyl-3,4-dihydro-2H-benzo[b][1,4]oxazin-7-yl)-1H-benzimidazol-5-yl)-4-morpholinobenzamide (Compound 169)

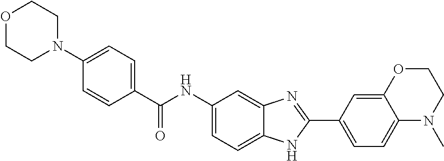 [SMILES: CN1CCOc2cc(-c3nc4cc(NC(=O)c5ccc(N6CCOCC6)cc5)ccc4[nH]3)ccc21]

Compound 169 was prepared according to the procedure similar to that described in Scheme III from 5-amino-2-(4-methyl-3,4-dihydrobenzoxazin-7-yl)benzimidazole and 4-morpholinobenzoic acid. [M+H]⁺ calcd for C₂₇H₂₇N₅O₃: 470.21. Found: 496.96.

### Example 70

### N-(2-(4-Phenoxyphenyl)-1H-benzo[d]imidazol-5-yl)benzamide (Compound 170)

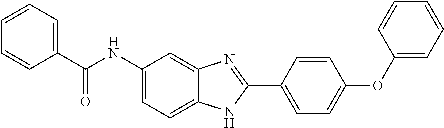 [SMILES: O=C(Nc1ccc2[nH]c(-c3ccc(Oc4ccccc4)cc3)nc2c1)c1ccccc1]

Compound 170 was prepared according to the procedure similar to that described in Scheme III from 3,4-dinitro-N-benzoaniline and 4-phenoxybenzaldehyde. [M+H]⁺ calcd for C₂₆H₁₉N₃O₂: 406.16. Found: 406.08.

### Example 71

### N-(2-(6-Methoxynaphthalen-2-yl)-1H-benzo[d]imidazol-5-yl)benzamide (Compound 171)

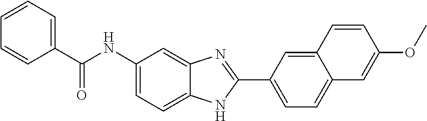 [SMILES: COc1ccc2cc(-c3nc4cc(NC(=O)c5ccccc5)ccc4[nH]3)ccc2c1]

Compound 171 was prepared according to the procedure similar to that described in Scheme III from 3,4-dinitro-N-benzoaniline and 6-methoxynaphthalene-2-carboxaldehyde. [M+H]⁺ calcd for C₂₅H₁₉N₃O₂: 394.15. Found: 394.10.

### Example 72

### N-(2-Phenyl-1H-benzo[d]imidazol-5-yl)-[1,1′-biphenyl]-4-carboxamide (Compound 172)

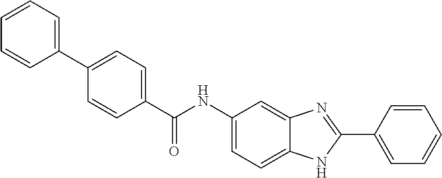 [SMILES: O=C(Nc1ccc2[nH]c(-c3ccccc3)nc2c1)c1ccc(-c2ccccc2)cc1]

Compound 172 was prepared according to the procedure similar to that described in Scheme III from 3,4-dinitro-N-(4-phenylbenzo)aniline and benzaldehyde. ¹H NMR (500 MHz, DMSO-d₆) δ 10.62 (s, 1H), 8.83 (m, 1H), 8.52 (d, J=1.5 Hz, 1H), 8.23 (m, 2H), 8.13 (d, J=8.5 Hz, 2H), 7.82 (d, J=9 Hz, 3H), 7.79 (dd, J=1.5, 9 Hz, 1H), 7.70 (dd, J=1.5, 9 Hz, 2H), 7.73 (m, 3H), 7.53 (t, J=7.5 Hz, 2H), 7.44 (dt, J=1, 7.5 Hz, 1H).

### Example 73

### N-(2-(4-(pyrimidin-5-yl)phenyl)-1H-benzo[d]imidazol-5-yl)benzamide (Compound 173)

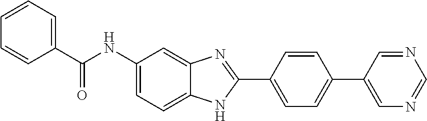 [SMILES: O=C(Nc1ccc2[nH]c(-c3ccc(-c4cncnc4)cc3)nc2c1)c1ccccc1]

Compound 173 was prepared according to the procedure similar to that described in Scheme III from 3,4-dinitro-N-benzoaniline and 4-(pyrimidin-5-yl)benzaldehyde. [M+H]⁺ calcd for C₂₄H₁₇N₅O: 392.15. Found: 391.91.

### Example 74

### N-(2-(4-(pyridin-3-yl)phenyl)-1H-benzo[d]imidazol-5-yl)benzamide (Compound 174)

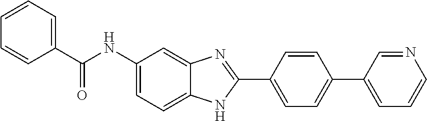 [SMILES: O=C(Nc1ccc2[nH]c(-c3ccc(-c4cccnc4)cc3)nc2c1)c1ccccc1]

Compound 174 was prepared according to the procedure similar to that described in Scheme III from 3,4-dinitro-N-benzoaniline and 4-(pyridyl-3-)benzaldehyde. [M+H]⁺ calcd for C₂₅H₁₈N₄O: 391.16. Found: 390.90.

### Example 75

### 2′-(4-Ethoxyphenyl)-5-(4-methylpiperazin-1-yl)-1H,1H-2,5′-bibenzo[d]imidazole (Compound 175)

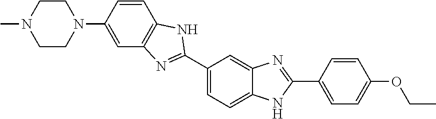 [SMILES: CCOc1ccc(-c2nc3cc(-c4nc5cc(N6CCN(C)CC6)ccc5[nH]4)ccc3[nH]2)cc1]

Compound 175 was prepared according to the procedure similar to that described in Scheme III from 5-(N-methylpiperizinyl)-2-(3,4-diaminophenyl)benzimidazole and 4-ethoxybenzaldehyde.

### Example 76

### 3-Bromo-N-(2-phenyl-1H-benzo[d]imidazol-5-yl)benzamide (Compound 176)

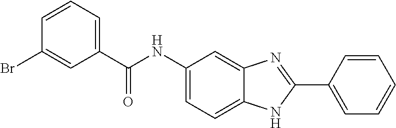 [SMILES: O=C(Nc1ccc2[nH]c(-c3ccccc3)nc2c1)c1cccc(Br)c1]

Compound 176 was prepared according to the procedure similar to that described in Scheme III from 3,4-dinitro-N-(3-bromobenzo)aniline and benzaldehyde. ¹H NMR (300 MHz, Acetone-d₆) δ 9.77 (bs, 1H), 8.37 (s, 1H), 8.23 (d, J=7 Hz, 2H), 8.18 (s, 1H), 8.04 (m, 1H), 7.75 (d, J=8.5 Hz, 1H), 7.62-7.46 (m, 7H).

### Example 77

### 4-Methoxy-N-(2-(4-(pyrrolidin-1-yl)phenyl)-1H-benzo[d]imidazol-5-yl)benzamide (Compound 177)

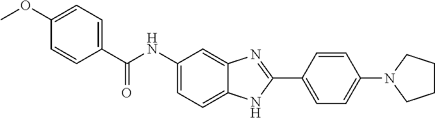 [SMILES: COc1ccc(C(=O)Nc2ccc3[nH]c(-c4ccc(N5CCCC5)cc4)nc3c2)cc1]

Compound 177 was prepared according to the procedure similar to that described in Scheme III from 3,4-dinitro-N-(4-methoxybenzo)aniline and 4-(pyrrolidin-1-yl)benzaldehyde. [M+H]⁺ calcd for C₂₅H₂₄N₄O₂: 413.20. Found: 413.51.

### Example 78

### 4-Guanidino-N-(2-(4-(pyrrolidin-1-yl)phenyl)-1H-benzo[d]imidazol-5-yl)benzamide (Compound 178)

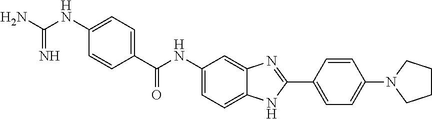 [SMILES: N=C(N)Nc1ccc(C(=O)Nc2ccc3[nH]c(-c4ccc(N5CCCC5)cc4)nc3c2)cc1]

Compound 178 was prepared according to the procedure similar to that described in Scheme III from 3,4-dinitro-N-(4-amidinoaminobenzo)aniline and 4-(pyrrolidin-1-yl)benzaldehyde. [M+H]⁺ calcd for C₂₅H₂₅N₇O: 440.22. Found: 440.57.

### Example 79

### 4-(Dimethylamino)-N-(2-(4-(2-oxopyrrolidin-1-yl)phenyl)-1H-benzo[d]imidazol-5-yl)benzamide (Compound 179)

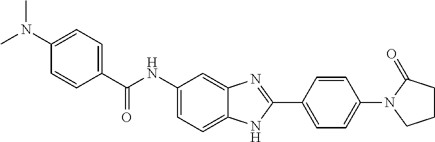 [SMILES: CN(C)c1ccc(C(=O)Nc2ccc3[nH]c(-c4ccc(N5CCCC5=O)cc4)nc3c2)cc1]

Compound 179 was prepared according to the procedure similar to that described in Scheme III from 3,4-dinitro-N-(4-dimethylaminobenzo)aniline and 4-(pyrrolidon-1-yl)benzaldehyde. [M+H]⁺ calcd for C₂₆H₂₅N₅O₂: 440.20. Found: 440.50.

### Example 80

### 4-(Dimethylamino)-N-(2-(phenylamino)-1H-benzo[d]imidazol-5-yl)benzamide (Compound 180)

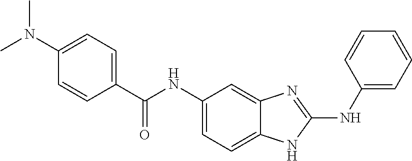 [SMILES: CN(C)c1ccc(C(=O)Nc2ccc3[nH]c(Nc4ccccc4)nc3c2)cc1]

Compound 180 was prepared according to the procedure similar to that described in Scheme III from 4-dimethylaminobenzoic acid and 5-amino-2-phenylaminobenzimidazole. [M+H]⁺ calcd for C₂₂H₂₂N₅O: 372.18. Found: 371.95.

### Example 81

### 4-(Dimethylamino)-N-(2-(4-methyl-3,4-dihydro-2H-benzo[b][1,4]oxazin-7-yl)-1H-benzo[d]imidazol-5-yl)benzamide (Compound 181)

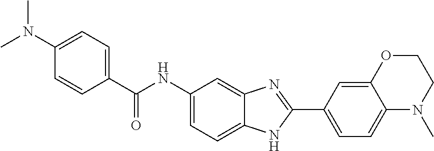

Compound 181 was prepared according to the procedure similar to that described in Scheme III from 3,4-dinitro-N-(4-dimethylaminobenzoyl)aniline and 1-methyl-2,3-dihydrobenzo[1,4]oxazine-6-carboxaldehyde. [M+H]⁺ calcd for C₂₅H₂₅N₅O₂: 428.20. Found: 428.49.

### Example 82

### N-(4-(5-(cyclopent-1-enecarboxamido)-1H-benzo[d]imidazol-2-yl)phenyl)cyclopent-1-enecarboxamide (Compound 182)

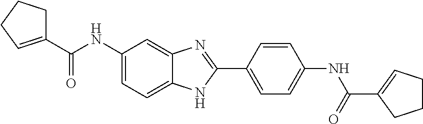 [SMILES: O=C(Nc1ccc(-c2nc3cc(NC(=O)C4=CCCC4)ccc3[nH]2)cc1)C1=CCCC1]

Compound 182 was prepared according to the procedure similar to that described in Scheme III from 5-amino-2-(4-aminophenyl)benzimidazole and 1-cyclohexenecarboxylic acid. [M+H]⁺ calcd for C₂₅H₂₄N₄O₂: 413.20. Found: 412.96.

### Example 83

### N-(2-(2,3-dihydrobenzofuran-5-yl)-1H-benzo[d]imidazol-5-yl)-4-(dimethylamino)benzamide (Compound 183)

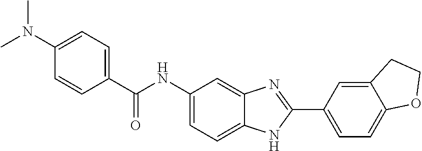 [SMILES: CN(C)c1ccc(C(=O)Nc2ccc3[nH]c(-c4ccc5c(c4)CCO5)nc3c2)cc1]

Compound 183 was prepared according to the procedure similar to that described in Scheme III from 3,4-dinitro-N-(4-dimethylaminobenzoyl)aniline and 2,3-dihydrobenzofuran-5-carboxaldehyde. [M+H]⁺ calcd for C₂₄H₂₄N₄O₂: 399.18. Found: 399.47.

### Example 84

### N-(2-(2,3-dihydrobenzo[b][1,4]dioxin-6-yl)-1H-benzo[d]imidazol-5-yl)-4-(dimethylamino)benzamide (Compound 184)

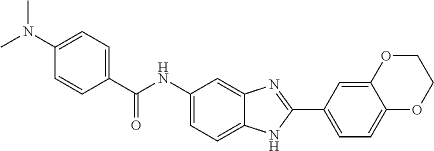 [SMILES: CN(C)c1ccc(C(=O)Nc2ccc3[nH]c(-c4ccc5c(c4)OCCO5)nc3c2)cc1]

Compound 184 was prepared according to the procedure similar to that described in Scheme III from 3,4-dinitro-N-(4-dimethylaminobenzoyl)aniline and benzo[1,4]dioxane-6-carboxaldehyde. [M+H]⁺ calcd for C₂₄H₂₂N₄O₃: 415.17. Found: 415.47.

### Example 85

### 4-(dimethylamino)-N-(2-(1-methyl-1H-benzo[d][1,2,3]triazol-5-yl)-1H-benzo[d]imidazol-5-yl)benzamide (Compound 185)

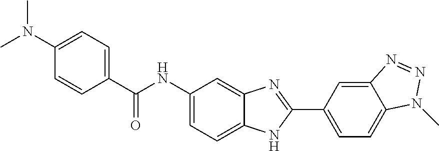

Compound 185 was prepared according to the procedure similar to that described in Scheme III from 3,4-dinitro-N-(4-dimethylaminobenzoyl)aniline and 1-methylbenzotriazole-5-carboxaldehyde. [M+H]⁺ calcd for C₂₃H₂₁N₇O: 412.18. Found: 412.50.

### Example 86

### N-(2-(benzo[c][1,2,5]oxadiazol-5-yl)-1H-benzo[d]imidazol-5-yl)-4-(dimethylamino)benzamide (Compound 186)

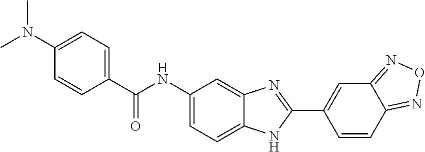 [SMILES: CN(C)c1ccc(C(=O)Nc2ccc3[nH]c(-c4ccc5nonc5c4)nc3c2)cc1]

Compound 186 was prepared according to the procedure similar to that described in Scheme III from 3,4-dinitro-N-(4-dimethylaminobenzoyl)aniline and benzofurazan-5-carboxaldehyde. [M+H]⁺ calcd for C₂₂H₈N₆O₂: 399.15. Found: 399.47.

### Example 87

### N-(1H, 1′H-[2,5′-bibenzo[d]imidazol]-5-yl)-4-(dimethylamino)benzamide (Compound 187)

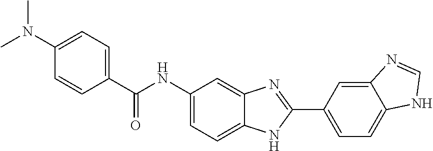 [SMILES: CN(C)c1ccc(C(=O)Nc2ccc3[nH]c(-c4ccc5[nH]cnc5c4)nc3c2)cc1]

Compound 187 was prepared according to the procedure similar to that described in Scheme III from 3,4-dinitro-N-(4-dimethylaminobenzoyl)aniline and benzimidazole-5-carboxaldehyde. [M+H]⁺ calcd for C₂₃H₂₀N₆O: 397.17. Found: 397.52.

### Example 88

### N-(2-(benzofuran-5-yl)-1H-benzo[d]imidazol-5-yl)-4-(dimethylamino)benzamide (Compound 188)

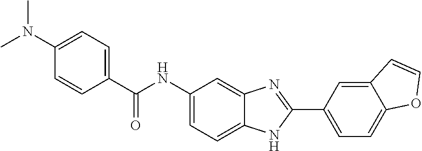 [SMILES: CN(C)c1ccc(C(=O)Nc2ccc3[nH]c(-c4ccc5occc5c4)nc3c2)cc1]

Compound 188 was prepared according to the procedure similar to that described in Scheme III from 3,4-dinitro-N-(4-dimethylaminobenzoyl)aniline and benzofuran-5-carboxaldehyde. [M+H]⁺ calcd for C₂₄H₂₀N₄O₂: 397.16. Found: 397.52.

### Example 89

### 2N-(2-(4-(1H-imidazol-1-yl)phenyl)-1H-benzo[d]imidazol-5-yl)-4-(pyrrolidin-1-yl)benzamide (Compound 189)

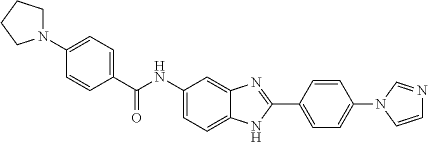 [SMILES: O=C(Nc1ccc2[nH]c(-c3ccc(-n4ccnc4)cc3)nc2c1)c1ccc(N2CCCC2)cc1]

Compound 189 was prepared according to the procedure similar to that described in Scheme III from 3,4-dinitro-N-(4-pyrrolidin-1-ylbenzoyl)aniline and 4-imidazolylbenzaldehyde. [M+H]⁺ calcd for C₂₇H₂₄N₆O: 449.21. Found: 449.48.

### Example 90

### N-(2-(4-(dimethylamino)phenyl)-1H-benzo[d]imidazol-5-yl)-1-methyl-1H-benzo[d][1,2,3]triazole-6-carboxamide (Compound 190)

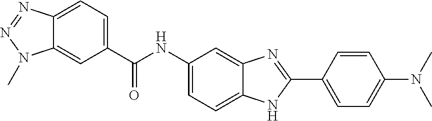 [SMILES: CN(C)c1ccc(-c2nc3cc(NC(=O)c4ccc5nnn(C)c5c4)ccc3[nH]2)cc1]

Compound 190 was prepared according to the procedure similar to that described in Scheme III from 3,4-dinitro-N-(1-methyl-6-benzotriazolylcarbonyl)aniline and 4-dimethylaminobenzaldehyde. ¹H NMR (500 MHz, DMSO-d₆) δ 10.74 (s, 1H), 8.78 (s, 1H), 8.43 (d, J=1.5 Hz, 1H), 8.16 (dd, J=1, 8.5 Hz, 1H), 8.10 (d, J=9 Hz, 2H), 8.01 (d, J=8.5 Hz, 2H), 7.84 (dd, J=2, 9 Hz, 1H), 7.72 (d, J=9 Hz, 1H), 6.96 (d, J=9 Hz, 2H), 4.39 (s, 3H), 3.10 (s, 6H).

### Example 91

### 4-(5-benzamido-1H-benzo[d]imidazol-2-yl)-N-(4-(dimethylamino)phenyl)benzamide (Compound 191)

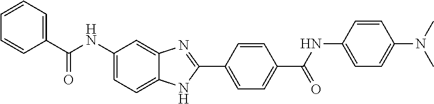 [SMILES: CN(C)c1ccc(NC(=O)c2ccc(-c3nc4cc(NC(=O)c5ccccc5)ccc4[nH]3)cc2)cc1]

Compound 191 was prepared according to the procedure similar to that described in Scheme III from 3,4-dinitro-N-(4-dimethylaminobenzoyl)aniline and 4-phenylaminocarbonylbenzaldehyde. [M+H]⁺ calcd for C₂₉H₂₅N₅O₂: 476.20. Found: 475.98.

### Example 92

### 4-(dimethylamino)-N-(2-(4-(phenylcarbamoyl)phenyl)-1H-benzo[d]imidazol-5-yl)benzamide (Compound 192)

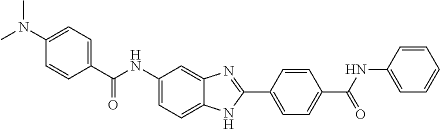 [SMILES: CN(C)c1ccc(C(=O)Nc2ccc3[nH]c(-c4ccc(C(=O)Nc5ccccc5)cc4)nc3c2)cc1]

Compound 192 was prepared according to the procedure similar to that described in Scheme III from 3,4-dinitro-N-benzoylaniline and 4-(4-dimethylaminophenyl)aminocarbonylbenzaldehyde. [M+H]⁺ calcd for C₂₉H₂₅N₅O₂: 476.20. Found: 475.91.

### Example 93

### 4-(dimethylamino)-N-(2-(4-((4-(dimethylamino)phenyl)carbamoyl)phenyl)-1H-benzo[d]imidazol-5-yl)benzamide (Compound 193)

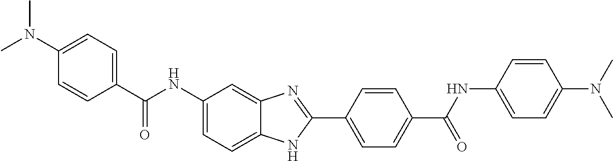

Compound 193 was prepared according to the procedure similar to that described in Scheme III from 3,4-dinitro-N-(4-dimethylaminobenzoyl)aniline and 4-(4-dimethylaminophenyl)aminocarbonylbenzaldehyde. [M+H]⁺ calcd for C₃₁H₃₀N₆O₂: 519.24. Found: 519.04.

### Example 94

### 3-chloro-N-(2-(4-(phenylcarbamoyl)phenyl)-1H-benzo[d]imidazol-5-yl)benzamide (Compound 194)

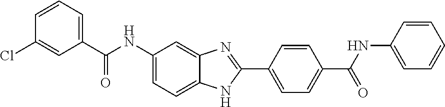 [SMILES: O=C(Nc1ccccc1)c1ccc(-c2nc3cc(NC(=O)c4cccc(Cl)c4)ccc3[nH]2)cc1]

Compound 194 was prepared according to the procedure similar to that described in Scheme III from 3,4-dinitro-N-(4-chlorobenzoyl)aniline and 4-phenylaminocarbonylbenzaldehyde. [M+H]⁺ calcd for C₂₇H₁₉ClN₄O₂: 467.12. Found: 466.93.

### Example 95

### 4-morpholino-N-(2-(4-((4-morpholinophenyl)carbamoyl)phenyl)-1H-benzo[d]imidazol-5-yl)benzamide (Compound 195)

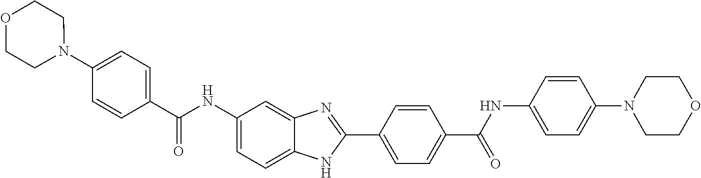 [SMILES: O=C(Nc1ccc(N2CCOCC2)cc1)c1ccc(-c2nc3cc(NC(=O)c4ccc(N5CCOCC5)cc4)ccc3[nH]2)cc1]

Compound 195 was prepared according to the procedure similar to that described in Scheme III from 3,4-dinitro-N-(4-morpholin-4-ylbenzoyl)aniline and 4-(4-morpholinylphenyl)aminocarbonylbenzaldehyde. [M+H]⁺ calcd for C₃₅H₃₄N₆O₄: 603.26. Found: 603.36.

### Example 96

### 4-(4-methylpiperazin-1-yl)-N-(2-(4-((4-(4-methylpiperazin-1-yl)phenyl)carbamoyl)phenyl)-1H-benzo[d]imidazol-5-yl)benzamide (Compound 196)

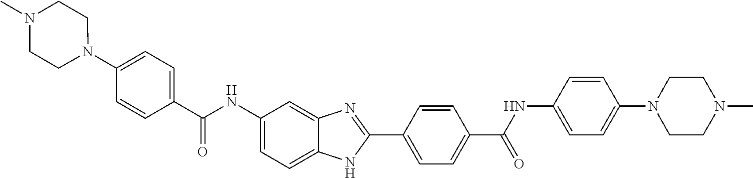

Compound 196 was prepared according to the procedure similar to that described in Scheme III from 3,4-dinitro-N-(4-(4-methylpiperazin-1-yl)benzoyl)aniline and 4-(4-(4-methylpiperazin-1-yl)phenyl)aminocarbonylbenzaldehyde. [M+H]⁺ calcd for C₃₇H₄₀N₈O₂: 629.33. Found: 629.16.

### Example 97

### N-(4-(dimethylamino)phenyl)-2-(4-((4-(dimethylamino)phenyl)carbamoyl)phenyl)-1H-benzo[d]imidazole-5-carboxamide (Compound 197)

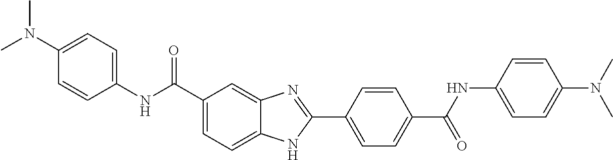

Compound 197 was prepared according to the procedure similar to that described in Scheme III from N-(4-dimethylaminophenyl)-3,4-dinitrobenzamide and 4-(4-dimethylaminophenyl)aminocarbonylbenzaldehyde. [M+H]⁺ calcd for C₃₁H₃₀N₆O₂: 519.24. Found: 519.04.

### Example 98

### 2-(4-(4-(dimethylamino)benzamido)phenyl)-N-(4-(dimethylamino)phenyl)-1H-benzo[d]imidazole-5-carboxamide (Compound 198)

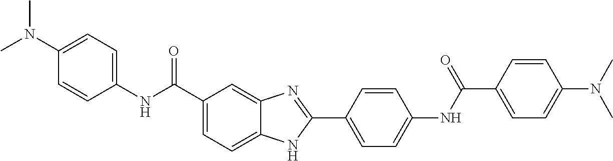

Compound 198 was prepared according to the procedure similar to that described in Scheme III from N-(4-dimethylaminophenyl)-3,4-dinitrobenzamide and 4-(4-dimethylaminobenz)amidobenzaldehyde. [M+H]calcd for C₃₁H₃₀N₆O₂: 519.24. Found: 518.97.

### Example 99

### N-(2-(4-(1H-pyrazol-1-yl)phenyl)-1H-benzo[d]imidazol-6-yl)-4-(dimethylamino)benzamide (Compound 199)

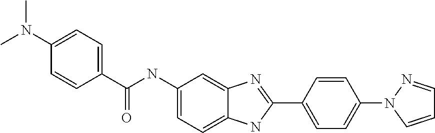 [SMILES: CN(C)c1ccc(C(=O)Nc2ccc3[nH]c(-c4ccc(-n5cccn5)cc4)nc3c2)cc1]

Compound 199 was prepared according to the procedure similar to that described in Scheme III from 3,4-dinitro-N-(4-dimethylaminobenzoyl)aniline and 4-imidazolylbenzaldehyde. [M+H]⁺ calcd for C₂₅H₂₂N₆O: 423.20. Found: 423.01.

### Example 100

### N-(2-(4-(1H-1,2,4-triazol-1-yl)phenyl)-1H-benzo[d]imidazol-5-yl)-4-(dimethylamino)benzamide (Compound 200)

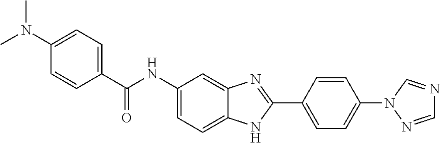 [SMILES: CN(C)c1ccc(C(=O)Nc2ccc3[nH]c(-c4ccc(-n5cncn5)cc4)nc3c2)cc1]

Compound 200 was prepared according to the procedure similar to that described in Scheme III from 3,4-dinitro-N-(4-dimethylaminobenzoyl)aniline and 4-(1,2,4-triazolyl-1-)benzaldehyde. [M+H]⁺ calcd for C₂₄H₂₁N₇O: 424.19. Found: 424.02.

### Example 101

### N-(2-phenyl-1H-benzo[d]imidazol-5-yl)benzo[b]thiophene-2-carboxamide (Compound 201)

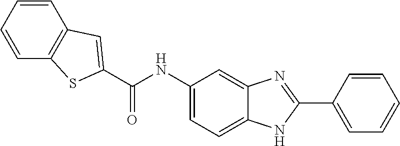 [SMILES: O=C(Nc1ccc2[nH]c(-c3ccccc3)nc2c1)c1cc2ccccc2s1]

Compound 201 was prepared according to the procedure similar to that described in Scheme III from N-(3,4-dinitrophenyl)benzo[b]thiophene-2-carboxamide and benzaldehyde. ¹H NMR (500 MHz, CD₃OD) δ 8.18 (d, J=2 Hz, 1H), 8.14 (s, 1H), 8.06 (d, J=8 Hz, 2H), 7.91 (m, 2H), 7.85 (d, J=8.5 Hz, 1H), 7.73 (d, J=8.5 Hz, 1H), 7.60 (d, J=8.5 Hz, 1H), 7.53 (m, 3H), 7.44 (m, 3H).

### Example 102

### N-(2-(4-(2H-tetrazol-5-yl)phenyl)-1H-benzo[d]imidazol-5-yl)-4-(dimethylamino)benzamide (Compound 202)

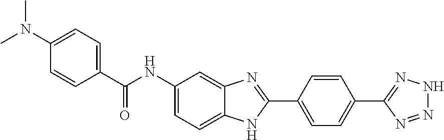 [SMILES: CN(C)c1ccc(C(=O)Nc2ccc3[nH]c(-c4ccc(-c5nn[nH]n5)cc4)nc3c2)cc1]

Compound 202 was prepared according to the procedure similar to that described in Scheme III from 3,4-dinitro-N-(4-dimethylaminobenzoyl)aniline and 4-(1,2,3,5-tetrazolyl-5-)benzaldehyde. [M+H]⁺ calcd for C₂₃H₂₀N₈O: 415.19. Found: 424.97.

### Example 103

### N-(2-phenyl-1H-benzo[d]imidazol-5-yl)-1H-indole-2-carboxamide (Compound 203)

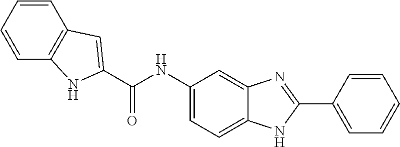 [SMILES: O=C(Nc1ccc2[nH]c(-c3ccccc3)nc2c1)c1cc2ccccc2[nH]1]

Compound 203 was prepared according to the procedure similar to that described in Scheme III from 3,4-dinitro-N-(indol-2-oyl)aniline and benzaldehyde. ¹H NMR (500 MHz, CD₃OD) δ 8.20 (d, J=1.5 Hz, 1H), 8.09 (dd, J=1.5, 8 Hz, 2H). 7.66 (d, J=8 Hz, 1H), 7.61 (d, J=8.5 Hz, 1H), 7.57-7.52 (m, 4H), 7.48 (dd, J=0.5, 8 Hz, 1H), 7.34 (s, 1H), 7.25 (dt, J=1, 7 Hz, 1H), 7.09 (dt, J=0.5, 8 Hz, 1H).

### Example 104

### N-(2-phenyl-1H-benzo[d]imidazol-5-yl)-1,2,3,4-tetrahydronaphthalene-2-carboxamide (Compound 204)

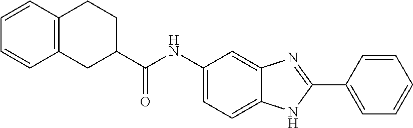 [SMILES: O=C(Nc1ccc2[nH]c(-c3ccccc3)nc2c1)C1CCc2ccccc2C1]

Compound 204 was prepared according to the procedure similar to that described in Scheme III from 3,4-dinitro-N-(1,2,3,4-tetrahydronaphthalen-2-oyl)aniline and benzaldehyde. [M+H]⁺ calcd for C₂₄H₂₁N₃O: 368.17. Found: 368.00.

### Example 105

### N-(2-(4-methoxyphenyl)-1H-benzo[d]imidazol-5-yl)isonicotinamide (Compound 205)

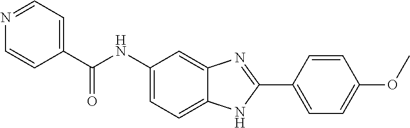 [SMILES: COc1ccc(-c2nc3cc(NC(=O)c4ccncc4)ccc3[nH]2)cc1]

Compound 205 was prepared according to the procedure similar to that described in Scheme III from 3,4-dinitro-N-(pyridin-4-oyl)aniline and 4-methoxybenzaldehyde. [M+H]⁺ calcd for C₂₀H₁₆N₄O₂: 345.13. Found: 345.00.

### Example 106

### 4-(dimethylamino)-N-(2-(4-(dimethylamino)phenyl)-1H-benzo[d]imidazol-5-yl)benzamide (Compound 206)

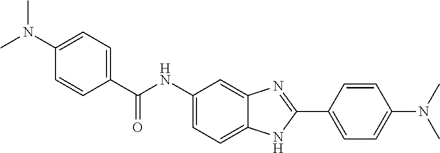

Compound 206 was prepared according to the procedure similar to that described in Scheme III from 3,4-dinitro-N-(4-dimethylaminobenzoyl)aniline and 4-dimethylaminobenzaldehyde. ¹H NMR (300 MHz, DMSO-d₆) δ 10.20 (s, 1H), 8.39 (s, 1H), 8.11 (d, J=8 Hz, 2H), 7.90 (d, J=9 Hz, 2H), 7.81 (d, J=9 Hz, 2H), 7.68 (d, J=9 Hz, 2H), 6.95 (d, J=9 Hz, 2H), 6.78 (d, J=9 Hz, 2H), 3.07 (s, 6H), 2.99 (s, 6H).

### Example 107

### N-(2-(4-(dimethylamino)phenyl)-1H-benzo[d]imidazol-5-yl)isonicotinamide (Compound 207)

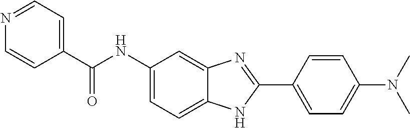 [SMILES: CN(C)c1ccc(-c2nc3cc(NC(=O)c4ccncc4)ccc3[nH]2)cc1]

Compound 207 was prepared according to the procedure similar to that described in Scheme III from 3,4-dinitro-N-(pyridin-4-oyl)aniline and 4-dimethylaminobenzaldehyde. ¹H NMR (500 MHz, DMSO-d₆) δ 10.90 (s, 1H), 8.83 (d, J=6 Hz, 2H), 8.40 (s, 1H), 8.10 (d, J=9 Hz, 2H), 7.98 (d, J=6 Hz, 2H), 7.81 (dd, J=2, 9 Hz, 1H), 7.75 (d, J=9 Hz, 1H), 6.97 (d, J=9 Hz, 2H), 3.09 (s, 6H).

### Example 108

### N-(2-(4-(dimethylamino)phenyl)-1H-benzo[d]imidazol-5-yl)-4-methoxybenzamide (Compound 208)

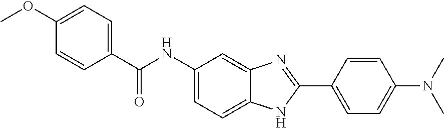

Compound 208 was prepared according to the procedure similar to that described in Scheme III from 3,4-dinitro-N-(4-methoxybenzoyl)aniline and 4-dimethylaminobenzaldehyde. ¹H NMR (500 MHz, DMSO-d₆) δ 10.41 (s, 1H), 8.39 (d, J=1.5 Hz, 1H), 8.08 (d, J=9.5 Hz, 2H), 8.01 (d, J=7 Hz, 2H), 7.79 (dd, J=2, 9 Hz, 1H), 7.67 (d, J=9 Hz, 1H), 7.09 (d, J=9 Hz, 2H), 6.96 (d, J=9 Hz, 2H), 3.82 (s, 3H), 3.08 (s, 6H).

### Example 109

### 4-(1H-imidazol-1-yl)-N-(2-(4-methoxyphenyl)-1H-benzo[d]imidazol-5-yl)benzamide (Compound 209)

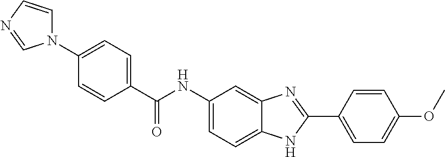 [SMILES: COc1ccc(-c2nc3cc(NC(=O)c4ccc(-n5ccnc5)cc4)ccc3[nH]2)cc1]

Compound 209 was prepared according to the procedure similar to that described in Scheme III from 3,4-dinitro-N-(4-imidazol-1-ylbenzoyl)aniline and 4-methoxybenzaldehyde. [M+H]⁺ calcd for C₂₄H₁₉N₅O₂: 410.05. Found: 410.00.

### Example 110

### N-(2-(4-methoxyphenyl)-1H-benzo[d]imidazol-5-yl)thiophene-2-carboxamide (Compound 210)

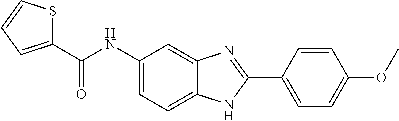 [SMILES: COc1ccc(-c2nc3cc(NC(=O)c4cccs4)ccc3[nH]2)cc1]

Compound 210 was prepared according to the procedure similar to that described in Scheme III from 3,4-dinitro-N-(thien-2-oyl)aniline and 4-methoxybenzaldehyde. [M+H]⁺ calcd for C₁₉H₁₅N₃O₂S: 350.09. Found: 349.89.

### Example 111

### N,2-bis(4-morpholinophenyl)-1H-benzo[d]imidazole-5-carboxamide (Compound 211)

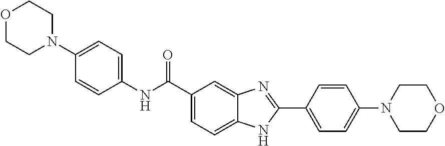 [SMILES: O=C(Nc1ccc(N2CCOCC2)cc1)c1ccc2[nH]c(-c3ccc(N4CCOCC4)cc3)nc2c1]

Compound 211 was prepared according to the procedure similar to that described in Scheme III from N-(4-morpholinylphenyl)-3,4-dinitrobenzamide and 4-morpholinylbenzaldehyde. [M+H]⁺ calcd for C₂₈H₂₉N₅O₃: 484.23. Found: 483.92.

### Example 112

### 4-morpholino-N-(2-(4-morpholinophenyl)-1H-benzo[d]imidazol-5-yl)benzamide (Compound 212)

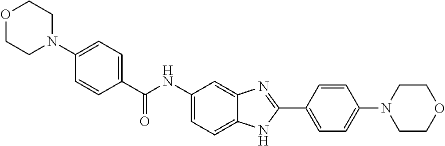 [SMILES: O=C(Nc1ccc2[nH]c(-c3ccc(N4CCOCC4)cc3)nc2c1)c1ccc(N2CCOCC2)cc1]

Compound 212 was prepared according to the procedure similar to that described in Scheme III from 3,4-dinitro-N-(4-morpholin-4-ylbenzoyl)aniline and 4-morpholinylbenzaldehyde. [M+H]⁺ calcd for C₂₈H₂₉N₅O₃: 484.23. Found: 483.94.

### Example 113

### N-phenyl-4-(5-(4-(pyrrolidin-1-yl)benzamido)-1H-benzo[d]imidazol-2-yl)benzamide (Compound 213)

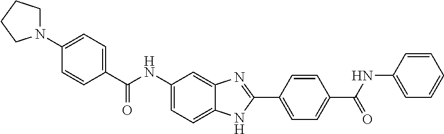 [SMILES: O=C(Nc1ccccc1)c1ccc(-c2nc3cc(NC(=O)c4ccc(N5CCCC5)cc4)ccc3[nH]2)cc1]

Compound 213 was prepared according to the procedure similar to that described in Scheme III from N-(3,4-dinitrophenyl)-4-pyrrolidinylbenzamide and 4-phenylaminocarbonylbenzaldehyde. [M+H]⁺ calcd for C₃₁H₂₇N₅O₂: 502.23. Found: 502.03.

### Example 114

### 4-((2-hydroxyethyl)(methyl)amino)-N-(2-(4-(phenylcarbamoyl)phenyl)-1H-benzo[d]imidazol-5-yl)benzamide (Compound 214)

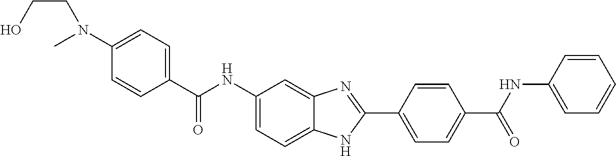 [SMILES: CN(CCO)c1ccc(C(=O)Nc2ccc3[nH]c(-c4ccc(C(=O)Nc5ccccc5)cc4)nc3c2)cc1]

Compound 214 was prepared according to the procedure similar to that described in Scheme III from N-(3,4-dinitrophenyl)-4-(N-methyl-N-2-hydroxyethylamino)benzamide and 4-phenylaminocarbonylbenzaldehyde. [M+H]⁺ calcd for C₃₀H₂₇N₅O₃: 506.22. Found: 506.01

### Example 115

### N-phenyl-4-(5-(4-(piperidin-1-yl)benzamido)-1H-benzo[d]imidazol-2-yl)benzamide (Compound 215)

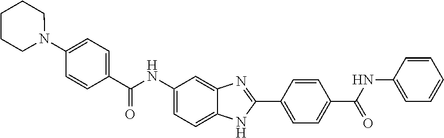 [SMILES: O=C(Nc1ccccc1)c1ccc(-c2nc3cc(NC(=O)c4ccc(N5CCCCC5)cc4)ccc3[nH]2)cc1]

Compound 215 was prepared according to the procedure similar to that described in Scheme III from N-(3,4-dinitrophenyl)-4-piperidinylbenzamide and 4-phenylaminocarbonylbenzaldehyde. [M+H]⁺ calcd for C₃₂H₂₉N₅O₂: 516.24. Found: 516.07.

### Example 116

### 4-(4-methylpiperazin-1-yl)-N-(2-(4-(phenylcarbamoyl)phenyl)-1H-benzo[d]imidazol-5-yl)benzamide (Compound 216)

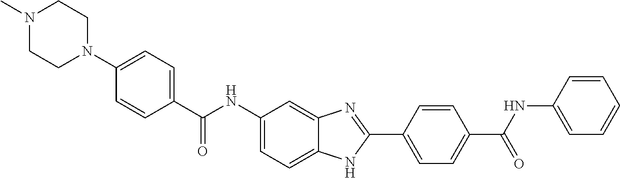 [SMILES: CN1CCN(c2ccc(C(=O)Nc3ccc4[nH]c(-c5ccc(C(=O)Nc6ccccc6)cc5)nc4c3)cc2)CC1]

Compound 216 was prepared according to the procedure similar to that described in Scheme III from N-(3,4-dinitrophenyl)-4-(1-methyl-4-piperazinyl)benzamide and 4-phenylaminocarbonylbenzaldehyde. [M+H]⁺ calcd for C₃₂H₃₀N₆O₂: 531.25. Found: 531.05.

### Example 117

### 4-morpholino-N-(2-(4-(phenylcarbamoyl)phenyl)-1H-benzo[d]imidazol-5-yl)benzamide (Compound 217)

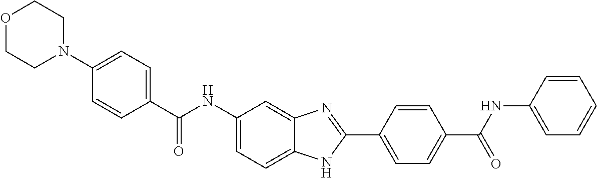 [SMILES: O=C(Nc1ccccc1)c1ccc(-c2nc3cc(NC(=O)c4ccc(N5CCOCC5)cc4)ccc3[nH]2)cc1]

Compound 217 was prepared according to the procedure similar to that described in Scheme III from N-(3,4-dinitrophenyl)-4-morpholinylbenzamide and 4-phenylaminocarbonylbenzaldehyde. [M+H]⁺ calcd for C₃₁H₂₇N₅O₃: 518.22. Found: 518.03.

### Example 118

### 4-(dimethylamino)-N-(2-(4-(2-hydroxyethoxy)phenyl)-1H-benzo[d]imidazol-5-yl)benzamide (Compound 218)

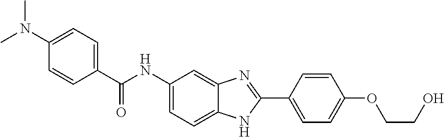 [SMILES: CN(C)c1ccc(C(=O)Nc2ccc3[nH]c(-c4ccc(OCCO)cc4)nc3c2)cc1]

Compound 218 was prepared according to the procedure similar to that described in Scheme III from N-(3,4-dinitrophenyl)-4-dimethylaminobenzamide and 4-(2-hydroxyethyloxy)benzaldehyde. [M+H]⁺ calcd for C₂₄H₂₄N₄O₃: 417.19. Found: 417.00.

### Example 119

### 4-(dimethylamino)-N-(2-(3-(2-hydroxyethoxy)phenyl)-1H-benzo[d]imidazol-5-yl)benzamide (Compound 219)

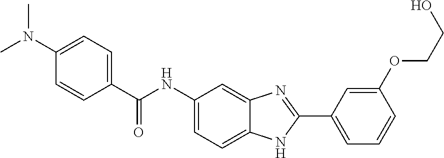 [SMILES: CN(C)c1ccc(C(=O)Nc2ccc3[nH]c(-c4cccc(OCCO)c4)nc3c2)cc1]

Compound 219 was prepared according to the procedure similar to that described in Scheme III from N-(3,4-dinitrophenyl)-4-dimethylaminobenzamide and 3-(2-hydroxyethyloxy)benzaldehyde. [M+H]⁺ calcd for C₂₄H₂₄N₄O₃: 417.19. Found: 416.94.

### Example 120

### 4-(dimethylamino)-N-(2-(4′-(dimethylamino)-[1,1′-biphenyl]-4-yl)-1H-benzo[d]imidazol-5-yl)benzamide (Compound 220)

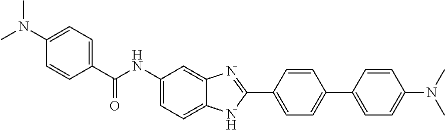

Compound 220 was prepared according to the procedure similar to that described in Scheme III from N-(3,4-dinitrophenyl)-4-dimethylaminobenzamide and 4-(4-dimethylaminophenyl)benzaldehyde. [M+H]⁺ calcd for C₃₀H₂₉N₅O: 476.24. Found: 475.98.

### Example 121

### N-(2-(4-(phenylcarbamoyl)phenyl)-1H-benzo[d]imidazol-5-yl)-2-(pyrrolidin-1-yl)pyrimidine-5-carboxamide (Compound 221)

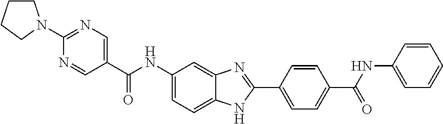 [SMILES: O=C(Nc1ccccc1)c1ccc(-c2nc3cc(NC(=O)c4cnc(N5CCCC5)nc4)ccc3[nH]2)cc1]

Compound 221 was prepared according to the procedure similar to that described in Scheme III from N-(3,4-dinitrophenyl)-2-(pyrrolidin-1-yl)pyrimidine-5-carboxamide and 4-phenylaminocarbonylbenzaldehyde. [M+H]⁺ calcd for C₂₉H₂₅N₇O₂: 504.22. Found: 503.92.

### Example 122

### 4-(5-(4-(1H-tetrazol-1-yl)benzamido)-1H-benzo[d]imidazol-2-yl)-N-phenylbenzamide (Compound 222)

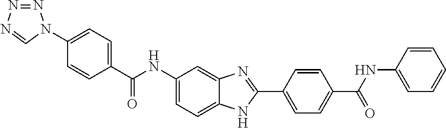 [SMILES: O=C(Nc1ccccc1)c1ccc(-c2nc3cc(NC(=O)c4ccc(-n5cnnn5)cc4)ccc3[nH]2)cc1]

Compound 222 was prepared according to the procedure similar to that described in Scheme III from N-(3,4-dinitrophenyl)-4-tetrazolylbenzamide and 4-phenylaminocarbonylbenzaldehyde. [M+H]⁺ calcd for C₂₈H₂₀N₈O₂: 501.18. Found: 500.88.

### Example 123

### N-(2-(4-(2-hydroxyethoxy)phenyl)-1H-benzo[d]imidazol-5-yl)-4-((2-hydroxyethyl)(methyl)amino)benzamide (Compound 223)

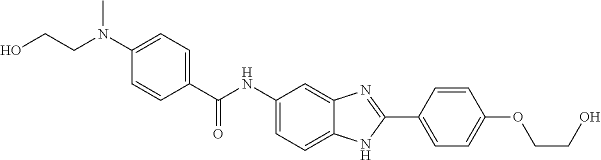 [SMILES: CN(CCO)c1ccc(C(=O)Nc2ccc3[nH]c(-c4ccc(OCCO)cc4)nc3c2)cc1]

Compound 223 was prepared according to the procedure similar to that described in Scheme III from N-(3,4-dinitrophenyl)-4-(N-methyl-N-2-hydroxyethylamino)benzamide and 4-(2-hydroxyethyloxy)benzaldehyde. [M+H]⁺ calcd for C₂₅H₂₆N₄O₄: 447.21. Found: 446.89.

### Example 124

### 3-(5-(4-(dimethylamino)benzamido)-1H-benzo[d]imidazol-2-yl)-N-(4-(dimethylamino)phenyl)benzamide (Compound 224)

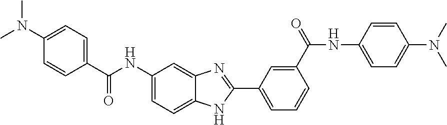

Compound 224 was prepared according to the procedure similar to that described in Scheme III from 3,4-dinitro-N-(4-dimethylaminobenzo)aniline and 3-(4-dimethylaminophenyl)aminocarbonylbenzaldehyde. [M+H]⁺ calcd for C₃₁H₃₀N₆O₂: 519.25. Found: 519.04.

### Example 125

### 4-(pyrrolidin-1-yl)-N-(2-(4-(pyrrolidin-1-yl)phenyl)-1H-benzo[d]imidazol-5-yl)benzamide (Compound 225)

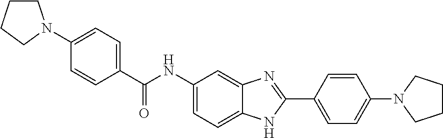 [SMILES: O=C(Nc1ccc2[nH]c(-c3ccc(N4CCCC4)cc3)nc2c1)c1ccc(N2CCCC2)cc1]

Compound 225 was prepared according to the procedure similar to that described in Scheme III from N-(3,4-dinitrophenyl)-4-pyrrolidinylbenzamide and 4-pyrrolidinylbenzaldehyde. [M+H]⁺ calcd for C₂₈H₂₉N₅O: 452.25. Found: 451.95.

### Example 126

### N-(2-(4-(pyrrolidin-1-yl)phenyl)-1H-benzo[d]imidazol-5-yl)-4-(1H-tetrazol-1-yl)benzamide (Compound 226)

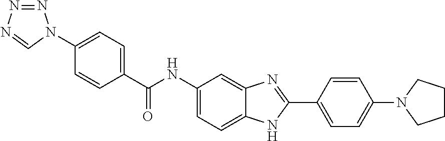 [SMILES: O=C(Nc1ccc2[nH]c(-c3ccc(N4CCCC4)cc3)nc2c1)c1ccc(-n2cnnn2)cc1]

Compound 226 was prepared according to the procedure similar to that described in Scheme III from N-(3,4-dinitrophenyl)-4-tetrazolylbenzamide and 4-pyrrolidinylbenzaldehyde. [M+H]⁺ calcd for C₂₅H₂₂N₈O: 451.20. Found: 451.50.

### Example 127

### N-(2-hydroxyethyl)-4-(5-(4-(pyrrolidin-1-yl)benzamido)-1H-benzo[d]imidazol-2-yl)benzamide (Compound 227)

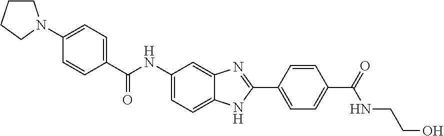 [SMILES: O=C(NCCO)c1ccc(-c2nc3cc(NC(=O)c4ccc(N5CCCC5)cc4)ccc3[nH]2)cc1]

Compound 227 was prepared according to the procedure similar to that described in Scheme III from N-(3,4-dinitrophenyl)-4-pyrrolidinylbenzamide and 4-(2-hydroxyethyl)aminocarbonylbenzaldehyde. [M+H]⁺ calcd for C₂₇H₂₇N₅O₃: 470.22. Found: 469.58.

### Example 128

### N-cyclopropyl-4-(5-(4-(pyrrolidin-1-yl)benzamido)-1H-benzo[d]imidazol-2-yl)benzamide (Compound 228)

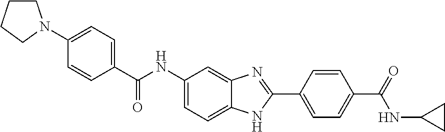 [SMILES: O=C(Nc1ccc2[nH]c(-c3ccc(C(=O)NC4CC4)cc3)nc2c1)c1ccc(N2CCCC2)cc1]

Compound 228 was prepared according to the procedure similar to that described in Scheme III from N-(3,4-dinitrophenyl)-4-pyrrolidinylbenzamide and 4-cyclopropylaminocarbonylbenzaldehyde. [M+H]⁺ calcd for C₂₈H₂₇N₅O₂: 466.23. Found: 466.55.

### Example 129

### N-(2,3-dihydroxypropyl)-4-(5-(4-(pyrrolidin-1-yl)benzamido)-1H-benzo[d]imidazol-2-yl)benzamide (Compound 229)

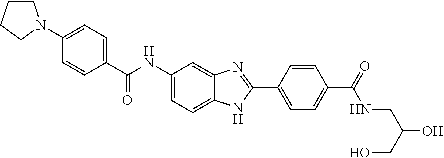 [SMILES: O=C(NCC(O)CO)c1ccc(-c2nc3cc(NC(=O)c4ccc(N5CCCC5)cc4)ccc3[nH]2)cc1]

Compound 229 was prepared according to the procedure similar to that described in Scheme III from N-(3,4-dinitrophenyl)-4-pyrrolidinylbenzamide and 4-(2,3-dihydroxypropyl)aminocarbonylbenzaldehyde. [M+H]⁺ calcd for C₂₈H₂₉N₅O₄: 500.23. Found: 500.56.

### Example 130

### N-(2-(4-(hydrazinecarbonyl)phenyl)-1H-benzo[d]imidazol-5-yl)-4-(pyrrolidin-1-yl)benzamide (Compound 230)

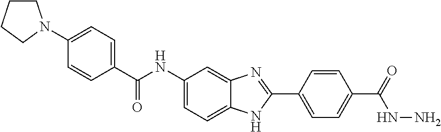 [SMILES: NNC(=O)c1ccc(-c2nc3cc(NC(=O)c4ccc(N5CCCC5)cc4)ccc3[nH]2)cc1]

Compound 230 was prepared according to the procedure similar to that described in Scheme III from N-(3,4-dinitrophenyl)-4-pyrrolidinylbenzamide and 4-hydrazinylcarbonylbenzaldehyde. [M+H]⁺ calcd for C₂₅H₂₄N₆O₂: 441.21. Found: 441.51.

### Example 131

### N-(2-morpholinoethyl)-4-(5-(4-(pyrrolidin-1-yl)benzamido)-1H-benzo[d]imidazol-2-yl)benzamide (Compound 231)

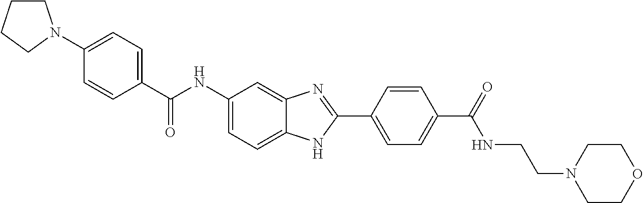 [SMILES: O=C(NCCN1CCOCC1)c1ccc(-c2nc3cc(NC(=O)c4ccc(N5CCCC5)cc4)ccc3[nH]2)cc1]

Compound 231 was prepared according to the procedure similar to that described in Scheme III from N-(3,4-dinitrophenyl)-4-pyrrolidinylbenzamide and 4-(2-morpholinylethyl)aminocarbonylbenzaldehyde. [M+H]⁺ calcd for C₃₁H₃₄N₆O₃: 539.28. Found: 539.63.

### Example 132

### N-(3-methyl-1H-pyrazol-4-yl)-4-(5-(4-(pyrrolidin-1-yl)benzamido)-1H-benzo[d]imidazol-2-yl)benzamide (Compound 232)

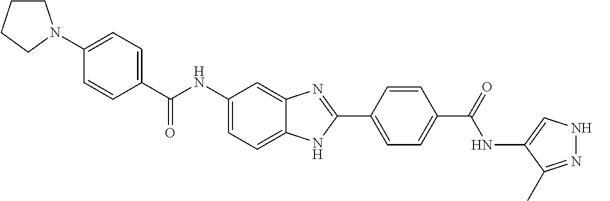 [SMILES: Cc1n[nH]cc1NC(=O)c1ccc(-c2nc3cc(NC(=O)c4ccc(N5CCCC5)cc4)ccc3[nH]2)cc1]

Compound 232 was prepared according to the procedure similar to that described in Scheme III from N-(3,4-dinitrophenyl)-4-pyrrolidinylbenzamide and 4-(3-methyl-4-pyrazolyl)aminocarbonylbenzaldehyde. [M+H]⁺ calcd for C₂₉H₂₇N₇O₂: 506.23. Found: 506.25.

### Example 133

### N-(2-(diethylamino)ethyl)-2-(4-((4-(dimethylamino)phenyl)carbamoyl)phenyl)-1H-benzo[d]imidazole-5-carboxamide (Compound 233)

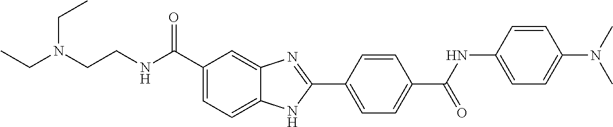 [SMILES: CCN(CC)CCNC(=O)c1ccc2[nH]c(-c3ccc(C(=O)Nc4ccc(N(C)C)cc4)cc3)nc2c1]

Compound 233 was prepared according to the procedure similar to that described in Scheme III from N-(2-N,N-diethylaminoethyl)-dinitrophenyl)-3,4-dinitrobenzamide and 4-(4-dimethylaminophenyl)aminocarbonylbenzaldehyde. [M+H]⁺ calcd for C₂₉H₃₄N₆O₂: 499.27. Found: 499.61.

### Example 134

### N-cyclopropyl-4-(5-(4-fluorobenzamido)-1H-benzo[d]imidazol-2-yl)benzamide (Compound 234)

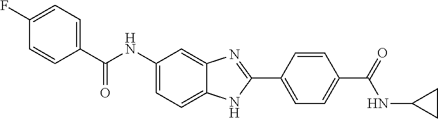 [SMILES: O=C(Nc1ccc2[nH]c(-c3ccc(C(=O)NC4CC4)cc3)nc2c1)c1ccc(F)cc1]

Compound 234 was prepared according to the procedure similar to that described in Scheme III from N-(3,4-dinitrophenyl)-4-fluorobenzamide and 4-cyclopropylaminocarbonylbenzaldehyde. [M+H]⁺ calcd for C₂₄H₁₉FN₄O₂: 415.16. Found: 415.53.

### Example 135

### N-cyclopropyl-4-(5-(4-((2-hydroxyethyl)(methyl)amino)benzamido)-1H-benzo[d]imidazol-2-yl)benzamide (Compound 235)

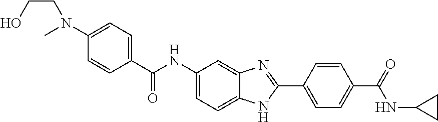 [SMILES: CN(CCO)c1ccc(C(=O)Nc2ccc3[nH]c(-c4ccc(C(=O)NC5CC5)cc4)nc3c2)cc1]

Compound 235 was prepared according to the procedure similar to that described in Scheme III from N-(3,4-dinitrophenyl)-4-(N-2-hydroxyethyl-N-methylamino)benzamide and 4-cyclopropylaminocarbonylbenzaldehyde. [M+H]⁺ calcd for C₂₇H₂₇N₅O₃: 470.22. Found: 470.60.

### Example 136

### N-cyclopropyl-4-(5-(4-((N-methylsulfamoyl)amino)benzamido)-1H-benzo[d]imidazol-2-yl)benzamide (Compound 236)

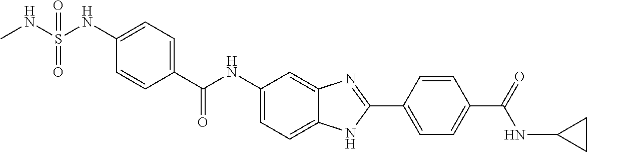 [SMILES: CNS(=O)(=O)Nc1ccc(C(=O)Nc2ccc3[nH]c(-c4ccc(C(=O)NC5CC5)cc4)nc3c2)cc1]

Compound 236 was prepared according to the procedure similar to that described in Scheme III from N-(3,4-dinitrophenyl)-4-methylaminosulfonamido)benzamide and 4-cyclopropylaminocarbonylbenzaldehyde. [M+H]⁺ calcd for C₂₅H₂₄N₆O₄S: 505.17. Found: 504.47.

### Example 137

### N-cyclopropyl-4-(5-(4-(2-hydroxyethoxy)benzamido)-1H-benzo[d]imidazol-2-yl)benzamide (Compound 237)

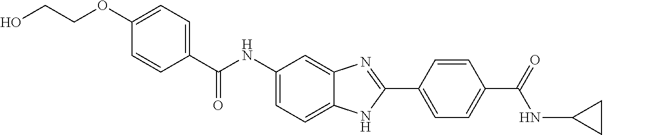 [SMILES: O=C(Nc1ccc2[nH]c(-c3ccc(C(=O)NC4CC4)cc3)nc2c1)c1ccc(OCCO)cc1]

Compound 237 was prepared according to the procedure similar to that described in Scheme III from N-(3,4-dinitrophenyl)-4-(2-hydroxyethoxy)benzamide and 4-cyclopropylaminocarbonylbenzaldehyde. [M+H]⁺ calcd for C₂₆H₂₄N₄O₄: 457.19. Found: 457.51.

### Example 138

### (E)-4-(dimethylamino)-N-(2-(3-((2-(4-(dimethylamino)benzoyl)hydrazono)methyl)phenyl)-1H-benzo[d]imidazol-5-yl)benzamide (Compound 238)

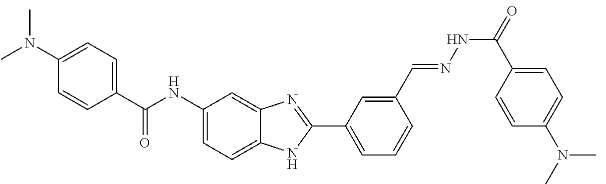

Compound 238 was prepared according to the procedure described in Scheme I from 4-dimethylaminobenzohydrazide and 3-(5-(4-dimethylaminobenzamido)benzimidazolyl-2-)benzaldehyde. [M+H]calcd for C₃₂H₃₁N₇O₂: 546.26. Found: 546.65.

### Example 139

### N-(2-(4-(morpholine-4-carbonyl)phenyl)-1H-benzo[d]imidazol-5-yl)-4-(pyrrolidin-1-yl)benzamide (Compound 239)

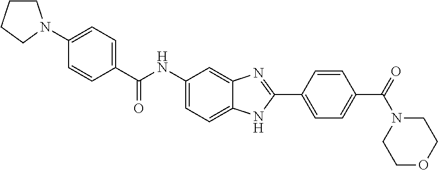 [SMILES: O=C(Nc1ccc2[nH]c(-c3ccc(C(=O)N4CCOCC4)cc3)nc2c1)c1ccc(N2CCCC2)cc1]

Compound 239 was prepared according to the procedure similar to that described in Scheme III from N-(3,4-dinitrophenyl)-4-pyrrolidinylbenzamide and 4-morpholinylcarbonylbenzaldehyde. [M+H]⁺ calcd for C₂₉H₂₉N₅O₃: 496.24. Found: 496.25.

### Example 140

### N-cyclopropyl-4-(5-(4-(3-hydroxypyrrolidin-1-yl)benzamido)-1H-benzo[d]imidazol-2-yl)benzamide (Compound 240)

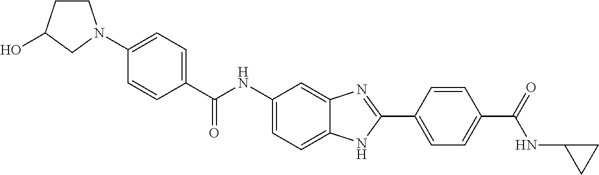 [SMILES: O=C(Nc1ccc2[nH]c(-c3ccc(C(=O)NC4CC4)cc3)nc2c1)c1ccc(N2CCC(O)C2)cc1]

Compound 240 was prepared according to the procedure similar to that described in Scheme III from N-(3,4-dinitrophenyl)-4-(3-hydroxypyrrolidinyl)benzamide and 4-cyclopropyl aminocarbonylbenzaldehyde. [M+H]⁺ calcd for C₂₈H₂₇N₅O₃: 482.22. Found: 481.93.

### Example 141

### N-(pyridin-3-yl)-4-(5-(4-(pyrrolidin-1-yl)benzamido)-1H-benzo[d]imidazol-2-yl)benzamide (Compound 241)

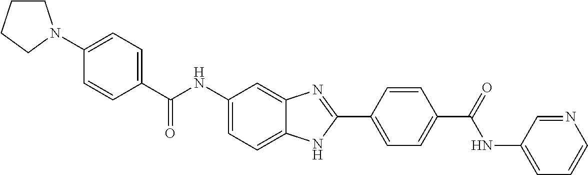 [SMILES: O=C(Nc1cccnc1)c1ccc(-c2nc3cc(NC(=O)c4ccc(N5CCCC5)cc4)ccc3[nH]2)cc1]

Compound 241 was prepared according to the procedure similar to that described in Scheme III from N-(3,4-dinitrophenyl)-4-pyrrolidinylbenzamide and 4-(3-pyridinylaminocarbonyl)benzaldehyde. [M+H]⁺ calcd for C₃₀H₂₆N₆O₂: 503.22. Found: 502.97.

### Example 142

### N-cyclopropyl-4-(5-(4-(4-hydroxypiperidin-1-yl)benzamido)-1H-benzo[d]imidazol-2-yl)benzamide (Compound 242)

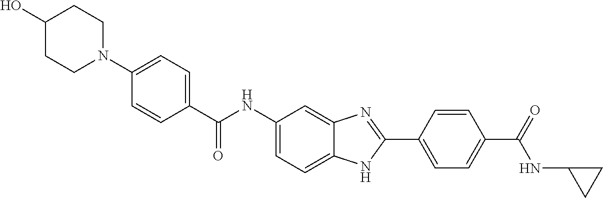 [SMILES: O=C(Nc1ccc2[nH]c(-c3ccc(C(=O)NC4CC4)cc3)nc2c1)c1ccc(N2CCC(O)CC2)cc1]

Compound 242 was prepared according to the procedure similar to that described in Scheme III from N-(3,4-dinitrophenyl)-4-(2-hydroxypiperidinyl)benzamide and 4-cyclopropyl aminocarbonylbenzaldehyde. [M+H]⁺ calcd for C₂₉H₂₉N₅O₃: 496.24. Found: 495.95.

### Example 143

### N-(2-(4-(2-hydroxyethoxy)phenyl)-1H-benzo[d]imidazol-5-yl)-2-(pyrrolidin-1-yl)pyrimidine-5-carboxamide (Compound 243)

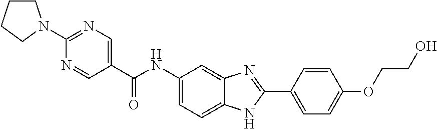 [SMILES: O=C(Nc1ccc2[nH]c(-c3ccc(OCCO)cc3)nc2c1)c1cnc(N2CCCC2)nc1]

Compound 243 was prepared according to the procedure similar to that described in Scheme III from N-(3,4-dinitrophenyl)-2-(pyrrolidin-1-yl)pyrimidine-5-carboxamide and 4-(2-hydroxyethoxy)benzaldehyde. [M+H]⁺ calcd for C₂₄H₂₄N₆O₃: 445.20. Found: 444.87.

### Example 144

### 4-(dimethylamino)-N-(4-(6-(4-(dimethylamino)benzamido)benzo[d]oxazol-2-yl)phenyl)benzamide (Compound 244)

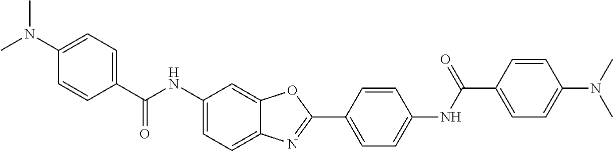

Compound 244 was prepared according to the procedure similar to that described in Scheme III from N-(3-hydroxy-4-nitro)phenyl-4-dimethylaminobenzamide and 4-(4-dimethylaminobenz)amidobenzaldehyde. [M+H]⁺ calcd for C₃₁H₂₉N₅O₃: 520.23. Found: 520.60.

### Example 145

### 4-(dimethylamino)-N-(4-(5-(4-(dimethylamino)benzamido)benzo[d]oxazol-2-yl)phenyl)benzamide (Compound 245)

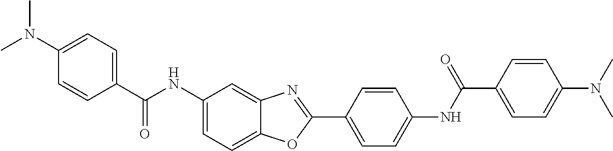

Compound 245 was prepared according to the procedure similar to that described in Scheme III from N-(4-hydroxy-3-nitro)phenyl-4-dimethylaminobenzamide and 4-(4-dimethylaminobenz)amidobenzaldehyde. [M+H]⁺ calcd for C₃₁H₂₉N₅O₃: 520.23. Found: 520.60.

### Example 146

### 4-(dimethylamino)-N-(2-(4-((4-(dimethylamino)phenyl)carbamoyl)phenyl)-1H-indol-5-yl)benzamide (Compound 246)

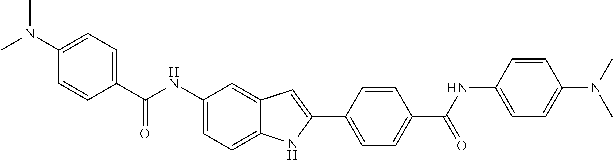

Compound 246 was prepared according to the procedure similar to that described in Scheme III from 5-nitro-2-(4-methoxycarbonylphenyl)indole, 4-dimethylaminoaniline, and 4-dimethylaminobenzoic acid. [M+H]⁺ calcd for C₃₁H₃₀N₆O₂: 519.25. Found: 519.59.

### Example 147

### Methyl 4-(6-(4-(dimethylamino)benzamido)-1H-indol-2-yl)benzoate (Compound 247)

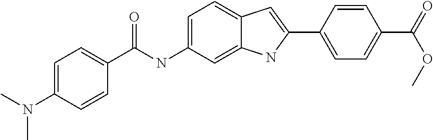 [SMILES: COC(=O)c1ccc(-c2cc3ccc(NC(=O)c4ccc(N(C)C)cc4)cc3[nH]2)cc1]

Compound 247 was prepared according to the procedure similar to that described in Scheme III from 5-nitro-2-(4-methoxycarbonylphenyl)indole and 4-dimethylaminobenzoic acid. [M+H]⁺ calcd for C₂₄H₂₂N₄O₃: 415.17. Found: 415.60.

### Example 148

### 4-((E)-2-(3-methoxybenzylidene)hydrazinecarbonyl)-N-(4-(6-(4-((E)-2-(3-methoxybenzylidene)hydrazinecarbonyl)benzamido)benzo[d]oxazol-2-yl)phenyl)benzamide (Compound 248)

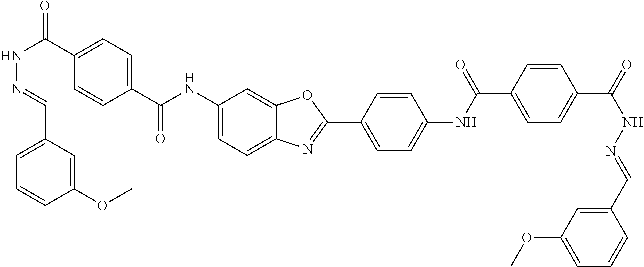 [SMILES: COc1cccc(/C=N/NC(=O)c2ccc(C(=O)Nc3ccc(-c4nc5ccc(NC(=O)c6ccc(C(=O)N/N=C/c7cccc(OC)c7)cc6)cc5o4)cc3)cc2)c1]

Compound 248 was prepared according to the procedure similar to that described in Scheme II from the 2-phenylbenzoxazole-bis-hydrazide and 3-methoxybenzaldehyde. [M+H]⁺ calcd for C₄₅H₃₅N₇O₇: 786.26. Found: 786.23.

### Example 149

### 4-(4-methylpiperazin-1-yl)-N-(2-(4-((4-morpholinophenyl)carbamoyl)phenyl)-1H-benzo[d]imidazol-5-yl)benzamide (Compound 249)

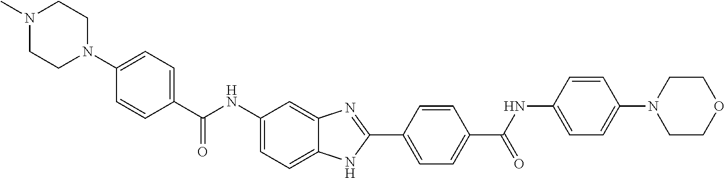 [SMILES: CN1CCN(c2ccc(C(=O)Nc3ccc4[nH]c(-c5ccc(C(=O)Nc6ccc(N7CCOCC7)cc6)cc5)nc4c3)cc2)CC1]

Compound 249 was prepared according to the procedure similar to that described in Scheme III from N-(3,4-dinitrophenyl)-4-(4-methylpiperazinyl)benzamide and 4-(4-morpholinophenyl)aminocarbonylbenzaldehyde. [M+H]⁺ calcd for C₃₆H₃₇N₇O₃: 616.30. Found: 616.09.

### Example 150

### N-(4-(4-methylpiperazin-1-yl)phenyl)-4-(5-(4-morpholinobenzamido)-1H-benzo[d]imidazol-2-yl)benzamide (Compound 250)

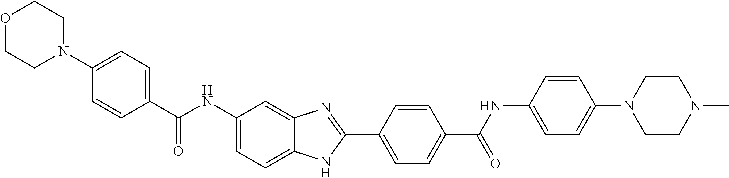 [SMILES: CN1CCN(c2ccc(NC(=O)c3ccc(-c4nc5cc(NC(=O)c6ccc(N7CCOCC7)cc6)ccc5[nH]4)cc3)cc2)CC1]

Compound 250 was prepared according to the procedure similar to that described in Scheme III from N-(3,4-dinitrophenyl)-4-morpholinobenzamide and 4-(4-(4-methylpiperazinyl)phenyl)aminocarbonylbenzaldehyde. [M+H]⁺ calcd for C₃₆H₃₇N₇O₃: 616.30. Found: 616.13.

### Example 151

### 4-(piperazin-1-yl)-N-(2-(4-((4-(piperazin-1-yl)phenyl)carbamoyl)phenyl)-1H-benzo[d]imidazol-5-yl)benzamide (Compound 251)

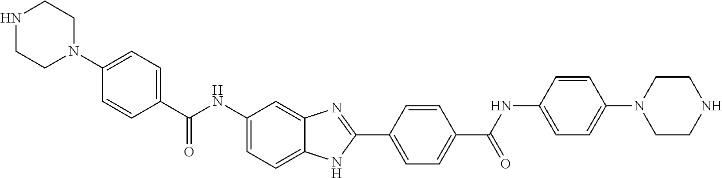 [SMILES: O=C(Nc1ccc(N2CCNCC2)cc1)c1ccc(-c2nc3cc(NC(=O)c4ccc(N5CCNCC5)cc4)ccc3[nH]2)cc1]

Compound 251 was prepared according to the procedure similar to that described in Scheme III from N-(3,4-dinitrophenyl)-4-piperazinylbenzamide and 4-(4-(4-methylpiperazinyl)phenyl)aminocarbonylbenzaldehyde. [M+H]⁺ calcd for C₃₅H₃₆N₈O₂: 601.30. Found: 601.19.

### Example 152

### 4-((2-hydroxyethyl)(methyl)amino)-N-(2-(4-((4-morpholinophenyl)carbamoyl)phenyl)-1H-benzo[d]imidazol-5-yl)benzamide (Compound 252)

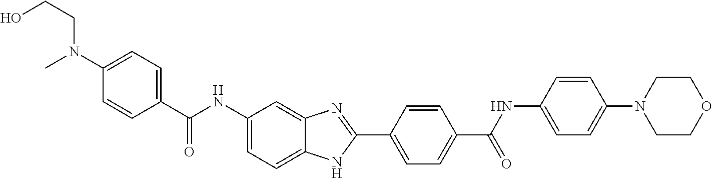 [SMILES: CN(CCO)c1ccc(C(=O)Nc2ccc3[nH]c(-c4ccc(C(=O)Nc5ccc(N6CCOCC6)cc5)cc4)nc3c2)cc1]

Compound 252 was prepared according to the procedure similar to that described in Scheme III from N-(3,4-dinitrophenyl)-4-(N-methyl-N-2-hydroxyethylamino)-benzamide and 4-(4-morpholinophenyl)aminocarbonylbenzaldehyde. [M+H]⁺ calcd for C₃₄H₃₄N₆O₄: 591.26. Found: 591.06.

### Example 153

### 6-morpholino-N-(2-(4-((4-morpholinophenyl)carbamoyl)phenyl)-1H-benzo[d]imidazol-5-yl)nicotinamide (Compound 253)

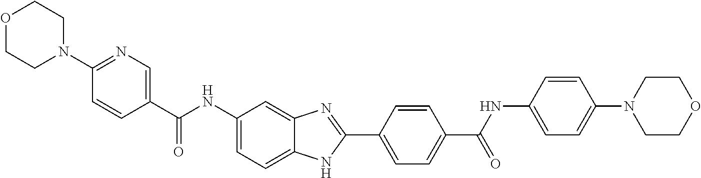 [SMILES: O=C(Nc1ccc(N2CCOCC2)cc1)c1ccc(-c2nc3cc(NC(=O)c4ccc(N5CCOCC5)nc4)ccc3[nH]2)cc1]

Compound 253 was prepared according to the procedure similar to that described in Scheme III from 3,4-dinitro-N-(2-morpholinyl-5-pyridinecarbonyl)aniline and 4-(4-morpholinylphenyl)aminocarbonylbenzaldehyde. [M+H]⁺ calcd for C₃₄H₃₃N₇O₄: 604.26. Found: 604.10.

### Example 154

### 4-morpholino-N-(2-(4-thiomorpholinophenyl)-1H-benzo[d]imidazol-5-yl)benzamide (Compound 254)

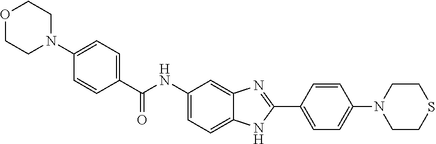 [SMILES: O=C(Nc1ccc2[nH]c(-c3ccc(N4CCSCC4)cc3)nc2c1)c1ccc(N2CCOCC2)cc1]

Compound 254 was prepared according to the procedure similar to that described in Scheme III from 3,4-dinitro-N-(4-thiomorpholinylbenzoyl)aniline and 4-morpholinylbenzaldehyde. [M+H]⁺ calcd for C₂₈H₂₉N₅O₂S: 500.20. Found: 500.00.

### Example 155

### N-(2-(4-(4-hydroxypiperidin-1-yl)phenyl)-1H-benzo[d]imidazol-5-yl)-4-morpholinobenzamide (Compound 255)

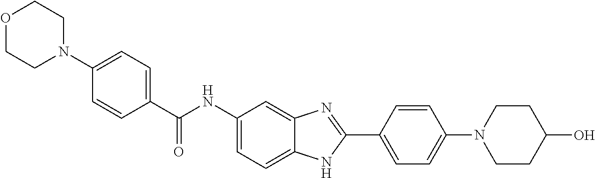 [SMILES: O=C(Nc1ccc2[nH]c(-c3ccc(N4CCC(O)CC4)cc3)nc2c1)c1ccc(N2CCOCC2)cc1]

Compound 255 was prepared according to the procedure similar to that described in Scheme III from 3,4-dinitro-N-(4-morpholinylbenz)aniline and 4-(4-hydroxypiperidinyl)benzaldehyde. [M+H]⁺ calcd for C₂₉H₃₁N₅O₃: 498.24. Found: 497.98.

### Example 156

### 4-morpholino-N-(2-(6-morpholinopyridin-3-yl)-1H-benzo[d]imidazol-5-yl)benzamide (Compound 256)

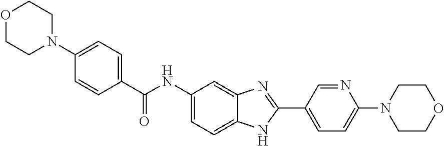 [SMILES: O=C(Nc1ccc2[nH]c(-c3ccc(N4CCOCC4)nc3)nc2c1)c1ccc(N2CCOCC2)cc1]

Compound 256 was prepared according to the procedure similar to that described in Scheme III from 3,4-dinitro-N-(4-morpholinylbenz)aniline and 6-morpholinylpyridine-3-carboxaldehyde. [M+H]⁺ calcd for C₂₇H₂₈N₆O₃: 485.22. Found: 484.99.

### Example 157

### 4-morpholino-N-(2-(4-(morpholinomethyl)phenyl)-1H-benzo[d]imidazol-5-yl)benzamide (Compound 257)

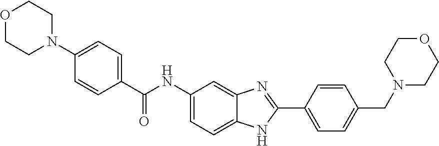 [SMILES: O=C(Nc1ccc2[nH]c(-c3ccc(CN4CCOCC4)cc3)nc2c1)c1ccc(N2CCOCC2)cc1]

Compound 257 was prepared according to the procedure similar to that described in Scheme III from 3,4-dinitro-N-(4-morpholinylbenz)aniline and 4-morpholinomethylbenzaldehyde. [M+H]⁺ calcd for C₂₉H₃₁N₅O₃: 498.24. Found: 498.02.

### Example 158

### N-(2-(4-(3-(dimethylamino)propoxy)phenyl)-1H-benzo[d]imidazol-5-yl)-4-morpholinobenzamide (Compound 258)

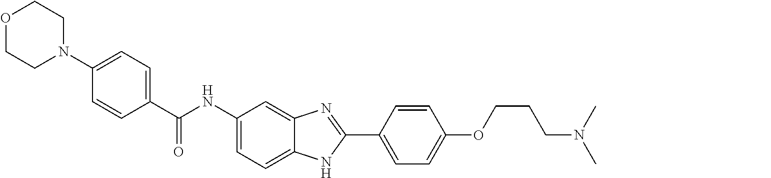 [SMILES: CN(C)CCCOc1ccc(-c2nc3cc(NC(=O)c4ccc(N5CCOCC5)cc4)ccc3[nH]2)cc1]

Compound 258 was prepared according to the procedure similar to that described in Scheme III from 3,4-dinitro-N-(4-morpholinylbenz)aniline and 4-(3-dimethylaminopropyloxy)benzaldehyde. [M+H]⁺ calcd for C₂₉H₃₃N₅O₃: 500.26. Found: 500.01.

### Example 159

### N-(2-(4-morpholinophenyl)-1H-benzo[d]imidazol-5-yl)benzamide (Compound 259)

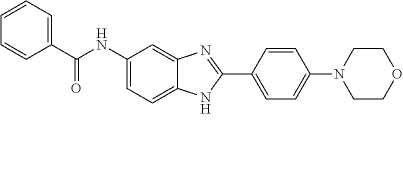 [SMILES: O=C(Nc1ccc2[nH]c(-c3ccc(N4CCOCC4)cc3)nc2c1)c1ccccc1]

Compound 259 was prepared according to the procedure similar to that described in Scheme III from 3,4-dinitro-N-benzoylaniline and 4-morpholinylbenzaldehyde. [M+H]⁺ calcd for C₂₄H₂₂N₄O₂: 399.18. Found: 398.99.

### Example 160

### 2,2′-((1H,3′H-[5,5′-bibenzo[d]imidazole]-2,2′-diylbis(4,1-phenylene))bis(oxy))diethanol (Compound 260)

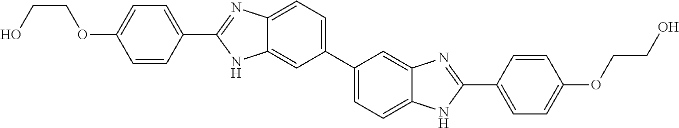 [SMILES: OCCOc1ccc(-c2nc3cc(-c4ccc5nc(-c6ccc(OCCO)cc6)[nH]c5c4)ccc3[nH]2)cc1]

Compound 260 was prepared according to the procedure similar to that described in Scheme III from 3,3′-diaminobenzidine and 4-(2-hydroxyethoxy)benzaldehyde. [M+H]⁺ calcd for C₃₀H₂₆N₄O₄: 507.20. Found: 507.00.

### Example 161

### 4,4′-(1H,3′H-[5,5′-bibenzo[d]imidazole]-2,2′-diyl)bis(N-phenylbenzamide) (Compound 261)

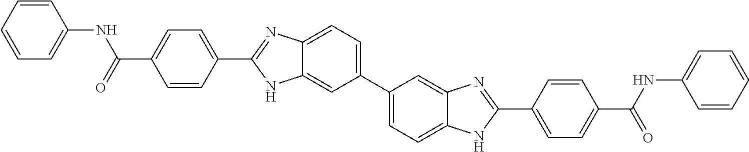 [SMILES: O=C(Nc1ccccc1)c1ccc(-c2nc3cc(-c4ccc5nc(-c6ccc(C(=O)Nc7ccccc7)cc6)[nH]c5c4)ccc3[nH]2)cc1]

Compound 261 was prepared according to the procedure similar to that described in Scheme III from 3,3′-diaminobenzidine and 4-phenylaminocarbonylbenzaldehyde. [M+H]⁺ calcd for C₄₀H₂₈N₆O₂: 625.23. Found: 625.53.

### Example 162

### 2,2′-bis((E)-4-methoxystyryl)-1H,3′H-5,5′-bibenzo[d]imidazole (Compound 262)

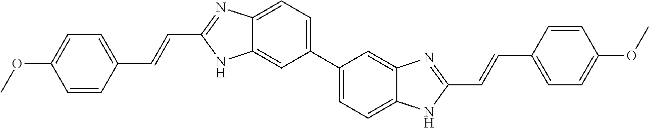

Compound 262 was prepared according to the procedure similar to that described in Scheme III from 3,3′-diaminobenzidine and 3-(4-methoxyphenyl)acrylaldehyde. [M+H]⁺ calcd for C₃₂H₂₆N₄O₂: 499.21. Found: 499.00.

### Example 163

### 4,4′-(propane-1,3-diylbis(oxy))bis(N-(2-(4-methoxyphenyl)-1H-benzo[d]imidazol-5-yl)benzamide) (Compound 263)

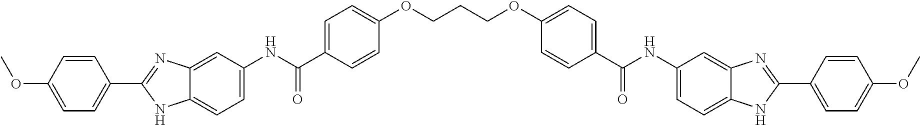

Compound 263 was prepared according to the procedure similar to that described in Scheme III from 1,3-propanedioxybis-(4-benzoic acid) and 5-amino-2-(4-methoxyphenyl)benzimidazole. [M+H]⁺ calcd for C₄₅H₃₈N₆O₆: 759.29. Found: 759.54.

### Example 164

### 4,4′-((1E,1′E)-1H,3′H-[5,5′-bibenzo[d]imidazole]-2,2′-diylbis(ethene-2,1-diyl))bis(N,N-dimethylaniline) (Compound 264)

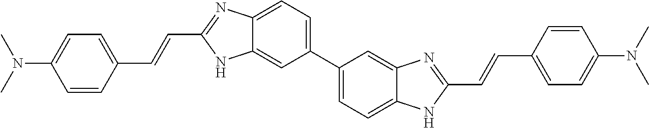

Compound 264 was prepared according to the procedure similar to that described in Scheme III from 3,3′-diaminobenzidine and 3-(4-dimethylaminophenyl)acrylaldehyde. [M+H]⁺ calcd for C₃₄H₃₂N₆: 525.27. Found: 525.59.

### Example 165

### N,N′-(1H,3′H-[5,5′-bibenzo[d]imidazole]-2,2′-diylbis(4,1-phenylene))bis(4-(dimethylamino)benzamide) (Compound 265)

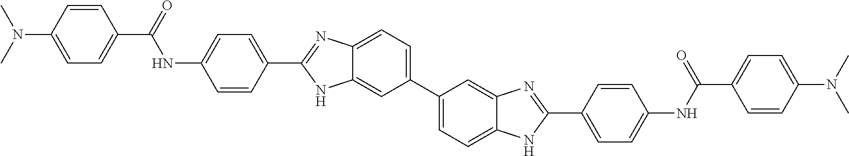

Compound 265 was prepared according to the procedure similar to that described in Scheme III from 3,3′-diaminobenzidine and 4-(4-dimethylaminobenzamido)benzaldehyde. [M+H]⁺ calcd for C₄₄H₃₈N₈O₂: 711.31. Found: 711.38.

### Example 166

### 4,4′-(propane-1,3-diylbis(oxy))bis(N-(2-(trifluoromethyl)-1H-benzo[d]imidazol-5-yl)benzamide) (Compound 266)

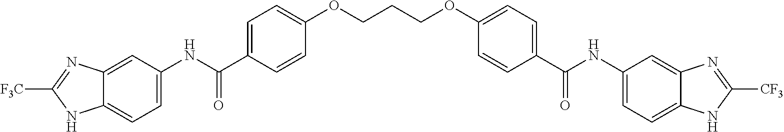 [SMILES: O=C(Nc1ccc2[nH]c(C(F)(F)F)nc2c1)c1ccc(OCCCOc2ccc(C(=O)Nc3ccc4[nH]c(C(F)(F)F)nc4c3)cc2)cc1]

Compound 266 was prepared according to the procedure similar to that described in Scheme III from 1,3-propanedioxybis-(4-benzoic acid) and 5-amino-2-trifluoromethylbenzimidazole. [M+H]⁺ calcd for C₃₃H₂₄F₆N₆O₄: 683.18. Found: 683.04.

### Example 167

### 4,4′-(1H,3′H-[5,5′-bibenzo[d]imidazole]-2,2′-diyl)bis(N-(4-(dimethylamino)phenyl)benzamide) (Compound 267)

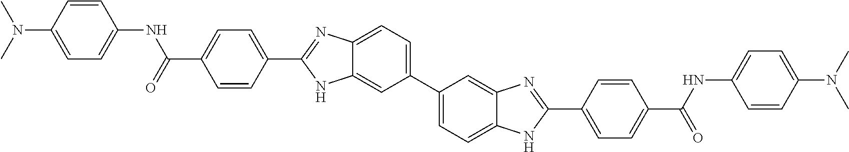

Compound 267 was prepared according to the procedure similar to that described in Scheme III from 3,3′-diaminobenzidine and 4-(4-dimethylaminophenyl)aminocarbonylbenzaldehyde. [M+H]⁺ calcd for C₄₄H₃₈N₈O₂: 711.31. Found: 711.38.

### Example 168

### N,N′-(2,2′-(1,3-phenylene)bis(1H-benzo[d]imidazole-5,2-diyl))bis(4-(pyrrolidin-1-yl)benzamide) (Compound 268)

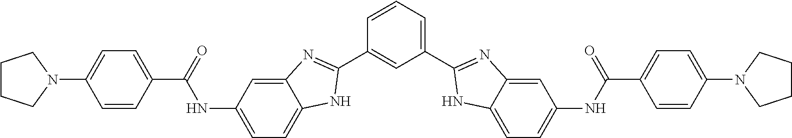 [SMILES: O=C(Nc1ccc2[nH]c(-c3cccc(-c4nc5cc(NC(=O)c6ccc(N7CCCC7)cc6)ccc5[nH]4)c3)nc2c1)c1ccc(N2CCCC2)cc1]

Compound 268 was prepared according to the procedure similar to that described in Scheme III from N-(3,4-dinitrophenyl)-4-pyrrolidinylbenzamide and 1,3-benzbisaldehyde. [M+H]⁺ calcd for C₄₂H₄₈N₈O₂: 687.32. Found: 687.19.

### Example 169

### (E)-4-(2-(3-Methoxybenzylidene)hydrazinecarbonyl)-N-(2-(4-methoxyphenyl)-1H-benzo[d]imidazol-6-yl)benzamide (Compound 269)

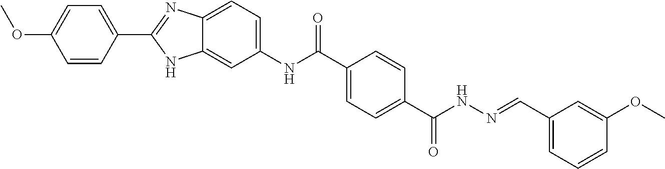

Compound 269 was prepared according to the general procedure described in Scheme V. Preparation of methyl 4-((2-(4-methoxyphenyl)-1H-benzo[d]imidazol-6-yl)carbamoyl)benzoate: 2-(4-Methoxyphenyl)-1H-benzo[d]imidazol-6-amine (120 mg, 0.5 mmol) and methyl 4-(chlorocarbonyl)benzoate (80 mg, 0.4 mmole) were place in a 20 mL vial and pyridine (2.0 mL) added and capped tightly. The reaction mixture was stirred at room temperature for 2 h till a solid precipitated out. To this mixture, 10 mL EtOAC was added and stirred for further 15 minutes then filtered. The filter cake was washed with plenty of water and then with some EtOAc (10 mL) and dried to provide pure title compound (70 mg, 44% yield).

Preparation of 4-(hydrazinecarbonyl)-N-(2-(4-methoxyphenyl)-1H-benzo[d]imidazol-6-yl)benzamide: In a 15-mL sealed tube, methyl 4-((2-(4-methoxyphenyl)-1H-benzo[d]imidazol-6-yl)carbamoyl)benzoate (28 mg, 0.07 mmole) was dissolved in 2 mL absolute ethanol and hydrazine (1 mL) was added. The reaction mixture was heated in an oil bath at 70° C. for overnight. In the morning the reaction mixture was evaporated to dryness to provide the title compound (28 mg, 99%, >95% pure by ¹H-NMR). This compound was used for next step without any further purification.

Preparation of Compound 269: In a 20-mL sealed tube, 4-(hydrazinecarbonyl)-N-(2-(4-methoxyphenyl)-1H-benzo[d]imidazol-6-yl)benzamide (28 mg, 0.07 mmole) was dissolved in 2 mL absolute ethanol then glacial acetic acid (1.0 mL) and sodium acetate (20 mg) was added. The reaction mixture was heated in an oil bath at 72° C. for overnight. In the morning, water (5.0 mL) added and stirred for 15 min. then it was filtered. The filter cake was washed with water, hexanes, ethyl acetate and dried to provide pure compound 269 (10 mg, 27% yield). [M+H]⁺ calcd for C₃₀H₂₅N₅O₄: 520.19. Found: 519.98.

### Example 170

### 2,2′-(1,4-phenylenebis(oxy))bis(N-(2-(trifluoromethyl)-1H-benzo[d]imidazol-5-yl)acetamide) (Compound 270)

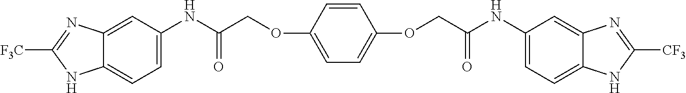 [SMILES: O=C(COc1ccc(OCC(=O)Nc2ccc3[nH]c(C(F)(F)F)nc3c2)cc1)Nc1ccc2[nH]c(C(F)(F)F)nc2c1]

Compound 270 was prepared according to the procedure similar to that described in Scheme V from 1,4-phenyldioxybisacetate and 5-amino-2-trifluoromethylbenzimidazole. [M+H]⁺ calcd for C₂₆H₁₈F6N₆O₄: 593.13. Found: 592.86.

### Example 171

### methyl 4-((2-(trifluoromethyl)-1H-benzo[d]imidazol-5-yl)carbamoyl)benzoate (Compound 271)

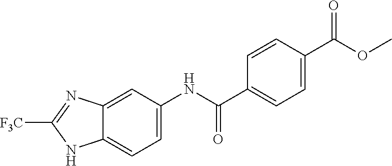 [SMILES: COC(=O)c1ccc(C(=O)Nc2ccc3[nH]c(C(F)(F)F)nc3c2)cc1]

Compound 271 was prepared according to the procedure similar to that described in Scheme V from terephthalic acid mono-ester and 5-amino-2-trifluoromethylbenzimidazole. [M+H]⁺ calcd for C₁₇H₁₂F₃N₃O₃: 364.08. Found: 363.86.

### Example 172

### 2-(4-(dimethylamino)phenyl)-N-(2-(4-methoxyphenyl)-1H-benzo[d]imidazol-5-yl)-1H-benzo[d]imidazole-6-carboxamide (Compound 272)

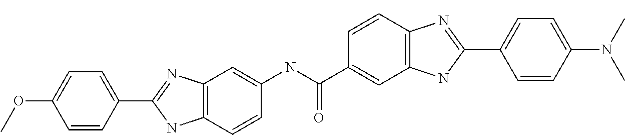

Compound 272 was prepared according to the procedure similar to that described in Scheme V from 2-(4-dimethylaminophenyl)-5-aminobenzimidazole and 2-(4-dimethylaminophenyl)benzimidazole-5-carboxylate. [M+H]⁺ calcd for C₃₀H₂₆N₆O₂: 503.21. Found: 503.25.

### Example 173

### N-(2-(4-(dimethylamino)phenyl)-1H-benzo[d]imidazol-5-yl)-4-(hydrazinecarbonyl)benzamide (Compound 273)

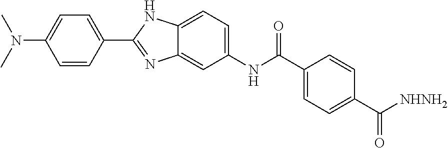 [SMILES: CN(C)c1ccc(-c2nc3cc(NC(=O)c4ccc(C(=O)NN)cc4)ccc3[nH]2)cc1]

Compound 273 was prepared according to the procedure similar to that described in Scheme V from 2-(4-dimethylaminophenyl)-5-aminobenzimidazole and terephthalic acid. [M+H]⁺ calcd for C₂₃H₂₂N₆O₂: 415.18. Found: 414.91.

### Example 174

### N1,N4-bis(2-(4-methoxyphenyl)-1H-benzo[d]imidazol-5-yl)terephthalamide (Compound 274)

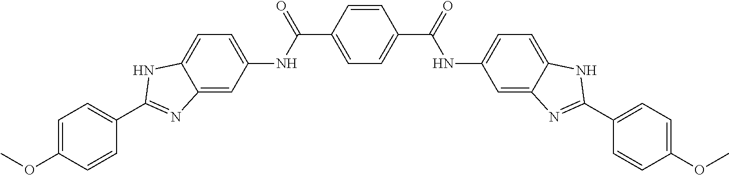

Compound 274 was prepared according to the procedure similar to that described in Scheme V from 2-(4-methoxyphenyl)-5-aminobenzimidazole and terephthalic acid. [M+H]⁺ calcd for C₃₆H₂₈N₆O₄: 609.22. Found: 608.99.

### Example 175

### 2-(4-(dimethylamino)phenyl)-N-(4-(hydrazinecarbonyl)phenyl)-1H-benzo[d]imidazole-5-carboxamide (Compound 275)

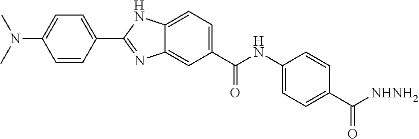 [SMILES: CN(C)c1ccc(-c2nc3cc(C(=O)Nc4ccc(C(=O)NN)cc4)ccc3[nH]2)cc1]

Compound 275 was prepared according to the procedure similar to that described in Scheme V from 2-(4-dimethylaminophenyl)benzimidazole-5-carboxylic acid and 4-aminobenzhydrazide. [M+H]⁺ calcd for C₂₃H₂₂N₆O₂: 415.18. Found: 414.91.

### Example 176

### (E)-4-(2-(3-methoxybenzylidene)hydrazinecarbonyl)-N-(2-(trifluoromethyl)-1H-benzo[d]imidazol-5-yl)benzamide (Compound 276)

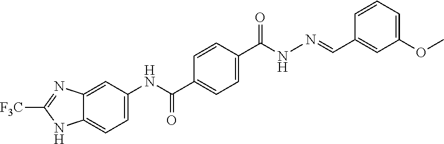 [SMILES: COc1cccc(/C=N/NC(=O)c2ccc(C(=O)Nc3ccc4[nH]c(C(F)(F)F)nc4c3)cc2)c1]

Compound 276 was prepared according to the procedure similar to that described in Scheme V from terephthalic acid mono-ester, 3-methoxybenzaldehyde and 5-amino-2-trifluoromethylbenzimidazole. [M+H]⁺ calcd for C₂₄H₁₈F₃N₅O₃: 482.14. Found: 481.45.

### Example 177

### (E)-4-(2-(4-methoxybenzylidene)hydrazinecarbonyl)-N-(2-(trifluoromethyl)-1H-benzo[d]imidazol-5-yl)benzamide (Compound 277)

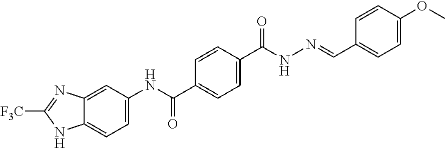 [SMILES: COc1ccc(/C=N/NC(=O)c2ccc(C(=O)Nc3ccc4[nH]c(C(F)(F)F)nc4c3)cc2)cc1]

Compound 277 was prepared according to the procedure similar to that described in Scheme V from terephthalic acid mono-ester, 4-methoxybenzaldehyde and 5-amino-2-trifluoromethylbenzimidazole. [M+H]⁺ calcd for C₂₄H₁₈F₃N₅O₃: 482.14. Found: 481.45.

### Example 178

### (E)-N-(2-(4-(dimethylamino)phenyl)-1H-benzo[d]imidazol-6-yl)-4-(2-(3-methoxybenzylidene)hydrazinecarbonyl)benzamide (Compound 278)

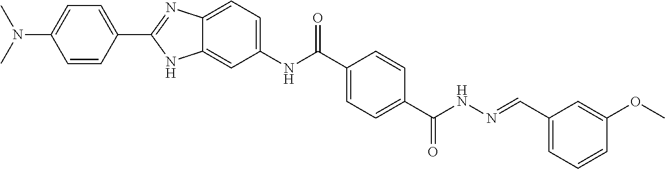

Compound 278 was prepared according to the procedure described in Scheme V from 5-amino-2-(4-dimethylaminophenyl)benzimidazole, terephthalic acid, and 3-methoxybenzaldehyde. [M+H]⁺ calcd for C₃₁H₂₈N₆O₃: 533.22. Found: 532.96.

### Example 179

### (E)-2-(4-(dimethylamino)phenyl)-N-(4-(2-(3-methoxybenzylidene)hydrazinecarbonyl)phenyl)-1H-benzo[d]imidazole-6-carboxamide (Compound 279)

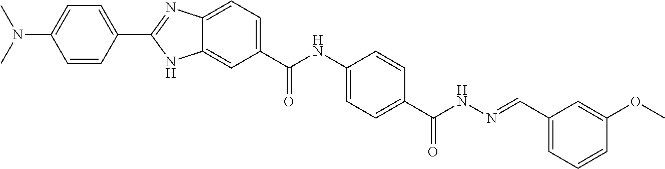

Compound 279 was prepared according to the procedure described in Scheme V from 2-(4-dimethylaminophenyl)benzimidazole-5-carboxylic acid, 4-aminobenzoic acid, and 3-methoxybenzaldehyde. [M+H]⁺ calcd for C₃₁H₂₈N₆O₃: 533.22. Found: 532.95.

### Example 180

### N,N′-(1,4-phenylene)bis(2-(4-morpholinophenyl)-1H-benzo[d]imidazole-5-carboxamide) (Compound 280)

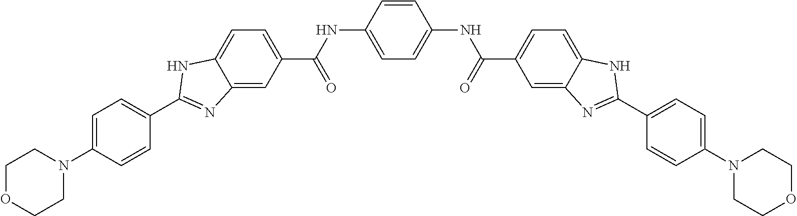 [SMILES: O=C(Nc1ccc(NC(=O)c2ccc3[nH]c(-c4ccc(N5CCOCC5)cc4)nc3c2)cc1)c1ccc2[nH]c(-c3ccc(N4CCOCC4)cc3)nc2c1]

Compound 280 was prepared according to the procedure similar to that described in Scheme V from 2-(4-morpholinylphenyl)benzimidazole-5-carboxylic acid and 1,4-phenylenediamine. [M+H]⁺ calcd for C₄₂H₃₈N₈O₄: 719.30. Found: 719.12.

### Example 181

### N,N′-(1,4-phenylene)bis(2-(4-cyanophenyl)-1H-benzo[d]imidazole-5-carboxamide) (Compound 281)

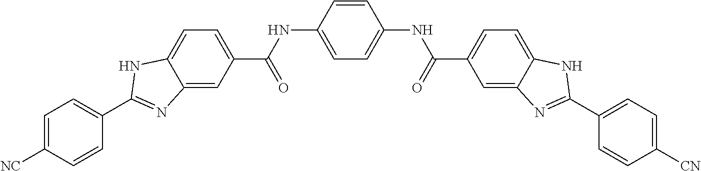 [SMILES: N#Cc1ccc(-c2nc3cc(C(=O)Nc4ccc(NC(=O)c5ccc6[nH]c(-c7ccc(C#N)cc7)nc6c5)cc4)ccc3[nH]2)cc1]

Compound 281 was prepared according to the procedure similar to that described in Scheme V from 2-(4-cyanophenyl)benzimidazole-5-carboxylic acid and 1,4-phenylenediamine. [M+H]⁺ calcd for C₃₆H₂₂N₈O₂: 599.19. Found: 598.97.

### Example 182

### N,N′-(1,4-phenylene)bis(2-(4-(bis(2-hydroxyethyl)amino)phenyl)-1H-benzo[d]imidazole-5-carboxamide) (Compound 282)

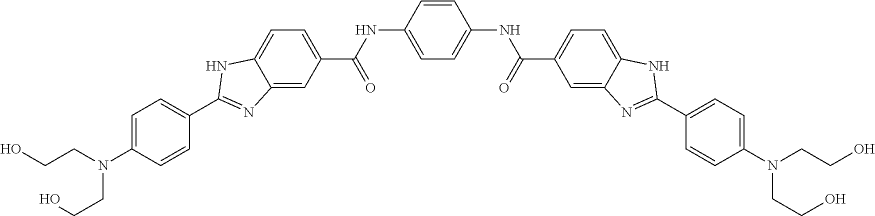 [SMILES: O=C(Nc1ccc(NC(=O)c2ccc3[nH]c(-c4ccc(N(CCO)CCO)cc4)nc3c2)cc1)c1ccc2[nH]c(-c3ccc(N(CCO)CCO)cc3)nc2c1]

Compound 282 was prepared according to the procedure similar to that described in Scheme V from 2-(4-di(2-hydroxyethyl)aminophenyl)benzimidazole-5-carboxylic acid and 1,4-phenylenediamine. [M+H]⁺ calcd for C₄₂H₄₂N₈O₆: 755.32. Found: 755.16.

### Example 183

### N,N′-(1,4-phenylene)bis(2-(4-(dimethylamino)phenyl)-1H-benzo[d]imidazole-5-carboxamide) (Compound 283)

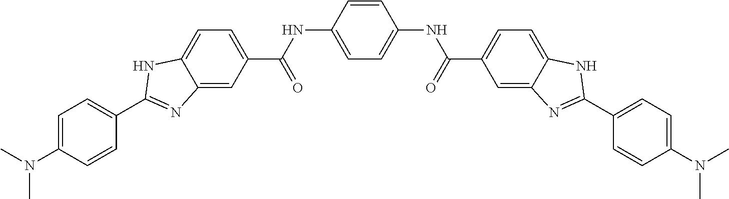

Compound 283 was prepared according to the procedure similar to that described in Scheme V from 2-(4-dimethylaminophenyl)benzimidazole-5-carboxylic acid and 1,4-phenylenediamine. [M+H]⁺ calcd for C₃₈H₃₄N₈O₂: 635.28. Found: 635.05.

### Example 184

### Methyl 4-((2-(4-(2-hydroxyethoxy)phenyl)-1H-benzo[d]imidazol-5-yl)carbamoyl)benzoate (Compound 284)

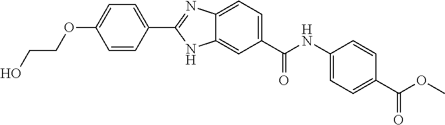 [SMILES: COC(=O)c1ccc(NC(=O)c2ccc3nc(-c4ccc(OCCO)cc4)[nH]c3c2)cc1]

Compound 284 was prepared according to the procedure similar to that described in Scheme V from 2-(4-(2-hydroxyethoxy)phenyl)-5-aminobenzimidazole and terephthalic acid monoester. [M+H]⁺ calcd for C₂₄H₂₁N₃O₅: 432.15. Found: 431.86.

### Example 185

### N⁴,N⁴′-bis(2-chloro-1H-benzo[d]imidazol-5-yl)-[1,1′-biphenyl]-4,4′-dicarboxamide (Compound 285)

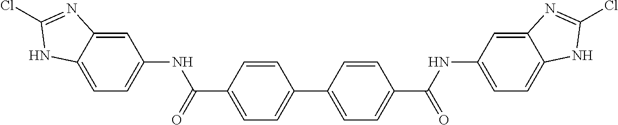 [SMILES: O=C(Nc1ccc2[nH]c(Cl)nc2c1)c1ccc(-c2ccc(C(=O)Nc3ccc4[nH]c(Cl)nc4c3)cc2)cc1]

Compound 285 was prepared according to the procedure similar to that described in Scheme V from 4,4′-bisbenzoic acid and 5-amino-2-chlorobenzimidazole. [M+H]⁺ calcd for C₂₈H₁₈N₆O₂: 541.09. Found: 541.91.

### Example 186

### 4,4′-(1H,3′H-[5,5′-bibenzo[d]imidazole]-2,2′-diylbis(4,1-phenylene))dimorpholine (Compound 286)

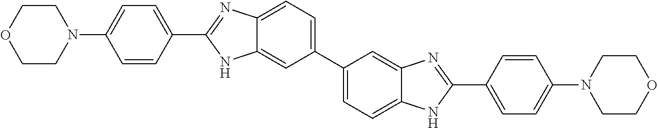 [SMILES: c1cc2[nH]c(-c3ccc(N4CCOCC4)cc3)nc2cc1-c1ccc2nc(-c3ccc(N4CCOCC4)cc3)[nH]c2c1]

Compound 286 was prepared according to the procedure similar to that described in Scheme III from 3,3′-diaminobenzidine and 4-morpholinylbenzaldehyde. [M+H]⁺ calcd for C₃₄H₃₂N₆O₂: 557.26. Found: 557.58.

### Example 187

### (E)-4-(2-(4-(dimethylamino)benzylidene)hydrazinecarbonyl)-N-(2-(4-methoxyphenyl)-1H-benzo[d]imidazol-6-yl)benzamide (Compound 287)

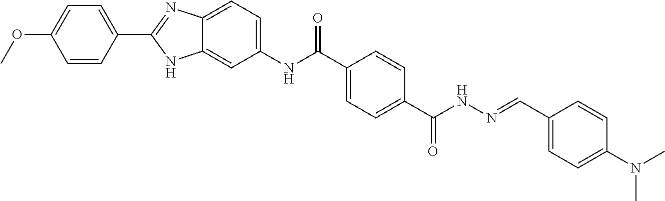

Compound 287 was prepared according to the procedure described in Scheme V from 5-amino-2-(4-methoxyphenyl)benzimidazole, terephthalic acid, and 4-dimethylaminobenzaldehyde. [M+H]⁺ calcd for C₃₁H₂₈N₆O₃: 533.22. Found: 532.55.

### Example 188

### 2-(4-(bis(2-hydroxyethyl)amino)phenyl)-N-(2-(4-morpholinophenyl)-1H-benzo[d]imidazol-5-yl)-1H-benzo[d]imidazole-6-carboxamide (Compound 288)

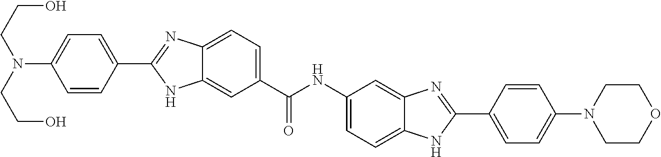 [SMILES: O=C(Nc1ccc2[nH]c(-c3ccc(N4CCOCC4)cc3)nc2c1)c1ccc2nc(-c3ccc(N(CCO)CCO)cc3)[nH]c2c1]

Compound 288 was prepared according to the procedure similar to that described in Scheme V from 2-(4-morpholinophenyl)-5-aminobenzimidazole and 2-(4-N,N-(2-hydroxyethyl)aminophenyl)benzimidazole-5-carboxylate. [M+H]⁺ calcd for C₃₅H₃₅N₇O₄: 618.28. Found: 618.03.

### Example 189

### (2-(4-(bis(2-Hydroxyethyl)amino)phenyl)-N-(2-(4-(bis(2-hydroxyethyl)amino)phenyl)-1H-benzimidazol-5-yl)-1H-benzimidazole-6-carboxamide (Compound 289)

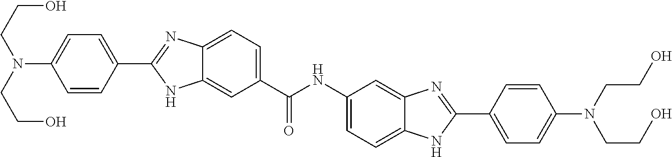 [SMILES: O=C(Nc1ccc2[nH]c(-c3ccc(N(CCO)CCO)cc3)nc2c1)c1ccc2nc(-c3ccc(N(CCO)CCO)cc3)[nH]c2c1]

Compound 289 was prepared according to the procedure similar to that described in Scheme V from 2-(4-(bis(2-hydroxyethyl)amino)phenyl)-5-aminobenzimidazole and 2-(4-(bis(2-hydroxyethyl)amino)phenyl)benzimidazole-5-carboxylate. [M+H]⁺ calcd for C₃₅H₃₇N₇O₅: 636.29. Found: 636.06.

### Example 190

### (2-(4-(bis(2-Hydroxyethyl)amino)phenyl)-N-(2-(4-cyano)phenyl)-1H-benzimidazol-5-yl)-1H-benzimidazole-6-carboxamide (Compound 290)

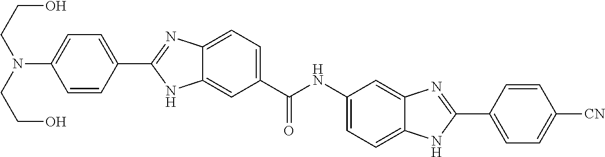 [SMILES: N#Cc1ccc(-c2nc3cc(NC(=O)c4ccc5nc(-c6ccc(N(CCO)CCO)cc6)[nH]c5c4)ccc3[nH]2)cc1]

Compound 290 was prepared according to the procedure similar to that described in Scheme V from 2-(4-cyanophenyl)-5-aminobenzimidazole and 2-(4-(bis(2-hydroxyethyl)amino)phenyl)benzimidazole-5-carboxylate. [M+H]⁺ calcd for C₃₂H₂₇N₇O₃: 558.22. Found: 557.95.

### Example 191

### (2-(4-(bis(2-Hydroxyethyl)amino)phenyl)-N-(2-(4-(2-hydroxy)ethoxy)phenyl)-1H-benzimidazol-5-yl)-1H-benzimidazole-6-carboxamide (Compound 291)

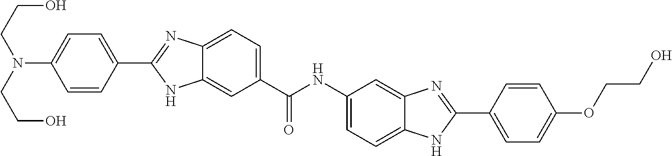 [SMILES: O=C(Nc1ccc2[nH]c(-c3ccc(OCCO)cc3)nc2c1)c1ccc2nc(-c3ccc(N(CCO)CCO)cc3)[nH]c2c1]

Compound 291 was prepared according to the procedure similar to that described in Scheme V from 2-(4-(2-hydroxy)ethoxyphenyl)-5-aminobenzimidazole and 2-(4-(bis(2-hydroxyethyl)amino)phenyl)benzimidazole-5-carboxylate. [M+H]⁺ calcd for C₃₃H₃₂N₆O₅: 593.24. Found: 593.03.

### Example 192

### 2-(4-cyanophenyl)-N-(2-(4-morpholinophenyl)-1H-benzo[d]imidazol-5-yl)-1H-benzo[d]imidazole-6-carboxamide (Compound 292)

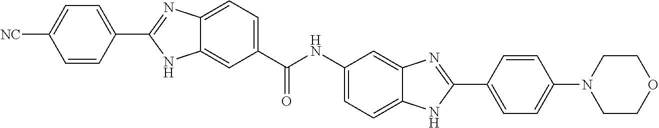 [SMILES: N#Cc1ccc(-c2nc3ccc(C(=O)Nc4ccc5[nH]c(-c6ccc(N7CCOCC7)cc6)nc5c4)cc3[nH]2)cc1]

Compound 292 was prepared according to the procedure similar to that described in Scheme V from 2-(4-morpholinophenyl)-5-aminobenzimidazole and 2-(4-cyanophenyl)benzimidazole-5-carboxylate. [M+H]⁺ calcd for C₃₂H₂₅N₇O₂: 540.21. Found: 539.97.

### Example 193

### N-(2-(4-(bis(2-hydroxyethyl)amino)phenyl)-1H-benzo[d]imidazol-5-yl)-2-(4-cyanophenyl)-1H-benzo[d]imidazole-6-carboxamide (Compound 293)

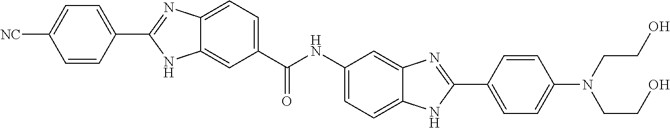 [SMILES: N#Cc1ccc(-c2nc3ccc(C(=O)Nc4ccc5[nH]c(-c6ccc(N(CCO)CCO)cc6)nc5c4)cc3[nH]2)cc1]

Compound 293 was prepared according to the procedure similar to that described in Scheme V from 2-(4-(bis(2-hydroxyethyl)amino)phenyl)-5-aminobenzimidazole and 2-(4-cyanophenyl)benzimidazole-5-carboxylate. [M+H]⁺ calcd for C₃₂H₂₅N₇O₃: 558.22. Found: 557.99.

### Example 194

### 2-(4-cyanophenyl)-N-(2-(4-(2-hydroxyethoxy)phenyl)-1H-benzo[d]imidazol-5-yl)-1H-benzo[d]imidazole-6-carboxamide (Compound 294)

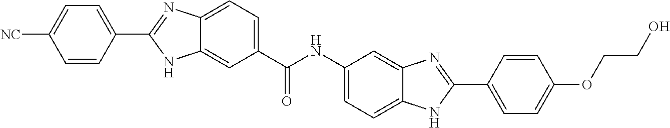 [SMILES: N#Cc1ccc(-c2nc3ccc(C(=O)Nc4ccc5[nH]c(-c6ccc(OCCO)cc6)nc5c4)cc3[nH]2)cc1]

Compound 294 was prepared according to the procedure similar to that described in Scheme V from 2-(4-(2-hydroxyethoxy)amino)phenyl)-5-aminobenzimidazole and 2-(4-cyanophenyl)benzimidazole-5-carboxylate. [M+H]⁺ calcd for C₃₀H₂₂N₆O₃: 515.18. Found: 514.92.

### Example 195

### 2-(4-cyanophenyl)-N-(2-(4-cyanophenyl)-1H-benzo[d]imidazol-5-yl)-1H-benzo[d]imidazole-6-carboxamide (Compound 295)

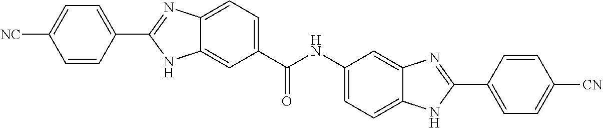 [SMILES: N#Cc1ccc(-c2nc3cc(NC(=O)c4ccc5nc(-c6ccc(C#N)cc6)[nH]c5c4)ccc3[nH]2)cc1]

Compound 295 was prepared according to the procedure similar to that described in Scheme V from 2-(4-cyano)phenyl)-5-aminobenzimidazole and 2-(4-cyanophenyl)benzimidazole-5-carboxylate. [M+H]⁺ calcd for C₂₉H₁₇N₇O: 480.15. Found: 479.89.

### Example 196

### N-(2-(4-(bis(2-hydroxyethyl)amino)phenyl)-1H-benzo[d]imidazol-5-yl)-2-(4-morpholinophenyl)-1H-benzo[d]imidazole-6-carboxamide (Compound 296)

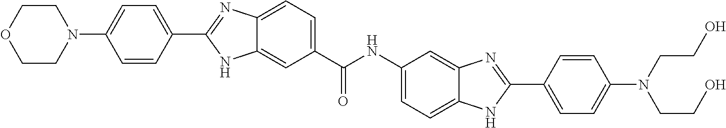 [SMILES: O=C(Nc1ccc2[nH]c(-c3ccc(N(CCO)CCO)cc3)nc2c1)c1ccc2nc(-c3ccc(N4CCOCC4)cc3)[nH]c2c1]

Compound 296 was prepared according to the procedure similar to that described in Scheme V from 2-(4-(bis(2-hydroxyethyl)amino)phenyl)-5-aminobenzimidazole and 2-(4-morpholinophenyl)benzimidazole-5-carboxylate. [M+H]⁺ calcd for C₃₅H₃₅N₇O₄: 618.28. Found: 618.03.

### Example 197

### N-(2-(4-(2-hydroxyethoxy)phenyl)-1H-benzo[d]imidazol-5-yl)-2-(4-morpholinophenyl)-1H-benzo[d]imidazole-6-carboxamide (Compound 297)

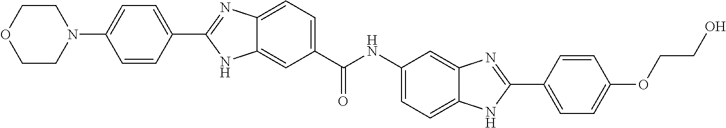 [SMILES: O=C(Nc1ccc2[nH]c(-c3ccc(OCCO)cc3)nc2c1)c1ccc2nc(-c3ccc(N4CCOCC4)cc3)[nH]c2c1]

Compound 297 was prepared according to the procedure similar to that described in Scheme V from 2-(4-(2-hydroxyethoxy)amino)phenyl)-5-aminobenzimidazole and 2-(4-morpholinophenyl)benzimidazole-5-carboxylate. [M+H]calcd for C₃₃H₃₀N₆O₄: 575.23. Found: 575.00.

### Example 198

### N-(2-(4-cyanophenyl)-1H-benzo[d]imidazol-5-yl)-2-(4-morpholinophenyl)-1H-benzo[d]imidazole-6-carboxamide (Compound 298)

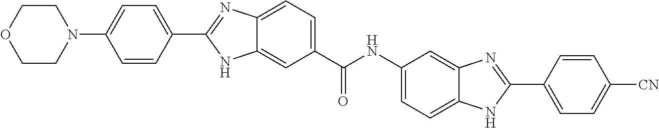 [SMILES: N#Cc1ccc(-c2nc3cc(NC(=O)c4ccc5nc(-c6ccc(N7CCOCC7)cc6)[nH]c5c4)ccc3[nH]2)cc1]

Compound 298 was prepared according to the procedure similar to that described in Scheme V from 2-(4-cyano)phenyl)-5-aminobenzimidazole and 2-(4-morpholinophenyl)benzimidazole-5-carboxylate. [M+H]⁺ calcd for C₃₂H₂₅N₇O₂: 540.21. Found: 539.97.

### Example 199

### 2-(4-(2-hydroxyethoxy)phenyl)-N-(2-(4-morpholinophenyl)-1H-benzo[d]imidazol-5-yl)-1H-benzo[d]imidazole-6-carboxamide (Compound 299)

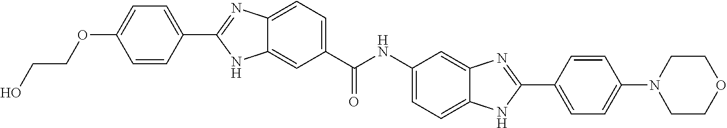 [SMILES: O=C(Nc1ccc2[nH]c(-c3ccc(N4CCOCC4)cc3)nc2c1)c1ccc2nc(-c3ccc(OCCO)cc3)[nH]c2c1]

Compound 299 was prepared according to the procedure similar to that described in Scheme V from 2-(4-morpholinophenyl)-5-aminobenzimidazole and 2-(4-(2-hydroxyethoxy)phenyl)benzimidazole-5-carboxylate. [M+H]⁺ calcd for C₃₃H₃₀N₆O₄: 575.23. Found: 575.07.

### Example 200

### N,N′-(oxybis(4,1-phenylene))bis(4-(dimethylamino)benzamide) (Compound 300)

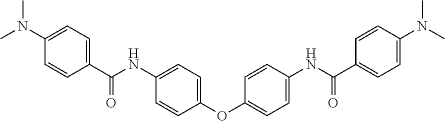

Compound 300 was prepared according to the procedure described in Scheme IV from 4,4′-oxybisphenylamine and 4-dimethylaminobenzoate. [M+H]⁺ calcd for C₃₀H₃₁N₄O₃: 495.24. Found: 495.01

### Example 201

### N,N′-(carbonylbis(4,1-phenylene))bis(4-(dimethylamino)benzamide) (Compound 301)

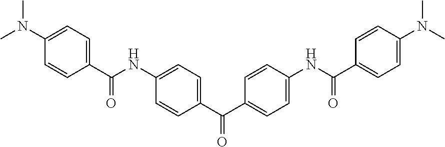

Compound 301 was prepared according to the procedure described in Scheme IV. 4-(Dimethylamino)benzoyl chloride (Aldrich, 151 mg, 0.825 mmol) in 1 mL of methylene chloride was slowly added over 5 min to 4,4′-diaminobenzophenone (Aldrich, 70 mg, 0.330 mmol) in 3 mL of methylene chloride containing 0.3 mL of pyridine. The reaction was allowed to stir at room temperature for 12 h and filtered. The white precipitate was washed with water (5 mL), ethanol (2 mL) and dried under vacuum to yield 165 mg of N,N′-(carbonylbis(4,1-phenylene))bis(4-(dimethylamino)benzamide) as a white powder. ¹H NMR (500 MHz, DMSO-d₆) δ 10.19 (s, 2H), 7.97 (d, J=8.7 Hz, 4H), 7.89 (d, J=8.5 Hz, 4H), 7.77 (d, J=8.5 Hz, 4H), 6.77 (d, J=8.7 Hz, 4H), 3.00 (s, 12H).

### Example 202

### N,N′-(carbonylbis(4,1-phenylene))bis(4-morpholinobenzamide) (Compound 302)

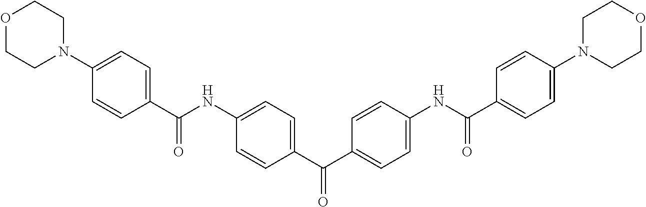 [SMILES: O=C(Nc1ccc(C(=O)c2ccc(NC(=O)c3ccc(N4CCOCC4)cc3)cc2)cc1)c1ccc(N2CCOCC2)cc1]

Compound 302 was prepared according to the procedure described in Scheme IV from 4,4′-diaminobenzophenone and 4-morpholinylbenzoate. [M+H]⁺ calcd for C₃₅H₃₅N₄O₅: 591.26. Found: 591.20

### Example 203

### N,N′-(((methoxyimino)methylene)bis(4,1-phenylene))bis(4-morpholinobenzamide) (Compound 303)

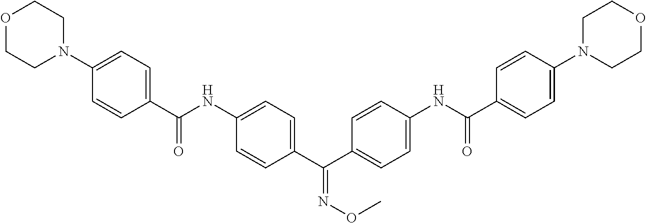 [SMILES: CON=C(c1ccc(NC(=O)c2ccc(N3CCOCC3)cc2)cc1)c1ccc(NC(=O)c2ccc(N3CCOCC3)cc2)cc1]

Compound 303 was prepared from compound 302 by a standard oxime synthesis procedure. [M+H]⁺ calcd for C₃₆H₃₇N₅O₅: 620.24. Found: 620.11.

### Example 204

### N,N′-(((hydroxyimino)methylene)bis(4,1-phenylene))bis(4-morpholinobenzamide) (Compound 304)

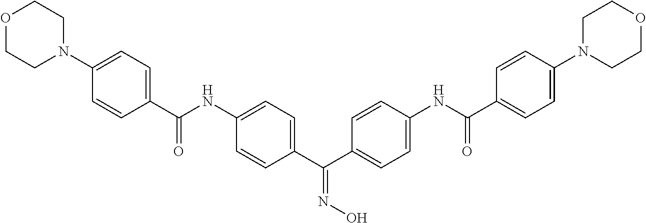 [SMILES: O=C(Nc1ccc(C(=NO)c2ccc(NC(=O)c3ccc(N4CCOCC4)cc3)cc2)cc1)c1ccc(N2CCOCC2)cc1]

Compound 304 was prepared from compound 302 by a standard oxime synthesis procedure. [M+H]⁺ calcd for C₃₅H₃₅N₅O₅: 606.27. Found: 606.06.

### Example 205

### N,N′-((hydroxymethylene)bis(4,1-phenylene))bis(4-(dimethylamino)benzamide) (Compound 305)

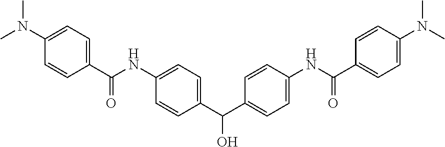

Compound 305 was prepared from compound 301 by a standard reduction condition. [M+H]⁺ calcd for C₃₁H₃₂N₄O₃: 509.14. Found: 508.98.

### Example 206

### N,N′-(((hydroxyimino)methylene)bis(4,1-phenylene))bis(4-(dimethylamino)benzamide) (Compound 306)

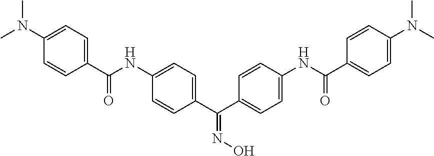

Compound 306 was prepared from compound 301 by a standard oxime synthesis procedure. [M+H]⁺ calcd for C₃₁H₃₁N₅O₃: 522.25. Found: 522.01.

### Example 207

### N,N′-(((ethoxyimino)methylene)bis(4,1-phenylene))bis(4-(dimethylamino)benzamide) (Compound 307)

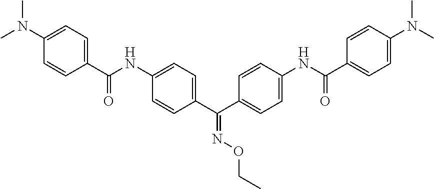

Compound 307 was prepared from compound 301 by a standard oxime synthesis procedure. [M+H]⁺ calcd for C₃₃H₃₅N₅O₃: 550.28. Found: 550.02.

### Example 208

### N,N′-((((benzyloxy)imino)methylene)bis(4,1-phenylene))bis(4-(dimethylamino)benzamide) (Compound 308)

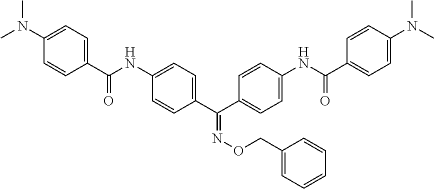

Compound 308 was prepared from compound 301 by a standard oxime synthesis procedure. [M+H]⁺ calcd for C₃₈H₃₇N₅O₃: 612.29. Found: 612.07.

### Example 209

### N,N′-((((allyloxy)imino)methylene)bis(4,1-phenylene))bis(4-(dimethylamino)benzamide) (Compound 309)

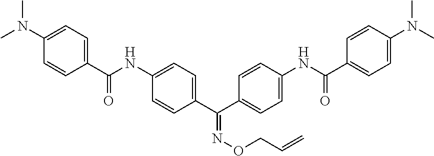

Compound 309 was prepared from compound 301 by a standard oxime synthesis procedure. [M+H]⁺ calcd for C₃₄H₃₅N₅O₃: 562.28. Found: 562.04.

### Example 210

### N,N′-((((2-phenylacetoxy)imino)methylene)bis(4,1-phenylene))bis(4-(dimethylamino)benzamide) (Compound 310)

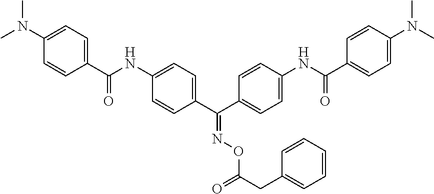

Compound 310 was prepared from compound 301 by a standard oxime synthesis procedure. [M+H]⁺ calcd for C₃₉H₃₇N₅O₄: 640.29. Found: 640.23.

### Example 211

### N,N′-(((acetoxyimino)methylene)bis(4,1-phenylene))bis(4-(dimethylamino)benzamide) (Compound 311)

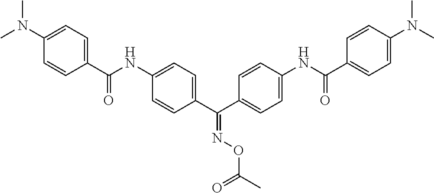

Compound 311 was prepared from compound 301 by a standard oxime synthesis procedure. [M+H]⁺ calcd for C₃₃H₃₃N₅O₄: 564.26. Found: 564.00.

### Example 212

### N,N′-(((((thiophene-2-carbonyl)oxy)imino)methylene)bis(4,1-phenylene))bis(4-(dimethylamino)benzamide) (Compound 312)

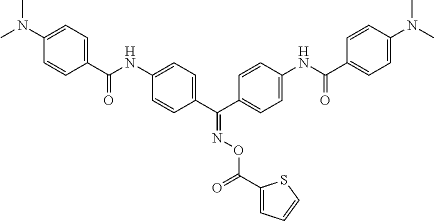

Compound 312 was prepared from compound 301 by a standard oxime synthesis procedure. [M+H]⁺ calcd for C₃₆H₃₃N₅O₄S: 632.77. Found: 631.99.

### Example 213

### N,N′-((((benzoyloxy)imino)methylene)bis(4,1-phenylene))bis(4-(dimethylamino)benzamide) (Compound 313)

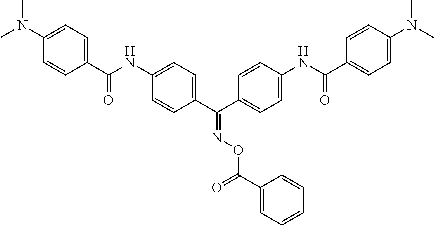

Compound 313 was prepared from compound 301 by a standard oxime synthesis procedure. [M+Na]⁺ calcd for C₃₈H₃₅N₅O₄: 647.74. Found: 647.86.

### Example 214

### N,N′-(carbonylbis(4,1-phenylene))bis(4-(3-hydroxypyrrolidin-1-yl)benzamide) (Compound 314)

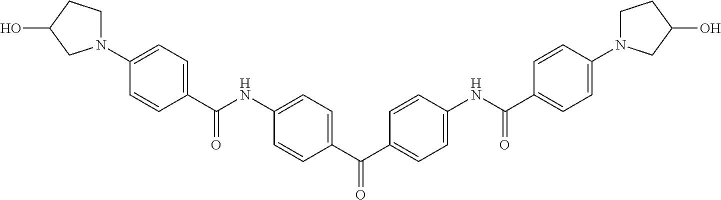 [SMILES: O=C(Nc1ccc(C(=O)c2ccc(NC(=O)c3ccc(N4CCC(O)C4)cc3)cc2)cc1)c1ccc(N2CCC(O)C2)cc1]

Compound 314 was prepared according to the procedure described in Scheme IV from 4,4′-diaminobenzophenone and 4-(2-hydroxypyrrolidino)benzoate. [M+H]⁺ calcd for C₃₅H₃₅N₄O₅: 591.26. Found: 591.03.

### Example 215

### N,N′-(((methoxyimino)methylene)bis(4,1-phenylene))bis(4-(3-hydroxypyrrolidin-1-yl)benzamide) (Compound 315)

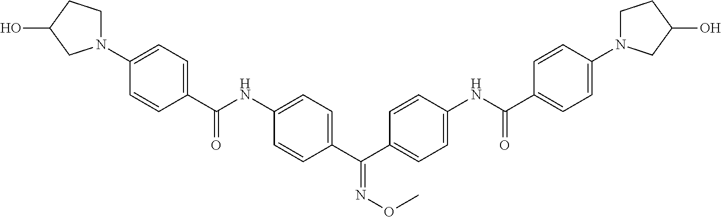 [SMILES: CON=C(c1ccc(NC(=O)c2ccc(N3CCC(O)C3)cc2)cc1)c1ccc(NC(=O)c2ccc(N3CCC(O)C3)cc2)cc1]

Compound 315 was prepared from compound 314 by a standard oxime synthesis procedure. [M+H]⁺ calcd for C₃₆H₃₇N₅O₅: 620.28. Found: 620.11.

### Example 216

### N,N′-(carbonylbis(4,1-phenylene))bis(4-(4-methylpiperazin-1-yl)benzamide) (Compound 316)

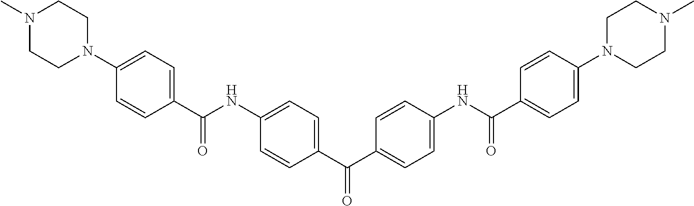

Compound 316 was prepared according to the procedure described in Scheme IV from 4,4′-diaminobenzophenone and 4-(4-methylpiperazino)benzoate. [M+H]⁺ calcd for C₃₇H₄₁N₆O₃: 617.32. Found: 617.12.

### Example 217

### N,N′-(carbonylbis(4,1-phenylene))bis(4-(piperazin-1-yl)benzamide) (Compound 317)

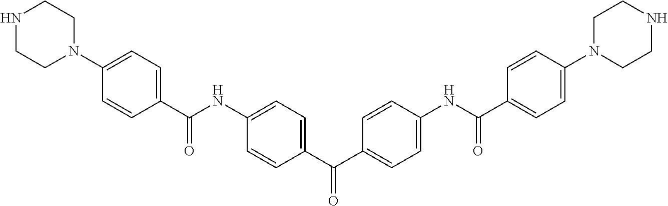 [SMILES: O=C(Nc1ccc(C(=O)c2ccc(NC(=O)c3ccc(N4CCNCC4)cc3)cc2)cc1)c1ccc(N2CCNCC2)cc1]

Compound 317 was prepared according to the procedure described in Scheme IV from 4,4′-diaminobenzophenone and 4-piperazinobenzoate. [M+H]⁺ calcd for C₃₅H₃₇N₆O₃: 589.29. Found: 589.07.

### Example 218

### N,N′-(carbonylbis(4,1-phenylene))bis(4-(4-(3-hydroxypropyl)piperazin-1-yl)benzamide) (Compound 318)

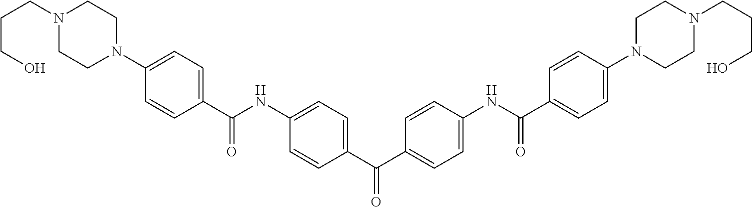 [SMILES: O=C(Nc1ccc(C(=O)c2ccc(NC(=O)c3ccc(N4CCN(CCCO)CC4)cc3)cc2)cc1)c1ccc(N2CCN(CCCO)CC2)cc1]

Compound 318 was prepared according to the procedure described in Scheme IV from 4,4′-diaminobenzophenone and 4-(4-(3-hydroxypropyl)piperazino)benzoate. [M+H]⁺ calcd for C₄₁H₄₈N₆O₅: 705.37. Found: 705.19.

### Example 219

### N,N′-(carbonylbis(4,1-phenylene))bis(4-(3,5-dimethylpiperazin-1-yl)benzamide) (Compound 319)

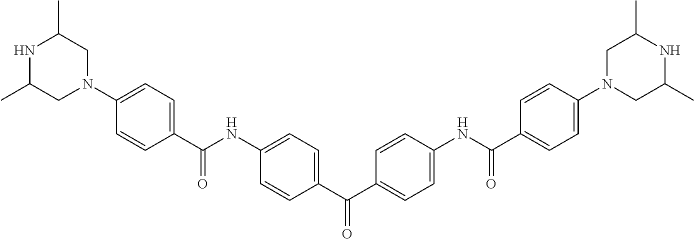

Compound 319 was prepared according to the procedure described in Scheme IV from 4,4′-diaminobenzophenone and 4-(3,5-dimethylpiperazino)benzoate. [M+H]⁺ calcd for C₃₉H₄₄N₆O₃: 645.35. Found: 645.16.

### Example 220

### N,N′-(carbonylbis(4,1-phenylene))bis(4-(4-(2-(2-hydroxyethoxy)ethyl)piperazin-1-yl)benzamide) (Compound 320)

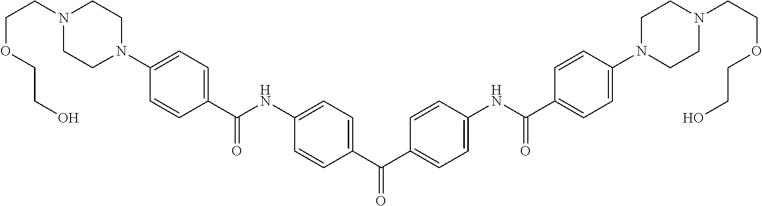 [SMILES: O=C(Nc1ccc(C(=O)c2ccc(NC(=O)c3ccc(N4CCN(CCOCCO)CC4)cc3)cc2)cc1)c1ccc(N2CCN(CCOCCO)CC2)cc1]

Compound 320 was prepared according to the procedure described in Scheme IV from 4,4′-diaminobenzophenone and 4-(4-(2-hydroxyethoxy)ethyl)piperazino)benzoate. [M+H]⁺ calcd for C₄₃H₅₂N₆O₇: 765.39. Found: 765.36.

### Example 221

### N,N′-(carbonylbis(4,1-phenylene))bis(4-(4-(cyclopropanecarbonyl)piperazin-1-yl)benzamide) (Compound 321)

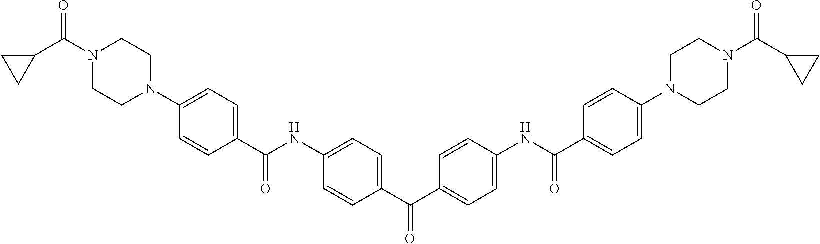 [SMILES: O=C(Nc1ccc(C(=O)c2ccc(NC(=O)c3ccc(N4CCN(C(=O)C5CC5)CC4)cc3)cc2)cc1)c1ccc(N2CCN(C(=O)C3CC3)CC2)cc1]

Compound 321 was prepared according to the procedure described in Scheme IV from 4,4′-diaminobenzophenone and 4-(4-cyclopropylcarbonyl)piperazino)benzoate. [M+H]⁺ calcd for C₄₃H₄₄N₆O₅: 725.34. Found: 725.11.

### Example 222

### N,N′-(carbonylbis(4,1-phenylene))bis(4-(piperidin-1-yl)benzamide) (Compound 322)

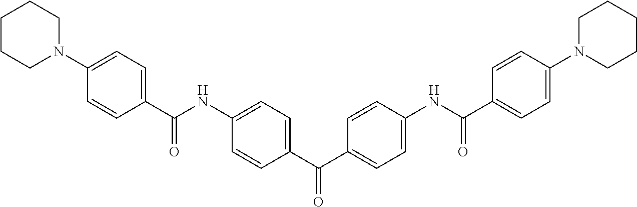 [SMILES: O=C(Nc1ccc(C(=O)c2ccc(NC(=O)c3ccc(N4CCCCC4)cc3)cc2)cc1)c1ccc(N2CCCCC2)cc1]

Compound 322 was prepared according to the procedure described in Scheme IV from 4,4′-diaminobenzophenone and 4-piperidinobenzoate. [M+H]⁺ calcd for C₃₇H₃₈N₄O₃: 587.30. Found: 587.02.

### Example 223

### N,N′-(carbonylbis(4,1-phenylene))bis(4-(4-hydroxypiperidin-1-yl)benzamide) (Compound 323)

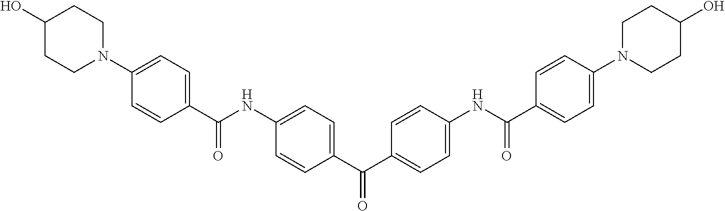 [SMILES: O=C(Nc1ccc(C(=O)c2ccc(NC(=O)c3ccc(N4CCC(O)CC4)cc3)cc2)cc1)c1ccc(N2CCC(O)CC2)cc1]

Compound 323 was prepared according to the procedure described in Scheme IV from 4,4′-diaminobenzophenone and 4-(4-hydroxypiperidino)benzoate. [M+H]⁺ calcd for C₃₇H₃₉N₄O₅: 619.29. Found: 619.10.

### Example 224

### N,N′-(carbonylbis(4,1-phenylene))bis(4-(pyrrolidin-1-yl)benzamide) (Compound 324)

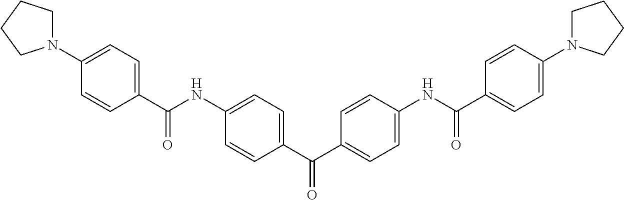 [SMILES: O=C(Nc1ccc(C(=O)c2ccc(NC(=O)c3ccc(N4CCCC4)cc3)cc2)cc1)c1ccc(N2CCCC2)cc1]

Compound 324 was prepared according to the procedure described in Scheme IV from 4,4′-diaminobenzophenone and 4-pyrrolidinobenzoate. [M+H]⁺ calcd for C₃₅H₃₄N₄O₃: 559.27. Found: 559.00.

### Example 225

### N,N′-(carbonylbis(4,1-phenylene))bis(4-(1H-pyrrol-1-yl)benzamide) (Compound 325)

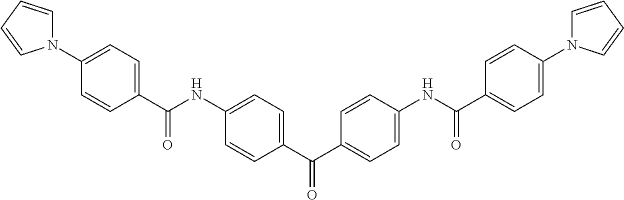 [SMILES: O=C(Nc1ccc(C(=O)c2ccc(NC(=O)c3ccc(-n4cccc4)cc3)cc2)cc1)c1ccc(-n2cccc2)cc1]

Compound 325 was prepared according to the procedure described in Scheme IV from 4,4′-diaminobenzophenone and 4-pyrrolylbenzoate. [M+H]⁺ calcd for C₃₅H₂₆N₄O₃: 551.20. Found: 551.04.

### Example 226

### N,N′-(carbonylbis(4,1-phenylene))bis(4-(1H-imidazol-1-yl)benzamide) (Compound 326)

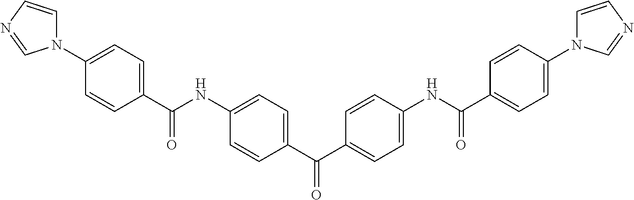 [SMILES: O=C(Nc1ccc(C(=O)c2ccc(NC(=O)c3ccc(-n4ccnc4)cc3)cc2)cc1)c1ccc(-n2ccnc2)cc1]

Compound 326 was prepared according to the procedure described in Scheme IV from 4,4′-diaminobenzophenone and 4-imidazolylbenzoate. [M+H]⁺ calcd for C₃₃H₂₄N₆O₃: 553.19. Found: 552.98.

### Example 227

### N,N′-(carbonylbis(4,1-phenylene))bis(4-(bis(2-hydroxyethyl)amino)benzamide)

(Compound 327)

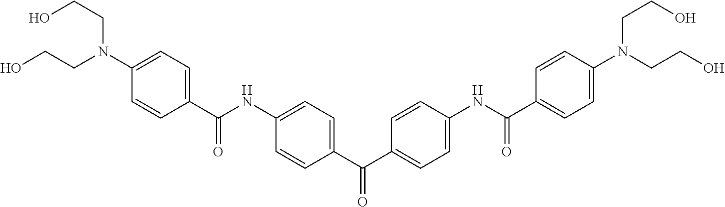 [SMILES: O=C(Nc1ccc(C(=O)c2ccc(NC(=O)c3ccc(N(CCO)CCO)cc3)cc2)cc1)c1ccc(N(CCO)CCO)cc1]

Compound 327 was prepared according to the procedure described in Scheme IV from 4,4′-diaminobenzophenone and 4-bis(2-hydroxylethyl)aminobenzoate. [M+H]⁺ calcd for C₃₅H₃₈N₄O₇: 627.28. Found: 627.06.

### Example 228

### N,N′-(carbonylbis(4,1-phenylene))bis(1-methyl-1H-indole-5-carboxamide) (Compound 328)

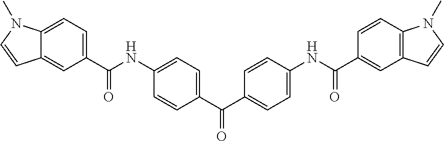 [SMILES: Cn1ccc2cc(C(=O)Nc3ccc(C(=O)c4ccc(NC(=O)c5ccc6c(ccn6C)c5)cc4)cc3)ccc21]

Compound 328 was prepared according to the procedure described in Scheme IV from 4,4′-diaminobenzophenone and 5-(1-methylindole)carboxylate. [M+H]⁺ calcd for C₃₃H₂₆N₄O₃: 527.20. Found: 527.00.

### Example 229

### N,N′-(carbonylbis(4,1-phenylene))bis(4-methyl-3,4-dihydro-2H-benzo[b][1,4]oxazine-7-carboxamide) (Compound 329)

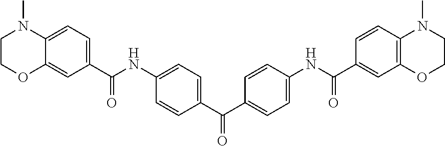 [SMILES: CN1CCOc2cc(C(=O)Nc3ccc(C(=O)c4ccc(NC(=O)c5ccc6c(c5)OCCN6C)cc4)cc3)ccc21]

Compound 329 was prepared according to the procedure described in Scheme IV from 4,4′-diaminobenzophenone and 6-(1-methyl-1,4-benzoxazine)carboxylate. [M+H]⁺ calcd for C₃₃H₃₀N₄O₅: 563.22. Found: 562.92.

### Example 230

### N,N′-(carbonylbis(4,1-phenylene))bis(4-((2-hydroxyethyl)(methyl)amino)benzamide) (Compound 330)

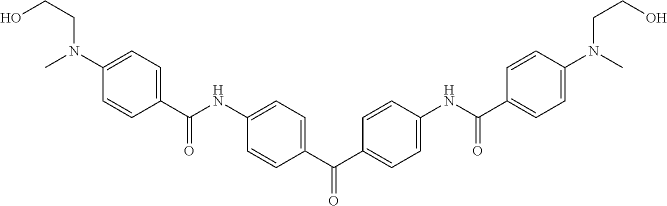 [SMILES: CN(CCO)c1ccc(C(=O)Nc2ccc(C(=O)c3ccc(NC(=O)c4ccc(N(C)CCO)cc4)cc3)cc2)cc1]

Compound 330 was prepared according to the procedure described in Scheme IV from 4,4′-diaminobenzophenone and 4-(N-2-hydroxylethyl-N-methylamino)benzoate. [M+H]⁺ calcd for C₃₃H₃₅N₄O₅: 567.26. Found: 567.04.

### Example 231

### N,N′-(((2-phenylhydrazono)methylene)bis(4,1-phenylene))bis(4-(dimethylamino)benzamide) (Compound 331)

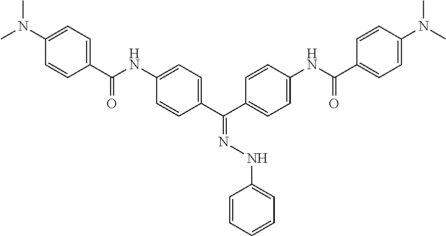

Compound 331 was prepared from compound 301 by a standard hydrazone synthesis procedure. [M+H]⁺ calcd for C₃₇H₃₆N₆O₂: 597.29. Found: 597.08.

### Example 232

### N,N′-(((methoxyimino)methylene)bis(4,1-phenylene))bis(4-((2-hydroxyethyl)(methyl)amino)benzamide) (Compound 332)

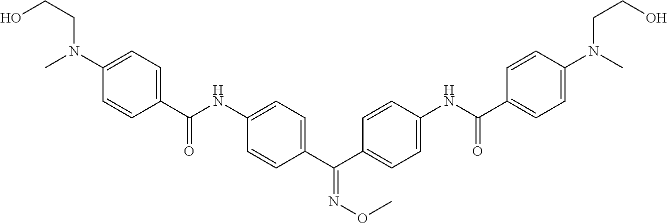 [SMILES: CON=C(c1ccc(NC(=O)c2ccc(N(C)CCO)cc2)cc1)c1ccc(NC(=O)c2ccc(N(C)CCO)cc2)cc1]

Compound 332 was prepared from compound 330 by a standard oxime synthesis procedure. [M+H]⁺ calcd for C₃₄H₃₇N₅O₅: 596.28. Found: 596.07.

### Example 233

### N,N′-(((methoxyimino)methylene)bis(4,1-phenylene))bis(4-fluorobenzamide) (Compound 333)

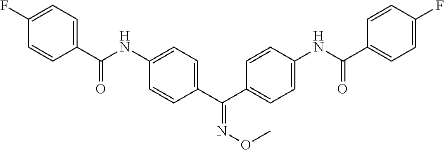 [SMILES: CON=C(c1ccc(NC(=O)c2ccc(F)cc2)cc1)c1ccc(NC(=O)c2ccc(F)cc2)cc1]

Compound 333 was prepared according to the procedure described in Scheme IV from 4,4′-diaminobenzophenone and 4-fluorobenzoate followed by a standard oxime synthesis procedure. ¹H NMR (500 MHz, DMSO-d₆) δ 11.00 (d, J=12.5 Hz, 1H), 10.41 (d, J=12.5 Hz, 1H), 8.03 (m, 4H), 7.85 (d, J=15 Hz, 2H), 7.79 (d, J=15 Hz, 2H), 7.38 (m, 6H), 7.29 (d, J=15 Hz, 2H), 3.79 (s, 3H).

### Example 234

### N,N′-(((methoxyimino)methylene)bis(4,1-phenylene))bis(4-(ethyl(2-hydroxyethyl)amino)benzamide) (Compound 334)

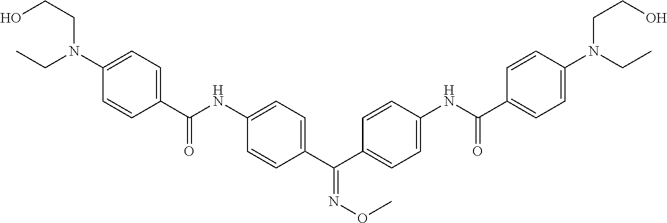 [SMILES: CCN(CCO)c1ccc(C(=O)Nc2ccc(C(=NOC)c3ccc(NC(=O)c4ccc(N(CC)CCO)cc4)cc3)cc2)cc1]

Compound 334 was prepared from compound 333 and N-2-hydroxyethyl-N-ethylamine by a standard procedure. [M+H]⁺ calcd for C₃₆H₄₁N₅O₅: 624.31. Found: 624.09.

### Example 235

### N,N′-(((methoxyimino)methylene)bis(4,1-phenylene))bis(4-((2-hydroxyethyl)amino)benzamide) (Compound 335)

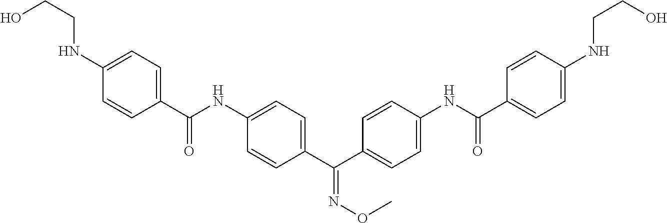 [SMILES: CON=C(c1ccc(NC(=O)c2ccc(NCCO)cc2)cc1)c1ccc(NC(=O)c2ccc(NCCO)cc2)cc1]

Compound 335 was prepared from compound 333 and 2-hydroxyethylamine by a standard procedure. [M+H]⁺ calcd for C₃₂H₃₃N₅O₅: 568.25. Found: 567.98.

### Example 236

### N,N′-(((methoxyimino)methylene)bis(4,1-phenylene))bis(4-(2-hydroxyethoxy)benzamide) (Compound 336)

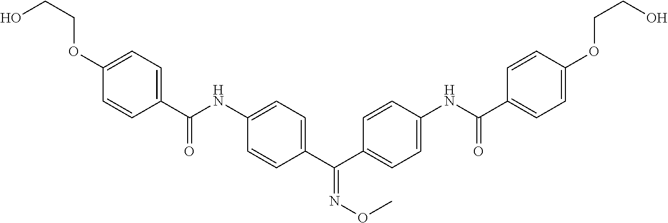 [SMILES: CON=C(c1ccc(NC(=O)c2ccc(OCCO)cc2)cc1)c1ccc(NC(=O)c2ccc(OCCO)cc2)cc1]

Compound 336 was prepared from compound 333 and ethylenediol by a standard procedure. [M+H]⁺ calcd for C₃₂H₃₁N₃O₇: 570.22. Found: 570.01.

### Example 237

### N,N′-(((methoxyimino)methylene)bis(4,1-phenylene))bis(4-(4-(3-hydroxypropyl)piperazin-1-yl)benzamide) (Compound 337)

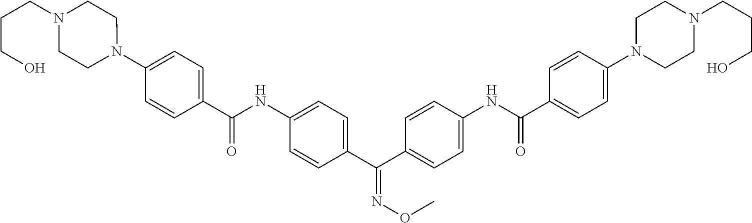 [SMILES: CON=C(c1ccc(NC(=O)c2ccc(N3CCN(CCCO)CC3)cc2)cc1)c1ccc(NC(=O)c2ccc(N3CCN(CCCO)CC3)cc2)cc1]

Compound 337 was prepared from compound 333 and 3-hydroxypropylpiperazine by a standard procedure. [M+H]⁺ calcd for C₄₂H₅₁N₇O₅: 734.93. Found: 734.23.

### Example 238

### N,N′-(((methoxyimino)methylene)bis(4,1-phenylene))bis(4-(4-(cyclopropanecarbonyl)piperazin-1-yl)benzamide) (Compound 338)

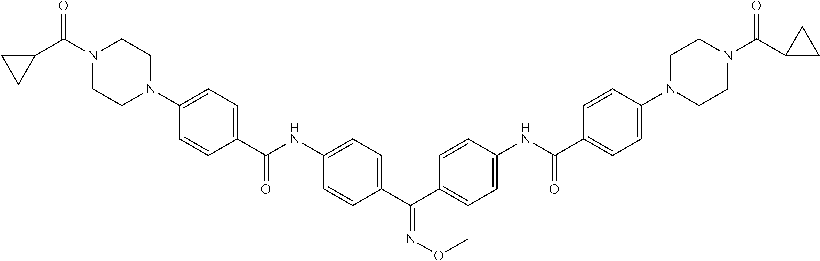 [SMILES: CON=C(c1ccc(NC(=O)c2ccc(N3CCN(C(=O)C4CC4)CC3)cc2)cc1)c1ccc(NC(=O)c2ccc(N3CCN(C(=O)C4CC4)CC3)cc2)cc1]

Compound 338 was prepared from compound 333 and 4-cyclopropylcarbonylpiperazine by a standard procedure. [M+H]⁺ calcd for C₄₄H₄₇N₇O₅: 754.92. Found: 754.29.

### Example 239

### N,N′-(((methoxyimino)methylene)bis(4,1-phenylene))bis(4-(piperidin-1-yl)benzamide) (Compound 339)

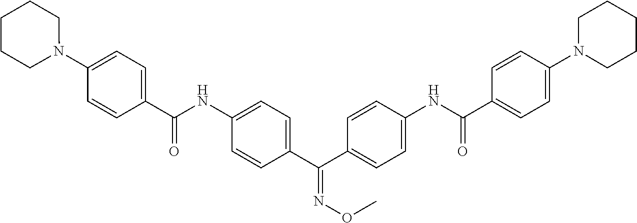 [SMILES: CON=C(c1ccc(NC(=O)c2ccc(N3CCCCC3)cc2)cc1)c1ccc(NC(=O)c2ccc(N3CCCCC3)cc2)cc1]

Compound 339 was prepared from compound 333 and piperidine by a standard procedure. [M+H]⁺ calcd for C₃₈H₄₁N₅O₃: 616.79. Found: 616.19.

### Example 240

### N,N′-(4,4′-(Oxazolidine-2,2-diyl)bis(4,1-phenylene))bis(4-(2-hydroxylethylamino)benzamide) (Compound 340)

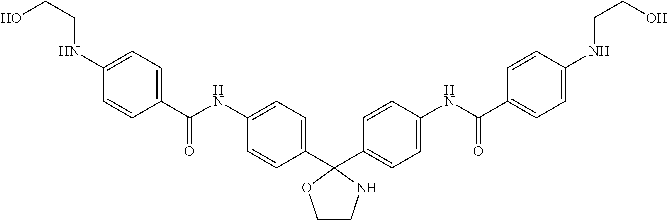 [SMILES: O=C(Nc1ccc(C2(c3ccc(NC(=O)c4ccc(NCCO)cc4)cc3)NCCO2)cc1)c1ccc(NCCO)cc1]

Compound 340 was prepared from N,N′-(4,4′-Benzophenone)bis(4-fluorobenzamide) and 2-hydroxyethylamine by a standard procedure. [M+H]⁺ calcd for C₃₃H₃₆N₅O₅: 582.27. Found: 582.07.

### Example 241

### 4,4′-Carbonylbis(4,1-phenylene)bis(4-(dimethylamino)benzoate (Compound 341)

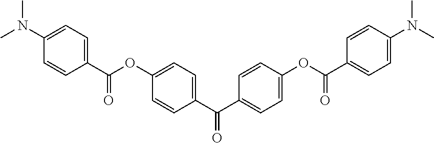

Compound 341 was prepared according to the procedure described in Scheme IV from 4,4′-dihydroxybenzophenone and 4-dimethylaminobenzoic acid. [M+H]⁺ calcd for C₃₁H₂₈N₂O₅: 509.20. Found: 509.05.

### Example 242

### N,N′-(4,4′-Carbonylbis(4,1-phenylene))bis(4-ethoxybenzamide) (Compound 342)

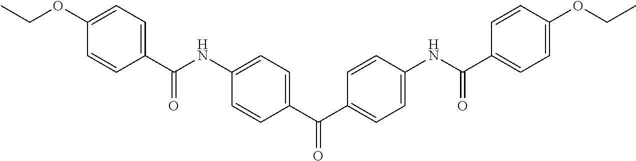 [SMILES: CCOc1ccc(C(=O)Nc2ccc(C(=O)c3ccc(NC(=O)c4ccc(OCC)cc4)cc3)cc2)cc1]

Compound 342 was prepared according to the procedure described in Scheme IV from 4,4′-diaminobenzophenone and 4-ethoxybenzoate. [M+H]⁺ calcd for C₃₁H₂₈N₂O₅: 509.20. Found: 508.98.

### Example 243

### N,N′-(4,4′-Carbonylbis(4,1-phenylene))bis(4-methoxybenzamide) (Compound 343)

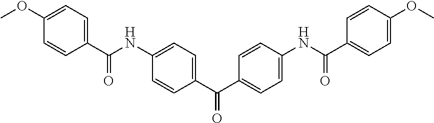

Compound 343 was prepared according to the procedure described in Scheme IV from 4,4′-diaminobenzophenone and 4-methoxybenzoate. [M+H]⁺ calcd for C₂₉H₂₄N₂O₅: 481.17. Found: 480.90.

### Example 244

### N,N′-(4,4′-Carbonylbis(4,1-phenylene))dibenzo[1,3]dioxole-5-carboxamide) (Compound 344)

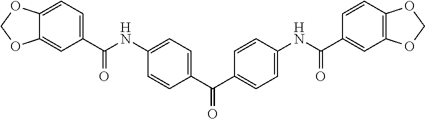 [SMILES: O=C(Nc1ccc(C(=O)c2ccc(NC(=O)c3ccc4c(c3)OCO4)cc2)cc1)c1ccc2c(c1)OCO2]

Compound 344 was prepared according to the procedure described in Scheme IV from 4,4′-diaminobenzophenone and benzo[1,3]dioxole-5-carboxylate. [M+H]⁺ calcd for C₂₉H₂₀N₂O₇: 509.13. Found: 508.91.

### Example 245

### N,N′-(4,4′-Carbonylbis(4,1-phenylene))bis(1H-indole-5-carboxamide) (Compound 345)

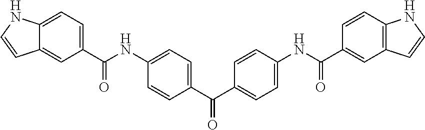 [SMILES: O=C(Nc1ccc(C(=O)c2ccc(NC(=O)c3ccc4[nH]ccc4c3)cc2)cc1)c1ccc2[nH]ccc2c1]

Compound 345 was prepared according to the procedure described in Scheme IV from 4,4′-diaminobenzophenone and 1H-indole-5-carboxylate. [M+H]⁺ calcd for C₃₁H₂₂N₄O₃: 499.17. Found: 498.92.

### Example 246

### N-(4-(4-Aminobenzoyl)phenyl)-1H-indole-5-carboxamide (Compound 346)

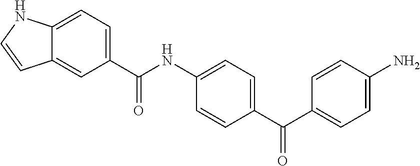 [SMILES: Nc1ccc(C(=O)c2ccc(NC(=O)c3ccc4[nH]ccc4c3)cc2)cc1]

Compound 346 was prepared according to the procedure described in Scheme IV from 4,4′-diaminobenzophenone and 1H-indole-5-carboxylate. [M+H]⁺ calcd for C₂₂H₁₇N₃O₂: 356.14. Found: 355.90.

### Example 247

### N,N′-(4,4′-Carbonylbis(4,1-phenylene))bis(4-(1H-pyrazol-1-yl)benzamide) (Compound 347)

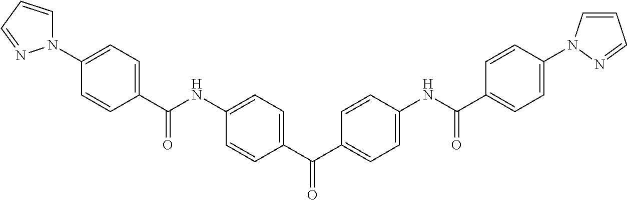 [SMILES: O=C(Nc1ccc(C(=O)c2ccc(NC(=O)c3ccc(-n4cccn4)cc3)cc2)cc1)c1ccc(-n2cccn2)cc1]

Compound 347 was prepared according to the procedure described in Scheme IV from 4,4′-diaminobenzophenone and 4-(pyrazol-1-yl)benzoate. [M+H]⁺ calcd for C₃₃H₂₄N₆O₃: 553.19. Found: 552.99.

### Example 248

### N-(4-(4-(2-phenylacetamido)benzoyl)phenyl)-1H-indole-5-carboxamide (Compound 348)

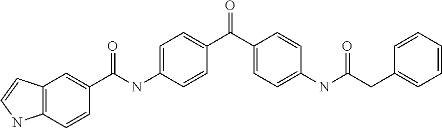 [SMILES: O=C(Cc1ccccc1)Nc1ccc(C(=O)c2ccc(NC(=O)c3ccc4[nH]ccc4c3)cc2)cc1]

Compound 348 was prepared from compound 346 and benzylbromide. [M+H]⁺ calcd for C₃₀H₂₃N₃O₃: 474.05. Found: 474.49.

### Example 249

### 4-ethoxy-N-(4-(4-(2-(4-ethoxyphenyl)-2-oxoethyl)benzoyl)phenyl)benzamide (Compound 349)

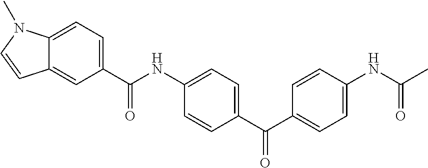 [SMILES: CC(=O)Nc1ccc(C(=O)c2ccc(NC(=O)c3ccc4c(ccn4C)c3)cc2)cc1]

Compound 349 was prepared according to the procedure described in Scheme IV from 4,4′-diaminobenzophenone and 1-methylindole-5-carboxylate. [M+H]⁺ calcd for C₂₅H₂₁N₃O₃: 412.07. Found: 411.94.

### Example 250

### N-(4-(4-(4-Methoxyphenylsulfonamido)benzoyl)phenyl)-4-(pyrrolidin-1-yl)benzamide (Compound 350)

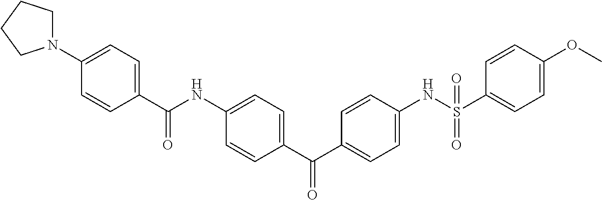 [SMILES: COc1ccc(S(=O)(=O)Nc2ccc(C(=O)c3ccc(NC(=O)c4ccc(N5CCCC5)cc4)cc3)cc2)cc1]

Compound 350 was prepared according to the procedure described in Scheme IV from 4,4′-diaminobenzophenone and 4-pyrrolidinylbenzoate. [M+H]⁺ calcd for C₃₁H₂₉N₃O₅S: 556.17. Found: 555.99.

### Example 251

### N,N′-(4,4′-Carbonylbis(4,1-phenylene))dithiophene-2-carboxamide (Compound 351)

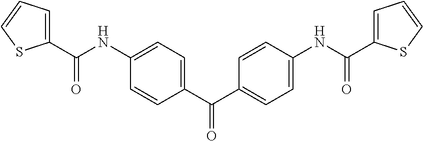 [SMILES: O=C(c1ccc(NC(=O)c2cccs2)cc1)c1ccc(NC(=O)c2cccs2)cc1]

Compound 351 was prepared according to the procedure described in Scheme IV from 4,4′-diaminobenzophenone and 2-thiophenecarboxylate. ¹H NMR (500 MHz, DMSO-d₆) δ 10.54 (s, 2H), 8.09 (d, J=3.75 Hz, 2H), 7.94 (d, J=12.5 Hz, 4H), 7.91 (d, J=3.75 Hz, 2H), 7.88 (d, J=12.5 Hz, 4H), 7.25 (t, J=3.75 Hz, 2H).

### Example 252

### N,N′-(4,4′-Carbonylbis(4,1-phenylene))bis(4-(2-hydroxyethoxy)benzamide) (Compound 352)

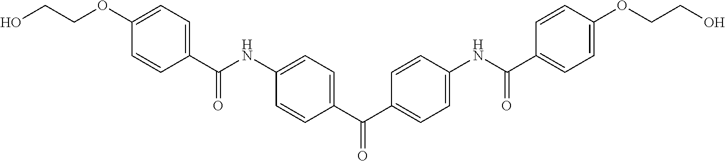 [SMILES: O=C(Nc1ccc(C(=O)c2ccc(NC(=O)c3ccc(OCCO)cc3)cc2)cc1)c1ccc(OCCO)cc1]

Compound 352 was prepared according to the procedure described in Scheme IV from 4,4′-diaminobenzophenone and 4-(2-hydroxyethoxy)benzoate. [M+H]⁺ calcd for C₃₁H₂₈N₂O₇: 541.09. Found: 541.05.

### Example 253

### N-(4-Benzoylphenyl)-4-(4-cyclopropanecarbonylpiperazin-1-yl)benzamide (Compound 353)

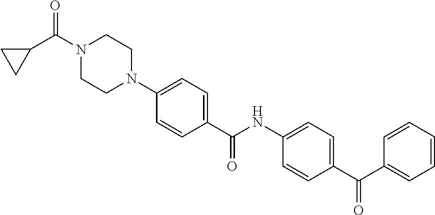 [SMILES: O=C(Nc1ccc(C(=O)c2ccccc2)cc1)c1ccc(N2CCN(C(=O)C3CC3)CC2)cc1]

Compound 353 was prepared according to the procedure described in Scheme IV from 4-aminobenzophenone and 4-piperazinebenzoate. [M+H]⁺ calcd for C₂₈H₂₈N₃O₃: 454.21. Found: 454.01.

### Example 254

### 4-(Dimethylamino)-N-(4-(4-(4-fluorobenzamido)benzoyl)phenyl)benzamide (Compound 354)

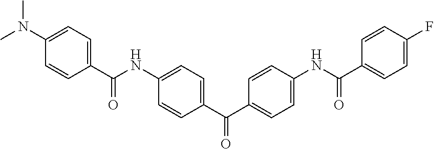 [SMILES: CN(C)c1ccc(C(=O)Nc2ccc(C(=O)c3ccc(NC(=O)c4ccc(F)cc4)cc3)cc2)cc1]

Compound 354 was prepared according to the procedure described in Scheme IV from 4,4′-diaminobenzophenone and the substituted benzoates. [M+H]⁺ calcd for C₂₉H₂₅FN₃O₃: 482.19. Found: 482.22.

### Example 255

### N,N′-(4,4′-Carbonylbis(4,1-phenylene))diisonicotinamide (Compound 355)

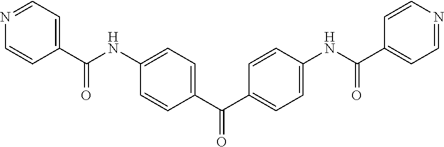 [SMILES: O=C(Nc1ccc(C(=O)c2ccc(NC(=O)c3ccncc3)cc2)cc1)c1ccncc1]

Compound 355 was prepared according to the procedure described in Scheme IV from 4,4′-diaminobenzophenone and 4-picolinic acid. [M+H]⁺ calcd for C₂₅H₁₉N₄O₃: 423.15. Found: 422.87.

### Example 256

### N,N′-(4,4′-Carbonylbis(4,1-phenylene))bis(6-morpholinonicotinamide) (Compound 356)

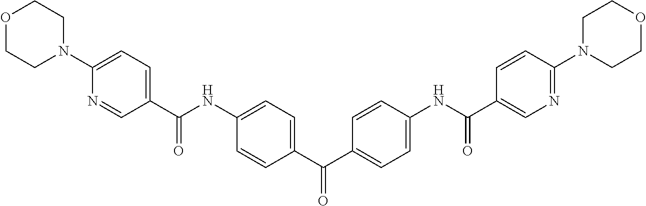 [SMILES: O=C(Nc1ccc(C(=O)c2ccc(NC(=O)c3ccc(N4CCOCC4)nc3)cc2)cc1)c1ccc(N2CCOCC2)nc1]

Compound 356 was prepared according to the procedure described in Scheme IV from 4,4′-diaminobenzophenone and 6-morpholinonicotinic acid. [M+H]⁺ calcd for C₃₃H₃₃N₆O₅: 593.25. Found: 593.03.

### Example 257

### N,N′-(4,4′-Carbonylbis(4,1-phenylene))bis(4-(morpholinomethyl)benzamide) (Compound 357)

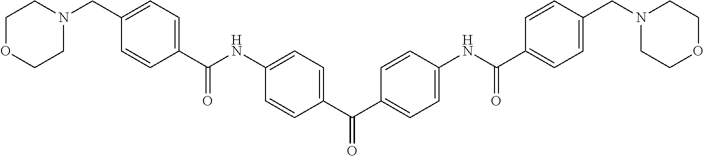 [SMILES: O=C(Nc1ccc(C(=O)c2ccc(NC(=O)c3ccc(CN4CCOCC4)cc3)cc2)cc1)c1ccc(CN2CCOCC2)cc1]

Compound 357 was prepared according to the procedure described in Scheme IV from 4,4′-diaminobenzophenone and 4-morpholinomethylbenzoate. [M+H]⁺ calcd for C₃₇H₃₉N₄O₅: 619.29. Found: 619.10.

### Example 258

### N-(4-(4-(4-Dimethylamino)benzoyl)phenyl)isonicotinamide (Compound 358)

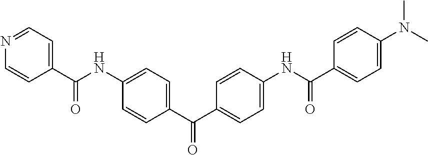 [SMILES: CN(C)c1ccc(C(=O)Nc2ccc(C(=O)c3ccc(NC(=O)c4ccncc4)cc3)cc2)cc1]

Compound 358 was prepared according to the procedure described in Scheme IV from 4,4′-diaminobenzophenone and 4-picolinic acid. [M+H]⁺ calcd for C₂₈H₂₅N₄O₃: 465.19. Found: 464.98.

### Example 259

### N-(4-(4-Cyclopropanecarboxamido)benzoyl)phenyl)-4-morpholinobenzamide (Compound 359)

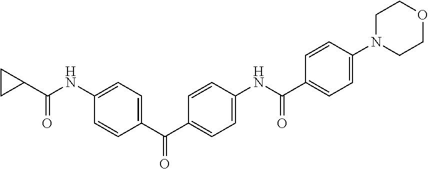 [SMILES: O=C(Nc1ccc(C(=O)c2ccc(NC(=O)C3CC3)cc2)cc1)c1ccc(N2CCOCC2)cc1]

Compound 359 was prepared according to the procedure described in Scheme IV from 4,4′-diaminobenzophenone and 4-morpholinobenzoate. [M+H]⁺ calcd for C₂₈H₂₈N₃O₄: 470.21. Found: 469.97.

### Example 260

### N-(4-(4-(2-Thienyl)carboxamido)benzoyl)phenyl)-4-morpholinobenzamide (Compound 360)

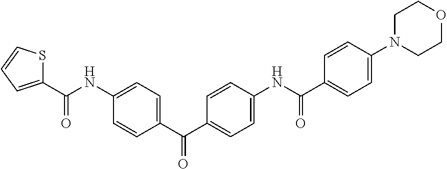 [SMILES: O=C(Nc1ccc(C(=O)c2ccc(NC(=O)c3cccs3)cc2)cc1)c1ccc(N2CCOCC2)cc1]

Compound 360 was prepared according to the procedure described in Scheme IV from 4,4′-diaminobenzophenone and 4-morpholinobenzoate. [M+H]⁺ calcd for C₂₉H₂₆N₃O₄S: 512.16. Found: 511.95.

### Example 261

### N,N′-(4,4′-Azanediylbis(4,1-phenylene))bis(4-(dimethylamino)benzamide) (Compound 361)

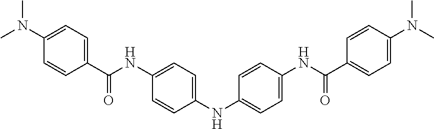

Compound 361 was prepared according to the procedure described in Scheme IV from 4,4′-diaminodiphenylamine and 4-dimethylaminobenzoate. [M+H]calcd for C₃₀H₃₁N₅O₂: 494.25. Found: 494.04.

### Example 262

### 4-(dimethylamino)-N-(4-(N-(4-(4-(dimethylamino)benzamido)phenyl)acetamido)phenyl)benzamide (Compound 362)

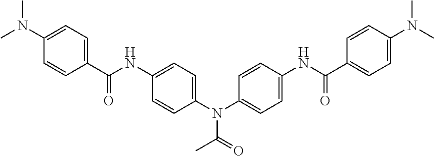

Compound 362 was prepared according to the procedure described in Scheme IV from 4,4′-diaminodiphenylamine and 4-dimethylaminobenzoate. ¹H NMR (500 MHz, DMSO-d₆) δ 9.99 (bs, 1H), 9.89 (bs, 1H), 7.84 (d, J=9 Hz, 6H), 7.70 (bs, 2H), 7.36 (bs, 2H), 7.20 (bs, 2H), 6.73 (d, J=9 Hz, 4H), 2.98 (s, 12H), 1.93 (s, 3H).

### Example 263

### N,N′-(4,4′-Azanediylbis(4,1-phenylene))bis(4-morpholinobenzamide) (Compound 363)

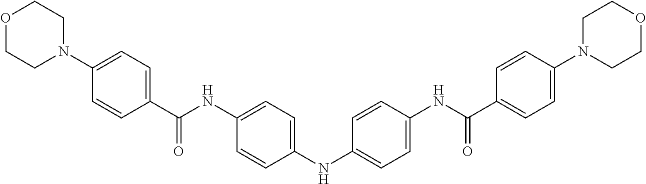 [SMILES: O=C(Nc1ccc(Nc2ccc(NC(=O)c3ccc(N4CCOCC4)cc3)cc2)cc1)c1ccc(N2CCOCC2)cc1]

Compound 363 was prepared according to the procedure described in Scheme IV from 4,4′-diaminodiphenylamine and 4-morpholinobenzoate. [M+H]⁺ calcd for C₃₄H₃₅N₅O₄: 578.27. Found: 578.11.

### Example 264

### N,N′-(4,4′-Azanediylbis(4,1-phenylene))bis(4-(4-hydroxypiperidino)benzamide) (Compound 364)

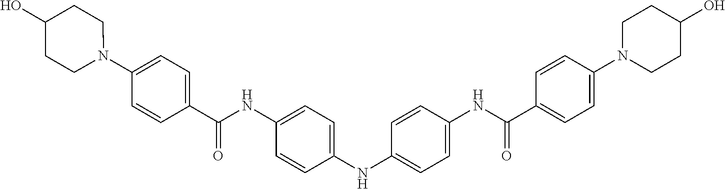 [SMILES: O=C(Nc1ccc(Nc2ccc(NC(=O)c3ccc(N4CCC(O)CC4)cc3)cc2)cc1)c1ccc(N2CCC(O)CC2)cc1]

Compound 364 was prepared according to the procedure described in Scheme IV from 4,4′-diaminodiphenylamine and 4-(4-hydroxypiperidinobenzoate. ¹H NMR (500 MHz, DMSO-d₆) δ 9.75 (s, 2H), 7.94 (s, 1H), 7.82 (d, J=9 Hz, 4H), 7.57 (d, J=9 Hz, 4H), 6.98 (t, J=9 Hz, 8H), 4.71 (d, J=4.5 Hz, 2H), 3.67 (m, 8H), 2.98 (dt, J=3, 10 Hz, 2H), 1.80 (m, 4H), 1.43 (m, 4H).

### Example 265

### N,N′-(4,4′-Azanediylbis(4,1-phenylene))bis(4-piperazinobenzamide) (Compound 365)

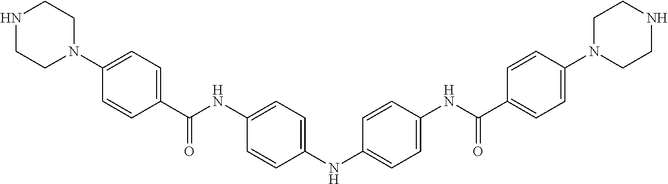 [SMILES: O=C(Nc1ccc(Nc2ccc(NC(=O)c3ccc(N4CCNCC4)cc3)cc2)cc1)c1ccc(N2CCNCC2)cc1]

Compound 365 was prepared according to the procedure described in Scheme IV from 4,4′-diaminodiphenylamine and 4-piperazinobenzoate. [M+H]⁺ calcd for C₃₄H₃₈N₇O₂: 576.31. Found: 576.17.

### Example 266

### 4-morpholino-N-(4-((4-(4-(piperazin-1-yl)benzamido)phenyl)amino)phenyl)benzamide (Compound 366)

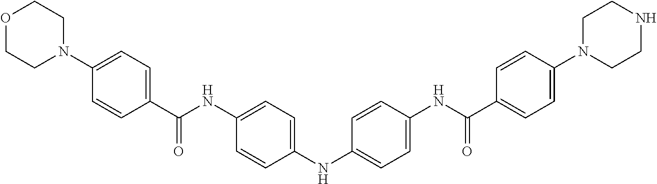 [SMILES: O=C(Nc1ccc(Nc2ccc(NC(=O)c3ccc(N4CCOCC4)cc3)cc2)cc1)c1ccc(N2CCNCC2)cc1]

Compound 366 was prepared according to the procedure described in Scheme IV from 4,4′-diaminodiphenylamine and 4-morpholinobenzoate. [M+H]⁺ calcd for C₃₄H₃₆N₆O₃: 577.21. Found: 577.16.

### Example 267

### 4-Fluoro-N-(4-(4-(4-piperazinobenzamido)phenylamino)phenyl)benzamide (Compound 367)

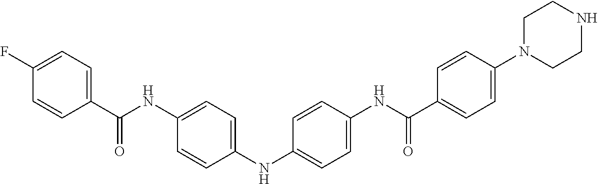 [SMILES: O=C(Nc1ccc(Nc2ccc(NC(=O)c3ccc(N4CCNCC4)cc3)cc2)cc1)c1ccc(F)cc1]

Compound 367 was prepared according to the procedure described in Scheme IV from 4,4′-diaminodiphenylamine and 4-piperazinobenzoate. [M+H]⁺ calcd for C₃₀H₂₉FN₅O₂: 510.23. Found: 510.06.

### Example 268

### N,N′-(4,4′-Azanediylbis(4,1-phenylene))bis(4-fluorobenzamide) (Compound 368)

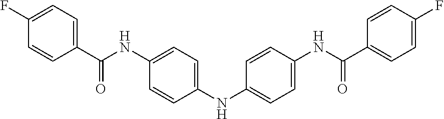 [SMILES: O=C(Nc1ccc(Nc2ccc(NC(=O)c3ccc(F)cc3)cc2)cc1)c1ccc(F)cc1]

Compound 368 was prepared according to the procedure described in Scheme IV from 4,4′-diaminodiphenylamine and 4-fluorobenzoate. [M+H]⁺ calcd for C₂₆H₁₉F2N₃O₂: 444.14. Found: 443.86.

### Example 269

### N-(1-(4-Aminophenyl)-1H-indol-5-yl)-4-dimethylaminobenzamide (Compound 369)

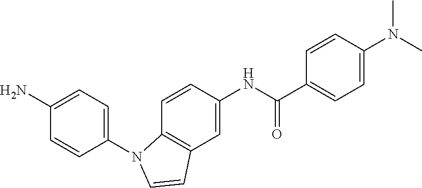 [SMILES: CN(C)c1ccc(C(=O)Nc2ccc3c(ccn3-c3ccc(N)cc3)c2)cc1]

Compound 369 was prepared according to the general procedure described in Scheme IV. Preparation of 4-(dimethylamino)-N-(1H-indol-5-yl)benzamide: 1H-indol-5-amine (200 mg, 1.51 mmol), hydroxybenzotriazole (204 mg, 1.51 mmol), triethylamine (0.23 mL, 1.66 mmol), and 4-(dimethylamino)benzoic acid (275 mg, 1.66 mmol) were taken up in DMF (7.5 mL) and stirred. EDC (319 mg, 1.66 mmol) was added to the solution last. After the addition, the solution was stirred at room temperature for 4 h. Water was then added to the solution and stirred for 10 min. The formed precipitate was filtered and washed well with water, followed by hexanes. The grayish solid was dried under vacuum to give 393 mg (93%) of the product.

Preparation of 4-(dimethylamino)-N-(1-(4-nitrophenyl)-1H-indol-5-yl)benzamide: 4-(Dimethylamino)-N-(1H-indol-5-yl)benzamide (200 mg, 0.71 mmol), 4-fluoronitrobenze (101 mg, 0.71 mmol) and potassium carbonate (99 mg, 0.72 mmol) were taken up in DMSO (7.2 mL). The solution was heated to 100° C. and stirred for 24 h. After the solution was cooled it was diluted with water until a precipitate formed and stirred well for 5 min. Filtration gave a yellow solid, which was then washed well with water, followed by hexanes. The solid was dried under vacuum to give 258 mg (90%) product as a yellow solid.

Preparation of Compound 369: 4-(Dimethylamino)-N-(1-(4-nitrophenyl)-1H-indol-5-yl)benzamide (330 mg, 0.82 mmol) was taken up in ethanol (28 mL) under nitrogen. The solution was treated with Pd(OH)₂ (35 mg, 0.24 mmol) and placed under a balloon of H₂ gas. After stirring at RT for 2 h, the catalyst was removed via filtration through celite. Concentration of the filtrate yielded 260 mg (85%) of compound 369. MS [M+H]⁺ calcd for C₂₃H₂₂N₄O: 371.18. Found: 370.94.

### Example 270

### N-(1-(4-(4-Dimethylaminobenzamido)phenyl)-1H-indol-5-yl)-4-dimethylaminobenzamide (Compound 370)

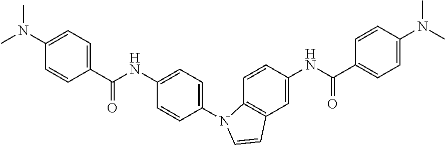

Compound 370 was prepared according to the procedure described in Scheme IV from 5-amino-1-(4-aminophenyl)indole and 4-dimethylaminobenzoate. [M+H]⁺ calcd for C₃₂H₃₁N₅O₂: 518.25. Found: 517.95.

### Example 271

### N-(1-(4-Aminophenyl)-1H-indazol-5-yl)-4-dimethylaminobenzamide (Compound 371)

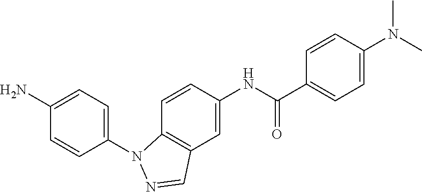 [SMILES: CN(C)c1ccc(C(=O)Nc2ccc3c(cnn3-c3ccc(N)cc3)c2)cc1]

Compound 371 was prepared according to the general procedure described in Scheme IV. Preparation of 4-(dimethylamino)-N-(1H-indazol-5-yl)benzamide: 1H-Indazol-5-amine (200 mg, 1.50 mmol), hydroxybenzotriazole (20 mg, 0.15 mmol), triethylamine (0.23 mL, 1.65 mmol), and 4-(dimethylamino)benzoic acid (273 mg, 1.65 mmol) were taken up in DMF (7.5 mL) and stirred. EDC (317 mg, 1.65 mmol) was added to the solution last. After the addition, the solution was stirred at room temperature for 4 h. Water was then added to the solution and stirred for 10 min. The formed precipitate was filtered and washed well with water, followed by hexanes. The solid was dried under vacuum to give 410 mg (97%) of the product as a brown solid.

Preparation of 4-(dimethylamino)-N-(1-(4-nitrophenyl)-1H-indazol-5-yl)benzamide: 4-(dimethylamino)-N-(1H-indazol-5-yl)benzamide (100 mg, 0.35 mmol), 4-fluoronitrobenze (50 mg, 0.35 mmol) and potassium carbonate (49 mg, 0.35 mmol) were taken up in DMSO (3.6 mL). The solution was heated to 100° C. and stirred for 24 h. After the solution was cooled it was diluted with water until a precipitate formed and stirred well for 5 min. Filtration gave a yellow solid, which was then washed well with water, followed by hexanes. The solid was dried under vacuum to give 133 mg (93%) of the product as a yellow solid.

Preparation of Compound 371: 4-(Dimethylamino)-N-(1-(4-nitrophenyl)-1H-indazol-5-yl)benzamide (400 mg, 0.99 mmol) was taken up in ethanol (33 mL) under nitrogen. The solution was treated with Pd(OH)₂ (35 mg, 0.25 mmol) and placed under a balloon of H₂ gas. After stirring at RT for 2 h, the catalyst was removed via filtration through celite. The filtrate was concentrated onto silica under reduced pressure. Purification via flash chromatography (0-5% MeOH/CH₂Cl₂) gave 180 mg (48%) of final compound 371 as a light brown solid. ¹H NMR (500 MHz, DMSO-d₆) 9.95 (s, 1H), 8.29 (s, 1H), 8.18 (s, 1H), 7.89 (d, J=9 Hz, 2H), 7.69 (dd, J=2, 9 Hz, 1H), 7.58 (d, J=9 Hz, 1H), 7.32 (d, J=9 Hz, 2H), 6.76 (d, J=9 Hz, 2H), 6.71 (d, J=9 Hz, 2H), 5.32 (s, 2H), 2.99 (s, 6H).

### Example 272

### N-(2-Acetyl-1-(4-(4-dimethylaminobenzamido)phenyl)-1H-indol-5-yl)-4-dimethylaminobenzamide (Compound 372)

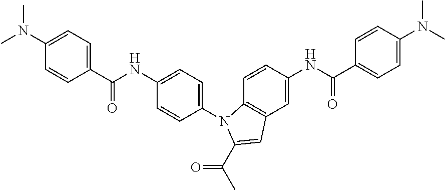

Compound 372 was prepared according to the procedure described in Scheme IV from 2-acetyl-5-amino-1-(4-aminophenyl)indole and 4-dimethylaminobenzoate. [M+H]⁺ calcd for C₃₄H₃₃N₅O₃: 560.26. Found: 560.02.

### Example 273

### N-(1-(4-(4-Dimethylaminobenzamido)phenyl)-1H-indazol-5-yl)-4-dimethylaminobenzamide (Compound 373)

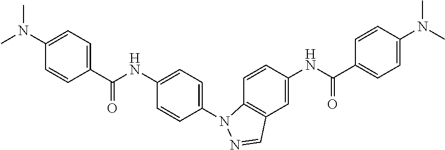

Compound 373 was prepared according to the procedure described in Scheme IV from 5-amino-1-(4-aminophenyl)indazole and 4-dimethylaminobenzoate. ¹H NMR (500 MHz, Acetone-d₆) δ 9.44 (s, 1H), 9.38 (s, 1H), 8.49 (s, 1H), 8.23 (s, 1H), 8.08 (d, J=9 Hz, 2H), 7.95 (d, J=9 Hz, 4H), 7.81 (m, 2H), 7.76 (d, J=9 Hz, 2H), 6.79 (d, J=9 Hz, 4H), 3.06 (s, 6H), 3.05 (s, 6H).

### Example 274

### N-(1-(4-Acetamidophenyl)-1H-indazol-5-yl)-4-dimethylaminobenzamide (Compound 374)

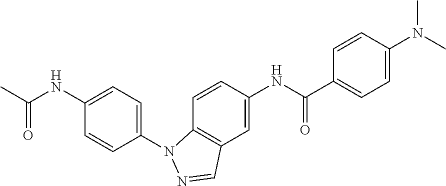 [SMILES: CC(=O)Nc1ccc(-n2ncc3cc(NC(=O)c4ccc(N(C)C)cc4)ccc32)cc1]

Compound 374 was prepared according to the procedure described in Scheme IV from 5-amino-(4-aminophenyl)indazole and 4-dimethylaminobenzoate.

[M+H]⁺ calcd for C₂₄H₂₃N₅O₂: 414.19. Found: 413.97.

### Example 275

### N-(1-(4-Benzamidophenyl)-1H-indazol-5-yl)-4-dimethylaminobenzamide (Compound 375)

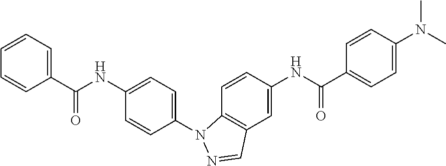 [SMILES: CN(C)c1ccc(C(=O)Nc2ccc3c(cnn3-c3ccc(NC(=O)c4ccccc4)cc3)c2)cc1]

Compound 375 was prepared according to the procedure described in Scheme IV from 5-amino-(4-aminophenyl)indazole and 4-dimethylaminobenzoate. ¹H NMR (500 MHz, DMSO-d₆) δ 10.45 (s, 1H), 10.01 (s, 1H), 8.37 (d, J=1.5 Hz, 1H), 8.33 (d, J=1 Hz, 1H), 8.01-7.78 (m, 11H), 7.55 (t, J=8 Hz, 2H), 6.77 (d, J=9 Hz, 2H), 3.00 (s, 6H).

### Example 276

### 4-(dimethylamino)-N-(1-(4-morpholinophenyl)-1H-indol-5-yl)benzamide (Compound 376)

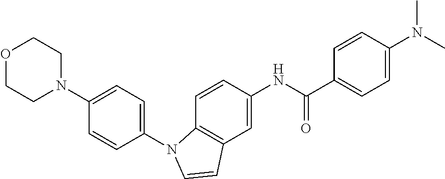 [SMILES: CN(C)c1ccc(C(=O)Nc2ccc3c(ccn3-c3ccc(N4CCOCC4)cc3)c2)cc1]

Compound 376 was prepared according to the procedure described in Scheme IV from 5-amino-1-(4-morpholinophenyl)indole and 4-dimethylaminobenzoate. [M+H]⁺ calcd for C₂₇H₂₈N₄O₂: 441.22. Found: 441.02.

### Example 277

### N-(1-(4-Morpholinocarboxamidophenyl)-1H-indazol-5-yl)-4-dimethylaminobenzamide (Compound 377)

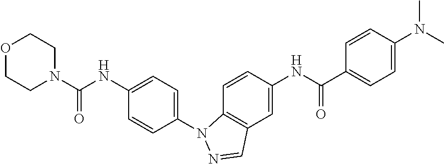 [SMILES: CN(C)c1ccc(C(=O)Nc2ccc3c(cnn3-c3ccc(NC(=O)N4CCOCC4)cc3)c2)cc1]

Compound 377 was prepared according to the procedure described in Scheme IV from 5-amino-1-(4-morpholinocarboxamidophenyl)indazole and 4-dimethylaminobenzoate. [M+H]⁺ calcd for C₂₇H₂₈N₆O₃: 485.22. Found: 485.02.

### Example 278

### N-(1-(4-Cyclopropanecarboxamidophenyl)-1H-indazol-5-yl)-4-dimethylaminobenzamide (Compound 378)

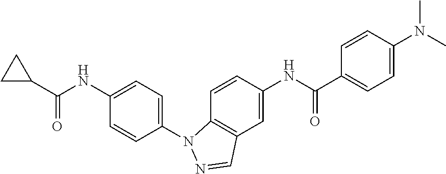 [SMILES: CN(C)c1ccc(C(=O)Nc2ccc3c(cnn3-c3ccc(NC(=O)C4CC4)cc3)c2)cc1]

Compound 378 was prepared according to the procedure described in Scheme IV from 5-amino-1-(4-aminophenyl)indazole and 4-dimethylaminobenzoate. ¹H NMR (500 MHz, DMSO-d₆) δ 10.38 (s, 1H), 10.00 (s, 1H), 8.35 (t, J=1 Hz, 1H), 8.30 (d, J=1 Hz, 1H), 7.89 (d, J=7 Hz, 2H), 7.80-7.76 (m, 4H), 7.68 (d, J=9 Hz, 2H), 6.76 (d, J=9 Hz, 2H), 3.00 (s, 6H), 1.80 (m, 1H), 0.82 (m, 4H).

### Example 279

### N-(2-Acetyl-1-(4-cyclopropanecarboxamidophenyl)-1H-indol-5-yl)-4-dimethylaminobenzamide (Compound 379)

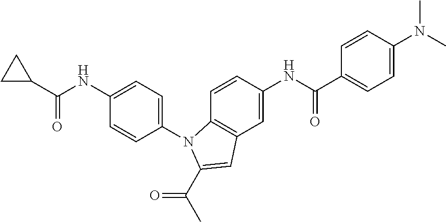 [SMILES: CC(=O)c1cc2cc(NC(=O)c3ccc(N(C)C)cc3)ccc2n1-c1ccc(NC(=O)C2CC2)cc1]

Compound 379 was prepared according to the procedure described in Scheme IV from 2-acetyl-5-amino-1-(4-aminophenyl)indole and 4-dimethylaminobenzoate. [M+H]⁺ calcd for C₂₉H₂₈N₄O₃: 481.22. Found: 480.97.

### Example 280

### N-(1-(4-Cyclopropanecarboxamidophenyl)-1H-indazol-5-yl)-4-morpholinobenzamide (Compound 380)

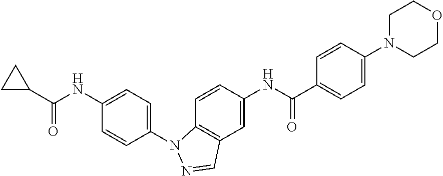 [SMILES: O=C(Nc1ccc2c(cnn2-c2ccc(NC(=O)C3CC3)cc2)c1)c1ccc(N2CCOCC2)cc1]

### Compound 380 was prepared according to the procedure described in Scheme IV from 5-amino-1-(4-aminophenyl)indazole and 4-morpholinobenzoate. [M+H]⁺ calcd for C₂₈H₂₇N₅O₃: 482.21. Found: 481.98.

### Example 281

### N-(1-(4-Aminophenyl)-1H-indazol-5-yl)-4-morpholinobenzamide (Compound 381)

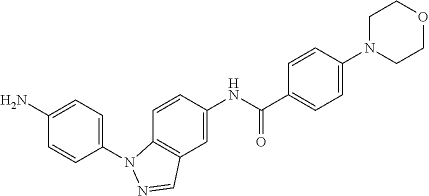 [SMILES: Nc1ccc(-n2ncc3cc(NC(=O)c4ccc(N5CCOCC5)cc4)ccc32)cc1]

Compound 381 was prepared according to the procedure described in Scheme IV from 5-amino-1-(4-aminophenyl)indazole and 4-morpholinobenzoate. [M+H]⁺ calcd for C₂₄H₂₃N₅O₂: 414.19. Found: 414.01.

### Example 282

### N-(1-(4-Pivaloylaminophenyl)-1H-indazol-5-yl)-4-morpholinobenzamide (Compound 382)

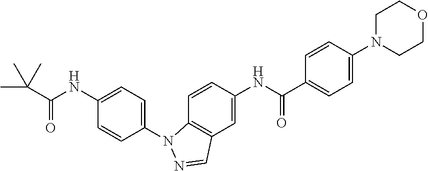 [SMILES: CC(C)(C)C(=O)Nc1ccc(-n2ncc3cc(NC(=O)c4ccc(N5CCOCC5)cc4)ccc32)cc1]

Compound 382 was prepared according to the procedure described in Scheme IV from 5-amino-1-(4-aminophenyl)indazole and 4-morpholinobenzoate. [M+H]⁺ calcd for C₂₉H₃₁N₅O₃: 498.24. Found: 498.05.

### Example 283

### tert-Butyl 1-(4-(4-dimethylaminobenzamido)phenyl)-1H-indazol-5-ylcarbamate (Compound 383)

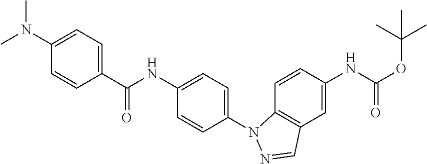 [SMILES: CN(C)c1ccc(C(=O)Nc2ccc(-n3ncc4cc(NC(=O)OC(C)(C)C)ccc43)cc2)cc1]

Compound 383 was prepared according to the procedure described in Scheme IV from 5-amino-1-(4-aminophenyl)indazole and 4-dimethylaminobenzoate. [M+H]⁺ calcd for C₂₇H₂₉N₅O₃: 472.23. Found: 472.06.

### Example 284

### N-(4-(5-(cyclopropanecarboxamido)-1H-indazol-1-yl)phenyl)-4-(dimethylamino)benzamide (Compound 384)

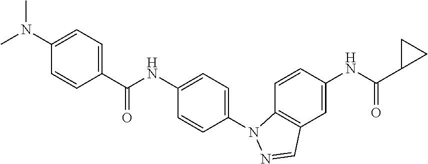 [SMILES: CN(C)c1ccc(C(=O)Nc2ccc(-n3ncc4cc(NC(=O)C5CC5)ccc43)cc2)cc1]

Compound 384 was prepared according to the procedure described in Scheme IV from 5-amino-1-(4-aminophenyl)indazole and 4-dimethylaminobenzoate. ¹H NMR (500 MHz, DMSO-d₆) δ 10.30 (s, 1H), 10.05 (s, 1H), 8.27 (d, J=1 Hz, 1H), 8.23 (s, 1H), 7.97 (d, J=9 Hz, 2H), 7.89 (d, J=9 Hz, 2H), 7.78 (d, J=9 Hz, 1H), 7.70 (d, J=9 Hz, 1H), 7.55 (dd, J=2, 12 Hz, 1H), 6.77 (d, J=9 Hz, 2H), 3.00 (s, 6H), 1.79 (m, 1H), 0.81 (m, 4H).

### Example 285

### N-(1-(4-(1-Cyanocyclopropanecarboxamido)phenyl)-1H-indazol-5-yl)-4-morpholinobenzamide (Compound 385)

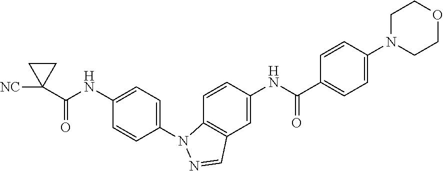 [SMILES: N#CC1(C(=O)Nc2ccc(-n3ncc4cc(NC(=O)c5ccc(N6CCOCC6)cc5)ccc43)cc2)CC1]

Compound 385 was prepared according to the procedure described in Scheme IV from 5-amino-1-(4-aminophenyl)indazole and 4-morpholinobenzoate. [M+H]⁺ calcd for C₂₉H₂₆N₆O₃: 507.21. Found: 507.01.

### Example 286

### N-(1-(4-Isobutyramidophenyl)-1H-indazol-5-yl)-4-morpholinobenzamide (Compound 386)

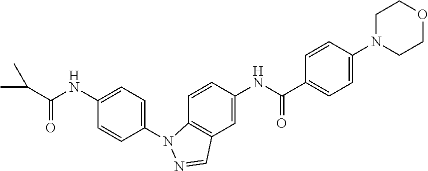 [SMILES: CC(C)C(=O)Nc1ccc(-n2ncc3cc(NC(=O)c4ccc(N5CCOCC5)cc4)ccc32)cc1]

Compound 386 was prepared according to the procedure described in Scheme IV from 5-amino-1-(4-aminophenyl)indazole and 4-morpholinobenzoate. [M+H]⁺ calcd for C₂₈H₂₉N₅O₃: 484.23. Found: 484.01.

### Example 287

### N-(1-(4-(1-Aminomethylcyclopropanecarboxamido)phenyl)-1H-indazol-5-yl)-4-morpholinobenzamide (Compound 387)

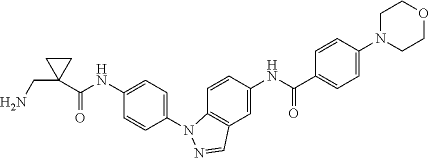 [SMILES: NCC1(C(=O)Nc2ccc(-n3ncc4cc(NC(=O)c5ccc(N6CCOCC6)cc5)ccc43)cc2)CC1]

Compound 387 was prepared by reduction of compound 385. [M+H]⁺ calcd for C₂₉H₃₀N₆O₃: 511.24. Found: 511.01.

### Example 288

### N-(1-(4-Amino-2-fluorophenyl)-1H-indazol-5-yl)-4-dimethylaminobenzamide (Compound 388)

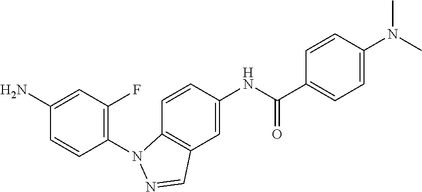 [SMILES: CN(C)c1ccc(C(=O)Nc2ccc3c(cnn3-c3ccc(N)cc3F)c2)cc1]

Compound 388 was prepared according to the procedure described in Scheme IV from 5-amino-1-(4-amino-2-fluorophenyl)indazole and 4-dimethylaminobenzoate. [M+H]⁺ calcd for C₂₂H₂₀FN₅O: 390.17. Found: 389.96.

### Example 289

### N-(1-(4-Cyclopropanecarboxamido-2-fluorophenyl)-1H-indazol-5-yl)-4-dimethylaminobenzamide (Compound 389)

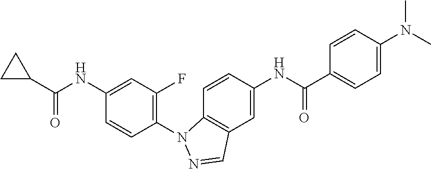 [SMILES: CN(C)c1ccc(C(=O)Nc2ccc3c(cnn3-c3ccc(NC(=O)C4CC4)cc3F)c2)cc1]

Compound 389 was prepared according to the procedure described in Scheme IV from 5-amino-1-(4-amino-2-fluorophenyl)indazole and 4-dimethylaminobenzoate. [M+H]⁺ calcd for C₂₆H₂₄FN₅O₂: 458.19. Found: 457.98.

### Example 290

### N-(1-(4-Acrylamidophenyl)-1H-indazol-5-yl)-4-morpholinobenzamide (Compound 390)

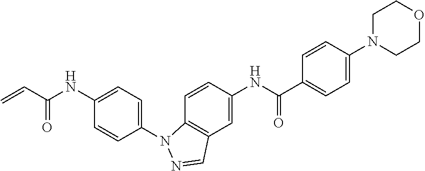 [SMILES: C=CC(=O)Nc1ccc(-n2ncc3cc(NC(=O)c4ccc(N5CCOCC5)cc4)ccc32)cc1]

Compound 390 was prepared according to the procedure described in Scheme IV from 5-amino-(4-aminophenyl)indazole and 4-morpholinobenzoate. ¹H NMR (500 MHz, Acetone-d₆) δ 9.59 (s, 1H), 9.47 (s, 1H), 8.49 (s, 1H), 8.24 (s, 1H), 7.98 (dd, J=2, 9 Hz, 4H), 7.81 (m, 2H), 7.77 (d, J=9 Hz, 1H), 7.05 (d, J=9 Hz, 2H), 6.52-6.38 (m, 2H), 5.75 (dd, J=2, 10 Hz, 1H), 3.80 (m, 4H), 3.29 (m, 4H).

### Example 291

### N-(1-(4-(3-Morpholinopropanamido)phenyl)-1H-indazol-5-yl)-4-morpholinobenzamide (Compound 391)

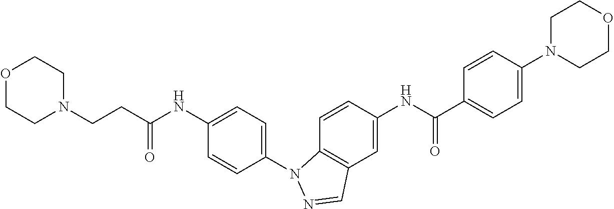 [SMILES: O=C(CCN1CCOCC1)Nc1ccc(-n2ncc3cc(NC(=O)c4ccc(N5CCOCC5)cc4)ccc32)cc1]

Compound 391 was prepared according to the procedure described in Scheme IV from 5-amino-1-(4-aminophenyl)indazole and 4-morpholinobenzoate. [M+H]⁺ calcd for C₃₁H₃₄N₆O₄: 555.26. Found: 555.09.

### Example 292

### N-(1-(4-(4-Dimethylaminobenzamido)-2-fluorophenyl)-1H-indazol-5-yl)-4-dimethylaminobenzamide (Compound 392)

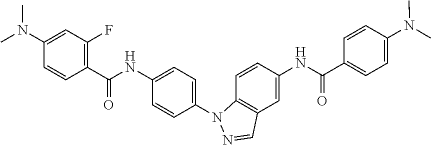

Compound 392 was prepared according to the procedure described in Scheme IV from 5-amino-(4-aminophenyl)indazole and 4-dimethylaminobenzoate. ¹H NMR (500 MHz, DMSO-d₆) δ 9.64 (s, 1H), 9.37 (s, 1H), 8.49 (m, 1H), 8.26 (s, 1H), 8.21 (m, 1H), 7.95 (d, J=9 Hz, 4H), 7.79 (dt, J=2, 9 Hz, 1H), 7.76 (m, 1H), 7.61 (t, J=9 Hz, 1H), 7.41 (dd, J=3, 9 Hz, 1H), 6.80 (m, 4H), 3.07 (s, 6H), 3.05 (s, 6H).

### Example 293

### N-(1-(4-(2-Thienyl)carboxaminophenyl)-1H-indazol-5-yl)-4-morpholinobenzamide (Compound 393)

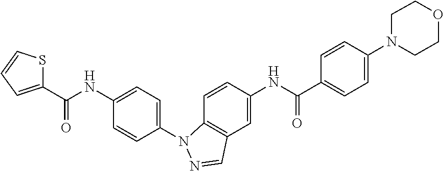 [SMILES: O=C(Nc1ccc2c(cnn2-c2ccc(NC(=O)c3cccs3)cc2)c1)c1ccc(N2CCOCC2)cc1]

Compound 393 was prepared according to the procedure described in Scheme IV from 5-amino-1-(4-aminophenyl)indazole and 4-morpholinobenzoate. [M+H]⁺ calcd for C₂₉H₂₅N₅O₃S: 524.17. Found: 523.97.

### Example 294

### N-(1-(4-Cyclobutanecarboxaminophenyl)-1H-indazol-5-yl)-4-morpholinobenzamide (Compound 394)

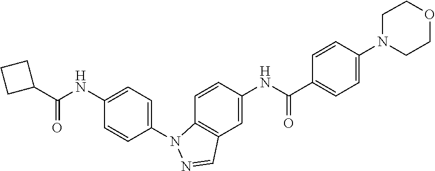 [SMILES: O=C(Nc1ccc2c(cnn2-c2ccc(NC(=O)C3CCC3)cc2)c1)c1ccc(N2CCOCC2)cc1]

Compound 394 was prepared according to the procedure described in Scheme IV from 5-amino-1-(4-aminophenyl)indazole and 4-morpholinobenzoate. [M+H]⁺ calcd for C₂₉H₂₉N₅O₃: 496.23. Found: 496.03.

### Example 295

### (±)-N-(1-(4-(2,2-Difluorocyclopropane)carboxamidophenyl)-1H-indazol-5-yl)-4-morpholinobenzamide (Compound 395)

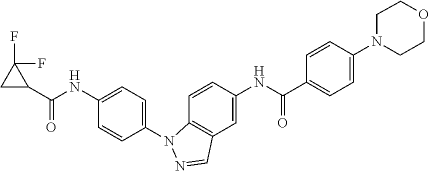 [SMILES: O=C(Nc1ccc2c(cnn2-c2ccc(NC(=O)C3CC3(F)F)cc2)c1)c1ccc(N2CCOCC2)cc1]

Compound 395 was prepared according to the procedure described in Scheme IV from 5-amino-1-(4-aminophenyl)indazole and 4-morpholinobenzoate. [M+H]⁺ calcd for C₂₈H₂₅F2N₅O₃: 518.19. Found: 518.03.

### Example 296

### N-(1-(4-(1-Trifluoromethylcyclopropane)carboxaminophenyl)-1H-indazol-5-yl)-4-morpholinobenzamide (Compound 396)

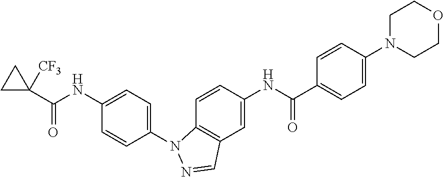 [SMILES: O=C(Nc1ccc2c(cnn2-c2ccc(NC(=O)C3(C(F)(F)F)CC3)cc2)c1)c1ccc(N2CCOCC2)cc1]

Compound 396 was prepared according to the procedure described in Scheme IV from 5-amino-1-(4-aminophenyl)indazole and 4-morpholinobenzoate. [M+H]⁺ calcd for C₂₉H₂₆F₃N₅O₃: 550.21. Found: 550.16.

### Example 297

### N-(1-(4-Cyclopropanecarboxamidophenyl)-1H-indazol-5-yl)-4-(4-hydroxypiperidino)benzamide (Compound 397)

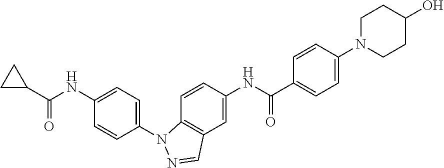 [SMILES: O=C(Nc1ccc2c(cnn2-c2ccc(NC(=O)C3CC3)cc2)c1)c1ccc(N2CCC(O)CC2)cc1]

Compound 397 was prepared according to the procedure described in Scheme IV from 5-amino-1-(4-aminophenyl)indazole and 4-(4-hydroxypiperidino)benzoate. [M+H]⁺ calcd for C₂₉H₂₉N₅O₃: 496.23. Found: 496.10.

### Example 298

### N-(1-(4-(3-Methylbut-2-enamido)phenyl)-1H-indazol-5-yl)-4-morpholinobenzamide (Compound 398)

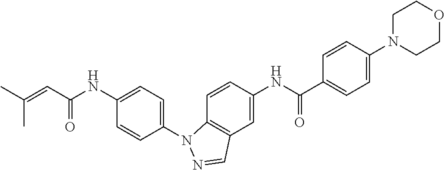 [SMILES: CC(C)=CC(=O)Nc1ccc(-n2ncc3cc(NC(=O)c4ccc(N5CCOCC5)cc4)ccc32)cc1]

Compound 398 was prepared according to the procedure described in Scheme IV from 5-amino-1-(4-aminophenyl)indazole and 4-morpholinobenzoate. ¹H NMR (500 MHz, Acetone-d₆) δ 9.47 (s, 1H), 9.27 (s, 1H), 8.48 (s, 1H), 8.23 (s, 1H), 7.98 (d, J=8.5 Hz, 2H), 7.94 (d, J=8.5 Hz, 2H), 7.80 (s, 2H), 7.73 (d, J=8.5 Hz, 2H), 7.05 (d, J=8.5 Hz, 2H), 5.92 (s, 1H), 3.80 (t, J=5 Hz, 4H), 3.29 (t, J=5 Hz, 4H), 2.24 (s, 3H), 1.90 (s, 3H).

### Example 299

### N-(1-(4-Cyclopropanecarboxamidophenyl)-1H-indazol-5-yl)-4-piperazinobenzamide (Compound 399)

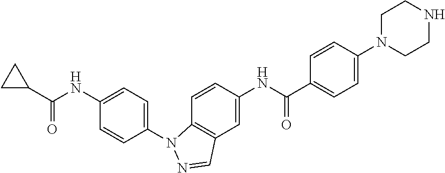 [SMILES: O=C(Nc1ccc2c(cnn2-c2ccc(NC(=O)C3CC3)cc2)c1)c1ccc(N2CCNCC2)cc1]

Compound 399 was prepared according to the procedure described in Scheme IV from 5-amino-1-(4-aminophenyl)indazole and 4-piperazinobenzoate. [M+H]⁺ calcd for C₂₈H₂₈N₆O₂: 481.23. Found: 481.04.

### Example 300

### N-(4-(4-(4-(4-Cyclopropanecarbonyl)piperazin-1-yl)benzamido)benzoyl)phenyl)thiophene-2-carboxamide (Compound 400)

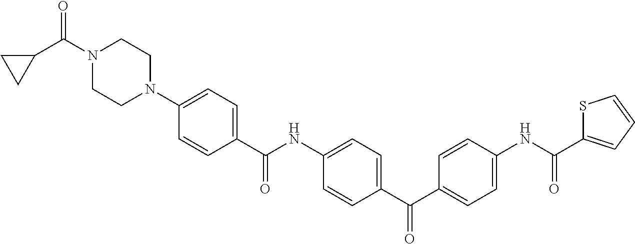 [SMILES: O=C(Nc1ccc(C(=O)c2ccc(NC(=O)c3cccs3)cc2)cc1)c1ccc(N2CCN(C(=O)C3CC3)CC2)cc1]

Compound 400 was prepared according to the procedure described in Scheme IV from 4,4′-diaminobenzophenone and 4-piperazinebenzoate. [M+H]⁺ calcd for C₃₃H₃₀N₄O₄S: 579.51. Found: 579.06.

### Example 301

### N-(4-(4-(4-(4-Cyclopropanecarbonyl)piperazin-1-yl)benzamido)benzoyl)phenyl)cyclopropanecarboxamide (Compound 401)

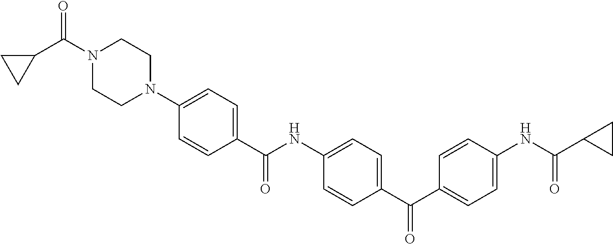 [SMILES: O=C(Nc1ccc(C(=O)c2ccc(NC(=O)C3CC3)cc2)cc1)c1ccc(N2CCN(C(=O)C3CC3)CC2)cc1]

Compound 401 was prepared according to the procedure described in Scheme IV from 4,4′-diaminobenzophenone and 4-piperazinebenzoate. [M+H]⁺ calcd for C₃₂H₃₂N₄O₄: 537.15. Found: 537.06.

### Example 302

### N-(4-(4-(4-Morpholinobenzamido)benzoyl)phenyl)-1H-indole-5-carboxamide (Compound 402)

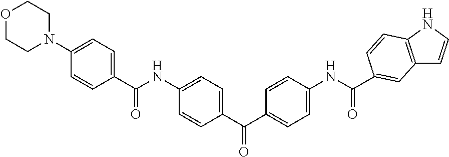 [SMILES: O=C(Nc1ccc(C(=O)c2ccc(NC(=O)c3ccc4[nH]ccc4c3)cc2)cc1)c1ccc(N2CCOCC2)cc1]

Compound 402 was prepared according to the procedure described in Scheme IV from 4,4′-diaminobenzophenone and 4-piperazinebenzoate. [M+H]⁺ calcd for C₃₃H₂₈N₄O₄: 545.13. Found: 545.03.

### Example 303

### N,N′-(carbonylbis(4,1-phenylene))bis(1-methyl-1H-benzo[d][1,2,3]triazole-5-carboxamide) (Compound 403)

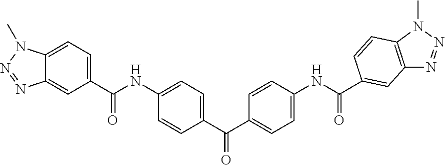 [SMILES: Cn1nnc2cc(C(=O)Nc3ccc(C(=O)c4ccc(NC(=O)c5ccc6c(c5)nnn6C)cc4)cc3)ccc21]

Compound 403 was prepared according to the procedure described in Scheme IV from 4,4′-diaminobenzophenone and 1-methyl-1H-benzotriazole-5-carboxylate. [M+H]⁺ calcd for C₂₉H₂₂N₈O₃: 531.06. Found: 531.05.

### Example 304

### N-(1-(4-Cyanophenyl)-1H-indazol-5-yl)-4-(4-hydroxypiperidino)benzamide (Compound 404)

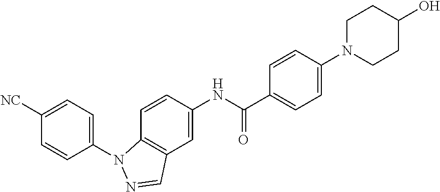 [SMILES: N#Cc1ccc(-n2ncc3cc(NC(=O)c4ccc(N5CCC(O)CC5)cc4)ccc32)cc1]

Compound 404 was prepared according to the procedure described in Scheme IV from 5-amino-1-(4-cyanophenyl)indazole and 4-(4-hydroxypiperidino)benzoate. [M+H]⁺ calcd for C₂₆H₂₃N₅O₂: 438.19. Found: 437.99.

### Example 305

### N-(1-(4-Cyanophenyl)-1H-indazol-5-yl)-4-morpholinobenzamide (Compound 405)

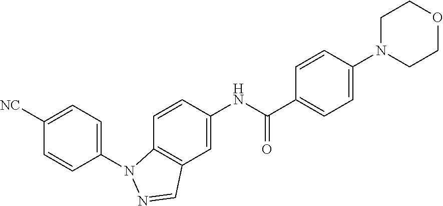 [SMILES: N#Cc1ccc(-n2ncc3cc(NC(=O)c4ccc(N5CCOCC5)cc4)ccc32)cc1]

Compound 405 was prepared according to the procedure described in Scheme IV from 5-amino-1-(4-cyanophenyl)indazole and 4-morpholinobenzoate. [M+H]⁺ calcd for C₂₅H₂₁N₅O₂: 424.17. Found: 424.09.

### Example 306

### 1-(4-(4-Dimethylaminobenzamido)phenyl)-N-(4-morpholinophenyl)-1H-indole-5-carboxamide (Compound 406)

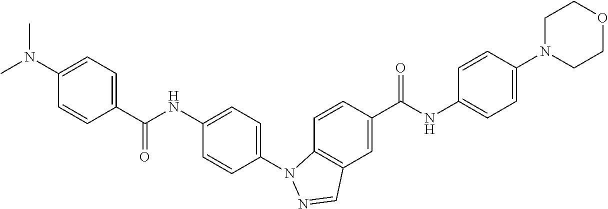 [SMILES: CN(C)c1ccc(C(=O)Nc2ccc(-n3ncc4cc(C(=O)Nc5ccc(N6CCOCC6)cc5)ccc43)cc2)cc1]

Compound 406 was prepared according to the procedure described in Scheme IV from 1-(4-dimethylaminophenylbenzamido)indole and 4-morpholinoaniline. ¹H NMR (500 MHz, DMSO-d₆) δ10.08 (s, 1H), 10.02 (2, 1H), 8.33 (d, J=2 Hz, 1H), 7.99 (d, J=9 Hz, 2H), 7.89 (d, J=9 Hz, 2H), 7.82 (dd, J=2, 9 Hz, 1H), 7.74 (d, J=3 Hz, 1H), 7.66 (d, J=9 Hz, 2H), 7.60 (d, J=9 Hz, 1H), 7.57 (d, J=5 Hz, 2H), 6.93 (d, J=9 Hz, 2H), 6.83 (d, J=3 Hz, 1H), 6.78 (d, J=9 Hz, 2H), 3.73 (m, 4H), 3.06 (m, 4H), 3.01 (s, 6H).

### Example 307

### 4-Dimethylamino-N-(1-(4-(4-dimethylaminobenzamido)benzoyl)piperidin-4-yl)benzamide (Compound 407)

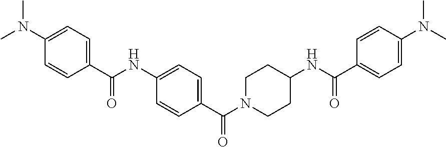

Compound 407 was prepared according to the procedure described in Scheme IV from 4-(4-dimethylaminobenzamido)benzoate and 4-(4-dimethylaminophenyl)piperidine. [M+H]⁺ calcd for C₃₀H₃₅N₅O₃: 514.27. Found: 513.98.

### Example 308

### N,N′-(4,4′-Thiobis(4,1-phenylene))bis(4-(dimethylamino)benzamide) (Compound 408)

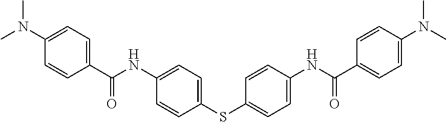

Compound 408 was prepared according to the procedure described in Scheme IV from 4,4′-diaminodiphenylamine and 4-dimethylaminobenzoate. [M+H]⁺ calcd for C₃₀H₃₀N₄O₂S: 511.21. Found: 510.94.

### Example 309

### tert-butyl (4-(4-(4-morpholinobenzamido)piperidine-1-carbonyl)phenyl)carbamate

(Compound 409)

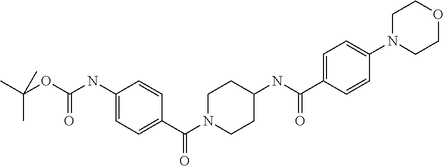 [SMILES: CC(C)(C)OC(=O)Nc1ccc(C(=O)N2CCC(NC(=O)c3ccc(N4CCOCC4)cc3)CC2)cc1]

Compound 409 was prepared according to the procedure described in Scheme IV from 4-aminobenzoate and 4-(4-dimethylaminophenyl)piperidine. [M+H]⁺ calcd for C₂₈H₃₆N₄O₅: 509.27. Found: 508.99.

### Example 310

### N-(4-(4-Morpholinophenylamino)phenyl)-4-dimethylaminobenzamide (Compound 410)

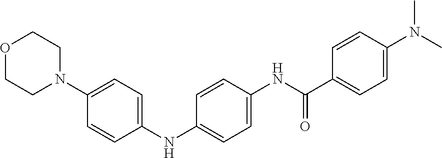 [SMILES: CN(C)c1ccc(C(=O)Nc2ccc(Nc3ccc(N4CCOCC4)cc3)cc2)cc1]

Compound 410 was prepared according to the procedure described in Scheme IV from 4,4′-diaminodiphenylamine and 4-dimethylaminobenzoate. [M+H]⁺ calcd for C₂₅H₂₉N₄O₂: 417.23. Found: 417.01.

### Example 311

### 4-Dimethylamino-N-(4-(2-(4-(4-hydroxypiperidinyl)phenyl)-1H-benzimidazol-5-ylcarbamoyl)phenyl)benzamide (Compound 411)

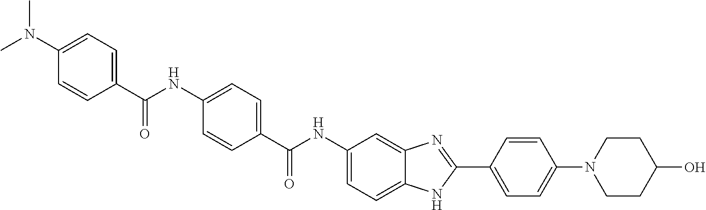 [SMILES: CN(C)c1ccc(C(=O)Nc2ccc(C(=O)Nc3ccc4[nH]c(-c5ccc(N6CCC(O)CC6)cc5)nc4c3)cc2)cc1]

Compound 411 was prepared according to the procedure similar to that described in Scheme III from 3,4-dinitro-N-(4-aminobenzoyl)aniline and 4-(4-hydroxypiperidinyl)benzaldehyde. [M+H]⁺ calcd for C₃₄H₃₄N₆O₃: 575.27. Found: 575.07.

### Example 312

### 4-Dimethylamino-N-(4-(2-(4-(bis(2-hydroxyethyl)amino)phenyl)-1H-benzimidazol-5-ylcarbamoyl)phenyl)benzamide (Compound 412)

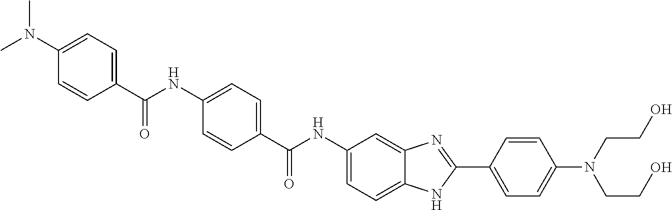 [SMILES: CN(C)c1ccc(C(=O)Nc2ccc(C(=O)Nc3ccc4[nH]c(-c5ccc(N(CCO)CCO)cc5)nc4c3)cc2)cc1]

Compound 412 was prepared according to the procedure similar to that described in Scheme III from 3,4-dinitro-N-(4-aminobenzoyl)aniline and 4-bis(2-hydroxyethyl)aminobenzaldehyde. [M+H]⁺ calcd for C₃₃H₃₄N₆O₄: 579.26. Found: 579.06.

### Example 313

### N-(2-(4-(1,4-Dioxa-8-azaspiro[4,5]decan-8-yl)phenyl)-1H-benzimidazol-5-yl)-4-morpholinobenzamide (Compound 413)

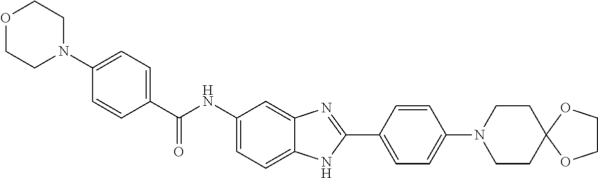 [SMILES: O=C(Nc1ccc2[nH]c(-c3ccc(N4CCC5(CC4)OCCO5)cc3)nc2c1)c1ccc(N2CCOCC2)cc1]

Compound 413 was prepared according to the procedure similar to that described in Scheme III from 3,4-dinitro-N-(4-morpholinobenzoyl)aniline and 4-(1,4-dioxa-8-azaspiro[4,5]decan-8-yl)benzaldehyde. [M+H]⁺ calcd for C₃₁H₃₃N₅O₄: 540.25. Found: 540.05.

### Example 314

### N-(2-(4-(4-(2-Hydroxyethyl)piperidinyl)phenyl)-1H-benzimidazol-5-yl)-4-morpholinobenzamide (Compound 414)

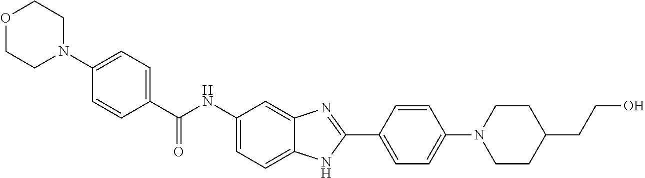 [SMILES: O=C(Nc1ccc2[nH]c(-c3ccc(N4CCC(CCO)CC4)cc3)nc2c1)c1ccc(N2CCOCC2)cc1]

Compound 414 was prepared according to the procedure similar to that described in Scheme III from 3,4-dinitro-N-(4-morpholinobenzoyl)aniline and 4-(2-hydroxyethyl)piperidinylbenzaldehyde. [M+H]⁺ calcd for C₃₁H₃₅N₅O₃: 526.27. Found: 526.11.

### Example 315

### N-(2-(4-(4-Cyclopropanecarbonylpiperazinyl)phenyl)-1H-benzimidazol-5-yl)-4-morpholinobenzamide (Compound 415)

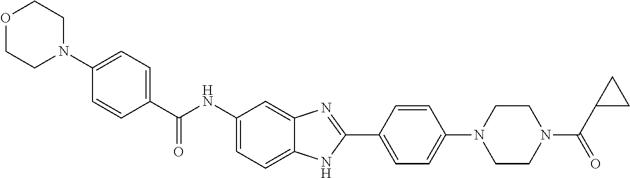 [SMILES: O=C(Nc1ccc2[nH]c(-c3ccc(N4CCN(C(=O)C5CC5)CC4)cc3)nc2c1)c1ccc(N2CCOCC2)cc1]

Compound 415 was prepared according to the procedure similar to that described in Scheme III from 3,4-dinitro-N-(4-morpholinobenzoyl)aniline and 4-(4-cyclopropanecarbonylpiperazinyl)benzaldehyde. [M+H]⁺ calcd for C₃₂H₃₄N₆O₃: 551.27. Found: 551.10.

### Example 316

### N-(2-(4-(4-(2-Hydroxymethyl)piperidinyl)phenyl)-1H-benzimidazol-5-yl)-4-morpholinobenzamide (Compound 416)

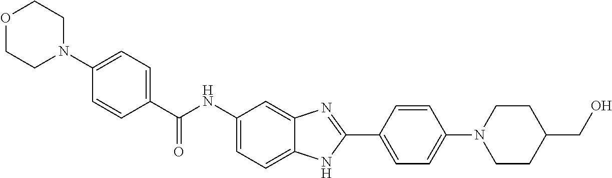 [SMILES: O=C(Nc1ccc2[nH]c(-c3ccc(N4CCC(CO)CC4)cc3)nc2c1)c1ccc(N2CCOCC2)cc1]

Compound 416 was prepared according to the procedure similar to that described in Scheme III from 3,4-dinitro-N-(4-morpholinobenzoyl)aniline and 4-(2-hydroxymethyl)piperidinylbenzaldehyde. [M+H]⁺ calcd for C₃₀H₃₃N₅O₃: 512.26. Found: 512.09.

### Example 317

### N-(2-(2-(4-Hydroxypiperidinyl)thien-5-yl)-1H-benzimidazol-5-yl)-4-morpholinobenzamide (Compound 417)

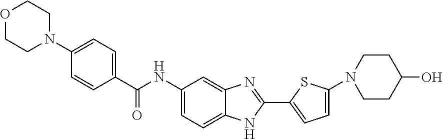 [SMILES: O=C(Nc1ccc2[nH]c(-c3ccc(N4CCC(O)CC4)s3)nc2c1)c1ccc(N2CCOCC2)cc1]

Compound 417 was prepared according to the procedure similar to that described in Scheme III from 3,4-dinitro-N-(4-morpholinobenzoyl)aniline and 5-(4-hydroxypiperidinyl)thiophene-2-carboxaldehyde. [M+H]⁺ calcd for C₂₇H₂₉N₅O₃S: 504.20. Found: 503.99.

### Example 318

### N-(2-(2-Morpholinothien-5-yl)-1H-benzimidazol-5-yl)-4-morpholinobenzamide (Compound 418)

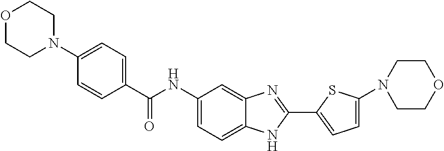 [SMILES: O=C(Nc1ccc2[nH]c(-c3ccc(N4CCOCC4)s3)nc2c1)c1ccc(N2CCOCC2)cc1]

Compound 418 was prepared according to the procedure similar to that described in Scheme III from 3,4-dinitro-N-(4-morpholinobenzoyl)aniline and 5-morpholinothiophene-2-carboxaldehyde. [M+H]⁺ calcd for C₂₆H₂₇N₅O₃S: 490.18. Found: 489.95.

### Example 319

### N-(2-(3-Morpholinomethylphenyl)-1H-benzimidazol-5-yl)-4-morpholinobenzamide (Compound 419)

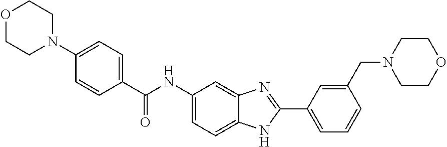 [SMILES: O=C(Nc1ccc2[nH]c(-c3cccc(CN4CCOCC4)c3)nc2c1)c1ccc(N2CCOCC2)cc1]

Compound 419 was prepared according to the procedure similar to that described in Scheme III from 3,4-dinitro-N-(4-morpholinobenzoyl)aniline and 3-morpholinomethylbenzaldehyde. [M+H]⁺ calcd for C₂₉H₃₁N₅O₃: 498.24. Found: 497.98.

### Example 320

### N-(2-(1-Methylpiperidin-4-yl)-1H-benzimidazol-5-yl)-4-morpholinobenzamide (Compound 420)

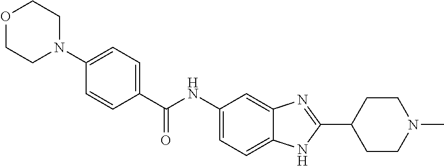 [SMILES: CN1CCC(c2nc3cc(NC(=O)c4ccc(N5CCOCC5)cc4)ccc3[nH]2)CC1]

Compound 420 was prepared according to the procedure similar to that described in Scheme III from 3,4-dinitro-N-(4-morpholinobenzoyl)aniline and 4-(1-methylpiperidinyl)caboxaldehyde. [M+H]⁺ calcd for C₂₄H₂₉N₅O₂: 420.23. Found: 420.04.

### Example 321

### N-(2-(4-(1,1-Dioxo-4-thiomorpholino)phenyl)-1H-benzimidazol-5-yl)-4-morpholinobenzamide (Compound 421)

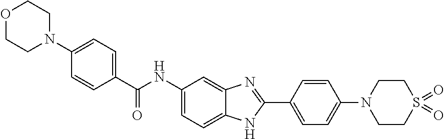 [SMILES: O=C(Nc1ccc2[nH]c(-c3ccc(N4CCS(=O)(=O)CC4)cc3)nc2c1)c1ccc(N2CCOCC2)cc1]

Compound 421 was prepared according to the procedure similar to that described in Scheme III from 3,4-dinitro-N-(4-morpholinobenzoyl)aniline and 4-(1,1-dioxo-4-thiomorpholino)benzaldehyde. [M+H]⁺ calcd for C₂₈H₂₉N₅O₄S: 532.19. Found: 532.02.

### Example 322

### N-(2-(4-((4-methylpiperazin-1-yl)methyl)phenyl)-1H-benzo[d]imidazol-5-yl)-4-morpholinobenzamide (Compound 422)

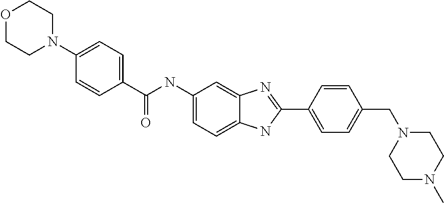 [SMILES: CN1CCN(Cc2ccc(-c3nc4cc(NC(=O)c5ccc(N6CCOCC6)cc5)ccc4[nH]3)cc2)CC1]

Compound 422 was prepared according to the procedure similar to that described in Scheme III from 3,4-dinitro-N-(4-morpholinobenzoyl)aniline and 4-piperazinomethylbenzaldehyde. [M+H]⁺ calcd for C₃₀H₃₄N₆O₂: 511.27. Found: 511.07.

### Example 323

### N-(2-(4-(4-Methylpiperazino)phenyl)-1H-benzimidazol-5-yl)-4-morpholinobenzamide (Compound 423)

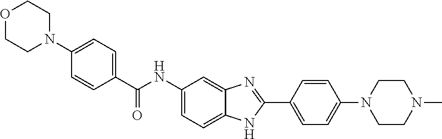 [SMILES: CN1CCN(c2ccc(-c3nc4cc(NC(=O)c5ccc(N6CCOCC6)cc5)ccc4[nH]3)cc2)CC1]

Compound 423 was prepared according to the procedure similar to that described in Scheme III from 3,4-dinitro-N-(4-morpholinobenzoyl)aniline and 4-(4-methylpiperazino)benzaldehyde. [M+H]⁺ calcd for C₂₉H₃₂N₆O₂: 497.26. Found: 496.98.

### Example 324

### N-(2-(2-Methoxy-4-morpholinophenyl)-1H-benzimidazol-5-yl)-4-morpholinobenzamide (Compound 424)

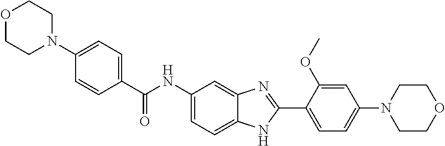 [SMILES: COc1cc(N2CCOCC2)ccc1-c1nc2cc(NC(=O)c3ccc(N4CCOCC4)cc3)ccc2[nH]1]

Compound 424 was prepared according to the procedure similar to that described in Scheme III from 3,4-dinitro-N-(4-morpholinobenzoyl)aniline and 2-methoxy-4-morpholinobenzaldehyde. [M+H]⁺ calcd for C₂₉H₃₁N₅O₄: 514.25. Found: 514.05.

### Example 325

### N-(2-(4-Morpholinophenyl)-1H-benzimidazol-5-yl)-3,5-dihydroxybenzamide (Compound 425)

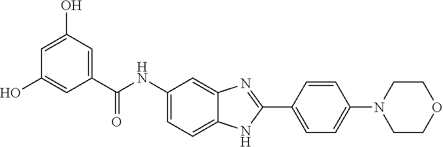 [SMILES: O=C(Nc1ccc2[nH]c(-c3ccc(N4CCOCC4)cc3)nc2c1)c1cc(O)cc(O)c1]

Compound 425 was prepared according to the procedure similar to that described in Scheme III from 3,4-dinitro-N-(3,5-dihydroxybenzoyl)aniline and 4-morpholinobenzaldehyde. [M+H]⁺ calcd for C₂₄H₂₂N₄O₄: 431.17. Found: 431.18.

### Example 326

### N-(2-(4-Morpholinophenyl)-1H-benzimidazol-5-yl)-4-hydroxybenzamide (Compound 426)

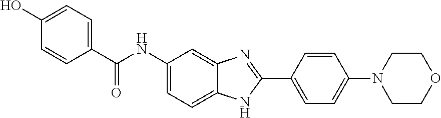 [SMILES: O=C(Nc1ccc2[nH]c(-c3ccc(N4CCOCC4)cc3)nc2c1)c1ccc(O)cc1]

Compound 426 was prepared according to the procedure similar to that described in Scheme III from 3,4-dinitro-N-(4-hydroxybenzoyl)aniline and 4-morpholinobenzaldehyde. [M+H]⁺ calcd for C₂₄H₂₂N₄O₃: 415.18. Found: 415.17.

### Example 327

### N-(2-(4-Morpholinophenyl)-1H-benzimidazol-5-yl)-2-hydroxybenzamide (Compound 427)

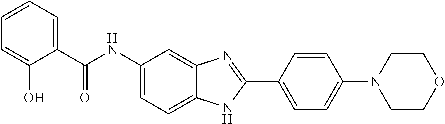 [SMILES: O=C(Nc1ccc2[nH]c(-c3ccc(N4CCOCC4)cc3)nc2c1)c1ccccc1O]

Compound 427 was prepared according to the procedure similar to that described in Scheme III from 3,4-dinitro-N-(2-hydroxybenzoyl)aniline and 4-morpholinobenzaldehyde. [M+H]⁺ calcd for C₂₄H₂₂N₄O₃: 415.18. Found: 414.98.

### Example 328

### 4-(4-(5-(5-(4-Morpholinophenyl)-1,3,4-oxadiazol-2-yl)-1H-benzimidazol-2-yl)phenyl)morpholine (Compound 428)

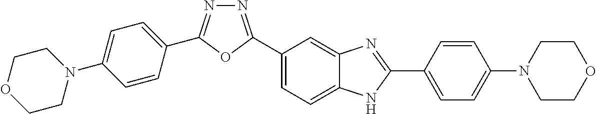 [SMILES: c1cc(N2CCOCC2)ccc1-c1nc2cc(-c3nnc(-c4ccc(N5CCOCC5)cc4)o3)ccc2[nH]1]

Compound 428 was prepared according to the procedure similar to that described in Scheme III from 3,4-dinitro-1-(5-(4-morpholinophenyl)-1,3,4-oxadiazol-2-yl)benzene and 4-morpholinobenzaldehyde. [M+H]⁺ calcd for C₂₉H₂₈N₆O₃: 590.23. Found: 508.98.

### Example 329

### 4-Dimethylamino-N-(2-(3-methoxyphenylamino)-1H-benzimidazol-5-yl)benzamide (Compound 429)

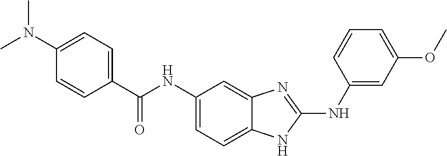

Compound 429 was prepared from 4-dimethylaminobenzoate and 5-amino-2-(3-methoxyphenylamino)benzimidazole by standard conditions. [M+H]⁺ calcd for C₂₃H₂₃N₅O₂: 402.19. Found: 401.96.

### Example 330

### N-(2-Dimethylamino)-1H-benzimidazol-5-yl)-2-(4-dimethylaminophenyl)-1H-benzimidazole-5-carbamide (Compound 430)

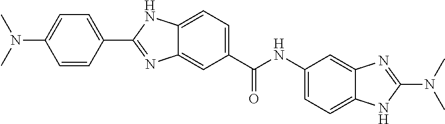

Compound 430 was prepared from 2-(4-dimethylaminophenyl)benzimidazole-5-carboxylate and 5-amino-2-(dimethylamino)benzimidazole by standard conditions. [M+H]⁺ calcd for C₂₅H₂₅N₇O: 440.21. Found: 440.01.

### Example 331

### 2-(4-(2-Hydroxyethoxy)phenyl)-N-2-(4-(2-hydroxyethoxy)phenyl)-1H-benzimidazol-5-yl)-1H-benzimidazole-6-carboxamide (Compound 431)

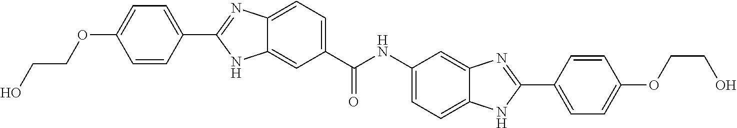 [SMILES: O=C(Nc1ccc2[nH]c(-c3ccc(OCCO)cc3)nc2c1)c1ccc2nc(-c3ccc(OCCO)cc3)[nH]c2c1]

Compound 431 was prepared according to the procedure similar to that described in Scheme V from 2-(4-(2-hydroxyethoxy)phenyl)-5-aminobenzimidazole and 2-(4-(2-hydroxyethoxy)phenyl)benzimidazole-5-carboxylate. [M+H]⁺ calcd for C₃₁H₂₇N₅O₅: 550.20. Found: 549.96.

### Example 332

### 2-(4-Cyanophenyl)-N-2-(4-(2-hydroxyethoxy)phenyl)-1H-benzimidazol-5-yl)-1H-benzimidazole-6-carboxamide (Compound 432)

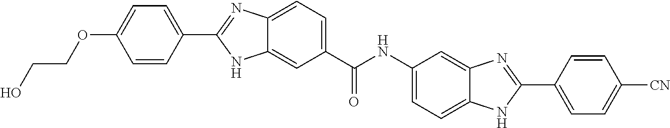 [SMILES: N#Cc1ccc(-c2nc3cc(NC(=O)c4ccc5nc(-c6ccc(OCCO)cc6)[nH]c5c4)ccc3[nH]2)cc1]

Compound 432 was prepared according to the procedure similar to that described in Scheme V from 2-(4-cyanophenyl)-5-aminobenzimidazole and 2-(4-(2-hydroxyethoxy)phenyl)benzimidazole-5-carboxylate. [M+H]⁺ calcd for C₃₀H₂₂N₆O₃: 515.18. Found: 514.92.

### Example 333

### 2-(4-bis(2-Hydroxyethyl)aminophenyl)-N-2-(4-(2-hydroxyethoxy)phenyl)-1H-benzimidazol-5-yl)-1H-benzimidazole-6-carboxamide (Compound 433)

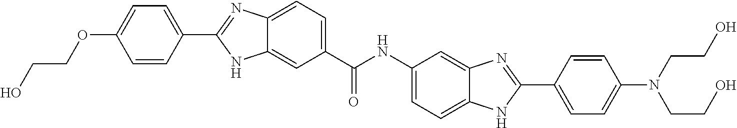 [SMILES: O=C(Nc1ccc2[nH]c(-c3ccc(N(CCO)CCO)cc3)nc2c1)c1ccc2nc(-c3ccc(OCCO)cc3)[nH]c2c1]

Compound 433 was prepared according to the procedure similar to that described in Scheme V from 2-(4-bis(2-hydroxyethyl)aminophenyl)-5-aminobenzimidazole and 2-(4-(2-hydroxyethoxy)phenyl)benzimidazole-5-carboxylate. [M+H]⁺ calcd for C₃₃H₃₂N₆O₅: 593.24. Found: 593.03.

### Example 334

### N,N′-bis-(2-(4-Morpholinophenyl)-1H-benzimidazol-5-yl)oxalamide (Compound 434)

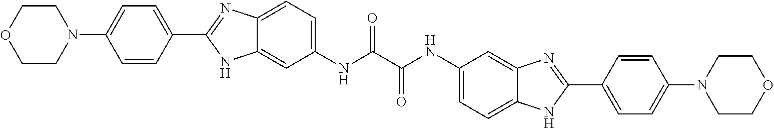 [SMILES: O=C(Nc1ccc2[nH]c(-c3ccc(N4CCOCC4)cc3)nc2c1)C(=O)Nc1ccc2nc(-c3ccc(N4CCOCC4)cc3)[nH]c2c1]

Compound 434 was prepared according to the procedure similar to that described in Scheme V from 2-(4-morpholinophenyl)-5-aminobenzimidazole and oxalic acid. [M+H]⁺ calcd for C₃₆H₃₄N₈O₄: 643.27. Found: 643.09.

### Example 335

### N,N′-bis(2-Dimethylamino-1H-benzimidazol-5-yl)terephthalamide (Compound 435)

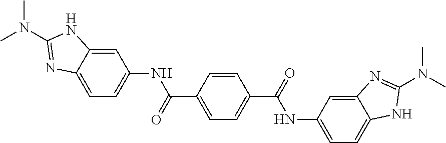

Compound 435 was prepared according to the procedure similar to that described in Scheme V from 2-dimethylamino-5-aminobenzimidazole and terephthalic acid. [M+H]⁺ calcd for C₂₆H₂₆N₈O₂: 483.22. Found: 483.02.

### Example 336

### N,N′-bis(2-Dimethylamino-1H-benzimidazol-5-yl)-3(E),3′(E)-(1,4-phenylene)bis(acrylamide) (Compound 436)

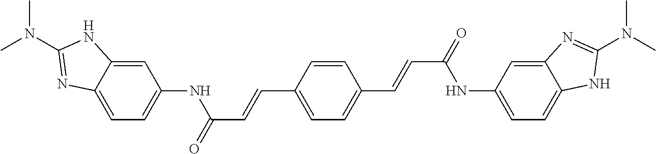

Compound 436 was prepared according to the procedure similar to that described in Scheme V from 2-dimethylamino-5-aminobenzimidazole and 3(E),3′(E)-(1,4-phenylene)bisacrylic acid. [M+H]⁺ calcd for C₃₀H₃₀N₈O₂: 535.25. Found: 535.09.

### Example 337

### N-(2-Dimethylamino)-1H-benzimidazol-5-yl)-4-(4-dimethylaminobenzamido)benzamide (Compound 437)

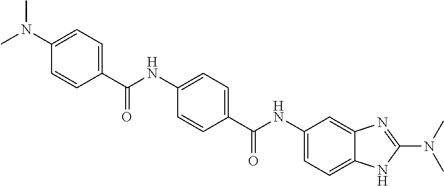

Compound 437 was prepared from 4-(4-dimethylaminobenzamido)benzoate and 5-amino-2-(dimethylamino)benzimidazole by standard conditions. [M+H]⁺ calcd for C₂₅H₂₆N₆O₂: 443.21. Found: 443.05.

### Example 338

### N-(2-(3-Methoxyphenyl)amino)-1H-benzimidazol-5-yl)-4-(4-dimethylaminobenzamido)benzamide (Compound 438)

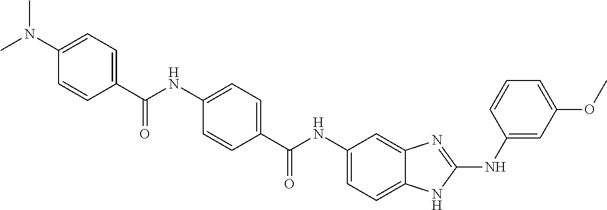

Compound 438 was prepared from 4-(4-dimethylaminobenzamido)benzoate and 5-amino-2-(3-methoxyphenyl)aminobenzimidazole by standard conditions. [M+H]⁺ calcd for C₃₀H₂₈N₆O₃: 521.22. Found: 521.06.

### Example 339

### N-(2-(3-Methoxyphenyl)amino-1H-benzimidazol-5-yl)-2-(4-dimethylaminophenyl-1H-benzimidazole-5-carbamide (Compound 439)

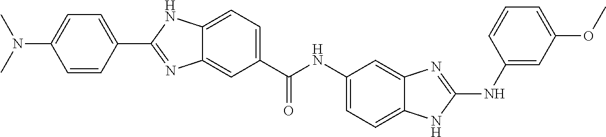

Compound 439 was prepared from 2-(4-dimethylaminophenyl)benzimidazole-5-carboxylate and 5-amino-2-(3-methoxyphenyl)aminobenzimidazole by standard conditions. [M+H]⁺ calcd for C₃₀H₂₇N₇O₂: 518.22. Found: 518.03.

### Example 340

### N-(2-(2,3-Dihydroxypropyl)amino-1H-benzimidazol-5-yl)-4-(4-dimethylaminobenzamido)benzamide (Compound 440)

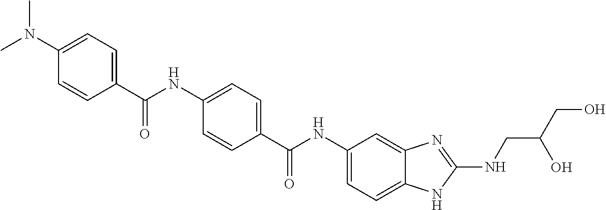 [SMILES: CN(C)c1ccc(C(=O)Nc2ccc(C(=O)Nc3ccc4[nH]c(NCC(O)CO)nc4c3)cc2)cc1]

Compound 440 was prepared from 4-(4-dimethylaminobenzamido)benzoate and 5-amino-2-(2,3-dihydroxypropylamino)benzimidazole by standard conditions. [M+H]⁺ calcd for C₂₆H₂₈N₆O₄: 489.22. Found: 489.01.

### Example 341

### N,N′-(1,4-Cyclohexane)bis(2-(4-dimethylaminophenyl)-1H-benzimidazole-5-carboxamide) (Compound 441)

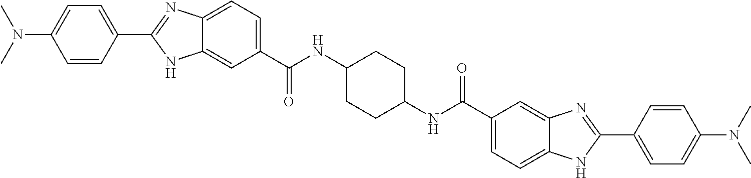

Compound 441 was prepared according to the procedure similar to that described in Scheme V from 1,4-cyclohexanediamine and 2-(4-dimethylaminophenyl)benzimidazole-5-carboxylate. [M+H]⁺ calcd for C₃₈H₄₀N₈O₂: 641.33. Found: 641.24.

### Example 342

### N-(2-(4-Dimethylaminophenyl)-1H-benzimidazol-5-yl)-4-(4-dimethylaminobenzamido)benzamide (Compound 442)

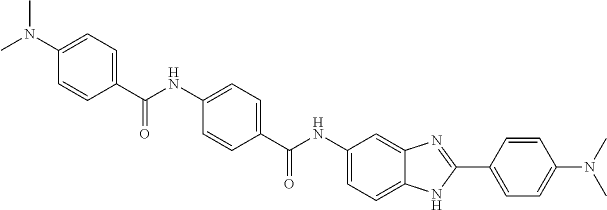

Compound 442 was prepared from 4-(4-dimethylaminobenzamido)benzoate and 5-amino-2-(4-dimethylaminophenyl)benzimidazole by standard conditions. [M+H]⁺ calcd for C₃₁H₃₀N₆O₂: 519.24. Found: 518.99.

### Example 343

### N-(4-(4-Dimethylaminobenzamido)phenyl)-2-(4-dimethylaminophenyl)-1H-benzimidazole-5-carboxamide (Compound 443)

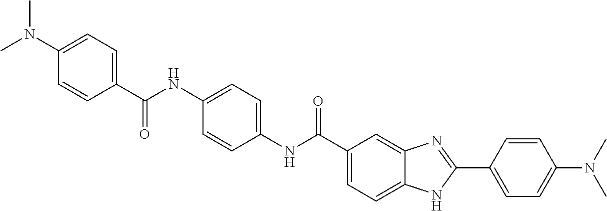

Compound 443 was prepared from 4-(4-dimethylaminobenzamido) aniline and 2-(4-dimethylaminophenyl)benzimidazole-5-carboxylate by standard conditions. [M+H]⁺ calcd for C₃₁H₃₀N₆O₂: 519.24. Found: 519.04.

### Example 344

### 4-(dimethylamino)-N-(4-((2-(4-methoxyphenyl)-1H-benzo[d]imidazol-5-yl)carbamoyl)phenyl)benzamide (Compound 444)

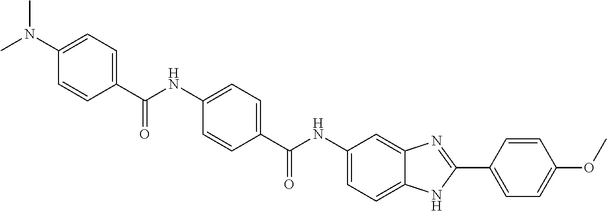

Compound 444 was prepared from 4-(4-dimethylaminobenzamido)aniline and 2-(4-methoxyphenyl)benzimidazole-5-carboxylate by standard conditions. [M+H]⁺ calcd for C₃₀H₂₇N₅O₃: 506.21. Found: 507.02.

### Example 345

### N-(4-(4-Dimethylaminobenzamido)phenyl)-1H-benzimidazole-5-carboxamide (Compound 445)

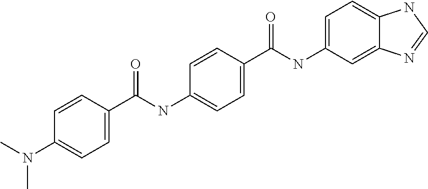 [SMILES: CN(C)c1ccc(C(=O)Nc2ccc(C(=O)Nc3ccc4[nH]cnc4c3)cc2)cc1]

Compound 445 was prepared from 4-(4-dimethylaminobenzamido) aniline and benzimidazole-5-carboxylate by standard conditions. [M+H]⁺ calcd for C₂₃H₂₁N₅O₂: 400.17. Found: 400.01.

### Example 346

### N-(2-(4-(4-(2-Morpholinoethyl)piperidino)phenyl)-1H-benzimidazol-5-yl)-4-morpholinobenzamide (Compound 446)

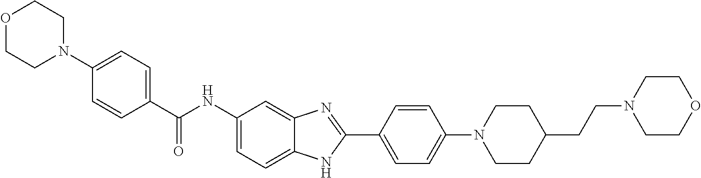 [SMILES: O=C(Nc1ccc2[nH]c(-c3ccc(N4CCC(CCN5CCOCC5)CC4)cc3)nc2c1)c1ccc(N2CCOCC2)cc1]

Compound 446 was prepared from 4-morpholinobenzoate and 5-amino-2-(4-(4-(2-morpholinoethyl)piperidino)phenyl)benzimidazole by standard conditions. [M+H]⁺ calcd for: C₃₅H₄₂N₆O₃; 595.33. Found: 595.12.

### Example 347

### N-(2-(4-(4-Morpholinocarbonyl)piperidino)phenyl)-1H-benzimidazol-5-yl)-4-morpholinobenzamide (Compound 447)

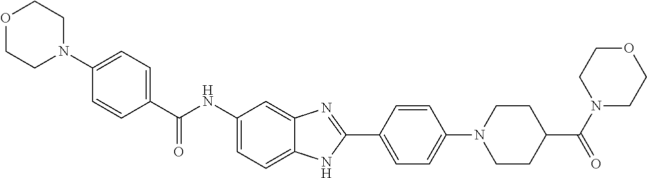 [SMILES: O=C(Nc1ccc2[nH]c(-c3ccc(N4CCC(C(=O)N5CCOCC5)CC4)cc3)nc2c1)c1ccc(N2CCOCC2)cc1]

Compound 447 was prepared from 4-morpholinobenzoate and 5-amino-2-(4-(4-morpholinocarbonyl)piperidino)phenylbenzimidazole by standard conditions. [M+H]⁺ calcd for C₃₄H₃₈N₆O₄: 595.30. Found: 595.13.

### Example 348

### N-(2-(4-(4-Morpholinocarbonylmethyl)piperazino)phenyl)-1H-benzimidazol-5-yl)-4-morpholinobenzamide (Compound 448)

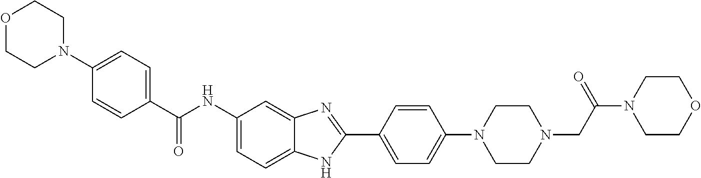 [SMILES: O=C(Nc1ccc2[nH]c(-c3ccc(N4CCN(CC(=O)N5CCOCC5)CC4)cc3)nc2c1)c1ccc(N2CCOCC2)cc1]

Compound 448 was prepared from 4-morpholinobenzoate and 5-amino-2-(4-(4-morpholinocarbonylmethyl)piperazino)phenylbenzimidazole by standard conditions. [M+H]⁺ calcd for C₃₄H₃₉N₇O₄: 610.31. Found: 610.11.

### Example 349

### 4-(5-Methylsulfonylamido)-1H-benzimidazol-2-yl)-N-(4-morpholinophenyl)benzamide (Compound 449)

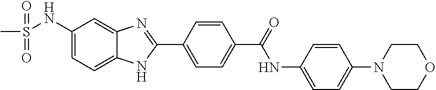 [SMILES: CS(=O)(=O)Nc1ccc2[nH]c(-c3ccc(C(=O)Nc4ccc(N5CCOCC5)cc4)cc3)nc2c1]

Compound 449 was prepared from 4-morpholinoaniline and 4-(5-methylsulfonylamino-1H-benzimidazol-2-yl)benzoate by standard conditions. [M+H]⁺ calcd for C₂₅H₂₅N₅O₄S: 492.17. Found: 491.97.

### Example 350

### 4-(5-Cyano-1H-benzimidazol-2-yl)-N-(4-morpholinophenyl)benzamide (Compound 450)

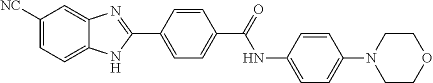 [SMILES: N#Cc1ccc2[nH]c(-c3ccc(C(=O)Nc4ccc(N5CCOCC5)cc4)cc3)nc2c1]

Compound 450 was prepared from 4-morpholinoaniline and 4-(5-cyano-1H-benzimidazol-2-yl)benzoate by standard conditions. [M+H]⁺ calcd for C₂₅H₂₁N₅O₂: 424.18. Found: 423.95.

### Example 351

### 4-(5-(3-Methyl-5-oxo-2,5-dihydro-1H-pyrazol-1-yl)-1H-benzimidazol-2-yl)-N-(4-morpholinophenyl)benzamide (Compound 451)

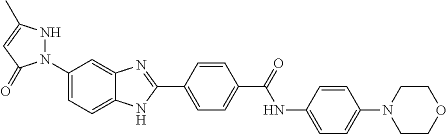 [SMILES: Cc1cc(=O)n(-c2ccc3[nH]c(-c4ccc(C(=O)Nc5ccc(N6CCOCC6)cc5)cc4)nc3c2)[nH]1]

Compound 451 was prepared from 4-morpholinoaniline and 4-(5-(3-methyl-5-oxo-2,5-dihydro-1H-pyrazol-1-yl)-1H-benzimidazol-2-yl)benzoate by standard conditions. [M+H]⁺ calcd for C₂₈H₂₆N₆O₃: 495.22. Found: 495.01.

### Example 352

### 5-Amino-(2-(4-(4-morpholinocarbonylmethyl)piperazino)phenyl)-1H-benzimidazole (Compound 452)

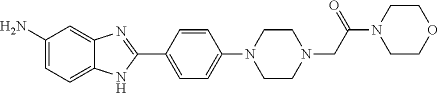 [SMILES: Nc1ccc2[nH]c(-c3ccc(N4CCN(CC(=O)N5CCOCC5)CC4)cc3)nc2c1]

Compound 452 was prepared according to the procedure similar to that described in Scheme III from 3,4-dinitroaniline and 2-(4-(4-morpholinocarbonylmethyl)piperazino)benzaldehyde. [M+H]⁺ calcd for C₂₃H₂₈N₆O₂: 421.23. Found: 420.98.

### Example 353

### 4-(5-Amino-1H-benzimidazol-2-yl)-N-(4-morpholinophenyl)benzamide (Compound 453)

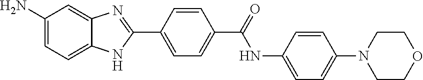 [SMILES: Nc1ccc2[nH]c(-c3ccc(C(=O)Nc4ccc(N5CCOCC5)cc4)cc3)nc2c1]

Compound 453 was prepared from 4-morpholinoaniline and 4-(5-amino-1H-benzimidazol-2-yl)benzoate by standard conditions. [M+H]⁺ calcd for C₂₄H₂₃N₅O₂: 414.19. Found: 413.97.

### Example 354

### 4-(5-Acetamino-1H-benzimidazol-2-yl)-N-(4-morpholinophenyl)benzamide (Compound 454)

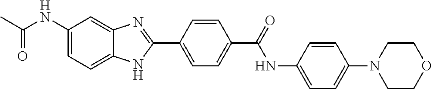 [SMILES: CC(=O)Nc1ccc2[nH]c(-c3ccc(C(=O)Nc4ccc(N5CCOCC5)cc4)cc3)nc2c1]

Compound 454 was prepared from 4-morpholinoaniline and 4-(5-acetamino-1H-benzimidazol-2-yl)benzoate by standard conditions. [M+H]⁺ calcd for C₂₆H₂₅N₅O₃: 456.20. Found: 455.95.

### Example 355

### 4-(5-(2,2,2-Trifluoroethyl)amino-1H-benzimidazol-2-yl)-N-(4-morpholinophenyl)benzamide (Compound 455)

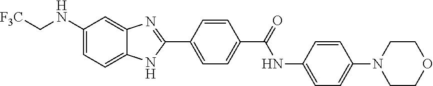 [SMILES: O=C(Nc1ccc(N2CCOCC2)cc1)c1ccc(-c2nc3cc(NCC(F)(F)F)ccc3[nH]2)cc1]

Compound 455 was prepared from 4-morpholinoaniline and 4-(5-bis(2,2,2-trifluoroethyl)amino-1H-benzimidazol-2-yl)benzoate by standard conditions. [M+H]⁺ calcd for C₂₆H₂₄F₃N₅O₂: 496.19. Found: 496.02.

### Example 356

### 4-(5-bis(2,2,2-Trifluoroethyl)amino-1H-benzimidazol-2-yl)-N-(4-morpholinophenyl)benzamide (Compound 456)

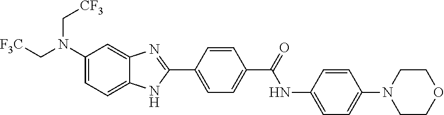 [SMILES: O=C(Nc1ccc(N2CCOCC2)cc1)c1ccc(-c2nc3cc(N(CC(F)(F)F)CC(F)(F)F)ccc3[nH]2)cc1]

Compound 456 was prepared from 4-morpholinoaniline and 4-(5-bis(2,2,2-trifluoroethyl)amino-1H-benzimidazol-2-yl)benzoate by standard conditions. [M+H]⁺ calcd for C₂₈H₂₅F6N₅O₂: 578.19. Found: 578.04.

### Example 357

### 4-(5-Morpholino-1H-benzimidazol-2-yl)-N-(4-morpholinophenyl)benzamide (Compound 457)

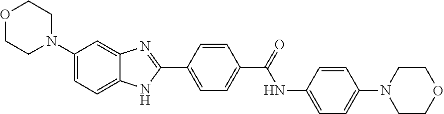 [SMILES: O=C(Nc1ccc(N2CCOCC2)cc1)c1ccc(-c2nc3cc(N4CCOCC4)ccc3[nH]2)cc1]

Compound 457 was prepared from 4-morpholinoaniline and 4-(5-morpholino-1H-benzimidazol-2-yl)benzoate by standard conditions. [M+H]⁺ calcd for C₂₈H₂₉N₅O₃: 484.23. Found: 484.01.

### Example 358

### 4-(1H-Benzimidazol-2-yl)-N-(4-morpholinophenyl)benzamide (Compound 458)

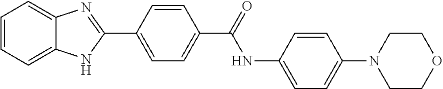 [SMILES: O=C(Nc1ccc(N2CCOCC2)cc1)c1ccc(-c2nc3ccccc3[nH]2)cc1]

Compound 458 was prepared from 4-morpholinoaniline and 4-(1H-benzimidazol-2-yl)benzoate by standard conditions. [M+H]⁺ calcd for C₂₄H₂₂N₄O₂: 399.17. Found: 398.98.

### Example 359

### 4-(5-Hydroxy-1H-benzimidazol-2-yl)-N-(4-morpholinophenyl)benzamide (Compound 459)

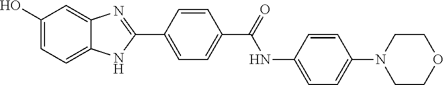 [SMILES: O=C(Nc1ccc(N2CCOCC2)cc1)c1ccc(-c2nc3cc(O)ccc3[nH]2)cc1]

Compound 459 was prepared from 4-morpholinoaniline and 4-(5-hydroxy-1H-benzimidazol-2-yl)benzoate by standard conditions. [M+H]⁺ calcd for C₂₄H₂₂N₄O₃: 415.17. Found: 415.05.

### Example 360

### 4-(5-Methoxy-1H-benzimidazol-2-yl)-N-(4-morpholinophenyl)benzamide (Compound 460)

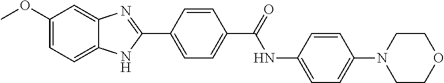 [SMILES: COc1ccc2[nH]c(-c3ccc(C(=O)Nc4ccc(N5CCOCC5)cc4)cc3)nc2c1]

Compound 460 was prepared from 4-morpholinoaniline and 4-(5-methoxy-1H-benzimidazol-2-yl)benzoate by standard conditions. [M+H]⁺ calcd for C₂₅H₂₄N₄O₃: 429.18. Found: 428.97.

### Example 361

### 4-(1H-Benzimidazol-2-yl)-N-(4-(4-cyclopropanecarbonylpiperazino)phenyl)benzamide (Compound 461)

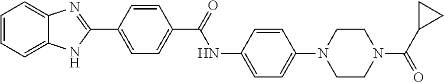 [SMILES: O=C(Nc1ccc(N2CCN(C(=O)C3CC3)CC2)cc1)c1ccc(-c2nc3ccccc3[nH]2)cc1]

Compound 461 was prepared from 4-(4-cyclopropanecarbonyl)piperazinoaniline and 4-(1H-benzimidazol-2-yl)benzoate by standard conditions. [M+H]⁺ calcd for C₂₈H₂₇N₅O₂: 466.22. Found: 465.99.

### Example 362

### 4-(5-Amino-1H-benzimidazol-2-yl)-N-(4-(4-cyclopropanecarbonylpiperazino)phenyl)benzamide (Compound 462)

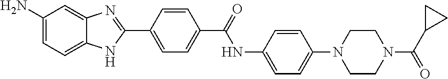 [SMILES: Nc1ccc2[nH]c(-c3ccc(C(=O)Nc4ccc(N5CCN(C(=O)C6CC6)CC5)cc4)cc3)nc2c1]

Compound 462 was prepared from 4-(4-cyclopropanecarbonyl)piperazinoaniline and 4-(5-amino-1H-benzimidazol-2-yl)benzoate by standard conditions. [M+H]⁺ calcd for C₂₈H₂₈N₆O₂: 481.23. Found: 481.05.

### Example 363

### 4-(5-(2,2,2-Trifluoroethoxy)-1H-benzimidazol-2-yl)-N-(4-morpholinophenyl)benzamide (Compound 463)

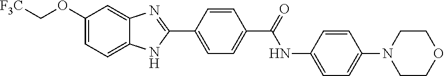 [SMILES: O=C(Nc1ccc(N2CCOCC2)cc1)c1ccc(-c2nc3cc(OCC(F)(F)F)ccc3[nH]2)cc1]

Compound 463 was prepared from 4-morpholinoaniline and 4-(5-(2,2,2-trifluoroethoxy)-1H-benzimidazol-2-yl)benzoate by standard conditions. [M+H]calcd for C₂₆H₂₃F₃N₄O₃: 497.17. Found: 496.97.

### Example 364

### N,N′-(4,4′-(1,3,4-Oxadiazole-2,5-diyl)bis(4,1-phenylene))bis(4-dimethylaminobenzamide) (Compound 464)

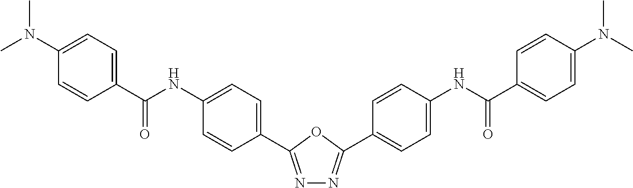

Compound 464 was prepared according to the procedure described in Scheme IV from 4,4′-(1,3,4-oxadiazole-2,5-diyl)bis(4,1-phenylene)diamine and 4-dimethylaminobenzoate. ¹H NMR (500 MHz, DMSO-d₆) δ 10.20 (s, 2H), 8.06 (dq, J=2, 9 Hz, 8H), 7.90 (d, J=9 Hz, 4H), 6.77 (d, J=9 Hz, 4H), 3.01 (s, 12H).

### Example 365

### N,N′-bis(2-(4-Morpholinophenylamino)-2-oxoethyl)biphenyl-4,4′-dicarboxamide (Compound 465)

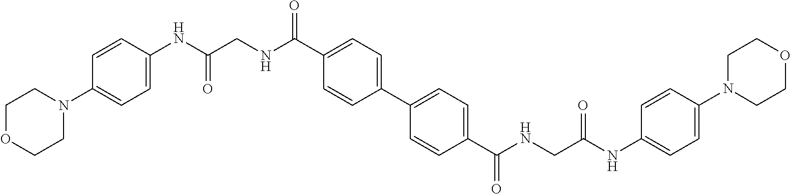 [SMILES: O=C(CNC(=O)c1ccc(-c2ccc(C(=O)NCC(=O)Nc3ccc(N4CCOCC4)cc3)cc2)cc1)Nc1ccc(N2CCOCC2)cc1]

Compound 465 was prepared according to the procedure described in Scheme IV from 4-morpholinoaniline, glycine, and 4,4′-biphenyldicarboxylate. [M+H]⁺ calcd for C₃₈H₄₀N₆O₆: 677.30. Found: 677.23.

### Example 366

### 4′-(2-(4-Morpholinophenylamino)-2-oxoethylcarbamoyl)biphenyl-4-carboxamide (Compound 466)

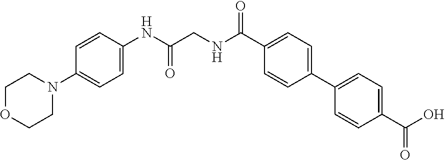 [SMILES: O=C(CNC(=O)c1ccc(-c2ccc(C(=O)O)cc2)cc1)Nc1ccc(N2CCOCC2)cc1]

Compound 466 was prepared according to the procedure described in Scheme IV from 4-morpholinoaniline, glycine, and 4,4′-biphenyldicarboxylic acid. [M+H]⁺ calcd for C₂₆H₂₅N₃O₅: 460.18. Found: 459.92.

### Example 367

### N-(2-(4-Morpholinophenyl)amino-2-oxoethyl)-4-dimethylaminobenzamide (Compound 467)

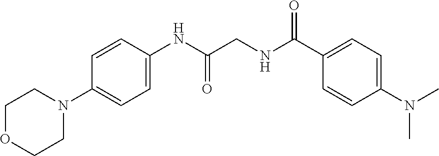 [SMILES: CN(C)c1ccc(C(=O)NCC(=O)Nc2ccc(N3CCOCC3)cc2)cc1]

Compound 467 was prepared according to the procedure described in Scheme IV from 4-morpholinoaniline, glycine, and 4-dimethylaminobenzoate. ¹H NMR (500 MHz, CDCl₃) δ 8.46 (s, 1H), 7.74 (s, J=9 Hz, 2H), 6.82 (t, J=5.5 Hz, 1H), 6.68 (d, J=9 Hz, 2H), 4.25 (d, J=5.5 Hz, 2H), 3.85 (m, 4H), 3.11 (m, 4H), 3.04 (s, 6H).

### Example 368

### N,N′-bis(5-(Furan-2-yl)-1H-pyrazol-3-yl)biphenyl-4,4′-dicarboxamide (Compound 468)

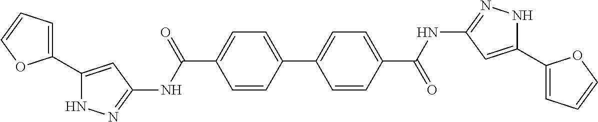 [SMILES: O=C(Nc1cc(-c2ccco2)[nH]n1)c1ccc(-c2ccc(C(=O)Nc3cc(-c4ccco4)[nH]n3)cc2)cc1]

Compound 468 was prepared from 3-amino-5-(2-furanyl)pyrazole and 4,4′-biphenyldicarboxylate. [M+H]⁺ calcd for C₂₈H₂₁N₆O₄: 505.16. Found: 504.93.

### Example 369

### N,N′-bis(5-(Thiophen-2-yl)-1H-pyrazol-3-yl)biphenyl-4,4′-dicarboxamide (Compound 469)

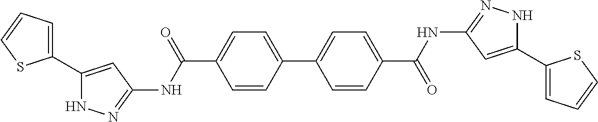 [SMILES: O=C(Nc1cc(-c2cccs2)[nH]n1)c1ccc(-c2ccc(C(=O)Nc3cc(-c4cccs4)[nH]n3)cc2)cc1]

Compound 469 was prepared from 3-amino-5-(2-thienyl)pyrazole and 4,4′-biphenyldicarboxylate. [M+H]⁺ calcd for C₂₈H₂₁N₆O₂S₂: 537.12. Found: 536.93.

### Example 370

### 1,1′-bis(1,4-Phenylene)bis(3-(5-(4-methoxyphenyl)-1H-pyrazol-3-yl)thiourea) (Compound 470)

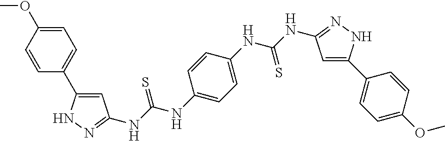

Compound 470 was prepared from 3-amino-5-(4-methoxyphenyl)pyrazole and 1,4-phenylenediamine. [M+H]⁺ calcd for C₂₈H₂₇N₈O₂S₂: 571.17. Found: 570.89.

### Example 371

### N-(5-(4-Methoxyphenyl)-1H-pyrazol-3-yl)-N′-(5-(4-methylphenyl)-1H-pyrazol-3-yl)terephthalamide (Compound 471)

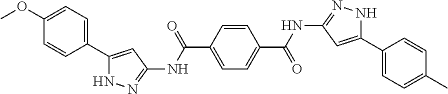

Compound 471 was prepared from 3-amino-5-(4-methoxyphenyl)pyrazole, 3-amino-5-(4-methylphenyl)pyrazole, and terephthalic acid. [M+H]⁺ calcd for C₂₈H₂₅N₆O₃: 493.20. Found: 492.92.

### Example 372

### N,N′-bis(5-(4-Methylphenyl)-1H-pyrazol-3-yl)terephthalamide (Compound 472)

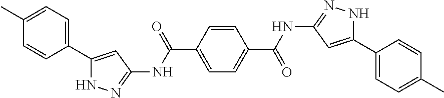

Compound 472 was prepared from 3-amino-5-(4-methylphenyl)pyrazole and terephthalic acid. [M+H]⁺ calcd for C₂₈H₂₅N₆O₂: 477.20. Found: 476.92.

### Example 373

### N,N′-bis(5-(4-Methoxyphenyl)-1H-pyrazol-3-yl)terephthalamide (Compound 473)

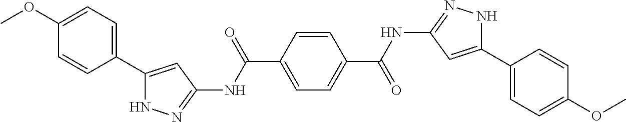

Compound 473 was prepared from 3-amino-5-(4-methoxyphenyl)pyrazole and terephthalic acid. [M+H]⁺ calcd for C₂₈H₂₅N₆O₄: 509.19. Found: 508.91.

### Example 374

### N,N′-bis(5-(4-Methoxyphenyl)-1H-pyrazol-3-yl)biphenyl-4,4′-dicarboxamide (Compound 474)

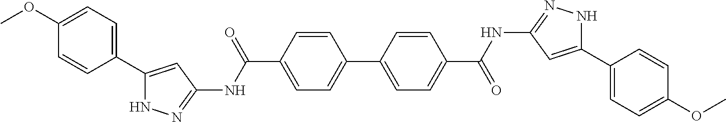

Compound 474 was prepared from 3-amino-5-(4-methoxyphenyl)pyrazole and biphenyl-4,4′-dicarboxylic acid. [M+H]⁺ calcd for C₃₄H₂₉N₆O₄: 585.23. Found: 585.01.

### Example 375

### N,N′-bis(5-Phenyl-1H-pyrazol-3-yl)biphenyl-4,4′-dicarboxamide (Compound 475)

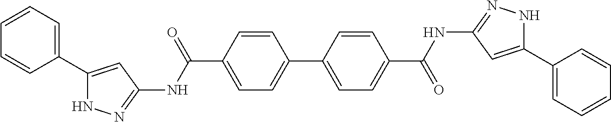 [SMILES: O=C(Nc1cc(-c2ccccc2)[nH]n1)c1ccc(-c2ccc(C(=O)Nc3cc(-c4ccccc4)[nH]n3)cc2)cc1]

Compound 475 was prepared from 3-amino-5-phenylpyrazole and biphenyl-4,4′-dicarboxylic acid. [M+H]⁺ calcd for C₃₂H₂₅N₆O₂: 525.20. Found: 524.98.

### Example 376

### N,N′-bis(5-(4-Methylphenyl)-1H-pyrazol-3-yl)biphenyl-4,4′-dicarboxamide (Compound 476)

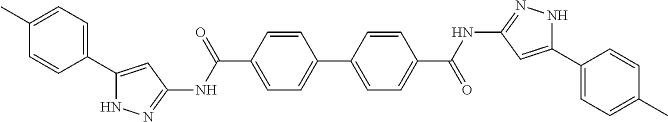

Compound 476 was prepared from 3-amino-5-(4-methylphenyl)pyrazole and biphenyl-4,4′-dicarboxylic acid. [M+H]⁺ calcd for C₃₄H₂₉N₆O₂: 553.24. Found: 553.06.

### Example 377

### N,N′-bis(5-(4-Methoxyphenyl)-1H-pyrazol-3-yl)-3(E),3′(E)-(1,4-phenylene)bis(acrylamide) (Compound 477)

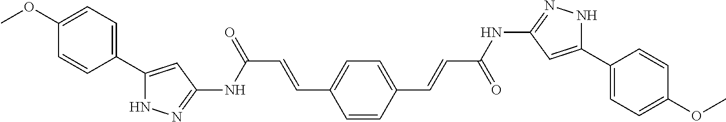

Compound 477 was prepared from 3-amino-5-(4-methoxyphenyl)pyrazole and 3(E),3′(E)-(1,4-phenylene)bis(acrylic acid). [M+H]⁺ calcd for C₃₂H₂₈N₆O₄: 561.23. Found: 561.05.

### Example 378

### 4,4′-(1,3-Phenylenebis(methylene)bis(azanediyl)bis(methylidene)bis(3-methyl-1-phenyl-1H-pyrazol-5(4H)-one) (Compound 478)

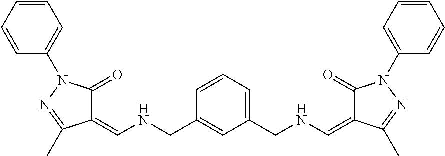 [SMILES: CC1=NN(c2ccccc2)C(=O)/C1=C\NCc1cccc(CN/C=C2\C(=O)N(c3ccccc3)N=C2C)c1]

Compound 478 was prepared from 3-methyl-1-phenyl-1H-pyrazol-5(4H)-one and 1,3-phenylenebis(methylamine). [M+H]⁺ calcd for C₃₀H₂₈N₆O₂: 505.23. Found: 505.07.

### Example 379

### N-(2-(4-(Pyridin-2-yl)phenyl)-1H-benzimidazol-5-yl)benzamide (Compound 479)

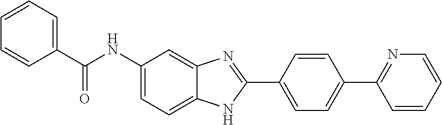 [SMILES: O=C(Nc1ccc2[nH]c(-c3ccc(-c4ccccn4)cc3)nc2c1)c1ccccc1]

Compound 479 was prepared from 5-amino-2-(4-(pyridin-2-yl)phenyl)-1H-benzimidazole and benzoate by standard conditions. [M+H]⁺ calcd for C₂₅H₁₈N₄O: 391.16. Found: 390.91.

### Example 380

### 4-(5-Cyclopropylpropiolamido-1H-benzimidazol-2-yl)-N-(4-morpholinophenyl)benzamide (Compound 480)

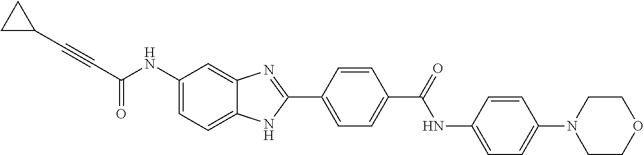 [SMILES: O=C(C#CC1CC1)Nc1ccc2[nH]c(-c3ccc(C(=O)Nc4ccc(N5CCOCC5)cc4)cc3)nc2c1]

Compound 480 was prepared from 4-morpholinoaniline and 4-(5-amino-1H-benzimidazol-2-yl)benzoate by standard conditions. [M+H]⁺ calcd for C₃₀H₂₇N₅O₃: 506.22. Found: 506.01.

### Example 381

### 4-(5-Trifluoromethoxy-1H-benzimidazol-2-yl)-N-(4-morpholinophenyl)benzamide (Compound 481)

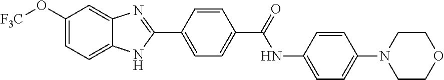 [SMILES: O=C(Nc1ccc(N2CCOCC2)cc1)c1ccc(-c2nc3cc(OC(F)(F)F)ccc3[nH]2)cc1]

Compound 481 was prepared from 4-morpholinoaniline and 4-(5-trifluoromethoxy-1H-benzimidazol-2-yl)benzoate by standard conditions. [M+H]⁺ calcd for C₂₅H₂₁F₃N₄O₃: 483.17. Found: 482.93.

### Example 382

### 4-(5-Aminosulfonyl-1H-benzimidazol-2-yl)-N-(4-morpholinophenyl)benzamide (Compound 482)

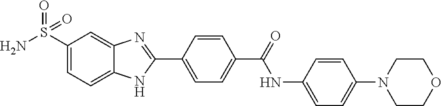 [SMILES: NS(=O)(=O)c1ccc2[nH]c(-c3ccc(C(=O)Nc4ccc(N5CCOCC5)cc4)cc3)nc2c1]

Compound 482 was prepared from 4-morpholinoaniline and 4-(5-aminosulfonyl-1H-benzimidazol-2-yl)benzoate by standard conditions. [M+H]⁺ calcd for C₂₄H₂₃N₅O₄S: 478.16. Found: 477.93.

### Example 383

### N-(2-(4-(4-Dimethylaminophenylcarbamoyl)piperidino)-1H-benzimidazol-5-yl)4-dimethylaminobenzamide (Compound 483)

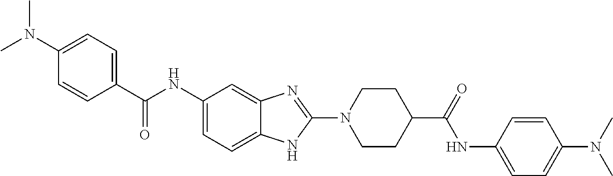

Compound 483 was prepared from 4-dimethylaminoaniline, 4-dimethylaminobenzoate, and 1-(5-amino-1H-benzimidazol-2-yl)piperidino-4-carboxylic acid by standard conditions. [M+H]⁺ calcd for C₃₀H₃₅N₇O₂: 526.29. Found: 526.13.

### Example 384

### N-(4-(4-Ethylaminobenzoyl)phenyl)-4-morpholinobenzamide (Compound 484)

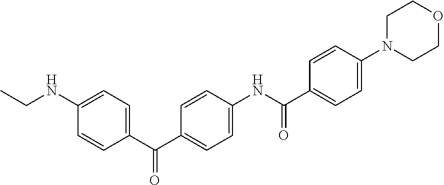 [SMILES: CCNc1ccc(C(=O)c2ccc(NC(=O)c3ccc(N4CCOCC4)cc3)cc2)cc1]

Compound 484 was prepared according to the procedure described in Scheme IV from 4,4′-diaminobenzophenone and 4-morpholinobenzoate. [M+H]⁺ calcd for C₂₆H₂₇N₃O₃: 430.03. Found: 430.03.

### Example 385

### N-(4-(4-Aminobenzoyl)phenyl)-4-morpholinobenzamide (Compound 485)

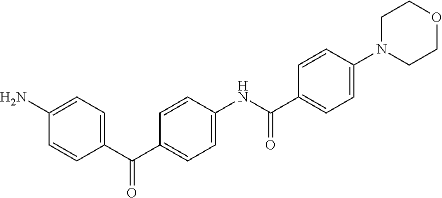 [SMILES: Nc1ccc(C(=O)c2ccc(NC(=O)c3ccc(N4CCOCC4)cc3)cc2)cc1]

Compound 485 was prepared according to the procedure described in Scheme IV from 4,4′-diaminobenzophenone and 4-morpholinobenzoate. [M+H]⁺ calcd for C₂₄H₂₃N₃O₃: 402.08. Found: 402.03.

### Example 386

### N-(4-(4-(2-Thienyl)carboxamido)benzoyl)phenyl)-4-fluorobenzamide (Compound 486)

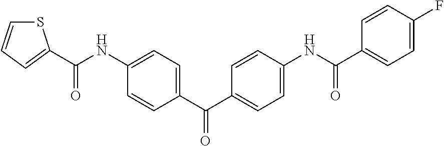 [SMILES: O=C(Nc1ccc(C(=O)c2ccc(NC(=O)c3cccs3)cc2)cc1)c1ccc(F)cc1]

Compound 486 was prepared according to the procedure described in Scheme IV from 4,4′-diaminobenzophenone and 4-fluorobenzoate. [M+H]⁺ calcd for C₂₅H₁₇FN₂O₃S: 445.00. Found: 444.94.

### Example 387

### N-(1-(4-Cyclopropanecarboxamidophenyl)-1H-indazol-5-yl)-4-(4-tert-butyloxycarbonylpiperazino)benzamide (Compound 487)

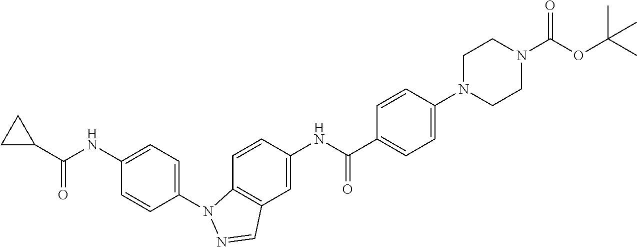 [SMILES: CC(C)(C)OC(=O)N1CCN(c2ccc(C(=O)Nc3ccc4c(cnn4-c4ccc(NC(=O)C5CC5)cc4)c3)cc2)CC1]

Compound 487 was prepared according to the procedure described in Scheme IV from 5-amino-1-(4-aminophenyl)indazole and 4-(4-tert-butyloxycarbonylpiperazino)benzoate. [M+H]⁺ calcd for C₃₃H₃₆N₆O₄: 581.28. Found: 581.15.

### Example 388

### N-(1-(4-Cyclopropanecarboxamidophenyl)-1H-indazol-5-yl)-4-(4-cyclopropanecarbonylpiperazino)benzamide (Compound 488)

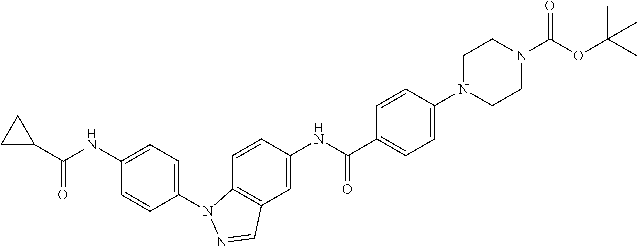 [SMILES: CC(C)(C)OC(=O)N1CCN(c2ccc(C(=O)Nc3ccc4c(cnn4-c4ccc(NC(=O)C5CC5)cc4)c3)cc2)CC1]

Compound 488 was prepared according to the procedure described in Scheme IV from 5-amino-1-(4-aminophenyl)indazole and 4-(4-cyclopropanecarbonylpiperazino)benzoate. [M+H]⁺ calcd for C₃₂H₃₂N₆O₃: 549.25. Found: 549.16.

### Example 389

### N-(4-(4-(2-Thienylcarboxamido)benzoyl)phenyl)-4-morpholinobenzamide (Compound 489)

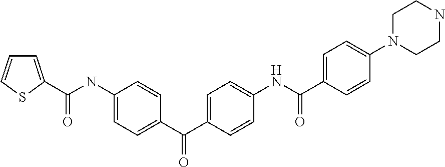 [SMILES: O=C(Nc1ccc(C(=O)c2ccc(NC(=O)c3cccs3)cc2)cc1)c1ccc(N2CCNCC2)cc1]

Compound 489 was prepared according to the procedure described in Scheme IV from 4,4′-diaminobenzophenone and 4-morpholinobenzoate. ¹H NMR (500 MHz, CD₃OD) δ 7.98 (d, J=5 Hz, 1H), 7.90 (dd, J=2.5, 10.6 Hz, 6H), 7.82 (m, 4H), 7.76 (d, J=5 Hz, 1H), 7.20 (t, J=5 Hz, 1H), 7.03 (d, J=10.6 Hz, 2H), 3.33 (m, 4H), 2.98 (m, 4H).

### Example 390

### N-(4-(4-(3-Thienylcarboxamido)benzoyl)phenyl)-4-morpholinobenzamide (Compound 490)

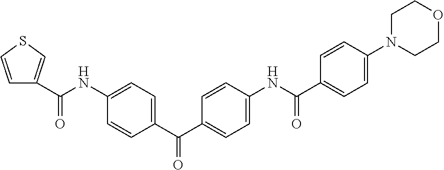 [SMILES: O=C(Nc1ccc(C(=O)c2ccc(NC(=O)c3ccsc3)cc2)cc1)c1ccc(N2CCOCC2)cc1]

Compound 490 was prepared according to the procedure described in Scheme IV from 4,4′-diaminobenzophenone and 4-morpholinobenzoate. [M+H]⁺ calcd for C₂₉H₂₅N₃O₄S: 512.11. Found: 512.09.

### Example 391

### N-(4-(4-(2-(5-Methylthienyl)carboxamido)benzoyl)phenyl)-4-morpholinobenzamide (Compound 491)

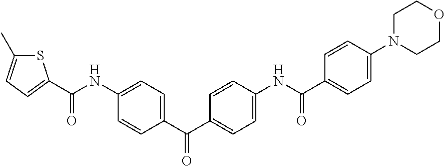 [SMILES: Cc1ccc(C(=O)Nc2ccc(C(=O)c3ccc(NC(=O)c4ccc(N5CCOCC5)cc4)cc3)cc2)s1]

Compound 491 was prepared according to the procedure described in Scheme IV from 4,4′-diaminobenzophenone and 4-morpholinobenzoate. [M+H]⁺ calcd for C₃₀H₂₇N₃O₄S: 526.14. Found: 526.13.

### Example 392

### N-(4-(4-(2-Thienyl)carboxamido)benzoyl)phenyl)-4-(2-hydroxyethoxy)benzamide (Compound 492)

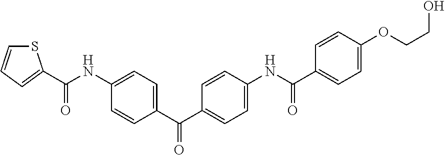 [SMILES: O=C(Nc1ccc(C(=O)c2ccc(NC(=O)c3cccs3)cc2)cc1)c1ccc(OCCO)cc1]

Compound 492 was prepared according to the procedure described in Scheme IV from 4,4′-diaminobenzophenone and 4-(2-hydroxyethoxy)benzoate. [M+H]⁺ calcd for C₂₇H₂₂N₂O₅S: 487.06. Found: 487.05.

### Example 393

### N-(4-(4-(2-Methoxyethylamino)benzoyl)phenyl)-4-morpholinobenzamide (Compound 493)

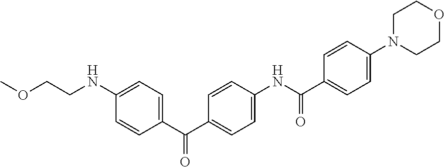 [SMILES: COCCNc1ccc(C(=O)c2ccc(NC(=O)c3ccc(N4CCOCC4)cc3)cc2)cc1]

Compound 493 was prepared according to the procedure described in Scheme IV from 4,4′-diaminobenzophenone and 4-morpholinobenzoate. [M+H]⁺ calcd for C₂₇H₂₉N₃O₄: 460.06. Found: 460.12.

### Example 394

### N-(4-(4-(2-(3-Methylthienyl)carboxamido)benzoyl)phenyl)-4-morpholinobenzamide (Compound 494)

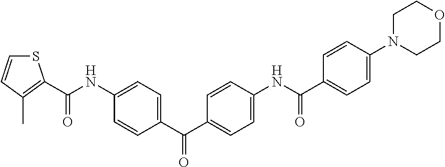 [SMILES: Cc1ccsc1C(=O)Nc1ccc(C(=O)c2ccc(NC(=O)c3ccc(N4CCOCC4)cc3)cc2)cc1]

Compound 494 was prepared according to the procedure described in Scheme IV from 4,4′-diaminobenzophenone and 4-morpholinobenzoate. [M+H]⁺ calcd for C₃₀H₂₇N₃O₄S: 526.14. Found: 526.13.

### Example 395

### 4-Morpholino-N-(4-(4-(thiophen-2-ylmethylamino)benzoyl)phenyl)benzamide (Compound 495)

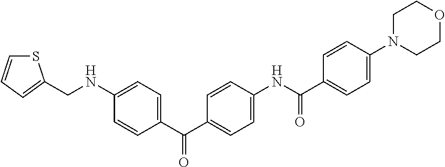 [SMILES: O=C(Nc1ccc(C(=O)c2ccc(NCc3cccs3)cc2)cc1)c1ccc(N2CCOCC2)cc1]

Compound 495 was prepared according to the procedure described in Scheme IV from 4,4′-diaminobenzophenone and 4-morpholinobenzoate. [M+H]⁺ calcd for C₂₉H₂₇N₃O₃S: 498.13. Found: 498.05.

### Example 396

### N-(4-(4-(2-(4-Methylthienyl)carboxamido)benzoyl)phenyl)-4-morpholinobenzamide (Compound 496)

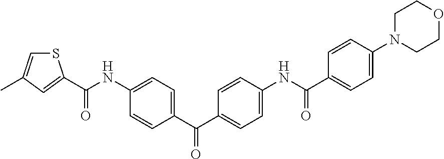 [SMILES: Cc1csc(C(=O)Nc2ccc(C(=O)c3ccc(NC(=O)c4ccc(N5CCOCC5)cc4)cc3)cc2)c1]

Compound 496 was prepared according to the procedure described in Scheme IV from 4,4′-diaminobenzophenone and 4-morpholinobenzoate. [M+H]⁺ calcd for C₃₀H₂₇N₃O₄S: 526.14. Found: 526.06.

### Example 397

### 4-Morpholino-N-(4-(4-(cyclopropylmethylamino)benzoyl)phenyl)benzamide (Compound 497)

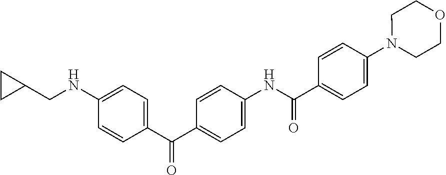 [SMILES: O=C(Nc1ccc(C(=O)c2ccc(NCC3CC3)cc2)cc1)c1ccc(N2CCOCC2)cc1]

Compound 497 was prepared according to the procedure described in Scheme IV from 4,4′-diaminobenzophenone and 4-morpholinobenzoate. [M+H]⁺ calcd for C₂₈H₂₉N₃O₃: 456.07. Found: 456.31.

### Example 398

### N-(4-(4-(4-Morpholinobenzamido)benzoyl)phenyl)pyrimidine-5-carboxamide (Compound 498)

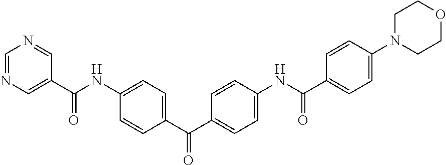 [SMILES: O=C(Nc1ccc(C(=O)c2ccc(NC(=O)c3cncnc3)cc2)cc1)c1ccc(N2CCOCC2)cc1]

Compound 498 was prepared according to the procedure described in Scheme IV from 4,4′-diaminobenzophenone and 4-morpholinobenzoate. [M+H]⁺ calcd for C₂₉H₂₅N₅O₄: 508.06. Found: 508.04.

### Example 399

### N-(4-(4-(4-Morpholinobenzamido)benzoyl)phenyl)-1H-pyrrole-2-carboxamide (Compound 499)

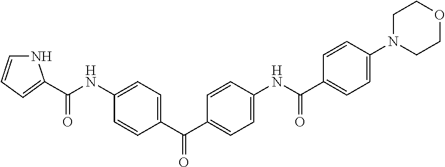 [SMILES: O=C(Nc1ccc(C(=O)c2ccc(NC(=O)c3ccc[nH]3)cc2)cc1)c1ccc(N2CCOCC2)cc1]

Compound 499 was prepared according to the procedure described in Scheme IV from 4,4′-diaminobenzophenone and 4-morpholinobenzoate. [M+H]⁺ calcd for C₂₉H₂₆N₄O₄: 495.06. Found: 495.28.

### Example 400

### 3-Methyl-N-(4-(4-(4-morpholinobenzamido)benzoyl)phenyl)-1H-pyrazole-5-carboxamide (Compound 500)

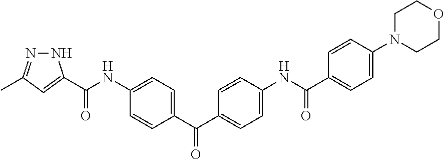 [SMILES: Cc1cc(C(=O)Nc2ccc(C(=O)c3ccc(NC(=O)c4ccc(N5CCOCC5)cc4)cc3)cc2)[nH]n1]

Compound 500 was prepared according to the procedure described in Scheme IV from 4,4′-diaminobenzophenone and 4-morpholinobenzoate. [M+H]⁺ calcd for C₂₉H₂₇N₅O₄: 510.08. Found: 510.06.

### Example 401

### N-(4-(4-(4-Morpholinobenzamido)benzoyl)phenyl)-1H-pyrazole-4-carboxamide (Compound 501)

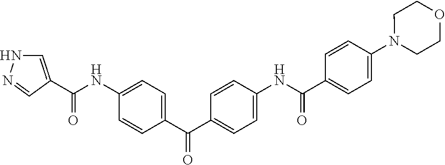 [SMILES: O=C(Nc1ccc(C(=O)c2ccc(NC(=O)c3cn[nH]c3)cc2)cc1)c1ccc(N2CCOCC2)cc1]

Compound 501 was prepared according to the procedure described in Scheme IV from 4,4′-diaminobenzophenone and 4-morpholinobenzoate. [M+H]⁺ calcd for C₂₈H₂₅N₅O₄: 496.05. Found: 496.09.

### Example 402

### N-(4-(4-(4-(4-Hydroxypiperidin-1-yl)benzamido)benzoyl)phenyl)thiophene-2-carboxamide (Compound 502)

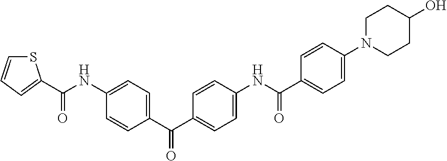 [SMILES: O=C(Nc1ccc(C(=O)c2ccc(NC(=O)c3cccs3)cc2)cc1)c1ccc(N2CCC(O)CC2)cc1]

Compound 502 was prepared according to the procedure described in Scheme IV from 4,4′-diaminobenzophenone and 4-(4-hydroxypiperidinyl)benzoate. [M+H]⁺ calcd for C₃₀H₂₇N₃O₄S: 526.14. Found: 525.99.

### Example 403

### N-(4-(4-(4-Morpholinobenzamido)benzoyl)phenyl)biphenyl-4-carboxamide (Compound 503)

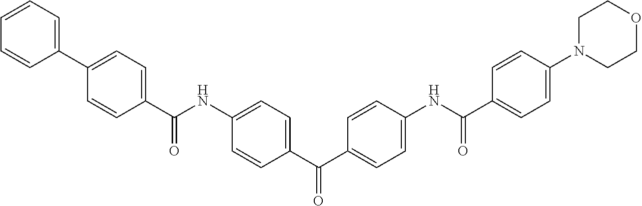 [SMILES: O=C(Nc1ccc(C(=O)c2ccc(NC(=O)c3ccc(N4CCOCC4)cc3)cc2)cc1)c1ccc(-c2ccccc2)cc1]

Compound 503 was prepared according to the procedure described in Scheme IV from 4,4′-diaminobenzophenone and 4-morpholinobenzoate. [M+H]⁺ calcd for C₃₇H₃₁N₃O₄: 582.19. Found: 582.16.

### Example 404

### N-(4-(4-(4-(4-Hydroxypiperidin-1-yl)benzamido)benzoyl)phenyl)pyridine-4-carboxamide (Compound 504)

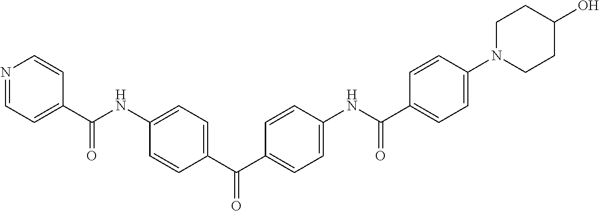 [SMILES: O=C(Nc1ccc(C(=O)c2ccc(NC(=O)c3ccc(N4CCC(O)CC4)cc3)cc2)cc1)c1ccncc1]

Compound 504 was prepared according to the procedure described in Scheme IV from 4,4′-diaminobenzophenone and 4-(4-hydroxypiperidinyl)benzoate. [M+H]⁺ calcd for C₃₁H₂₈N₄O₄: 521.10. Found: 521.06.

### Example 405

### N-(4-(4-(4-(4-Hydroxypiperidin-1-yl)benzamido)benzoyl)phenyl)pyridine-2-carboxamide (Compound 505)

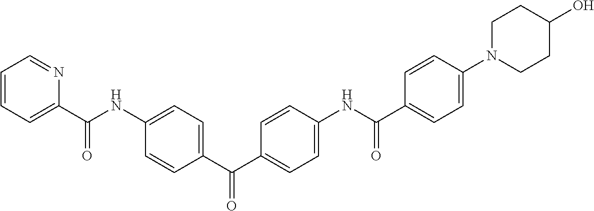 [SMILES: O=C(Nc1ccc(C(=O)c2ccc(NC(=O)c3ccccn3)cc2)cc1)c1ccc(N2CCC(O)CC2)cc1]

Compound 505 was prepared according to the procedure described in Scheme IV from 4,4′-diaminobenzophenone and 4-(4-hydroxypiperidinyl)benzoate. [M+H]⁺ calcd for C₃₁H₂₈N₄O₄: 521.10. Found: 521.06.

### Example 406

### N-(4-(5-(4-(4-Hydroxypiperidin-1-yl)benzamido)-1H-indazol-1-yl)phenyl)-1H-pyrrole-2-carboxamide (Compound 506)

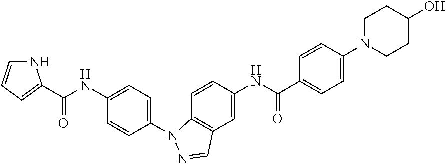 [SMILES: O=C(Nc1ccc2c(cnn2-c2ccc(NC(=O)c3ccc[nH]3)cc2)c1)c1ccc(N2CCC(O)CC2)cc1]

Compound 506 was prepared according to the procedure described in Scheme IV from 5-amino-1-(4-aminophenyl)indazole and 4-(4-hydroxypiperidin-1-yl)benzoate. [M+H]⁺ calcd for C₃₀H₂₈N₆O₃: 521.22. Found: 521.06.

### Example 407

### (±)-N-(4-(5-(4-(3-Aminopyrrolidin-1-yl)benzamido)-1H-indazol-1-yl)phenyl)thiophene-2-carboxamide (Compound 507)

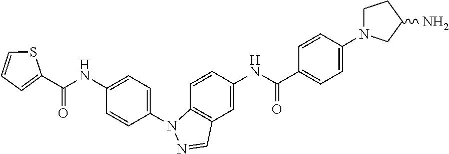 [SMILES: NC1CCN(c2ccc(C(=O)Nc3ccc4c(cnn4-c4ccc(NC(=O)c5cccs5)cc4)c3)cc2)C1]

Compound 507 was prepared according to the procedure described in Scheme IV from 5-amino-1-(4-aminophenyl)indazole and 4-(3-aminopyrrolidin-1-yl)benzoate. [M+H]⁺ calcd for C₂₉H₂₇N₆O₂S: 523.19. Found: 523.02.

### Example 408

### N-(4-(5-(4-(4-Hydroxypiperidin-1-yl)benzamido)-1H-indazol-1-yl)phenyl)-1H-pyrazole-3-carboxamide (Compound 508)

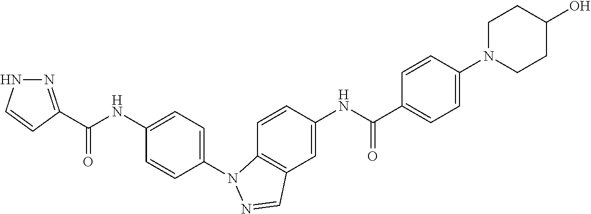 [SMILES: O=C(Nc1ccc2c(cnn2-c2ccc(NC(=O)c3cc[nH]n3)cc2)c1)c1ccc(N2CCC(O)CC2)cc1]

Compound 508 was prepared according to the procedure described in Scheme IV from 5-amino-1-(4-aminophenyl)indazole and 4-(4-hydroxypiperidin-1-yl)benzoate. [M+H]⁺ calcd for C₂₉H₂₇N₇O₃: 522.22. Found: 522.05.

### Example 409

### N-(4-(5-(4-(4-Hydroxymethylpiperidin-1-yl)benzamido)-1H-indazol-1-yl)phenyl)thiophene-2-carboxamide (Compound 509)

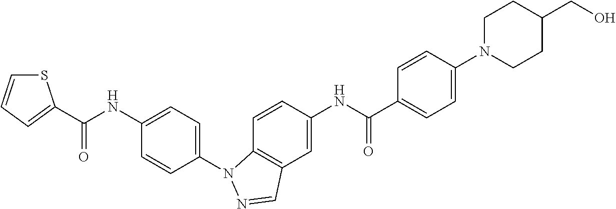 [SMILES: O=C(Nc1ccc2c(cnn2-c2ccc(NC(=O)c3cccs3)cc2)c1)c1ccc(N2CCC(CO)CC2)cc1]

Compound 509 was prepared according to the procedure described in Scheme IV from 5-amino-1-(4-aminophenyl)indazole and 4-(4-hydroxymethylpiperidin-1-yl)benzoate. [M+H]⁺ calcd for C₃₁H₂₉N₅O₃S: 552.20. Found: 552.05.

### Example 410

### N-(4-(5-(4-(4-Hydroxypiperidin-1-yl)benzamido)-1H-indazol-1-yl)phenyl)-1H-indole-6-carboxamide (Compound 510)

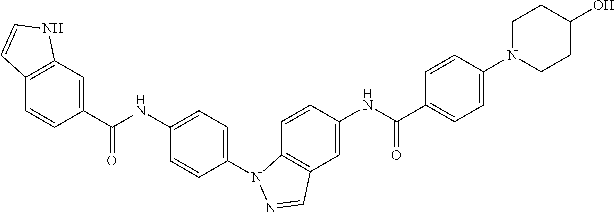 [SMILES: O=C(Nc1ccc2c(cnn2-c2ccc(NC(=O)c3ccc4cc[nH]c4c3)cc2)c1)c1ccc(N2CCC(O)CC2)cc1]

Compound 510 was prepared according to the procedure described in Scheme IV from 5-amino-1-(4-aminophenyl)indazole and 4-(4-hydroxypiperidin-1-yl)benzoate. [M+H]⁺ calcd for C₃₄H₃₀N₆O₃: 571.24. Found: 571.16.

### Example 411

### N-(4-(5-(4-(4-Hydroxypiperidin-1-yl)benzamido)-1H-indazol-1-yl)phenyl)-1H-indole-5-carboxamide (Compound 511)

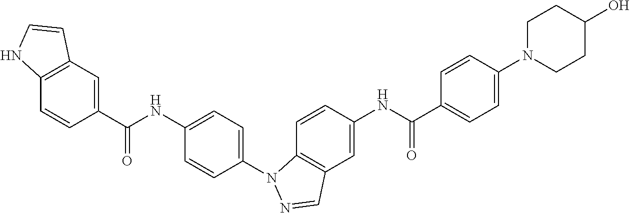 [SMILES: O=C(Nc1ccc2c(cnn2-c2ccc(NC(=O)c3ccc4[nH]ccc4c3)cc2)c1)c1ccc(N2CCC(O)CC2)cc1]

Compound 511 was prepared according to the procedure described in Scheme IV from 5-amino-1-(4-aminophenyl)indazole and 4-(4-hydroxypiperidin-1-yl)benzoate. [M+H]⁺ calcd for C₃₄H₃₀N₆O₃: 571.24. Found: 571.16.

### Example 412

### N-(4-(5-(4-(4-(2-Hydroxyethyl)piperidin-1-yl)benzamido)-1H-indazol-1-yl)phenyl)thiophene-2-carboxamide (Compound 512)

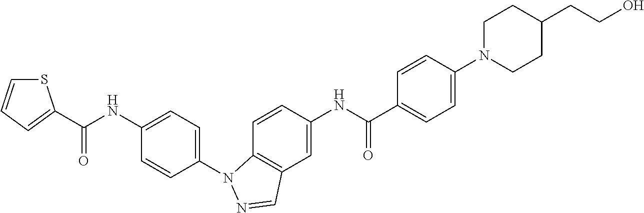 [SMILES: O=C(Nc1ccc2c(cnn2-c2ccc(NC(=O)c3cccs3)cc2)c1)c1ccc(N2CCC(CCO)CC2)cc1]

Compound 512 was prepared according to the procedure described in Scheme IV from 5-amino-1-(4-aminophenyl)indazole and 4-(4-(2-hydroxyethyl)piperidin-1-yl)benzoate. [M+H]⁺ calcd for C₃₂H₃₁N₅O₃S: 566.21. Found: 566.11.

### Example 413

### N-(4-(5-(4-(4-Cyclopropylcarbonylpiperiazin-1-yl)benzamido)-1H-indazol-1-yl)phenyl)thiophene-2-carboxamide (Compound 513)

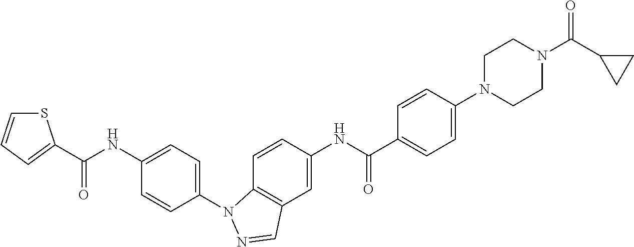 [SMILES: O=C(Nc1ccc2c(cnn2-c2ccc(NC(=O)c3cccs3)cc2)c1)c1ccc(N2CCN(C(=O)C3CC3)CC2)cc1]

Compound 513 was prepared according to the procedure described in Scheme IV from 5-amino-1-(4-aminophenyl)indazole and 4-(4-cyclopropylcarbonylpiperazin-1-yl)benzoate. [M+H]⁺ calcd for C₃₃H₃₀N₆O₃S: 591.21. Found: 591.14.

### Example 414

### N-(4-(5-(4-(4-Mopholinopiperidin-1-yl)benzamido)-1H-indazol-1-yl)phenyl)thiophene-2-carboxamide (Compound 514)

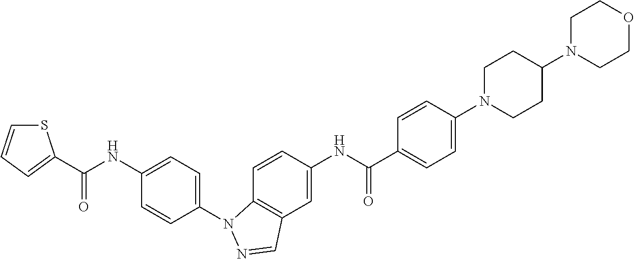 [SMILES: O=C(Nc1ccc2c(cnn2-c2ccc(NC(=O)c3cccs3)cc2)c1)c1ccc(N2CCC(N3CCOCC3)CC2)cc1]

Compound 514 was prepared according to the procedure described in Scheme IV from 5-amino-1-(4-aminophenyl)indazole and 4-(4-morpholinopiperidin-1-yl)benzoate. [M+H]⁺ calcd for C₃₄H₃₅N₆O₃S: 607.25. Found: 607.19.

### Example 415

### N-(1-(4-Aminophenyl)-1H-indazol-5-yl)-4-(4-hydroxypiperidin-1-yl)benzamide (Compound 515)

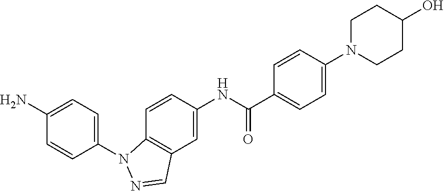 [SMILES: Nc1ccc(-n2ncc3cc(NC(=O)c4ccc(N5CCC(O)CC5)cc4)ccc32)cc1]

Compound 515 was prepared according to the procedure described in Scheme IV from 5-amino-1-(4-aminophenyl)indazole and 4-(4-hydroxypiperidin-1-yl)benzoate. [M+H]⁺ calcd for C₂₅H₂₅N₅O₂: 428.20. Found: 427.93.

### Example 416

### N-(4-(5-(4-(4-((1H-Imidazol-1-yl)methyl)-4-hydroxypiperidin-1-yl)benzamido)-1H-indazol-1-yl)phenyl)thiophene-2-carboxamide (Compound 516)

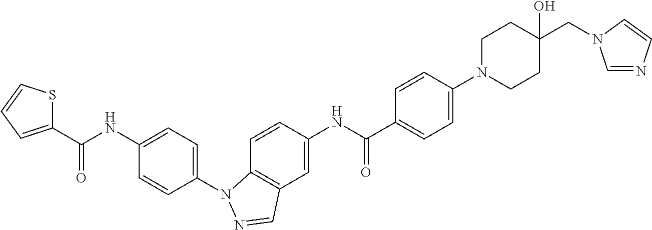 [SMILES: O=C(Nc1ccc2c(cnn2-c2ccc(NC(=O)c3cccs3)cc2)c1)c1ccc(N2CCC(O)(Cn3ccnc3)CC2)cc1]

Compound 516 was prepared according to the procedure described in Scheme IV from 5-amino-1-(4-aminophenyl)indazole and 4-(4-((1H-imidazol-1-yl)methyl)-4-hydroxypiperidin-1-yl)benzoate. [M+H]⁺ calcd for C₃₄H₃₁N₇O₃S: 618.22. Found: 618.15.

### Example 417

### 4-Cyano-N-(4-(5-(4-mopholinobenzamido)-1H-indazol-1-yl)phenyl)benzamide (Compound 517)

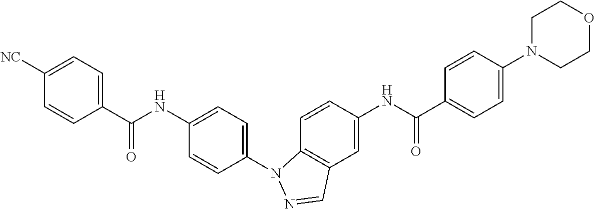 [SMILES: N#Cc1ccc(C(=O)Nc2ccc(-n3ncc4cc(NC(=O)c5ccc(N6CCOCC6)cc5)ccc43)cc2)cc1]

Compound 517 was prepared according to the procedure described in Scheme IV from 5-amino-1-(4-aminophenyl)indazole and 4-morpholinobenzoate. [M+H]⁺ calcd for C₃₂H₂₆N₆O₃: 543.21. Found: 543.08.

### Example 418

### N-(4-(5-(4-(1,4-Dioxa-8-azaspiro[4,5]decan-8-yl)benzamido)-1H-indazol-1-yl)phenyl)thiophene-2-carboxamide (Compound 518)

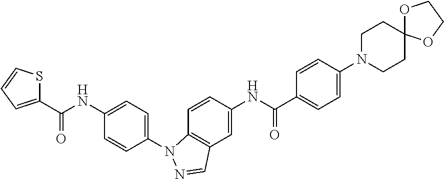 [SMILES: O=C(Nc1ccc2c(cnn2-c2ccc(NC(=O)c3cccs3)cc2)c1)c1ccc(N2CCC3(CC2)OCCO3)cc1]

Compound 518 was prepared according to the procedure described in Scheme IV from 5-amino-1-(4-aminophenyl)indazole and 4-(1,4-dioxa-8-azaspiro[4,5]decan-8-yl)benzoate. [M+H]⁺ calcd for C₃₂H₂₉N₅O₄S: 580.19. Found: 580.07.

### Example 419

### N-(4-(5-(4-Fluorobenzamido)-1H-indazol-1-yl)phenyl)-1H-indole-5-carboxamide (Compound 519)

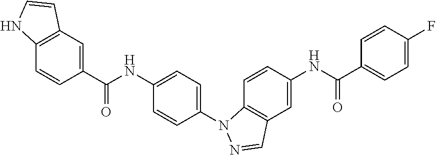 [SMILES: O=C(Nc1ccc2c(cnn2-c2ccc(NC(=O)c3ccc4[nH]ccc4c3)cc2)c1)c1ccc(F)cc1]

Compound 519 was prepared according to the procedure described in Scheme IV from 5-amino-(4-aminophenyl)indazole and 4-fluorobenzoate. [M+H]⁺ calcd for C₂₉H₂₀FN₅O₂: 490.16. Found: 490.01.

### Example 420

### (±)-N-(4-(5-(4-(3-Hydroxymethyl)piperidin-1-yl)benzamido)-1H-indazol-1-yl)phenyl)thiophene-2-carboxamide (Compound 520)

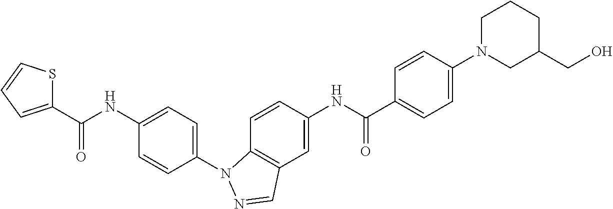 [SMILES: O=C(Nc1ccc2c(cnn2-c2ccc(NC(=O)c3cccs3)cc2)c1)c1ccc(N2CCCC(CO)C2)cc1]

Compound 520 was prepared according to the procedure described in Scheme IV from 5-amino-1-(4-aminophenyl)indazole and 4-(3-hydroxymethyl)piperidin-1-yl)benzoate. [M+H]⁺ calcd for C₃₁H₂₉N₅O₃S: 552.20. Found: 552.07.

### Example 421

### (±)-N-(1-(4-(4-(Hexahydropyrrolo[1,2-a]pyrazin-2(1H)-yl)benzamido)phenyl)-1H-indazol-5-yl)thiophene-2-carboxamide (Compound 521)

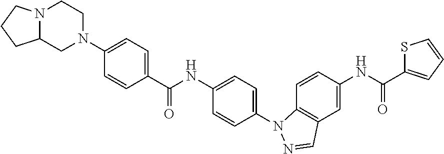 [SMILES: O=C(Nc1ccc(-n2ncc3cc(NC(=O)c4cccs4)ccc32)cc1)c1ccc(N2CCN3CCCC3C2)cc1]

Compound 521 was prepared according to the procedure described in Scheme IV from 5-amino-(4-aminophenyl)indazole and 4-(hexahydropyrrolo[1,2-a]pyrazin-2(1H)-yl)benzoate. [M+H]⁺ calcd for C₃₂H₃₀N₆O₂S: 563.22. Found: 563.12.

### Example 422

### N-(1-(4-(4-(4-Dimethylaminopiperidin-1-yl)benzamido)phenyl)-1H-indazol-5-yl)thiophene-2-carboxamide (Compound 522)

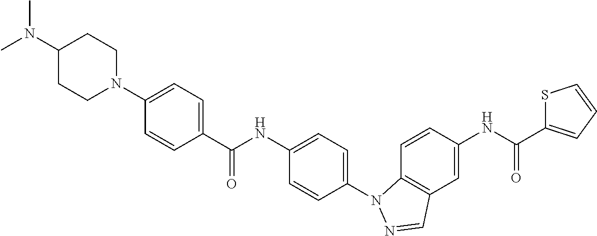 [SMILES: CN(C)C1CCN(c2ccc(C(=O)Nc3ccc(-n4ncc5cc(NC(=O)c6cccs6)ccc54)cc3)cc2)CC1]

Compound 522 was prepared according to the procedure described in Scheme IV from 5-amino-(4-aminophenyl)indazole and 4-(4-dimethylaminopiperidin-1-yl))benzoate. [M+H]⁺ calcd for C₃₂H₃₂N₆O₂S: 565.23. Found: 565.14.

### Example 423

### N-(4-(5-(1H-Indole-6-carboxamido)-1H-indazol-1-yl)phenyl)-1H-indole-6-carboxamide (Compound 523)

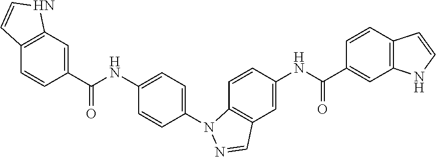 [SMILES: O=C(Nc1ccc(-n2ncc3cc(NC(=O)c4ccc5cc[nH]c5c4)ccc32)cc1)c1ccc2cc[nH]c2c1]

Compound 523 was prepared according to the procedure described in Scheme IV from 5-amino-(4-aminophenyl)indazole and 1H-indole-6-carboxylic acid. [M+H]⁺ calcd for C₃₁H₂₂N₆O₂: 511.18. Found: 511.02.

### Example 424

### (±)-N-(1-(4-(4-(3-Dimethylaminopyrrolidin-1-yl)benzamido)phenyl)-1H-indazol-5-yl)thiophene-2-carboxamide (Compound 524)

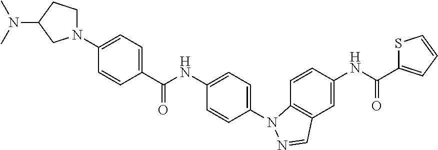 [SMILES: CN(C)C1CCN(c2ccc(C(=O)Nc3ccc(-n4ncc5cc(NC(=O)c6cccs6)ccc54)cc3)cc2)C1]

Compound 524 was prepared according to the procedure described in Scheme IV from 5-amino-(4-aminophenyl)indazole and 4-(3-dimethylaminopyrrolidin-1-yl)benzoate. [M+H]⁺ calcd for C₃₁H₃₀N₆O₂S: 551.22. Found: 551.10.

### Example 425

### (±)-N-(1-(4-(4-((2-Hydroxyethyl)methylamino)benzamido)phenyl)-1H-indazol-5-yl)thiophene-2-carboxamide (Compound 525)

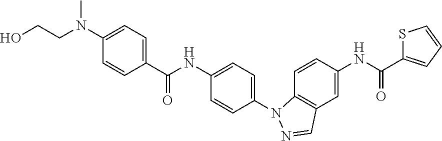 [SMILES: CN(CCO)c1ccc(C(=O)Nc2ccc(-n3ncc4cc(NC(=O)c5cccs5)ccc43)cc2)cc1]

Compound 525 was prepared according to the procedure described in Scheme IV from 5-amino-1-(4-aminophenyl)indazole and 4-((2-hydroxyethyl)methylamino)benzoate. [M+H]⁺ calcd for C₂₈H₂₅N₅O₃S: 512.17. Found: 512.02.

### Example 426

### N-(1-(4-(4-(4-Hydroxypiperidin-1-yl)benzamido)phenyl)-1H-indazol-5-yl)thiophene-2-carboxamide (Compound 526)

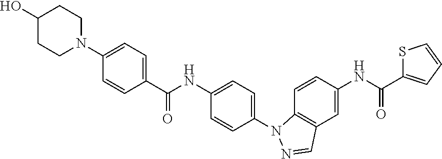 [SMILES: O=C(Nc1ccc(-n2ncc3cc(NC(=O)c4cccs4)ccc32)cc1)c1ccc(N2CCC(O)CC2)cc1]

Compound 526 was prepared according to the procedure described in Scheme IV from 5-amino-1-(4-aminophenyl)indazole and 4-(4-hydroxypiperidin-1-yl))benzoate. [M+H]⁺ calcd for C₃₀H₂₇N₅O₃S: 538.18. Found: 538.06.

### Example 427

### N-(4-(5-(1H-Indole-5-carboxamido)-1H-indazol-1-yl)phenyl)-1H-indole-5-carboxamide (Compound 527)

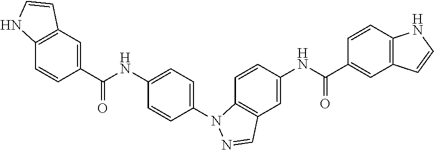 [SMILES: O=C(Nc1ccc(-n2ncc3cc(NC(=O)c4ccc5[nH]ccc5c4)ccc32)cc1)c1ccc2[nH]ccc2c1]

Compound 527 was prepared according to the procedure described in Scheme IV from 5-amino-1-(4-aminophenyl)indazole and 1H-indole-5-carboxylic acid. [M+H]⁺ calcd for C₃₁H₂₂N₆O₂: 511.18. Found: 511.03.

### Example 428

### N-(4-(5-(4-(4-Hydroxypiperidin-1-yl)benzamido)-1H-indazol-1-yl)phenyl)thiophene-2-carboxamide (Compound 528)

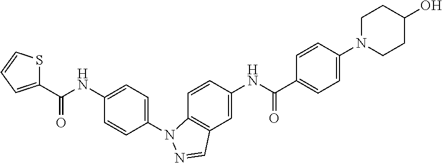 [SMILES: O=C(Nc1ccc2c(cnn2-c2ccc(NC(=O)c3cccs3)cc2)c1)c1ccc(N2CCC(O)CC2)cc1]

Compound 528 was prepared according to the procedure described in Scheme IV from 5-amino-1-(4-aminophenyl)indazole and 4-(4-hydroxypiperidin-1-yl)benzoate. [M+H]⁺ calcd for C₃₀H₂₇N₅O₃S: 538.18. Found: 538.06.

### Example 429

### 4-Morpholino-N-(4-(5-(4-morpholinobenzamido)-1H-indazol-1-yl)phenyl)piperidine-1-carboxamide (Compound 529)

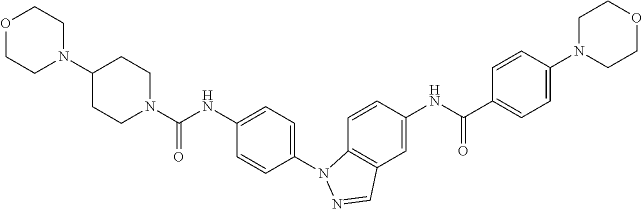 [SMILES: O=C(Nc1ccc2c(cnn2-c2ccc(NC(=O)N3CCC(N4CCOCC4)CC3)cc2)c1)c1ccc(N2CCOCC2)cc1]

Compound 529 was prepared according to the procedure described in Scheme IV from 5-amino-1-(4-aminophenyl)indazole and 4-morpholinobenzoate. [M+H]⁺ calcd for C₃₄H₃₉N₇O₄: 610.31. Found: 610.25.

### Example 430

### N-(1-(4-Cyclopropylmethylamino)phenyl)-1H-indazol-5-yl)-4-morpholinobenzamide (Compound 530)

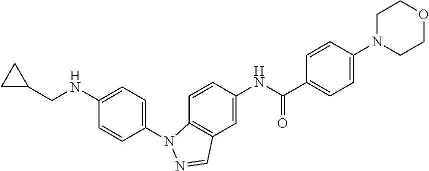 [SMILES: O=C(Nc1ccc2c(cnn2-c2ccc(NCC3CC3)cc2)c1)c1ccc(N2CCOCC2)cc1]

Compound 530 was prepared according to the procedure described in Scheme IV from 5-amino-1-(4-aminophenyl)indazole and 4-morpholinobenzoate. [M+H]⁺ calcd for C₂₈H₂₉N₅O₂: 468.23. Found: 468.08.

### Example 431

### N-(1-(4-Ethylamino)phenyl)-1H-indazol-5-yl)-4-morpholinobenzamide (Compound 531)

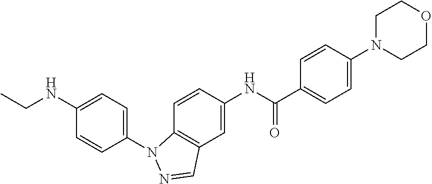 [SMILES: CCNc1ccc(-n2ncc3cc(NC(=O)c4ccc(N5CCOCC5)cc4)ccc32)cc1]

Compound 531 was prepared according to the procedure described in Scheme IV from 5-amino-1-(4-aminophenyl)indazole and 4-morpholinobenzoate. [M+H]⁺ calcd for C₂₆H₂₇N₅O₂: 442.22. Found: 442.05.

### Example 432

### Phenyl 4-(5-(4-morpholinobenzamido)-1H-indazol-5-yl)phenylcarbamate (Compound 532)

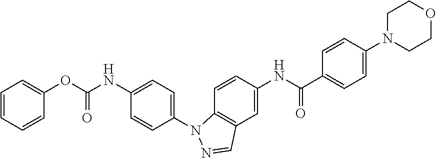 [SMILES: O=C(Nc1ccc(-n2ncc3cc(NC(=O)c4ccc(N5CCOCC5)cc4)ccc32)cc1)Oc1ccccc1]

Compound 532 was prepared according to the procedure described in Scheme IV from 5-amino-1-(4-aminophenyl)indazole and 4-morpholinobenzoate. ¹H NMR (500 MHz, DMSO-d₆) δ 10.49 (s, 1H), 9.97 (s, 1H), 8.97 (s, 1H), 8.03 (d, J=7.5 Hz, 2H), 7.91 (d, J=8 Hz, 2H), 7.68 (m, 3H), 7.56 (d, J=9 Hz, 1H), 7.44 (t, J=7.5 Hz, 2H), 7.26 (m, 3H), 7.03 (d, J=7.5 Hz, 2H), 3.74 (m, 4H).

### Example 433

### N-(4-(5-(4-morpholinobenzamido)-1H-indazol-5-yl)phenyl)morpholine-4-carboxamide (Compound 533)

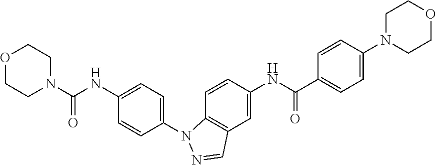 [SMILES: O=C(Nc1ccc2c(cnn2-c2ccc(NC(=O)N3CCOCC3)cc2)c1)c1ccc(N2CCOCC2)cc1]

Compound 533 was prepared according to the procedure described in Scheme IV from 5-amino-1-(4-aminophenyl)indazole and 4-morpholinobenzoate. [M+H]⁺ calcd for C₂₉H₃₀N₆O₄: 527.23. Found: 527.00.

### Example 434

### N-(1-(4-Cyclopropanecarboxamido)phenyl)-2-oxoindolin-5-yl)4-dimethylaminobenzamide (Compound 534)

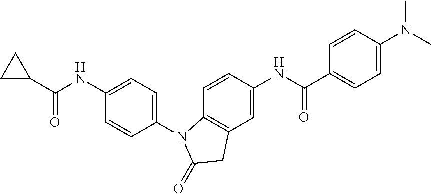 [SMILES: CN(C)c1ccc(C(=O)Nc2ccc3c(c2)CC(=O)N3c2ccc(NC(=O)C3CC3)cc2)cc1]

Compound 534 was prepared according to the procedure described in Scheme IV from 5-amino-1-(4-aminophenyl)oxindole and 4-dimethylaminobenzoate. [M+H]⁺ calcd for C₂₇H₂₆N₄O₃: 455.20. Found: 455.00.

### Example 435

### 4-Morpholino-N-(1-(4-(thiazol-2-ylmethylamino)phenyl)-1H-indazol-5-yl)benzamide (Compound 535)

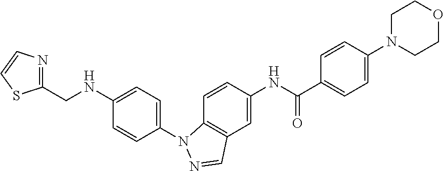 [SMILES: O=C(Nc1ccc2c(cnn2-c2ccc(NCc3nccs3)cc2)c1)c1ccc(N2CCOCC2)cc1]

Compound 535 was prepared according to the procedure described in Scheme IV from 5-amino-1-(4-aminophenyl)indazole and 4-morpholinobenzoate. ¹H NMR (500 MHz, CDCl₃) δ 8.17 (s, 1H), 8.12 (s, 1H), 7.85 (m, 3H), 7.78 (m, 1H), 7.59 (d, J=9 Hz, 1H), 7.49 (d, J=8 Hz, 1H), 7.30 (m, 1H), 6.95 (d, J=8 Hz, 2H), 6.82 (d, J=8.5 Hz, 2H), 4.76 (s, 2H), 3.89 (t, J=4 Hz, 4H), 3.29 (t, J=4.5 Hz, 4H).

### Example 436

### 4-Morpholino-N-(1-(4-(thiazol-2-ylmethylideneamino)phenyl)-1H-indazol-5-yl)benzamide (Compound 536)

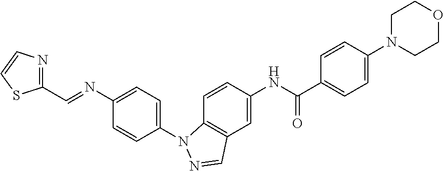 [SMILES: O=C(Nc1ccc2c(cnn2-c2ccc(/N=C/c3nccs3)cc2)c1)c1ccc(N2CCOCC2)cc1]

Compound 536 was prepared according to the procedure described in Scheme IV from 5-amino-1-(4-aminophenyl)indazole and 4-morpholinobenzoate. No data.

### Example 437

### 4-Morpholino-N-(1-(4-morpholinophenyl)-1H-indazol-5-yl)benzamide (Compound 537)

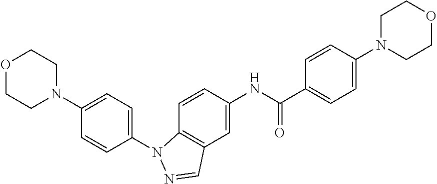 [SMILES: O=C(Nc1ccc2c(cnn2-c2ccc(N3CCOCC3)cc2)c1)c1ccc(N2CCOCC2)cc1]

Compound 537 was prepared according to the procedure described in Scheme IV from 5-amino-1-(4-aminophenyl)indazole and 4-morpholinobenzoate. [M+H]⁺ calcd for C₂₈H₂₉N₅O₃: 484.23. Found: 484.11.

### Example 438

### 4-Morpholino-N-(2-(4-morpholinophenyl)-2H-indazol-5-yl)benzamide (Compound 538)

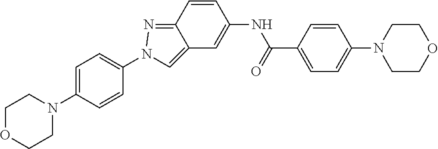 [SMILES: O=C(Nc1ccc2nn(-c3ccc(N4CCOCC4)cc3)cc2c1)c1ccc(N2CCOCC2)cc1]

Compound 538 was prepared according to the procedure described in Scheme IV from 5-amino-2-(4-aminophenyl)indazole and 4-morpholinobenzoate. [M+H]⁺ calcd for C₂₈H₂₉N₅O₃: 484.23. Found: 484.13.

### Example 439

### (±)-N-(4-(5-(4-(3-Hydroxypyrrolidin-1-yl)benzamido)-1H-indazol-1-yl)phenyl)thiophene-2-carboxamide (Compound 539)

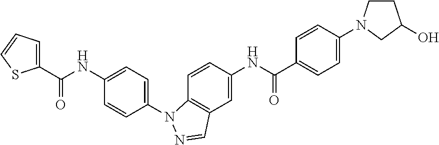 [SMILES: O=C(Nc1ccc2c(cnn2-c2ccc(NC(=O)c3cccs3)cc2)c1)c1ccc(N2CCC(O)C2)cc1]

Compound 539 was prepared according to the procedure described in Scheme IV from 5-amino-1-(4-aminophenyl)indazole and 4-(3-hydroxypyrrolidinyl)benzoate. [M+H]⁺ calcd for C₂₉H₂₅N₅O₃S: 524.17. Found: 524.04.

### Example 440

### 4-Morpholino-N-(2-(4-nitrophenyl)-2H-indazol-5-yl)benzamide (Compound 540)

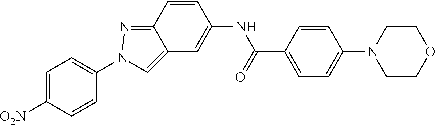 [SMILES: O=C(Nc1ccc2nn(-c3ccc([N+](=O)[O-])cc3)cc2c1)c1ccc(N2CCOCC2)cc1]

Compound 540 was prepared according to the procedure described in Scheme IV from 5-amino-2-(4-nitrophenyl)indazole and 4-morpholinobenzoate. [M+H]⁺ calcd for C₂₄H₂₁N₅O₄: 444.16. Found: 444.27.

### Example 441

### 4-Morpholino-N-(1-(4-(3-(2-morpholinoethyl)ureido)phenyl)-1H-indazol-5-yl)phenyl)benzamide (Compound 541)

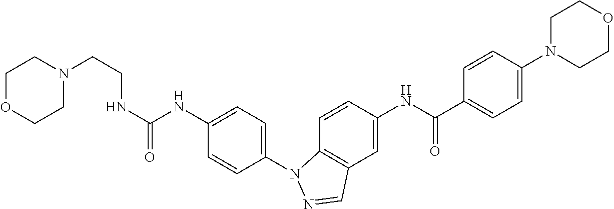 [SMILES: O=C(NCCN1CCOCC1)Nc1ccc(-n2ncc3cc(NC(=O)c4ccc(N5CCOCC5)cc4)ccc32)cc1]

Compound 541 was prepared according to the procedure described in Scheme IV from 5-amino-1-(4-aminophenyl)indazole and 4-morpholinobenzoate. [M+H]⁺ calcd for C₃₁H₃₅N₇O₄: 570.28. Found: 570.21.

### Example 442

### 4-Morpholino-N-(2-(4-acetylphenyl)-2H-indazol-5-yl)benzamide (Compound 542)

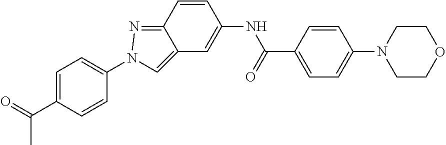 [SMILES: CC(=O)c1ccc(-n2cc3cc(NC(=O)c4ccc(N5CCOCC5)cc4)ccc3n2)cc1]

Compound 542 was prepared according to the procedure described in Scheme IV from 5-amino-2-(4-acetylphenyl)indazole and 4-morpholinobenzoate. [M+H]⁺ calcd for C₂₆H₂₄N₄O₃: 441.18. Found: 441.02.

### Example 443

### N-(2-(4-Cyanophenyl)-2H-indazol-5-yl)-4-(4-hydroxypiperidin-1-yl)benzamide (Compound 543)

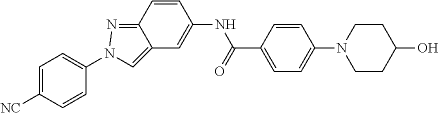 [SMILES: N#Cc1ccc(-n2cc3cc(NC(=O)c4ccc(N5CCC(O)CC5)cc4)ccc3n2)cc1]

Compound 543 was prepared according to the procedure described in Scheme IV from 5-amino-2-(4-acetylphenyl)indazole and 4-(4-hydroxypiperidin-1-yl)benzoate. [M+H]⁺ calcd for C₂₆H₂₃N₅O₂: 438.19. Found: 438.05.

### Example 444

### N-(4-(5-(Cyclopropanecarboxamido)-1H-indazol-1-yl)phenyl)-4-morpholinobenzamide (Compound 544)

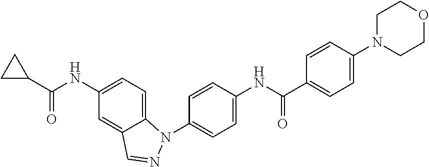 [SMILES: O=C(Nc1ccc(-n2ncc3cc(NC(=O)C4CC4)ccc32)cc1)c1ccc(N2CCOCC2)cc1]

Compound 544 was prepared according to the procedure described in Scheme IV from 5-amino-1-(4-aminophenyl)indazole and 4-morpholinobenzoate. [M+H]⁺ calcd for C₂₈H₂₇N₅O₃: 482.21. Found: 482.05.

### Example 445

### N-(4-(5-(4-Morpholinobenzamido)-1H-indazol-1-yl)phenyl)-4-oxopiperidine-1-carboxamide (Compound 545)

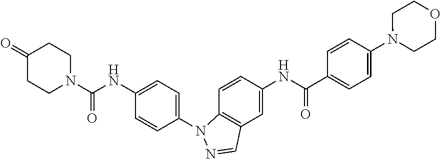 [SMILES: O=C1CCN(C(=O)Nc2ccc(-n3ncc4cc(NC(=O)c5ccc(N6CCOCC6)cc5)ccc43)cc2)CC1]

Compound 545 was prepared according to the procedure described in Scheme IV from 5-amino-1-(4-aminophenyl)indazole and 4-morpholinobenzoate. [M+H]⁺ calcd for C₃₀H₃₀N₆O₄: 539.23. Found: 539.12.

### Example 446

### 4-Methyl-N-(4-(5-(4-morpholinobenzamido)-1H-indazol-1-yl)phenyl)thiazole-5-carboxamide (Compound 546)

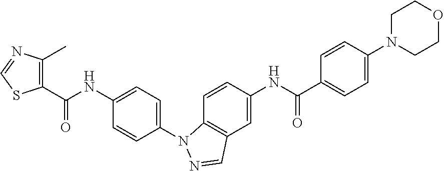 [SMILES: Cc1ncsc1C(=O)Nc1ccc(-n2ncc3cc(NC(=O)c4ccc(N5CCOCC5)cc4)ccc32)cc1]

Compound 546 was prepared according to the procedure described in Scheme IV from 5-amino-1-(4-aminophenyl)indazole and 4-morpholinobenzoate. [M+H]⁺ calcd for C₂₉H₂₆N₆O₃S: 539.19. Found: 539.09.

### Example 447

### N-(4-(5-(4-Morpholinobenzamido)-1H-indazol-1-yl)phenyl)thiazole-5-carboxamide (Compound 547)

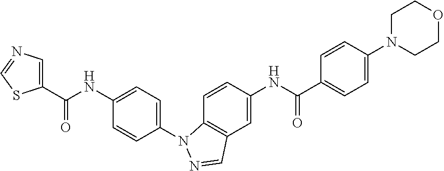 [SMILES: O=C(Nc1ccc2c(cnn2-c2ccc(NC(=O)c3cncs3)cc2)c1)c1ccc(N2CCOCC2)cc1]

Compound 547 was prepared according to the procedure described in Scheme IV from 5-amino-1-(4-aminophenyl)indazole and 4-morpholinobenzoate. ¹H NMR (500 MHz, DMSO-d₆) 10.66 (s, 1H), 10.12 (s, 1H), 9.34 (s, 1H), 8.75 (s, 1H), 8.40 (s, 1H), 8.36 (s, 1H), 7.94 (dd, J=3, 9 Hz, 4H), 7.87 (d, J=9 Hz, 1H), 7.81 (m, 3H), 7.05 (d, J=9 Hz, 2H), 3.76 (m, 4H).

### Example 448

### N-(4-(5-(4-Morpholinobenzamido)-1H-indazol-1-yl)phenyl)thiazole-2-carboxamide (Compound 548)

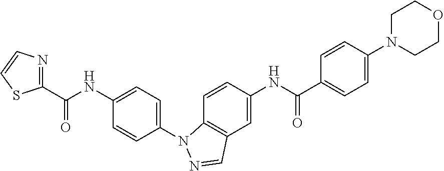 [SMILES: O=C(Nc1ccc2c(cnn2-c2ccc(NC(=O)c3nccs3)cc2)c1)c1ccc(N2CCOCC2)cc1]

Compound 548 was prepared according to the procedure described in Scheme IV from 5-amino-1-(4-aminophenyl)indazole and 4-morpholinobenzoate. [M+H]⁺ calcd for C₂₈H₂₄N₆O₃S: 525.16. Found: 525.04.

### Example 449

### N-(1-(4-(1-(Hydroxyimino)ethyl)pheyl)-1H-indazol-5-yl)morpholinobenzamide (Compound 549)

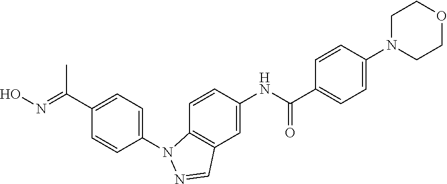 [SMILES: C/C(=N\O)c1ccc(-n2ncc3cc(NC(=O)c4ccc(N5CCOCC5)cc4)ccc32)cc1]

Compound 549 was prepared according to the procedure described in Scheme IV from 5-amino-1-(4-acetylphenyl)indazole and 4-morpholinobenzoate. [M+H]⁺ calcd for C₂₆H₂₅N₅O₃: 456.20. Found: 456.07.

### Example 450

### 4-Methyl-N-(4-(5-(4-morpholinobenzamido)-1H-indazol-1-yl)phenyl)piperazine-1-carboxamide (Compound 550)

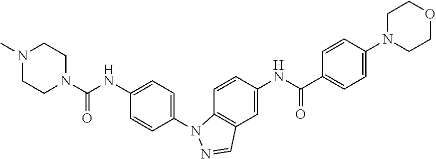 [SMILES: CN1CCN(C(=O)Nc2ccc(-n3ncc4cc(NC(=O)c5ccc(N6CCOCC6)cc5)ccc43)cc2)CC1]

Compound 550 was prepared according to the procedure described in Scheme IV from 5-amino-1-(4-aminophenyl)indazole and 4-morpholinobenzoate. [M+H]⁺ calcd for C₃₀H₃₃N₇O₃: 540.26. Found: 540.15.

### Example 451

### (±)-N-(4-(5-(4-(3-Hydroxypiperidin-1-yl)benzamido)-1H-indazol-1-yl)phenyl)thiophene-2-carboxamide (Compound 551)

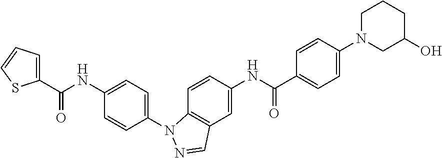 [SMILES: O=C(Nc1ccc2c(cnn2-c2ccc(NC(=O)c3cccs3)cc2)c1)c1ccc(N2CCCC(O)C2)cc1]

Compound 551 was prepared according to the procedure described in Scheme IV from 5-amino-1-(4-aminophenyl)indazole and 4-(3-hydroxypiperidinyl)benzoate. [M+H]⁺ calcd for C₃₀H₂₇N₅O₃S: 538.18. Found: 538.06.

### Example 452

### N-(1-(4-Cyclopropanecarboxamido)phenyl)-1H-indazol-5-yl)-2-morpholinopyrimidine-5-carboxamide (Compound 552)

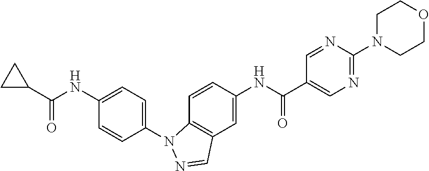 [SMILES: O=C(Nc1ccc2c(cnn2-c2ccc(NC(=O)C3CC3)cc2)c1)c1cnc(N2CCOCC2)nc1]

Compound 552 was prepared according to the procedure described in Scheme IV from 5-amino-1-(4-aminophenyl)indazole and 2-morpholino-5-pyrimidinecarboxylate. [M+H]⁺ calcd for C₂₆H₂₅N₇O₃: 484.20. Found: 484.01.

### Example 453

### N-(1-(4-Aminophenyl)-1H-indazol-5-yl)-4-(1H-imidazol-1-yl)benzamide (Compound 553)

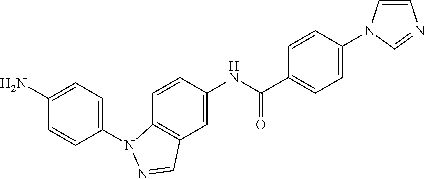 [SMILES: Nc1ccc(-n2ncc3cc(NC(=O)c4ccc(-n5ccnc5)cc4)ccc32)cc1]

Compound 553 was prepared according to the procedure described in Scheme IV from 5-amino-1-(4-aminophenyl)indazole and 4-imidazolylbenzoate. ¹H NMR (500 MHz, Acetone-d₆) δ 9.78 (s, 1H), 8.45 (s, 1H), 8.20 (m, 4H), 7.82-7.67 (m, 5H), 7.42 (d, J=8.5 Hz, 2H), 7.16 (s, 1H), 6.87 (d, J=8.5 Hz, 2H), 4.93 (s, 2H).

### Example 454

### N-(4-(5-(4-(1H-Imidazol-1-yl)benzamido)-1H-indazol-1-yl)phenyl)thiophene-2-carboxamide (Compound 554)

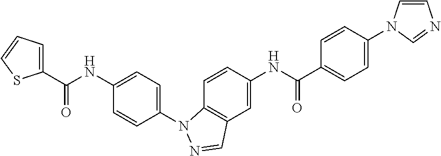 [SMILES: O=C(Nc1ccc2c(cnn2-c2ccc(NC(=O)c3cccs3)cc2)c1)c1ccc(-n2ccnc2)cc1]

Compound 554 was prepared according to the procedure described in Scheme IV from 5-amino-1-(4-aminophenyl)indazole and 4-imidazolylbenzoate. [M+H]calcd for C₂₈H₂₀N₆O₂S: 505.15. Found: 505.00.

### Example 455

### N-(4-(5-(4-(4-Hydroxypiperidin-1-yl)benzamido)-1H-indazol-1-yl)phenyl)pyrimidine-5-carboxamide (Compound 555)

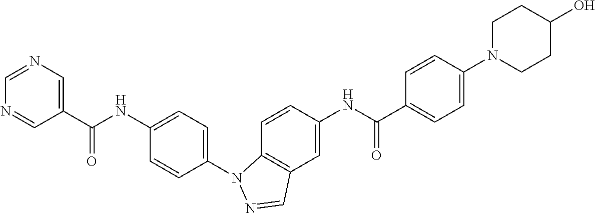 [SMILES: O=C(Nc1ccc2c(cnn2-c2ccc(NC(=O)c3cncnc3)cc2)c1)c1ccc(N2CCC(O)CC2)cc1]

Compound 555 was prepared according to the procedure described in Scheme IV from 5-amino-1-(4-aminophenyl)indazole and 4-(4-hydroxypiperidin-1-yl)benzoate. [M+H]⁺ calcd for C₃₀H₂₇N₇O₃: 534.22. Found: 534.09.

### Example 456

### N-(4-(5-(4-(Tetrahydropyran-4-yl)amino)benzamido)-1H-indazol-1-yl)phenyl)thiophene-2-carboxamide (Compound 556)

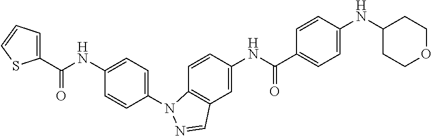 [SMILES: O=C(Nc1ccc2c(cnn2-c2ccc(NC(=O)c3cccs3)cc2)c1)c1ccc(NC2CCOCC2)cc1]

Compound 556 was prepared according to the procedure described in Scheme IV from 5-amino-1-(4-aminophenyl)indazole and 4-(tetrahydropyran-4-yl)aminobenzoate. [M+H]⁺ calcd for C₃₀H₂₈N₅O₃S: 538.19. Found: 538.08.

### Example 457

### N-(1-(4-Aminophenyl)-1H-indazol-5-yl)-1H-indole-6-carboxamide (Compound 557)

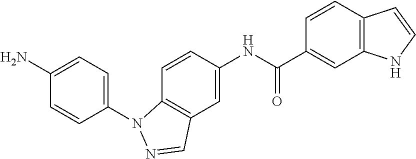 [SMILES: Nc1ccc(-n2ncc3cc(NC(=O)c4ccc5cc[nH]c5c4)ccc32)cc1]

Compound 557 was prepared according to the procedure described in Scheme IV from 5-amino-1-(4-aminophenyl)indazole and indole-6-carboxylic acid. [M+H]⁺ calcd for C₂₂H₁₇N₅O: 368.14. Found: 367.90.

### Example 458

### N-(1-(4-(Cyclopropylcarboxamido)phenyl)-1H-indazol-5-yl)-1H-indole-6-carboxamide (Compound 558)

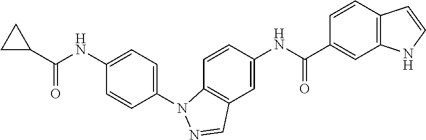 [SMILES: O=C(Nc1ccc2c(cnn2-c2ccc(NC(=O)C3CC3)cc2)c1)c1ccc2cc[nH]c2c1]

Compound 558 was prepared according to the procedure described in Scheme IV from 5-amino-1-(4-aminophenyl)indazole and indole-6-carboxylic acid. ¹H NMR (500 MHz, Acetone-d6) δ 10.64 (s, 1H), 9.65 (m, 2H), 8.79 (s, 1H), 8.54 (m, 1H), 8.21 (m, 2H), 7.91-7.67 (m, 8H), 7.54 (m, 1H), 6.58 (m, 1H), 1.82 (m, 1H), 0.94 (m, 2H), 0.82 (m, 2H).

### Example 459

### N-(4-(5-(4-(4-Hydroxypiperidin-1-yl)benzamido)-1H-indazol-1-yl)phenyl)-1-methyl-1H-indole-5-carboxamide (Compound 559)

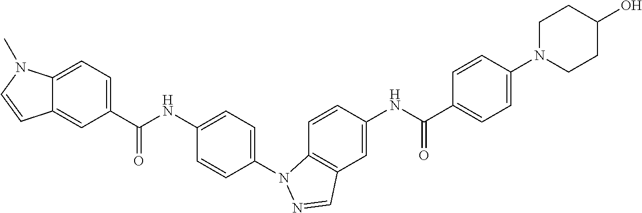 [SMILES: Cn1ccc2cc(C(=O)Nc3ccc(-n4ncc5cc(NC(=O)c6ccc(N7CCC(O)CC7)cc6)ccc54)cc3)ccc21]

Compound 559 was prepared according to the procedure described in Scheme IV from 5-amino-1-(4-aminophenyl)indazole and 4-(4-hydroxypiperidin-1-yl)benzoate. [M+H]⁺ calcd for C₃₅H₃₂N₆O₃: 585.25. Found: 585.19.

### Example 460

### N-(4-(5-(4-(4-Hydroxypiperidin-1-yl)benzamido)-1H-benzimidazol-1-yl)phenyl)cyclopropylcarboxamide (Compound 560)

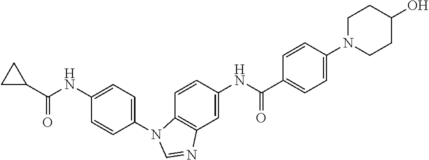 [SMILES: O=C(Nc1ccc2c(c1)ncn2-c1ccc(NC(=O)C2CC2)cc1)c1ccc(N2CCC(O)CC2)cc1]

Compound 560 was prepared according to the procedure described in Scheme IV from 5-amino-1-(4-aminophenyl)benzimidazole and 4-(4-hydroxypiperidin-1-yl)benzoate. [M+H]⁺ calcd for C₂₉H₂₉N₅O₃: 496.24. Found: 496.08.

### Example 461

### N-(4-(5-(1H-Indole-6-carboxamido)-1H-benzimidazol-1-yl)phenyl)-1H-indole-6-carboxamide (Compound 561)

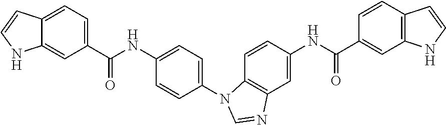 [SMILES: O=C(Nc1ccc(-n2cnc3cc(NC(=O)c4ccc5cc[nH]c5c4)ccc32)cc1)c1ccc2cc[nH]c2c1]

Compound 561 was prepared according to the procedure described in Scheme IV from 5-amino-1-(4-aminophenyl)benzimidazole and indole-6-carboxylic acid. [M+H]⁺ calcd for C₃₁H₂₂N₆O₂: 511.19. Found: 511.01.

### Example 462

### N-(1-(4-(4-Dimethylaminobenzamido)phenyl)-1H-benzimidazol-5-yl)thiophene-2-carboxamide (Compound 562)

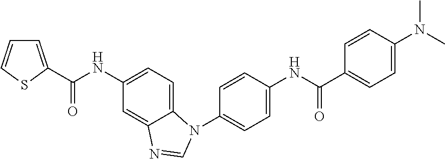 [SMILES: CN(C)c1ccc(C(=O)Nc2ccc(-n3cnc4cc(NC(=O)c5cccs5)ccc43)cc2)cc1]

Compound 562 was prepared according to the procedure described in Scheme IV from 5-amino-1-(4-aminophenyl)benzimidazole and 4-dimethylaminobenzoate. [M+H]⁺ calcd for C₂₇H₂₃N₅O₂S: 482.16. Found: 481.98.

### Example 463

### N-(4-(5-(4-Morpholinobenzamido)-1H-benzimidazol-1-yl)phenyl)cyclopropylcarboxamide (Compound 563)

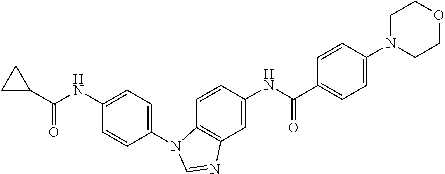 [SMILES: O=C(Nc1ccc2c(c1)ncn2-c1ccc(NC(=O)C2CC2)cc1)c1ccc(N2CCOCC2)cc1]

Compound 563 was prepared according to the procedure described in Scheme IV from 5-amino-1-(4-aminophenyl)benzimidazole and 4-morpholinobenzoate. [M+H]⁺ calcd for C₂₈H₂₇N₅O₃: 482.22. Found: 482.05.

### Example 464

### N-(4-(5-(1H-Indole-5-carboxamido)-1H-benzimidazol-1-yl)phenyl)-1H-indole-5-carboxamide (Compound 564)

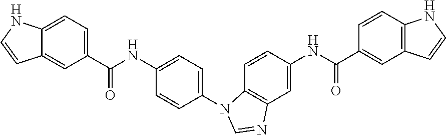 [SMILES: O=C(Nc1ccc(-n2cnc3cc(NC(=O)c4ccc5[nH]ccc5c4)ccc32)cc1)c1ccc2[nH]ccc2c1]

Compound 564 was prepared according to the procedure described in Scheme IV from 5-amino-1-(4-aminophenyl)benzimidazole and indole-5-carboxylic acid. [M+H]⁺ calcd for C₃₁H₂₂N₆O₂: 511.19. Found: 511.07.

### Example 465

### N,N′-(4,4′-Azanediylbis(4,1-phenylene))bis(4-(1H-imidazol-1-yl)benzamide) (Compound 565)

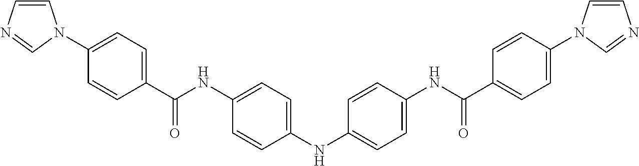 [SMILES: O=C(Nc1ccc(Nc2ccc(NC(=O)c3ccc(-n4ccnc4)cc3)cc2)cc1)c1ccc(-n2ccnc2)cc1]

Compound 565 was prepared according to the procedure described in Scheme IV from 4,4′-diaminodiphenylamine and 4-(imidazol-1-yl)benzoate. [M+H]⁺ calcd for C₃₂H₂₅N₇O₂: 540.21. Found 539.97.

### Example 466

### N,N′-(4,4′-Oxabis(4,1-phenylene))bis(4-(1H-imidazol-1-yl)benzamide) (Compound 566)

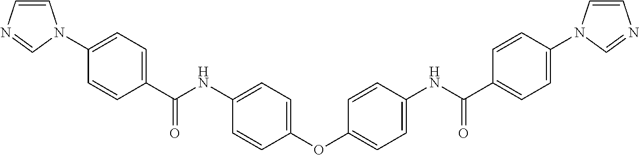 [SMILES: O=C(Nc1ccc(Oc2ccc(NC(=O)c3ccc(-n4ccnc4)cc3)cc2)cc1)c1ccc(-n2ccnc2)cc1]

Compound 566 was prepared according to the procedure described in Scheme IV from 4,4′-oxabisphenylamine and 4-(imidazol-1-yl)benzoate. [M+H]⁺ calcd for C₃₂H₂₄N₆O₃: 541.20. Found 541.05.

### Example 467

### N,N′-(4,4′-Azanediylbis(4,1-phenylene))bis(4-(1H-pyrazol-1-yl)benzamide) (Compound 567)

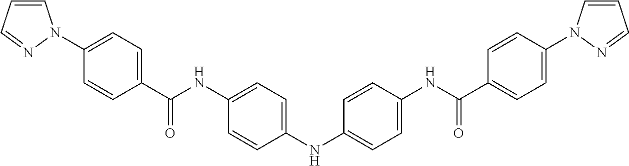 [SMILES: O=C(Nc1ccc(Nc2ccc(NC(=O)c3ccc(-n4cccn4)cc3)cc2)cc1)c1ccc(-n2cccn2)cc1]

Compound 567 was prepared according to the procedure described in Scheme IV from 4,4′-diaminodiphenylamine and 4-(pyrazol-1-yl)benzoate. [M+H]calcd for C₃₂H₂₅N₇O₂: 540.21. Found 539.97.

### Example 468

### N,N′-(4,4′-Azanediylbis(4,1-phenylene))bis(4-(1H-pyrrol-1-yl)benzamide) (Compound 568)

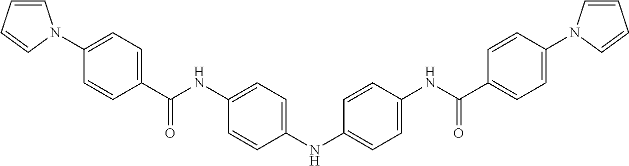 [SMILES: O=C(Nc1ccc(Nc2ccc(NC(=O)c3ccc(-n4cccc4)cc3)cc2)cc1)c1ccc(-n2cccc2)cc1]

Compound 568 was prepared according to the procedure described in Scheme IV from 4,4′-diaminodiphenylamine and 4-(pyrrol-1-yl)benzoate. [M+H]⁺ calcd for C₃₄H₂₇N₅O₂: 538.22. Found 538.09.

### Example 469

### N,N′-(4,4′-Azanediylbis(4,1-phenylene))bis(4-hydroxybenzamide) (Compound 569)

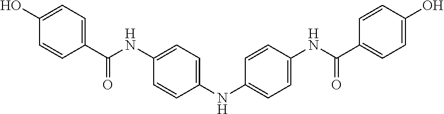 [SMILES: O=C(Nc1ccc(Nc2ccc(NC(=O)c3ccc(O)cc3)cc2)cc1)c1ccc(O)cc1]

Compound 569 was prepared according to the procedure described in Scheme IV from 4,4′-diaminodiphenylamine and 4-hydroxybenzoate. [M+H]⁺ calcd for C₂₆H₂₁N₃O₄: 440.15. Found 439.94.

### Example 470

### N,N′-(4,4′-Azanediylbis(4,1-phenylene))bis(3-hydroxybenzamide) (Compound 569)

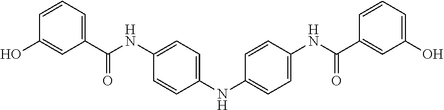 [SMILES: O=C(Nc1ccc(Nc2ccc(NC(=O)c3cccc(O)c3)cc2)cc1)c1cccc(O)c1]

Compound 570 was prepared according to the procedure described in Scheme IV from 4,4′-diaminodiphenylamine and 3-hydroxybenzoate. [M+H]⁺ calcd for C₂₆H₂₁N₃O₄: 440.15. Found 493.94.

### Example 471

### N,N′-(4,4′-Azanediylbis(4,1-phenylene))bis(4-methoxybenzamide) (Compound 571)

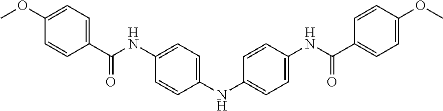

Compound 571 was prepared according to the procedure described in Scheme IV from 4,4′-diaminodiphenylamine and 4-methoxybenzoate. [M+H]⁺ calcd for C₂₈H₂₅N₃O₄: 468.18. Found 468.01.

### Example 472

### N,N′-(4,4′-Azanediylbis(4,1-phenylene))bis(1H-indole-2-carboxamide) (Compound 572)

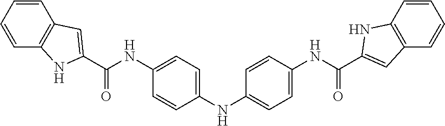 [SMILES: O=C(Nc1ccc(Nc2ccc(NC(=O)c3cc4ccccc4[nH]3)cc2)cc1)c1cc2ccccc2[nH]1]

Compound 572 was prepared according to the procedure described in Scheme IV from 4,4′-diaminodiphenylamine and indole-2-carboxylic acid. [M+H]⁺ calcd for C₃₀H₂₃N₅O₂: 486.19. Found 486.10.

### Example 473

### N,N′-(4,4′-Azanediylbis(4,1-phenylene))bis(4-(4-hydroxyphenyl)benzamide) (Compound 573)

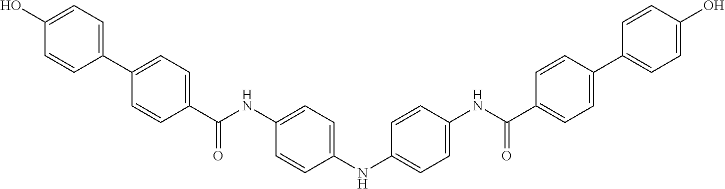 [SMILES: O=C(Nc1ccc(Nc2ccc(NC(=O)c3ccc(-c4ccc(O)cc4)cc3)cc2)cc1)c1ccc(-c2ccc(O)cc2)cc1]

Compound 573 was prepared according to the procedure described in Scheme IV from 4,4′-diaminodiphenylamine and 4-(4-hydroxyphenyl)benzoate. [M+H]⁺ calcd for C₃₈H₂₉N₃O₄: 592.22. Found 592.12.

### Example 474

### N,N′-(4,4′-Oxabis(4,1-phenylene))bis(4-aminobenzamide) (Compound 574)

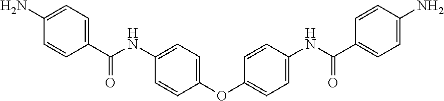 [SMILES: Nc1ccc(C(=O)Nc2ccc(Oc3ccc(NC(=O)c4ccc(N)cc4)cc3)cc2)cc1]

Compound 574 was prepared according to the procedure described in Scheme IV from 4,4′-oxabisphenylamine and 4-aminobenzoate. [M+H]⁺ calcd for C₂₆H₂₂N₄O₃: 439.17. Found 439.00.

### Example 475

### N,N′-(4,4′-Azanediylbis(4,1-phenylene))bis(1H-indole-6-carboxamide) (Compound 575)

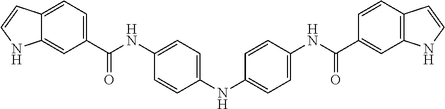 [SMILES: O=C(Nc1ccc(Nc2ccc(NC(=O)c3ccc4cc[nH]c4c3)cc2)cc1)c1ccc2cc[nH]c2c1]

Compound 575 was prepared according to the procedure described in Scheme IV from 4,4′-diaminodiphenylamine and indole-6-carboxylic acid. [M+H]⁺ calcd for C₃₀H₂₃N₅O₂: 486.19. Found 486.03.

### Example 476

### N,N′-(4,4′-Azanediylbis(4,1-phenylene))bis(1H-indole-5-carboxamide) (Compound 576)

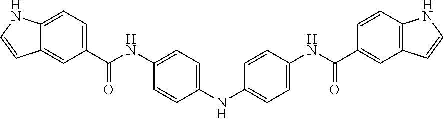 [SMILES: O=C(Nc1ccc(Nc2ccc(NC(=O)c3ccc4[nH]ccc4c3)cc2)cc1)c1ccc2[nH]ccc2c1]

Compound 576 was prepared according to the procedure described in Scheme IV from 4,4′-diaminodiphenylamine and indole-5-carboxylic acid. [M+H]⁺ calcd for C₃₀H₂₃N₅O₂: 486.19. Found 486.03.

### Example 477

### N,N′-(4,4′-Azanediylbis(4,1-phenylene))bis(4-(2-hydroxyethylamino)benzamide) (Compound 577)

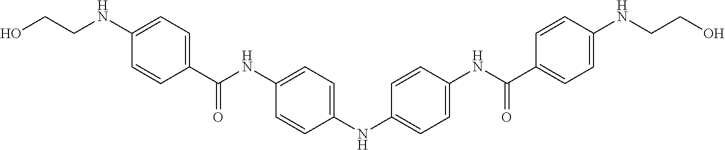 [SMILES: O=C(Nc1ccc(Nc2ccc(NC(=O)c3ccc(NCCO)cc3)cc2)cc1)c1ccc(NCCO)cc1]

Compound 577 was prepared according to the procedure described in Scheme IV from 4,4′-diaminodiphenylamine and 4-(2-hydroxyethylamino)benzoate. [M+H]⁺ calcd for C₃₀H₃₂N₅O₄: 526.25. Found: 526.08.

### Example 478

### N,N′-(4,4′-Azanediylbis(4,1-phenylene))bis(4-methanesulfonamidobenzamide) (Compound 578)

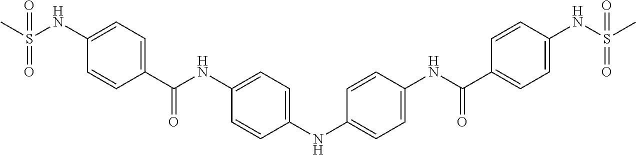

Compound 578 was prepared according to the procedure described in Scheme IV from 4,4′-diaminodiphenylamine and 4-methanesulfonamidobenzoate. [M+H]⁺ calcd for C₂₈H₂₇N₅O₆S₂: 594.14. Found 594.00.

### Example 479

### N,N′-(4,4′-Azanediylbis(4,1-phenylene))bis(4-tert-butyloxycarboxamidobenzamide) (Compound 579)

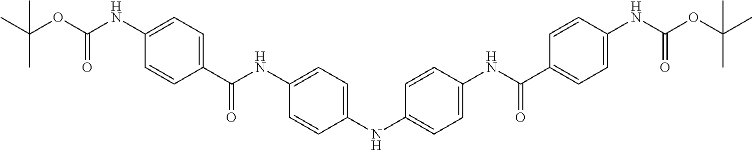 [SMILES: CC(C)(C)OC(=O)Nc1ccc(C(=O)Nc2ccc(Nc3ccc(NC(=O)c4ccc(NC(=O)OC(C)(C)C)cc4)cc3)cc2)cc1]

Compound 579 was prepared according to the procedure described in Scheme IV from 4,4′-diaminodiphenylamine and 4-tert-butyloxycarboxamidobenzoate. [M+H]⁺ calcd for C₃₆H₃₉N₅O₆: 638.29. Found 638.20.

### Example 480

### N,N′-(4,4′-Azanediylbis(4,1-phenylene))bis(4-aminobenzamide) (Compound 580)

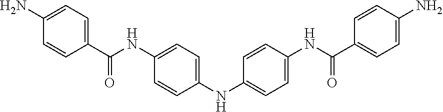 [SMILES: Nc1ccc(C(=O)Nc2ccc(Nc3ccc(NC(=O)c4ccc(N)cc4)cc3)cc2)cc1]

Compound 580 was prepared according to the procedure described in Scheme IV from 4,4′-diaminodiphenylamine and 4-aminobenzoate. [M+H]⁺ calcd for C₂₆H₂₃N₅O₂: 438.19. Found 438.05.

### Example 481

### N,N′-(4,4′-Azanediylbis(4,1-phenylene))bis(4-(2-aminoacetamidobenzamide) (Compound 581)

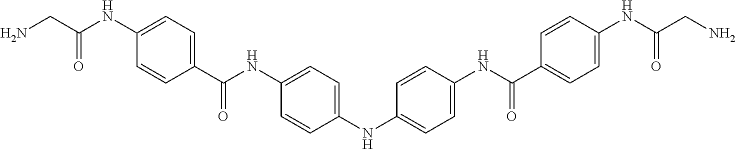 [SMILES: NCC(=O)Nc1ccc(C(=O)Nc2ccc(Nc3ccc(NC(=O)c4ccc(NC(=O)CN)cc4)cc3)cc2)cc1]

Compound 581 was prepared according to the procedure described in Scheme IV from 4,4′-diaminodiphenylamine and 4-(2-aminoacetamido)benzoate. [M+H]⁺ calcd for C₃₀H₂₉N₇O₄: 552.23. Found 552.12.

### Example 482

### N,N′-(4,4′-Azanediylbis(4,1-phenylene))bis(1-methyl-1H-indole-5-carboxamide) (Compound 582)

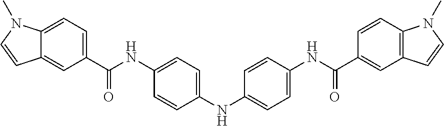 [SMILES: Cn1ccc2cc(C(=O)Nc3ccc(Nc4ccc(NC(=O)c5ccc6c(ccn6C)c5)cc4)cc3)ccc21]

Compound 582 was prepared according to the procedure described in Scheme IV from 4,4′-diaminodiphenylamine and 1-methylindole-5-carboxylic acid. [M+H]⁺ calcd for C₃₂H₂₇N₅O₂: 514.22. Found 514.11.

### Example 483

### N,N′-(4,4′-Azanediylbis(4,1-phenylene))bis(thiophene-2-carboxamide) (Compound 583)

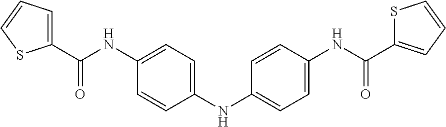 [SMILES: O=C(Nc1ccc(Nc2ccc(NC(=O)c3cccs3)cc2)cc1)c1cccs1]

Compound 583 was prepared according to the procedure described in Scheme IV from 4,4′-diaminodiphenylamine and thiophene-2-carboxylic acid. [M+H]⁺ calcd for C₂₂H₁₇N₃O₂S₂: 420.08. Found 419.91.

### Example 484

### N,N′-(4,4′-Azanediylbis(4,1-phenylene))bis(4-pyrrolidinebenzamide) (Compound 584)

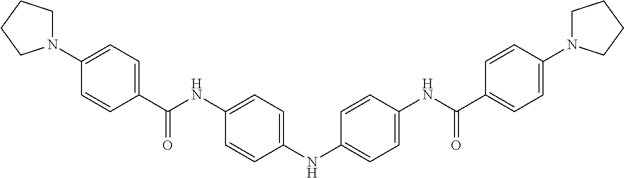 [SMILES: O=C(Nc1ccc(Nc2ccc(NC(=O)c3ccc(N4CCCC4)cc3)cc2)cc1)c1ccc(N2CCCC2)cc1]

Compound 584 was prepared according to the procedure described in Scheme IV from 4,4′-diaminodiphenylamine and 4-pyrrolidinebenzoate. [M+H]⁺ calcd for C₃₄H₃₅N₅O₂: 546.28. Found 546.07.

### Example 485

### N,N′-(4,4′-Oxabis(4,1-phenylene))bis(1H-indole-5-carboxamide) (Compound 585)

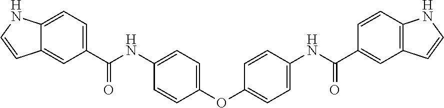 [SMILES: O=C(Nc1ccc(Oc2ccc(NC(=O)c3ccc4[nH]ccc4c3)cc2)cc1)c1ccc2[nH]ccc2c1]

Compound 585 was prepared according to the procedure described in Scheme IV from 4,4′-diaminodiphenylamine and indole-5-carboxylic acid. [M+H]⁺ calcd for C₃₀H₂₂N₄O₃: 487.17. Found 486.98.

### Example 486

### N,N′-(4,4′-Oxabis(4,1-phenylene))bis(4-N-methyl-N-(2-hydroxyethyl)aminobenzamide) (Compound 586)

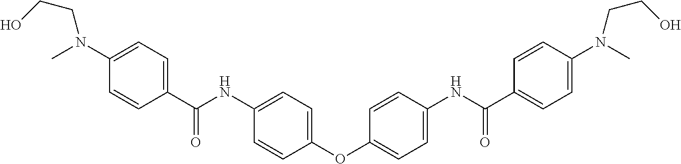 [SMILES: CN(CCO)c1ccc(C(=O)Nc2ccc(Oc3ccc(NC(=O)c4ccc(N(C)CCO)cc4)cc3)cc2)cc1]

Compound 586 was prepared according to the procedure described in Scheme IV from 4,4′-diaminodiphenylamine and 4-N-methyl-N-(2-hydroxyethyl)aminobenzoate. [M+H]⁺ calcd for C₃₂H₃₄N₄O₅: 555.25. Found 555.12.

### Example 487

### N,N′-(4,4′-Azanediylbis(4,1-phenylene))bis(1-methyl-1,4-benzoxazine-6-carboxamide) (Compound 587)

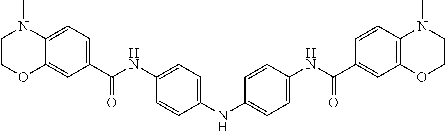 [SMILES: CN1CCOc2cc(C(=O)Nc3ccc(Nc4ccc(NC(=O)c5ccc6c(c5)OCCN6C)cc4)cc3)ccc21]

Compound 587 was prepared according to the procedure described in Scheme IV from 4,4′-diaminodiphenylamine and 1-methyl-1,4-benzoxazine-6-carboxylic acid. [M+H]⁺ calcd for C₃₂H₃₁N₅O₄: 550.24. Found 550.05.

### Example 488

### N,N′-(4,4′-Carbonylbis(4,1-phenylene))bis(4-aminobenzamide) (Compound 588)

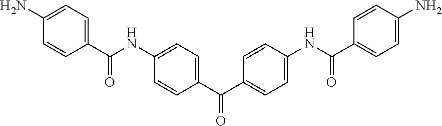 [SMILES: Nc1ccc(C(=O)Nc2ccc(C(=O)c3ccc(NC(=O)c4ccc(N)cc4)cc3)cc2)cc1]

Compound 588 was prepared according to the procedure described in Scheme IV from 4,4′-diaminobenzophenone and 4-aminobenzoate. [M+H]⁺ calcd for C₂₇H₂₂N₄O₃: 451.17. Found 450.94.

### Example 489

### N,N′-(4,4′-Carbonylbis(4,1-phenylene))bis(4-tert-butyloxycarbamidobenzamide) (Compound 589)

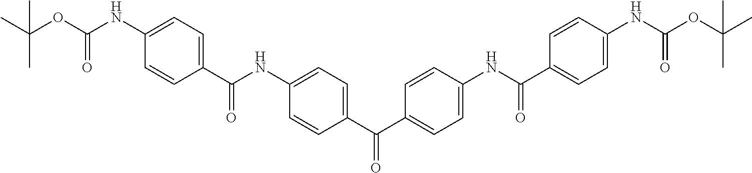 [SMILES: CC(C)(C)OC(=O)Nc1ccc(C(=O)Nc2ccc(C(=O)c3ccc(NC(=O)c4ccc(NC(=O)OC(C)(C)C)cc4)cc3)cc2)cc1]

Compound 589 was prepared according to the procedure described in Scheme IV from 4,4′-diaminobenzophenone and 4-tert-butyloxycarbamidobenzoate. [M+H]⁺ calcd for C₃₇H₃₈N₄O₇: 651.27. Found 651.10.

### Example 490

### N,N′-(4,4′-Azanediylbis(4,1-phenylene))bis(1H-benzimidazole-5-carboxamide) (Compound 590)

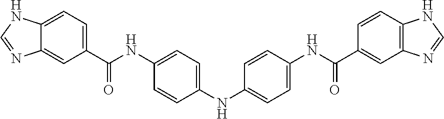 [SMILES: O=C(Nc1ccc(Nc2ccc(NC(=O)c3ccc4[nH]cnc4c3)cc2)cc1)c1ccc2[nH]cnc2c1]

Compound 590 was prepared according to the procedure described in Scheme IV from 4,4′-diaminodiphenylamine and benzimidazole-5-carboxylic acid. [M+H]⁺ calcd for C₂₈H₂₁N₇O₂: 488.18. Found 488.02.

### Example 491

### 4-(4-Cyclopropylcarbonyl)piperazin-1-yl-N-(4-(4-ethylaminobenzoyl)phenyl)benzamide (Compound 591)

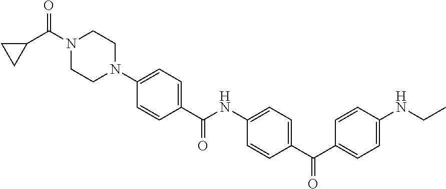 [SMILES: CCNc1ccc(C(=O)c2ccc(NC(=O)c3ccc(N4CCN(C(=O)C5CC5)CC4)cc3)cc2)cc1]

Compound 591 was prepared according to the procedure described in Scheme IV from 4,4′-diaminobenzophenone and (4-(4-cyclopropylcarbonyl)piperazin-1-yl)benzoate. [M+H]⁺ calcd for C₃₀H₃₂N₄O₃: 497.12. Found: 497.33.

### Example 492

### 4-Morpholino-N-(4-(4-diethylaminobenzoyl)phenyl)benzamide (Compound 592)

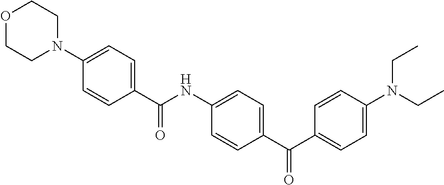 [SMILES: CCN(CC)c1ccc(C(=O)c2ccc(NC(=O)c3ccc(N4CCOCC4)cc3)cc2)cc1]

Compound 592 was prepared according to the procedure described in Scheme IV from 4,4′-diaminobenzophenone and 4-morpholinobenzoate. [M+H]⁺ calcd for C₂₈H₃₁N₃O₃: 458.09. Found: 458.31.

### Example 493

### 4-Morpholino-N-(4-(4-propylaminobenzoyl)phenyl)benzamide (Compound 593)

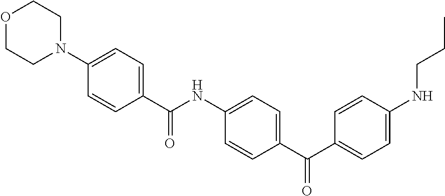 [SMILES: CCCNc1ccc(C(=O)c2ccc(NC(=O)c3ccc(N4CCOCC4)cc3)cc2)cc1]

Compound 593 was prepared according to the procedure described in Scheme IV from 4,4′-diaminobenzophenone and 4-morpholinobenzoate. [M+H]⁺ calcd for C₂₇H₂₉N₃O₃: 444.06. Found: 444.31.

### Example 494

### 4-Morpholino-N-(4-(4-allylaminobenzoyl)phenyl)benzamide (Compound 594)

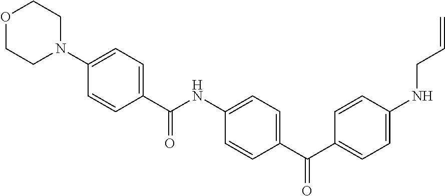 [SMILES: C=CCNc1ccc(C(=O)c2ccc(NC(=O)c3ccc(N4CCOCC4)cc3)cc2)cc1]

Compound 594 was prepared according to the procedure described in Scheme IV from 4,4′-diaminobenzophenone and 4-morpholinobenzoate. [M+H]⁺ calcd for C₂₇H₂₇N₃O₃: 442.04. Found: 442.30.

### Example 495

### 4-Morpholino-N-(4-(4-(furan-2-carboxamido)benzoyl)phenyl)benzamide (Compound 595)

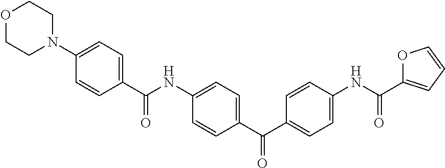 [SMILES: O=C(Nc1ccc(C(=O)c2ccc(NC(=O)c3ccco3)cc2)cc1)c1ccc(N2CCOCC2)cc1]

Compound 595 was prepared according to the procedure described in Scheme IV from 4,4′-diaminobenzophenone and 4-morpholinobenzoate. [M+H]⁺ calcd for C₂₉H₂₅N₃O₅: 496.05. Found: 496.09.

### Example 496

### N-(1-(4-Acetylpheyl)-1H-indazol-5-yl)morpholinobenzamide (Compound 596)

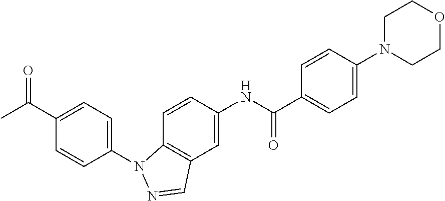 [SMILES: CC(=O)c1ccc(-n2ncc3cc(NC(=O)c4ccc(N5CCOCC5)cc4)ccc32)cc1]

Compound 596 was prepared according to the procedure described in Scheme IV from 5-amino-(4-acetylphenyl)indazole and 4-morpholinobenzoate. [M+H]⁺ calcd for C₂₆H₂₄N₄O₃: 441.18. Found: 441.02.

### Example 497

### 4-Morpholino-N-(4-(4-(pyridine-4-carboxamido)benzoyl)phenyl)benzamide (Compound 597)

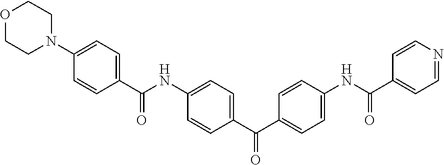 [SMILES: O=C(Nc1ccc(C(=O)c2ccc(NC(=O)c3ccc(N4CCOCC4)cc3)cc2)cc1)c1ccncc1]

Compound 597 was prepared according to the procedure described in Scheme IV from 4,4′-diaminobenzophenone and 4-morpholinobenzoate. [M+H]⁺ calcd for C₃₀H₂₇N₄O₄: 507.20. Found: 507.02.

### Example 498

### 4-Morpholino-N-(4-(4-(pyridine-3-carboxamido)benzoyl)phenyl)benzamide (Compound 598)

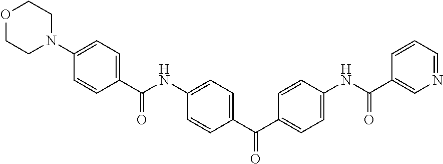 [SMILES: O=C(Nc1ccc(C(=O)c2ccc(NC(=O)c3cccnc3)cc2)cc1)c1ccc(N2CCOCC2)cc1]

Compound 598 was prepared according to the procedure described in Scheme IV from 4,4′-diaminobenzophenone and 4-morpholinobenzoate. [M+H]⁺ calcd for C₃₀H₂₆N₄O₄: 507.08. Found: 507.09.

### Example 499

### 4-Morpholino-N-(4-(4-(2-methoxycarbonylethyl)aminobenzoyl)phenyl)benzamide (Compound 599)

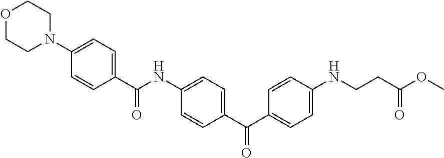 [SMILES: COC(=O)CCNc1ccc(C(=O)c2ccc(NC(=O)c3ccc(N4CCOCC4)cc3)cc2)cc1]

Compound 599 was prepared according to the procedure described in Scheme IV from 4,4′-diaminobenzophenone and 4-morpholinobenzoate. [M+H]⁺ calcd for C₂₈H₂₉N₃O₅: 488.07. Found: 488.08.

### Example 500

### 4-Morpholino-N-(4-(4-(isoxazole-5-carboxamido)benzoyl)phenyl)benzamide (Compound 600)

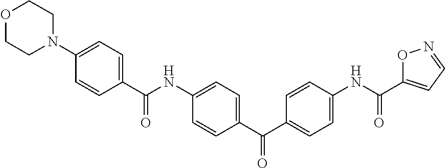 [SMILES: O=C(Nc1ccc(C(=O)c2ccc(NC(=O)c3ccno3)cc2)cc1)c1ccc(N2CCOCC2)cc1]

Compound 600 was prepared according to the procedure described in Scheme IV from 4,4′-diaminobenzophenone and 4-morpholinobenzoate. [M+H]⁺ calcd for C₂₈H₂₄N₄O₅: 497.04. Found: 497.03.

### Example 501

### 4-(4-Cyclopropylcarbonyl)piperazin-1-yl-N-(4-(4-aminobenzoyl)phenyl)benzamide (Compound 601)

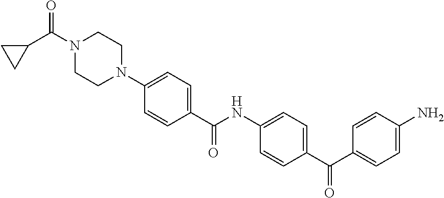 [SMILES: Nc1ccc(C(=O)c2ccc(NC(=O)c3ccc(N4CCN(C(=O)C5CC5)CC4)cc3)cc2)cc1]

Compound 601 was prepared according to the procedure described in Scheme IV from 4,4′-diaminobenzophenone and (4-(4-cyclopropylcarbonyl)piperazin-1-yl)benzoate. [M+H]⁺ calcd for C₂₈H₂₈N₄O₃: 469.07. Found: 469.09.

### Example 502

### 4-Morpholino-N-(4-(4-(1,2,4-triazole-3-carboxamido)benzoyl)phenyl)benzamide (Compound 602)

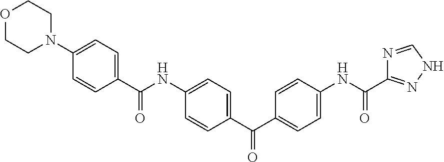 [SMILES: O=C(Nc1ccc(C(=O)c2ccc(NC(=O)c3nc[nH]n3)cc2)cc1)c1ccc(N2CCOCC2)cc1]

Compound 602 was prepared according to the procedure described in Scheme IV from 4,4′-diaminobenzophenone and 4-morpholinobenzoate. [M+H]⁺ calcd for C₂₇H₂₄N₆O₄: 497.04. Found: 497.03.

### Example 503

### 4-Morpholino-N-(4-(4-(pyridazine-4-carboxamido)benzoyl)phenyl)benzamide (Compound 603)

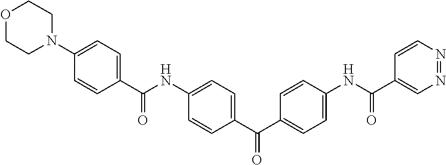 [SMILES: O=C(Nc1ccc(C(=O)c2ccc(NC(=O)c3ccnnc3)cc2)cc1)c1ccc(N2CCOCC2)cc1]

Compound 603 was prepared according to the procedure described in Scheme IV from 4,4′-diaminobenzophenone and 4-morpholinobenzoate. [M+H]⁺ calcd for C₂₉H₂₅N₅O₄: 508.06. Found: 508.04.

### Example 504

### 4-Morpholino-N-(4-(4-(3-cyclopropylpyrazole-5-carboxamido)benzoyl)phenyl)benzamide (Compound 604)

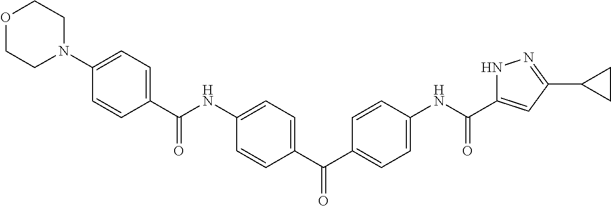 [SMILES: O=C(Nc1ccc(C(=O)c2ccc(NC(=O)c3cc(C4CC4)n[nH]3)cc2)cc1)c1ccc(N2CCOCC2)cc1]

Compound 604 was prepared according to the procedure described in Scheme IV from 4,4′-diaminobenzophenone and 4-morpholinobenzoate. [M+H]⁺ calcd for C₃₁H₃₀N₅O₄: 536.23. Found: 536.10.

### Example 505

### 4-Morpholino-N-(4-(4-(1,2,3-thiodiazol-4-carboxamido)benzoyl)phenyl)benzamide (Compound 605)

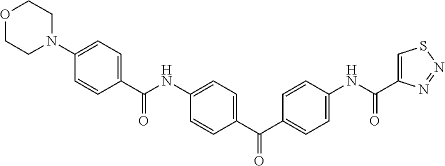 [SMILES: O=C(Nc1ccc(C(=O)c2ccc(NC(=O)c3csnn3)cc2)cc1)c1ccc(N2CCOCC2)cc1]

Compound 605 was prepared according to the procedure described in Scheme IV from 4,4′-diaminobenzophenone and 4-morpholinobenzoate. [M+H]⁺ calcd for C₂₇H₂₃N₅O₄S: 514.09. Found: 514.04.

### Example 506

### 4-Morpholino-N-(4-(4-(thiazole-4-carboxamido)benzoyl)phenyl)benzamide (Compound 606)

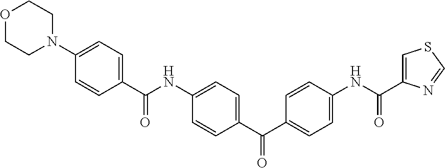 [SMILES: O=C(Nc1ccc(C(=O)c2ccc(NC(=O)c3cscn3)cc2)cc1)c1ccc(N2CCOCC2)cc1]

Compound 606 was prepared according to the procedure described in Scheme IV from 4,4′-diaminobenzophenone and 4-morpholinobenzoate. [M+H]⁺ calcd for C₂₈H₂₄N₄O₄S: 513.10. Found: 513.03.

### Example 507

### 4-Morpholino-N-(4-(4-(imidazole-4-carboxamido)benzoyl)phenyl)benzamide (Compound 607)

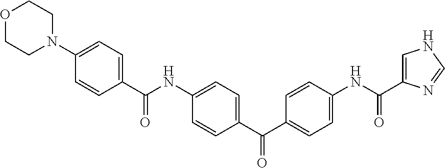 [SMILES: O=C(Nc1ccc(C(=O)c2ccc(NC(=O)c3c[nH]cn3)cc2)cc1)c1ccc(N2CCOCC2)cc1]

Compound 607 was prepared according to the procedure described in Scheme IV from 4,4′-diaminobenzophenone and 4-morpholinobenzoate. [M+H]⁺ calcd for C₂₈H₂₆N₅O₄: 496.20. Found: 496.02.

### Example 508

### 4-Morpholino-N-(4-(4-(5-methylisoxazole-3-carboxamido)benzoyl)phenyl)benzamide (Compound 608)

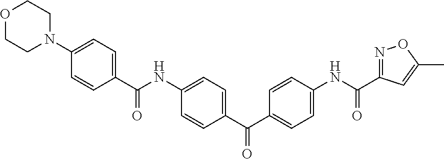 [SMILES: Cc1cc(C(=O)Nc2ccc(C(=O)c3ccc(NC(=O)c4ccc(N5CCOCC5)cc4)cc3)cc2)no1]

Compound 608 was prepared according to the procedure described in Scheme IV from 4,4′-diaminobenzophenone and 4-morpholinobenzoate. [M+H]⁺ calcd for C₂₉H₂₆N₄O₅: 511.06. Found: 511.07.

### Example 509

### 4-Morpholino-N-(4-(4-(oxazole-4-carboxamido)benzoyl)phenyl)benzamide (Compound 609)

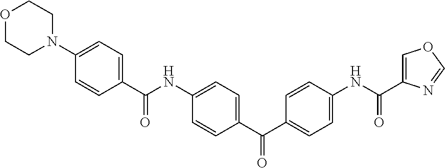 [SMILES: O=C(Nc1ccc(C(=O)c2ccc(NC(=O)c3cocn3)cc2)cc1)c1ccc(N2CCOCC2)cc1]

Compound 609 was prepared according to the procedure described in Scheme IV from 4,4′-diaminobenzophenone and 4-morpholinobenzoate. [M+H]⁺ calcd for C₂₈H₂₄N₄O₅: 497.04. Found: 497.03.

### Example 510

### 4-Morpholino-N-(4-(4-(1,2,3-triazole-4-carboxamido)benzoyl)phenyl)benzamide (Compound 610)

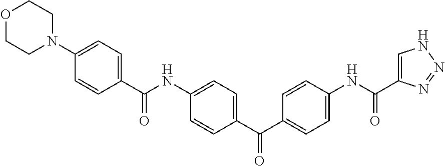 [SMILES: O=C(Nc1ccc(C(=O)c2ccc(NC(=O)c3c[nH]nn3)cc2)cc1)c1ccc(N2CCOCC2)cc1]

Compound 610 was prepared according to the procedure described in Scheme IV from 4,4′-diaminobenzophenone and 4-morpholinobenzoate. [M+H]⁺ calcd for C₂₇H₂₄N₆O₄: 497.04. Found: 497.03.

### Example 511

### 4-Morpholino-N-(4-(4-(pyridine-2-carboxamido)benzoyl)phenyl)benzamide (Compound 611)

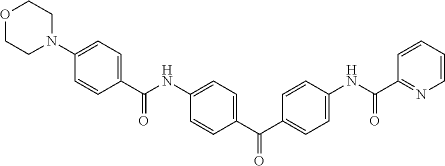 [SMILES: O=C(Nc1ccc(C(=O)c2ccc(NC(=O)c3ccccn3)cc2)cc1)c1ccc(N2CCOCC2)cc1]

Compound 611 was prepared according to the procedure described in Scheme IV from 4,4′-diaminobenzophenone and 4-morpholinobenzoate. [M+H]⁺ calcd for C₃₀H₂₆N₄O₄: 507.08. Found: 507.02.

### Example 512

### 4-Morpholino-N-(4-(4-(pyridazine-3-carboxamido)benzoyl)phenyl)benzamide (Compound 612)

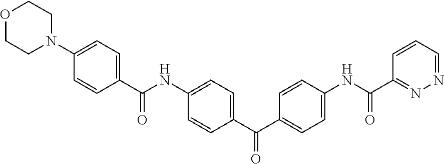 [SMILES: O=C(Nc1ccc(C(=O)c2ccc(NC(=O)c3cccnn3)cc2)cc1)c1ccc(N2CCOCC2)cc1]

Compound 612 was prepared according to the procedure described in Scheme IV from 4,4′-diaminobenzophenone and 4-morpholinobenzoate. [M+H]⁺ calcd for C₂₉H₂₅N₅O₄: 508.06. Found: 508.29.

### Example 513

### 4-Morpholino-N-(4-(4-(1-methyl-1H-pyrazole-3-carboxamido)benzoyl)phenyl)benzamide (Compound 613)

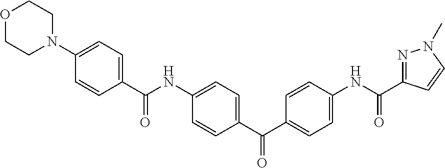 [SMILES: Cn1ccc(C(=O)Nc2ccc(C(=O)c3ccc(NC(=O)c4ccc(N5CCOCC5)cc4)cc3)cc2)n1]

Compound 613 was prepared according to the procedure described in Scheme IV from 4,4′-diaminobenzophenone and 4-morpholinobenzoate. [M+H]⁺ calcd for C₂₉H₂₇N₅O₄: 510.08. Found: 510.06.

### Example 514

### 4-Morpholino-N-(4-(4-(pyrazine-2-carboxamido)benzoyl)phenyl)benzamide (Compound 614)

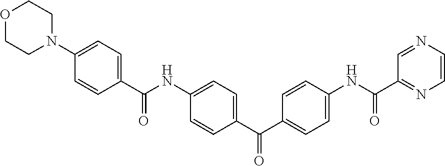 [SMILES: O=C(Nc1ccc(C(=O)c2ccc(NC(=O)c3cnccn3)cc2)cc1)c1ccc(N2CCOCC2)cc1]

Compound 614 was prepared according to the procedure described in Scheme IV from 4,4′-diaminobenzophenone and 4-morpholinobenzoate. [M+H]⁺ calcd for C₂₉H₂₅N₅O₄: 508.06. Found: 508.04.

### Example 515

### 4-Morpholino-N-(4-(4-(thiophene-2-carbanylcarboxamido)benzoyl)phenyl)benzamide (Compound 615)

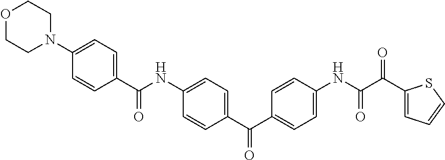 [SMILES: O=C(Nc1ccc(C(=O)c2ccc(NC(=O)c3ccc(N4CCOCC4)cc3)cc2)cc1)C(=O)c1cccs1]

Compound 615 was prepared according to the procedure described in Scheme IV from 4,4′-diaminobenzophenone and 4-morpholinobenzoate. [M+H]⁺ calcd for C₃₀H₂₅N₃O₅S: 540.12. Found: 540.03.

### Example 516

### 4-Morpholino-N-(4-(4-acetylcarboxamido)benzoyl)phenyl)benzamide (Compound 616)

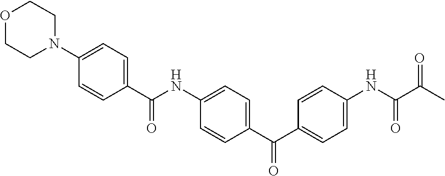 [SMILES: CC(=O)C(=O)Nc1ccc(C(=O)c2ccc(NC(=O)c3ccc(N4CCOCC4)cc3)cc2)cc1]

Compound 616 was prepared according to the procedure described in Scheme IV from 4,4′-diaminobenzophenone and 4-morpholinobenzoate. [M+H]⁺ calcd for C₂₇H₂₅N₃O₅: 472.03. Found: 472.06.

### Example 517

### 3-Chloro-4-morpholino-N-(4-(4-aminobenzoyl)phenyl)benzamide (Compound 617)

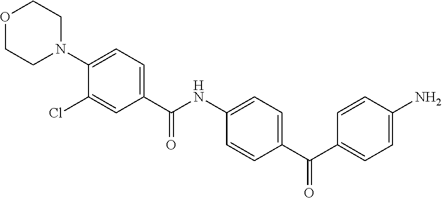 [SMILES: Nc1ccc(C(=O)c2ccc(NC(=O)c3ccc(N4CCOCC4)c(Cl)c3)cc2)cc1]

Compound 617 was prepared according to the procedure described in Scheme IV from 4,4′-diaminobenzophenone and 3-chloro-4-morpholinobenzoate.

[M+H]⁺ calcd for C₂₄H₂₃ClN₃O₃: 436.14. Found: 435.97.

### Example 518

### 3-Chloro-4-morpholino-N-(4-(4-(thiophene-2-carboxamido)benzoyl)phenyl)benzamide (Compound 618)

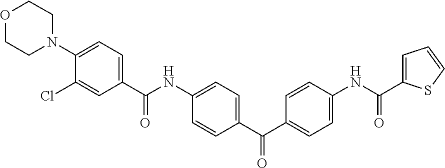 [SMILES: O=C(Nc1ccc(C(=O)c2ccc(NC(=O)c3cccs3)cc2)cc1)c1ccc(N2CCOCC2)c(Cl)c1]

Compound 618 was prepared according to the procedure described in Scheme IV from 4,4′-diaminobenzophenone and 3-chloro-4-morpholinobenzoate. [M+H]⁺ calcd for C₂₉H₂₅ClN₃O₄S: 546.13. Found: 546.08.

### Example 519

### 4-Morpholino-N-(4-(4-(2,6-dichloropyridine-4-carboxamido)benzoyl)phenyl)benzamide (Compound 619)

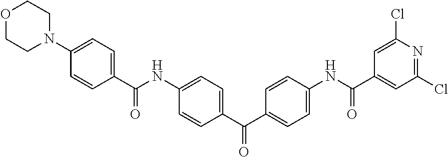 [SMILES: O=C(Nc1ccc(C(=O)c2ccc(NC(=O)c3cc(Cl)nc(Cl)c3)cc2)cc1)c1ccc(N2CCOCC2)cc1]

Compound 619 was prepared according to the procedure described in Scheme IV from 4,4′-diaminobenzophenone and 4-morpholinobenzoate. [M+H]⁺ calcd for C₃₀H₂₄Cl₂N₄O₄: 576.07. Found: 576.15.

### Example 520

### 4-Morpholino-N-(4-(4-(2-chloro-6-methylpyridine-4-carboxamido)benzoyl)phenyl)benzamide (Compound 620)

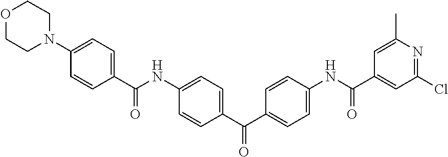 [SMILES: Cc1cc(C(=O)Nc2ccc(C(=O)c3ccc(NC(=O)c4ccc(N5CCOCC5)cc4)cc3)cc2)cc(Cl)n1]

Compound 620 was prepared according to the procedure described in Scheme IV from 4,4′-diaminobenzophenone and 4-morpholinobenzoate. [M+H]⁺ calcd for C₃₁H₂₇ClN₄O₄: 556.05. Found: 556.17.

### Example 521

### 4-Morpholino-N-(4-(4-(2-chloropyridine-4-carboxamido)benzoyl)phenyl)benzamide (Compound 621)

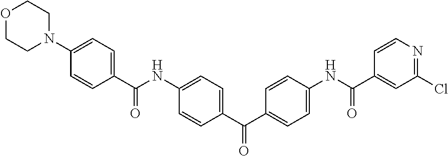 [SMILES: O=C(Nc1ccc(C(=O)c2ccc(NC(=O)c3ccnc(Cl)c3)cc2)cc1)c1ccc(N2CCOCC2)cc1]

Compound 621 was prepared according to the procedure described in Scheme IV from 4,4′-diaminobenzophenone and 4-morpholinobenzoate. [M+H]⁺ calcd for C₃₀H₂₅ClN₄O₄: 542.02. Found: 541.92.

### Example 522

### 4-(6-(2-Methylthiazol-4-ylmethoxy)-1H-benzo[d]imidazol-2-yl)-N-(4-morpholinophenyl)

benzamide (Compound 622)

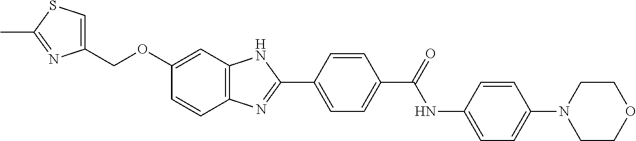 [SMILES: Cc1nc(COc2ccc3nc(-c4ccc(C(=O)Nc5ccc(N6CCOCC6)cc5)cc4)[nH]c3c2)cs1]

Compound 622 was prepared according to the procedure similar to that described in Scheme III from 1-(2-methylthiazol-4-yl)methoxy-3,4-dinitrobenzene and 4-(4-(4-morpholinophenyl)aminocarbonylbenzaldehyde. [M+H]⁺ calcd for C₂₉H₂₇N₅O₃S: 526.18. Found: 526.06.

### Example 523

### 4-(Cyclopropanecarboxamido)-N-(4-(N-(4-(4-(cyclopropanecarboxamido)benzamido)phenyl)cyclopanecarboxamido)phenyl)benzamide (Compound 623)

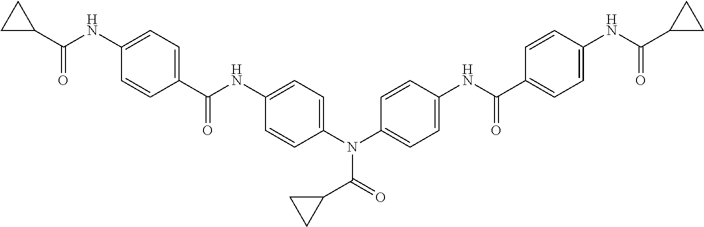 [SMILES: O=C(Nc1ccc(N(C(=O)C2CC2)c2ccc(NC(=O)c3ccc(NC(=O)C4CC4)cc3)cc2)cc1)c1ccc(NC(=O)C2CC2)cc1]

Compound 623 was prepared by a general amide formation reaction from cyclopropanecarboxylic acid and compound 580. [M+H]⁺ calcd for C₃₈H₃₅N₅O₅: 642.26. Found 642.19.

### Example 524

### 4-(6-(Pyridin-2-ylmethoxy)-1H-benzo[d]imidazol-2-yl)-N-(4-morpholinophenyl)benzamide (Compound 624)

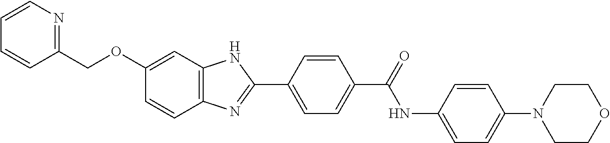 [SMILES: O=C(Nc1ccc(N2CCOCC2)cc1)c1ccc(-c2nc3ccc(OCc4ccccn4)cc3[nH]2)cc1]

Compound 624 was prepared according to the procedure similar to that described in Scheme III from 1-(pyridin-2-ylmethoxy)-3,4-dinitrobenzene and 4-(4-(4-morpholinophenyl)aminocarbonylbenzaldehyde. [M+H]⁺ calcd for C₃₀H₂₇N₅O₃: 506.21. Found: 506.06.

### Example 525

### 4-(Dimethylamino)-N-(4-(4-(6-methoxy-1H-benzo[d]imidazol-2-yl)benzamido)phenyl)benzamide (Compound 625)

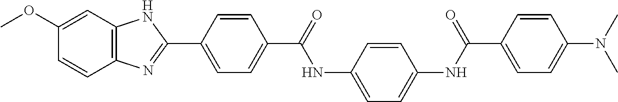

Compound 625 was prepared according to the procedure similar to that described in Scheme III from 4-(6-methoxy-1H-benzo[d]imidazol-2-yl)benzoic acid, 1,4-phenylenediamine, and 4-dimethylaminobenzoic acid. [M+H]⁺ calcd for C₃₀H₂₇N₅O₃: 506.21. Found: 506.05.

### Example 526

### 4-(Dimethylamino)-N-(4-(6-(4-(dimethylamino)benzamido)-1H-indol-3-yl)phenyl)benzamide (Compound 626)

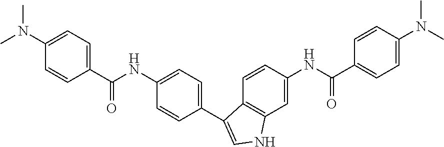

Compound 626 was prepared according to the procedure described in Scheme IV from 6-amino-3-(4-aminophenyl)-1H-indole and 4-dimethylaminobenzoate. [M+H]⁺ calcd for C₃₂H₃₁N₅O₂: 518.15. Found: 518.09.

### Example 527

### N,N′-((Methylazanediyl)bis(4,1-phenylene))bis(4-dimethylaminobenzamide) (Compound 627)

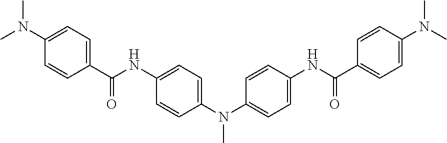

Compound 627 was prepared by reductive methylation of compound 580.

### Example 528

### 4-(6-((3-Methyl-1,2,4-oxadiazol-5-yl)methoxy)-1H-benzo[d]imidazol-2-yl)-N-(4-morpholinophenyl)benzamide (Compound 628)

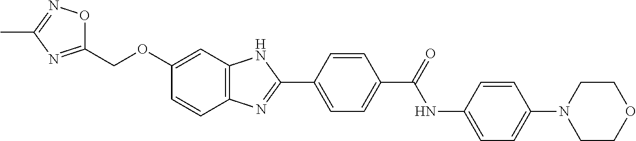 [SMILES: Cc1noc(COc2ccc3nc(-c4ccc(C(=O)Nc5ccc(N6CCOCC6)cc5)cc4)[nH]c3c2)n1]

Compound 628 was prepared according to the procedure similar to that described in Scheme III from 1-(3-methyl-1,2,4-oxadiazol-5-yl)methoxy-3,4-dinitrobenzene and 4-(4-(4-morpholinophenyl)aminocarbonylbenzaldehyde. [M+H]calcd for C₂₈H₂₆N₆O₄: 511.20. Found: 511.04.

### Example 529

### 4-(6-((5-Methylisoxazol-3-yl)methoxy)-1H-benzo[d]imidazol-2-yl)-N-(4-morpholinophenyl)benzamide (Compound 629)

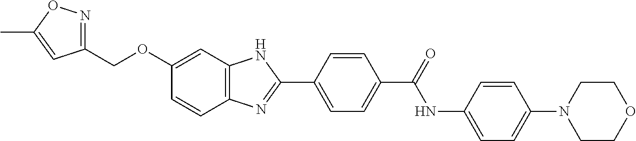 [SMILES: Cc1cc(COc2ccc3nc(-c4ccc(C(=O)Nc5ccc(N6CCOCC6)cc5)cc4)[nH]c3c2)no1]

Compound 629 was prepared according to the procedure similar to that described in Scheme III from 1-(5-methylisoxazol-3-yl)methoxy-3,4-dinitrobenzene and 4-(4-(4-morpholinophenyl)aminocarbonylbenzaldehyde. [M+H]⁺ calcd for C₂₉H₂₇N₅O₄: 510.21. Found: 510.06.

### Example 530

### 4-(6-((5-Cyclopropyl-1,3,4-oxadiazol-2-yl)methoxy)-1H-benzo[d]imidazol-2-yl)-N-(4-morpholinophenyl)benzamide (Compound 630)

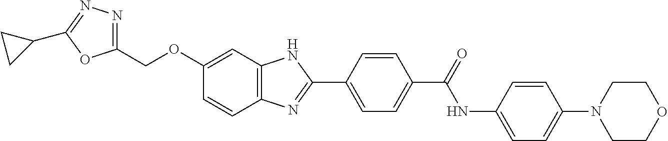 [SMILES: O=C(Nc1ccc(N2CCOCC2)cc1)c1ccc(-c2nc3ccc(OCc4nnc(C5CC5)o4)cc3[nH]2)cc1]

Compound 630 was prepared according to the procedure similar to that described in Scheme III from 1-(5-cyclopropyl-1,3,4-oxadiazol-2-yl)methoxy-3,4-dinitrobenzene and 4-(4-(4-morpholinophenyl)aminocarbonylbenzaldehyde. [M+H]⁺ calcd for C₃₀H₂₈N₆O₄: 537.22. Found: 537.06.

### Example 531

### 4-(Dimethylamino)-N-(2-(4-dimethylaminophenyl)-1H-imidazo[4,5-b]pyridin-6-yl)benzamide (Compound 631)

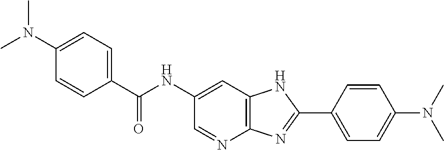

Compound 631 was prepared according to the procedure similar to that described in Scheme III from 4-dimethylaminobenzoic acid and 6-amino-2-(4-dimethylaminophenyl)-1H-imidazo[4,5-b]pyridine. [M+H]⁺ calcd for C₂₃H₂₄N₆O: 401.20. Found 401.07.

### Example 532

### 4-(6-Allyloxy-1H-benzo[d]imidazol-2-yl)-N-(4-morpholinophenyl)benzamide (Compound 632)

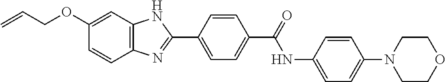 [SMILES: C=CCOc1ccc2nc(-c3ccc(C(=O)Nc4ccc(N5CCOCC5)cc4)cc3)[nH]c2c1]

Compound 632 was prepared according to the procedure similar to that described in Scheme III from 1-allyloxy-3,4-dinitrobenzene and 4-(4-(4-morpholinophenyl)aminocarbonylbenzaldehyde. [M+H]⁺ calcd for C₂₇H₂₆N₄O₃: 455.21. Found: 455.00.

### Example 533

### 2-(4-Methoxyphenyl)-5-methyl-N-(2-(4-morpholinophenyl)-1H-benzo[d]imidazol-6-yl)-1H-imidazole-4-carboxamide (Compound 633)

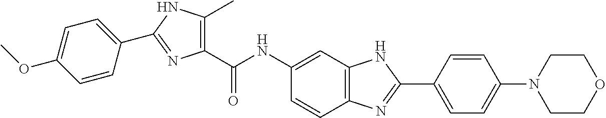 [SMILES: COc1ccc(-c2nc(C(=O)Nc3ccc4nc(-c5ccc(N6CCOCC6)cc5)[nH]c4c3)c(C)[nH]2)cc1]

Compound 633 was prepared according to the procedure similar to that described in Scheme III from 2-(4-methoxyphenyl)-1H-imidazole-4-carboxylic acid and 6-amino-2-(4-morpholinophenyl)benzimidazole. [M+H]⁺ calcd for C₂₉H₂₈N₆O₃: 509.22. Found 509.11.

### Example 534

### 4-(5-Methoxy-1H-imidazo[4,5-b]pyridin-2-yl)-N-(4-morpholinophenyl)benzamide (Compound 632)

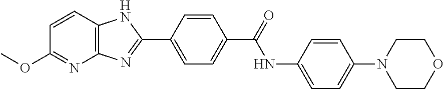 [SMILES: COc1ccc2[nH]c(-c3ccc(C(=O)Nc4ccc(N5CCOCC5)cc4)cc3)nc2n1]

Compound 634 was prepared according to the procedure similar to that described in Scheme III from 4-(5-methoxy-1H-imidazo[4,5-b]pyridin-2-yl)benzoic acid and 4-morpholinoaniline. [M+H]⁺ calcd for C₂₄H₂₃N₅O₃: 430.18. Found 429.96.

### Example 535

### N-Cyclopropyl-4-(6-methoxy-1H-benzo[d]imidazol-2-yl)benzamide (Compound 635)

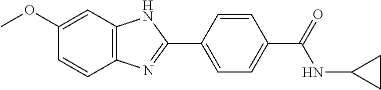 [SMILES: COc1ccc2nc(-c3ccc(C(=O)NC4CC4)cc3)[nH]c2c1]

Compound 635 was prepared according to the procedure similar to that described in Scheme III from 1-methoxy-3,4-dinitrobenzene and 4-cyclopropylaminocarbonylbenzaldehyde. [M+H]⁺ calcd for C₁₈H₁₇N₃O₂: 308.14. Found: 308.16.

### Example 536

### N-(1H-indazol-5-yl)-4-(6-methoxy-1H-benzo[d]imidazol-2-yl)benzamide (Compound 636)

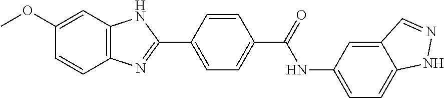 [SMILES: COc1ccc2nc(-c3ccc(C(=O)Nc4ccc5[nH]ncc5c4)cc3)[nH]c2c1]

Compound 636 was prepared according to the procedure similar to that described in Scheme III from 1-methoxy-3,4-dinitrobenzene and 4-(1H-indazol-5-yl)aminocarbonyl)benzaldehyde. [M+H]⁺ calcd for C₂₂H₁₇N₅O₂: 384.15. Found: 384.20.

### Example 537

### 4-(6-(2-Methoxyethyloxy)-1H-benzo[d]imidazol-2-yl)-N-(4-morpholinophenyl)benzamide (Compound 637)

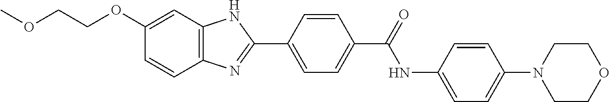 [SMILES: COCCOc1ccc2nc(-c3ccc(C(=O)Nc4ccc(N5CCOCC5)cc4)cc3)[nH]c2c1]

Compound 637 was prepared according to the procedure similar to that described in Scheme III from 1-(2-methoxyethyloxy)-3,4-dinitrobenzene and 4-(4-(4-morpholinophenyl)aminocarbonylbenzaldehyde. [M+H]⁺ calcd for C₂₇H₂₈N₄O₄: 473.21. Found: 473.08.

### Example 538

### 4-(5-(Benzo[d]thiazol-2-yl)-1H-benzo[d]imidazol-2-yl)-N-(4-morpholinophenyl)benzamide (Compound 638)

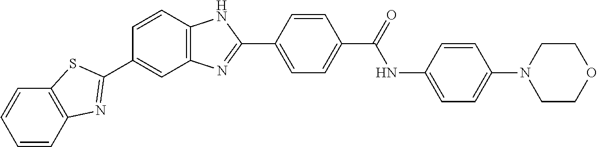 [SMILES: O=C(Nc1ccc(N2CCOCC2)cc1)c1ccc(-c2nc3cc(-c4nc5ccccc5s4)ccc3[nH]2)cc1]

Compound 638 was prepared according to the procedure similar to that described in Scheme III from 1-(benzothiazol-2-yl)-3,4-dinitrobenzene and 4-(4-(4-morpholinophenyl)aminocarbonylbenzaldehyde. [M+H]calcd for C₃₁H₂₅N₅O₂S: 532.18. Found: 532.07.

### Example 539

### 4-(6-Benzyloxy-1H-benzo[d]imidazol-2-yl)-N-(4-morpholinophenyl)benzamide (Compound 639)

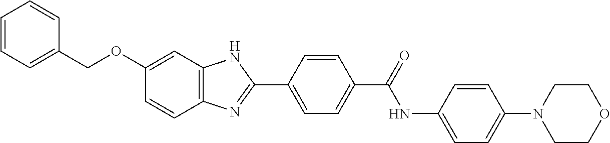 [SMILES: O=C(Nc1ccc(N2CCOCC2)cc1)c1ccc(-c2nc3ccc(OCc4ccccc4)cc3[nH]2)cc1]

Compound 639 was prepared according to the procedure similar to that described in Scheme III from 1-benzyloxy-3,4-dinitrobenzene and 4-(4-(4-morpholinophenyl)aminocarbonylbenzaldehyde. [M+H]⁺ calcd for C₂₇H₂₈N₄O₄: 473.21. Found: 473.08.

### Example 540

### 4-(6-Cyclopropylmethoxy-1H-benzo[d]imidazol-2-yl)-N-(4-morpholinophenyl)benzamide (Compound 640)

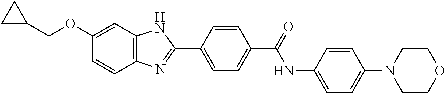 [SMILES: O=C(Nc1ccc(N2CCOCC2)cc1)c1ccc(-c2nc3ccc(OCC4CC4)cc3[nH]2)cc1]

Compound 640 was prepared according to the procedure similar to that described in Scheme III from 1-cyclorpopylmethoxy-3,4-dinitrobenzene and 4-(4-(4-morpholinophenyl)aminocarbonylbenzaldehyde. [M+H]⁺ calcd for C₂₈H₂₈N₄O₃: 469.22. Found: 469.03.

### Example 541

### 4-(6-(4-Cyanobenzyloxy)-1H-benzo[d]imidazol-2-yl)-N-(4-morpholinophenyl)benzamide (Compound 641)

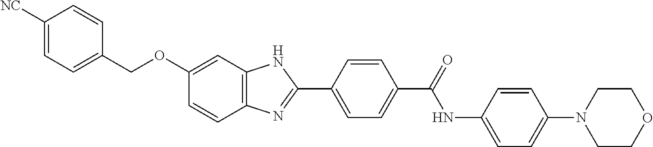 [SMILES: N#Cc1ccc(COc2ccc3nc(-c4ccc(C(=O)Nc5ccc(N6CCOCC6)cc5)cc4)[nH]c3c2)cc1]

Compound 641 was prepared according to the procedure similar to that described in Scheme III from 1-(4-cyanobenzyloxy)-3,4-dinitrobenzene and 4-(4-(4-morpholinophenyl)aminocarbonylbenzaldehyde. [M+H]⁺ calcd for C₃₂H₂₇N₅O₃: 530.21. Found: 530.10.

### Example 542

### 4-(6-(2-(Morpholinoethyloxy)-1H-benzo[d]imidazol-2-yl)-N-(4-morpholinophenyl)benzamide (Compound 642)

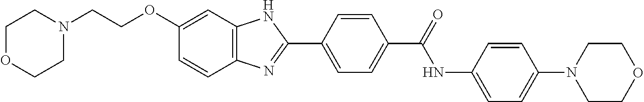 [SMILES: O=C(Nc1ccc(N2CCOCC2)cc1)c1ccc(-c2nc3ccc(OCCN4CCOCC4)cc3[nH]2)cc1]

Compound 642 was prepared according to the procedure similar to that described in Scheme III from 1-(2-morpholinoethyloxy)-3,4-dinitrobenzene and 4-(4-(4-morpholinophenyl)aminocarbonylbenzaldehyde. [M+H]⁺ calcd for C₃₀H₃₃N₅O₄: 528.25. Found: 528.15.

### Example 543

### 4-(6-(2-(Diethylamino)ethyloxy)-1H-benzo[d]imidazol-2-yl)-N-(4-morpholinophenyl)benzamide (Compound 643)

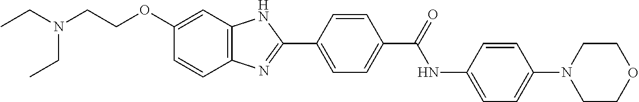 [SMILES: CCN(CC)CCOc1ccc2nc(-c3ccc(C(=O)Nc4ccc(N5CCOCC5)cc4)cc3)[nH]c2c1]

Compound 643 was prepared according to the procedure similar to that described in Scheme III from 1-(2-diethylaminoethyloxy)-3,4-dinitrobenzene and 4-(4-(4-morpholinophenyl)aminocarbonylbenzaldehyde. [M+H]⁺ calcd for C₃₀H₃₅N₅O₃: 514.27. Found: 514.13.

### Example 544

### (N-(4-(6-Phenyl-1H-benzo[d]imidazol-2-yl)phenyl)-1H-pyrrole-2-carboxamide (Compound 644)

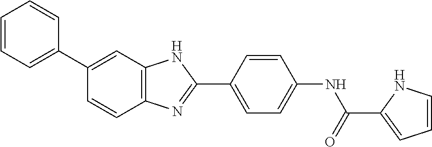 [SMILES: O=C(Nc1ccc(-c2nc3ccc(-c4ccccc4)cc3[nH]2)cc1)c1ccc[nH]1]

Compound 644 was prepared according to the procedure similar to that described in Scheme III from 1-phenyl-3,4-dinitrobenzene, 4-nitrobenzaldehyde, and 2-pyrrolecarboxylic acid. [M+H]⁺ calcd for C₂₄H₁₈N₄O: 379.15. Found: 378.96.

### Example 545

### 1-Methyl-(N-(4-(6-phenyl-1H-benzo[d]imidazol-2-yl)phenyl)-1H-pyrrole-2-carboxamide (Compound 645)

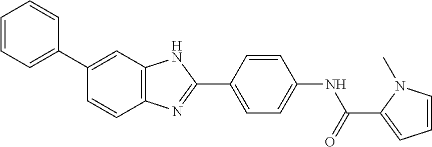 [SMILES: Cn1cccc1C(=O)Nc1ccc(-c2nc3ccc(-c4ccccc4)cc3[nH]2)cc1]

Compound 645 was prepared according to the procedure similar to that described in Scheme III from 1-phenyl-3,4-dinitrobenzene, 4-nitrobenzaldehyde, and 1-methylpyrrole-2-carboxylic acid. [M+H]⁺ calcd for C₂₅H₂₀N₄O: 393.17. Found: 392.99.

### Example 546

### (4-(6-(3-Hydroxyphenyl)-1H-benzo[d]imidazol-2-yl)-N-1H-indol-6-yl)benzamide (Compound 646)

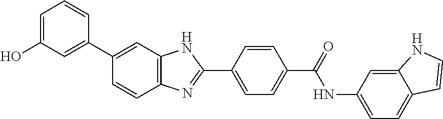 [SMILES: O=C(Nc1ccc2cc[nH]c2c1)c1ccc(-c2nc3ccc(-c4cccc(O)c4)cc3[nH]2)cc1]

Compound 646 was prepared according to the procedure similar to that described in Scheme III from 1-(3-hydroxyphenyl)-3,4-dinitrobenzene and 4-(6-indolylaminocarbonyl)benzaldehyde. [M+H]⁺ calcd for C₂₈H₂₀N₄O₂: 445.16. Found: 445.00.

### Example 547

### (4-(6-(4-Hydroxyphenyl)-1H-benzo[d]imidazol-2-yl)-N-1H-indol-6-yl)benzamide (Compound 647)

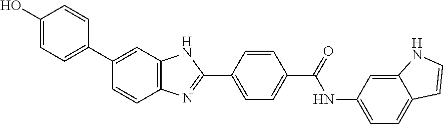 [SMILES: O=C(Nc1ccc2cc[nH]c2c1)c1ccc(-c2nc3ccc(-c4ccc(O)cc4)cc3[nH]2)cc1]

Compound 647 was prepared according to the procedure similar to that described in Scheme III from 1-(4-hydroxyphenyl)-3,4-dinitrobenzene and 4-(6-indolylaminocarbonyl)benzaldehyde. [M+H]⁺ calcd for C₂₈H₂₀N₄O₂: 445.16. Found: 445.00.

### Example 548

### (4-(6-(4-Methoxyphenyl)-1H-benzo[d]imidazol-2-yl)-N-1H-indol-6-yl)benzamide (Compound 648)

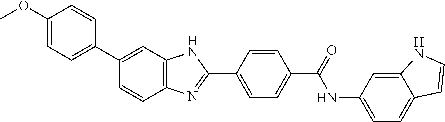 [SMILES: COc1ccc(-c2ccc3nc(-c4ccc(C(=O)Nc5ccc6cc[nH]c6c5)cc4)[nH]c3c2)cc1]

Compound 648 was prepared according to the procedure similar to that described in Scheme III from 1-(4-methoxyphenyl)-3,4-dinitrobenzene and 4-(6-indolylaminocarbonyl)benzaldehyde. [M+H]⁺ calcd for C₂₉H₂₂N₄O₂: 459.17. Found: 458.97.

### Example 549

### (4-(6-(4-Hydroxyphenyl)-1H-benzo[d]imidazol-2-yl)-N-(4-morpholinophenyl)benzamide (Compound 649)

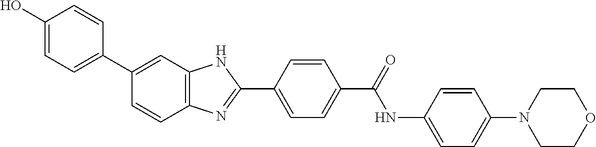 [SMILES: O=C(Nc1ccc(N2CCOCC2)cc1)c1ccc(-c2nc3ccc(-c4ccc(O)cc4)cc3[nH]2)cc1]

Compound 649 was prepared according to the procedure similar to that described in Scheme III from 1-(4-hydroxyphenyl)-3,4-dinitrobenzene and 4-(4-morpholinopheyl)aminocarbonyl)benzaldehyde. [M+H]⁺ calcd for C₃₀H₂₆N₄O₃: 491.20. Found: 491.03.

### Example 550

### N-(1H-indol-6-yl)-4-(6-(pyridin-3-yl)-1H-benzo[d]imidazol-2-yl)-benzamide (Compound 650)

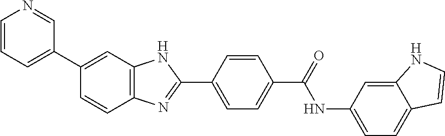 [SMILES: O=C(Nc1ccc2cc[nH]c2c1)c1ccc(-c2nc3ccc(-c4cccnc4)cc3[nH]2)cc1]

Compound 650 was prepared according to the procedure similar to that described in Scheme III from 1-(3-pyridinyl)-3,4-dinitrobenzene and 4-(6-indolylaminocarbonyl)benzaldehyde. [M+H]⁺ calcd for C₂₇H₁₉N₅O: 430.16. Found: 429.96.

### Example 551

### 4-(6-(3,5-Dimethylisoxazol-4-yl)-1H-benzo[d]imidazol-2-yl)-N-(1H-indol-6-yl)benzamide (Compound 651)

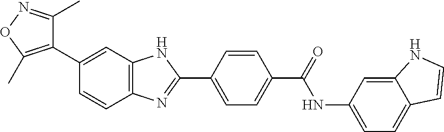 [SMILES: Cc1noc(C)c1-c1ccc2nc(-c3ccc(C(=O)Nc4ccc5cc[nH]c5c4)cc3)[nH]c2c1]

Compound 651 was prepared according to the procedure similar to that described in Scheme III from 1-(3,5-dimethylisoxazol-4-yl)-3,4-dinitrobenzene and 4-(6-indolylaminocarbonyl)benzaldehyde. [M+H]⁺ calcd for C₂₇H₂₁N₅O₂: 448.17. Found: 447.97.

### Example 552

### (4-(6-(4-Methoxyphenyl)-1H-benzo[d]imidazol-2-yl)-N-(4-morpholinophenyl)benzamide (Compound 652)

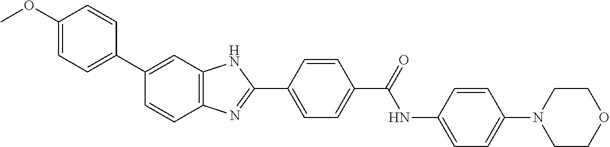 [SMILES: COc1ccc(-c2ccc3nc(-c4ccc(C(=O)Nc5ccc(N6CCOCC6)cc5)cc4)[nH]c3c2)cc1]

Compound 652 was prepared according to the procedure similar to that described in Scheme III from 1-(4-methoxyphenyl)-3,4-dinitrobenzene and 4-(4-morpholinopheyl)aminocarbonyl)benzaldehyde. [M+H]⁺ calcd for C₃₁H₂₈N₄O₃: 505.22. Found: 505.07.

### Example 553

### 4-(6-((5-Cyanothiophen-2-yl)methoxy)-1H-benzo[d]imidazol-2-yl)-N-(4-morpholinophenyl)benzamide (Compound 653)

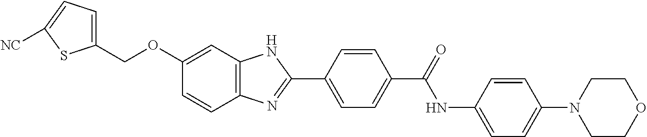 [SMILES: N#Cc1ccc(COc2ccc3nc(-c4ccc(C(=O)Nc5ccc(N6CCOCC6)cc5)cc4)[nH]c3c2)s1]

Compound 653 was prepared according to the procedure similar to that described in Scheme III from 1-(5-cyanothiophen-2-yl)methoxy)-3,4-dinitrobenzene and 4-(4-morpholinopheyl)aminocarbonyl)benzaldehyde. [M+H]⁺ calcd for C₃₀H₂₅N₅O₃S: 536.17. Found: 536.05.

### Example 554

### N-(1H-Indol-5-yl)-4-(6-methoxy-1H-benzo[d]imidazol-2-yl)benzamide (Compound 654)

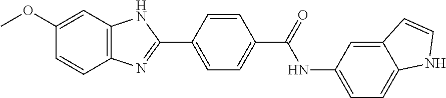 [SMILES: COc1ccc2nc(-c3ccc(C(=O)Nc4ccc5[nH]ccc5c4)cc3)[nH]c2c1]

Compound 654 was prepared according to the procedure similar to that described in Scheme III from 3,4-dinitroanisole and 4-(5-indolylaminocarbonyl)benzaldehyde. [M+H]⁺ calcd for C₂₃H₁₈N₄O₂: 383.14. Found: 383.01.

### Example 555

### N-(1-Methylindol-5-yl)-4-(6-methoxy-1H-benzo[d]imidazol-2-yl)benzamide (Compound 655)

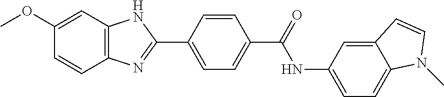

Compound 655 was prepared according to the procedure similar to that described in Scheme III from 3,4-dinitroanisole and 4-(5-(1-methylindolyl)aminocarbonyl)benzaldehyde. [M+H]⁺ calcd for C₂₄H₂₀N₄O₂: 397.16. Found: 396.97.

### Example 556

### 4-(6-((3-Methyl-1,2,4-oxadiazol-5-yl)methoxy)-1H-benzo[d]imidazol-2-yl)-N-(1-methyl-1H-indol-5-yl)benzamide (Compound 656)

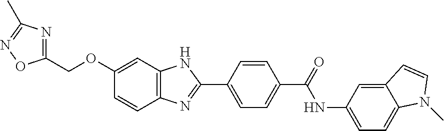 [SMILES: Cc1noc(COc2ccc3nc(-c4ccc(C(=O)Nc5ccc6c(ccn6C)c5)cc4)[nH]c3c2)n1]

Compound 656 was prepared according to the procedure similar to that described in Scheme III from 1-(3-methyl-1,2,4-oxadiazol-5-yl)methoxy-3,4-dinitrobenzene and 4-(5-(1-methylindolyl)aminocarbonyl)benzaldehyde. [M+H]⁺ calcd for C₂₇H₂₂N₆O₃: 479.18. Found: 479.01.

### Example 557

### N-(4-(1,4-Dioxa-8-azaspiro[4.5]decan-8-yl)phenyl)-4-(6-methoxy-1H-benzo[d]imidazol-2-yl)benzamide (Compound 657)

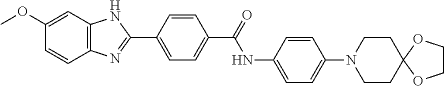 [SMILES: COc1ccc2nc(-c3ccc(C(=O)Nc4ccc(N5CCC6(CC5)OCCO6)cc4)cc3)[nH]c2c1]

Compound 657 was prepared according to the procedure similar to that described in Scheme III from 3,4-dinitroanisole and 4-(4-(1,4-dioxa-8-azaspiro[4.5]decan-8-yl)phenyl)aminocarbonyl)benzaldehyde. [M+H]⁺ calcd for C₂₈H₂₈N₄O₄: 485.21. Found: 485.09.

### Example 558

### 4-(6-Methoxy-1H-benzo[d]imidazol-2-yl)-N-(1-methylindolin-5-yl)benzamide (Compound 658)

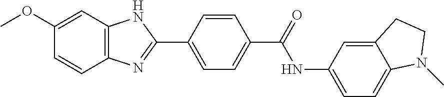

Compound 658 was prepared according to the procedure similar to that described in Scheme III from 3,4-dinitroanisole and 4-(5-(1-methylindolinyl)aminocarbonyl)benzaldehyde. [M+H]⁺ calcd for C₂₄H₂₂N₄O₂: 399.17. Found: 398.99.

### Example 559

### 4-(6-((3-Methyl-1,2,4-oxadiazol-5-yl)methoxy)-1H-benzo[d]imidazol-2-yl)-N-(1H-indol-5-yl)benzamide (Compound 659)

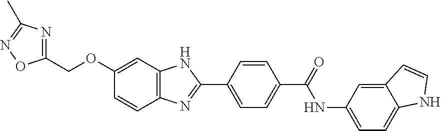 [SMILES: Cc1noc(COc2ccc3nc(-c4ccc(C(=O)Nc5ccc6[nH]ccc6c5)cc4)[nH]c3c2)n1]

Compound 659 was prepared according to the procedure similar to that described in Scheme III from 1-(3-methyl-1,2,4-oxadiazol-5-yl)methoxy-3,4-dinitrobenzene and 4-(5-indolylaminocarbonyl)benzaldehyde. [M+H]⁺ calcd for C₂₆H₂₀N₆O₃: 465.16. Found: 464.98.

### Example 560

### 4-(6-((3-Methyl-1,2,4-oxadiazol-5-yl)methoxy)-1H-benzo[d]imidazol-2-yl)-N-(1H-indol-6-yl)benzamide (Compound 660)

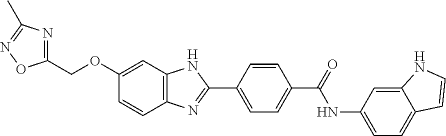 [SMILES: Cc1noc(COc2ccc3nc(-c4ccc(C(=O)Nc5ccc6cc[nH]c6c5)cc4)[nH]c3c2)n1]

Compound 660 was prepared according to the procedure similar to that described in Scheme III from 1-(3-methyl-1,2,4-oxadiazol-5-yl)methoxy-3,4-dinitrobenzene and 4-(6-indolylaminocarbonyl)benzaldehyde. [M+H]⁺ calcd for C₂₆H₂₀N₆O₃: 465.16. Found: 465.04.

### Example 561

### 4-(6-(2-Methoxyethoxy)-1H-benzo[d]imidazol-2-yl)-N-(1H-indol-5-yl)benzamide (Compound 661)

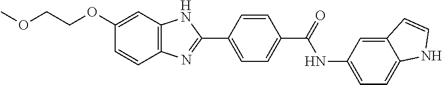 [SMILES: COCCOc1ccc2nc(-c3ccc(C(=O)Nc4ccc5[nH]ccc5c4)cc3)[nH]c2c1]

Compound 661 was prepared according to the procedure similar to that described in Scheme III from 1-(2-methoxyethoxy-3,4-dinitrobenzene and 4-(5-indolylaminocarbonyl)benzaldehyde. [M+H]⁺ calcd for C₂₅H₂₂N₄O₃: 427.17. Found: 426.99.

### Example 562

### 4-(6-(2-Methoxyethoxy)-1H-benzo[d]imidazol-2-yl)-N-(2-methyl-1H-indol-5-yl)benzamide (Compound 662)

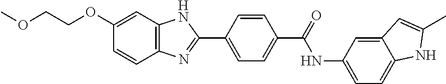

Compound 662 was prepared according to the procedure similar to that described in Scheme III from 1-(2-methoxyethoxy-3,4-dinitrobenzene and 4-(2-methyl-5-indolylaminocarbonyl)benzaldehyde. [M+H]⁺ calcd for C₂₆H₂₄N₄O₃: 441.18. Found: 441.02.

### Example 563

### 4-(6-(2-Methoxyethoxy)-1H-benzo[d]imidazol-2-yl)-N-(1H-indol-2-yl)benzamide (Compound 663)

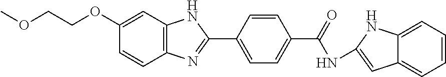 [SMILES: COCCOc1ccc2nc(-c3ccc(C(=O)Nc4cc5ccccc5[nH]4)cc3)[nH]c2c1]

Compound 663 was prepared according to the procedure similar to that described in Scheme III from 1-(2-methoxyethoxy-3,4-dinitrobenzene and 4-(2-indolylaminocarbonyl)benzaldehyde. [M+H]⁺ calcd for C₂₅H₂₂N₄O₃: 427.17. Found: 426.99.

### Example 564

### 4-(6-(2-Methoxyethoxy)-1H-benzo[d]imidazol-2-yl)-N-(1H-indol-6-yl)benzamide (Compound 664)

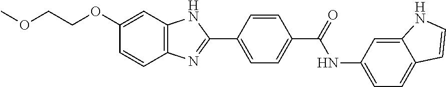 [SMILES: COCCOc1ccc2nc(-c3ccc(C(=O)Nc4ccc5cc[nH]c5c4)cc3)[nH]c2c1]

Compound 664 was prepared according to the procedure similar to that described in Scheme III from 1-(2-methoxyethoxy-3,4-dinitrobenzene and 4-(6-indolylaminocarbonyl)benzaldehyde. [M+H]⁺ calcd for C₂₅H₂₂N₄O₃: 427.17. Found: 426.99.

### Example 565

### 4-(6-(2-Methoxyethoxy)-1H-benzo[d]imidazol-2-yl)-N-(1H-pyrrolo[2,3-b]pyridin-5-yl)benzamide (Compound 665)

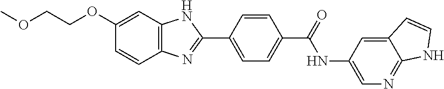 [SMILES: COCCOc1ccc2nc(-c3ccc(C(=O)Nc4cnc5[nH]ccc5c4)cc3)[nH]c2c1]

Compound 665 was prepared according to the procedure similar to that described in Scheme III from 1-(2-methoxyethoxy-3,4-dinitrobenzene and 4-(1H-pyrrolo[2,3-b]pyridin-5-yl)aminocarbonyl)benzaldehyde. ¹H NMR (500 MHz, CD₃OD) δ 8.45 (s, 1H), 8.35 (s, 1H), 8.19 (d, J=8.5 Hz, 2H), 8.15 (d, J=8.5 Hz, 2H), 7.55 (d, J=9 Hz, 1H), 7.42 (d, J=3.5 Hz, 1H), 7.15 (s, 1H), 7.05 (d, J=9 Hz, 1H), 6.51 (d, J=3.5 Hz, 1H), 4.19 (t, J=4.5 Hz, 2H), 3.79 (t, J=4.5 Hz, 2H), 3.31 (s, 3H).

### Example 566

### 4-(6-(2-Methoxyethoxy)-1H-benzo[d]imidazol-2-yl)-N-(2-oxoindolin-5-yl)benzamide (Compound 666)

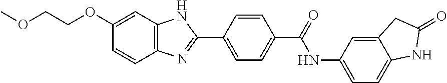 [SMILES: COCCOc1ccc2nc(-c3ccc(C(=O)Nc4ccc5c(c4)CC(=O)N5)cc3)[nH]c2c1]

Compound 666 was prepared according to the procedure similar to that described in Scheme III from 1-(2-methoxyethoxy-3,4-dinitrobenzene and 4-((2-oxoindolin-5-yl)aminocarbonyl)benzaldehyde. [M+H]⁺ calcd for C₂₅H₂₂N₄O₄: 443.16. Found: 443.05.

### Example 567

### 4-(6-(2-Methoxyethoxy)-1H-benzo[d]imidazol-2-yl)-N-(1H-pyrrolo[2,3-b]pyridin-6-yl)benzamide (Compound 667)

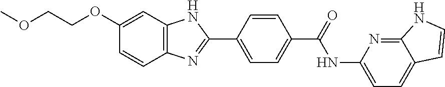 [SMILES: COCCOc1ccc2nc(-c3ccc(C(=O)Nc4ccc5cc[nH]c5n4)cc3)[nH]c2c1]

Compound 667 was prepared according to the procedure similar to that described in Scheme III from 1-(2-methoxyethoxy-3,4-dinitrobenzene and 4-(1H-pyrrolo[2,3-b]pyridin-6-yl)aminocarbonyl)benzaldehyde. [M+H]⁺ calcd for C₂₄H₂₁N₅O₃: 428.17. Found: 428.00.

### Example 568

### 4-(6-(2-Methoxyethoxy)-1H-benzo[d]imidazol-2-yl)-N-(1H-pyrrolo[3,2-c]pyridin-6-yl)benzamide (Compound 668)

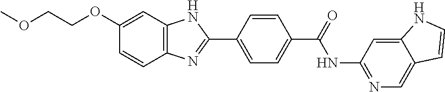 [SMILES: COCCOc1ccc2nc(-c3ccc(C(=O)Nc4cc5[nH]ccc5cn4)cc3)[nH]c2c1]

Compound 668 was prepared according to the procedure similar to that described in Scheme III from 1-(2-methoxyethoxy-3,4-dinitrobenzene and 4-(1H-pyrrolo[3,2-c]pyridin-6-yl)aminocarbonyl)benzaldehyde. [M+H]⁺ calcd for C₂₄H₂₁N₅O₃: 428.17. Found: 428.07.

### Example 569

### 4-(6-((4-Cyanobenzyl)oxy)-1H-benzo[d]imidazol-2-yl)-N-cyclopropylbenzamide (Compound 669)

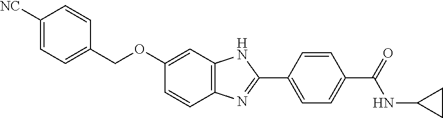 [SMILES: N#Cc1ccc(COc2ccc3nc(-c4ccc(C(=O)NC5CC5)cc4)[nH]c3c2)cc1]

Compound 669 was prepared according to the procedure similar to that described in Scheme III from 1-(4-cyanobenzyl)oxy-3,4-dinitrobenzene and 4-(cyclopropylaminocarbonyl)benzaldehyde. [M+H]⁺ calcd for C₂₅H₂₀N₄O₂: 409.16. Found: 409.04.

### Example 570

### N-(4-(4-Hydroxypiperidin-1-yl)phenyl)-4-(6-(2-methoxyethoxy)-1H-benzo[d]imidazol-2-yl)benzamide (Compound 670)

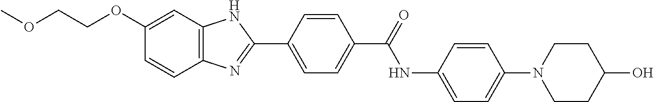 [SMILES: COCCOc1ccc2nc(-c3ccc(C(=O)Nc4ccc(N5CCC(O)CC5)cc4)cc3)[nH]c2c1]

Compound 670 was prepared according to the procedure similar to that described in Scheme III from 1-(2-methoxyethoxy-3,4-dinitrobenzene and 4-(4-(4-hydroxylpiperidin-1-yl)phenyl)aminocarbonyl)benzaldehyde. [M+H]calcd for C₂₈H₃₀N₄O₄: 487.23. Found: 487.05.

### Example 571

### 4-(6-((5-Cyanothiophen-2-yl)methoxy)-1H-benzo[d]imidazol-2-yl)-N-(1H-indol-6-yl)benzamide (Compound 671)

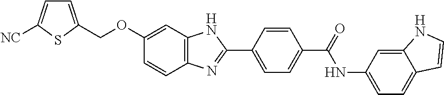 [SMILES: N#Cc1ccc(COc2ccc3nc(-c4ccc(C(=O)Nc5ccc6cc[nH]c6c5)cc4)[nH]c3c2)s1]

Compound 671 was prepared according to the procedure similar to that described in Scheme III from 1-(5-cyanothiophen-2-yl)methoxy)-3,4-dinitrobenzene and 4-(indol-6-yl)aminocarbonyl)benzaldehyde. [M+H]⁺ calcd for C₂₈H₁₉N₅O₂S: 490.13. Found: 489.95.

### Example 572

### 4-(6-(2-Hydroxyethoxy)-1H-benzo[d]imidazol-2-yl)-N-(1H-indol-6-yl)benzamide (Compound 672)

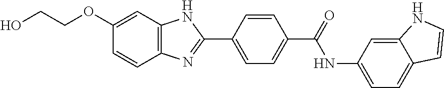 [SMILES: O=C(Nc1ccc2cc[nH]c2c1)c1ccc(-c2nc3ccc(OCCO)cc3[nH]2)cc1]

Compound 672 was prepared according to the procedure similar to that described in Scheme III from 1-(2-hydroxyethoxy)-3,4-dinitrobenzene and 4-(6-indolylaminocarbonyl)benzaldehyde. [M+H]⁺ calcd for C₂₄H₂₀N₄O₃: 413.15. Found: 412.96.

### Example 573

### Methyl 2-((2-(4-((1H-indol-6-yl)carbamoyl)phenyl)-1H-benzo[d]imidazol-6-yl)oxy)acetate (Compound 673)

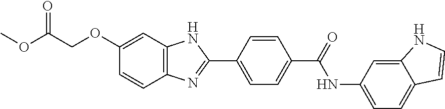 [SMILES: COC(=O)COc1ccc2nc(-c3ccc(C(=O)Nc4ccc5cc[nH]c5c4)cc3)[nH]c2c1]

Compound 673 was prepared according to the procedure similar to that described in Scheme III from 1-(methoxycarbanylmethoxy)-3,4-dinitrobenzene and 4-(6-indolylaminocarbonyl)benzaldehyde. [M+H]⁺ calcd for C₂₅H₂₀N₄O₄: 441.15. Found: 440.96.

### Example 574

### 4-(6-(2-Methoxyethoxy)-1H-benzo[d]imidazol-2-yl)-N-(1-(2-hydroxyethyl)indol-5-yl)benzamide (Compound 674)

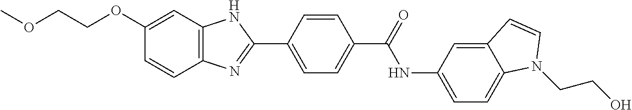 [SMILES: COCCOc1ccc2nc(-c3ccc(C(=O)Nc4ccc5c(ccn5CCO)c4)cc3)[nH]c2c1]

Compound 674 was prepared according to the procedure similar to that described in Scheme III from 1-(2-methoxyethoxy-3,4-dinitrobenzene and 4-(1-(2-hydroxyethyl)-5-indolylaminocarbonyl)benzaldehyde. [M+H]⁺ calcd for C₂₇H₂₆N₄O₄: 471.20. Found: 471.05.

### Example 575

### 4-(6-(2-Methoxyethoxy)-1H-benzo[d]imidazol-2-yl)-N-(1-(2-methoxyethyl)-2-methylindol-5-yl)benzamide (Compound 675)

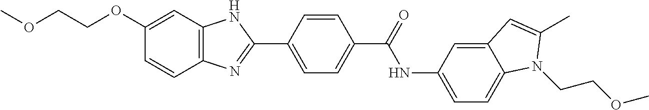

Compound 675 was prepared according to the procedure similar to that described in Scheme III from 1-(2-methoxyethoxy-3,4-dinitrobenzene and 4-(1-(2-methoxyethyl)2-methyl-5-indolylaminocarbonyl)benzaldehyde. [M+H]⁺ calcd for C₂₉H₃₀N₄O₄: 499.24. Found: 499.06.

### Example 576

### 4-(6-(2-Methoxyethoxy)-1H-benzo[d]imidazol-2-yl)-N-(1-(2-morpholinoethyl)indol-5-yl)benzamide (Compound 676)

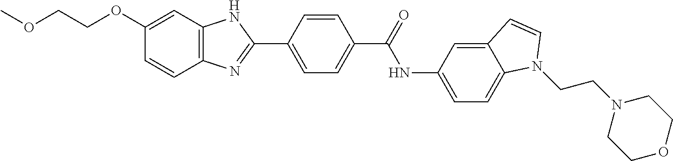 [SMILES: COCCOc1ccc2nc(-c3ccc(C(=O)Nc4ccc5c(ccn5CCN5CCOCC5)c4)cc3)[nH]c2c1]

Compound 676 was prepared according to the procedure similar to that described in Scheme III from 1-(2-methoxyethoxy-3,4-dinitrobenzene and 4-(1-(2-morpholinoethyl)-5-indolylaminocarbonyl)benzaldehyde. [M+H]⁺ calcd for C₃₁H₃₃N₅O₄: 540.26. Found: 540.10.

### Example 577

### (6-Phenyl-1H-benzo[d]imidazol-2-yl)-N-(4-morpholinophenyl)benzamide (Compound 677)

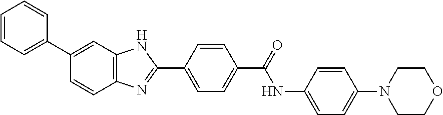 [SMILES: O=C(Nc1ccc(N2CCOCC2)cc1)c1ccc(-c2nc3ccc(-c4ccccc4)cc3[nH]2)cc1]

Compound 677 was prepared according to the procedure similar to that described in Scheme III from 1-phenyl-3,4-dinitrobenzene and 4-(4-morpholinopheyl)aminocarbonyl)benzaldehyde. [M+H]⁺ calcd for C₃₀H₂₆N₄O₂: 475.22. Found: 475.03.

### Example 578

### (6-Phenyl-1H-benzo[d]imidazol-2-yl)-N-1H-indol-6-yl)benzamide (Compound 678)

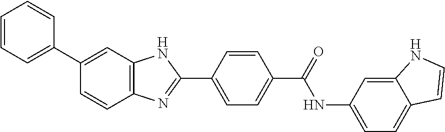 [SMILES: O=C(Nc1ccc2cc[nH]c2c1)c1ccc(-c2nc3ccc(-c4ccccc4)cc3[nH]2)cc1]

Compound 678 was prepared according to the procedure similar to that described in Scheme III from 1-phenyl-3,4-dinitrobenzene and 4-(6-indolylaminocarbonyl)benzaldehyde. [M+H]⁺ calcd for C₂₈H₂₀N₄O: 429.16. Found: 428.95.

### Example 579

### (4-(6-(3-Hydroxyphenyl)-1H-benzo[d]imidazol-2-yl)-N-(4-morpholinophenyl)benzamide (Compound 679)

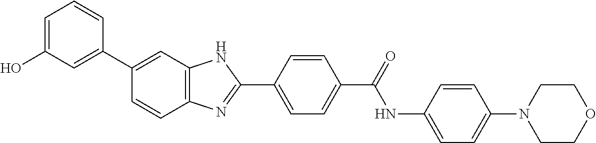 [SMILES: O=C(Nc1ccc(N2CCOCC2)cc1)c1ccc(-c2nc3ccc(-c4cccc(O)c4)cc3[nH]2)cc1]

Compound 679 was prepared according to the procedure similar to that described in Scheme III from 1-(3-hydroxyphenyl)-3,4-dinitrobenzene and 4-(4-morpholinopheyl)aminocarbonyl)benzaldehyde. [M+H]⁺ calcd for C₃₀H₂₆N₄O₃: 491.20. Found: 491.03.

### Example 580

### Cyclopropyl(4-(4-(6-(4-hydroxyphenyl)-1H-benzo[d]imidazol-2-yl)phenyl)piperazin-1-yl)methanone (Compound 680)

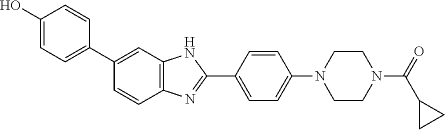 [SMILES: O=C(C1CC1)N1CCN(c2ccc(-c3nc4ccc(-c5ccc(O)cc5)cc4[nH]3)cc2)CC1]

Compound 680 was prepared according to the procedure similar to that described in Scheme III from 1-(4-hydroxyphenyl)-3,4-dinitrobenzene and 4-(4-(cyclopropylcarbonylpiperazin-1-yl)benzaldehyde. [M+H]⁺ calcd for C₂₇H₂₆N₄O₂: 439.21. Found: 438.93.

### Example 581

### N-(2-(4-(Cyclopropanecarboxamido)phenyl)-2H-indazol-5-yl)-4-(4-hydroxypiperidin-1-yl)benzamide (Compound 681)

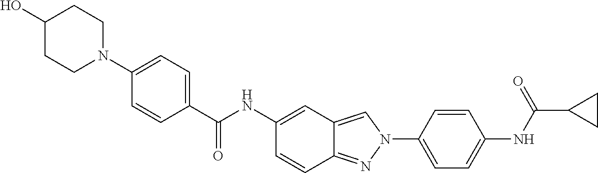 [SMILES: O=C(Nc1ccc2nn(-c3ccc(NC(=O)C4CC4)cc3)cc2c1)c1ccc(N2CCC(O)CC2)cc1]

Compound 681 was prepared according to the procedure similar to that described in Scheme IV from 4-(4-hydroxypiperidin-1-yl)benzoic acid, cyclopropanecarboxylic acid, and 2-(4-aminophenyl)-5-amino-2H-indazole. [M+H]⁺ calcd for C₂₉H₂₉N₅O₃: 496.23. Found 496.05.

### Example 582

### 4-(6-(2-Hydroxyethoxy)-1H-benzo[d]imidazol-2-yl)-N-(4-morpholinophenyl)benzamide (Compound 682)

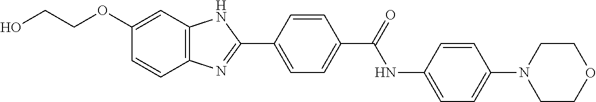 [SMILES: O=C(Nc1ccc(N2CCOCC2)cc1)c1ccc(-c2nc3ccc(OCCO)cc3[nH]2)cc1]

Compound 682 was prepared according to the procedure similar to that described in Scheme III from 1-(4-methoxyphenyl)-3,4-dinitrobenzene and 4-(4-morpholinopheyl)aminocarbonyl)benzaldehyde. [M+H]⁺ calcd for C₂₆H₂₆N₄O₄: 459.20. Found: 458.97.

### Example 583

### N-(4-(4-(Cyclopropanecarboxamido)benzoyl)phenyl)-4-(4-hydroxypiperidin-1-yl)benzamide (Compound 683)

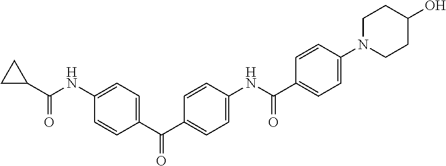 [SMILES: O=C(Nc1ccc(C(=O)c2ccc(NC(=O)C3CC3)cc2)cc1)c1ccc(N2CCC(O)CC2)cc1]

Compound 683 was prepared according to the procedure described in Scheme IV from 4,4′-diaminobenzophenone, cyclopropanecarboxylic and 4-(4-hydroxypiperidin-1-yl)benzoic acids. A solution of 4,4′-diaminobenzophenone (Aldrich, 150 mg, 0.71 mmol), 4-fluorobenzoic acid (Aldrich, 129 mg, 0.92 mmol), HATU (Aldrich, 349 mg, 0.92 mmol) and diisopropylethylamine (Aldrich, 160 μL, 0.92 mmol) in dimethylformamide (6 mL) was heated to 100° C. for 12 h. The reaction was cooled to room temperature and water (10 mL) was slowly added. The resulting solid was filtered and recrystallized from hot methanol to give N-(4-(4-aminobenzoyl)phenyl)-4-fluorobenzamide (147 mg, 0.44 mmol, 62%).

A solution of cyclopropanecarbonyl chloride (Aldrich, 448 mg, 4.31 mmol) in 5 mL of methylene chloride was slowly added over 5 minutes to N-(4-(4-aminobenzoyl)phenyl)-4-fluorobenzamide (1.2 g, 3.59 mmol) in 20 mL of methylene chloride containing 3 mL of pyridine. The reaction was stirred at room temperature for 12 h then quenched with 40 mL of water. The layers were separated and the aqueous layer was extracted with ethyl acetate (2×50 mL). The combined organics were washed with 0.5 N HCl (100 mL), brine (100 mL), dried over sodium sulphate, and filtered. Concentration of the organics provided N-(4-(4-(cyclopropanecarboxamido)benzoyl)phenyl)-4-fluorobenzamide (1.3 g, 3.23 mmol) as a tan solid which was used without further purification.

To a solution of N-(4-(4-(cyclopropanecarboxamido)benzoyl)phenyl)-4-fluorobenzamide (1.3 g, 3.23 mmol) in dimethylsulfoxide (35 mL) was added 4-hydroxypiperidine (Aldrich, 5.2 g, 51.5 mmol) in a single portion. The reaction was heated to 110° C. for 24 h then cooled to room temperature. Water (100 mL) was slowly added to the reaction at room temperature over 3 h with stirring. Upon completion the reaction was allowed to stir an additional 12 h, then filtered under vacuum. The tan precipitate was washed with water (50 mL), methanol (50 mL) and dried under vacuum to yield compound 683 (1.2 g, 2.48 mmol) as a tan solid. [M+H]⁺ calcd for C₂₉H₃₀N₃O₄: 484.22. Found: 484.01.

### Example 584

### N-(4-(4-(4-morpholinobenzamido)benzoyl)phenyl)-1H-pyrazole-3-carboxamide (Compound 684)

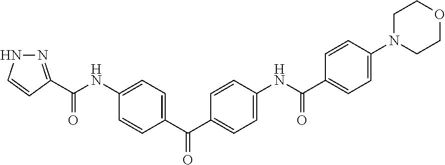 [SMILES: O=C(Nc1ccc(C(=O)c2ccc(NC(=O)c3cc[nH]n3)cc2)cc1)c1ccc(N2CCOCC2)cc1]

Compound 684 was prepared according to the procedure described in Scheme IV from 4,4′-diaminobenzophenone, 3-pyrazolecarboxylic and 4-morpholinobenzoic acids.

[M+H]⁺ calcd for C₂₈H₂₅N₅O₄: 496.05. Found: 496.02.

### Example 585

### N-(4-(4-(4-(4-Hydroxypiperidin-1-yl)benzamido)benzoyl)phenyl)-1-methyl-1H-pyrrole-2-carboxamide (Compound 685)

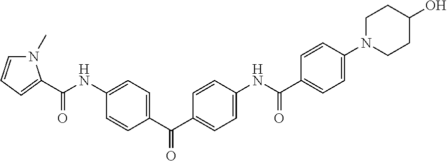 [SMILES: Cn1cccc1C(=O)Nc1ccc(C(=O)c2ccc(NC(=O)c3ccc(N4CCC(O)CC4)cc3)cc2)cc1]

Compound 685 was prepared according to the procedure described in Scheme IV from 4,4′-diaminobenzophenone, 1-methyl-2-pyrrolecarboxylic and 4-(4-hydroxypiperidin-1-yl)benzoic acids. [M+H]⁺ calcd for C₃₁H₃₀N₄O₄: 523.12. Found: 523.09.

### Example 586

### N-(4-(4-(4-(4-Hydroxypiperidin-1-yl)benzamido)benzoyl)phenyl)pyrimidine-5-carboxamide (Compound 686)

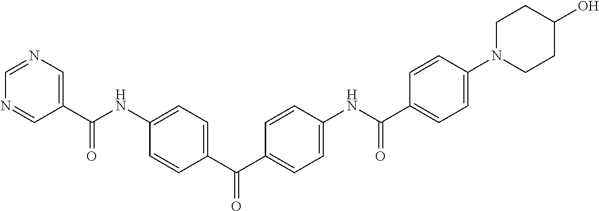 [SMILES: O=C(Nc1ccc(C(=O)c2ccc(NC(=O)c3cncnc3)cc2)cc1)c1ccc(N2CCC(O)CC2)cc1]

Compound 686 was prepared according to the procedure described in Scheme IV from 4,4′-diaminobenzophenone, pyrimidine-5-carboxylic and 4-(4-hydroxypiperidin-1-yl)benzoic acids. [M+H]⁺ calcd for C₃₀H₂₇N₅O₄: 522.09. Found: 522.08.

### Example 587

### N-Cyclopropyl-4-(4-(4-(4-hydroxypiperidin-1-yl)benzamido)benzoyl)benzamide (Compound 687)

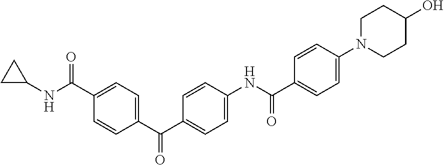 [SMILES: O=C(Nc1ccc(C(=O)c2ccc(C(=O)NC3CC3)cc2)cc1)c1ccc(N2CCC(O)CC2)cc1]

Compound 687 was prepared according to the procedure described in Scheme IV from N-cyclopropyl-4-(4-aminobenzoyl)benzamide and 4-(4-hydroxypiperidin-1-yl)benzoic acid. [M+H]⁺ calcd for C₂₉H₃₀N₃O₄: 484.22. Found: 484.08.

### Example 588

### N-(4-(4-(4-(4-Hydroxypiperidin-1-yl)benzamido)benzoyl)phenyl)-1H-indolyl-3-carboxamide (Compound 688)

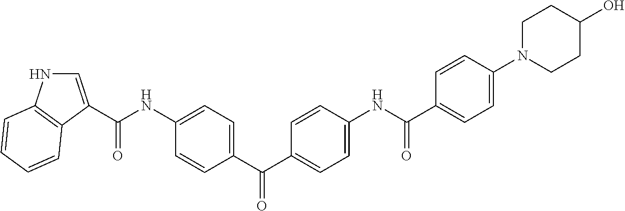 [SMILES: O=C(Nc1ccc(C(=O)c2ccc(NC(=O)c3c[nH]c4ccccc34)cc2)cc1)c1ccc(N2CCC(O)CC2)cc1]

Compound 688 was prepared according to the procedure described in Scheme IV from 4,4′-diaminobenzophenone, 3-indolecarboxylic and 4-(4-hydroxypiperidin-1-yl)benzoic acids. [M+H]⁺ calcd for C₃₄H₃₀N₄O₄: 559.15. Found: 559.14.

### Example 589

### N-(4-(4-(4-(4-Hydroxypiperidin-1-yl)benzamido)benzoyl)phenyl)-1H-indolyl-4-carboxamide (Compound 689)

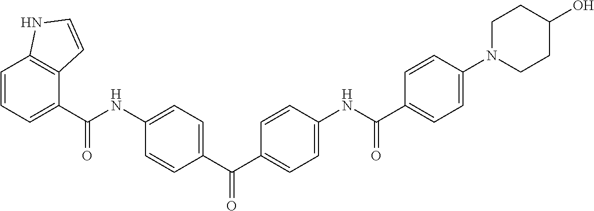 [SMILES: O=C(Nc1ccc(C(=O)c2ccc(NC(=O)c3cccc4[nH]ccc34)cc2)cc1)c1ccc(N2CCC(O)CC2)cc1]

Compound 689 was prepared according to the procedure described in Scheme IV from 4,4′-diaminobenzophenone, 4-indolecarboxylic and 4-(4-hydroxypiperidin-1-yl)benzoic acids. [M+H]⁺ calcd for C₃₄H₃₀N₄O₄: 559.15. Found: 559.14.

### Example 590

### N-(4-(4-(4-(4-Hydroxypiperidin-1-yl)benzamido)benzoyl)phenyl)-1H-indolyl-7-carboxamide (Compound 690)

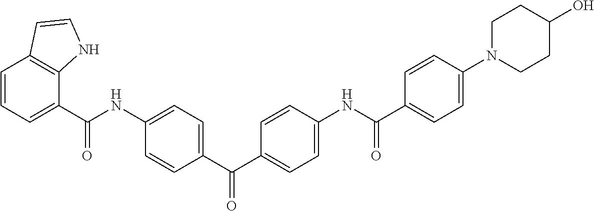 [SMILES: O=C(Nc1ccc(C(=O)c2ccc(NC(=O)c3cccc4cc[nH]c34)cc2)cc1)c1ccc(N2CCC(O)CC2)cc1]

Compound 690 was prepared according to the procedure described in Scheme IV from 4,4′-diaminobenzophenone, 7-indolecarboxylic and 4-(4-hydroxypiperidin-1-yl)benzoic acids. [M+H]⁺ calcd for C₃₄H₃₀N₄O₄: 559.15. Found: 559.07.

### Example 591

### N-(4-(4-(4-(4-hydroxypiperidin-1-yl)benzamido)benzoyl)phenyl)-4-hydroxypiperidine-1-carboxamide (Compound 691)

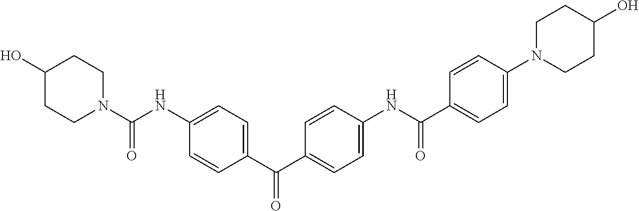 [SMILES: O=C(Nc1ccc(C(=O)c2ccc(NC(=O)N3CCC(O)CC3)cc2)cc1)c1ccc(N2CCC(O)CC2)cc1]

Compound 691 was prepared according to the procedure described in Scheme IV from 4,4′-diaminobenzophenone, 4-hydroxypiperidine, and 4-(4-hydroxypiperidin-1-yl)benzoic acid. [M+H]⁺ calcd for C₃₁H₃₅N₄O₅: 543.26. Found: 543.07.

### Example 592

### N-(4-(4-(4-(4-hydroxypiperidin-1-yl)benzamido)benzoyl)phenyl)-1H-pyrrolo[2,3-b]pyridine-5-carboxamide (Compound 692)

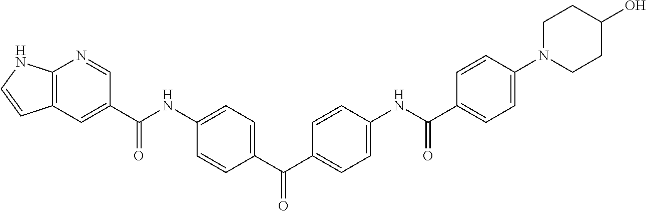 [SMILES: O=C(Nc1ccc(C(=O)c2ccc(NC(=O)c3cnc4[nH]ccc4c3)cc2)cc1)c1ccc(N2CCC(O)CC2)cc1]

Compound 692 was prepared according to the procedure described in Scheme IV from 4,4′-diaminobenzophenone, 1H-pyrrolo[2,3-b]pyridine-5-carboxylic and 4-(4-hydroxypiperidin-1-yl)benzoic acids. [M+H]⁺ calcd for C₃₃H₂₉N₅O₄: 560.14. Found: 560.08.

### Example 593

### N-(4-(4-(4-(4-hydroxypiperidin-1-yl)benzamido)benzoyl)phenyl)benzo[b]thiophene-2-carboxamide (Compound 693)

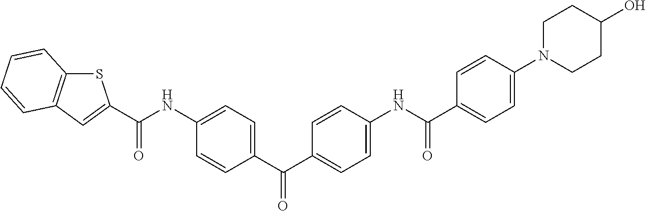 [SMILES: O=C(Nc1ccc(C(=O)c2ccc(NC(=O)c3cc4ccccc4s3)cc2)cc1)c1ccc(N2CCC(O)CC2)cc1]

Compound 693 was prepared according to the procedure described in Scheme IV from 4,4′-diaminobenzophenone, 2-benzothiophenecarboxylic and 4-(4-hydroxypiperidin-1-yl)benzoic acids. [M+H]⁺ calcd for C₃₄H₂₉N₃O₄S: 576.20. Found: 576.08.

### Example 594

### 4-Azido-N-(4-(4-(4-morpholinobenzamido)benzoyl)phenyl)benzamide (Compound 694)

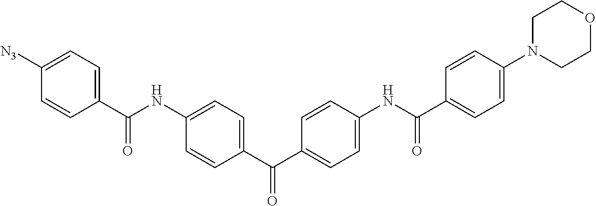 [SMILES: [N-]=[N+]=Nc1ccc(C(=O)Nc2ccc(C(=O)c3ccc(NC(=O)c4ccc(N5CCOCC5)cc4)cc3)cc2)cc1]

Compound 694 was prepared according to the procedure described in Scheme IV from 4,4′-diaminobenzophenone, 4-azidobenzoic and 4-morpholinobenzoic acids. ¹H NMR (500 MHz, DMSO-d₆) δ 10.56 (s, 1H), 10.28 (s, 1H), 8.04 (d, J=8.8 Hz, 2H), 7.97 (d, J=9.0 Hz, 2H), 7.96 (d, J=8.8 Hz, 2H), 7.91 (d, J=9.0 Hz, 2H), 7.76 (d, J=8.5 Hz, 2H), 7.74 (d, J=8.8 Hz, 2H), 7.28 (d, J=8.5 Hz, 2H), 7.03 (d, J=9.0 Hz, 2H), 3.75-3.73 (m, 4H), 3.27-3.25 (m, 4H).

### Example 595

### N-(4-(4-(4-(4-Hydroxypiperidin-1-yl)benzamido)benzoyl)phenyl)-4-methyl-1H-pyrrole-2-carboxamide (Compound 695)

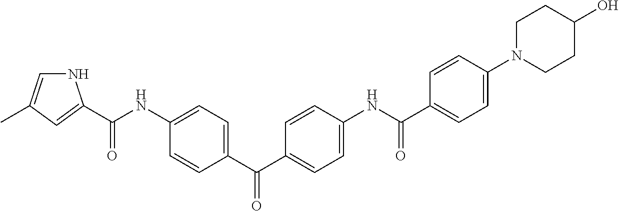 [SMILES: Cc1c[nH]c(C(=O)Nc2ccc(C(=O)c3ccc(NC(=O)c4ccc(N5CCC(O)CC5)cc4)cc3)cc2)c1]

Compound 695 was prepared according to the procedure described in Scheme IV from 4,4′-diaminobenzophenone, 4-methyl-2-pyrrolecarboxylic and 4-(4-hydroxypiperidin-1-yl)benzoic acids. [M+H]⁺ calcd for C₃₁H₃₀N₄O₄: 523.12. Found: 523.09.

### Example 596

### N-(4-(4-(4-(4-Hydroxypiperidin-1-yl)benzamido)benzoyl)phenyl)-1H-pyrrole-3-carboxamide (Compound 696)

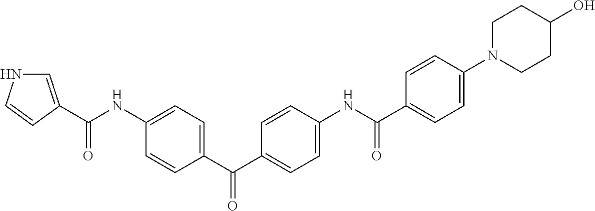 [SMILES: O=C(Nc1ccc(C(=O)c2ccc(NC(=O)c3cc[nH]c3)cc2)cc1)c1ccc(N2CCC(O)CC2)cc1]

Compound 696 was prepared according to the procedure described in Scheme IV from 4,4′-diaminobenzophenone, 3-pyrrolecarboxylic and 4-(4-hydroxypiperidin-1-yl)benzoic acids. [M+H]⁺ calcd for C₃₀H₂₈N₄O₄: 509.09. Found: 509.05.

### Example 597

### N-(4-(4-(4-(4-Hydroxypiperidin-1-yl)benzamido)benzoyl)phenyl)-1H-pyrrole-2-carboxamide (Compound 697)

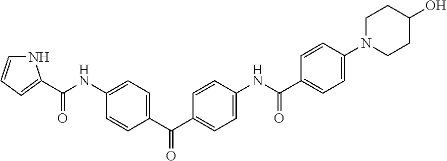 [SMILES: O=C(Nc1ccc(C(=O)c2ccc(NC(=O)c3ccc[nH]3)cc2)cc1)c1ccc(N2CCC(O)CC2)cc1]

Compound 697 was prepared according to the procedure described in Scheme IV from 4,4′-diaminobenzophenone, 2-pyrrolecarboxylic and 4-(4-hydroxypiperidin-1-yl)benzoic acids. [M+H]⁺ calcd for C₃₀H₂₈N₄O₄: 509.09. Found: 509.05.

### Example 598

### N-(4-(4-(4-(4-Hydroxypiperidin-1-yl)benzamido)benzoyl)phenyl)-4-(3-hydroxypropyl)piperazine-1-carboxamide (Compound 698)

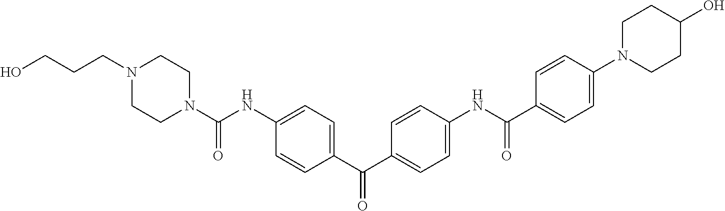 [SMILES: O=C(Nc1ccc(C(=O)c2ccc(NC(=O)N3CCN(CCCO)CC3)cc2)cc1)c1ccc(N2CCC(O)CC2)cc1]

Compound 698 was prepared according to the procedure described in Scheme IV from 4,4′-diaminobenzophenone, 4-(3-hydroxypropyl)piperazine, and 4-(4-hydroxypiperidin-1-yl)benzoic acid. [M+H]⁺ calcd for C₃₃H₄₀N₅O₅: 586.30. Found: 586.20.

### Example 599

### N-(4-(4-(4-(4-Hydroxypiperidin-1-yl)benzamido)benzoyl)phenyl)-4,5,6,7-tetrahydro-1H-indole-2-carboxamide (Compound 699)

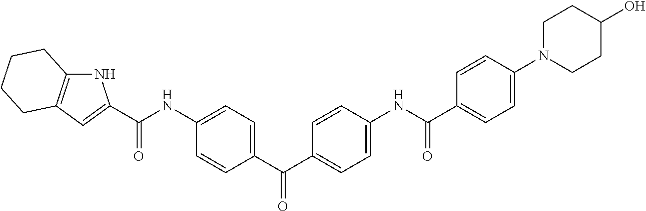 [SMILES: O=C(Nc1ccc(C(=O)c2ccc(NC(=O)c3cc4c([nH]3)CCCC4)cc2)cc1)c1ccc(N2CCC(O)CC2)cc1]

Compound 699 was prepared according to the procedure described in Scheme IV from 4,4′-diaminobenzophenone, 4,5,6,7-tetrahydroindole-2-carboxylic and 4-(4-hydroxypiperidin-1-yl)benzoic acids. [M+H]⁺ calcd for C₃₄H₃₄N₄O₄: 563.18. Found: 563.19.

### Example 600

### (±)-N-(4-(4-(4-(3-(Hydroxymethyl)piperidin-1-yl)benzamido)benzoyl)phenyl)-1H-pyrrole-2-carboxamide (Compound 700)

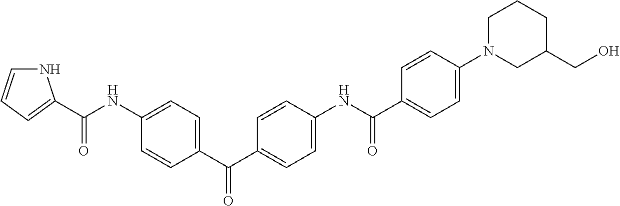 [SMILES: O=C(Nc1ccc(C(=O)c2ccc(NC(=O)c3ccc[nH]3)cc2)cc1)c1ccc(N2CCCC(CO)C2)cc1]

Compound 700 was prepared according to the procedure described in Scheme IV from 4,4′-diaminobenzophenone, 2-pyrrolecarboxylic and 4-(3-hydroxymethylpiperidin-1-yl)benzoic acids. ¹H NMR (400 MHz, DMSO-d₆) δ 10.25 (s, 1H), 10.07 (s, 1H), 7.98 (dd, J=9.0, 10 Hz, 4H), 7.91 (d, J=9.0 Hz, 2H), 7.77 (dd, J=1.6, 8.7 Hz, 4H), 7.16 (bm, 1H), 7.02 (s, 1H), 7.01 (d, J=9.0 Hz, 2H), 6.22 (m, 1H), 4.59 (t, J=5.3 Hz, 1H), 1H), 3.88 (m, 2H), 2.86 (t, J=12 Hz, 1H), 1.75 (m, 4H), 1.54 (m, 2H), 1.21 (m, 2H).

### Example 601

### N,N′-(Ethene-1,1-diylbis(4,1-phenylene))bis(4-(dimethylamino)benzamide) (Compound 701)

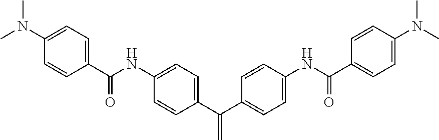

Compound 701 was prepared according to the procedure described in Scheme IV from 4,4′-(ethene-1,1-diyl)dianiline and 4-N,N-dimethylaminobenzoic acid. ¹H NMR (500 MHz, DMSO-d₆) δ 9.94 (s, 2H), 7.86 (d, J=8.8 Hz, 4H), 7.77 (d, J=8.5 Hz, 4H), 7.26 (d, J=8.3 Hz, 4H), 6.75 (d, J=8.8 Hz, 4H), 5.38 (s, 2H), 2.99 (s, 12H).

### Example 602

### N,N′-(Ethene-1,1-diylbis(4,1-phenylene))bis(1-methyl-1H-indole-5-carboxamide) (Compound 702)

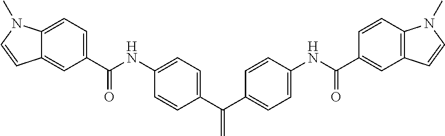 [SMILES: C=C(c1ccc(NC(=O)c2ccc3c(ccn3C)c2)cc1)c1ccc(NC(=O)c2ccc3c(ccn3C)c2)cc1]

Compound 702 was prepared according to the procedure described in Scheme IV from 4,4′-(ethene-1,1-diyl)dianiline and 1-methylindole-5-carboxylic acid. ¹H NMR (500 MHz, CDCl₃) δ 8.20 (s, 2H), 7.93 (s, 2H), 7.79 (dd, J=1.7, 8.5 Hz, 2H), 7.66 (dd, J=1.7, 6.8 Hz, 4H), 7.42 (d, J=2.3 Hz, 2H), 7.39 (d, J=8.5 Hz, 4H), 7.15 (d, J=2.3 Hz, 2H), 6.61 (d, J=2.9 Hz, 2H), 5.44 (s, 2H), 3.86 (s, 6H).

### Example 603

### N,N′-(((Methoxyimino)methylene)bis(4,1-phenylene))bis(1H-indole-5-carboxamide) (Compound 703)

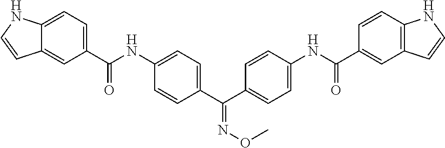 [SMILES: CON=C(c1ccc(NC(=O)c2ccc3[nH]ccc3c2)cc1)c1ccc(NC(=O)c2ccc3[nH]ccc3c2)cc1]

Compound 703 was prepared according to the procedure described in Scheme IV from 4,4′-diaminobenzophenone and 1-methylindole-5-carboxylic acid. [M+H]⁺ calcd for C₃₂H₂₅N₅O₃: 528.10. Found: 528.15.

### Example 604

### (Z/E)-N-(4-((4-(Cyclopropanecarboxamido)phenyl)(methoxyimino)methyl)phenyl)-1H-indole-5-carboxamide (Compound 704)

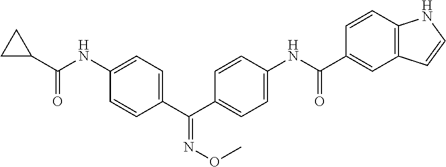 [SMILES: CO/N=C(\c1ccc(NC(=O)c2ccc3[nH]ccc3c2)cc1)c1ccc(NC(=O)C2CC2)cc1]

Compound 704 was prepared according to the procedure described in Scheme IV from 4,4′-diaminobenzophenone, cyclopropanecarboxylic, and indole-5-carboxylic acids.

[M+H]⁺ calcd for C₂₇H₂₄N₄O₃: 453.03. Found: 453.03.

### Example 605

### N-(4-(4-(1H-Pyrrole-2-carboxamido)benzoyl)phenyl)-1-(2-morpholinoethyl)-1H-indole-5-carboxamide (Compound 705)

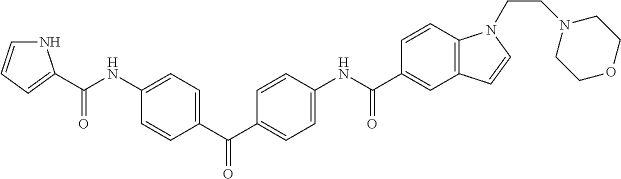 [SMILES: O=C(Nc1ccc(C(=O)c2ccc(NC(=O)c3ccc[nH]3)cc2)cc1)c1ccc2c(ccn2CCN2CCOCC2)c1]

Compound 705 was prepared according to the procedure described in Scheme IV from 4,4′-diaminobenzophenone, 2-pyrrolecarboxylic, and 1-(2-morpholinoethyl)indole-5-carboxylic acids. [M+H]⁺ calcd for C₃₃H₃₁N₅O₄: 562.16. Found: 562.11.

### Example 606

### (±)—N-(4-(4-(4-(2-(Hydroxymethyl)morpholino)benzamido)benzoyl)phenyl)-1H-pyrrole-2-carboxamide (Compound 706)

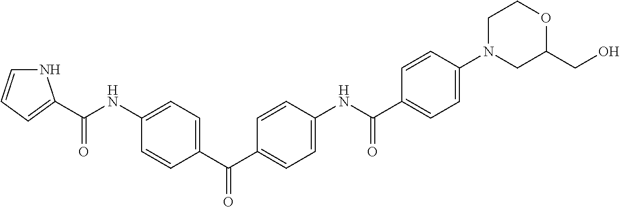 [SMILES: O=C(Nc1ccc(C(=O)c2ccc(NC(=O)c3ccc[nH]3)cc2)cc1)c1ccc(N2CCOC(CO)C2)cc1]

Compound 706 was prepared according to the procedure described in Scheme IV from 4,4′-diaminobenzophenone, 2-pyrrolecarboxylic, and 4-(2-hydroxymethyl)morpholino-4-)benzoic acids. [M+H]⁺ calcd for C₃₀H₂₈N₄O₅: 525.21. Found: 524.98.

### Example 607

### (±)-N-(4-(4-(4-(3-(Hydroxymethyl)piperidin-1-yl)benzamido)benzoyl)phenyl)-1 methyl-1H-pyrrole-2-carboxamide (Compound 707)

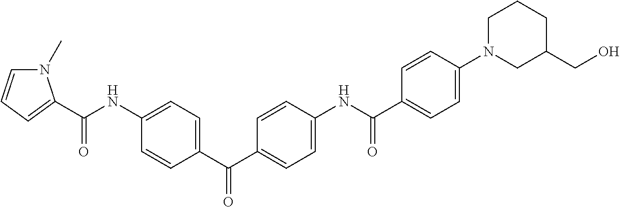 [SMILES: Cn1cccc1C(=O)Nc1ccc(C(=O)c2ccc(NC(=O)c3ccc(N4CCCC(CO)C4)cc3)cc2)cc1]

Compound 707 was prepared according to the procedure described in Scheme IV from 4,4′-diaminobenzophenone, 1-methyl-2-pyrrolecarboxylic, and 4-(3-hydroxymethylpiperidin-1-yl)benzoic acids. [M+H]⁺ calcd for C₃₂H₃₂N₄O₄: 537.24. Found: 537.06.

### Example 608

### (±)-N-(4-(4-(4-(3-Hydroxypyrrolidin-1-yl)benzamido)benzoyl)phenyl)-1-methyl-1H-pyrrole-2-carboxamide (Compound 708)

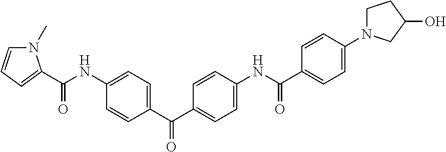 [SMILES: Cn1cccc1C(=O)Nc1ccc(C(=O)c2ccc(NC(=O)c3ccc(N4CCC(O)C4)cc3)cc2)cc1]

Compound 708 was prepared according to the procedure described in Scheme IV from 4,4′-diaminobenzophenone, 1-methyl-2-pyrrolecarboxylic, and 4-(3-hydroxypyrrolidin-1-yl)benzoic acids. [M+H]⁺ calcd for C₃₀H₂₈N₄O₄: 509.21. Found: 508.98.

### Example 609

### N-(4-(4-(4-(4-Hydroxypiperidin-1-yl)benzamido)benzoyl)phenyl)-4-morpholinecarboxamide (Compound 709)

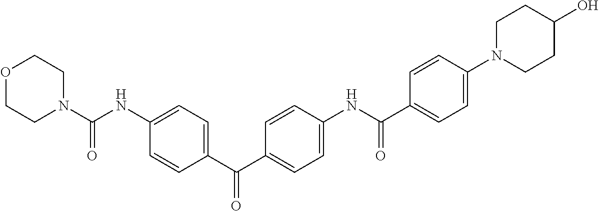 [SMILES: O=C(Nc1ccc(C(=O)c2ccc(NC(=O)N3CCOCC3)cc2)cc1)c1ccc(N2CCC(O)CC2)cc1]

Compound 709 was prepared according to the procedure described in Scheme IV from 4,4′-diaminobenzophenone, morpholine, and 4-(4-hydroxypiperidin-1-yl)benzoic acid. [M+H]⁺ calcd for C₃₀H₃₂N₄O₅: 529.12. Found: 529.03.

### Example 610

### (±)-N-(4-(4-(4-(2-(Hydroxymethyl)morpholino)benzamido)benzoyl)phenyl)-1-methyl-1H-pyrrole-2-carboxamide (Compound 710)

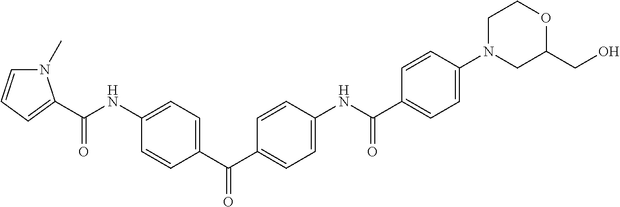 [SMILES: Cn1cccc1C(=O)Nc1ccc(C(=O)c2ccc(NC(=O)c3ccc(N4CCOC(CO)C4)cc3)cc2)cc1]

Compound 710 was prepared according to the procedure described in Scheme IV from 4,4′-diaminobenzophenone, 1-methyl-2-pyrrolecarboxylic, and 4-(2-hydroxymethyl)morpholino-4-)benzoic acids. [M+H]⁺ calcd for C₃₁H₃₀N₄O₅: 539.22. Found: 539.02.

### Example 611

### N-(4-(4-(4-(4-(Cyclopropanecarbonyl)piperazin-1-yl)benzamido)benzoyl)phenyl)-1-methyl-1H-pyrrole-2-carboxamide (Compound 711)

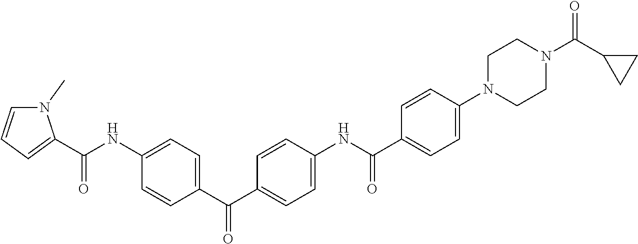 [SMILES: Cn1cccc1C(=O)Nc1ccc(C(=O)c2ccc(NC(=O)c3ccc(N4CCN(C(=O)C5CC5)CC4)cc3)cc2)cc1]

Compound 711 was prepared according to the procedure described in Scheme IV from 4,4′-diaminobenzophenone, 1-methyl-2-pyrrolecarboxylic, and 4-(4-cyclopropanecarbonyl)piperazinobenzoic acids. [M+H]⁺ calcd for C₃₄H₃₃N₅O₄: 576.25. Found: 576.08.

### Example 612

### (R)—N-(4-(4-(4-(3-Hydroxypyrrolidin-1-yl)benzamido)benzoyl)phenyl)-1-methyl-1H-pyrrole-2-carboxamide (Compound 712)

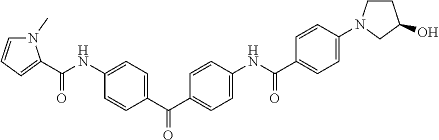 [SMILES: Cn1cccc1C(=O)Nc1ccc(C(=O)c2ccc(NC(=O)c3ccc(N4CC[C@@H](O)C4)cc3)cc2)cc1]

Compound 712 was prepared according to the procedure described in Scheme IV from 4,4′-diaminobenzophenone, 1-methyl-2-pyrrolecarboxylic, and (R)-4-(3-hydroxypyrrolidin-1-yl)benzoic acids. [M+H]⁺ calcd for C₃₀H₂₈N₄O₄: 509.21. Found: 508.98.

### Example 613

### (S)—N-(4-(4-(4-(3-Hydroxypyrrolidin-1-yl)benzamido)benzoyl)phenyl)-1-methyl-1H-pyrrole-2-carboxamide (Compound 713)

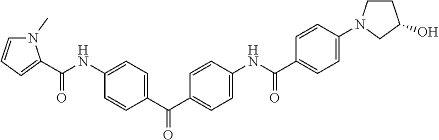 [SMILES: Cn1cccc1C(=O)Nc1ccc(C(=O)c2ccc(NC(=O)c3ccc(N4CC[C@H](O)C4)cc3)cc2)cc1]

Compound 713 was prepared according to the procedure described in Scheme IV from 4,4′-diaminobenzophenone, 1-methyl-2-pyrrolecarboxylic, and (S)-4-(3-hydroxypyrrolidin-1-yl)benzoic acids. ¹H NMR (400 MHz, Acetone-d₆) δ 9.49 (s, 1H), 9.38 (s, 1H), 8.02 (d, J=8.8 Hz, 2H), 7.95 (t, J=9 Hz, 3H), 7.78 (d, J=7 Hz, 3H), 7.03 (dd, J=2, 4 Hz, 1H), 6.97 (t, J=2 Hz, 1H), 6.61 (d, J=9 Hz, 2H), 6.11 (dd, J=2.5, 4 Hz, 1H), 4.60 (m, 1H), 3.99 (s, 3H), 3.59-3.42 (m, 3H), 3.31 (m, 1H), 2.20-2.14 (m, 3H).

### Example 614

### 4-Azido-N-(4-(4-(4-(dimethylamino)benzamido)benzoyl)phenyl)benzamide (Compound 714)

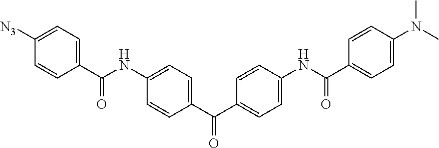 [SMILES: CN(C)c1ccc(C(=O)Nc2ccc(C(=O)c3ccc(NC(=O)c4ccc(N=[N+]=[N-])cc4)cc3)cc2)cc1]

Compound 714 was prepared according to the procedure described in Scheme IV from 4,4′-diaminobenzophenone, 4-azidobenzoic, and 4-dimtheylaminobenzoic acids. [M+H]⁺ calcd for C₂₉H₂₄N₆O₃: 505.19. Found 504.93.

### Example 615

### N-(4-(4-(3-Fluoro-4-(4-hydroxypiperidin-1-yl)benzamido)benzoyl)phenyl)-1-methyl-1H-pyrrole-2-carboxamide (Compound 715)

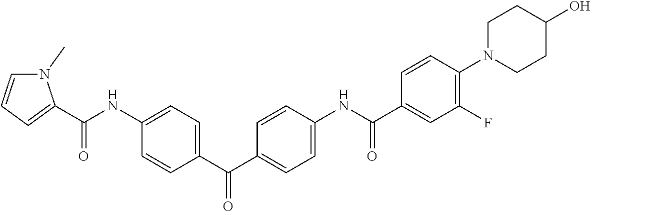 [SMILES: Cn1cccc1C(=O)Nc1ccc(C(=O)c2ccc(NC(=O)c3ccc(N4CCC(O)CC4)c(F)c3)cc2)cc1]

Compound 715 was prepared according to the procedure described in Scheme IV from 4,4′-diaminobenzophenone, 1-methyl-2-pyrrolecarboxylic, and 4-(4-hydroxypiperidin-1-yl)-3-fluorobenzoic acids. [M+H]⁺ calcd for C₃₁H₂₉FN₄O₄: 541.22. Found: 541.05.

### Example 616

### (E/Z)-N-(4-((4-(4-(4-Hydroxypiperidin-1-yl)benzamido)phenyl)(methoxyimino)methyl)phenyl)-1-methyl-1H-pyrrole-2-carboxamide (Compound 716)

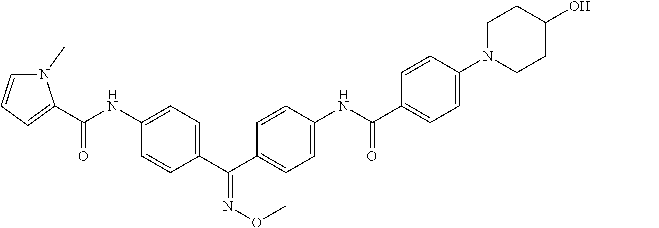 [SMILES: CO/N=C(\c1ccc(NC(=O)c2ccc(N3CCC(O)CC3)cc2)cc1)c1ccc(NC(=O)c2cccn2C)cc1]

Compound 716 was prepared according to the procedure described in Scheme IV from 4,4′-diaminobenzophenone, 1-methyl-2-pyrrolecarboxylic, and 4-(4-hydroxypiperidin-1-yl)benzoic acids. [M+H]⁺ calcd for C₃₂H₃₃N₅O₄: 552.16. Found: 552.05.

### Example 617

### N-(4-(4-(4-(4-Hydroxypiperidin-1-yl)benzamido)benzoyl)phenyl)-1H-pyrazole-5-carboxamide (Compound 717)

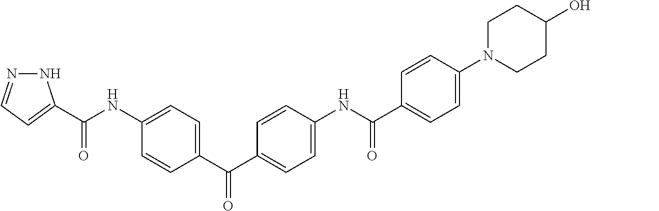 [SMILES: O=C(Nc1ccc(C(=O)c2ccc(NC(=O)c3ccn[nH]3)cc2)cc1)c1ccc(N2CCC(O)CC2)cc1]

Compound 717 was prepared according to the procedure described in Scheme IV from 4,4′-diaminobenzophenone, 5-pyrazolecarboxylic, and 4-(4-hydroxypiperidin-1-yl)benzoic acids. [M+H]⁺ calcd for C₂₉H₂₇N₅O₄: 510.18. Found: 509.99.

### Example 618

### N-(4-(4-(3-Trifluoromethyl-4-(4-hydroxypiperidin-1-yl)benzamido)benzoyl)phenyl)-1-methyl-1H-pyrrole-2-carboxamide (Compound 718)

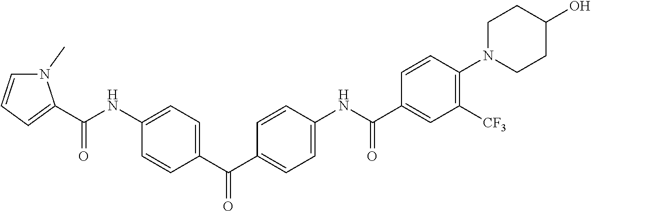 [SMILES: Cn1cccc1C(=O)Nc1ccc(C(=O)c2ccc(NC(=O)c3ccc(N4CCC(O)CC4)c(C(F)(F)F)c3)cc2)cc1]

Compound 718 was prepared according to the procedure described in Scheme IV from 4,4′-diaminobenzophenone, 1-methyl-2-pyrrolecarboxylic, and 4-(4-hydroxypiperidin-1-yl)-3-trifluoromethylbenzoic acids. [M+H]⁺ calcd for C₃₂H₃₀F₃N₄O₄: 591.22. Found: 591.07.

### Example 619

### N-(4-(4-(4-(4-Hydroxypiperidin-1-yl)benzamido)benzoyl)phenyl)-1H-pyrazole-4-carboxamide (Compound 719)

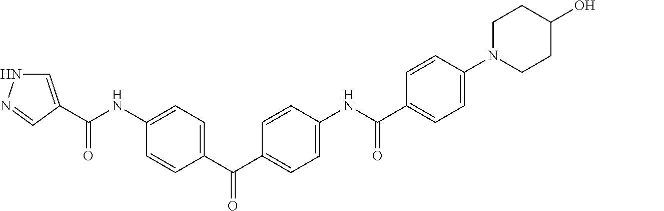 [SMILES: O=C(Nc1ccc(C(=O)c2ccc(NC(=O)c3cn[nH]c3)cc2)cc1)c1ccc(N2CCC(O)CC2)cc1]

Compound 719 was prepared according to the procedure described in Scheme IV from 4,4′-diaminobenzophenone, 4-pyrazolecarboxylic, and 4-(4-hydroxypiperidin-1-yl)benzoic acids. [M+H]⁺ calcd for C₂₉H₂₇N₅O₄: 510.18. Found: 509.99.

### Example 620

### N-(4-(4-(4-(4-Hydroxypiperidin-1-yl)benzamido)benzoyl)phenyl)-1-methyl-1H-pyrazole-5-carboxamide (Compound 720)

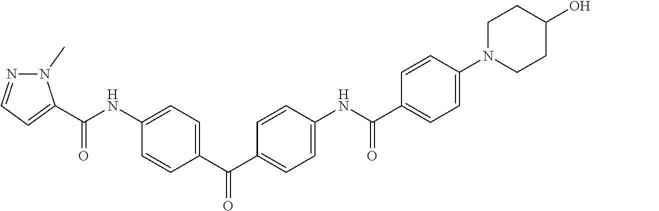 [SMILES: Cn1nccc1C(=O)Nc1ccc(C(=O)c2ccc(NC(=O)c3ccc(N4CCC(O)CC4)cc3)cc2)cc1]

Compound 720 was prepared according to the procedure described in Scheme IV from 4,4′-diaminobenzophenone, 1-methyl-5-pyrazolecarboxylic, and 4-(4-hydroxypiperidin-1-yl)benzoic acids. [M+H]⁺ calcd for C₃₀H₂₉N₅O₄: 524.11. Found: 524.03.

### Example 621

### N-(4-(4-(2-Methyl-4-(4-hydroxypiperidin-1-yl)benzamido)benzoyl)phenyl)-1-methyl-1H-pyrrole-2-carboxamide (Compound 721)

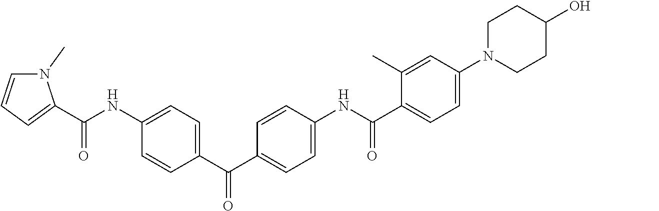 [SMILES: Cc1cc(N2CCC(O)CC2)ccc1C(=O)Nc1ccc(C(=O)c2ccc(NC(=O)c3cccn3C)cc2)cc1]

Compound 721 was prepared according to the procedure described in Scheme IV from 4,4′-diaminobenzophenone, 1-methyl-2-pyrrolecarboxylic, and 4-(4-hydroxypiperidin-1-yl)-2-methylbenzoic acids. [M+H]⁺ calcd for C₃₂H₃₃N₄O₄: 537.25. Found: 537.06.

### Example 622

### N-(4-(4-(3-Methoxy-4-(4-hydroxypiperidin-1-yl)benzamido)benzoyl)phenyl)-1-methyl-1H-pyrrole-2-carboxamide (Compound 722)

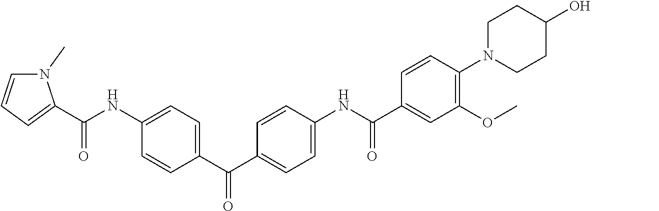 [SMILES: COc1cc(C(=O)Nc2ccc(C(=O)c3ccc(NC(=O)c4cccn4C)cc3)cc2)ccc1N1CCC(O)CC1]

Compound 722 was prepared according to the procedure described in Scheme IV from 4,4′-diaminobenzophenone, 1-methyl-2-pyrrolecarboxylic, and 4-(4-hydroxypiperidin-1-yl)-3-methoxybenzoic acids. [M+H]⁺ calcd for C₃₂H₃₃N4O₅: 553.25. Found: 553.06.

### Example 623

### 5-Chloro-N-(4-(4-(4-(4-hydroxypiperidin-1-yl)benzamido)benzoyl)phenyl)-1-isopropyl-1H-pyrrole-2-carboxamide (Compound 723)

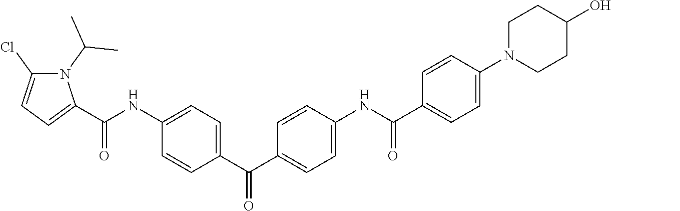 [SMILES: CC(C)n1c(Cl)ccc1C(=O)Nc1ccc(C(=O)c2ccc(NC(=O)c3ccc(N4CCC(O)CC4)cc3)cc2)cc1]

Compound 723 was prepared according to the procedure described in Scheme IV from 4,4′-diaminobenzophenone, 5-chloro-1-isopropyl-2-pyrrolecarboxylic, and 4-(4-hydroxypiperidin-1-yl)-3-trifluoromethylbenzoic acids. [M+H]⁺ calcd for C₃₃H₃₃ClN₄O₄: 585.22. Found: 585.06.

### Example 624

### N-(4-(4-(4-(4-Hydroxypiperidin-1-yl)benzamido)benzoyl)phenyl)-1-isopropyl-1H-pyrrole-2-carboxamide (Compound 724)

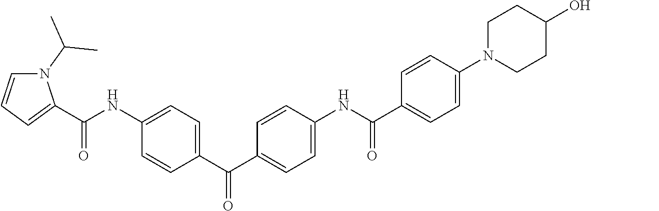 [SMILES: CC(C)n1cccc1C(=O)Nc1ccc(C(=O)c2ccc(NC(=O)c3ccc(N4CCC(O)CC4)cc3)cc2)cc1]

Compound 724 was prepared according to the procedure described in Scheme IV from 4,4′-diaminobenzophenone, 1-isopropyl-2-pyrrolecarboxylic, and 4-(4-hydroxypiperidin-1-yl)-3-trifluoromethylbenzoic acids. ¹H NMR (400 MHz, CD₃OD) δ 7.93-7.81 (m, 10H), 7.22 (dd, J=1.7, 2.7 Hz, 1H), 7.05 (d, J=9 Hz, 2H), 7.00 (dd, J=1.7, 4 Hz, 1H), 6.22 (dd, J=2.7, 4 Hz, 1H), 5.51 (s, 1H), 3.84 (m, 3H), 3.10 (m, 2H), 1.9 (m, 2H), 1.64 (m, 2H), 1.50 (s, 3H), 1.48 (s, 3H).

### Example 625

### N-(4-(4-(4-(4-Hydroxypiperidin-1-yl)benzamido)benzoyl)phenyl)-1-methyl-1H-pyrazole-4-carboxamide (Compound 725)

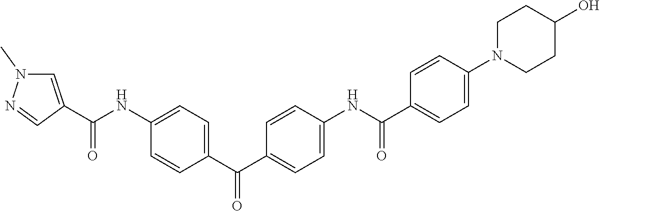 [SMILES: Cn1cc(C(=O)Nc2ccc(C(=O)c3ccc(NC(=O)c4ccc(N5CCC(O)CC5)cc4)cc3)cc2)cn1]

Compound 725 was prepared according to the procedure described in Scheme IV from 4,4′-diaminobenzophenone, 1-methyl-4-pyrazolecarboxylic, and 4-(4-hydroxypiperidin-1-yl)benzoic acids. [M+H]⁺ calcd for C₃₀H₂₉N₅O₄: 524.11. Found: 524.03.

### Example 626

### Methyl 5-((4-(4-(4-(4-hydroxypiperidin-1-yl)benzamido)benzoyl)phenyl)carbamoyl)-1H-pyrrole-2-carboxylate (Compound 726)

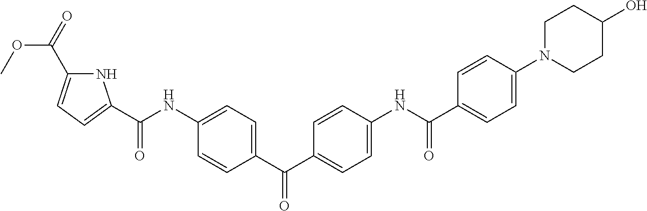 [SMILES: COC(=O)c1ccc(C(=O)Nc2ccc(C(=O)c3ccc(NC(=O)c4ccc(N5CCC(O)CC5)cc4)cc3)cc2)[nH]1]

Compound 726 was prepared according to the procedure described in Scheme IV from 4,4′-diaminobenzophenone, 1-isopropyl-2-pyrrolecarboxylic, and 4-(4-hydroxypiperidin-1-yl)-3-trifluoromethylbenzoic acids. [M+H]⁺ calcd for C₃₂H₃₀N₄O₆: 567.13. Found: 567.04.

### Example 627

### N-(4-(4-(4-(4-Hydroxypiperidin-1-yl)benzamido)benzoyl)phenyl)-1-methyl-1H-pyrazole-3-carboxamide (Compound 727)

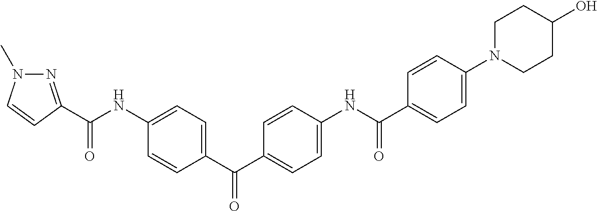 [SMILES: Cn1ccc(C(=O)Nc2ccc(C(=O)c3ccc(NC(=O)c4ccc(N5CCC(O)CC5)cc4)cc3)cc2)n1]

Compound 727 was prepared according to the procedure described in Scheme IV from 4,4′-diaminobenzophenone, 1-methyl-3-pyrazolecarboxylic, and 4-(4-hydroxypiperidin-1-yl)benzoic acids. [M+H]⁺ calcd for C₃₀H₂₉N₅O₄: 524.11. Found: 524.03.

### Example 628

### N-(4-(4-(3-Methyl-4-(4-hydroxypiperidin-1-yl)benzamido)benzoyl)phenyl)-1-methyl-1H-pyrrole-2-carboxamide (Compound 728)

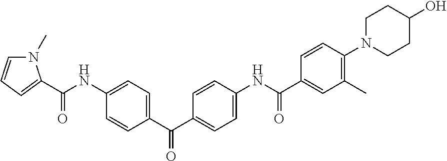 [SMILES: Cc1cc(C(=O)Nc2ccc(C(=O)c3ccc(NC(=O)c4cccn4C)cc3)cc2)ccc1N1CCC(O)CC1]

Compound 728 was prepared according to the procedure described in Scheme IV from 4,4′-diaminobenzophenone, 1-methyl-2-pyrrolecarboxylic, and 4-(4-hydroxypiperidin-1-yl)-3-methylbenzoic acids. [M+H]⁺ calcd for C₃₂H₃₃N₄O₄: 537.25. Found: 537.11.

### Example 629

### N-(4-(4-(4-(4-Hydroxypiperidin-1-yl)benzamido)benzoyl)phenyl)-1-vinyl-4,5,6,7-tetrahydro-1H-indole-2-carboxamide (Compound 729)

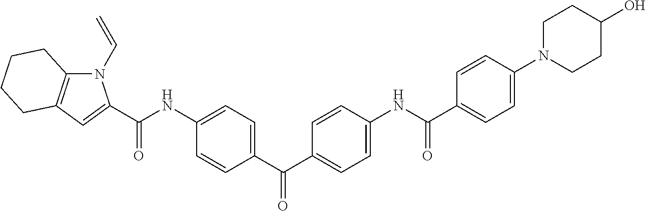 [SMILES: C=Cn1c(C(=O)Nc2ccc(C(=O)c3ccc(NC(=O)c4ccc(N5CCC(O)CC5)cc4)cc3)cc2)cc2c1CCCC2]

Compound 729 was prepared according to the procedure described in Scheme IV from 4,4′-diaminobenzophenone, 1-vinyl-4,5,6,7-tetrahydroindole-2-carboxylic, and 4-(4-hydroxypiperidin-1-yl)benzoic acids. [M+H]⁺ calcd for C₃₆H₃₆N₄O₄: 589.27. Found: 589.12.

### Example 630

### N-(4-(4-(2-Chloro-4-(4-hydroxypiperidin-1-yl)benzamido)benzoyl)phenyl)-1-methyl-1H-pyrrole-2-carboxamide (Compound 730)

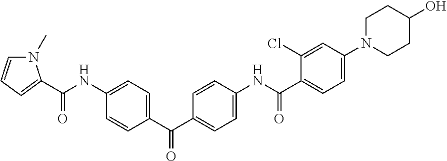 [SMILES: Cn1cccc1C(=O)Nc1ccc(C(=O)c2ccc(NC(=O)c3ccc(N4CCC(O)CC4)cc3Cl)cc2)cc1]

Compound 730 was prepared according to the procedure described in Scheme IV from 4,4′-diaminobenzophenone, 1-methyl-2-pyrrolecarboxylic, and 4-(4-hydroxypiperidin-1-yl)-2-chlorobenzoic acids. [M+H]⁺ calcd for C₃₁H₃₀ClN₄O₄: 557.20. Found: 556.98.

### Example 631

### N-(4-(4-(4-(4-Hydroxypiperidin-1-yl)benzamido)benzoyl)phenyl)-1-methyl-1H-pyrrole-3-carboxamide (Compound 731)

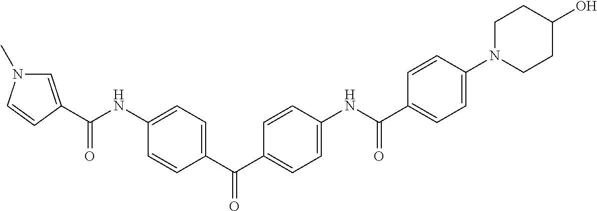 [SMILES: Cn1ccc(C(=O)Nc2ccc(C(=O)c3ccc(NC(=O)c4ccc(N5CCC(O)CC5)cc4)cc3)cc2)c1]

Compound 731 was prepared according to the procedure described in Scheme IV from 4,4′-diaminobenzophenone, 1-methyl-3-pyrrolecarboxylic, and 4-(4-hydroxypiperidin-1-yl)benzoic acids. [M+H]⁺ calcd for C₃₁H₃₀N₄O₄: 523.12. Found: 522.95.

### Example 632

### N-(4-(4-(2-Chloro-4-(4-hydroxypiperidin-1-yl)benzamido)benzoyl)phenyl)-1-ethyl-1H-pyrrole-2-carboxamide (Compound 732)

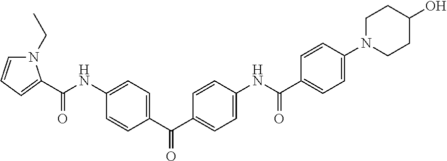 [SMILES: CCn1cccc1C(=O)Nc1ccc(C(=O)c2ccc(NC(=O)c3ccc(N4CCC(O)CC4)cc3)cc2)cc1]

Compound 732 was prepared according to the procedure described in Scheme IV from 4,4′-diaminobenzophenone, 1-ethyl-2-pyrrolecarboxylic, and 4-(4-hydroxypiperidin-1-yl)benzoic acids. ¹H NMR (400 MHz, CD₃OD) δ 7.89-7.79 (m, 10H), 7.02 (m, 4H), 6.15 (m, 1H), 4.41 (q, J=7.5 Hz, 2H), 3.82 (m, 3H), 3.07 (dt, J=3, 13 Hz, 2H), 1.96 (m, 2H), 1.60 (m, 2H), 1.39 (t, J=7.1 Hz, 3H).

### Example 633

### N-(4-(4-(4-(4-Hydroxypiperidin-1-yl)benzamido)benzoyl)phenyl)-4-oxo-4,5,6,7-tetrahydro-1H-indole-3-carboxamide (Compound 733)

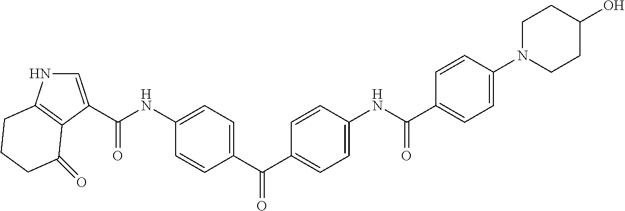 [SMILES: O=C(Nc1ccc(C(=O)c2ccc(NC(=O)c3c[nH]c4c3C(=O)CCC4)cc2)cc1)c1ccc(N2CCC(O)CC2)cc1]

Compound 733 was prepared according to the procedure described in Scheme IV from 4,4′-diaminobenzophenone, 4-oxo-4,5,6,7-tetrahydroindole-3-carboxylic, and 4-(4-hydroxypiperidin-1-yl)benzoic acids. ¹H NMR (500 MHz, DMSO-d₆) δ 12.91 (s, 1H), 12.20 (s, 1H), 10.24 (s, 1H), 7.98 (d, J=14 Hz, 2H), 7.90 (q, J=2, 14 Hz, 4H), 7.79 (m, 4H), 7.62 (s, 1H), 7.03 (d, J=10 Hz, 2H), 4.72 (d, J=8 Hz, 1H), 3.73 (m, 3H), 3.06 (m, 2H), 2.89 (t, J=8, 10 Hz, 2H), 2.61 (m, 2H), 2.11 (m, 2H), 1.82 (m, 2H), 1.47 (m, 2H).

### Example 634

### N-(4-(4-(4-(4-Hydroxypiperidin-1-yl)benzamido)benzoyl)phenyl)-2,5-dimethyl-1H-pyrrole-3-carboxamide (Compound 734)

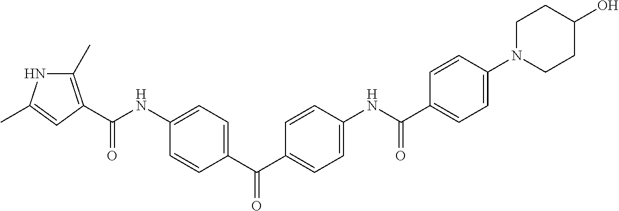 [SMILES: Cc1cc(C(=O)Nc2ccc(C(=O)c3ccc(NC(=O)c4ccc(N5CCC(O)CC5)cc4)cc3)cc2)c(C)[nH]1]

Compound 734 was prepared according to the procedure described in Scheme IV from 4,4′-diaminobenzophenone, 2,5-dimethyl-3-pyrrolecarboxylic, and 4-(4-hydroxypiperidin-1-yl)benzoic acids. ¹H NMR (500 MHz, DMSO-d₆) δ 10.99 (s, 1H), 10.23 (s, 1H), 9.52 (s, 1H), 7.98 (dd, J=12, 12 Hz, 4H), 7.89 (d, J=8 Hz, 2H), 7.72 (dd, J=8, 12 Hz, 4H), 7.02 (d, J=10 Hz, 2H), 6.40 (s, 1H), 4.71 (d, J=3 Hz, 1H), 3.72 (m, 3H), 3.05 (m, 2H), 1.83 (m, 2H), 1.45 (m, 2H).

### Example 635

### N-(4-(4-(4-(4-Hydroxypiperidin-1-yl)benzamido)benzoyl)phenyl)-1,2,5-trimethyl-1H-pyrrole-3-carboxamide (Compound 735)

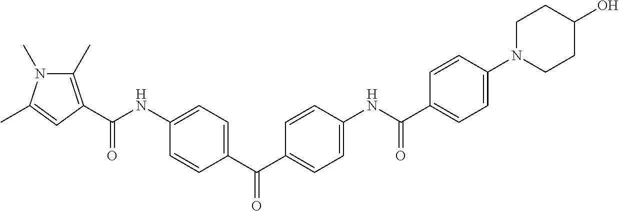 [SMILES: Cc1cc(C(=O)Nc2ccc(C(=O)c3ccc(NC(=O)c4ccc(N5CCC(O)CC5)cc4)cc3)cc2)c(C)n1C]

Compound 735 was prepared according to the procedure described in Scheme IV from 4,4′-diaminobenzophenone, 1,2,5-trimethyl-3-pyrrolecarboxylic, and 4-(4-hydroxypiperidin-1-yl)benzoic acids. ¹H NMR (500 MHz, DMSO-d₆) δ 10.24 (s, 1H), 9.57 (s, 1H), 7.98 (dd, J=10, 16 Hz, 4H), 7.89 (d, J=8 Hz, 2H), 7.72 (dd, J=10, 12 Hz, 4H), 7.02 (d, J=8 Hz, 2H), 6.49 (s, 1H), 4.71 (d, J=4 Hz, 1H), 3.72 (m, 3H), 3.41 (s, 3H), 3.06 (m, 2H), 1.86 (m, 2H), 1.48 (m, 2H).

### Example 636

### N-(4-(4-(2-Chloro-4-(4-hydroxypiperidin-1-yl)benzamido)benzoyl)phenyl)-1-cyanomethyl-1H-pyrrole-2-carboxamide (Compound 736)

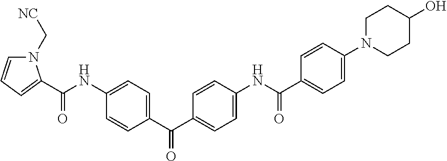 [SMILES: N#CCn1cccc1C(=O)Nc1ccc(C(=O)c2ccc(NC(=O)c3ccc(N4CCC(O)CC4)cc3)cc2)cc1]

Compound 736 was prepared according to the procedure described in Scheme IV from 4,4′-diaminobenzophenone, 1-cyanomethyl-2-pyrrolecarboxylic, and 4-(4-hydroxypiperidin-1-yl)benzoic acids. [M+H]⁺ calcd for C₃₂H₂₉N₅O₄: 548.22. Found: 548.07.

### Example 637

### N-(4-(4-(4-(4-(3-Hydroxypropyl)piperazin-1-yl)benzamido)benzoyl)phenyl)-1-methyl-1H-pyrrole-2-carboxamide (Compound 737)

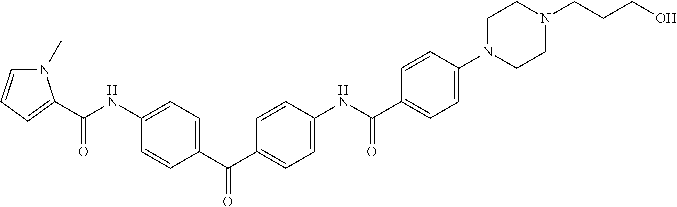 [SMILES: Cn1cccc1C(=O)Nc1ccc(C(=O)c2ccc(NC(=O)c3ccc(N4CCN(CCCO)CC4)cc3)cc2)cc1]

Compound 737 was prepared according to the procedure described in Scheme IV from 4,4′-diaminobenzophenone, 1-ethyl-2-pyrrolecarboxylic, and 4-(4-(3-hydroxypropyl)piperazin-1-yl)benzoic acids. [M+H]⁺ calcd for C₃₃H₃₆N₅O₄: 566.28. Found: 566.12.

### Example 638

### N-(4-(4-(2-Chloro-4-(4-hydroxypiperidin-1-yl)benzamido)benzoyl)phenyl)-1-vinyl-1H-pyrrole-2-carboxamide (Compound 738)

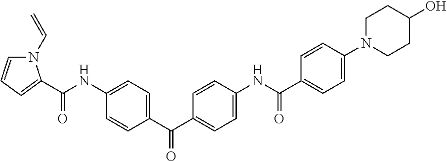 [SMILES: C=Cn1cccc1C(=O)Nc1ccc(C(=O)c2ccc(NC(=O)c3ccc(N4CCC(O)CC4)cc3)cc2)cc1]

Compound 738 was prepared according to the procedure described in Scheme IV from 4,4′-diaminobenzophenone, 1-vinyl-2-pyrrolecarboxylic, and 4-(4-hydroxypiperidin-1-yl)benzoic acids. ¹H NMR (500 MHz, DMSO-d₆) δ 10.38 (s, 1H), 10.35 (s, 1H), 7.99 (d, J=8 Hz, 2H), 7.90 (m, 4H), 7.77 (d, J=10 Hz, 4H), 7.63 (s, 1H), 7.20 (s, 1H), 7.02 (d, J=10 Hz, 2H), 6.34 (s, 1H), 5.42 (d, J=12 Hz, 1H), 4.88 (d, J=8 Hz, 1H), 4.71 (d, J=4 Hz, 1H), 3.81 (m, 3H), 3.05 (m, 2H), 1.83 (m, 2H), 1.47 (m, 2H).

### Example 639

### N-(4-((4-(4-(4-Hydroxypiperidin-1-yl)benzamido)phenyl)amino)phenyl)pyrimidine-5-carboxamide (Compound 739)

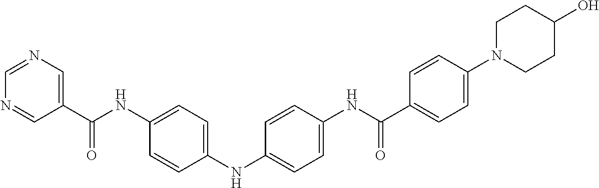 [SMILES: O=C(Nc1ccc(Nc2ccc(NC(=O)c3cncnc3)cc2)cc1)c1ccc(N2CCC(O)CC2)cc1]

Compound 739 was prepared according to the general procedure described in Scheme IV. Preparation of N-(4-nitrophenyl)benzene-1,4-diamine: Benzene-1,4-diamine (5.4 g, 50 mmol) and 1-fluoro-4-nitrobenzene (5.3 mL, 50 mmol) were dissolved in 75 mL dimethylsulfoxide and potassium carbonate (13.8 g, 100 mmol) was added. The reaction mixture was heated in an oil bath at 90° C. and stirred overnight under nitrogen atmosphere. The reaction mixture was cooled to room temperature and added to 250 mL water in a slow stream and stirred till a solid was precipitated out. The reaction mixture was filtered out and the resulting dark brown solid was washed with plenty of water. Flash column chromatography on silica gel eluted with 20% to 40% Ethyl acetate in hexanes provided the title compound (6.1 g, 53% yield).

Preparation of 4-(4-hydroxypiperidin-1-yl)-N-(4-((4-nitrophenyl)amino)phenyl)-benzamide: N-4(-4-nitrophenyl)benzene-1,4-diamine (1.15 g, 5.0 mmol) and 4-(4-hydroxypiperidine-1-yl)benzoic acid (1.2 g, 5.5 mmol) were dissolved in 20 mL pyridine and EDCI (1.2 g, 6.0 mmole) added and stirred at room temperature under nitrogen atmosphere overnight. Water (80 mL) was added and the mixture was stirred for an extra 15 minutes till a solid was precipitated out. The reaction mixture was filtered and the resulting red-color solid was washed with water, ethyl acetate, and hexanes, then dried to give the title compound (1.6 g, 80% yield.) This compound was used for next step without any further purification.

Preparation of Compound 739: 4-(4-Hydroxypiperidin-1-yl)-N-(4-((4-nitrophenyl)amino)phenyl)benzamide (1.8 g, 4.2 mmol) was hydrogenated using palladium hydroxide (1.6 g) in absolute ethanol (75 mL). The reaction mixture was stirred under hydrogen gas balloon overnight. It was filtered through celite and evaporated to dryness to give 4-(4-hydroxypiperidin-1-yl)-N-(4-((4-nitrophenyl)amino)phenyl)benzamide over 98% pure (1.6 g, 95% yield). Part of the product (22 mg, 0.05 mmol) and pyrimidine-4-carboxylic acid (8 mg, 0.06 mmole) were dissolved in 1 mL pyridine and was added EDCI (19 mg, 0.1 mmol). The reaction mixture was stirred at room temperature under nitrogen atmosphere overnight. Water (5 mL) was added and the mixture was stirred for an extra 15 minutes till a solid was precipitated out. The reaction mixture was filtered and the resulting red-color solid was washed with water, ethyl acetate, hexanes, and then dried to give crude compound 739 (20 mg 78% yield). Further purification was carried on using prep HPLC for the final sample. [M+H]⁺ calcd for C₂₉H₂₈N₆O₃: 509.22. Found 508.98.

### Example 640

### N-(4-((4-(4-(4-Hydroxypiperidin-1-yl)benzamido)phenyl)amino)phenyl)nicotinamide (Compound 740)

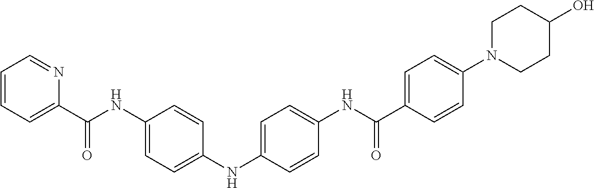 [SMILES: O=C(Nc1ccc(Nc2ccc(NC(=O)c3ccccn3)cc2)cc1)c1ccc(N2CCC(O)CC2)cc1]

Compound 740 was prepared according to the procedure described in Scheme IV from 4,4′-diaminodiphenylamine, nicotinic and 4-(4-hydroxypiperidin-1-yl)benzoic acids. [M+H]⁺ calcd for C₃₀H₂₉N₅O₃: 508.23. Found 507.97.

### Example 641

### N-(4-((4-(4-(4-Hydroxypiperidin-1-yl)benzamido)phenyl)amino)phenyl)-4,5,6,7-tetrahydro-1H-indole-2-carboxamide (Compound 741)

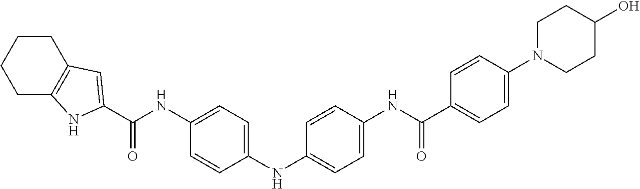 [SMILES: O=C(Nc1ccc(Nc2ccc(NC(=O)c3cc4c([nH]3)CCCC4)cc2)cc1)c1ccc(N2CCC(O)CC2)cc1]

Compound 741 was prepared according to the procedure described in Scheme IV from 4,4′-diaminodiphenylamine, 4,5,6,7-tetrahydro-1H-indole-2-carboxylic, and 4-(4-hydroxypiperidin-1-yl)benzoic acids. [M+H]⁺ calcd for C₃₃H₃₅N₅O₃: 550.27. Found 550.09.

### Example 642

### N-(4-((4-(4-(4-Hydroxypiperidin-1-yl)benzamido)phenyl)amino)phenyl)-1H-pyrrolo[2,3-b]pyridine-5-carboxamide (Compound 742)

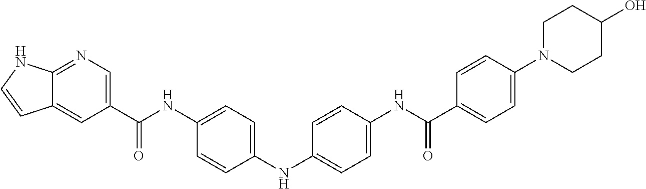 [SMILES: O=C(Nc1ccc(Nc2ccc(NC(=O)c3cnc4[nH]ccc4c3)cc2)cc1)c1ccc(N2CCC(O)CC2)cc1]

Compound 742 was prepared according to the procedure described in Scheme IV from 4,4′-diaminodiphenylamine, 1H-pyrrolo[2,3-b]pyridine-5-carboxylic, and 4-(4-hydroxypiperidin-1-yl)benzoic acids. [M+H]⁺ calcd for C₃₂H₃₀N₆O₃: 547.24. Found 547.12.

### Example 643

### N-(4-((4-(4-(4-Hydroxypiperidin-1-yl)benzamido)phenyl)amino)phenyl)-1H-pyrrole-2-carboxamide (Compound 743)

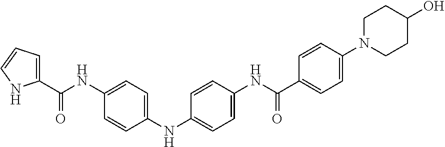 [SMILES: O=C(Nc1ccc(Nc2ccc(NC(=O)c3ccc[nH]3)cc2)cc1)c1ccc(N2CCC(O)CC2)cc1]

Compound 743 was prepared according to the procedure described in Scheme IV from 4,4′-diaminodiphenylamine, 1H-pyrrole-2-carboxylic, and 4-(4-hydroxypiperidin-1-yl)benzoic acids. [M+H]⁺ calcd for C₂₉H₂₉N₅O₃: 496.23. Found 496.02.

### Example 644

### N-(4-((4-(4-(4-Hydroxypiperidin-1-yl)benzamido)phenyl)amino)phenyl)-1-methyl-1H-pyrrole-2-carboxamide (Compound 744)

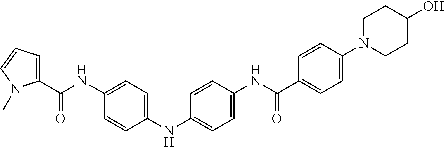 [SMILES: Cn1cccc1C(=O)Nc1ccc(Nc2ccc(NC(=O)c3ccc(N4CCC(O)CC4)cc3)cc2)cc1]

Compound 744 was prepared according to the procedure described in Scheme IV from 4,4′-diaminodiphenylamine, 1-methylpyrrole-2-carboxylic, and 4-(4-hydroxypiperidin-1-yl)benzoic acids. [M+H]⁺ calcd for C₃₀H₃₁N₅O₃: 510.24. Found 519.99.

### Example 645

### N-(4-((4-(4-(4-Hydroxypiperidin-1-yl)benzamido)phenyl)-1-methyl-N-(4-(1-methyl-1H-pyrrole-2-carboxamido)phenyl)-1H-pyrrole-2-carboxamide (Compound 745)

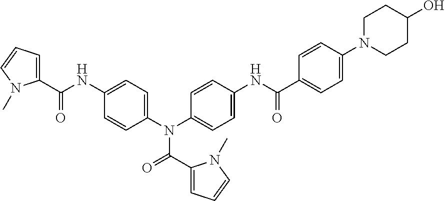 [SMILES: Cn1cccc1C(=O)Nc1ccc(N(C(=O)c2cccn2C)c2ccc(NC(=O)c3ccc(N4CCC(O)CC4)cc3)cc2)cc1]

Compound 745 was prepared according to the procedure described in Scheme IV from 4,4′-diaminodiphenylamine, 1-methylpyrrole-2-carboxylic, and 4-(4-hydroxypiperidin-1-yl)benzoic acids. [M+H]⁺ calcd for C₃₆H₃₆N₆O₄: 617.28. Found 617.27.

### Example 646

### N-(4-((4-(4-(4-Hydroxypiperidin-1-yl)benzamido)phenyl)amino)phenyl)-1H-pyrrole-3-carboxamide (Compound 746)

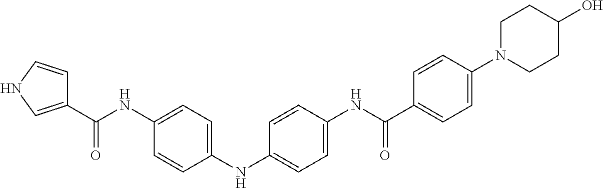 [SMILES: O=C(Nc1ccc(Nc2ccc(NC(=O)c3cc[nH]c3)cc2)cc1)c1ccc(N2CCC(O)CC2)cc1]

Compound 746 was prepared according to the procedure described in Scheme IV from 4,4′-diaminodiphenylamine, 1H-pyrrole-3-carboxylic, and 4-(4-hydroxypiperidin-1-yl)benzoic acids. [M+H]⁺ calcd for C₂₉H₂₉N₅O₃: 496.23. Found 496.02.

### Example 647

### N,N′-(Azanediylbis(4,1-phenylene))bis(4-(4H-1,2,4-triazol-4-yl)benzamide) (Compound 747)

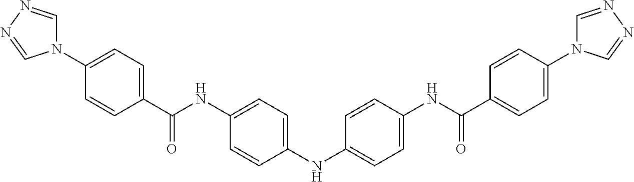 [SMILES: O=C(Nc1ccc(Nc2ccc(NC(=O)c3ccc(-n4cnnc4)cc3)cc2)cc1)c1ccc(-n2cnnc2)cc1]

Compound 747 was prepared according to the procedure described in Scheme IV from 4,4′-diaminodiphenylamine and 4-(4H-1,2,4-triazol-4-yl)benzoic acid.

[M+H]⁺ calcd for C₃₀H₂₃N₉O₂: 542.20. Found 541.99.

### Example 648

### N,N′-(Azanediylbis(4,1-phenylene))bis(4-acetylbenzamide) (Compound 748)

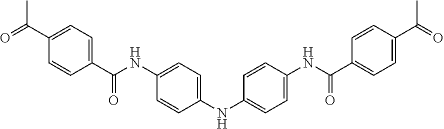 [SMILES: CC(=O)c1ccc(C(=O)Nc2ccc(Nc3ccc(NC(=O)c4ccc(C(C)=O)cc4)cc3)cc2)cc1]

Compound 748 was prepared according to the procedure described in Scheme IV from 4,4′-diaminodiphenylamine and 4-acetylbenzoic acid. [M+H]⁺ calcd for C₃₀H₂₅N₃O₄: 492.18. Found 492.00.

### Example 649

### N-(4-((4-Aminophenyl)amino)phenyl)-4-(4-hydroxypiperidin-1-yl)benzamide (Compound 749)

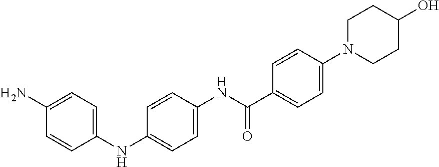 [SMILES: Nc1ccc(Nc2ccc(NC(=O)c3ccc(N4CCC(O)CC4)cc3)cc2)cc1]

Compound 749 was prepared according to the procedure described in Scheme IV from 4,4′-diaminodiphenylamine and 4-(4-hydroxypiperidin-1-yl)benzoic acid. [M+H]⁺ calcd for C₂₅H₂₆N₄O₃: 403.21. Found 403.04.

### Example 650

### N-(4-((4-(4-(4-Hydroxypiperidin-1-yl)benzamido)phenyl)amino)phenyl)-1H-indole-5-carboxamide (Compound 750)

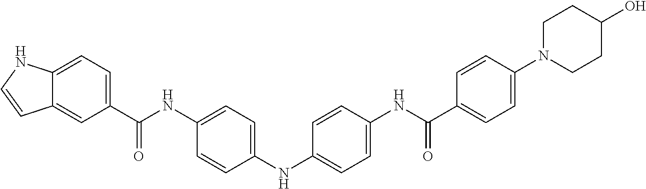 [SMILES: O=C(Nc1ccc(Nc2ccc(NC(=O)c3ccc4[nH]ccc4c3)cc2)cc1)c1ccc(N2CCC(O)CC2)cc1]

Compound 750 was prepared according to the procedure described in Scheme IV from 4,4′-diaminodiphenylamine, 1H-indole-5-carboxylic, and 4-(4-hydroxypiperidin-1-yl)benzoic acids. [M+H]⁺ calcd for C₃₃H₃₁N₅O₃: 546.24. Found 546.04.

### Example 651

### N-(4-((4-(4-(4-Hydroxypiperidin-1-yl)benzamido)phenyl)amino)phenyl)-1H-indole-6-carboxamide (Compound 751)

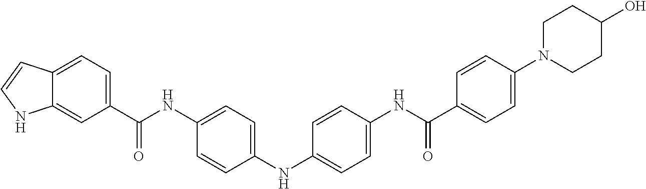 [SMILES: O=C(Nc1ccc(Nc2ccc(NC(=O)c3ccc4cc[nH]c4c3)cc2)cc1)c1ccc(N2CCC(O)CC2)cc1]

Compound 751 was prepared according to the procedure described in Scheme IV from 4,4′-diaminodiphenylamine, 1H-indole-6-carboxylic, and 4-(4-hydroxypiperidin-1-yl)benzoic acids. [M+H]⁺ calcd for C₃₃H₃₁N₅O₃: 546.24. Found 546.11.

### Example 652

### N-(4-((4-(4-(4-Hydroxypiperidin-1-yl)benzamido)phenyl)amino)phenyl)-1H-indole-3-carboxamide (Compound 752)

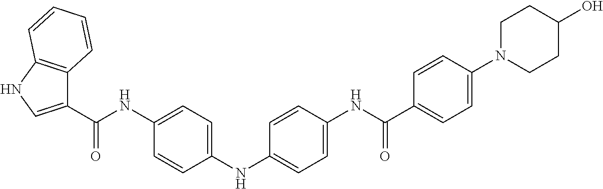 [SMILES: O=C(Nc1ccc(Nc2ccc(NC(=O)c3c[nH]c4ccccc34)cc2)cc1)c1ccc(N2CCC(O)CC2)cc1]

Compound 752 was prepared according to the procedure described in Scheme IV from 4,4′-diaminodiphenylamine, 1H-indole-3-carboxylic, and 4-(4-hydroxypiperidin-1-yl)benzoic acids. [M+H]⁺ calcd for C₃₃H₃₁N₅O₃: 546.24. Found 545.97.

### Example 653

### N-(4-((4-(4-(4-Hydroxypiperidin-1-yl)benzamido)phenyl)amino)phenyl)cyclopropanecarboxamide (Compound 753)

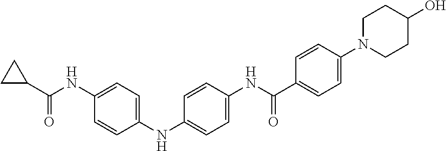 [SMILES: O=C(Nc1ccc(Nc2ccc(NC(=O)C3CC3)cc2)cc1)c1ccc(N2CCC(O)CC2)cc1]

Compound 753 was prepared according to the procedure described in Scheme IV from 4,4′-diaminodiphenylamine, cyclopropanecarboxylic, and 4-(4-hydroxypiperidin-1-yl)benzoic acids. [M+H]⁺ calcd for C₂₈H₃₀N₄O₃: 471.23. Found 471.05.

### Example 654

### N′,N′-(Azanediylbis(4,1-phenylene))diterephthalamide (Compound 754)

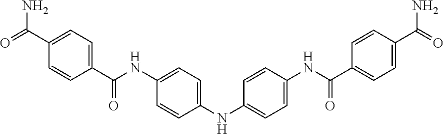 [SMILES: NC(=O)c1ccc(C(=O)Nc2ccc(Nc3ccc(NC(=O)c4ccc(C(N)=O)cc4)cc3)cc2)cc1]

Compound 754 was prepared according to the procedure described in Scheme IV from 4,4′-diaminodiphenylamine and 4-carbamoylbenzoic acid. [M+H]⁺ calcd for C₂₈H₂₃N₅O₄: 494.18. Found 494.00.

### Example 655

### N-(4-((2-Fluoro-4-(4-(4-hydroxypiperidin-1-yl)benzamido)phenyl)amino)phenyl)-1H-pyrrole-2-carboxamide (Compound 755)

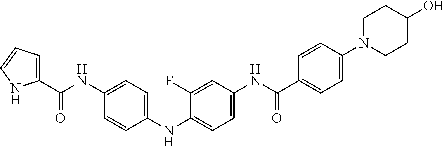 [SMILES: O=C(Nc1ccc(Nc2ccc(NC(=O)c3ccc[nH]3)cc2)c(F)c1)c1ccc(N2CCC(O)CC2)cc1]

Compound 755 was prepared according to the procedure described in Scheme IV from N¹-(4-aminophenyl)-2-fluorobenzene-1,4-diamine, 1H-pyrrole-2-carboxylic, and 4-(4-hydroxypiperidin-1-yl)benzoic acids. [M+H]⁺ calcd for C₂₉H₂₈FN₅O₃: 514.22. Found: 513.98.

### Example 656

### N-(3-Fluoro-4-((4-(4-(4-hydroxypiperidin-1-yl)benzamido)phenyl)amino)phenyl)-1H-pyrrole-2-carboxamide (Compound 756)

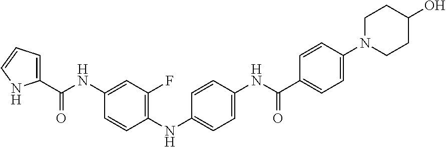 [SMILES: O=C(Nc1ccc(Nc2ccc(NC(=O)c3ccc[nH]3)cc2F)cc1)c1ccc(N2CCC(O)CC2)cc1]

Compound 756 was prepared according to the procedure described in Scheme IV from N¹-(4-aminophenyl)-2-fluorobenzene-1,4-diamine, 1H-pyrrole-2-carboxylic, and 4-(4-hydroxypiperidin-1-yl)benzoic acids. [M+H]⁺ calcd for C₂₉H₂₈FN₅O₃: 514.22. Found: 513.98.

### Example 657

### N-(4-((2-Fluoro-4-(4-(4-hydroxypiperidin-1-yl)benzamido)phenyl)amino)phenyl)-1,5-dimethyl-1H-pyrrole-2-carboxamide (Compound 757)

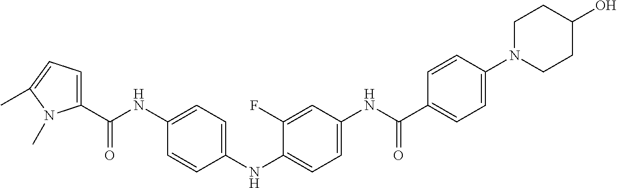 [SMILES: Cc1ccc(C(=O)Nc2ccc(Nc3ccc(NC(=O)c4ccc(N5CCC(O)CC5)cc4)cc3F)cc2)n1C]

Compound 757 was prepared according to the procedure described in Scheme IV from N¹-(4-aminophenyl)-2-fluorobenzene-1,4-diamine, 1,5-dimethyl-1H-pyrrole-2-carboxylic, and 4-(4-hydroxypiperidin-1-yl)benzoic acids. [M+H]⁺ calcd for C₃₁H₃₂FN₅O₃: 542.25. Found: 542.06.

### Example 658

### N-(4-((4-(4-(4-Hydroxypiperidin-1-yl)benzamido)phenyl)amino)phenyl)-1,5-dimethyl-1H-pyrrole-2-carboxamide (Compound 758)

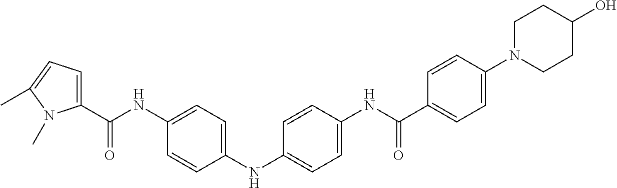 [SMILES: Cc1ccc(C(=O)Nc2ccc(Nc3ccc(NC(=O)c4ccc(N5CCC(O)CC5)cc4)cc3)cc2)n1C]

Compound 758 was prepared according to the procedure described in Scheme IV from 4,4′-diaminodiphenylamine, 1,5-dimethyl-1H-pyrrole-2-carboxylic, and 4-(4-hydroxypiperidin-1-yl)benzoic acids. [M+H]⁺ calcd for C₃₁H₃₃N₅O₃: 524.26. Found: 524.03.

### Example 659

### N-(4-((2-Fluoro-4-(4-(4-hydroxypiperidin-1-yl)benzamido)phenyl)amino)phenyl)-1-ethyl-1H-pyrrole-2-carboxamide (Compound 759)

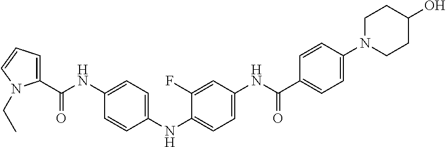 [SMILES: CCn1cccc1C(=O)Nc1ccc(Nc2ccc(NC(=O)c3ccc(N4CCC(O)CC4)cc3)cc2F)cc1]

Compound 759 was prepared according to the procedure described in Scheme IV from N¹-(4-aminophenyl)-2-fluorobenzene-1,4-diamine, 1-ethyl-1H-pyrrole-2-carboxylic, and 4-(4-hydroxypiperidin-1-yl)benzoic acids. [M+H]⁺ calcd for C₃₁H₃₂FN₅O₃: 524.25. Found: 542.06.

### Example 660

### N-(3-Fluoro-4-((4-(4-(4-hydroxypiperidin-1-yl)benzamido)phenyl)amino)phenyl)-1,5-dimethyl-1H-pyrrole-2-carboxamide (Compound 760)

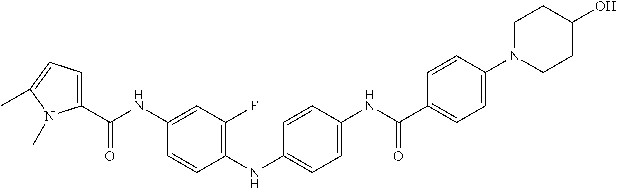 [SMILES: Cc1ccc(C(=O)Nc2ccc(Nc3ccc(NC(=O)c4ccc(N5CCC(O)CC5)cc4)cc3)c(F)c2)n1C]

Compound 760 was prepared according to the procedure described in Scheme IV from N¹-(4-aminophenyl)-2-fluorobenzene-1,4-diamine, 1,5-dimethyl-1H-pyrrole-2-carboxylic, and 4-(4-hydroxypiperidin-1-yl)benzoic acids. [M+H]⁺ calcd for C₃₁H₃₂FN₅O₃: 542.25. Found: 542.06.

### Example 661

### N-(4-((4-(4-(4-Hydroxypiperidin-1-yl)benzamido)phenyl)amino)phenyl)-5-methyl-1H-pyrrole-2-carboxamide (Compound 761)

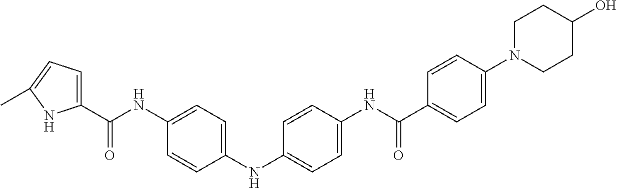 [SMILES: Cc1ccc(C(=O)Nc2ccc(Nc3ccc(NC(=O)c4ccc(N5CCC(O)CC5)cc4)cc3)cc2)[nH]1]

Compound 761 was prepared according to the procedure described in Scheme IV from 4,4′-diaminodiphenylamine, 5-methyl-1H-pyrrole-2-carboxylic, and 4-(4-hydroxypiperidin-1-yl)benzoic acids. ¹H NMR (400 MHz, Acetone-d₆) δ 10.54 (s, 1H), 9.14 (s, 1H), 8.93 (s, 1H), 7.90 (d, J=9 Hz, 2H), 7.70 (d, J=8.9 Hz, 2H), 7.64 (d, J=9.0 Hz, 2H), 7.18 (s, 1H), 7.07 (dd, J=3.0, 9.0 Hz, 4H), 7.00 (d, J=9.0 Hz, 2H), 6.84 (t, J=3.4 Hz, 1H), 5.89 (t, J=3.4 Hz, 1H), 3.85 (m, 2H), 3.75 (m, 2H), 3.08 (ddd, J=3.0, 3.2, 11 Hz, 2H), 2.32 (s, 3H), 1.94 (m, 2H), 1.60 (m, 2H).

### Example 662

### N-(3-Fluoro-4-((4-(4-(4-hydroxypiperidin-1-yl)benzamido)phenyl)amino)phenyl)-5-methyl-1H-pyrrole-2-carboxamide (Compound 762)

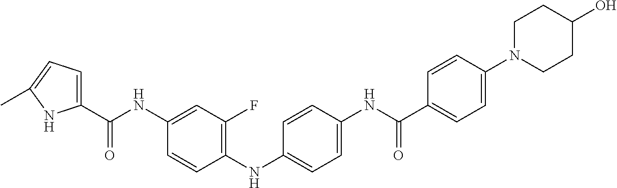 [SMILES: Cc1ccc(C(=O)Nc2ccc(Nc3ccc(NC(=O)c4ccc(N5CCC(O)CC5)cc4)cc3)c(F)c2)[nH]1]

Compound 762 was prepared according to the procedure described in Scheme IV from N¹-(4-aminophenyl)-2-fluorobenzene-1,4-diamine, 5-methyl-1H-pyrrole-2-carboxylic, and 4-(4-hydroxypiperidin-1-yl)benzoic acids. [M+H]⁺ calcd for C₃₀H₃₀FN₅O₃: 528.23. Found: 528.02.

### Example 663

### 5-Formyl-N-(4-((4-(4-(4-Hydroxypiperidin-1-yl)benzamido)phenyl)amino)phenyl)-1H-pyrrole-2-carboxamide (Compound 763)

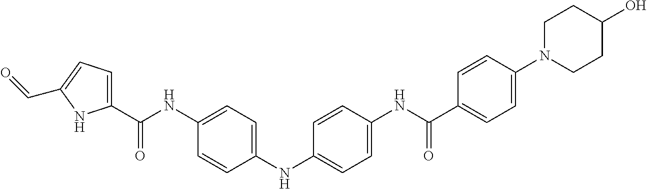 [SMILES: O=Cc1ccc(C(=O)Nc2ccc(Nc3ccc(NC(=O)c4ccc(N5CCC(O)CC5)cc4)cc3)cc2)[nH]1]

Compound 763 was prepared according to the procedure described in Scheme IV from 4,4′-diaminodiphenylamine, 5-formyl-1H-pyrrole-2-carboxylic, and 4-(4-hydroxypiperidin-1-yl)benzoic acids. [M+H]⁺ calcd for C₃₀H₂₉N₅O₄: 524.11. Found: 524.03.

### Example 664

### N-(3-Fluoro-4-((4-(4-(4-hydroxypiperidin-1-yl)benzamido)phenyl)amino)phenyl)-1H-pyrrole-3-carboxamide (Compound 764)

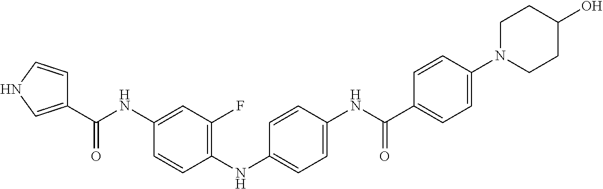 [SMILES: O=C(Nc1ccc(Nc2ccc(NC(=O)c3cc[nH]c3)cc2F)cc1)c1ccc(N2CCC(O)CC2)cc1]

Compound 764 was prepared according to the procedure described in Scheme IV from N¹-(4-aminophenyl)-2-fluorobenzene-1,4-diamine, 1H-pyrrole-3-carboxylic, and 4-(4-hydroxypiperidin-1-yl)benzoic acids. [M+H]⁺ calcd for C₂₉H₂₈FN₅O₃: 514.22. Found 513.98.

### Example 665

### N-(3-Fluoro-4-((4-(4-(4-hydroxypiperidin-1-yl)benzamido)phenyl)amino)phenyl)-1-ethyl-1H-pyrrole-3-carboxamide (Compound 765)

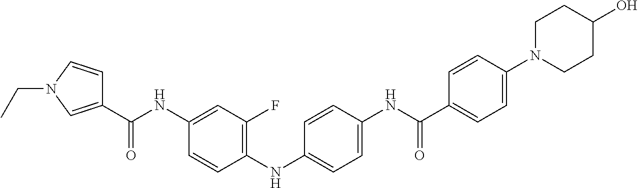 [SMILES: CCn1ccc(C(=O)Nc2ccc(Nc3ccc(NC(=O)c4ccc(N5CCC(O)CC5)cc4)cc3)c(F)c2)c1]

Compound 765 was prepared according to the procedure described in Scheme IV from N¹-(4-aminophenyl)-2-fluorobenzene-1,4-diamine, 1-ethyl-1H-pyrrole-3-carboxylic, and 4-(4-hydroxypiperidin-1-yl)benzoic acids. [M+H]⁺ calcd for C₃₁H₃₂FN₅O₃: 542.25. Found: 542.06.

### Example 666

### N-(3-Fluoro-4-((4-(4-(4-hydroxypiperidin-1-yl)benzamido)phenyl)amino)phenyl)-1-methyl-1H-pyrrole-3-carboxamide (Compound 766)

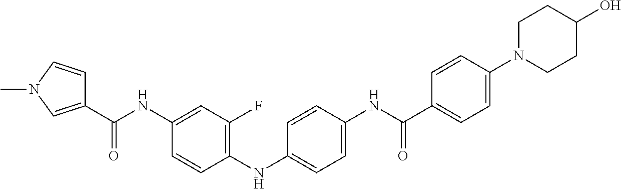 [SMILES: Cn1ccc(C(=O)Nc2ccc(Nc3ccc(NC(=O)c4ccc(N5CCC(O)CC5)cc4)cc3)c(F)c2)c1]

Compound 766 was prepared according to the procedure described in Scheme IV from N¹-(4-aminophenyl)-2-fluorobenzene-1,4-diamine, 1-methyl-1H-pyrrole-3-carboxylic, and 4-(4-hydroxypiperidin-1-yl)benzoic acids. [M+H]⁺ calcd for C₃₀H₃₀FN₅O₃: 528.23. Found 528.02.

### Example 667

### N-(4-((4-(4-(4-Hydroxypiperidin-1-yl)benzamido)phenyl)amino)phenyl)-1-ethyl-1H-pyrrole-3-carboxamide (Compound 767)

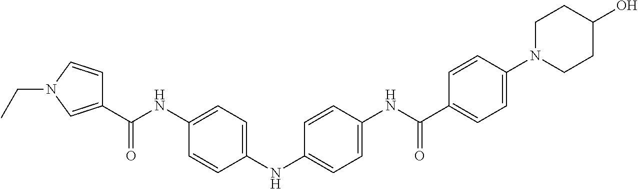 [SMILES: CCn1ccc(C(=O)Nc2ccc(Nc3ccc(NC(=O)c4ccc(N5CCC(O)CC5)cc4)cc3)cc2)c1]

Compound 767 was prepared according to the procedure described in Scheme IV from 4,4′-diaminodiphenylamine, 1-ethyl-1H-pyrrole-3-carboxylic, and 4-(4-hydroxypiperidin-1-yl)benzoic acids. [M+H]⁺ calcd for C₃₁H₃₃N₅O₃: 524.26. Found: 524.03.

### Example 668

### N-(4-((4-(4-(4-Hydroxypiperidin-1-yl)benzamido)phenyl)amino)phenyl)-1-methyl-1H-pyrrole-3-carboxamide (Compound 768)

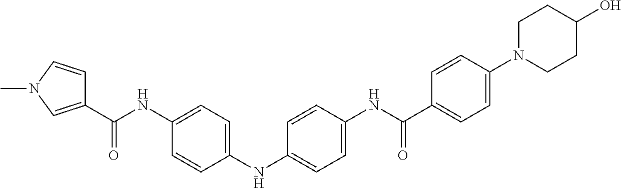 [SMILES: Cn1ccc(C(=O)Nc2ccc(Nc3ccc(NC(=O)c4ccc(N5CCC(O)CC5)cc4)cc3)cc2)c1]

Compound 768 was prepared according to the procedure described in Scheme IV from 4,4′-diaminodiphenylamine, 1-methyl-1H-pyrrole-3-carboxylic, and 4-(4-hydroxypiperidin-1-yl)benzoic acids. [M+H]⁺ calcd for C₃₀H₃₁N₅O₃: 510.24. Found 509.99.

### Example 669

### N-Cyclopropyl-4-((4-(4-(4-hydroxypiperidin-1-yl)benzamido)phenyl)amino)benzamide (Compound 769)

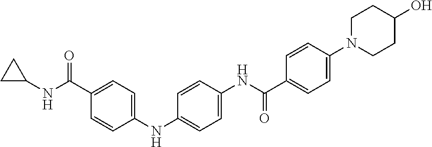 [SMILES: O=C(Nc1ccc(Nc2ccc(C(=O)NC3CC3)cc2)cc1)c1ccc(N2CCC(O)CC2)cc1]

Compound 769 was prepared according to the procedure described in Scheme IV from cyclopropylamine, 4-(4-aminophenylamino)benzoic, and 4-(4-hydroxypiperidin-1-yl)benzoic acids. [M+H]⁺ calcd for C₂₈H₃₀N₄O₃: 471.23. Found 471.05.

### Example 670

### N-(4-((4-(4-(4-Hydroxypiperidin-1-yl)benzamido)phenyl)amino)phenyl)-6-methylpicolinamide (Compound 770)

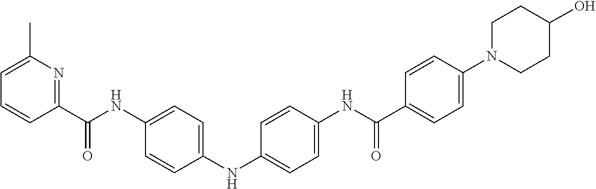 [SMILES: Cc1cccc(C(=O)Nc2ccc(Nc3ccc(NC(=O)c4ccc(N5CCC(O)CC5)cc4)cc3)cc2)n1]

Compound 770 was prepared according to the procedure described in Scheme IV from 4,4′-diaminodiphenylamine, 6-methylpicolinic, and 4-(4-hydroxypiperidin-1-yl)benzoic acids. [M+H]⁺ calcd for C₃₁H₃₁N₅O₃: 522.24. Found 522.08.

### Example 671

### N-(4-((4-((1H-Indol-6-yl)carbamoyl)phenyl)amino)phenyl)-4-(4-hydroxypiperidin-1-yl)benzamide (Compound 771)

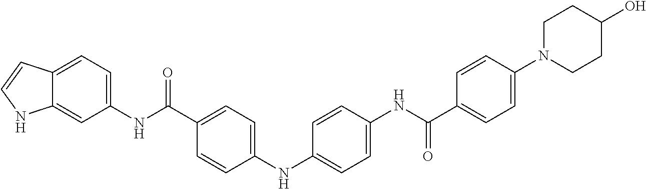 [SMILES: O=C(Nc1ccc(Nc2ccc(C(=O)Nc3ccc4cc[nH]c4c3)cc2)cc1)c1ccc(N2CCC(O)CC2)cc1]

[Compound 771 was prepared according to the procedure described in Scheme IV from 6-aminoindole, 4-(4-aminophenylamino)benzoic, and 4-(4-hydroxypiperidin-1-yl)benzoic acids. [M+H]⁺ calcd for C₃₃H₃₁N₅O₃: 546.24. Found 546.04.

### Example 672

### N-(4-((4-(4-(4-Hydroxypiperidin-1-yl)benzamido)phenyl)amino)phenyl)-1-(2-hydroxyethyl)-1H-indole-6-carboxamide (Compound 772)

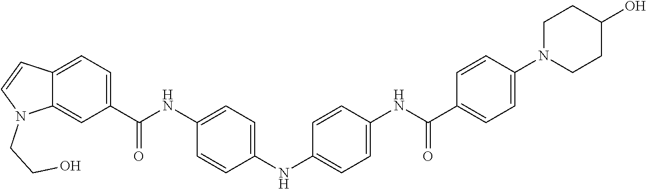 [SMILES: O=C(Nc1ccc(Nc2ccc(NC(=O)c3ccc4ccn(CCO)c4c3)cc2)cc1)c1ccc(N2CCC(O)CC2)cc1]

Compound 772 was prepared according to the procedure described in Scheme IV from 4,4′-diaminodiphenylamine, 1-(2-hydroxyethyl)-1H-indole-6-carboxylic, and 4-(4-hydroxypiperidin-1-yl)benzoic acids. [M+H]⁺ calcd for C₃₅H₃₅N₅O₄: 590.27. Found 590.20.

### Example 673

### N-(4-((4-(4-(4-Hydroxypiperidin-1-yl)benzamido)phenyl)amino)phenyl)-1-methyl-1H-indole-2-carboxamide (Compound 773)

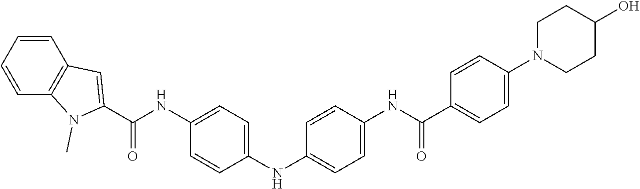 [SMILES: Cn1c(C(=O)Nc2ccc(Nc3ccc(NC(=O)c4ccc(N5CCC(O)CC5)cc4)cc3)cc2)cc2ccccc21]

Compound 773 was prepared according to the procedure described in Scheme IV from 4,4′-diaminodiphenylamine, 1-methyl-1H-indole-2-carboxylic, and 4-(4-hydroxypiperidin-1-yl)benzoic acids. [M+H]⁺ calcd for C₃₄H₃₃N₅O₃: 560.26. Found 560.02.

### Example 674

### N-(4-((4-(4-(4-Hydroxypiperidin-1-yl)benzamido)phenyl)amino)phenyl)-1-methyl-1H-indole-3-carboxamide (Compound 774)

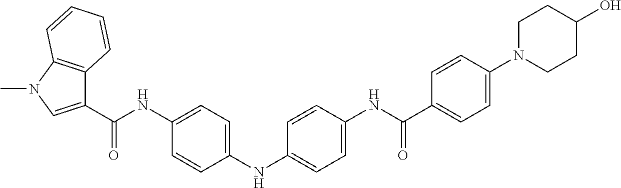 [SMILES: Cn1cc(C(=O)Nc2ccc(Nc3ccc(NC(=O)c4ccc(N5CCC(O)CC5)cc4)cc3)cc2)c2ccccc21]

Compound 774 was prepared according to the procedure described in Scheme IV from 4,4′-diaminodiphenylamine, 1-methyl-1H-indole-3-carboxylic, and 4-(4-hydroxypiperidin-1-yl)benzoic acids. [M+H]⁺ calcd for C₃₄H₃₃N₅O₃: 560.26. Found 560.02.

### Example 675

### N-(4-((4-(4-(4-Hydroxypiperidin-1-yl)benzamido)phenyl)amino)phenyl)-1-(2-hydroxyethyl)-1H-indole-5-carboxamide (Compound 775)

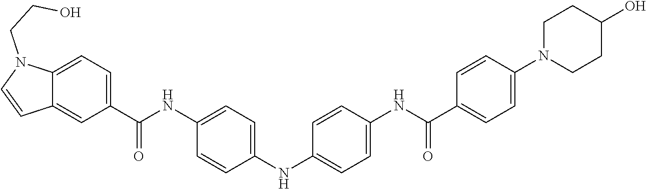 [SMILES: O=C(Nc1ccc(Nc2ccc(NC(=O)c3ccc4c(ccn4CCO)c3)cc2)cc1)c1ccc(N2CCC(O)CC2)cc1]

Compound 775 was prepared according to the procedure described in Scheme IV from 4,4′-diaminodiphenylamine, 1-(2-hydroxyethyl)-1H-indole-5-carboxylic, and 4-(4-hydroxypiperidin-1-yl)benzoic acids. [M+H]⁺ calcd for C₃₅H₃₅N₅O₄: 590.27. Found 590.06.

### Example 676

### N-(4-((4-(1H-Pyrrole-2-carboxamido)phenyl)amino)phenyl)-1-(2-hydroxyethyl)-1H-indole-6-carboxamide (Compound 776)

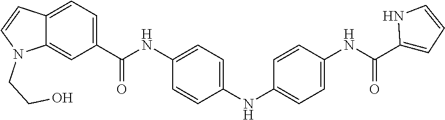 [SMILES: O=C(Nc1ccc(Nc2ccc(NC(=O)c3ccc[nH]3)cc2)cc1)c1ccc2ccn(CCO)c2c1]

Compound 776 was prepared according to the procedure described in Scheme IV from 4,4′-diaminodiphenylamine, 1-(2-hydroxyethyl)-1H-indole-6-carboxylic, and 2-pyrrolecarboxylic acids. [M+H]⁺ calcd for C₂₈H₂₅N₅O₃: 480.20. Found 480.03.

### Example 677

### N-(4-((4-(1H-Pyrrole-2-carboxamido)phenyl)amino)phenyl)-1-(2-hydroxyethyl)-1-methyl-1H-indole-6-carboxamide (Compound 777)

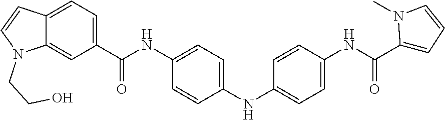 [SMILES: Cn1cccc1C(=O)Nc1ccc(Nc2ccc(NC(=O)c3ccc4ccn(CCO)c4c3)cc2)cc1]

Compound 777 was prepared according to the procedure described in Scheme IV from 4,4′-diaminodiphenylamine, 1-(2-hydroxyethyl)-1H-indole-6-carboxylic, and 1-methyl-2-pyrrolecarboxylic acids. [M+H]⁺ calcd for C₂₉H₂₇N₅O₃: 494.21. Found 494.06.

### Example 678

### (±)-N-(4-((4-(4-(3-(Hydroxymethyl)piperidin-1-yl)benzamido)phenyl)amino)phenyl)-1H-indole-6-carboxamide (Compound 778)

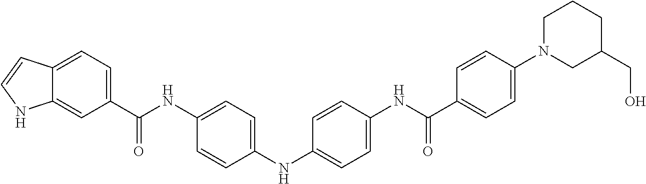 [SMILES: O=C(Nc1ccc(Nc2ccc(NC(=O)c3ccc4cc[nH]c4c3)cc2)cc1)c1ccc(N2CCCC(CO)C2)cc1]

Compound 778 was prepared according to the procedure described in Scheme IV from 4,4′-diaminodiphenylamine, 1H-indole-6-carboxylic, and 4-(3-hydroxymethylpiperidin-1-yl)benzoic acids. [M+H]⁺ calcd for C₃₄H₃₃N₅O₃: 560.26. Found: 560.08.

### Example 679

### (±)-N-(4-((4-(4-(3-(Hydroxymethyl)piperidin-1-yl)benzamido)phenyl)amino)phenyl)-1H-pyrrole-2-carboxamide (Compound 779)

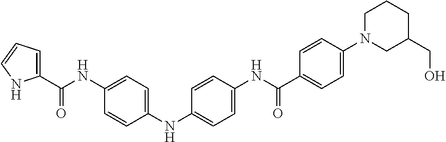 [SMILES: O=C(Nc1ccc(Nc2ccc(NC(=O)c3ccc[nH]3)cc2)cc1)c1ccc(N2CCCC(CO)C2)cc1]

Compound 779 was prepared according to the procedure described in Scheme IV from 4,4′-diaminodiphenylamine, 1H-pyrrole-2-carboxylic, and 4-(3-hydroxymethylpiperidin-1-yl)benzoic acids. [M+H]⁺ calcd for C₃₀H₃₁N₅O₃: 510.24. Found: 509.99.

### Example 680

### N-(4-((4-(4-(4-Hydroxypiperidin-1-yl)benzamido)phenyl)amino)phenyl)-1-(2-morpholinoethyl)-1H-indole-5-carboxamide (Compound 780)

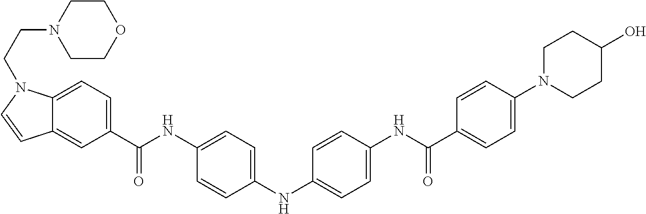 [SMILES: O=C(Nc1ccc(Nc2ccc(NC(=O)c3ccc4c(ccn4CCN4CCOCC4)c3)cc2)cc1)c1ccc(N2CCC(O)CC2)cc1]

Compound 780 was prepared according to the procedure described in Scheme IV from 4,4′-diaminodiphenylamine, 1-(2-morpholinoethyl)-1H-indole-5-carboxylic, and 4-(4-hydroxypiperidin-1-yl)benzoic acids. [M+H]⁺ calcd for C₃₉H₄₂N₆O₄: 659.33. Found 659.20.

### Example 681

### N-(4-((4-(4-(4-Hydroxypiperidin-1-yl)benzamido)phenyl)amino)phenyl)morpholine-4-carboxamide (Compound 781)

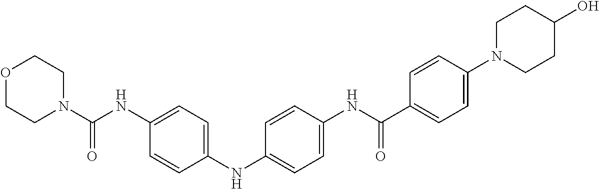 [SMILES: O=C(Nc1ccc(Nc2ccc(NC(=O)N3CCOCC3)cc2)cc1)c1ccc(N2CCC(O)CC2)cc1]

Compound 781 was prepared according to the procedure described in Scheme IV from 4,4′-diaminodiphenylamine, 4-morpholinecarbonyl chloride, and 4-(4-hydroxypiperidin-1-yl)benzoic acid. [M+H]⁺ calcd for C₂₉H₃₃N₅O₄: 516.25. Found 516.07.

### Example 682

### N-(4-((4-(4-(4-Hydroxypiperidin-1-yl)benzamido)phenyl)amino)phenyl)-1H-pyrazole-3-carboxamide (Compound 782)

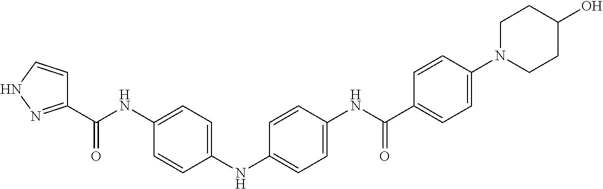 [SMILES: O=C(Nc1ccc(Nc2ccc(NC(=O)c3cc[nH]n3)cc2)cc1)c1ccc(N2CCC(O)CC2)cc1]

Compound 782 was prepared according to the procedure described in Scheme IV from 4,4′-diaminodiphenylamine, 1H-pyrazole-3-carboxylic, and 4-(4-hydroxypiperidin-1-yl)benzoic acids. [M+H]⁺ calcd for C₂₈H₂₈N₆O₃: 497.22. Found 496.97.

### Example 683

### N-(4-((4-(4-(4-Hydroxypiperidin-1-yl)benzamido)phenyl)amino)phenyl)-1H-pyrazole-4-carboxamide (Compound 783)

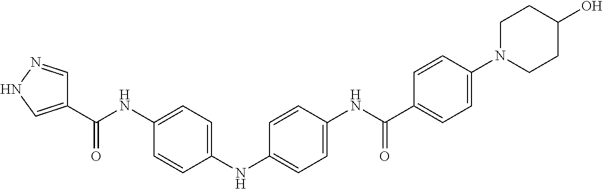 [SMILES: O=C(Nc1ccc(Nc2ccc(NC(=O)c3cn[nH]c3)cc2)cc1)c1ccc(N2CCC(O)CC2)cc1]

Compound 783 was prepared according to the procedure described in Scheme IV from 4,4′-diaminodiphenylamine, 1H-pyrazole-4-carboxylic, and 4-(4-hydroxypiperidin-1-yl)benzoic acids. [M+H]⁺ calcd for C₂₈H₂₈N₆O₃: 497.22. Found 496.97.

### Example 684

### N-(4-((4-(4-(4-Hydroxypiperidin-1-yl)benzamido)phenyl)amino)phenyl)-1-methyl-1H-pyrazole-3-carboxamide (Compound 784)

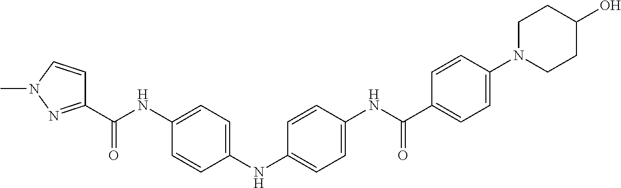 [SMILES: Cn1ccc(C(=O)Nc2ccc(Nc3ccc(NC(=O)c4ccc(N5CCC(O)CC5)cc4)cc3)cc2)n1]

Compound 784 was prepared according to the procedure described in Scheme IV from 4,4′-diaminodiphenylamine, 1-methyl-1H-pyrazole-3-carboxylic, and 4-(4-hydroxypiperidin-1-yl)benzoic acids. [M+H]⁺ calcd for C₂₉H₃₀N₆O₃: 511.24. Found 511.01.

### Example 685

### N-(4-((4-(4-(4-Hydroxypiperidin-1-yl)benzamido)phenyl)amino)phenyl)-1-methyl-1H-pyrazole-4-carboxamide (Compound 785)

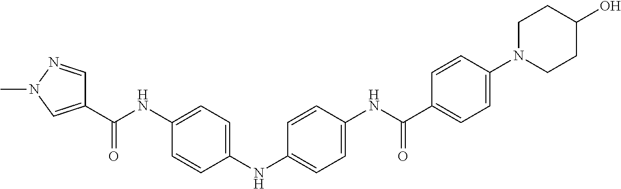 [SMILES: Cn1cc(C(=O)Nc2ccc(Nc3ccc(NC(=O)c4ccc(N5CCC(O)CC5)cc4)cc3)cc2)cn1]

Compound 785 was prepared according to the procedure described in Scheme IV from 4,4′-diaminodiphenylamine, 1-methyl-1H-pyrazole-4-carboxylic, and 4-(4-hydroxypiperidin-1-yl)benzoic acids. [M+H]⁺ calcd for C₂₉H₃₀N₆O₃: 511.24. Found 511.01.

### Example 686

### N-(4-((4-(4-(4-Hydroxypiperidin-1-yl)benzamido)phenyl)amino)phenyl)-1-methyl-1H-pyrazole-5-carboxamide (Compound 786)

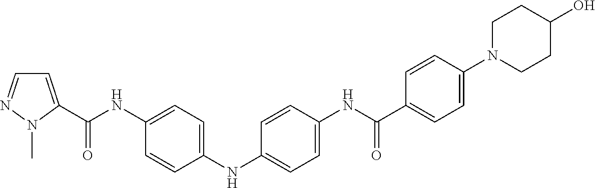 [SMILES: Cn1nccc1C(=O)Nc1ccc(Nc2ccc(NC(=O)c3ccc(N4CCC(O)CC4)cc3)cc2)cc1]

Compound 786 was prepared according to the procedure described in Scheme IV from 4,4′-diaminodiphenylamine, 1-methyl-1H-pyrazole-5-carboxylic, and 4-(4-hydroxypiperidin-1-yl)benzoic acids. [M+H]⁺ calcd for C₂₉H₃₀N₆O₃: 511.24. Found 511.01.

### Example 687

### N-(4-((4-(4-(4-Hydroxypiperidin-1-yl)benzamido)phenyl)amino)phenyl)butamide (Compound 787)

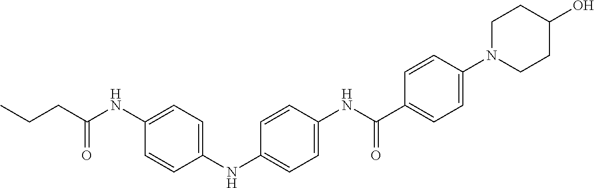 [SMILES: CCCC(=O)Nc1ccc(Nc2ccc(NC(=O)c3ccc(N4CCC(O)CC4)cc3)cc2)cc1]

Compound 787 was prepared according to the procedure described in Scheme IV from 4,4′-diaminodiphenylamine, butanoic, and 4-(4-hydroxypiperidin-1-yl)benzoic acids. [M+H]⁺ calcd for C₂₈H₃₂N₄O₃: 473.25. Found: 473.01.

### Example 688

### N-(4-((4-(4-(4-Hydroxypiperidin-1-yl)benzamido)phenyl)amino)phenyl)-4-methyl-1H-pyrrole-3-carboxamide (Compound 788)

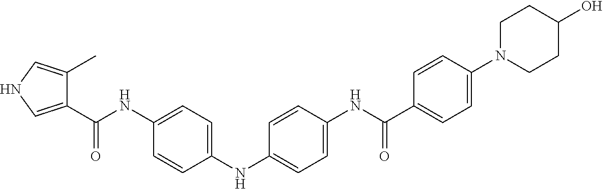 [SMILES: Cc1c[nH]cc1C(=O)Nc1ccc(Nc2ccc(NC(=O)c3ccc(N4CCC(O)CC4)cc3)cc2)cc1]

Compound 788 was prepared according to the procedure described in Scheme IV from 4,4′-diaminodiphenylamine, 4-methyl-1H-pyrrole-3-carboxylic, and 4-(4-hydroxypiperidin-1-yl)benzoic acids. [M+H]⁺ calcd for C₃₀H₃₁N₅O₃: 510.24. Found: 509.99.

### Example 689

### N-(4-((4-(4-(4-Hydroxypiperidin-1-yl)benzamido)phenyl)amino)phenyl)-1,2,5-trimethyl-1H-pyrrole-3-carboxamide (Compound 789)

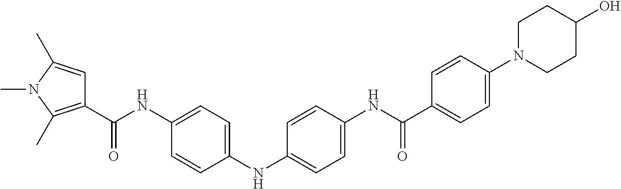 [SMILES: Cc1cc(C(=O)Nc2ccc(Nc3ccc(NC(=O)c4ccc(N5CCC(O)CC5)cc4)cc3)cc2)c(C)n1C]

Compound 789 was prepared according to the procedure described in Scheme IV from 4,4′-diaminodiphenylamine, 1,2,5-trimethyl-1H-pyrrole-3-carboxylic, and 4-(4-hydroxypiperidin-1-yl)benzoic acids. [M+H]⁺ calcd for C₃₂H₃₅N₅O₃: 538.27. Found 538.08.

### Example 690

### N-(4-((4-(4-(4-Hydroxypiperidin-1-yl)benzamido)phenyl)amino)phenyl)-2,5-dimethyl-1H-pyrrole-3-carboxamide (Compound 790)

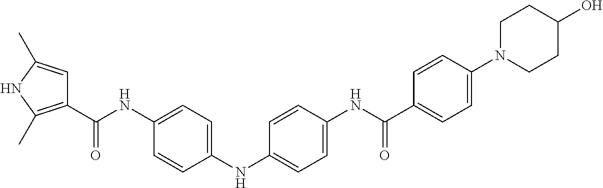 [SMILES: Cc1cc(C(=O)Nc2ccc(Nc3ccc(NC(=O)c4ccc(N5CCC(O)CC5)cc4)cc3)cc2)c(C)[nH]1]

Compound 790 was prepared according to the procedure described in Scheme IV from 4,4′-diaminodiphenylamine, 2,5-dimethyl-1H-pyrrole-3-carboxylic, and 4-(4-hydroxypiperidin-1-yl)benzoic acids. [M+H]⁺ calcd for C₃₁H₃₃N₅O₃: 524.26. Found 525.10.

### Example 691

### N-(4-((4-(3,3-Dimethylureido)phenyl)amino)phenyl)-4-(4-hydroxypiperidin-1-yl)benzamide (Compound 791)

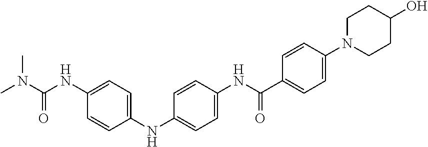 [SMILES: CN(C)C(=O)Nc1ccc(Nc2ccc(NC(=O)c3ccc(N4CCC(O)CC4)cc3)cc2)cc1]

Compound 791 was prepared according to the procedure described in Scheme IV from 4,4′-diaminodiphenylamine, N,N-dimethylchloroformate, and 4-(4-hydroxypiperidin-1-yl)benzoic acid. [M+H]⁺ calcd for C₂₇H₃₁N₅O₃: 474.24. Found 473.95.

### Example 692

### 4-Hydroxy-N-(4-((4-(4-(4-hydroxypiperidin-1-yl)benzamido)phenyl)amino)phenyl)piperidine-1-carboxamide (Compound 792)

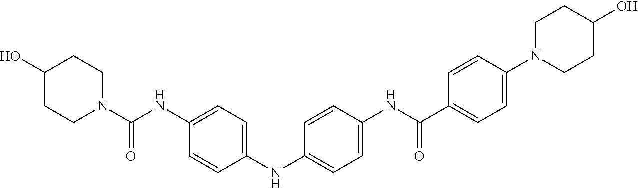 [SMILES: O=C(Nc1ccc(Nc2ccc(NC(=O)N3CCC(O)CC3)cc2)cc1)c1ccc(N2CCC(O)CC2)cc1]

Compound 792 was prepared according to the procedure described in Scheme IV from 4,4′-diaminodiphenylamine, (4-hydroxypiperidin-1-yl)carbonyl chloride, and 4-(4-hydroxypiperidin-1-yl)benzoic acid. [M+H]⁺ calcd for C₃₀H₃₅N₅O₄: 530.28. Found: 530.04.

### Example 693

### N-(4-((4-(4-(4-Hydroxypiperidin-1-yl)benzamido)phenyl)amino)phenyl)-2,3-dimethyl-1H-indole-5-carboxamide (Compound 793)

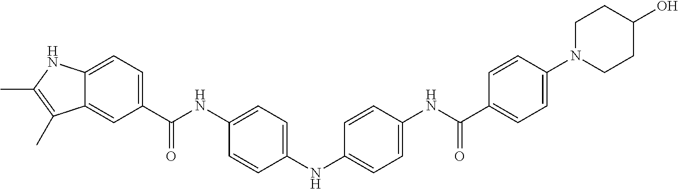 [SMILES: Cc1[nH]c2ccc(C(=O)Nc3ccc(Nc4ccc(NC(=O)c5ccc(N6CCC(O)CC6)cc5)cc4)cc3)cc2c1C]

Compound 793 was prepared according to the procedure described in Scheme IV from 4,4′-diaminodiphenylamine, 2,3-dimethyl-1H-indole-5-carboxylic, and 4-(4-hydroxypiperidin-1-yl)benzoic acids. [M+H]⁺ calcd for C₃₅H₃₅N₅O₃: 574.27. Found 574.06.

### Example 694

### N-(4-((4-(4-(4-Hydroxypiperidin-1-yl)benzamido)phenyl)amino)phenyl)-1-methyl-1H-indole-5-carboxamide (Compound 794)

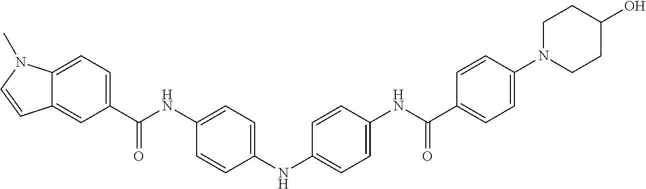 [SMILES: Cn1ccc2cc(C(=O)Nc3ccc(Nc4ccc(NC(=O)c5ccc(N6CCC(O)CC6)cc5)cc4)cc3)ccc21]

Compound 794 was prepared according to the procedure described in Scheme IV from 4,4′-diaminodiphenylamine, 1-methyl-1H-indole-5-carboxylic, and 4-(4-hydroxypiperidin-1-yl)benzoic acids. [M+H]⁺ calcd for C₃₄H₃₃N₅O₃: 560.26. Found 560.02.

### Example 695

### N-(4-((4-(4-(4-Hydroxypiperidin-1-yl)benzamido)phenyl)amino)phenyl)-1-methyl-1H-indole-6-carboxamide (Compound 795)

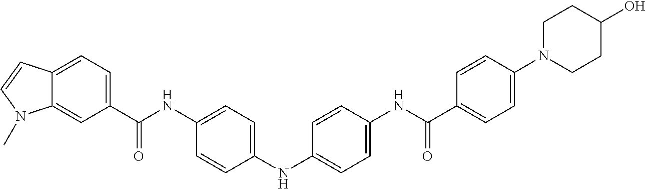 [SMILES: Cn1ccc2ccc(C(=O)Nc3ccc(Nc4ccc(NC(=O)c5ccc(N6CCC(O)CC6)cc5)cc4)cc3)cc21]

Compound 795 was prepared according to the procedure described in Scheme IV from 4,4′-diaminodiphenylamine, 1-methyl-1H-indole-6-carboxylic, and 4-(4-hydroxypiperidin-1-yl)benzoic acids. [M+H]⁺ calcd for C₃₄H₃₃N₅O₃: 560.26. Found 560.07.

### Example 696

### N-(3-Chloro-4-((4-(4-(4-hydroxypiperidin-1-yl)benzamido)phenyl)amino)phenyl)-1-methyl-1H-pyrrole-2-carboxamidee (Compound 796)

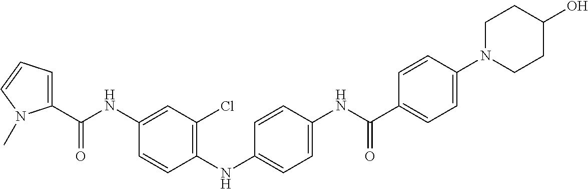 [SMILES: Cn1cccc1C(=O)Nc1ccc(Nc2ccc(NC(=O)c3ccc(N4CCC(O)CC4)cc3)cc2)c(Cl)c1]

Compound 796 was prepared according to the procedure described in Scheme IV from N¹-(4-aminophenyl)-2-chlorobenzene-1,4-diamine, 1-methyl-1H-pyrrole-2-carboxylic, and 4-(4-hydroxypiperidin-1-yl)benzoic acids. [M+H]⁺ calcd for C₃₀H₃₀ClN₅O₃: 544.20. Found 544.02.

### Example 697

### N-(4-((4-(4-((2-Hydroxyethyl)(methyl)amino)benzamido)phenyl)amino)phenyl)-1-methyl-1H-pyrrole-2-carboxamide (Compound 797)

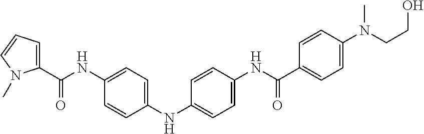 [SMILES: CN(CCO)c1ccc(C(=O)Nc2ccc(Nc3ccc(NC(=O)c4cccn4C)cc3)cc2)cc1]

Compound 797 was prepared according to the procedure described in Scheme IV from 4,4′-diaminodiphenylamine, 1-methyl-1H-pyrrole-2-carboxylic, and (4-(2-hydroxyethyl)methylamino)benzoic acids. [M+H]⁺ calcd for C₂₈H₂₉N₅O₃: 484.24. Found: 484.01.

### Example 698

### 1-Methyl-N-(4-((4-(4-(4-methylpiperazin-1-yl)benzamido)phenyl)amino)phenyl)-1H-pyrrole-2-carboxamide (Compound 798)

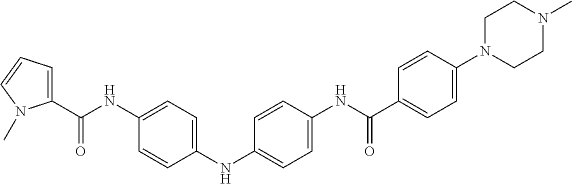 [SMILES: CN1CCN(c2ccc(C(=O)Nc3ccc(Nc4ccc(NC(=O)c5cccn5C)cc4)cc3)cc2)CC1]

Compound 798 was prepared according to the procedure described in Scheme IV from 4,4′-diaminodiphenylamine, 1-methyl-1H-pyrrole-2-carboxylic, and 4-(4-methylpiperazin-1-yl)benzoic acids. [M+H]⁺ calcd for C₃₀H₃₂N₆O₂: 509.27. Found: 509.05.

### Example 699

### N-(4-((4-(4-(4-Hydroxypiperidin-1-yl)benzamido)phenyl)amino)phenyl)-4-acetyl-3,5-dimethyl-1H-pyrrole-2-carboxamide (Compound 799)

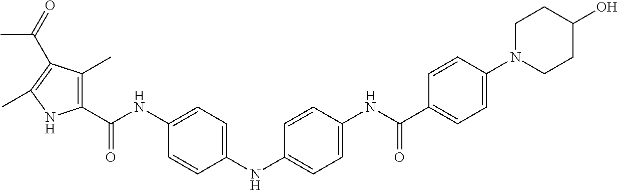 [SMILES: CC(=O)c1c(C)[nH]c(C(=O)Nc2ccc(Nc3ccc(NC(=O)c4ccc(N5CCC(O)CC5)cc4)cc3)cc2)c1C]

Compound 799 was prepared according to the procedure described in Scheme IV from 4,4′-diaminodiphenylamine, 4-acetyl-3,5-dimethyl-1H-pyrrole-2-carboxylic, and 4-(4-hydroxypiperidin-1-yl)benzoic acids. [M+H]⁺ calcd for C₃₃H₃₅N₅O₄: 566.27. Found 566.09.

### Example 700

### N-(4-((4-(4-(4-Hydroxymethylpiperidin-1-yl)benzamido)phenyl)amino)phenyl)-1-methyl-1H-pyrrole-2-carboxamide (Compound 800)

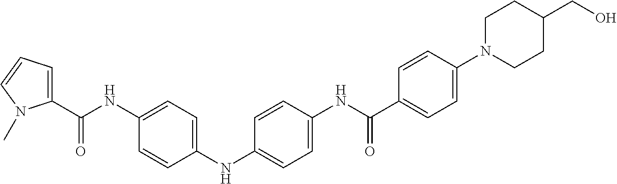 [SMILES: Cn1cccc1C(=O)Nc1ccc(Nc2ccc(NC(=O)c3ccc(N4CCC(CO)CC4)cc3)cc2)cc1]

Compound 800 was prepared according to the procedure described in Scheme IV from 4,4′-diaminodiphenylamine, 1-methyl-1H-pyrrole-2-carboxylic, and 4-(4-hydroxymethylpiperidin-1-yl)benzoic acids. [M+H]⁺ calcd for C₃₁H₃₃N₅O₃: 524.26. Found: 523.64.

### Example 701

### N-(4-((4-(4-(4-Hydroxypiperidin-1-yl)benzamido)phenyl)amino)phenyl)-1H-imidazole-2-carboxamide (Compound 801)

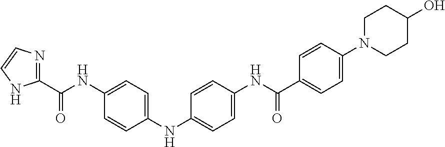 [SMILES: O=C(Nc1ccc(Nc2ccc(NC(=O)c3ncc[nH]3)cc2)cc1)c1ccc(N2CCC(O)CC2)cc1]

Compound 801 was prepared according to the procedure described in Scheme IV from 4,4′-diaminodiphenylamine, 1H-imidazole-2-carboxylic, and 4-(4-hydroxypiperidin-1-yl)benzoic acids. [M+H]⁺ calcd for C₂₈H₂₈N₆O₃: 497.22. Found 496.97.

### Example 702

### N-(4-((4-(4-(4-Hydroxypiperidin-1-yl)benzamido)phenyl)amino)phenyl)-1-methyl-1H-imidazole-4-carboxamide (Compound 802)

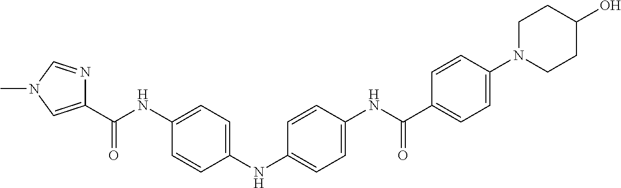 [SMILES: Cn1cnc(C(=O)Nc2ccc(Nc3ccc(NC(=O)c4ccc(N5CCC(O)CC5)cc4)cc3)cc2)c1]

Compound 802 was prepared according to the procedure described in Scheme IV from 4,4′-diaminodiphenylamine, 1-methyl-1H-imidazole-4-carboxylic, and 4-(4-hydroxypiperidin-1-yl)benzoic acids. [M+H]⁺ calcd for C₂₉H₃₀N₆O₃: 511.24. Found 511.01.

### Example 703

### N-(4-((4-(4-(4-Hydroxypiperidin-1-yl)benzamido)phenyl)amino)phenyl)-1-methyl-1H-imidazole-5-carboxamide (Compound 803)

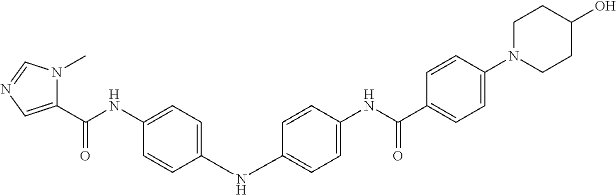 [SMILES: Cn1cncc1C(=O)Nc1ccc(Nc2ccc(NC(=O)c3ccc(N4CCC(O)CC4)cc3)cc2)cc1]

Compound 803 was prepared according to the procedure described in Scheme IV from 4,4′-diaminodiphenylamine, 1-methyl-1H-imidazole-5-carboxylic, and 4-(4-hydroxypiperidin-1-yl)benzoic acids. [M+H]⁺ calcd for C₂₉H₃₀N₆O₃: 511.24. Found 511.01.

### Example 704

### tert-Butyl 4-(4-((4-((4-(4-(4-hydroxypiperidin-1-yl)benzamido)phenyl)amino)phenyl) carbamoyl)phenyl)piperazine-1-carboxylate (Compound 804)

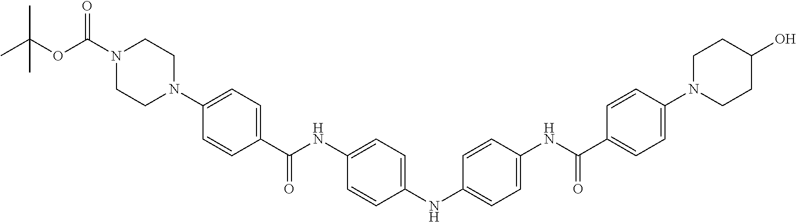 [SMILES: CC(C)(C)OC(=O)N1CCN(c2ccc(C(=O)Nc3ccc(Nc4ccc(NC(=O)c5ccc(N6CCC(O)CC6)cc5)cc4)cc3)cc2)CC1]

Compound 804 was prepared according to the procedure described in Scheme IV from 4,4′-diaminodiphenylamine, 4-(4-t-BOC-piperazin-1-yl)benzoic, and 4-(4-hydroxypiperidin-1-yl)benzoic acids. [M+H]⁺ calcd for C₄₀H₄₆N₆O₅: 691.35. Found 691.23.

### Example 705

### (±)-Benzyl 2-((4-((4-(4-(4-hydroxypiperidin-1-yl)benzamido)phenyl)amino)phenyl) carbamoyl)pyrrolidine-1-carboxylate (Compound 805)

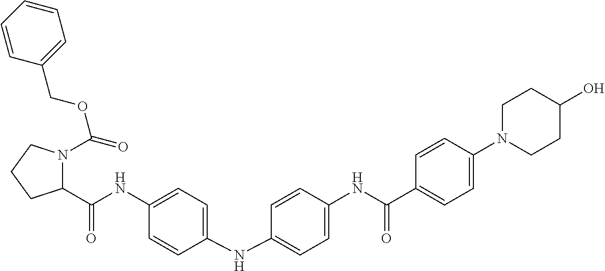 [SMILES: O=C(Nc1ccc(Nc2ccc(NC(=O)C3CCCN3C(=O)OCc3ccccc3)cc2)cc1)c1ccc(N2CCC(O)CC2)cc1]

Compound 805 was prepared according to the procedure described in Scheme IV from 4,4′-diaminodiphenylamine, 1-benzyloxycarbanylpyrrolidine-2-carboxylic, and 4-(4-hydroxypiperidin-1-yl)benzoic acids. [M+H]⁺ calcd for C₃₇H₃₉N₅O₅: 634.30. Found 634.15.

### Example 706

### N-(4-((4-(1H-Pyrrole-2-carboxamido)phenyl)amino)phenyl)-4-morpholino-1H-pyrrole-2-carboxamide (Compound 806)

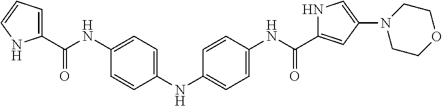 [SMILES: O=C(Nc1ccc(Nc2ccc(NC(=O)c3cc(N4CCOCC4)c[nH]3)cc2)cc1)c1ccc[nH]1]

Compound 806 was prepared according to the procedure described in Scheme IV from 4,4′-diaminodiphenylamine, 1H-pyrrole-2-carboxylic, and 4-morpholino-1H-pyrrole-2-carboxylic acids. ¹H NMR (400 MHz, Acetone-d₆) δ 10.75 (s, 1H), 10.31 (s, 1H), 9.01 (s, 1H), 8.91 (s, 1H), 7.62 (d, J=8.6 Hz, 4H), 7.04 (m, 5H), 6.67 (s, 1H), 6.58 (s, 1H), 6.18 (s, 1H), 4.04 (dq, J=1.8, 7 Hz, 2H), 3.72 (m, 4H), 1.19 (dq, J=1.8, 7 Hz, 4H).

### Example 707

### N-(4-((4-Amino-2-fluorophenyl)amino)phenyl)-4-(4-hydroxypiperidin-1-yl)benzamide (Compound 807)

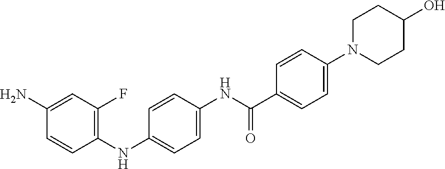 [SMILES: Nc1ccc(Nc2ccc(NC(=O)c3ccc(N4CCC(O)CC4)cc3)cc2)c(F)c1]

Compound 807 was prepared according to the procedure described in Scheme IV from N¹-(4-aminophenyl)-2-fluorobenzene-1,4-diamine and 4-(4-hydroxypiperidin-1-yl)benzoic acid. [M+H]⁺ calcd for C₂₄H₂₅FN₄O₂: 421.20. Found 420.98.

### Example 708

### N-(3-Fluoro-4-((4-(4-(4-hydroxypiperidin-1-yl)benzamido)phenyl)amino)phenyl)-1-methyl-1H-pyrrole-2-carboxamide (Compound 808)

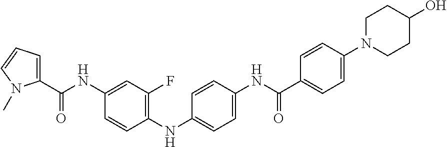 [SMILES: Cn1cccc1C(=O)Nc1ccc(Nc2ccc(NC(=O)c3ccc(N4CCC(O)CC4)cc3)cc2)c(F)c1]

Compound 808 was prepared according to the procedure described in Scheme IV from N¹-(4-aminophenyl)-2-fluorobenzene-1,4-diamine, 1-methyl-1H-pyrrole-2-carboxylic, and 4-(4-hydroxypiperidin-1-yl)benzoic acids. [M+H]⁺ calcd for C₃₀H₃₀FN₅O₃: 528.23. Found 528.02.

### Example 709

### N-(3-Fluoro-4-((4-(4-(4-hydroxypiperidin-1-yl)benzamido)phenyl)amino)phenyl)-1-methyl-1H-indole-6-carboxamide (Compound 809)

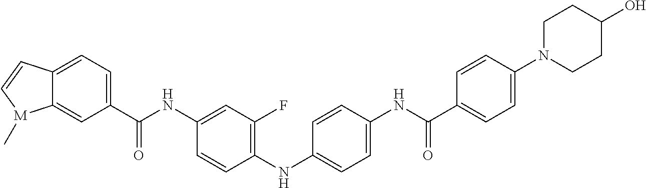

Compound 809 was prepared according to the procedure described in Scheme IV from N¹-(4-aminophenyl)-2-fluorobenzene-1,4-diamine, 1-methyl-1H-indole-6-carboxylic, and 4-(4-hydroxypiperidin-1-yl)benzoic acids. [M+H]⁺ calcd for C₃₄H₃₂FN₅O₃: 578.25. Found 578.04.

### Example 710

### (±)-N-(4-((4-(4-(3-Hydroxypyrrolidin-1-yl)benzamido)phenyl)amino)phenyl)-1-methyl-1H-pyrrole-2-carboxamide (Compound 810)

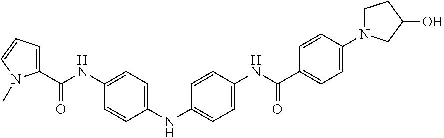 [SMILES: Cn1cccc1C(=O)Nc1ccc(Nc2ccc(NC(=O)c3ccc(N4CCC(O)C4)cc3)cc2)cc1]

Compound 810 was prepared according to the procedure described in Scheme IV from 4,4′-diaminodiphenylamine, 1-methyl-1H-pyrrole-2-carboxylic, and 4-(3-hydroxypyrrolidin-1-yl)benzoic acids. [M+H]⁺ calcd for C₂₉H₂₉N₅O₃: 496.23. Found: 495.95.

### Example 711

### (S)—N-(4-((4-(4-(2-(Hydroxymethyl)pyrrolidin-1-yl)benzamido)phenyl)amino)phenyl)-1-methyl-1H-pyrrole-2-carboxamide (Compound 811)

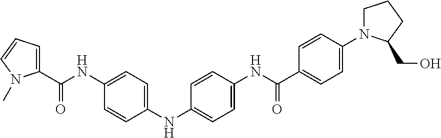 [SMILES: Cn1cccc1C(=O)Nc1ccc(Nc2ccc(NC(=O)c3ccc(N4CCC[C@H]4CO)cc3)cc2)cc1]

Compound 811 was prepared according to the procedure described in Scheme IV from 4,4′-diaminodiphenylamine, 1-methyl-1H-pyrrole-2-carboxylic, and (S)-4-(2-hydroxymethylpyrrolidin-1-yl)benzoic acids. [M+H]⁺ calcd for C₃₀H₃₁N₅O₃: 510.24. Found: 510.06.

### Example 712

### N-(4-((4-(4-(2-Hydroxyethyl)benzamido)phenyl)amino)phenyl)-1-methyl-1H-pyrrole-2-carboxamide (Compound 812)

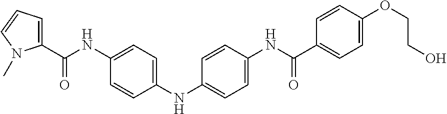 [SMILES: Cn1cccc1C(=O)Nc1ccc(Nc2ccc(NC(=O)c3ccc(OCCO)cc3)cc2)cc1]

Compound 812 was prepared according to the procedure described in Scheme IV from 4,4′-diaminodiphenylamine, 1-methyl-1H-pyrrole-2-carboxylic, and 4-(2-hydroxyethyl)benzoic acids. [M+H]⁺ calcd for C₂₇H₂₆N₄O₄: 471.20. Found: 470.92.

### Example 713

### 4-Azido-N-(4-((4-(4-(4-hydroxypiperidin-1-yl)benzamido)phenyl)amino)phenyl)benzamide (Compound 813)

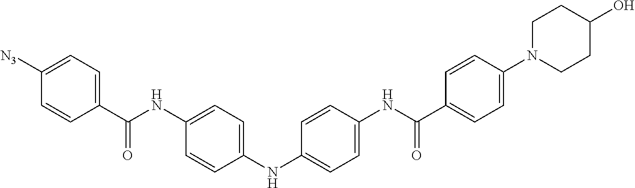 [SMILES: [N-]=[N+]=Nc1ccc(C(=O)Nc2ccc(Nc3ccc(NC(=O)c4ccc(N5CCC(O)CC5)cc4)cc3)cc2)cc1]

Compound 813 was prepared according to the procedure described in Scheme IV from 4,4′-diaminodiphenylamine, 4-azidobenzoic, and 4-(4-hydroxypiperidin-1-yl)benzoic acids. [M+H]⁺ calcd for C₃₁H₂₉N₇O₃: 548.23. Found 548.07.

### Example 714

### N-(4-((4-(4-(4-Hydroxypiperidin-1-yl)benzamido)phenyl)amino)phenyl)-1-ethyl-1H-pyrrole-2-carboxamide (Compound 814)

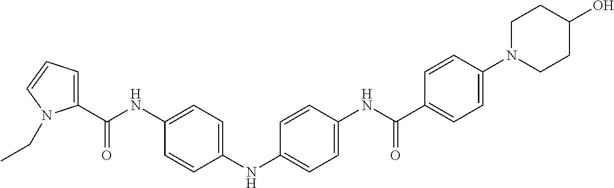 [SMILES: CCn1cccc1C(=O)Nc1ccc(Nc2ccc(NC(=O)c3ccc(N4CCC(O)CC4)cc3)cc2)cc1]

Compound 814 was prepared according to the procedure described in Scheme IV from 4,4′-diaminodiphenylamine, 1-ethyl-1H-pyrrole-2-carboxylic, and 4-(4-hydroxypiperidin-1-yl)benzoic acids. [M+H]⁺ calcd for C₃₁H₃₃N₅O₃: 524.26. Found: 524.03.

### Example 715

### N-(4-((4-(3-Fluoro-4-(4-hydroxypiperidin-1-yl)benzamido)phenyl)amino)phenyl)-1-methyl-1H-pyrrole-2-carboxamide (Compound 815)

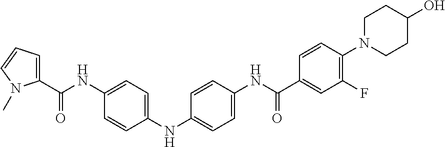 [SMILES: Cn1cccc1C(=O)Nc1ccc(Nc2ccc(NC(=O)c3ccc(N4CCC(O)CC4)c(F)c3)cc2)cc1]

Compound 815 was prepared according to the procedure described in Scheme IV from 4,4′-diaminodiphenylamine, 1-methyl-1H-pyrrole-2-carboxylic, and 3-fluoro-4-(4-hydroxypiperidin-1-yl)benzoic acids. ¹H NMR (400 MHz, Acetone-d₆) δ 9.25 (s, 1H), 8.94 (s, 1H), 7.75 (dd, J=1.9, 8.4 Hz, 1H), 7.69 (m, 3H), 7.63 (d, J=8.8 Hz, 2H), 7.22 (s, 1H), 7.08 (m, 5H), 6.89 (m, 2H), 6.05 (m, 1H), 3.95 (s, 3H), 3.47 (m, 2H), 2.95 (ddd, J=3, 9.8, 12.4 Hz, 2H), 1.67 (m, 2H), 1.27 (m, 2H).

### Example 716

### N-(4-((4-(4-(4-Hydroxypiperidin-1-yl)benzamido)-2-(trifluoromethyl)phenyl)amino)phenyl)-1-methyl-1H-pyrrole-2-carboxamide (Compound 816)

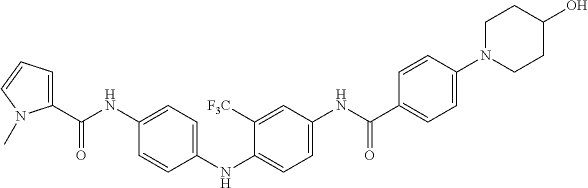 [SMILES: Cn1cccc1C(=O)Nc1ccc(Nc2ccc(NC(=O)c3ccc(N4CCC(O)CC4)cc3)cc2C(F)(F)F)cc1]

Compound 816 was prepared according to the procedure described in Scheme IV from N¹-(4-aminophenyl)-2-trifluoromethylbenzene-1,4-diamine, 1-methyl-1H-pyrrole-2-carboxylic, and 4-(4-hydroxypiperidin-1-yl)benzoic acids. [M+H]⁺ calcd for C₃₁H₃₀F₃N₅O₃: 578.24. Found: 578.04.

### Example 717

### N-(4-((4-(4-(4-Hydroxypiperidin-1-yl)benzamido)-2-(cyano)phenyl)amino)phenyl)-1-methyl-1H-pyrrole-2-carboxamide (Compound 817)

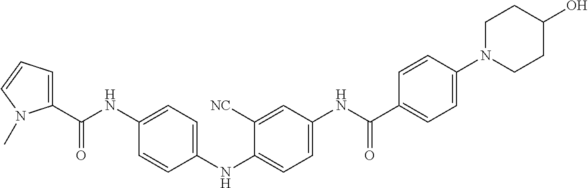 [SMILES: Cn1cccc1C(=O)Nc1ccc(Nc2ccc(NC(=O)c3ccc(N4CCC(O)CC4)cc3)cc2C#N)cc1]

Compound 817 was prepared according to the procedure described in Scheme IV from N¹-(4-aminophenyl)-2-cyanobenzene-1,4-diamine, 1-methyl-1H-pyrrole-2-carboxylic, and 4-(4-hydroxypiperidin-1-yl)benzoic acids. [M+H]⁺ calcd for C₃₁H₃₀N₆O₃: 535.25. Found: 535.04.

### Example 718

### N-(4-((4-(2-Chloro-4-(4-hydroxypiperidin-1-yl)benzamido)phenyl)amino)phenyl)-1-methyl-1H-pyrrole-2-carboxamide (Compound 818)

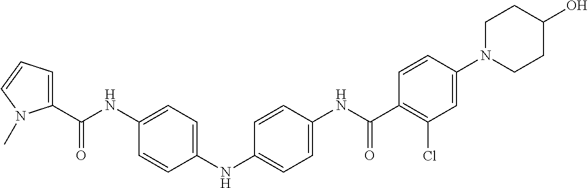 [SMILES: Cn1cccc1C(=O)Nc1ccc(Nc2ccc(NC(=O)c3ccc(N4CCC(O)CC4)cc3Cl)cc2)cc1]

Compound 818 was prepared according to the procedure described in Scheme IV from 4,4′-diaminodiphenylamine, 1-methyl-1H-pyrrole-2-carboxylic, and 2-chloro-4-(4-hydroxypiperidin-1-yl)benzoic acids. [M+H]⁺ calcd for C₃₀H₃₀ClN₅O₃: 544.20. Found: 544.02.

### Example 719

### N-(4-((4-(4-(4-Hydroxypiperidin-1-yl)benzamido)phenyl)amino)phenyl)-1-isopropyl-1H-pyrrole-2-carboxamide (Compound 819)

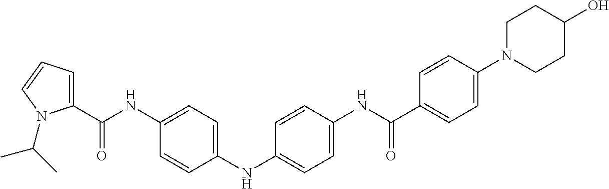 [SMILES: CC(C)n1cccc1C(=O)Nc1ccc(Nc2ccc(NC(=O)c3ccc(N4CCC(O)CC4)cc3)cc2)cc1]

Compound 819 was prepared according to the procedure described in Scheme IV from 4,4′-diaminodiphenylamine, 1-isopropyl-1H-pyrrole-2-carboxylic, and 4-(4-hydroxypiperidin-1-yl)benzoic acids. [M+H]⁺ calcd for C₃₂H₃₅N₅O₃: 538.27. Found: 538.08.

### Example 720

### N-(4-((4-(4-(4-Hydroxy-4-trifluoromethylpiperidin-1-yl)benzamido)phenyl)amino)phenyl)-1-methyl-1H-pyrrole-2-carboxamide (Compound 820)

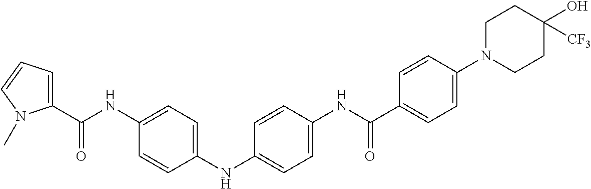 [SMILES: Cn1cccc1C(=O)Nc1ccc(Nc2ccc(NC(=O)c3ccc(N4CCC(O)(C(F)(F)F)CC4)cc3)cc2)cc1]

Compound 820 was prepared according to the procedure described in Scheme IV from 4,4′-diaminodiphenylamine, 1-methyl-1H-pyrrole-2-carboxylic, and 4-(4-hydroxy-4-trifluoromethylpiperidin-1-yl)benzoic acids. [M+H]⁺ calcd for C₃₁H₃₀F₃N₅O₃: 578.23. Found 578.04.

### Example 721

### (±)-N-(4-((4-(4-(2-Hydroxymethylmorpholi-4-no)benzamido)phenyl)amino)phenyl)-1-methyl-1H-pyrrole-2-carboxamide (Compound 821)

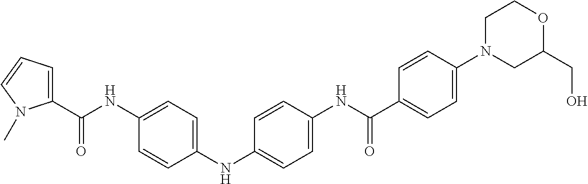 [SMILES: Cn1cccc1C(=O)Nc1ccc(Nc2ccc(NC(=O)c3ccc(N4CCOC(CO)C4)cc3)cc2)cc1]

Compound 821 was prepared according to the procedure described in Scheme IV from 4,4′-diaminodiphenylamine, 1-methyl-1H-pyrrole-2-carboxylic, and 4-(2-hydroxymethylmorpholi-4-no)benzoic acids. [M+H]⁺ calcd for C₃₀H₃₁N₅O₄: 526.24. Found: 525.99.

### Example 722

### (±)-N-(4-((4-(4-(4-Hydroxypiperidin-1-yl)benzamido)phenyl)amino)phenyl)pyrrolidine-2-carboxamide (Compound 822)

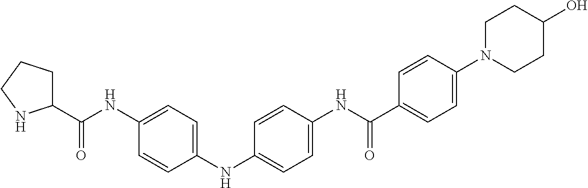 [SMILES: O=C(Nc1ccc(Nc2ccc(NC(=O)C3CCCN3)cc2)cc1)c1ccc(N2CCC(O)CC2)cc1]

Compound 822 was prepared according to the procedure described in Scheme IV from 4,4′-diaminodiphenylamine, pyrrolidine-2-carboxylic, and 4-(4-hydroxypiperidin-1-yl)benzoic acids. [M+H]⁺ calcd for C₂₉H₃₃N₅O₃: 500.26. Found 499.94.

### Example 723

### N-(4-((4-(4-(1,4-Dioxa-8-azaspiro[4.5]decan-8-yl)benzamido)phenyl)amino)phenyl)-1-methyl-1H-pyrrole-2-carboxamide (Compound 823)

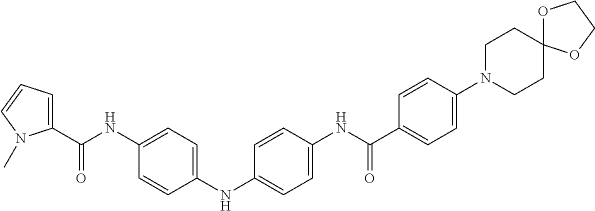 [SMILES: Cn1cccc1C(=O)Nc1ccc(Nc2ccc(NC(=O)c3ccc(N4CCC5(CC4)OCCO5)cc3)cc2)cc1]

Compound 823 was prepared according to the procedure described in Scheme IV from 4,4′-diaminodiphenylamine, 1-methyl-1H-pyrrole-2-carboxylic, and 4-(1,4-dioxa-8-azaspiro[4.5]decan-8-yl)benzoic acids. [M+H]⁺ calcd for C₃₂H₃₃N₅O₄: 552.25. Found: 552.05.

### Example 724

### N-(4-((4-(4-(4-Hydroxypiperidin-1-yl)benzamido)phenyl)amino)phenyl)-1-vinyl-1H-pyrrole-2-carboxamide (Compound 824)

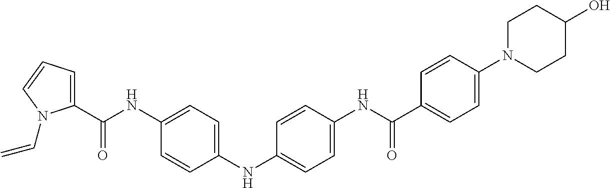 [SMILES: C=Cn1cccc1C(=O)Nc1ccc(Nc2ccc(NC(=O)c3ccc(N4CCC(O)CC4)cc3)cc2)cc1]

Compound 824 was prepared according to the procedure described in Scheme IV from 4,4′-diaminodiphenylamine, 1-vinyl-1H-pyrrole-2-carboxylic, and 4-(4-hydroxypiperidin-1-yl)benzoic acids. [M+H]⁺ calcd for C₃₁H₃₁N₅O₃: 522.24. Found 522.01.

### Example 725

### 3-Fluoro-4-(4-hydroxypiperidin-1-yl)-N-(4-((4-(4-(4-hydroxypiperidin-1-yl)benzamido) phenyl)amino)phenyl)benzamide (Compound 825)

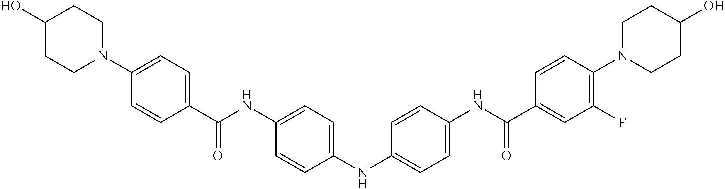 [SMILES: O=C(Nc1ccc(Nc2ccc(NC(=O)c3ccc(N4CCC(O)CC4)c(F)c3)cc2)cc1)c1ccc(N2CCC(O)CC2)cc1]

Compound 825 was prepared according to the procedure described in Scheme IV from 4,4′-diaminodiphenylamine, 3-fluoro-4-(4-hydroxypiperidin-1-yl)benzoic, and 4-(4-hydroxypiperidin-1-yl)benzoic acids. [M+H]⁺ calcd for C₃₆H₃₈FN₅O₄: 624.29. Found 624.13.

### Example 726

### N-(3-Chloro-4-((4-(4-(4-hydroxypiperidin-1-yl)benzamido)phenyl)amino)phenyl)-4-(4-hydroxypiperidin-1-yl)benzamide (Compound 826)

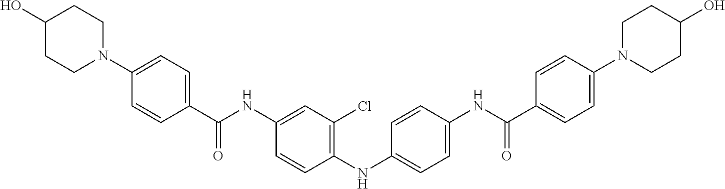 [SMILES: O=C(Nc1ccc(Nc2ccc(NC(=O)c3ccc(N4CCC(O)CC4)cc3)cc2Cl)cc1)c1ccc(N2CCC(O)CC2)cc1]

Compound 826 was prepared according to the procedure described in Scheme IV from N¹-(4-aminophenyl)-2-chlorobenzene-1,4-diamine and 4-(4-hydroxypiperidin-1-yl)benzoic acid. [M+H]⁺ calcd for C₃₆H₃₈ClN₅O₄: 640.26. Found 640.12.

### Example 727

### N-(4-((4-(4-(4-Hydroxypiperidin-1-yl)benzamido)-2-(methoxycarbonyl)phenyl)amino)phenyl)-1-methyl-1H-pyrrole-2-carboxamide (Compound 827)

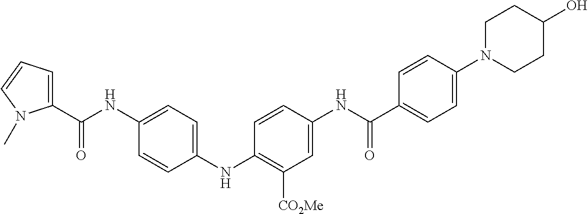 [SMILES: COC(=O)c1cc(NC(=O)c2ccc(N3CCC(O)CC3)cc2)ccc1Nc1ccc(NC(=O)c2cccn2C)cc1]

Compound 827 was prepared according to the procedure described in Scheme IV from N¹-(4-aminophenyl)-2-methoxycarbonylbenzene-1,4-diamine, 1-methyl-1H-pyrrole-2-carboxylic, and 4-(4-hydroxypiperidin-1-yl)benzoic acids. [M+H]⁺ calcd for C₃₂H₃₃N₅O₅: 568.26. Found: 568.05.

### Example 728

### 5-(4-(4-Hydroxypiperidin-1-yl)benzamido)-2-((4-(1-methyl-1H-pyrrole-2-carboxamido)phenyl)amino)benzoic acid (Compound 828)

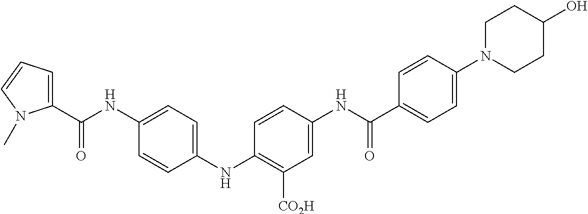 [SMILES: Cn1cccc1C(=O)Nc1ccc(Nc2ccc(NC(=O)c3ccc(N4CCC(O)CC4)cc3)cc2C(=O)O)cc1]

Compound 828 was prepared by hydrolysis of Compound 827. [M+H]⁺ calcd for C₃₁H₃₁N₅O₅: 554.24. Found: 554.01.

### Example 729

### N-(4-((4-(4-(4-Hydroxypiperidin-1-yl)benzamido)phenyl)amino)phenyl)-1-cyanomethyl-1H-pyrrole-2-carboxamide (Compound 829)

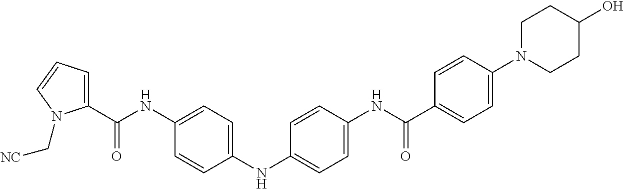 [SMILES: N#CCn1cccc1C(=O)Nc1ccc(Nc2ccc(NC(=O)c3ccc(N4CCC(O)CC4)cc3)cc2)cc1]

Compound 829 was prepared according to the procedure described in Scheme IV from 4,4′-diaminodiphenylamine, 1-cyanomethyl-1H-pyrrole-2-carboxylic, and 4-(4-hydroxypiperidin-1-yl)benzoic acids. [M+H]⁺ calcd for C₃₁H₃₀N₆O₃: 535.24. Found: 535.04.

### Example 730

### Ethyl 2-(2-chloro-5-((4-((4-(4-(4-hydroxypiperidin-1-yl)benzamido)phenyl)amino)phenyl)carbamoyl)-1H-pyrrol-1-yl)acetate (Compound 830)

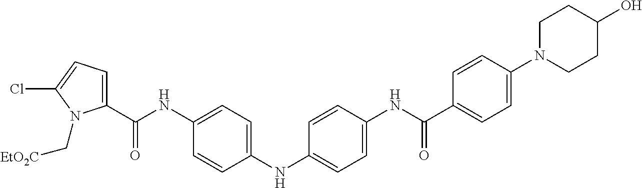 [SMILES: CCOC(=O)Cn1c(Cl)ccc1C(=O)Nc1ccc(Nc2ccc(NC(=O)c3ccc(N4CCC(O)CC4)cc3)cc2)cc1]

Compound 830 was prepared according to the procedure described in Scheme IV from 4,4′-diaminodiphenylamine, 5-chloro-1-ethoxycarbonylmethyl-H-pyrrole-2-carboxylic, and 4-(4-hydroxypiperidin-1-yl)benzoic acids. [M+H]⁺ calcd for C₃₃H₃₄ClN₅O₅: 616.22. Found: 616.06.

### Example 731

### N-(4-((4-(4-(4-Hydroxypiperidin-1-yl)benzamido)-2-fluorophenyl)amino)phenyl)-1-methyl-1H-pyrrole-2-carboxamide (Compound 831)

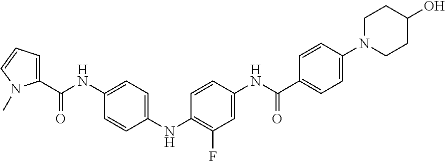 [SMILES: Cn1cccc1C(=O)Nc1ccc(Nc2ccc(NC(=O)c3ccc(N4CCC(O)CC4)cc3)cc2F)cc1]

Compound 831 was prepared according to the procedure described in Scheme IV from N¹-(4-aminophenyl)-2-fluorobenzene-1,4-diamine, 1-methyl-1H-pyrrole-2-carboxylic, and 4-(4-hydroxypiperidin-1-yl)benzoic acids. [M+H]⁺ calcd for C₃₀H₃₀FN₅O₃: 528.23. Found 528.02.

### Example 732

### N-(4-((4-(4-(4-Hydroxypiperidin-1-yl)benzamido)-2-chlorophenyl)amino)phenyl)-1-methyl-1H-pyrrole-2-carboxamide (Compound 832)

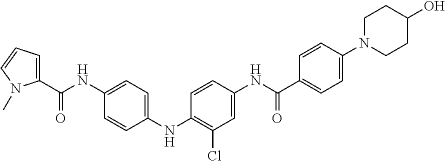 [SMILES: Cn1cccc1C(=O)Nc1ccc(Nc2ccc(NC(=O)c3ccc(N4CCC(O)CC4)cc3)cc2Cl)cc1]

Compound 832 was prepared according to the procedure described in Scheme IV from N¹-(4-aminophenyl)-2-chlorobenzene-1,4-diamine, 1-methyl-1H-pyrrole-2-carboxylic, and 4-(4-hydroxypiperidin-1-yl)benzoic acids. [M+H]⁺ calcd for C₃₀H₃₀ClN₅O₃: 544.20. Found 544.02.

### Example 733

### N-(5-((4-(4-(4-Hydroxypiperidin-1-yl)benzamido)phenyl)amino)pyridin-2-yl)-1-methyl-1H-pyrrole-2-carboxamide (Compound 833)

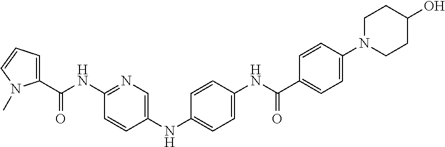 [SMILES: Cn1cccc1C(=O)Nc1ccc(Nc2ccc(NC(=O)c3ccc(N4CCC(O)CC4)cc3)cc2)cn1]

Compound 833 was prepared according to the procedure described in Scheme IV from N⁵-(4-aminophenyl)pyridine-2,5-diamine, 1-methyl-1H-pyrrole-2-carboxylic, and 4-(4-hydroxypiperidin-1-yl)benzoic acids. [M+H]⁺ calcd for C₂₉H₃₀N₆O₃: 511.24. Found 511.01.

### Example 734

### N-(3-Cyano-4-((4-(4-(4-hydroxypiperidin-1-yl)benzamido)phenyl)amino)phenyl)-4-(4-hydroxypiperidin-1-yl)benzamide (Compound 834)

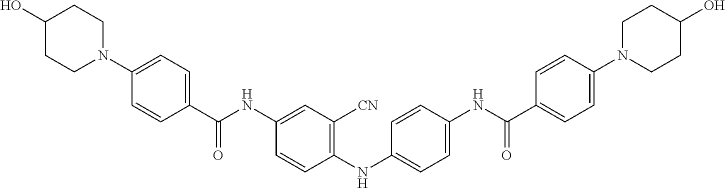 [SMILES: N#Cc1cc(NC(=O)c2ccc(N3CCC(O)CC3)cc2)ccc1Nc1ccc(NC(=O)c2ccc(N3CCC(O)CC3)cc2)cc1]

Compound 834 was prepared according to the procedure described in Scheme IV from N¹-(4-aminophenyl)-2-cyanobenzene-1,4-diamine and 4-(4-hydroxypiperidin-1-yl)benzoic acid. [M+H]⁺ calcd for C₃₇H₃₈N₆O₄: 631.30. Found: 631.11.

### Example 735

### N-(4-((4-(4-(4-(2-Hydroxyethoxy)piperidin-1-yl)benzamido)phenyl)amino)phenyl)-1-methyl-1H-pyrrole-2-carboxamide (Compound 835)

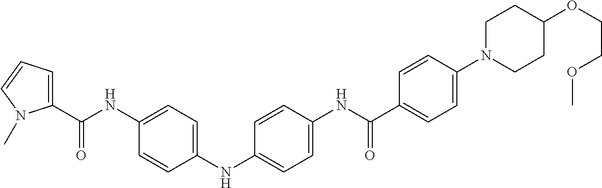 [SMILES: COCCOC1CCN(c2ccc(C(=O)Nc3ccc(Nc4ccc(NC(=O)c5cccn5C)cc4)cc3)cc2)CC1]

Compound 835 was prepared according to the procedure described in Scheme IV from 4,4′-diaminodiphenylamine, 1-methyl-1H-pyrrole-2-carboxylic, and 4-(4-(2-hydroxyethoxy)piperidin-1-yl)benzoic acids. [M+H]⁺ calcd for C₃₃H₃₇N₅O₄: 568.28. Found: 568.12.

### Example 736

### 2-(2-((4-((4-(4-(4-Hydroxypiperidin-1-yl)benzamido)phenyl)amino)phenyl)carbamoyl)-1H-pyrrol-1-yl)acetic acid (Compound 836)

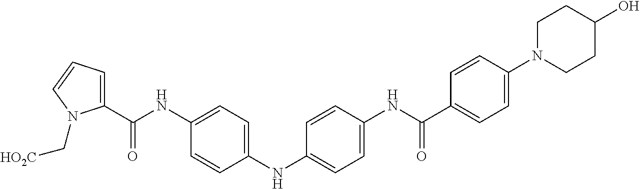 [SMILES: O=C(O)Cn1cccc1C(=O)Nc1ccc(Nc2ccc(NC(=O)c3ccc(N4CCC(O)CC4)cc3)cc2)cc1]

Compound 836 was prepared according to the procedure described in Scheme IV from 4,4′-diaminodiphenylamine, 1-methoxycarbonylmethyl-1H-pyrrole-2-carboxylic, and 4-(4-hydroxypiperidin-1-yl)benzoic acids. [M+H]⁺ calcd for C₃₁H₃₁N₅O₅: 554.23. Found: 554.01.

### Example 737

### N-(4-((4-(3-Methyl-4-(4-hydroxypiperidin-1-yl)benzamido)phenyl)amino)phenyl)-1-methyl-1H-pyrrole-2-carboxamide (Compound 837)

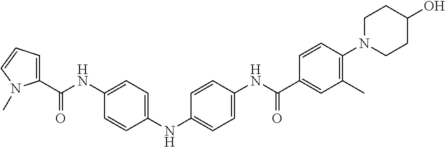 [SMILES: Cc1cc(C(=O)Nc2ccc(Nc3ccc(NC(=O)c4cccn4C)cc3)cc2)ccc1N1CCC(O)CC1]

Compound 837 was prepared according to the procedure described in Scheme IV from 4,4′-diaminodiphenylamine, 1-methyl-1H-pyrrole-2-carboxylic, and 3-methyl-4-(4-hydroxypiperidin-1-yl)benzoic acids. [M+H]⁺ calcd for C₃₁H₃₃N₅O₃: 524.26. Found: 524.03.

### Example 738

### (±)-N-(4-((4-(4-(4-(2,6-Dimethyl)morpholi-4-no)benzamido)phenyl)amino)phenyl)-1-methyl-1H-pyrrole-2-carboxamide (Compound 838)

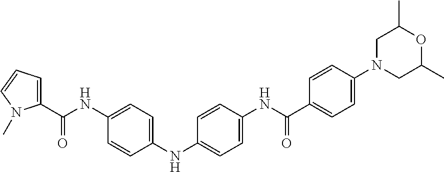 [SMILES: CC1CN(c2ccc(C(=O)Nc3ccc(Nc4ccc(NC(=O)c5cccn5C)cc4)cc3)cc2)CC(C)O1]

Compound 838 was prepared according to the procedure described in Scheme IV from 4,4′-diaminodiphenylamine, 1-methyl-1H-pyrrole-2-carboxylic, and 4-(2,6-dimethylmorpholi-4-no)benzoic acids. [M+H]⁺ calcd for C₃₁H₃₃N₅O₃: 524.26. Found: 524.03.

### Example 739

### N-(4-((4-(3-Methoxy-4-(4-hydroxypiperidin-1-yl)benzamido)phenyl)amino)phenyl)-1-methyl-1H-pyrrole-2-carboxamide (Compound 839)

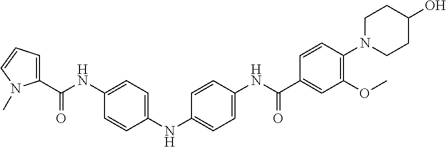 [SMILES: COc1cc(C(=O)Nc2ccc(Nc3ccc(NC(=O)c4cccn4C)cc3)cc2)ccc1N1CCC(O)CC1]

Compound 839 was prepared according to the procedure described in Scheme IV from 4,4′-diaminodiphenylamine, 1-methyl-1H-pyrrole-2-carboxylic, and 3-methoxy-4-(4-hydroxypiperidin-1-yl)benzoic acids. [M+H]⁺ calcd for C₃₁H₃₃N₅O₄: 540.25. Found: 540.03.

### Example 740

### N-(4-((4-(3-Trifluoromethyl-4-(4-hydroxypiperidin-1-yl)benzamido)phenyl)amino)phenyl)-1-methyl-1H-pyrrole-2-carboxamide (Compound 840)

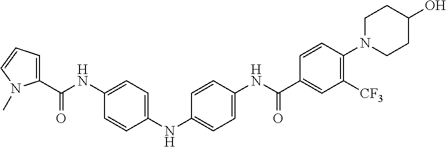 [SMILES: Cn1cccc1C(=O)Nc1ccc(Nc2ccc(NC(=O)c3ccc(N4CCC(O)CC4)c(C(F)(F)F)c3)cc2)cc1]

Compound 840 was prepared according to the procedure described in Scheme IV from 4,4′-diaminodiphenylamine, 1-methyl-1H-pyrrole-2-carboxylic, and 3-trifluoromethyl-4-(4-hydroxypiperidin-1-yl)benzoic acids. [M+H]⁺ calcd for C₃₁H₃₀F₃N₅O₃: 578.23. Found: 578.04.

### Example 741

### N-(4-((4-(4-(4-Hydroxypiperidin-1-yl)benzamido)phenyl)amino)phenyl)-2,4-dimethyl-1H-pyrrole-3-carboxamide (Compound 841)

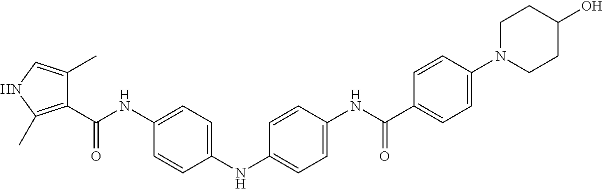 [SMILES: Cc1c[nH]c(C)c1C(=O)Nc1ccc(Nc2ccc(NC(=O)c3ccc(N4CCC(O)CC4)cc3)cc2)cc1]

Compound 841 was prepared according to the procedure described in Scheme IV from 4,4′-diaminodiphenylamine, 2,4-dimethyl-1H-pyrrole-3-carboxylic, and 4-(4-hydroxypiperidin-1-yl)benzoic acids. [M+H]⁺ calcd for C₃₁H₃₃N₅O₃: 524.26. Found 524.03.

### Example 742

### N-(4-((4-(4-(4-Hydroxypiperidin-1-yl)benzamido)phenyl)amino)phenyl)-1-(2-hydroxyethyl)-1H-pyrrole-2-carboxamide (Compound 842)

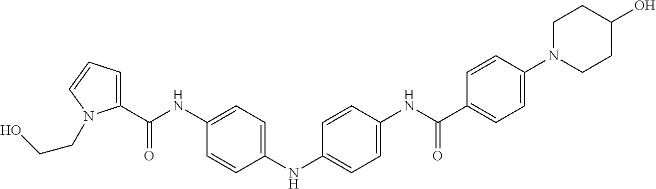 [SMILES: O=C(Nc1ccc(Nc2ccc(NC(=O)c3cccn3CCO)cc2)cc1)c1ccc(N2CCC(O)CC2)cc1]

Compound 842 was prepared according to the procedure described in Scheme IV from 4,4′-diaminodiphenylamine, 1-(2-hydroxyethyl)-1H-pyrrole-2-carboxylic, and 4-(4-hydroxypiperidin-1-yl)benzoic acids. [M+H]⁺ calcd for C₃₁H₃₃N₅O₄: 540.25. Found: 540.03.

### Example 743

### 1-Ethyl-N-(3-fluoro-4-((4-(4-(4-hydroxypiperidin-1-yl)benzamido)phenyl)amino)phenyl)-1H-pyrrole-2-carboxamide (Compound 843)

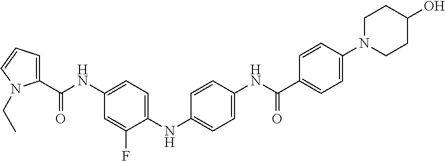 [SMILES: CCn1cccc1C(=O)Nc1ccc(Nc2ccc(NC(=O)c3ccc(N4CCC(O)CC4)cc3)cc2)c(F)c1]

Compound 843 was prepared according to the procedure described in Scheme IV from N¹-(4-aminophenyl)-2-fluorobenzene-1,4-diamine, 1-ethyl-1H-pyrrole-2-carboxylic, and 4-(4-hydroxypiperidin-1-yl)benzoic acids. [M+H]⁺ calcd for C₃₁H₃₂FN₅O₃: 542.25. Found: 542.06.

### Example 744

### N-(3-Fluoro-4-((4-(4-(4-hydroxypiperidin-1-yl)benzamido)phenyl)amino)phenyl)-1-methyl-1H-indole-2-carboxamide (Compound 844)

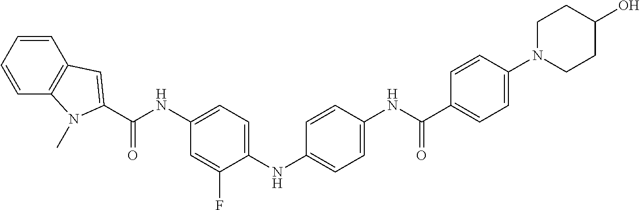 [SMILES: Cn1c(C(=O)Nc2ccc(Nc3ccc(NC(=O)c4ccc(N5CCC(O)CC5)cc4)cc3)c(F)c2)cc2ccccc21]

Compound 844 was prepared according to the procedure described in Scheme IV from N¹-(4-aminophenyl)-2-fluorobenzene-1,4-diamine, 1-methyl-1H-indole-2-carboxylic, and 4-(4-hydroxypiperidin-1-yl)benzoic acids. [M+H]⁺ calcd for C₃₄H₃₂FN₅O₃: 578.25. Found 578.11.

### Example 745

### N-(3-Fluoro-4-((4-(4-(4-hydroxypiperidin-1-yl)benzamido)phenyl)amino)phenyl)-5-methyl-1H-indole-2-carboxamide (Compound 845)

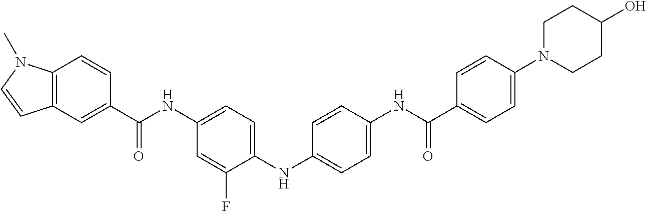 [SMILES: Cn1ccc2cc(C(=O)Nc3ccc(Nc4ccc(NC(=O)c5ccc(N6CCC(O)CC6)cc5)cc4)c(F)c3)ccc21]

Compound 845 was prepared according to the procedure described in Scheme IV from N¹-(4-aminophenyl)-2-fluorobenzene-1,4-diamine, 1-methyl-1H-indole-5-carboxylic, and 4-(4-hydroxypiperidin-1-yl)benzoic acids. [M+H]⁺ calcd for C₃₄H₃₂FN₅O₃: 578.25. Found 578.11.

### Example 746

### N-(3-Chloro-4-((4-(4-(4-hydroxypiperidin-1-yl)benzamido)phenyl)amino)phenyl)-1-methyl-1H-indole-2-carboxamide (Compound 846)

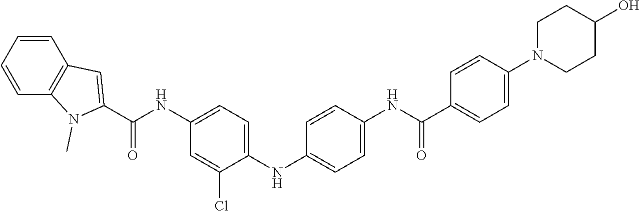 [SMILES: Cn1c(C(=O)Nc2ccc(Nc3ccc(NC(=O)c4ccc(N5CCC(O)CC5)cc4)cc3)c(Cl)c2)cc2ccccc21]

Compound 846 was prepared according to the procedure described in Scheme IV from N¹-(4-aminophenyl)-2-chlorobenzene-1,4-diamine, 1-methyl-1H-indole-2-carboxylic, and 4-(4-hydroxypiperidin-1-yl)benzoic acids. [M+H]⁺ calcd for C₃₄H₃₂ClN₅O₃: 594.22. Found 594.11.

### Example 747

### N-(3-Chloro-4-((4-(4-(4-hydroxypiperidin-1-yl)benzamido)phenyl)amino)phenyl)-5-methyl-1H-indole-2-carboxamide (Compound 847)

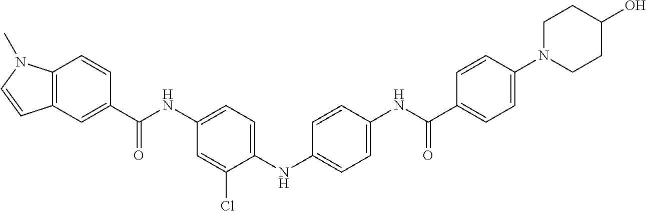 [SMILES: Cn1ccc2cc(C(=O)Nc3ccc(Nc4ccc(NC(=O)c5ccc(N6CCC(O)CC6)cc5)cc4)c(Cl)c3)ccc21]

Compound 847 was prepared according to the procedure described in Scheme IV from N¹-(4-aminophenyl)-2-chlorobenzene-1,4-diamine, 1-methyl-1H-indole-5-carboxylic, and 4-(4-hydroxypiperidin-1-yl)benzoic acids. [M+H]⁺ calcd for C₃₄H₃₂ClN₅O₃: 594.22. Found 594.11.

### Example 748

### N-(4-((2-Cyano-4-(4-(4-hydroxypiperidin-1-yl)benzamido)phenyl)amino)phenyl)-1-ethyl-1H-pyrrole-2-carboxamide (Compound 848)

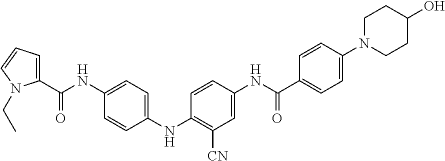 [SMILES: CCn1cccc1C(=O)Nc1ccc(Nc2ccc(NC(=O)c3ccc(N4CCC(O)CC4)cc3)cc2C#N)cc1]

Compound 848 was prepared according to the procedure described in Scheme IV from N¹-(4-aminophenyl)-2-cyanobenzene-1,4-diamine, 1-ethyl-1H-pyrrole-2-carboxylic, and 4-(4-hydroxypiperidin-1-yl)benzoic acids. [M+H]⁺ calcd for C₃₂H₃₂N₆O₃: 549.25. Found: 549.08.

### Example 749

### N-(3-Methyl-4-((4-(4-(4-hydroxypiperidin-1-yl)benzamido)phenyl)amino)phenyl)-1-methyl-1H-pyrrole-2-carboxamide (Compound 849)

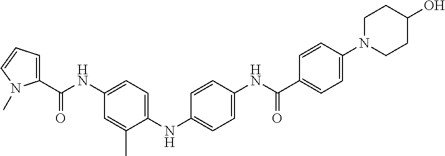 [SMILES: Cc1cc(NC(=O)c2cccn2C)ccc1Nc1ccc(NC(=O)c2ccc(N3CCC(O)CC3)cc2)cc1]

Compound 849 was prepared according to the procedure described in Scheme IV from N¹-(4-aminophenyl)-2-methylbenzene-1,4-diamine, 1-methyl-1H-pyrrole-2-carboxylic, and 4-(4-hydroxypiperidin-1-yl)benzoic acids. [M+H]⁺ calcd for C₃₁H₃₃N₅O₃: 524.26. Found 524.03.

### Example 750

### N-(4-((4-(4-(4-Hydroxypiperidin-1-yl)benzamido)phenyl)amino)-2-methylphenyl)-1-methyl-1H-pyrrole-2-carboxamide (Compound 850)

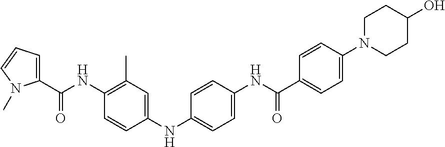 [SMILES: Cc1cc(Nc2ccc(NC(=O)c3ccc(N4CCC(O)CC4)cc3)cc2)ccc1NC(=O)c1cccn1C]

Compound 850 was prepared according to the procedure described in Scheme IV from N¹-(4-aminophenyl)-3-methylbenzene-1,4-diamine, 1-methyl-1H-pyrrole-2-carboxylic, and 4-(4-hydroxypiperidin-1-yl)benzoic acids. [M+H]⁺ calcd for C₃₁H₃₃N₅O₃: 524.26. Found 524.03.

### Example 751

### N-(4-((2-Methyl-4-(4-(4-hydroxypiperidin-1-yl)benzamido)phenyl)amino)phenyl)-1-ethyl-1H-pyrrole-2-carboxamide (Compound 851)

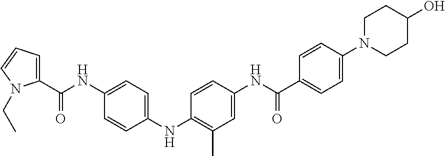 [SMILES: CCn1cccc1C(=O)Nc1ccc(Nc2ccc(NC(=O)c3ccc(N4CCC(O)CC4)cc3)cc2C)cc1]

Compound 851 was prepared according to the procedure described in Scheme IV from N¹-(4-aminophenyl)-2-methylbenzene-1,4-diamine, 1-ethyl-1H-pyrrole-2-carboxylic, and 4-(4-hydroxypiperidin-1-yl)benzoic acids. ¹H NMR (400 MHz, Acetone-d₆) δ 9.09 (s, 1H), 8.95 (s, 1H), 7.89 (d, J=9.0 Hz, 2H), 7.66 (m, 3H), 7.54 (dd. J=2.4, 8.6 Hz, 1H), 7.16 (d, J=8.6 Hz, 1H), 6.99 (m, 3H), 6.94 (dd, J=2.6, 4.0 Hz, 1H), 6.89 (d, J=9.0 Hz, 2H), 6.57 (s, 1H), 6.09 (dd, J=2.6, 4.0 Hz, 1H), 4.46 (q, J=7.1 Hz, 2H), 3.85 (m, 1H), 3.75 (dt, J=4.4, 13 Hz, 2H), 3.08 (ddd, J=3.2, 9.8, 13.0 Hz, 2H), 2.26 (s, 3H), 1.93 (m, 2H), 1.60 (m, 2H), 1.39 (t, J=7.1 Hz, 3H).

### Example 752

### N-(3-Cyano-4-((4-(4-(4-hydroxypiperidin-1-yl)benzamido)phenyl)amino)phenyl)-1-methyl-1H-pyrrole-3-carboxamide (Compound 852)

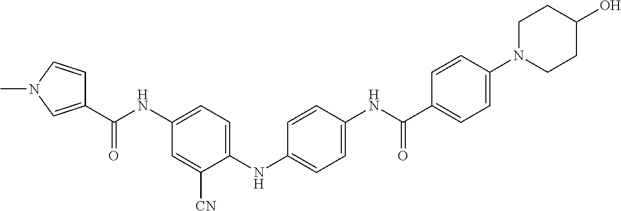 [SMILES: Cn1ccc(C(=O)Nc2ccc(Nc3ccc(NC(=O)c4ccc(N5CCC(O)CC5)cc4)cc3)c(C#N)c2)c1]

Compound 852 was prepared according to the procedure described in Scheme IV from N¹-(4-aminophenyl)-2-cyanobenzene-1,4-diamine, 1-methyl-1H-pyrrole-3-carboxylic, and 4-(4-hydroxypiperidin-1-yl)benzoic acids. [M+H]⁺ calcd for C₃₁H₃₀N₆O₃: 535.25. Found: 535.04.

### Example 753

### N-(4-((2-Cyano-4-(4-(4-hydroxypiperidin-1-yl)benzamido)phenyl)amino)phenyl)-1-methyl-1H-pyrrole-3-carboxamide (Compound 853)

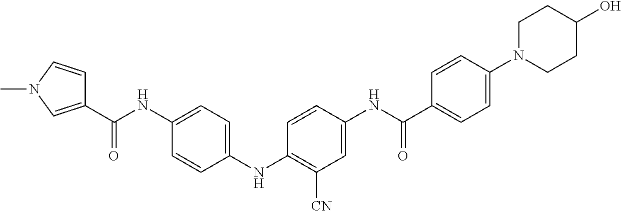 [SMILES: Cn1ccc(C(=O)Nc2ccc(Nc3ccc(NC(=O)c4ccc(N5CCC(O)CC5)cc4)cc3C#N)cc2)c1]

Compound 853 was prepared according to the procedure described in Scheme IV from N¹-(4-aminophenyl)-2-cyanobenzene-1,4-diamine, 1-methyl-1H-pyrrole-3-carboxylic, and 4-(4-hydroxypiperidin-1-yl)benzoic acids. [M+H]⁺ calcd for C₃₁H₃₀N₆O₃: 535.25. Found: 535.04.

### Example 754

### (R)—N-(4-((4-(4-(3-Hydroxypyrrolidin-1-yl)benzamido)phenyl)amino)phenyl)-1-methyl-1H-pyrrole-3-carboxamide (Compound 854)

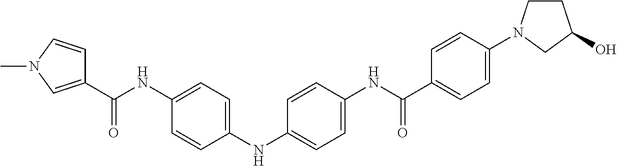 [SMILES: Cn1ccc(C(=O)Nc2ccc(Nc3ccc(NC(=O)c4ccc(N5CC[C@@H](O)C5)cc4)cc3)cc2)c1]

Compound 854 was prepared according to the procedure described in Scheme IV from 4,4′-diaminodiphenylamine, 1-methyl-1H-pyrrole-3-carboxylic, and (R)-4-(3-hydroxypyrrolidin-1-yl)benzoic acids. [M+H]⁺ calcd for C₂₉H₂₉N₅O₃: 496.23. Found: 495.95.

### Example 755

### N-(4-((4-(4-(4-Hydroxypiperidin-1-yl)benzamido)phenyl)amino)phenyl)-4,5-dimethyl-1H-pyrrole-3-carboxamide (Compound 855)

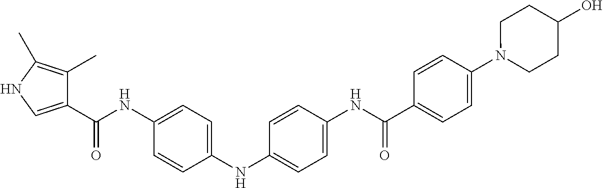 [SMILES: Cc1[nH]cc(C(=O)Nc2ccc(Nc3ccc(NC(=O)c4ccc(N5CCC(O)CC5)cc4)cc3)cc2)c1C]

Compound 855 was prepared according to the procedure described in Scheme IV from 4,4′-diaminodiphenylamine, 4,5-dimethyl-1H-pyrrole-3-carboxylic, and 4-(4-hydroxypiperidin-1-yl)benzoic acids. [M+H]⁺ calcd for C₃₁H₃₃N₅O₃: 524.26. Found: 524.03.

### Example 756

### Methyl 5-((4-((4-(4-(4-hydroxypiperidin-1-yl)benzamido)phenyl)amino)phenyl)carbamoyl)-1H-pyrrole-2-carboxylate (Compound 856)

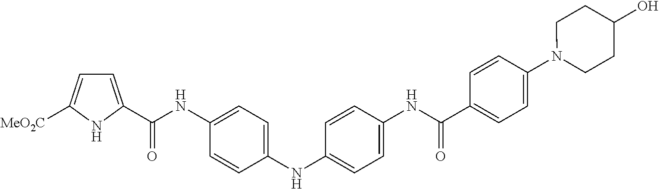 [SMILES: COC(=O)c1ccc(C(=O)Nc2ccc(Nc3ccc(NC(=O)c4ccc(N5CCC(O)CC5)cc4)cc3)cc2)[nH]1]

Compound 856 was prepared according to the procedure described in Scheme IV from 4,4′-diaminodiphenylamine, 5-methoxycarbonyl-1H-pyrrole-2-carboxylic, and 4-(4-hydroxypiperidin-1-yl)benzoic acids. [M+H]⁺ calcd for C₃₁H₃₁N₅O₅: 554.13. Found: 554.07.

### Example 757

### (R)—N-(4-((2-Fluoro-4-(4-(3-hydroxypyrrolidin-1-yl)benzamido)phenyl)amino)phenyl)-1-methyl-1H-pyrrole-3-carboxamide (Compound 857)

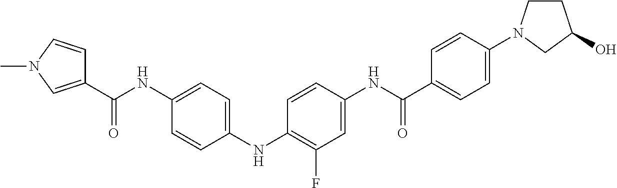 [SMILES: Cn1ccc(C(=O)Nc2ccc(Nc3ccc(NC(=O)c4ccc(N5CC[C@@H](O)C5)cc4)cc3F)cc2)c1]

Compound 857 was prepared according to the procedure described in Scheme IV from N¹-(4-aminophenyl)-2-fluorobenzene-1,4-diamine, 1-methyl-1H-pyrrole-3-carboxylic, and (R)-4-(3-hydroxypyrrolidin-1-yl)benzoic acids. [M+H]⁺ calcd for C₂₉H₂₈FN₅O₃: 514.22. Found: 513.98.

### Example 758

### N-(4-((4-(4-(4-Hydroxypiperidin-1-yl)benzamido)phenyl)amino)phenyl)-4-(methoxymethyl)-1-methyl-1H-pyrrole-2-carboxamide (Compound 858)

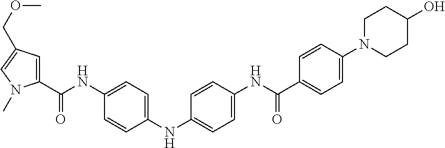 [SMILES: COCc1cc(C(=O)Nc2ccc(Nc3ccc(NC(=O)c4ccc(N5CCC(O)CC5)cc4)cc3)cc2)n(C)c1]

Compound 858 was prepared according to the procedure described in Scheme IV from 4,4′-diaminodiphenylamine, 4-methoxymethyl-1-methyl-1H-pyrrole-2-carboxylic, and 4-(4-hydroxypiperidin-1-yl)benzoic acids. [M+H]⁺ calcd for C₃₂H₃₆N₅O₄: 554.28. Found: 554.07.

### Example 759

### N-(4-((2-Fluoro-4-(4-(4-hydroxypiperidin-1-yl)benzamido)phenyl)amino)phenyl)-1-methyl-1H-pyrrole-3-carboxamide (Compound 859)

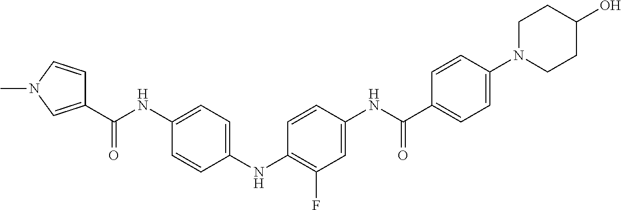 [SMILES: Cn1ccc(C(=O)Nc2ccc(Nc3ccc(NC(=O)c4ccc(N5CCC(O)CC5)cc4)cc3F)cc2)c1]

Compound 859 was prepared according to the procedure described in Scheme IV from N¹-(4-aminophenyl)-2-fluorobenzene-1,4-diamine, 1-methyl-1H-pyrrole-3-carboxylic, and 4-(4-hydroxypiperidin-1-yl)benzoic acids. [M+H]⁺ calcd for C₃₀H₃₀FN₅O₃: 528.23. Found 528.02.

### Example 760

### N-(4-((2-Fluoro-4-(4-(4-hydroxypiperidin-1-yl)benzamido)phenyl)amino)phenyl)-1H-pyrrole-3-carboxamide (Compound 860)

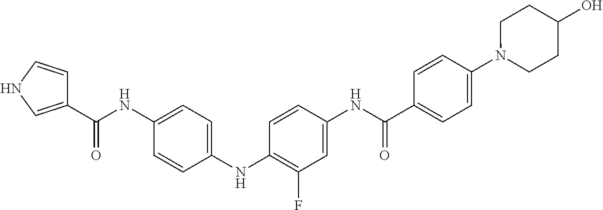 [SMILES: O=C(Nc1ccc(Nc2ccc(NC(=O)c3cc[nH]c3)cc2)c(F)c1)c1ccc(N2CCC(O)CC2)cc1]

Compound 860 was prepared according to the procedure described in Scheme IV from N¹-(4-aminophenyl)-2-fluorobenzene-1,4-diamine, 1H-pyrrole-3-carboxylic, and 4-(4-hydroxypiperidin-1-yl)benzoic acids. [M+H]⁺ calcd for C₂₉H₂₈FN₅O₃: 514.22. Found 513.98.

### Example 761

### N-(4-((4-(1H-Pyrrole-3-carboxamido)phenyl)amino)-3-fluorophenyl)-1-methyl-1H-pyrrole-2-carboxamide (Compound 861)

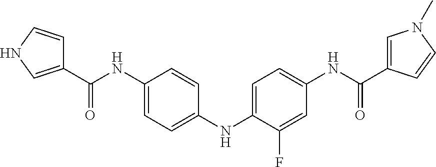 [SMILES: Cn1ccc(C(=O)Nc2ccc(Nc3ccc(NC(=O)c4cc[nH]c4)cc3)c(F)c2)c1]

Compound 861 was prepared according to the procedure described in Scheme IV from N¹-(4-aminophenyl)-2-fluorobenzene-1,4-diamine and 1H-pyrrole-3-carboxylic acid. [M+H]⁺ calcd for C₂₃H₂₀FN₅O₂: 418.16. Found 417.88.

### Example 762

### N-(4-((4-(1H-pyrrole-3-carboxamido)phenyl)amino)-3-fluorophenyl)-1H-indole-5-carboxamide (Compound 862)

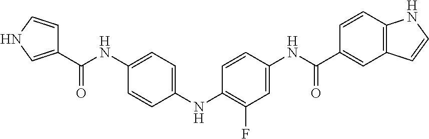 [SMILES: O=C(Nc1ccc(Nc2ccc(NC(=O)c3ccc4[nH]ccc4c3)cc2F)cc1)c1cc[nH]c1]

Compound 862 was prepared according to the procedure described in Scheme IV from N¹-(4-aminophenyl)-2-fluorobenzene-1,4-diamine, 1H-indole-5-carboxylic, and 1H-pyrrole-3-carboxylic acids. [M+H]⁺ calcd for C₂₆H₂₀FN₅O₂: 454.16. Found 453.91.

### Example 763

### N-(4-((4-(4-(Dimethylamino)benzamido)phenyl)amino)phenyl)-1H-pyrrole-3-carboxamide (Compound 863)

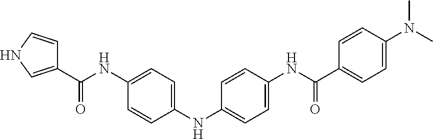 [SMILES: CN(C)c1ccc(C(=O)Nc2ccc(Nc3ccc(NC(=O)c4cc[nH]c4)cc3)cc2)cc1]

Compound 863 was prepared according to the procedure described in Scheme IV from 4,4′-diaminodiphenylamine, 4-dimethylaminobenzoic, and 1H-pyrrole-3-carboxylic acids. [M+H]⁺ calcd for C₂₆H₂₅N₅O₂: 440.20. Found 439.94.

### Example 764

### 4-Azido-N-(4-((4-(4-(dimethylamino)benzamido)phenyl)amino)phenyl)benzamide (Compound 864)

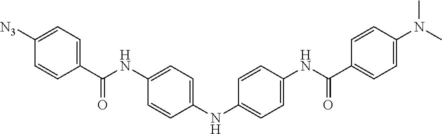 [SMILES: CN(C)c1ccc(C(=O)Nc2ccc(Nc3ccc(NC(=O)c4ccc(N=[N+]=[N-])cc4)cc3)cc2)cc1]

Compound 864 was prepared according to the procedure described in Scheme IV from 4,4′-diaminodiphenylamine, 4-dimethylaminobenzoic, and 4-azidobenzoic acids.

[M+H]⁺ calcd for C₂₈H₂₅N₇O₂: 492.21. Found 491.97.

### Example 765

### N-(4-((4-(4-(1H-Pyrrole-3-carboxamido)benzamido)phenyl)amino)phenyl)-1H-pyrrole-3-carboxamide (Compound 865)

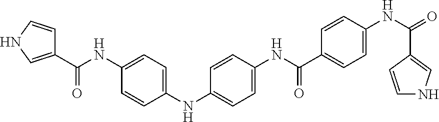 [SMILES: O=C(Nc1ccc(Nc2ccc(NC(=O)c3cc[nH]c3)cc2)cc1)c1ccc(NC(=O)c2cc[nH]c2)cc1]

Compound 865 was prepared according to the procedure described in Scheme IV from 4,4′-diaminodiphenylamine, 4-aminobenzoic, and 1H-pyrrole-3-carboxylic acids.

[M+H]⁺ calcd for C₂₉H₂₄N₆O₃: 505.19. Found 504.93.

### Example 766

### (R)—N-(4-((4-(4-(3-Hydroxypyrrolidin-1-yl)benzamido)phenyl)amino)phenyl)-2,4-dimethyl-1H-pyrrole-3-carboxamide (Compound 866)

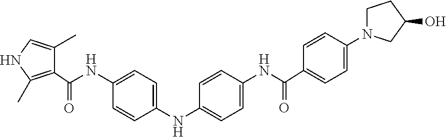 [SMILES: Cc1c[nH]c(C)c1C(=O)Nc1ccc(Nc2ccc(NC(=O)c3ccc(N4CC[C@@H](O)C4)cc3)cc2)cc1]

Compound 866 was prepared according to the procedure described in Scheme IV from 4,4′-diaminodiphenylamine, 2,4-dimethyl-1H-pyrrole-3-carboxylic, and (R)-4-(3-hydroxypyrrolidin-1-yl)benzoic acids. [M+H]⁺ calcd for C₃₀H₃₁N₅O₃: 510.24. Found: 509.99.

### Example 767

### N-(4-((4-(4-(4-Hydroxypiperidin-1-yl)benzamido)phenyl)amino)phenyl)-5-methyl-1H-pyrrole-3-carboxamide (Compound 867)

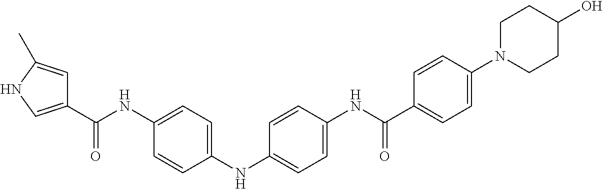 [SMILES: Cc1cc(C(=O)Nc2ccc(Nc3ccc(NC(=O)c4ccc(N5CCC(O)CC5)cc4)cc3)cc2)c[nH]1]

Compound 867 was prepared according to the procedure described in Scheme IV from 4,4′-diaminodiphenylamine, 5-methyl-1H-pyrrole-3-carboxylic, and 4-(4-hydroxypiperidin-1-yl)benzoic acids. [M+H]⁺ calcd for C₃₀H₃₁N₅O₃: 510.24. Found: 509.99.

### Example 768

### N-(3-Cyano-4-((4-(4-(4-hydroxypiperidin-1-yl)benzamido)phenyl)amino)phenyl)-1-ethyl-1H-pyrrole-2-carboxamide (Compound 868)

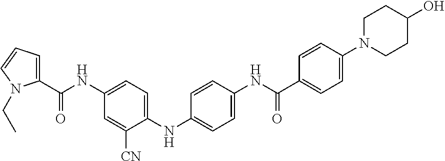 [SMILES: CCn1cccc1C(=O)Nc1ccc(Nc2ccc(NC(=O)c3ccc(N4CCC(O)CC4)cc3)cc2)c(C#N)c1]

Compound 868 was prepared according to the procedure described in Scheme IV from N¹-(4-aminophenyl)-2-cyanobenzene-1,4-diamine, 1-ethyl-1H-pyrrole-2-carboxylic, and 4-(4-hydroxypiperidin-1-yl)benzoic acids. [M+H]⁺ calcd for C₃₂H₃₂N₆O₃: 549.25. Found: 549.08.

### Example 769

### N-(3-Cyano-4-((4-(4-(4-hydroxypiperidin-1-yl)benzamido)phenyl)amino)phenyl)-1H-pyrrole-3-carboxamide (Compound 869)

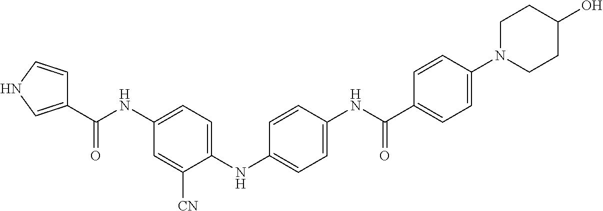 [SMILES: N#Cc1cc(NC(=O)c2cc[nH]c2)ccc1Nc1ccc(NC(=O)c2ccc(N3CCC(O)CC3)cc2)cc1]

Compound 869 was prepared according to the procedure described in Scheme IV from N¹-(4-aminophenyl)-2-cyanobenzene-1,4-diamine, 1H-pyrrole-3-carboxylic, and 4-(4-hydroxypiperidin-1-yl)benzoic acids. [M+H]⁺ calcd for C₃₀H₂₈N₆O₃: 521.22. Found: 521.00.

### Example 770

### N-(3-Fluoro-4-((4-(4-(4-hydroxypiperidin-1-yl)benzamido)phenyl)amino)phenyl)-2,4-dimethyl-1H-pyrrole-3-carboxamide (Compound 870)

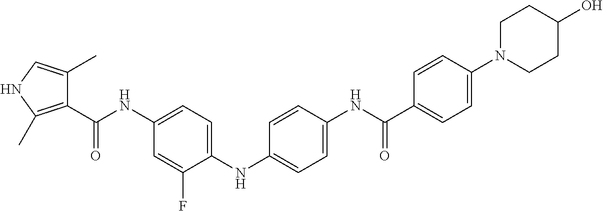 [SMILES: Cc1c[nH]c(C)c1C(=O)Nc1ccc(Nc2ccc(NC(=O)c3ccc(N4CCC(O)CC4)cc3)cc2)c(F)c1]

Compound 870 was prepared according to the procedure described in Scheme IV from N¹-(4-aminophenyl)-2-fluorobenzene-1,4-diamine, 2,4-dimethyl-1H-pyrrole-3-carboxylic, and 4-(4-hydroxypiperidin-1-yl)benzoic acids. [M+H]⁺ calcd for C₃₁H₃₂FN₅O₃: 542.25. Found 542.06.

### Example 771

### N-(3-Chloro-4-((4-(4-(4-hydroxypiperidin-1-yl)benzamido)phenyl)amino)phenyl)-1H-pyrrole-3-carboxamide (Compound 871)

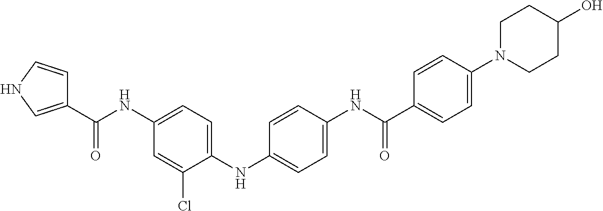 [SMILES: O=C(Nc1ccc(Nc2ccc(NC(=O)c3cc[nH]c3)cc2Cl)cc1)c1ccc(N2CCC(O)CC2)cc1]

Compound 871 was prepared according to the procedure described in Scheme IV from N¹-(4-aminophenyl)-2-chlorobenzene-1,4-diamine, 1H-pyrrole-3-carboxylic, and 4-(4-hydroxypiperidin-1-yl)benzoic acids. [M+H]⁺ calcd for C₂₉H₂₈ClN₅O₃: 530.19. Found 529.97.

### Example 772

### N-(3-Chloro-4-((4-(4-(4-hydroxypiperidin-1-yl)benzamido)phenyl)amino)phenyl)-1-methyl-1H-pyrrole-3-carboxamide (Compound 872)

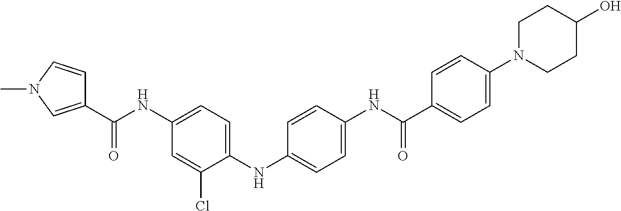 [SMILES: Cn1ccc(C(=O)Nc2ccc(Nc3ccc(NC(=O)c4ccc(N5CCC(O)CC5)cc4)cc3)c(Cl)c2)c1]

Compound 872 was prepared according to the procedure described in Scheme IV from N¹-(4-aminophenyl)-2-chlorobenzene-1,4-diamine, 1-methyl-1H-pyrrole-3-carboxylic, and 4-(4-hydroxypiperidin-1-yl)benzoic acids. [M+H]⁺ calcd for C₃₀H₃₀ClN₅O₃: 544.20. Found 544.02.

### Example 773

### N-(3-Chloro-4-((4-(4-(4-hydroxypiperidin-1-yl)benzamido)phenyl)amino)phenyl)-1-methyl-1H-pyrazole-4-carboxamide (Compound 873)

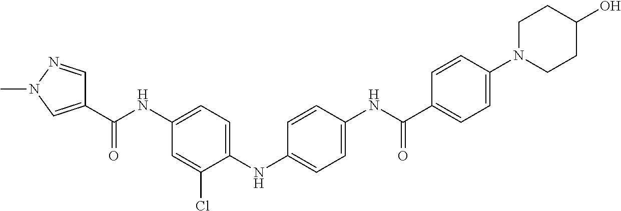 [SMILES: Cn1cc(C(=O)Nc2ccc(Nc3ccc(NC(=O)c4ccc(N5CCC(O)CC5)cc4)cc3)c(Cl)c2)cn1]

Compound 873 was prepared according to the procedure described in Scheme IV from N¹-(4-aminophenyl)-2-chlorobenzene-1,4-diamine, 1-methyl-1H-pyrazole-4-carboxylic, and 4-(4-hydroxypiperidin-1-yl)benzoic acids. [M+H]⁺ calcd for C₂₉H₂₉ClN₆O₃: 545.20. Found 544.96.

### Example 774

### N-(3-Fluoro-4-((4-(4-(4-hydroxypiperidin-1-yl)benzamido)phenyl)amino)phenyl)-1-methyl-1H-pyrazole-5-carboxamide (Compound 874)

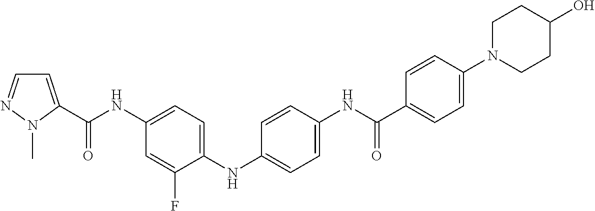 [SMILES: Cn1nccc1C(=O)Nc1ccc(Nc2ccc(NC(=O)c3ccc(N4CCC(O)CC4)cc3)cc2)c(F)c1]

Compound 874 was prepared according to the procedure described in Scheme IV from N¹-(4-aminophenyl)-2-fluorobenzene-1,4-diamine, 1-methyl-1H-pyrazole-5-carboxylic, and 4-(4-hydroxypiperidin-1-yl)benzoic acids. [M+H]⁺ calcd for C₂₉H₂₉FN₆O₃: 529.23. Found 529.03.

### Example 775

### N-(3-Chloro-4-((4-(4-(4-hydroxypiperidin-1-yl)benzamido)phenyl)amino)phenyl)-1-methyl-1H-pyrazole-5-carboxamide (Compound 875)

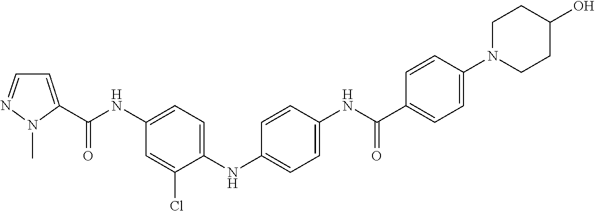 [SMILES: Cn1nccc1C(=O)Nc1ccc(Nc2ccc(NC(=O)c3ccc(N4CCC(O)CC4)cc3)cc2)c(Cl)c1]

Compound 875 was prepared according to the procedure described in Scheme IV from N¹-(4-aminophenyl)-2-chlorobenzene-1,4-diamine, 1-methyl-1H-pyrazole-5-carboxylic, and 4-(4-hydroxypiperidin-1-yl)benzoic acids. [M+H]⁺ calcd for C₂₉H₂₉ClN₆O₃: 545.20. Found 544.96.

### Example 776

### N-(3-Fluoro-4-((4-(4-(4-hydroxypiperidin-1-yl)benzamido)phenyl)amino)phenyl)-1-methyl-1H-pyrazole-4-carboxamide (Compound 876)

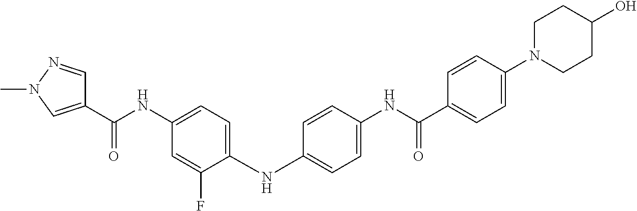 [SMILES: Cn1cc(C(=O)Nc2ccc(Nc3ccc(NC(=O)c4ccc(N5CCC(O)CC5)cc4)cc3)c(F)c2)cn1]

Compound 876 was prepared according to the procedure described in Scheme IV from N¹-(4-aminophenyl)-2-fluorobenzene-1,4-diamine, 1-methyl-1H-pyrazole-4-carboxylic, and 4-(4-hydroxypiperidin-1-yl)benzoic acids. [M+H]⁺ calcd for C₂₉H₂₉FN₆O₃: 529.23. Found 529.03.

### Example 777

### N-(4-((4-(4-(4-Hydroxypiperidin-1-yl)benzamido)-2-methylphenyl)amino)phenyl)-1-methyl-1H-pyrrole-2-carboxamide (Compound 877)

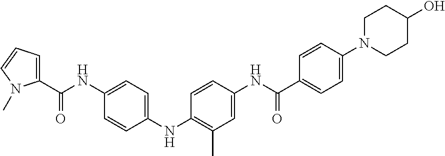 [SMILES: Cc1cc(NC(=O)c2ccc(N3CCC(O)CC3)cc2)ccc1Nc1ccc(NC(=O)c2cccn2C)cc1]

Compound 877 was prepared according to the procedure described in Scheme IV from N¹-(4-aminophenyl)-2-methylbenzene-1,4-diamine, 1-methyl-1H-pyrrole-2-carboxylic, and 4-(4-hydroxypiperidin-1-yl)benzoic acids. [M+H]⁺ calcd for C₃₁H₃₃N₅O₃: 524.26. Found 524.03.

### Example 778

### N-(4-((2-Cyano-4-(4-(4-hydroxypiperidin-1-yl)benzamido)phenyl)amino)phenyl)-1H-pyrrole-3-carboxamide (Compound 878)

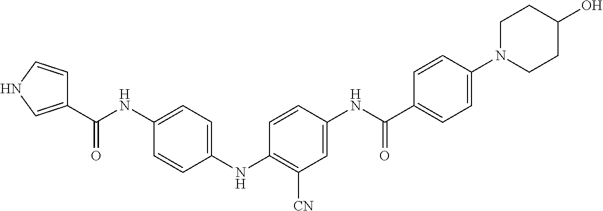 [SMILES: N#Cc1cc(NC(=O)c2ccc(N3CCC(O)CC3)cc2)ccc1Nc1ccc(NC(=O)c2cc[nH]c2)cc1]

Compound 878 was prepared according to the procedure described in Scheme IV from N¹-(4-aminophenyl)-2-cyanobenzene-1,4-diamine, 1H-pyrrole-3-carboxylic, and 4-(4-hydroxypiperidin-1-yl)benzoic acids. [M+H]⁺ calcd for C₃₀H₂₈N₆O₃: 521.22. Found: 521.00.

### Example 779

### N-(3-Chloro-4-((4-(4-(4-hydroxypiperidin-1-yl)benzamido)phenyl)amino)phenyl)-1-ethyl-1H-pyrrole-2-carboxamide (Compound 879)

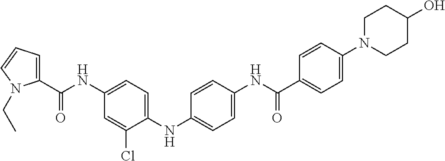 [SMILES: CCn1cccc1C(=O)Nc1ccc(Nc2ccc(NC(=O)c3ccc(N4CCC(O)CC4)cc3)cc2)c(Cl)c1]

Compound 879 was prepared according to the procedure described in Scheme IV from N¹-(4-aminophenyl)-2-chlorobenzene-1,4-diamine, 1-ethyl-1H-pyrrole-2-carboxylic, and 4-(4-hydroxypiperidin-1-yl)benzoic acids. [M+H]⁺ calcd for C₃₁H₃₂ClN₅O₃: 558.22. Found 558.06.

### Example 780

### N-(3-Chloro-4-((4-(4-(4-hydroxypiperidin-1-yl)benzamido)phenyl)amino)phenyl)-1H-indole-5-carboxamide (Compound 880)

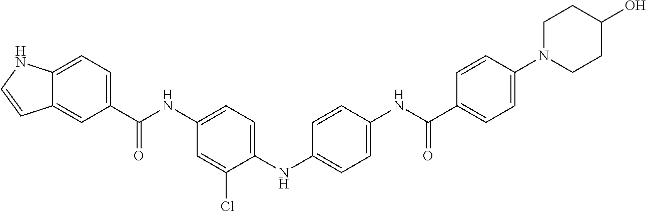 [SMILES: O=C(Nc1ccc(Nc2ccc(NC(=O)c3ccc4[nH]ccc4c3)cc2Cl)cc1)c1ccc(N2CCC(O)CC2)cc1]

Compound 880 was prepared according to the procedure described in Scheme IV from N¹-(4-aminophenyl)-2-chlorobenzene-1,4-diamine, 1H-indole-5-carboxylic, and 4-(4-hydroxypiperidin-1-yl)benzoic acids. [M+H]⁺ calcd for C₃₃H₃₀ClN₅O₃: 580.20. Found 580.00.

### Example 781

### N-(3-Fluoro-4-((4-(4-(4-ethoxypiperidin-1-yl)benzamido)phenyl)amino)phenyl)-1H-pyrrole-3-carboxamide (Compound 881)

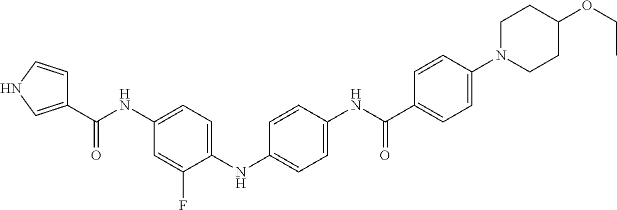 [SMILES: CCOC1CCN(c2ccc(C(=O)Nc3ccc(Nc4ccc(NC(=O)c5cc[nH]c5)cc4F)cc3)cc2)CC1]

Compound 881 was prepared according to the procedure described in Scheme IV from N¹-(4-aminophenyl)-2-fluorobenzene-1,4-diamine, 1H-pyrrole-3-carboxylic, and 4-(4-ethoxypiperidin-1-yl)benzoic acids. [M+H]⁺ calcd for C₃₁H₃₂FN₅O₃: 542.25. Found: 542.06.

### Example 782

### N-(3-Fluoro-4-((4-(4-(4-ethoxypiperidin-1-yl)benzamido)phenyl)amino)phenyl)-1-methyl-1H-pyrazole-5-carboxamide (Compound 882)

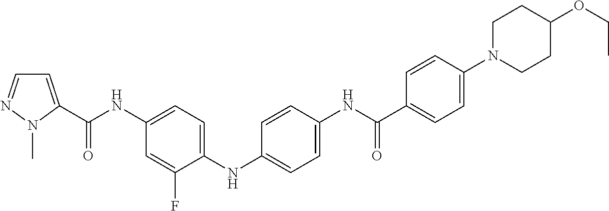 [SMILES: CCOC1CCN(c2ccc(C(=O)Nc3ccc(Nc4ccc(NC(=O)c5ccnn5C)cc4F)cc3)cc2)CC1]

Compound 882 was prepared according to the procedure described in Scheme IV from N¹-(4-aminophenyl)-2-fluorobenzene-1,4-diamine, 1-methyl-1H-pyrazole-5-carboxylic, and 4-(4-ethoxypiperidin-1-yl)benzoic acids. [M+H]⁺ calcd for C₃₁H₃₃FN₆O₃: 557.26. Found: 557.11.

### Example 783

### N-(4-((5-(4-(4-Hydroxypiperidin-1-yl)benzamido)pyridin-2-yl)amino)phenyl)-1-methyl-1H-pyrrole-3-carboxamide (Compound 883)

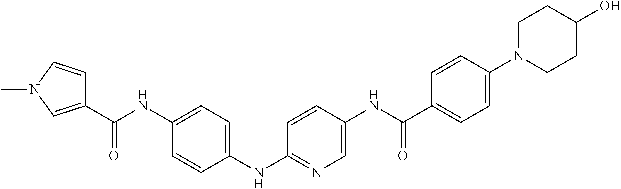 [SMILES: Cn1ccc(C(=O)Nc2ccc(Nc3ccc(NC(=O)c4ccc(N5CCC(O)CC5)cc4)cn3)cc2)c1]

Compound 883 was prepared according to the procedure described in Scheme IV from N²-(4-aminophenyl)pyridine-2,5-diamine, 1-methyl-1H-pyrrole-3-carboxylic, and 4-(4-hydroxypiperidin-1-yl)benzoic acids. [M+H]⁺ calcd for C₂₉H₃₀N₆O₃: 511.24. Found 511.01.

### Example 784

### N-(6-((4-(4-(4-Hydroxypiperidin-1-yl)benzamido)phenyl)amino)pyridin-3-yl)-1-methyl-1H-pyrrole-3-carboxamide (Compound 884)

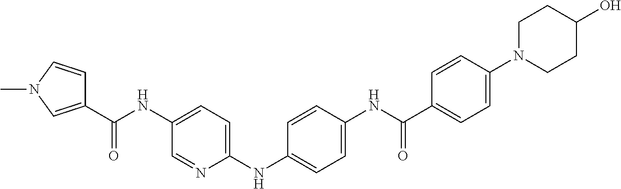 [SMILES: Cn1ccc(C(=O)Nc2ccc(Nc3ccc(NC(=O)c4ccc(N5CCC(O)CC5)cc4)cc3)nc2)c1]

Compound 884 was prepared according to the procedure described in Scheme IV from N²-(4-aminophenyl)pyridine-2,5-diamine, 1-methyl-1H-pyrrole-3-carboxylic, and 4-(4-hydroxypiperidin-1-yl)benzoic acids. [M+H]⁺ calcd for C₂₉H₃₀N₆O₃: 511.24. Found 511.01.

### Example 785

### N-(4-((2-Cyano-4-(4-(4-trifluoromethylpiperidin-1-yl)benzamido)phenyl)amino)phenyl)-1H-pyrrole-3-carboxamide (Compound 885)

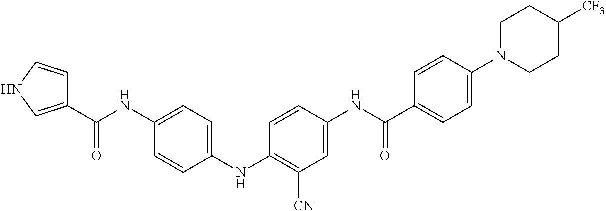 [SMILES: N#Cc1cc(NC(=O)c2ccc(N3CCC(C(F)(F)F)CC3)cc2)ccc1Nc1ccc(NC(=O)c2cc[nH]c2)cc1]

Compound 885 was prepared according to the procedure described in Scheme IV from N¹-(4-aminophenyl)-2-cyanobenzene-1,4-diamine, 1H-pyrrole-3-carboxylic, and 4-(4-trifluoromethylpiperidin-1-yl)benzoic acids. [M+H]⁺ calcd for C₃₁H₂₇F₃N₆O₂: 573.21. Found: 573.05.

### Example 786

### N-(3-Fluoro-4-((4-(4-(4-hydroxypiperidin-1-yl)benzamido)phenyl)amino)phenyl)-4-methyl-1H-pyrrole-3-carboxamide (Compound 886)

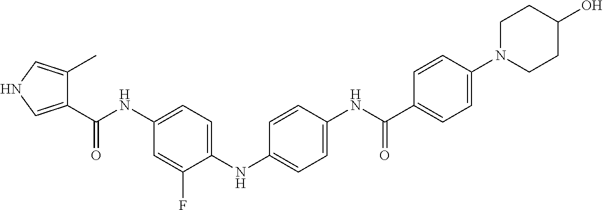 [SMILES: Cc1c[nH]cc1C(=O)Nc1ccc(Nc2ccc(NC(=O)c3ccc(N4CCC(O)CC4)cc3)cc2)c(F)c1]

Compound 886 was prepared according to the procedure described in Scheme IV from N¹-(4-aminophenyl)-2-fluorobenzene-1,4-diamine, 4-methyl-1H-pyrrole-3-carboxylic, and 4-(4-hydroxypiperidin-1-yl)benzoic acids. [M+H]⁺ calcd for C₃₀H₃₀FN₅O₃: 528.24. Found: 528.02.

### Example 787

### N-(3-Fluoro-4-((4-(4-(4-hydroxypiperidin-1-yl)benzamido)phenyl)amino)phenyl)-5-methyl-1H-pyrrole-3-carboxamide (Compound 887)

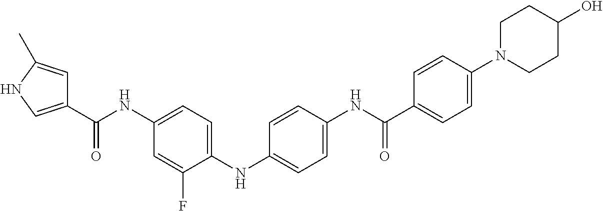 [SMILES: Cc1cc(C(=O)Nc2ccc(Nc3ccc(NC(=O)c4ccc(N5CCC(O)CC5)cc4)cc3)c(F)c2)c[nH]1]

Compound 887 was prepared according to the procedure described in Scheme IV from N¹-(4-aminophenyl)-2-fluorobenzene-1,4-diamine, 5-methyl-1H-pyrrole-3-carboxylic, and 4-(4-hydroxypiperidin-1-yl)benzoic acids. [M+H]⁺ calcd for C₃₀H₃₀FN₅O₃: 528.24. Found: 528.08.

### Example 788

### N-(3-Fluoro-4-((4-(4-(4-hydroxypiperidin-1-yl)benzamido)phenyl)amino)phenyl)-1H-indole-5-carboxamide (Compound 888)

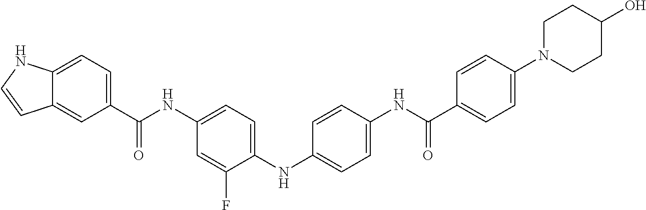 [SMILES: O=C(Nc1ccc(Nc2ccc(NC(=O)c3ccc4[nH]ccc4c3)cc2F)cc1)c1ccc(N2CCC(O)CC2)cc1]

Compound 888 was prepared according to the procedure described in Scheme IV from N¹-(4-aminophenyl)-2-fluorobenzene-1,4-diamine, 1H-indole-5-carboxylic, and 4-(4-hydroxypiperidin-1-yl)benzoic acids. [M+H]⁺ calcd for C₃₃H₃₀FN₅O₃: 564.23. Found 564.07.

### Example 789

### N-(4-((2-Cyano-4-(4-(4-hydroxypiperidin-1-yl)benzamido)phenyl)amino)phenyl)-1H-indole-5-carboxamide (Compound 889)

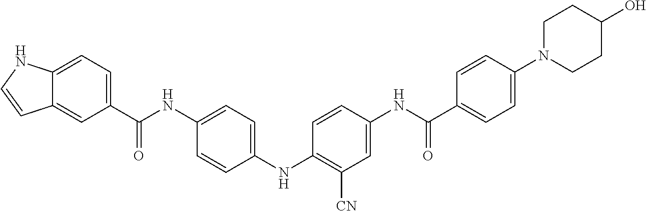 [SMILES: N#Cc1cc(NC(=O)c2ccc(N3CCC(O)CC3)cc2)ccc1Nc1ccc(NC(=O)c2ccc3[nH]ccc3c2)cc1]

Compound 889 was prepared according to the procedure described in Scheme IV from N¹-(4-aminophenyl)-2-cyanobenzene-1,4-diamine, 1H-indole-5-carboxylic, and 4-(4-hydroxypiperidin-1-yl)benzoic acids. [M+H]⁺ calcd for C₃₄H₃₀N₆O₃: 571.25. Found: 571.02.

### Example 790

### N-(4-((2-Cyano-4-(4-(4-hydroxypiperidin-1-yl)benzamido)phenyl)amino)phenyl)-1-methyl-1H-pyrazole-5-carboxamide (Compound 890)

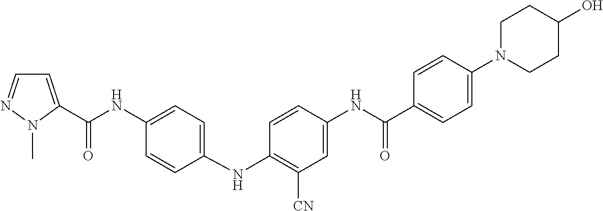 [SMILES: Cn1nccc1C(=O)Nc1ccc(Nc2ccc(NC(=O)c3ccc(N4CCC(O)CC4)cc3)cc2C#N)cc1]

Compound 890 was prepared according to the procedure described in Scheme IV from N¹-(4-aminophenyl)-2-cyanobenzene-1,4-diamine, 1-methyl-1H-pyrazole-5-carboxylic, and 4-(4-hydroxypiperidin-1-yl)benzoic acids. [M+H]⁺ calcd for C₃₀H₂₉N₇O₃: 536.24. Found: 536.05.

### Example 791

### N-(4-((4-(4-(4-Hydroxypiperidin-1-yl)benzamido)phenyl)amino)phenyl)-1,3-dimethyl-1H-pyrazole-4-carboxamide (Compound 891)

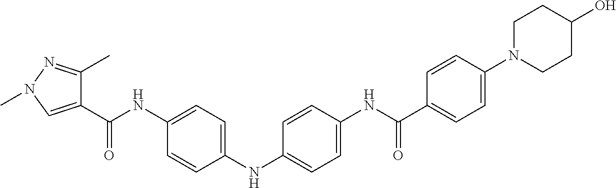 [SMILES: Cc1nn(C)cc1C(=O)Nc1ccc(Nc2ccc(NC(=O)c3ccc(N4CCC(O)CC4)cc3)cc2)cc1]

Compound 891 was prepared according to the procedure described in Scheme IV from 4,4′-diaminodiphenylamine, 1,3-dimethyl-1H-pyrazole-4-carboxylic, and 4-(4-hydroxypiperidin-1-yl)benzoic acids. [M+H]⁺ calcd for C₃₀H₃₂N₆O₃: 525.25. Found 525.05.

### Example 792

### N-(3-Fluoro-4-((4-(4-(4-hydroxypiperidin-1-yl)benzamido)phenyl)amino)phenyl)-1,3-dimethyl-1H-pyrazole-4-carboxamide (Compound 892)

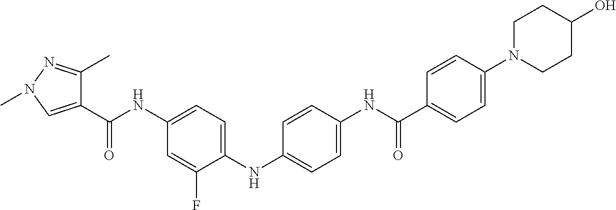 [SMILES: Cc1nn(C)cc1C(=O)Nc1ccc(Nc2ccc(NC(=O)c3ccc(N4CCC(O)CC4)cc3)cc2)c(F)c1]

Compound 892 was prepared according to the procedure described in Scheme IV from N¹-(4-aminophenyl)-2-fluorobenzene-1,4-diamine, 1,3-dimethyl-1H-pyrazole-4-carboxylic, and 4-(4-hydroxypiperidin-1-yl)benzoic acids. [M+H]⁺ calcd for C₃₀H₃₁FN₆O₃: 543.24. Found 543.07.

### Example 793

### N-(4-((4-(4-(4-Hydroxypiperidin-1-yl)benzamido)phenyl)amino)phenyl)-1,5-dimethyl-1H-pyrazole-4-carboxamide (Compound 893)

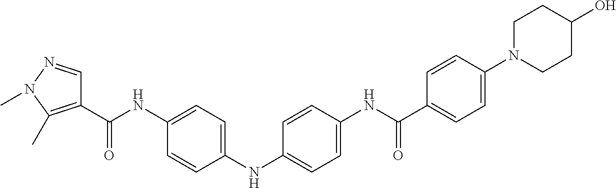 [SMILES: Cc1c(C(=O)Nc2ccc(Nc3ccc(NC(=O)c4ccc(N5CCC(O)CC5)cc4)cc3)cc2)cnn1C]

Compound 893 was prepared according to the procedure described in Scheme IV from 4,4′-diaminodiphenylamine, 1,5-dimethyl-1H-pyrazole-4-carboxylic, and 4-(4-hydroxypiperidin-1-yl)benzoic acids. [M+H]⁺ calcd for C₃₀H₃₂N₆O₃: 525.25. Found 525.05.

### Example 794

### N-(3-Fluoro-4-((4-(4-(4-hydroxypiperidin-1-yl)benzamido)phenyl)amino)phenyl)-1,5-dimethyl-1H-pyrazole-4-carboxamide (Compound 894)

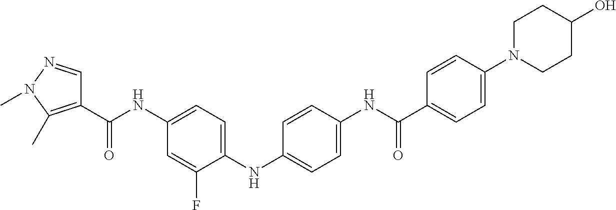 [SMILES: Cc1c(C(=O)Nc2ccc(Nc3ccc(NC(=O)c4ccc(N5CCC(O)CC5)cc4)cc3)c(F)c2)cnn1C]

Compound 894 was prepared according to the procedure described in Scheme IV from N¹-(4-aminophenyl)-2-fluorobenzene-1,4-diamine, 1,5-dimethyl-1H-pyrazole-4-carboxylic, and 4-(4-hydroxypiperidin-1-yl)benzoic acids. [M+H]⁺ calcd for C₃₀H₃₁FN₆O₃: 543.24. Found 543.07.

### Example 795

### N-(3-Chloro-4-((4-(4-(4-hydroxypiperidin-1-yl)benzamido)phenyl)amino)phenyl)-1,5-dimethyl-1H-pyrazole-4-carboxamide (Compound 895)

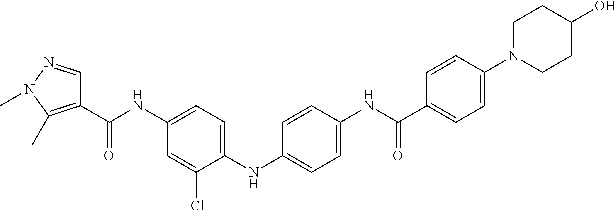 [SMILES: Cc1c(C(=O)Nc2ccc(Nc3ccc(NC(=O)c4ccc(N5CCC(O)CC5)cc4)cc3)c(Cl)c2)cnn1C]

Compound 895 was prepared according to the procedure described in Scheme IV from N¹-(4-aminophenyl)-2-chlorobenzene-1,4-diamine, 1,5-dimethyl-1H-pyrazole-4-carboxylic, and 4-(4-hydroxypiperidin-1-yl)benzoic acids. [M+H]⁺ calcd for C₃₀H₃₁ClN₆O₃: 559.21. Found 559.00.

### Example 796

### N-(3-Chloro-4-((4-(4-(4-hydroxypiperidin-1-yl)benzamido)phenyl)amino)phenyl)-1,3-dimethyl-1H-pyrazole-4-carboxamide (Compound 896)

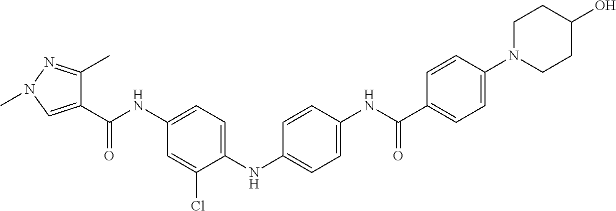 [SMILES: Cc1nn(C)cc1C(=O)Nc1ccc(Nc2ccc(NC(=O)c3ccc(N4CCC(O)CC4)cc3)cc2)c(Cl)c1]

Compound 896 was prepared according to the procedure described in Scheme IV from N¹-(4-aminophenyl)-2-chlorobenzene-1,4-diamine, 1,3-dimethyl-1H-pyrazole-4-carboxylic, and 4-(4-hydroxypiperidin-1-yl)benzoic acids. [M+H]⁺ calcd for C₃₀H₃₁ClN₆O₃: 559.21. Found 559.00.

### Example 797

### N-(4-((4-(4-(4-Hydroxypiperidin-1-yl)benzamido)phenyl)amino)phenyl)-3,5-dimethyl-1H-pyrazole-4-carboxamide (Compound 897)

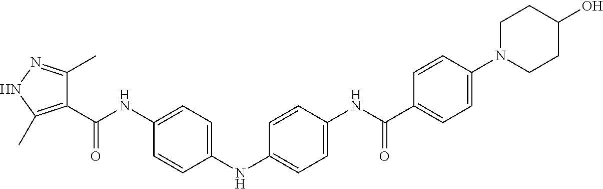 [SMILES: Cc1n[nH]c(C)c1C(=O)Nc1ccc(Nc2ccc(NC(=O)c3ccc(N4CCC(O)CC4)cc3)cc2)cc1]

Compound 897 was prepared according to the procedure described in Scheme IV from 4,4′-diaminodiphenylamine, 3,5-dimethyl-1H-pyrazole-4-carboxylic, and 4-(4-hydroxypiperidin-1-yl)benzoic acids. [M+H]⁺ calcd for C₃₀H₃₂N₆O₃: 525.25. Found 525.05.

### Example 798

### N-(4-((2-Fluoro-4-(4-(4-hydroxypiperidin-1-yl)benzamido)phenyl)amino)phenyl)-1-methyl-1H-pyrazole-5-carboxamide (Compound 898)

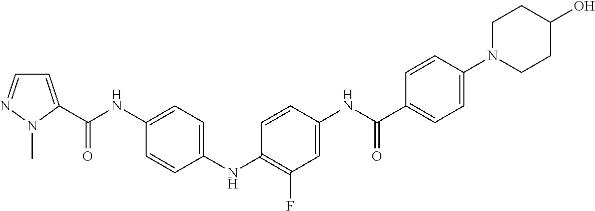 [SMILES: Cn1nccc1C(=O)Nc1ccc(Nc2ccc(NC(=O)c3ccc(N4CCC(O)CC4)cc3)cc2F)cc1]

Compound 898 was prepared according to the procedure described in Scheme IV from N¹-(4-aminophenyl)-2-fluorobenzene-1,4-diamine, 1-methyl-1H-pyrazole-5-carboxylic, and 4-(4-hydroxypiperidin-1-yl)benzoic acids. [M+H]⁺ calcd for C₂₉H₂₉FN₆O₃: 529.23. Found: 529.03.

### Example 799

### N-(4-((4-(4-(4-Hydroxypiperidin-1-yl)benzamido)phenyl)amino)phenyl)-1,4,5,6-tetrahydrocyclopenta[c]pyrazole-3-carboxamide (Compound 899)

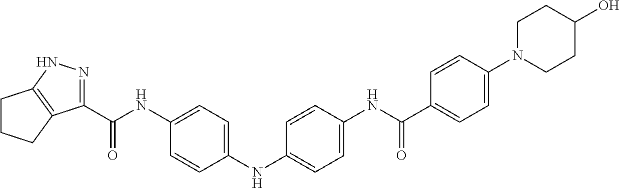 [SMILES: O=C(Nc1ccc(Nc2ccc(NC(=O)c3n[nH]c4c3CCC4)cc2)cc1)c1ccc(N2CCC(O)CC2)cc1]

Compound 899 was prepared according to the procedure described in Scheme IV from 4,4′-diaminodiphenylamine, 1,4,5,6-tetrahydrocyclopenta[c]pyrazole-3-carboxylic, and 4-(4-hydroxypiperidin-1-yl)benzoic acids. [M+H]⁺ calcd for C₃₁H₃₂N₆O₃: 537.25. Found 537.06.

### Example 800

### N-(4-((4-(4-(4-Hydroxypiperidin-1-yl)benzamido)phenyl)amino)phenyl)-1-ethyl-5-methyl-1H-pyrrole-2-carboxamide (Compound 900)

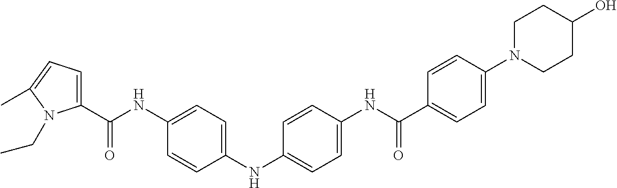 [SMILES: CCn1c(C)ccc1C(=O)Nc1ccc(Nc2ccc(NC(=O)c3ccc(N4CCC(O)CC4)cc3)cc2)cc1]

Compound 900 was prepared according to the procedure described in Scheme IV from 4,4′-diaminodiphenylamine, 1-ethyl-5-methyl-1H-pyrrole-2-carboxylic, and 4-(4-hydroxypiperidin-1-yl)benzoic acids. [M+H]⁺ calcd for C₃₂H₃₅N₅O₃: 538.27. Found: 538.08.

### Example 801

### N-(4-((4-(4-(4-Hydroxypiperidin-1-yl)benzamido)phenyl)amino)phenyl)-5-hydroxymethyl-1H-pyrrole-2-carboxamide (Compound 901)

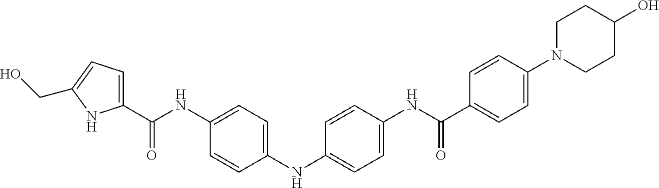 [SMILES: O=C(Nc1ccc(Nc2ccc(NC(=O)c3ccc(CO)[nH]3)cc2)cc1)c1ccc(N2CCC(O)CC2)cc1]

Compound 901 was prepared according to the procedure described in Scheme IV from 4,4′-diaminodiphenylamine, 5-hydroxymethyl-1H-pyrrole-2-carboxylic, and 4-(4-hydroxypiperidin-1-yl)benzoic acids. [M+H]⁺ calcd for C₃₂H₃₃N₅O₅: 568.16. Found: 568.05.

### Example 802

### Methyl 4-((4-((4-(4-(4-hydroxypiperidin-1-yl)benzamido)phenyl)amino)phenyl)carbamoyl)-1H-pyrrole-2-carboxylate (Compound 902)

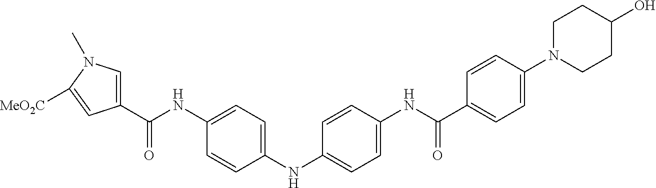 [SMILES: COC(=O)c1cc(C(=O)Nc2ccc(Nc3ccc(NC(=O)c4ccc(N5CCC(O)CC5)cc4)cc3)cc2)cn1C]

Compound 902 was prepared according to the procedure described in Scheme IV from 4,4′-diaminodiphenylamine, 5-methoxycarbonyl-1H-pyrrole-3-carboxylic, and 4-(4-hydroxypiperidin-1-yl)benzoic acids. [M+H]⁺ calcd for C₃₀H₃₁N₅O₄: 526.12. Found: 526.06.

### Example 803

### N-(3-Fluoro-4-((2-fluoro-4-(4-(4-hydroxypiperidin-1-yl)benzamido)phenyl)amino)phenyl)-1H-pyrrole-2-carboxamide (Compound 903)

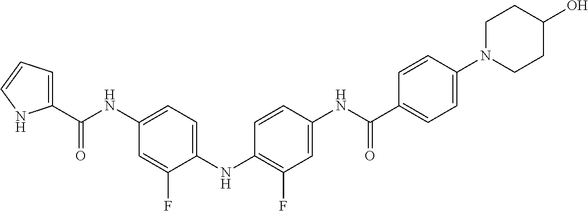 [SMILES: O=C(Nc1ccc(Nc2ccc(NC(=O)c3ccc[nH]3)cc2F)c(F)c1)c1ccc(N2CCC(O)CC2)cc1]

Compound 903 was prepared according to the procedure described in Scheme IV from 4,4′-diamino-2,2′-difluorodiphenylamine, 1H-pyrrole-2-carboxylic, and 4-(4-hydroxypiperidin-1-yl)benzoic acids. [M+H]⁺ calcd for C₂₉H₂₇F₂N₅O₃: 532.21. Found: 532.00.

### Example 804

### N-(3-Fluoro-4-((2-fluoro-4-(4-(4-hydroxypiperidin-1-yl)benzamido)phenyl)amino)phenyl)-1-methyl-1H-pyrazole-5-carboxamide (Compound 904)

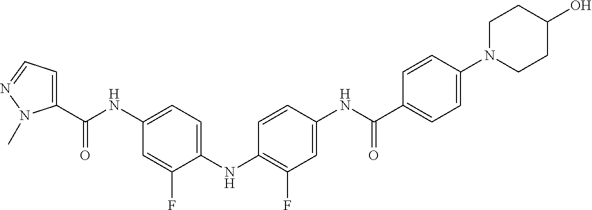 [SMILES: Cn1nccc1C(=O)Nc1ccc(Nc2ccc(NC(=O)c3ccc(N4CCC(O)CC4)cc3)cc2F)c(F)c1]

Compound 904 was prepared according to the procedure described in Scheme IV from 4,4′-diamino-2,2′-difluorodiphenylamine, 1-methyl-1H-pyrazole-5-carboxylic, and 4-(4-hydroxypiperidin-1-yl)benzoic acids. [M+H]⁺ calcd for C₂₉H₂₈F2N₆O₃: 547.22. Found: 547.05.

### Example 805

### N-(3-Fluoro-4-((2-fluoro-4-(4-(4-hydroxypiperidin-1-yl)benzamido)phenyl)amino)phenyl)-1H-pyrrole-3-carboxamide (Compound 905)

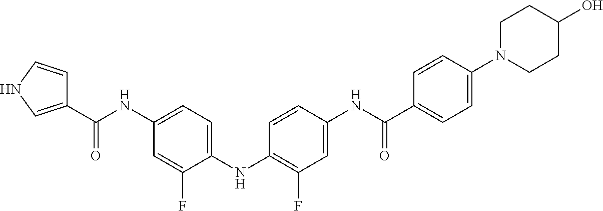 [SMILES: O=C(Nc1ccc(Nc2ccc(NC(=O)c3cc[nH]c3)cc2F)c(F)c1)c1ccc(N2CCC(O)CC2)cc1]

Compound 905 was prepared according to the procedure described in Scheme IV from 4,4′-diamino-2,2′-difluorodiphenylamine, 1H-pyrrole-3-carboxylic, and 4-(4-hydroxypiperidin-1-yl)benzoic acids. [M+H]⁺ calcd for C₂₉H₂₇F₂N₅O₃: 532.21. Found 532.00.

### Example 806

### N-(4-((2-Fluoro-4-(4-(4-hydroxypiperidin-1-yl)benzamido)phenyl)amino)phenyl)-5-methyl-1H-pyrrole-3-carboxamidee (Compound 906)

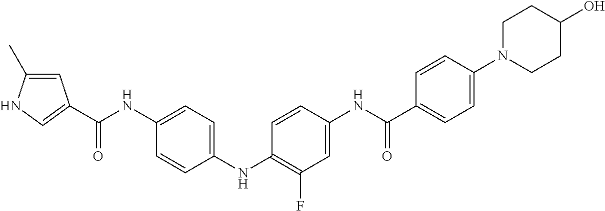 [SMILES: Cc1cc(C(=O)Nc2ccc(Nc3ccc(NC(=O)c4ccc(N5CCC(O)CC5)cc4)cc3F)cc2)c[nH]1]

Compound 906 was prepared according to the procedure described in Scheme IV from N¹-(4-aminophenyl)-2-fluorobenzene-1,4-diamine, 5-methyl-1H-pyrrole-3-carboxylic, and 4-(4-hydroxypiperidin-1-yl)benzoic acids. [M+H]⁺ calcd for C₃₀H₃₀FN₅O₃: 528.23. Found: 528.02.

### Example 807

### N-(4-((2-Fluoro-4-(4-(4-hydroxypiperidin-1-yl)benzamido)phenyl)amino)phenyl)-4-methyl-1H-pyrrole-3-carboxamidee (Compound 907)

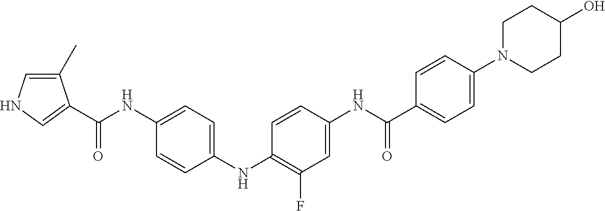 [SMILES: Cc1c[nH]cc1C(=O)Nc1ccc(Nc2ccc(NC(=O)c3ccc(N4CCC(O)CC4)cc3)cc2F)cc1]

Compound 907 was prepared according to the procedure described in Scheme IV from N¹-(4-aminophenyl)-2-fluorobenzene-1,4-diamine, 4-methyl-1H-pyrrole-3-carboxylic, and 4-(4-hydroxypiperidin-1-yl)benzoic acids. [M+H]⁺ calcd for C₃₀H₃₀FN₅O₃: 528.23. Found: 528.02.

### Example 808

### N-(4-((2-Chloro-4-(4-(4-hydroxypiperidin-1-yl)benzamido)phenyl)amino)phenyl)-1H-pyrrole-3-carboxamidee (Compound 908)

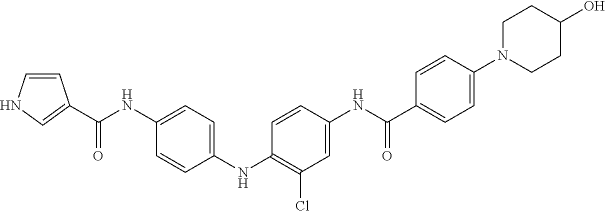 [SMILES: O=C(Nc1ccc(Nc2ccc(NC(=O)c3cc[nH]c3)cc2)c(Cl)c1)c1ccc(N2CCC(O)CC2)cc1]

Compound 908 was prepared according to the procedure described in Scheme IV from N¹-(4-aminophenyl)-2-chlorobenzene-1,4-diamine, 1H-pyrrole-3-carboxylic, and 4-(4-hydroxypiperidin-1-yl)benzoic acids. [M+H]⁺ calcd for C₂₉H₂₈ClN₅O₃: 530.19. Found 529.97.

### Example 809

### N-(4-((2-Chloro-4-(4-(4-hydroxypiperidin-1-yl)benzamido)phenyl)amino)phenyl)-1-methyl-1H-pyrrole-3-carboxamidee (Compound 909)

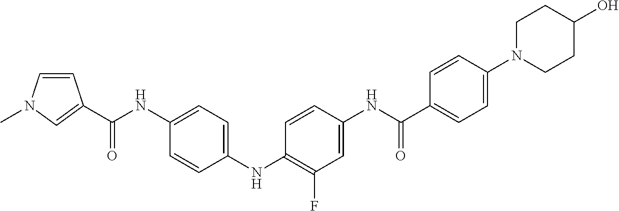 [SMILES: Cn1ccc(C(=O)Nc2ccc(Nc3ccc(NC(=O)c4ccc(N5CCC(O)CC5)cc4)cc3F)cc2)c1]

Compound 909 was prepared according to the procedure described in Scheme IV from N¹-(4-aminophenyl)-2-chlorobenzene-1,4-diamine, 1-methyl-1H-pyrrole-3-carboxylic, and 4-(4-hydroxypiperidin-1-yl)benzoic acids. [M+H]⁺ calcd for C₃₀H₃₀ClN₅O₃: 544.20. Found 544.02.

### Example 810

### N—,N′-(Azanediylbis(3-fluoro-4,1-phenylene))bis(4-(4-hydroxypiperidin-1-yl)benzamide) (Compound 910)

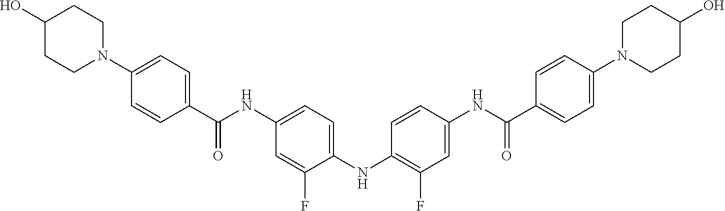 [SMILES: O=C(Nc1ccc(Nc2ccc(NC(=O)c3ccc(N4CCC(O)CC4)cc3)cc2F)c(F)c1)c1ccc(N2CCC(O)CC2)cc1]

Compound 910 was prepared according to the procedure described in Scheme IV from 4,4′-diamino-2,2′-difluorodiphenylamine and 4-(4-hydroxypiperidin-1-yl)benzoic acid. [M+H]⁺ calcd for C₃₆H₃₇F2N₅O₄: 642.28. Found 642.12

### Example 811

### N-(4-((2-Fluoro-4-(4-(4,4-difluoropiperidin-1-yl)benzamido)phenyl)amino)phenyl)-1H-pyrrole-3-carboxamidee (Compound 911)

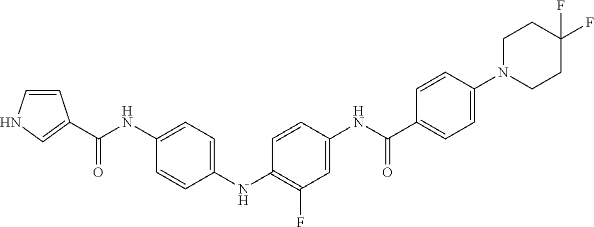 [SMILES: O=C(Nc1ccc(Nc2ccc(NC(=O)c3cc[nH]c3)cc2)c(F)c1)c1ccc(N2CCC(F)(F)CC2)cc1]

Compound 911 was prepared according to the procedure described in Scheme IV from N¹-(4-aminophenyl)-2-fluorobenzene-1,4-diamine, 1H-pyrrole-3-carboxylic, and 4-(4,4-difluoropiperidin-1-yl)benzoic acids. ¹H NMR (400 MHz, Acetone-d₆) δ 10.54 (s, 1H), 9.39 (s, 1H), 8.88 (s, 1H), 7.92 (m, 3H), 7.69 (d, J=8.8 Hz, 2H), 7.54 (m, 1H), 7.43 (d, J=8.8 Hz, 1H), 7.25 (t, J=9.2 Hz, 1H), 7.07 (t, J=8.8 Hz, 4H), 6.91 (s, 1H), 6.85 (q, J=2.3 Hz, 1H), 6.71 (q, J=1.6 Hz, 1H), 3.55 (t, J=5.8 Hz, 4H), 2.11 (m, 4H).

### Example 812

### N-(4-((2-Fluoro-4-(4-(4-hydroxypiperidin-1-yl)benzamido)phenyl)amino)phenyl)-1H-indole-5-carboxamidee (Compound 912)

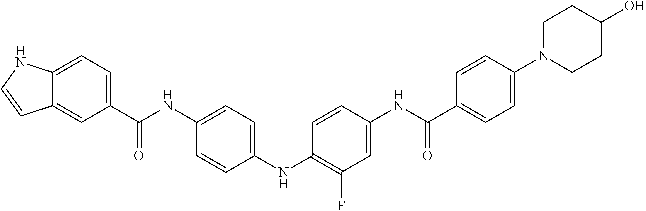 [SMILES: O=C(Nc1ccc(Nc2ccc(NC(=O)c3ccc4[nH]ccc4c3)cc2)c(F)c1)c1ccc(N2CCC(O)CC2)cc1]

Compound 912 was prepared according to the procedure described in Scheme IV from N¹-(4-aminophenyl)-2-fluorobenzene-1,4-diamine, 1H-indole-5-carboxylic, and 4-(4-hydroxypiperidin-1-yl)benzoic acids. [M+H]⁺ calcd for C₃₃H₃₀FN₅O₃: 564.23. Found 564.07.

### Example 813

### N-(3-Fluoro-4-((4-(4-(4-hydroxypiperidin-1-yl)benzamido)phenyl)amino)phenyl)-1,4,5-trimethyl-1H-pyrrole-3-carboxamide (Compound 913)

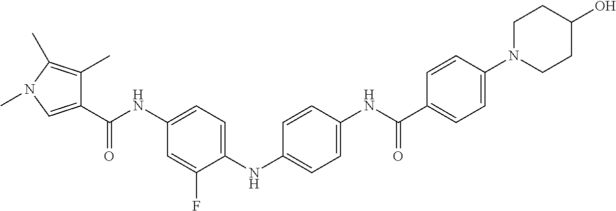 [SMILES: Cc1c(C(=O)Nc2ccc(Nc3ccc(NC(=O)c4ccc(N5CCC(O)CC5)cc4)cc3)c(F)c2)cn(C)c1C]

Compound 913 was prepared according to the procedure described in Scheme IV from N¹-(4-aminophenyl)-2-fluorobenzene-1,4-diamine, 1,4,5-trimethyl-1H-pyrrole-3-carboxylic, and 4-(4-hydroxypiperidin-1-yl)benzoic acids. [M+H]⁺ calcd for C₃₂H₃₄FN₅O₃: 556.26. Found: 556.03.

### Example 814

### N—,N′-(Oxybis(4,1-phenylene))bis(4-(4-hydroxypiperidin-1-yl)benzamide) (Compound 914)

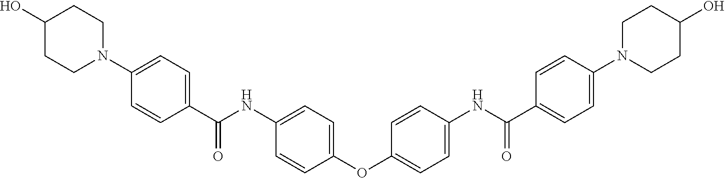 [SMILES: O=C(Nc1ccc(Oc2ccc(NC(=O)c3ccc(N4CCC(O)CC4)cc3)cc2)cc1)c1ccc(N2CCC(O)CC2)cc1]

Compound 914 was prepared according to the procedure described in Scheme IV from 4,4′-oxydianiline and 4-(4-hydroxypiperidin-1-yl)benzoic acid. [M+H]⁺ calcd for C₃₆H₃₈N₄O₅: 607.28. Found 607.21.

### Example 815

### N-(4-(4-(4-(4-Hydroxypiperidin-1-yl)benzamido)phenoxy)phenyl)-1-methyl-1H-pyrrole-2-carboxamide (Compound 915)

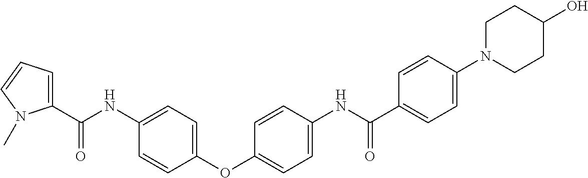 [SMILES: Cn1cccc1C(=O)Nc1ccc(Oc2ccc(NC(=O)c3ccc(N4CCC(O)CC4)cc3)cc2)cc1]

Compound 915 was prepared according to the procedure described in Scheme IV from 4,4′-oxydianiline, 1-methyl-1H-pyrrole-2-carboxylic, and 4-(4-hydroxypiperidin-1-yl)benzoic acids. [M+H]⁺ calcd for C₃₀H₃₀N₄O₄: 511.23. Found 511.01.

### Example 816

### N-(4-(4-(4-(4-Hydroxypiperidin-1-yl)benzamido)phenoxy)phenyl)-1H-pyrrole-2-carboxamide (Compound 916)

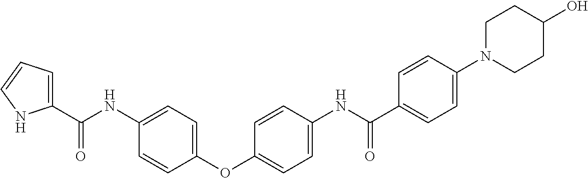 [SMILES: O=C(Nc1ccc(Oc2ccc(NC(=O)c3ccc[nH]3)cc2)cc1)c1ccc(N2CCC(O)CC2)cc1]

Compound 916 was prepared according to the procedure described in Scheme IV from 4,4′-oxydianiline, 1H-pyrrole-2-carboxylic, and 4-(4-hydroxypiperidin-1-yl)benzoic acids. [M+H]⁺ calcd for C₂₉H₂₈N₄O₄: 497.21. Found 496.97.

### Example 817

### N-(4-(4-(4-(4-Hydroxypiperidin-1-yl)benzamido)phenoxy)phenyl)-1H-indole-6-carboxamide (Compound 917).

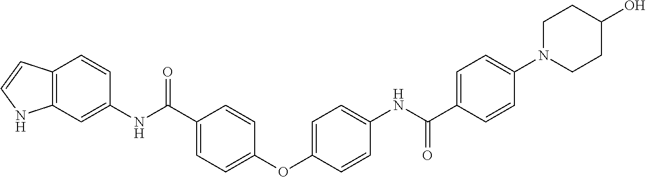 [SMILES: O=C(Nc1ccc(Oc2ccc(C(=O)Nc3ccc4cc[nH]c4c3)cc2)cc1)c1ccc(N2CCC(O)CC2)cc1]

Compound 917 was prepared according to the procedure described in Scheme IV from 4,4′-oxydianiline, 1H-indole-6-carboxylic, and 4-(4-hydroxypiperidin-1-yl)benzoic acids. [M+H]⁺ calcd for C₃₃H₃₀N₄O₄: 547.23. Found 547.05.

### Example 818

### N-(4-(4-(4-(4-Hydroxypiperidin-1-yl)benzamido)phenoxy)phenyl)-1-methyl-1H-indole-3-carboxamide (Compound 918)

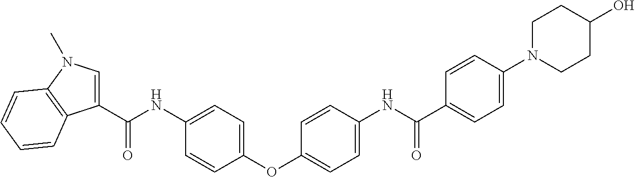 [SMILES: Cn1cc(C(=O)Nc2ccc(Oc3ccc(NC(=O)c4ccc(N5CCC(O)CC5)cc4)cc3)cc2)c2ccccc21]

Compound 918 was prepared according to the procedure described in Scheme IV from 4,4′-oxydianiline, 1-methyl-1H-indole-3-carboxylic, and 4-(4-hydroxypiperidin-1-yl)benzoic acids. [M+H]⁺ calcd for C₃₄H₃₂N₄O₄: 561.24. Found 561.10.

### Example 819

### N-(4-(4-Aminophenoxy)phenyl)-4-(4-hydroxypiperidin-1-yl)benzamide (Compound 919)

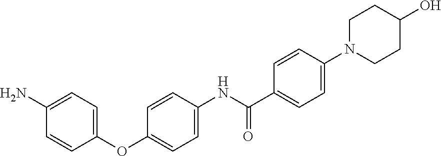 [SMILES: Nc1ccc(Oc2ccc(NC(=O)c3ccc(N4CCC(O)CC4)cc3)cc2)cc1]

Compound 919 was prepared according to the procedure described in Scheme IV from 4,4′-oxydianiline and 4-(4-hydroxypiperidin-1-yl)benzoic acid. [M+H]⁺ calcd for C₂₄H₂₅N₃O₃: 404.19. Found 404.05.

### Example 820

### N-(3-((1H-Indol-6-yl)amino)-3-oxopropyl)-4-(4-(4-hydroxypiperidin-1-yl)benzamido)benzamide (Compound 920)

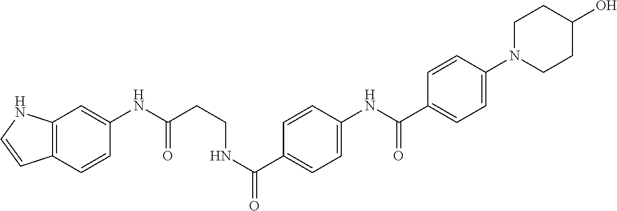 [SMILES: O=C(CCNC(=O)c1ccc(NC(=O)c2ccc(N3CCC(O)CC3)cc2)cc1)Nc1ccc2cc[nH]c2c1]

Compound 920 was prepared according to the procedure described in Scheme IV from 6-aminoindole, 3-aminopropanoic, 4-aminobenzoic, and 4-(4-hydroxypiperidin-1-yl)benzoic acid. [M+H]⁺ calcd for C₃₀H₃₁N₅O₄: 526.24. Found 525.99.

### Example 821

### N-(4-(5-(4-(4-Methoxypiperidin-1-yl)benzamido)-1H-indazol-1-yl)phenyl)thiophene-2-carboxamide (Compound 921)

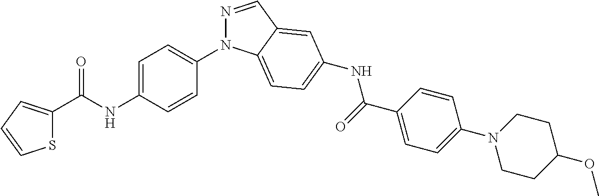 [SMILES: COC1CCN(c2ccc(C(=O)Nc3ccc4c(cnn4-c4ccc(NC(=O)c5cccs5)cc4)c3)cc2)CC1]

Compound 921 was prepared according to the general procedure described in Scheme IV. Preparation of 4-fluoro-N-(1H-indazol-5-yl)benzamide: 1H-Indazol-5-amine (5.5 g, 41 mmol), hydroxybenzotriazole (558 mg, 4.13 mmol), triethylamine (6.3 mL, 45 mmol), and 4-fluorobenzoic acid (6.36 g, 45 mmol) were taken up in DMF (207 mL) and stirred. EDC (8.7 g, 45 mmol) was added to the solution last. After the addition, the solution was stirred at room temperature for 4 h. Water was then added to the solution and stirred for 10 min. The formed precipitate was filtered and washed well with water, followed by hexanes. The solid was dried under vacuum to give 10.3 g (98%) of the product as a light purple solid.

Preparation of N-(1-(4-aminophenyl)-1H-indazol-5-yl)-4-fluorobenzamide: 4-Fluoro-N-(1H-indazol-5-yl)benzamide (10.3 g, 40 mmol), 4-fluoronitrobenze (4.3 mL, 40 mmol) and cesium carbonate (13 g, 40 mmol) were taken up in DMSO (400 mL). The solution was heated to 100° C. and stirred for 24 h. After the solution was cooled it was diluted with water until a precipitate formed and stirred well for 5 min. Filtration gave a yellow solid, which was then washed well with water, followed by hexanes. After the solid was dried under vacuum it was taken up in ethanol and stirred at RT under nitrogen. The solution was treated with Pd(OH)₂ (100 mg) and placed under a balloon of H₂ gas. After stirring at RT for 24 h, the catalyst was removed via filtration through celite. The filtrate was concentrated under reduced pressure to give 9.36 g (67%) of a crude product as a yellow solid

Preparation of N-(4-(5-(4-fluorobenzamido)-1H-indazol-1-yl)phenyl)thiophene-2-carboxamide: N-(1-(4-Aminophenyl)-1H-indazol-5-yl)-4-fluorobenzamide (400 mg, 1.15 mmol) was taken up in anhydrous pyridine (5.8 mL) and stirred at RT. Thiophene-2-carbonyl chloride (0.15 mL, 1.44 mmol) was then added dropwise and the solution was stirred at RT for 4 h. Added water, EtOAc and hexanes to the solution and stirred vigorously for 1 h. to form a precipitate. Filtered off the solid and dried under vacuum to obtain 539 mg (94%) of the product as an off-white solid.

Preparation of Compound 921: N-(4-(5-(4-Fluorobenzamido)-1H-indazol-1-yl)phenyl)thiophene-2-carboxamide (25 mg, 0.05 mmol), 4-methoxypiperidine (165 mg, 1.09 mmol), cesium carbonate (357 mg, 1.09 mmol) and DMSO (1 mL) were added to a microwave vial. The mixture was heated in a microwave at 130° C. for 5 h. Water was added to the cooled solution and shaken vigorously, which formed a precipitate. The solid was filtered and washed with water, followed by hexanes. Took up the solid with a CH₂Cl₂/MeOH mixture and concentrated under reduced pressure onto silica. Purification via flash chromatography (0-10% MeOH/CH₂Cl₂) produced 18.6 mg (61%) of compound 921. ¹H NMR (500 MHz, DMSO-d₆) 10.41 (s, 1H), 10.06 (s, 1H), 8.37 (s, 1H), 8.32 (s, 1H), 8.05 (d, J=5 Hz, 1H), 7.94 (d, J=9 Hz, 2H), 7.88 (d, J=9 Hz, 2H), 7.86 (d, J=1 Hz, 1H), 7.83 (d, J=9 Hz, 2H), 7.77 (d, J=9.5 Hz, 1H), 7.77 (d, J=9 Hz, 2H), 7.24 (dd, J=4, 5 Hz, 1H), 7.00 (d, J=9 Hz, 2H), 5.75 (s, 3H), 3.66-3.63 (m, 2H), 3.07-3.02 (m, 2H), 1.94-1.88 (m, 2H), 1.52-1.45 (m, 2H).

### Example 822

### N-(4-(5-(4-(4-Hydroxypiperidin-1-yl)benzamido)-1H-indazol-1-yl)phenyl)benzo[d]thiazole-5-carboxamide (Compound 922)

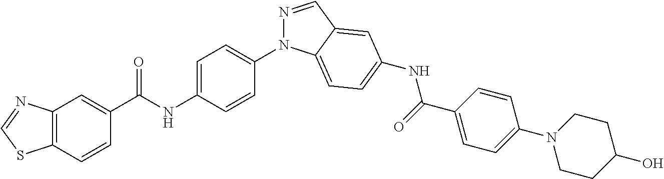 [SMILES: O=C(Nc1ccc2c(cnn2-c2ccc(NC(=O)c3ccc4scnc4c3)cc2)c1)c1ccc(N2CCC(O)CC2)cc1]

Compound 922 was prepared according to the procedure described in Scheme IV from 5-amino-1-(4-aminophenyl)indazole, benzo[d]thiazole-5-carboxylic, and 4-(4-hydroxypiperidin-1-yl)benzoic acids. [M+H]⁺ calcd for C₃₃H₂₈N₆O₃S: 589.19. Found: 589.12.

### Example 823

### N-(4-(5-(4-(4-Hydroxypiperidin-1-yl)benzamido)-1H-indazol-1-yl)phenyl)isonicotinamide (Compound 923)

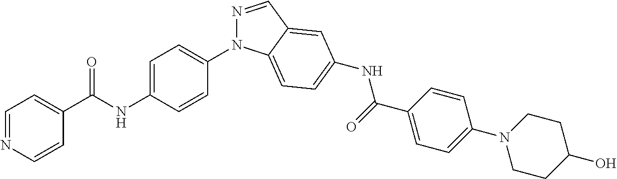 [SMILES: O=C(Nc1ccc(-n2ncc3cc(NC(=O)c4ccc(N5CCC(O)CC5)cc4)ccc32)cc1)c1ccncc1]

Compound 923 was prepared according to the procedure described in Scheme IV from 5-amino-1-(4-aminophenyl)indazole, isonictinic, and 4-(4-hydroxypiperidin-1-yl)benzoic acids. [M+H]⁺ calcd for C₃₁H₂₈N₆O₃: 533.22. Found: 533.14.

### Example 824

### N-(1-(4-(Cyclopropanecarboxamido)phenyl)-1H-indazol-5-yl)-4-(4-(2-hydroxyethyl)piperidin-1-yl)benzamide (Compound 924)

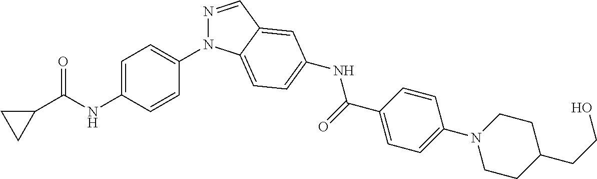 [SMILES: O=C(Nc1ccc2c(cnn2-c2ccc(NC(=O)C3CC3)cc2)c1)c1ccc(N2CCC(CCO)CC2)cc1]

Compound 924 was prepared according to the procedure described in Scheme IV from 5-amino-1-(4-aminophenyl)indazole, cyclopropanecarboxylic, and 4-(4-(2-hydroxyethyl)piperidin-1-yl)benzoic acids. [M+H]⁺ calcd for C₃₁H₃₃N₅O₃: 524.26. Found: 524.10.

### Example 825

### (±)-N-(1-(4-(Cyclopropanecarboxamido)phenyl)-1H-indazol-5-yl)-4-(3-hydroxypiperidin-1-yl)benzamide (Compound 925)

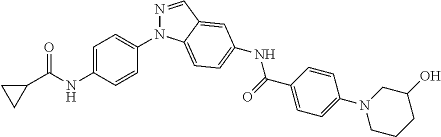 [SMILES: O=C(Nc1ccc2c(cnn2-c2ccc(NC(=O)C3CC3)cc2)c1)c1ccc(N2CCCC(O)C2)cc1]

Compound 925 was prepared according to the procedure described in Scheme IV from 5-amino-(4-aminophenyl)indazole, cyclopropanecarboxylic, and 4-(3-hydroxypiperidin-1-yl)benzoic acids. [M+H]⁺ calcd for C₂₉H₂₉N₅O₃: 496.23. Found: 496.14.

### Example 826

### N-(1-(4-(2,2-Difluorocyclopropanecarboxamido)phenyl)-1H-indazol-5-yl)-4-(4-hydroxypiperidin-1-yl)benzamide (Compound 926)

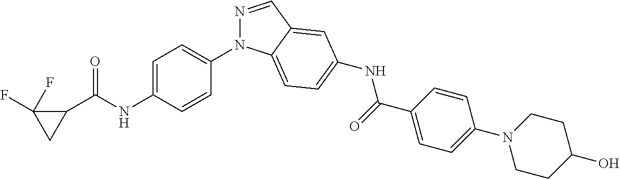 [SMILES: O=C(Nc1ccc2c(cnn2-c2ccc(NC(=O)C3CC3(F)F)cc2)c1)c1ccc(N2CCC(O)CC2)cc1]

Compound 926 was prepared according to the procedure described in Scheme IV from 5-amino-1-(4-aminophenyl)indazole, 2,2-difluorocyclopropanecarboxylic, and 4-(4-hydroxypiperidin-1-yl)benzoic acids. ¹H NMR (500 MHz, DMSO-d₆) δ 7.38 (s, 1H), 7.37 (m, 1H), 7.04 (d, J=5 Hz, 1H), 7.94 (d, J=8.5 Hz, 2H), 6.98 (d, J=9 Hz, 2H), 6.87 (d, J=11 Hz, 2H), 6.85 (d, J=9 Hz, 2H), 6.19 (d, J=9 Hz, 2H), 3.01-2.93 (m, 3H), 2.22 (ddd, J=3, 10.5, 13 Hz, 2H), 1.94-1.88 (m, 1H), 1.32-1.27 (m, 1H), 1.16-1.12 (m, 2H), 1.07-1.00 (m, 1H), 0.82-0.74 (m, 2H).

### Example 827

### N-(4-(5-(4-(4-Hydroxypiperidin-1-yl)benzamido)-1H-indazol-1-yl)phenyl)-1-methyl-1H-indole-6-carboxamide (Compound 927)

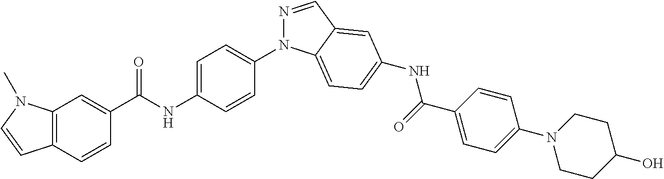 [SMILES: Cn1ccc2ccc(C(=O)Nc3ccc(-n4ncc5cc(NC(=O)c6ccc(N7CCC(O)CC7)cc6)ccc54)cc3)cc21]

Compound 927 was prepared according to the procedure described in Scheme IV from 5-amino-1-(4-aminophenyl)indazole, 1-methyl-1H-indole-6-carboxylic, and 4-(4-hydroxypiperidin-1-yl)benzoic acids. [M+H]⁺ calcd for C₃₅H₃₂N₆O₃: 585.25. Found: 585.15.

### Example 828

### (±)-N-(1-(4-(Cyclopropanecarboxamido)phenyl)-1H-indazol-5-yl)-4-(3-hydroxymethylpiperidin-1-yl)benzamide (Compound 928)

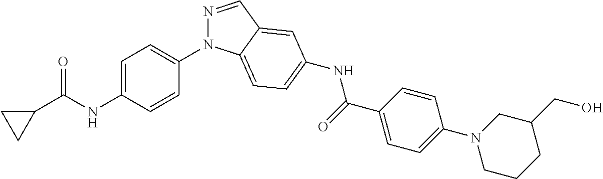 [SMILES: O=C(Nc1ccc2c(cnn2-c2ccc(NC(=O)C3CC3)cc2)c1)c1ccc(N2CCCC(CO)C2)cc1]

Compound 928 was prepared according to the procedure described in Scheme IV from 5-amino-1-(4-aminophenyl)indazole, cyclopropanecarboxylic, and 4-(3-hydroxymethylpiperidin-1-yl)benzoic acids. [M+H]⁺ calcd for C₃₀H₃₁N₅O₃: 510.24. Found: 510.12.

### Example 829

### N-(1-(4-(Cyclopropanecarboxamido)phenyl)-1H-indazol-5-yl)-4-(4-oxopiperidin-1-yl)benzamide (Compound 929)

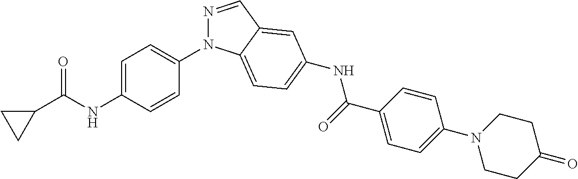 [SMILES: O=C1CCN(c2ccc(C(=O)Nc3ccc4c(cnn4-c4ccc(NC(=O)C5CC5)cc4)c3)cc2)CC1]

Compound 929 was prepared according to the procedure described in Scheme IV from 5-amino-1-(4-aminophenyl)indazole, cyclopropanecarboxylic, and 4-(4-oxopiperidin-1-yl)benzoic acids. [M+H]⁺ calcd for C₂₉H₂₇N₅O₃: 494.21. Found: 494.06.

### Example 830

### N-(1-(4-(Cyclopropanecarboxamido)phenyl)-1H-indazol-5-yl)-1-methyl-1H-indole-5-carboxamide (Compound 930)

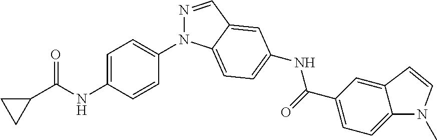 [SMILES: Cn1ccc2cc(C(=O)Nc3ccc4c(cnn4-c4ccc(NC(=O)C5CC5)cc4)c3)ccc21]

Compound 930 was prepared according to the procedure described in Scheme IV from 5-amino-1-(4-aminophenyl)indazole, cyclopropanecarboxylic, and 1-methyl-1H-indole-5-carboxylic acids. [M+H]⁺ calcd for C₂₇H₂₃N₅O₂: 450.19. Found: 450.06.

### Example 831

### N-(1-(4-(Cyclopropanecarboxamido)phenyl)-1H-indazol-5-yl)-4-(4-(hydroxyimino)piperidin-1-yl)benzamide (Compound 931)

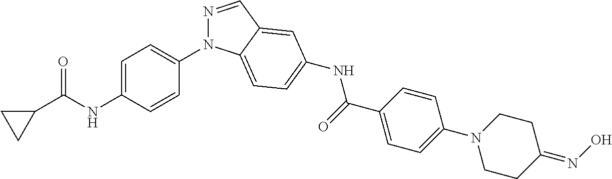 [SMILES: O=C(Nc1ccc2c(cnn2-c2ccc(NC(=O)C3CC3)cc2)c1)c1ccc(N2CCC(=NO)CC2)cc1]

Compound 931 was prepared according to the procedure described in Scheme IV from 5-amino-1-(4-aminophenyl)indazole, cyclopropanecarboxylic, and 4-(4-(hydroxyimino)piperidin-1-yl)benzoic acids. [M+H]⁺ calcd for C₂₉H₂₈N₆O₃: 509.22. Found: 509.10.

### Example 832

### (±)-N-(1-(4-(2-Methylcyclopropanecarboxamido)phenyl)-1H-indazol-5-yl)-4-(4-hydroxypiperidin-1-yl)benzamide (Compound 932)

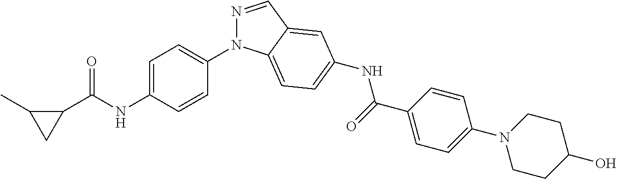 [SMILES: CC1CC1C(=O)Nc1ccc(-n2ncc3cc(NC(=O)c4ccc(N5CCC(O)CC5)cc4)ccc32)cc1]

Compound 932 was prepared according to the procedure described in Scheme IV from 5-amino-(4-aminophenyl)indazole, 2-methylcyclopropanecarboxylic, and 4-(4-hydroxypiperidin-1-yl)benzoic acids. [M+H]⁺ calcd for C₃₀H₃₁N₅O₃: 510.24. Found: 510.13.

### Example 833

### N-(1-(4-(Cyclopropanecarboxamido)phenyl)-1H-indazol-5-yl)-4-(4-hydroxy-4-(pyrrolidin-1-ylmethyl)piperidin-1-yl)benzamide (Compound 933)

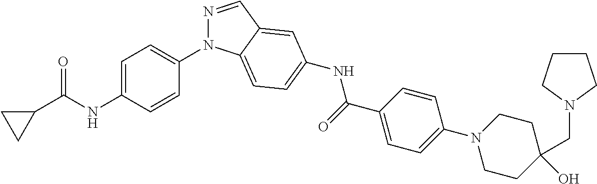 [SMILES: O=C(Nc1ccc2c(cnn2-c2ccc(NC(=O)C3CC3)cc2)c1)c1ccc(N2CCC(O)(CN3CCCC3)CC2)cc1]

Compound 933 was prepared according to the procedure described in Scheme IV from 5-amino-(4-aminophenyl)indazole, cyclopropanecarboxylic, and 4-(4-hydroxy-4-(pyrrolidin-1-ylmethyl)piperidin-1-yl)benzoic acids.

### Example 834

### N-(1-(4-(Cyclopropanecarboxamido)phenyl)-1H-indazol-5-yl)-4-(2-hydroxyethylamino)benzamide (Compound 934)

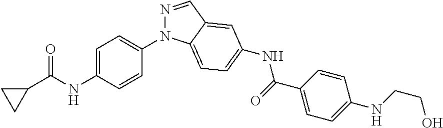 [SMILES: O=C(Nc1ccc2c(cnn2-c2ccc(NC(=O)C3CC3)cc2)c1)c1ccc(NCCO)cc1]

Compound 934 was prepared according to the procedure described in Scheme IV from 5-amino-(4-aminophenyl)indazole, cyclopropanecarboxylic, and 4-(2-hydroxyethylamino)benzoic acids. [M+H]⁺ calcd for C₂₆H₂₅N₅O₃: 456.20. Found 456.03.

### Example 835

### (±)-N-(1-(4-(Cyclopropanecarboxamido)phenyl)-1H-indazol-5-yl)-4-((2,3-dihydroxypropyl)amino)benzamide (Compound 935)

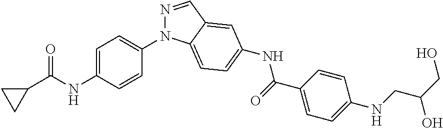 [SMILES: O=C(Nc1ccc2c(cnn2-c2ccc(NC(=O)C3CC3)cc2)c1)c1ccc(NCC(O)CO)cc1]

Compound 935 was prepared according to the procedure described in Scheme IV from 5-amino-1-(4-aminophenyl)indazole, cyclopropanecarboxylic, and 4-(2,3-dihydroxypropylamino)benzoic acids. [M+H]⁺ calcd for C₂₇H₂₇N₅O₄: 486.21. Found 486.03.

### Example 836

### N-(1-(4-(Cyclopropanecarboxamido)phenyl)-1H-indazol-5-yl)-4-(4-methoxypiperidin-1-yl)benzamide (Compound 936)

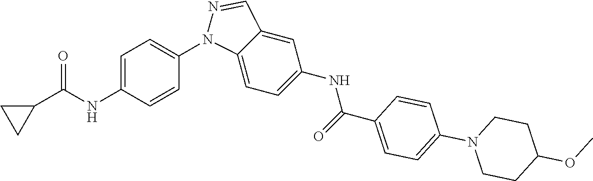 [SMILES: COC1CCN(c2ccc(C(=O)Nc3ccc4c(cnn4-c4ccc(NC(=O)C5CC5)cc4)c3)cc2)CC1]

Compound 936 was prepared according to the procedure described in Scheme IV from 5-amino-1-(4-aminophenyl)indazole, cyclopropanecarboxylic, and 4-(4-methoxypiperidin-1-yl)benzoic acids. [M+H]⁺ calcd for C₃₀H₃₁N₅O₃: 510.24. Found: 510.12.

### Example 837

### N-(1-(4-(Cyclopropanecarboxamido)phenyl)-1H-indazol-5-yl)-4-(4-(3-hydroxypropyl)piperazin-1-yl)benzamide (Compound 937)

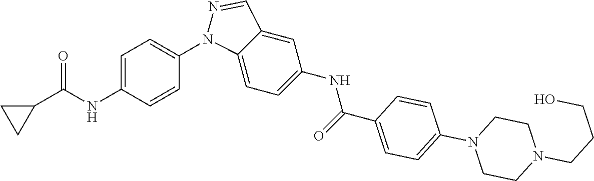 [SMILES: O=C(Nc1ccc2c(cnn2-c2ccc(NC(=O)C3CC3)cc2)c1)c1ccc(N2CCN(CCCO)CC2)cc1]

Compound 937 was prepared according to the procedure described in Scheme IV from 5-amino-1-(4-aminophenyl)indazole, cyclopropanecarboxylic, and 4-(4-(3-hydroxypropyl)piperazin-1-yl)benzoic acids. [M+H]⁺ calcd for C₃₁H₃₄N₆O₃: 539.17. Found: 539.16.

### Example 838

### (±)-N-(1-(4-(Cyclopropanecarboxamido)phenyl)-1H-indazol-5-yl)-4-(3-hydroxypyrrolidin-1-yl)benzamide (Compound 938)

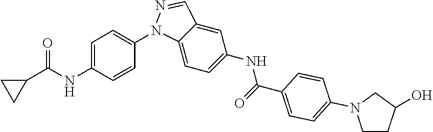 [SMILES: O=C(Nc1ccc2c(cnn2-c2ccc(NC(=O)C3CC3)cc2)c1)c1ccc(N2CCC(O)C2)cc1]

Compound 938 was prepared according to the procedure described in Scheme IV from 5-amino-(4-aminophenyl)indazole, cyclopropanecarboxylic, and 4-(3-hydroxypyrrolidin-1-yl)benzoic acids. ¹H NMR (500 MHz, CD₃OD) δ 8.19 (s, 1H), 8.17 (s, 1H), 7.86 (d, J=8.5 Hz, 2H), 7.77 (d, J=8.5 Hz, 2H), 7.70-7.63 (m, 4H), 6.62 (d, J=8.5 Hz, 2H), 4.55 (s, 1H), 3.56-3.50 (m, 2H), 3.43 (dd, J=3, 9 Hz, 1H), 2.20-2.13 (m, 1H), 2.08-2.04 (m, 1H), 1.83-1.78 (m, 1H), 1.28 (s, 1H), 1.00-0.97 (m, 2H), 0.90-0.86 (m, 2H).

### Example 839

### N-(1-(4-(Cyclopropanecarboxamido)phenyl)-1H-indazol-5-yl)-1-(2-hydroxyethyl)-1H-indole-5-carboxamide (Compound 939)

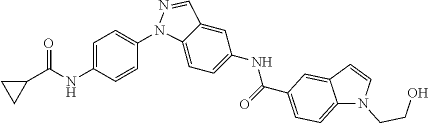 [SMILES: O=C(Nc1ccc2c(cnn2-c2ccc(NC(=O)C3CC3)cc2)c1)c1ccc2c(ccn2CCO)c1]

Compound 939 was prepared according to the procedure described in Scheme IV from 5-amino-1-(4-aminophenyl)indazole, cyclopropanecarboxylic, and 1-(2-hydroxyethyl)-1H-indole-5-carboxylic acids. [M+H]⁺ calcd for C₂₈H₂₅N₅O₃: 480.20. Found 480.03.

### Example 840

### N-(1-(4-(Cyclopropanecarboxamido)phenyl)-1H-indazol-5-yl)-4-(4-hydroxymethylpiperidin-1-yl)benzamide (Compound 940)

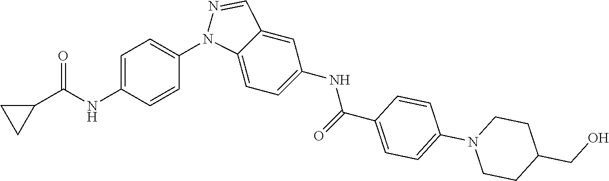 [SMILES: O=C(Nc1ccc2c(cnn2-c2ccc(NC(=O)C3CC3)cc2)c1)c1ccc(N2CCC(CO)CC2)cc1]

Compound 940 was prepared according to the procedure described in Scheme IV from 5-amino-1-(4-aminophenyl)indazole, cyclopropanecarboxylic, and 4-(4-hydroxymethylpiperidin-1-yl)benzoic acids. ¹H NMR (500 MHz, DMSO-d₆) δ 10.38 (s, 1H), 10.04 (s, 1H), 8.36 (s, 1H), 8.31 (s, 1H), 7.88 (d, J=9 Hz, 2H), 7.81-7.75 (m, 6H), 7.69 (d, J=8.5 Hz, 2H), 7.01 (d, J=9.5 Hz, 2H), 4.72 (d, J=4 Hz, 2H), 1.85-1.79 (m, 2H), 1.46-1.42 (m, 2H), 0.86-0.81 (m, 4H).

### Example 841

### N-(1-(4-(Cyclopropanecarboxamido)phenyl)-1H-indazol-5-yl)-1-(3-hydroxypropyl)-1H-indole-6-carboxamide (Compound 941)

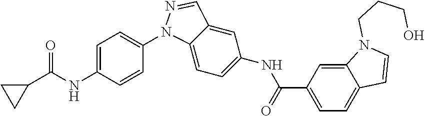 [SMILES: O=C(Nc1ccc2c(cnn2-c2ccc(NC(=O)C3CC3)cc2)c1)c1ccc2ccn(CCCO)c2c1]

Compound 941 was prepared according to the procedure described in Scheme IV from 5-amino-(4-aminophenyl)indazole, cyclopropanecarboxylic, and 1-(3-hydroxypropyl)-1H-indole-6-carboxylic acids. [M+H]⁺ calcd for C₂₉H₂₇N₅O₃: 494.21. Found: 494.00.

### Example 842

### N-(1-(4-(Cyclopropanecarboxamido)phenyl)-1H-indazol-5-yl)-1H-indole-5-carboxamide (Compound 942)

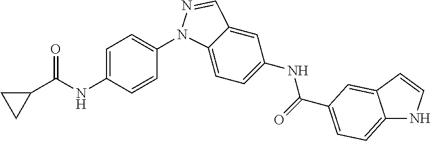 [SMILES: O=C(Nc1ccc2c(cnn2-c2ccc(NC(=O)C3CC3)cc2)c1)c1ccc2[nH]ccc2c1]

Compound 942 was prepared according to the procedure described in Scheme IV from 5-amino-1-(4-aminophenyl)indazole, cyclopropanecarboxylic, and 1H-indole-5-carboxylic acids. [M+H]⁺ calcd for C₂₆H₂₁N₅O₂: 436.17. Found: 436.02.

### Example 843

### (±)-4-(4-Hydroxypiperidin-1-yl)-N-(1-(4-((trans)-2-phenylcyclopropanecarboxamido)phenyl)-1H-indazol-5-yl)benzamide (Compound 943)

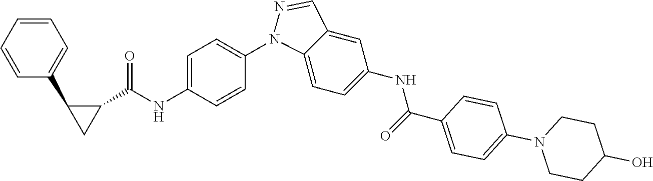 [SMILES: O=C(Nc1ccc2c(cnn2-c2ccc(NC(=O)[C@@H]3C[C@H]3c3ccccc3)cc2)c1)c1ccc(N2CCC(O)CC2)cc1]

Compound 943 was prepared according to the procedure described in Scheme IV from 5-amino-1-(4-aminophenyl)indazole, 2-trans-phenylcyclopropanecarboxylic, and 4-(4-hydroxypiperidin-1-yl)benzoic acids. [M+H]⁺ calcd for C₃₅H₃₃N₅O₃: 572.26. Found 572.17.

### Example 844

### N-(3-(4-(Cyclopropanecarboxamido)phenyl)-1H-indol-6-yl)-1-methyl-1H-indole-5-carboxamide (Compound 944)

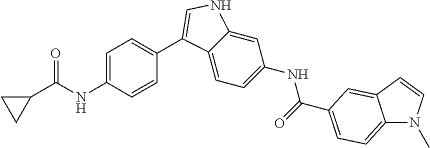 [SMILES: Cn1ccc2cc(C(=O)Nc3ccc4c(-c5ccc(NC(=O)C6CC6)cc5)c[nH]c4c3)ccc21]

Compound 944 was prepared according to the general procedure described in Scheme IV. Preparation of 3-bromo-6-nitro-1H-indole: To a solution of 6-nitro-1H-indole (3.00 g, 18.5 mmol) in methylene chloride (100 mL) was added N-bromosuccinimide (3.29 g, 18.5 mmol) as four equal portions over 2 h. The reaction was stirred overnight, and the formed precipitate was filtered off and rinsed with water. The collected, biphasic filtrate was separated by removing the organic phase and extracting the aqueous phase with methylene chloride (3×200 mL). The combined organic phases were concentrated to give a crude solid. The solids collected from the filtration and the extractions were recrystallized using methanol/water and gave 3-bromo-6-nitro-1H-indole (3.95 g, 16.4 mmol, 89%) as a yellow solid after filtration.

Preparation of 3-bromo-6-nitro-1-(phenylsulfonyl)-1H-indole: Sodium hydride (461 mg, 11.5 mmol) was slowly added to a solution of 3-bromo-6-nitro-1H-indole (2.32 g, 9.61 mmol) in tetrahydrofuran (10.0 mL). After stirring for 5 min, benzenesulfonyl chloride (1.47 mL, 11.5 mmol) was added dropwise over 10 min. The reaction was stirred an additional 1.5 h before quenching with 5.0 mL of methanol, followed by 10.0 mL water and 20.0 mL methylene chloride. The precipitate was filtered off and washed with an excess of methylene chloride and methanol to give a crime-colored powder for 3-bromo-6-nitro-1-(phenylsulfonyl)-1H-indole (2.80 g, 7.35 mmol, 76%).

Preparation of 3-bromo-1-(phenylsulfonyl)-1H-indol-6-amine: To a slurry of 3-bromo-6-nitro-1-(phenylsulfonyl)-1H-indole (2.80 g, 7.35 mmol) in ethanol (73.5 mL) was added a solution of tin (II) chloride monohydrate (6.63 g, 29.4 mmol) in water (11.0 mL) at 30° C. Once the addition was complete, the reaction was heated to reflux for 3.5 h, then quenched with 2.0 N sodium hydroxide to a pH of 8. The remaining slurry was diluted with ethyl acetate (100 mL), and the aqueous layer was removed and filtered. The organic phase was washed with 2.0 N sodium hydroxide (2×100 mL), and the combined aqueous layers were extracted with ethyl acetate (3×200 mL). The combined organic phases were concentrated to give a tacky, orange solid for 3-bromo-1-(phenylsulfonyl)-1H-indol-6-amine (2.35 g, 6.69 mmol, 91%).

Preparation of 3-(4-nitrophenyl)-1H-indol-6-amine: A 1.0 M aqueous potassium carbonate solution (9.96 mL, 9.96 mmol) was added to a mixture of 3-bromo-1-(phenylsulfonyl)-1H-indol-6-amine (1.00 g, 2.84 mmol), (4-nitrophenyl)boronic acid (1.19 g, 7.12 mmol) and palladium tetrakis(triphenylphosphine) (164 mg, 0.14 mmol) in tetrahydrofuran (13.7 mL). The biphasic mixture was heated in a microwave at 130° C. for 10 min and was then diluted with a saturated solution of sodium bicarbonate (10.0 mL) and ethyl acetate (10.0 mL). The organic layer was washed with a saturated solution of sodium bicarbonate (2×10.0 mL), and the combined aqueous layers were extracted with ethyl acetate (3×30.0 mL). The combined organic phases were dried over magnesium sulfate, filtered and concentrated. The crude residue was purified by flash chromatography (10-25-50-71% ethyl acetate/hexane) to give an orange solid for 3-(4-nitrophenyl)-1-(phenylsulfonyl)-1H-indol-6-amine (726 mg, 1.85 mmol, 65%).

3-(4-nitrophenyl)-1H-indol-6-amine. To a solution of the protected indol-6-amine (300 mg, 0.76 mmol) in methanol (9.00 mL) was added potassium hydroxide (941 mg, 16.8 mmol). The reaction was warmed to 70° C. for 4 h. After that time, the reaction was diluted with water (10.0 mL) and methylene chloride (15.0 mL) and the organic phase was removed. The aqueous layer was extracted with methylene chloride (3×15.0 mL), and the combined organic phases were concentrated. The crude residue that remained was purified by flash chromatography (10-25-50% ethyl acetate/hexane) to give a burgundy red solid for 3-(4-nitrophenyl)-1H-indol-6-amine (66.3 mg, 0.26 mmol, 34%).

Preparation of 1-methyl-N-(3-(4-nitrophenyl)-1H-indol-6-yl)-1H-indole-5-carboxamide: A mixture of 3-(4-nitrophenyl)-1H-indol-6-amine (66.3 mg, 0.26 mmol), 1-methyl-1H-indole-5-carboxylic acid (50.4 mg, 0.29 mmol), hydroxybenzotriazole (3.5 mg, 0.03 mmol) and triethylamine (40.1 μL, 0.29 mmol) in DMF (1.00 mL) was stirred for 5 min before 1-ethyl-3-(3-dimethylaminopropyl) carbodiimide hydrochloride (55.2 mg, 0.29 mmol) was added. The reaction was stirred overnight at room temperature and was then diluted with water (5.0 mL). The precipitate that formed was filtered off and sequentially washed with excess methylene chloride and methanol. The solid was recrystallized using hot methanol and gave a bright yellow solid for 1-methyl-N-(3-(4-nitrophenyl)-1H-indol-6-yl)-1H-indole-5-carboxamide (59.8 mg, 0.15 mmol, 56%) after filtration.

Preparation of N-(3-(4-aminophenyl)-1H-indol-6-yl)-1-methyl-1H-indole-5-carboxamide: To a slurry of 1-methyl-N-(3-(4-nitrophenyl)-1H-indol-6-yl)-1H-indole-5-carboxamide (59.0 mg, 0.14 mmol) in ethanol (1.50 mL) under an atmosphere of nitrogen was added 20 wt % palladium hydroxide on carbon (5.9 mg, 10 wt %), followed by a balloon of hydrogen. The reaction was stirred overnight at room temperature and was then filtered through a pad of Celite, rinsing with excess methanol and methylene chloride. The filtrate was concentrated to give a grey-pink solid for N-(3-(4-aminophenyl)-1H-indol-6-yl)-1-methyl-1H-indole-5-carboxamide (56.9 mg, 0.14 mmol, 100%).

Preparation of Compound 944: A mixture of N-(3-(4-aminophenyl)-1H-indol-6-yl)-1-methyl-1H-indole-5-carboxamide (55.8 mg, 0.15 mmol), cyclopropanecarboxylic acid (12.8 μL, 0.16 mmol), hydroxybenzotriazole (1.9 mg, 0.02 mmol) and triethylamine (22.5 μL, 0.16 mmol) in N,N-dimethylformamide (1.00 mL) was stirred for 5 min before 1-ethyl-3-(3-dimethylaminopropyl)carbodiimide hydrochloride (30.9 mg, 0.16 mmol) was added. The reaction was stirred overnight at room temperature and was then diluted with water (5.0 mL).

The precipitate that formed was filtered off and sequentially washed with excess methylene chloride and methanol. The solid was recrystallized using hot methanol and gave a yellow solid for N-(3-(4-(cyclopropanecarboxamido)phenyl)-1H-indol-6-yl)-1-methyl-1H-indole-5-carboxamide (46.8 mg, 0.10 mmol, 70%) after filtration. MS [M+H]⁺ calcd for C₂₈H₂₄N₄O₂: 449.04. Found: 449.05.

### Example 845

### 1-(4-(Cyclopropanecarboxamido)phenyl)-N-(4-(4-hydroxypiperidin-1-yl)phenyl)-1H-indazole-5-carboxamide (Compound 945)

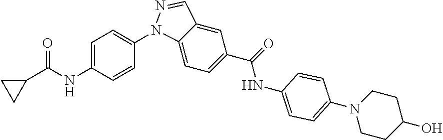 [SMILES: O=C(Nc1ccc(N2CCC(O)CC2)cc1)c1ccc2c(cnn2-c2ccc(NC(=O)C3CC3)cc2)c1]

Compound 945 was prepared according to the general procedure described in Scheme IV. Preparation of N-(4-(4-hydroxypiperidin-1-yl)phenyl)-1H-indazole-5-carboxamide: 1H-indazole-5-carboxylic acid hydrochloride (100 mg, 0.50 mmol), hydroxybenzotriazole (7 mg, 0.05 mmol), triethylamine (0.2 mL, 1.3 mmol), and 1-(4-aminophenyl)piperidin-4-ol (193 mg, 1.00 mmol) were taken up in DMF (2.51 mL) and stirred. EDC (106 mg, 0.05 mmol) was added to the solution last. After the addition, the solution was stirred at room temperature for 24 h. The solution was partitioned between water and EtOAc. The aqueous layer was extracted with EtOAc. The combined organic layers were washed with brine and dried over Na₂SO₄. Filtration and concentration gave the crude amide. Purification via flash chromatography 0-10% MeOH/CH₂Cl₂ gave 55 mg (32%) of the product as a white solid.

Preparation of 1-(4-aminophenyl)-N-(4-(4-hydroxypiperidin-1-yl)phenyl)-1H-indazole-5-carboxamide: N-(4-(4-Hydroxypiperidin-1-yl)phenyl)-1H-indazole-5-carboxamide (10 mg, 0.03 mmol), 4-fluoronitrobenze (3.1 uL, 0.03 mmol) and potassium carbonate (4 mg, 0.03 mmol) were taken up in DMSO (0.3 mL). The solution was heated to 120° C. and stirred for 24 h. After the solution was cooled it was diluted with water until a precipitate formed and stirred well for 5 min. Filtration gave a yellow solid, which was then washed well with water, followed by hexanes. After the solid was dried under vacuum it was taken up in methanol and stirred at RT under nitrogen. The solution was treated with Pd(OH)₂ (3 mg) and placed under a balloon of H₂ gas. After stirring at RT for 24 h, the catalyst was removed via filtration through celite. The filtrate was concentrated under reduced pressure to give 10.23 mg (80%) of a crude product.

Preparation of Compound 945: 1-(4-Aminophenyl)-N-(4-(4-hydroxypiperidin-1-yl)phenyl)-1H-indazole-5-carboxamide (10 mg, 0.02 mmol), EDC (6.7 mg, 0.03 mmol) and cyclopropanecarboxylic acid (2.1 mg, 0.02 mmol) were taken up in pyridine (0.23 mL). The solution was heated to 60° C. and stirred for 1 h. The solution was partitioned between CH₂Cl₂ and water. The aqueous layer was extracted with CH₂Cl₂. The combined organic layers were washed with brine and dried over Na₂SO₄. Filtration and concentration gave the crude cyclopropyl amide. Purification via flash chromatography 0-10% MeOH/CH₂Cl₂ gave 8.8 mg (76%) of Compound 945. [M+H]⁺ calcd for C₂₉H₂₉N₅O₃: 496.23. Found: 496.02.

### Example 846

### 4-(5-(cyclopropanecarboxamido)-1H-indol-1-yl)-N-(4-(4-hydroxypiperidin-1-yl)phenyl)benzamide (Compound 946)

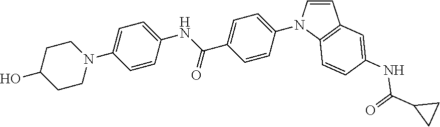 [SMILES: O=C(Nc1ccc(N2CCC(O)CC2)cc1)c1ccc(-n2ccc3cc(NC(=O)C4CC4)ccc32)cc1]

Compound 946 was prepared according to the procedure described in Scheme IV from 1-(4-aminophenyl)-1H-indole-5-carboxylic acid, cyclopropanecarboxylic acid, and 4-(4-hydroxypiperidin-1-yl)aniline. [M+H]⁺ calcd for C₃₀H₃₀N₄O₃: 495.23. Found: 495.08.

### Example 847

### N-(1-(4-(Cyclopropylcarbamoyl)phenyl)-1H-indol-5-yl)-1-(2-hydroxyethyl)-1H-indole-5-carboxamide (Compound 947)

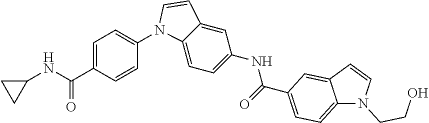 [SMILES: O=C(Nc1ccc2c(ccn2-c2ccc(C(=O)NC3CC3)cc2)c1)c1ccc2c(ccn2CCO)c1]

Compound 947 was prepared according to the procedure described in Scheme IV from 4-(5-amino-1H-indol-1-yl)benzoic acid, cyclopropaneamine, and 1-(2-hydroxy ethyl)-1H-indole-5-carboxylic acid. [M+H]⁺ calcd for C₂₉H₂₆N₄O₃: 479.20. Found: 479.01.

### Example 848

### (±)-4-(4-Hydroxypiperidin-1-yl)-N-(1-(4-(2-hydroxymethylcyclopropanecarboxamido)phenyl)-1H-indazol-5-yl)benzamide (Compound 948)

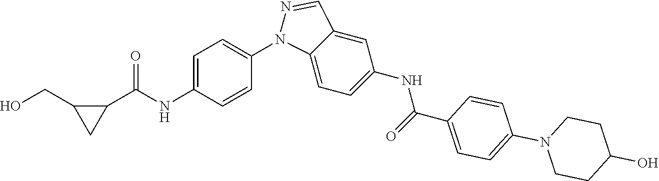 [SMILES: O=C(Nc1ccc2c(cnn2-c2ccc(NC(=O)C3CC3CO)cc2)c1)c1ccc(N2CCC(O)CC2)cc1]

Compound 948 was prepared according to the procedure described in Scheme IV from 5-amino-(4-aminophenyl)indazole, 2-hydroxymethylcyclopropanecarboxylic, and 4-(4-hydroxypiperidin-1-yl)benzoic acids. ¹H NMR (500 MHz, CD₃OD) δ 8.21 (m, 2H), 7.88 (d, J=9.1 Hz, 2H), 7.79 (d, J=8.8 Hz, 2H), 7.74-7.65 (m, 4), 7.04 (d, J=8.8 Hz, 2H), 3.84-3.77 (m, 3H), 3.09-3.04 (m, 2H), 1.98-1.96 (m, 1H), 1.65-1.58 (m, 4H), 1.22-1.19 (m, 1H), 0.94-0.88 (m, 2H).

### Example 849

### N-Cyclopropyl-1-(4-(4-(4-hydroxypiperidin-1-yl)benzamido)phenyl)-1H-indole-5-carboxamide (Compound 949)

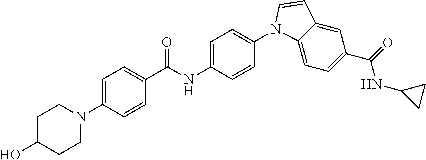 [SMILES: O=C(Nc1ccc(-n2ccc3cc(C(=O)NC4CC4)ccc32)cc1)c1ccc(N2CCC(O)CC2)cc1]

Compound 949 was prepared according to the procedure described in Scheme IV from cylopropylamine, 1-(4-aminophenyl)-1H-indole-5-carboxylic, and 4-(4-hydroxypiperidin-1-yl)benzoic acids. [M+H]⁺ calcd for C₃₀H₃₁N₄O₃: 495.24. Found: 495.08.

### Example 850

### N-Cyclopropyl-1-(4-((4-(4-hydroxypiperidin-1-yl)phenyl)carbamoyl)phenyl)-1H-indazole-5-carboxamide (Compound 950)

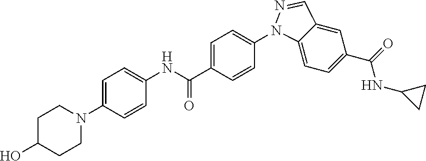 [SMILES: O=C(Nc1ccc(N2CCC(O)CC2)cc1)c1ccc(-n2ncc3cc(C(=O)NC4CC4)ccc32)cc1]

Compound 950 was prepared according to the procedure described in Scheme IV from cylopropylamine, 1-(4-carboxyphenyl)-1H-indazole-5-carboxylic acid, and 4-(4-hydroxypiperidin-1-yl)aniline. [M+H]⁺ calcd for C₂₉H₂₉N₅O₃: 496.23. Found: 496.02.

### Example 851

### 4-(4-(Allyloxy)piperidin-1-yl)-N-(1-(4-(cyclopropylcarbamoyl)phenyl)-1H-indol-5-yl)benzamide (Compound 951)

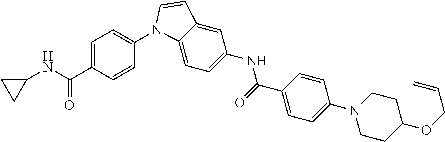 [SMILES: C=CCOC1CCN(c2ccc(C(=O)Nc3ccc4c(ccn4-c4ccc(C(=O)NC5CC5)cc4)c3)cc2)CC1]

Compound 951 was prepared according to the procedure described in Scheme IV from 4-(5-amino-1H-indol-1-yl)benzoic acid, cyclopropaneamine, and 4-(4-allyloxypiperidin-1-yl)benzoic acid. [M+H]⁺ calcd for C₃₃H₃₄N₄O₃: 535.26. Found 535.10.

### Example 852

### N-Cyclopropyl-4-(5-(4-((2-hydroxyethyl)(methyl)amino)benzamido)-1H-indol-1-yl)benzamide (Compound 952)

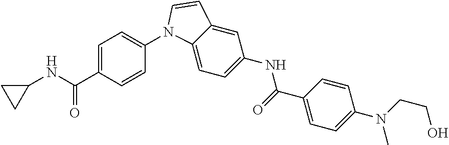 [SMILES: CN(CCO)c1ccc(C(=O)Nc2ccc3c(ccn3-c3ccc(C(=O)NC4CC4)cc3)c2)cc1]

Compound 952 was prepared according to the procedure described in Scheme IV from 4-(5-amino-1H-indol-1-yl)benzoic acid, cyclopropaneamine, and 4-(N-2-hydroxyethyl-N-methylamino)benzoic acid. [M+H]⁺ calcd for C₂₈H₂₈N₄O₃: 469.22. Found 468.96.

### Example 853

### 4-(4-Hydroxypiperidin-1-yl)-N-(1-(4-(thiazol-2-ylcarbamoyl)phenyl)-1H-indol-5-yl)benzamide (Compound 953)

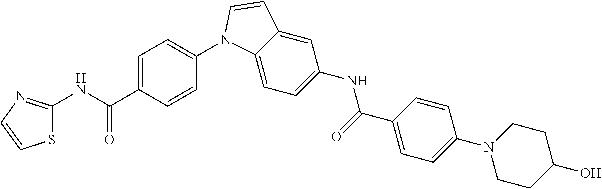 [SMILES: O=C(Nc1ccc2c(ccn2-c2ccc(C(=O)Nc3nccs3)cc2)c1)c1ccc(N2CCC(O)CC2)cc1]

Compound 953 was prepared according to the procedure described in Scheme IV from 4-(5-amino-1H-indol-1-yl)benzoic acid, 2-aminothiazole, and 4-(4-hydroxypiperidin-1-yl)benzoic acid. [M+H]⁺ calcd for C₃₀H₂₇N₅O₃S: 538.18. Found: 539.02.

### Example 854

### 4-(4-Hydroxypiperidin-1-yl)-N-(1-(4-(4-methylthiazol-2-ylcarbamoyl)phenyl)-1H-indol-5-yl)benzamide (Compound 954)

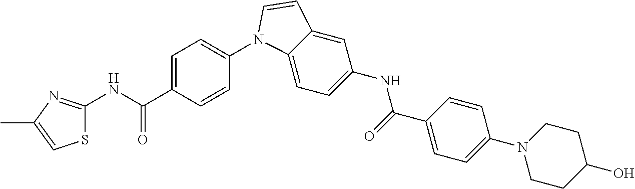 [SMILES: Cc1csc(NC(=O)c2ccc(-n3ccc4cc(NC(=O)c5ccc(N6CCC(O)CC6)cc5)ccc43)cc2)n1]

Compound 954 was prepared according to the procedure described in Scheme IV from 4-(5-amino-1H-indol-1-yl)benzoic acid, 2-amino-4-methylthiazole, and 4-(4-hydroxypiperidin-1-yl)benzoic acid. [M+H]⁺ calcd for C₃₁H₂₉N₅O₃S: 552.10. Found: 552.05.

### Example 855

### 4-(4-Hydroxypiperidin-1-yl)-N-(1-(4-(2-methyl-1H-indole-5-ylcarbamoyl)phenyl)-1H-indol-5-yl)benzamide (Compound 955)

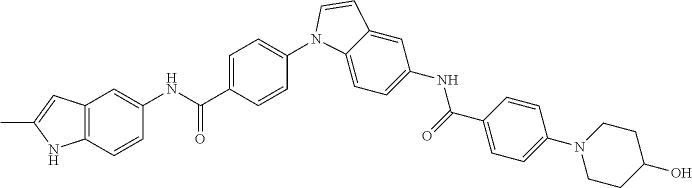 [SMILES: Cc1cc2cc(NC(=O)c3ccc(-n4ccc5cc(NC(=O)c6ccc(N7CCC(O)CC7)cc6)ccc54)cc3)ccc2[nH]1]

Compound 955 was prepared according to the procedure described in Scheme IV from 5-amino-2-methylindole, 4-(5-amino-1H-indol-1-yl)benzoic, and 4-(4-hydroxypiperidin-1-yl)benzoic acids. ¹H NMR (500 MHz, DMSO-d₆) δ 10.85 (s, 1H), 10.11 (s, 1H), 9.90 (s, 1H), 8.17 (d, J=8.5 Hz, 2H), 8.14 (d, J=2 Hz, 1H), 7.88 (d, J=9 Hz, 2H), 7.86 (d, J=2 Hz, 1H), 7.77 (d, J=8.5 Hz, 2H), 7.75 (d, J=3 Hz, 1H), 7.65 (d, J=9 Hz, 1H), 7.56 (dd, J=2, 9 Hz, 1H), 7.33 (dd, J=1.5, 8.5 Hz, 1H), 7.23 (d, J=8.5 Hz, 1H), 7.00 (d, J=9 Hz, 2H), 6.74 (d, J=3.5 Hz, 1H), 6.11 (t, J=1 Hz, 1H), 4.71 (d, J=4 Hz, 1H), 3.72-3.66 (m, 3H), 3.00 (dd, J=3, 10 Hz, 2H), 2.37 (s, 3H), 1.83-1.80 (m, 2H), 1.47-1.40 (m, 2H).

### Example 856

### 4-(4-Hydroxypiperidin-1-yl)-N-(1-(4-((1-(2-morpholinoethyl)-1H-indol-5-yl)carbamoyl)phenyl)-1H-indol-5-yl)benzamide (Compound 956)

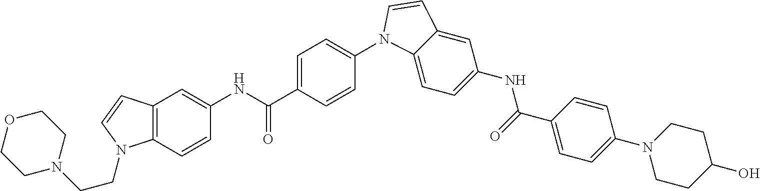 [SMILES: O=C(Nc1ccc2c(ccn2CCN2CCOCC2)c1)c1ccc(-n2ccc3cc(NC(=O)c4ccc(N5CCC(O)CC5)cc4)ccc32)cc1]

Compound 956 was prepared according to the procedure described in Scheme IV from 1-(2-morpholinoethyl)-5-amino-1H-indole, 4-(5-amino-1H-indol-1-yl)benzoic, and 4-(4-hydroxypiperidin-1-yl)benzoic acids. [M+H]⁺ calcd for C₄₁H₄₂N₆O₄: 683.33. Found: 683.31.

### Example 857

### N-(5-Fluoropyridin-2-yl)-4-(5-(4-(4-hydroxypiperidin-1-yl)benzamido)-1H-indol-1-yl)benzamide (Compound 957)

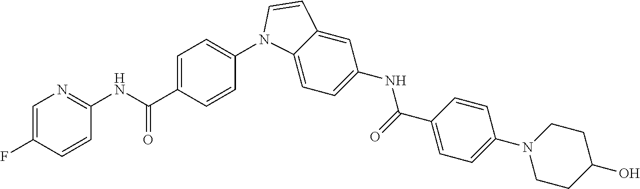 [SMILES: O=C(Nc1ccc2c(ccn2-c2ccc(C(=O)Nc3ccc(F)cn3)cc2)c1)c1ccc(N2CCC(O)CC2)cc1]

Compound 957 was prepared according to the procedure described in Scheme IV from 2-amino-5-fluoropyridine, 4-(5-amino-1H-indol-1-yl)benzoic, and 4-(4-hydroxypiperidin-1-yl)benzoic acids. ¹H NMR (500 MHz, DMSO-d₆) δ 11.01 (s, 1H), 9.90 (s, 1H), 8.42 (d, J=3.5 Hz, 1H), 8.27 (d, J=4 Hz, 1H), 8.24 (dd, J=1.5, 6.5 Hz, 2H), 8.14 (d, J=2 Hz, 1H), 7.88 (d, J=9 Hz, 2H), 7.84-7.80 (m, 1H), 7.78-7.77 (m, 1H), 7.76 (t, J=3.5 Hz, 2H), 7.66 (d, J=9 Hz, 1H), 7.56 (dd, J=2, 9 Hz, 1H), 7.00 (d, J=9 Hz, 2H), 6.75 (d, J=3.5 Hz, 1H), 4.70 (d, J=4 Hz, 1H), 3.71-3.66 (m, 3H), 3.03-2.98 (m, 1H), 1.83-1.79 (m, 2H), 1.47-1.40 (m, 2H).

### Example 858

### 4-(4-Hydroxypiperidin-1-yl)-N-(1-(4-((1H-indol-6-yl)carbamoyl)phenyl)-1H-indol-5-yl)benzamide (Compound 958)

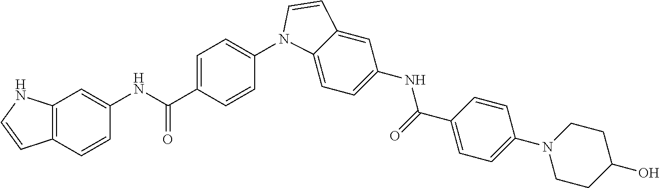 [SMILES: O=C(Nc1ccc2c(ccn2-c2ccc(C(=O)Nc3ccc4cc[nH]c4c3)cc2)c1)c1ccc(N2CCC(O)CC2)cc1]

Compound 958 was prepared according to the procedure described in Scheme IV from 6-aminoindole, 4-(5-amino-1H-indol-1-yl)benzoic, and 4-(4-hydroxypiperidin-1-yl)benzoic acids. ¹H NMR (500 MHz, DMSO-d₆) δ 11.05 (s, 1H), 10.23 (s, 1H), 9.90 (s, 1H), 8.18 (d, J=8.5 Hz, 2H), 8.13 (d, J=2 Hz, 1H), 8.10 (s, 1H), 7.87 (d, J=10 Hz, 2H), 7.77 (d, J=9 Hz, 2H), 7.74 (d, J=3.5 Hz, 1H), 7.65 (d, J=9 Hz, 1H), 7.56 (dd, J=2, 9.5 Hz, 1H), 7.48 (d, J=8.5 Hz, 1H), 7.29 (m, 2H), 6.99 (d, J=9 Hz, 2H), 6.74 (d, J=3 Hz, 1H), 6.38 (m, 1H), 4.70 (d, J=4.5 Hz, 1H), 3.69 (m, 3H), 1.80 (m, 2H), 1.44 (m, 2H).

### Example 859

### 4-(4-Hydroxypiperidin-1-yl)-N-(1-(4-(cyclopropylcarbamoyl)phenyl)-2-methyl-1H-indol-5-yl)benzamide (Compound 959)

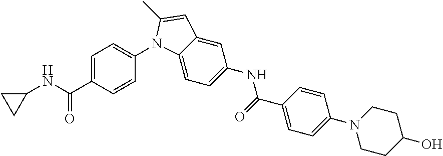 [SMILES: Cc1cc2cc(NC(=O)c3ccc(N4CCC(O)CC4)cc3)ccc2n1-c1ccc(C(=O)NC2CC2)cc1]

Compound 959 was prepared according to the procedure described in Scheme IV from 4-(5-amino-2-methyl-1H-indol-1-yl)benzoic acid, cyclopropaneamine, and 4-(4-hydroxypiperidin-1-yl)benzoic acid. [M+H]⁺ calcd for C₃₁H₃₂N₄O₃: 509.25. Found: 509.12.

### Example 860

### N-(1-(4-(Cyclopropylcarbamoyl)phenyl)-1H-indol-5-yl)-1-(2-morpholinoethyl)-1H-indole-5-carboxamide (Compound 960)

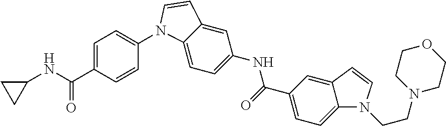 [SMILES: O=C(Nc1ccc2c(ccn2-c2ccc(C(=O)NC3CC3)cc2)c1)c1ccc2c(ccn2CCN2CCOCC2)c1]

Compound 960 was prepared according to the procedure described in Scheme IV from 4-(5-amino-1H-indol-1-yl)benzoic acid, cyclopropaneamine, and 1-(2-morpholinoethyl)-1H-indole-5-carboxylic acid. [M+H]⁺ calcd for C₃₃H₃₃N₅O₃: 548.26. Found 548.13.

### Example 861

### N-Cyclopropyl-4-(5-(4-(4-methylpiperazin-1-yl)benzamido)-1H-indol-1-yl)benzamide (Compound 961)

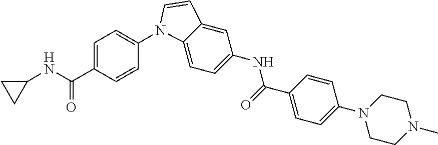 [SMILES: CN1CCN(c2ccc(C(=O)Nc3ccc4c(ccn4-c4ccc(C(=O)NC5CC5)cc4)c3)cc2)CC1]

Compound 961 was prepared according to the procedure described in Scheme IV from 4-(5-amino-1H-indol-1-yl)benzoic acid, cyclopropaneamine, and 4-(4-methylpiperazin-1-yl)benzoic acid. [M+H]⁺ calcd for C₃₀H₃₁N₅O₂: 494.25. Found 494.06.

### Example 862

### Ethyl 2-(5-(4-(5-(4-(4-hydroxypiperidin-1-yl)benzamido)-1H-indol-1-yl)benzamido)-1H-indol-1-yl)acetate (Compound 962)

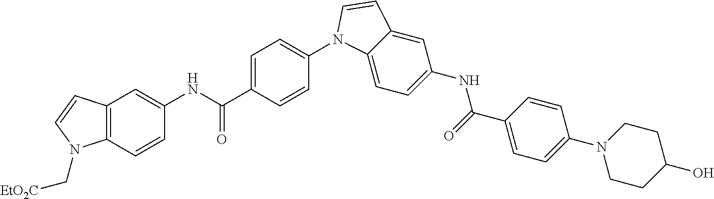 [SMILES: CCOC(=O)Cn1ccc2cc(NC(=O)c3ccc(-n4ccc5cc(NC(=O)c6ccc(N7CCC(O)CC7)cc6)ccc54)cc3)ccc21]

Compound 962 was prepared according to the procedure described in Scheme IV from ethyl 5-amino-1H-indole-1-acetate, 4-(5-amino-1H-indol-1-yl)benzoic, and 4-(4-hydroxypiperidin-1-yl)benzoic acids. [M+H]⁺ calcd for C₃₉H₃₇N₅O₅: 656.28. Found 656.23.

### Example 863

### N-(1-(4-(((1H-Indol-2-yl)methyl)carbamoyl)phenyl)-1H-indol-5-yl)-1-(2-hydroxyethyl)-1H-indole-5-carboxamide (Compound 963)

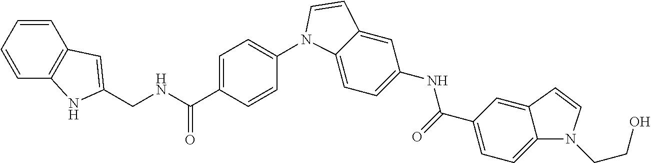 [SMILES: O=C(NCc1cc2ccccc2[nH]1)c1ccc(-n2ccc3cc(NC(=O)c4ccc5c(ccn5CCO)c4)ccc32)cc1]

Compound 963 was prepared according to the procedure described in Scheme IV from 4-(5-amino-1H-indol-1-yl)benzoic acid, 2-aminomethylindole, and 1-(2-hydroxyethyl)-1H-indole-5-carboxylic acid. ¹H NMR (500 MHz, DMSO-d₆) δ 10.14 (s, 1H), 9.42 (s, 1H), 8.31 (dd, J=1.0, 5.5 Hz, 2H), 8.14 (d, J=8.5 Hz, 2H), 8.06 (t, J=5.5 Hz, 1H), 7.87 (dd, J=1.5, 9 Hz, 1H), 7.74 (d, J=8 Hz, 1H), 7.66 (d, J=9 Hz, 2H), 7.62 (d, J=2 Hz, 1H), 7.61 (s, 1H), 7.57 (d, J=3 Hz, 1H), 7.55 (d, J=9 Hz, 1H), 7.37 (d, J=2.5 Hz, 1H), 7.11 (ddd, J=1, 7, 9 Hz, 1H) 7.03 (ddd, J=1, 7, 8.5 Hz, 1H), 6.70 (d, J=3.5 Hz, 1H), 6.56 (d, J=3.5 Hz, 1H), 4.82 (d, J=5.5 Hz, 2H), 4.35 (t, J=5.5 Hz, 2H), 3.92 (q, J=5 Hz, 2H), 3.31 (d, J=5 Hz, 1H).

### Example 864

### 2-(5-(4-(5-(4-(4-Hydroxypiperidin-1-yl)benzamido)-1H-indol-1-yl)benzamido)-1H-indol-1-yl)acetic acid (Compound 964)

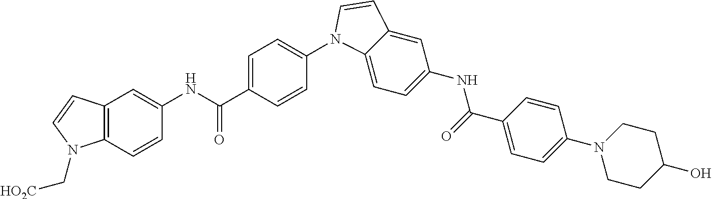 [SMILES: O=C(O)Cn1ccc2cc(NC(=O)c3ccc(-n4ccc5cc(NC(=O)c6ccc(N7CCC(O)CC7)cc6)ccc54)cc3)ccc21]

Compound 964 was prepared by hydrolysis of Compound 962. [M+H]⁺ calcd for C₃₇H₃₃N₅O₅: 628.25. Found 628.14.

### Example 865

### (±)-N-Cyclopropyl-4-(5-(4-(3-(hydroxymethyl)piperidin-1-yl)benzamido)-1H-indol-1-yl)benzamide (Compound 965)

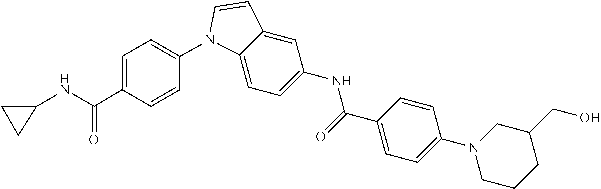 [SMILES: O=C(Nc1ccc2c(ccn2-c2ccc(C(=O)NC3CC3)cc2)c1)c1ccc(N2CCCC(CO)C2)cc1]

Compound 965 was prepared according to the procedure described in Scheme IV from 4-(5-amino-1H-indol-1-yl)benzoic acid, cyclopropaneamine, and 4-(3-(hydroxymethyl)piperazin-1-yl)benzoic acid. [M+H]⁺ calcd for C₃₁H₃₂N₄O₃: 509.25. Found: 509.05.

### Example 866

### 4-(4-Hydroxypiperidin-1-yl)-N-(1-(4-((1-(2-methoxyethyl)-2-methyl-1H-indol-5-yl)carbamoyl)phenyl)-1H-indol-5-yl)benzamide (Compound 966)

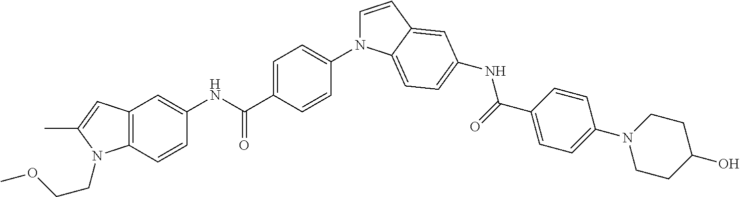 [SMILES: COCCn1c(C)cc2cc(NC(=O)c3ccc(-n4ccc5cc(NC(=O)c6ccc(N7CCC(O)CC7)cc6)ccc54)cc3)ccc21]

Compound 966 was prepared according to the procedure described in Scheme IV from ethyl 5-amino-2-methyl-1-(2-methoxyethyl)-1H-indole, 4-(5-amino-1H-indol-1-yl)benzoic, and 4-(4-hydroxypiperidin-1-yl)benzoic acids. [M+H]⁺ calcd for C₃₉H₃₉N₅O₄: 642.31. Found: 642.26.

### Example 867

### N-Cyclopropyl-4-(5-(4-(1,1-dioxidothiomorpholino)benzamido)-1H-indol-1-yl)benzamide (Compound 967)

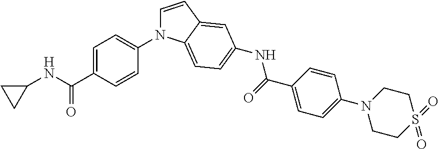 [SMILES: O=C(Nc1ccc2c(ccn2-c2ccc(C(=O)NC3CC3)cc2)c1)c1ccc(N2CCS(=O)(=O)CC2)cc1]

Compound 967 was prepared according to the procedure described in Scheme IV from 4-(5-amino-1H-indol-1-yl)benzoic acid, cyclopropaneamine, and 4-(1,1-dioxidothiomorpholino)piperazin-1-yl)benzoic acid. ¹H NMR (500 MHz, DMSO-d₆) δ 9.98 (s, 1H), 8.53 (d, J=4 Hz, 1H), 8.13 (d, J=1.5 Hz, 1H), 8.02 (d, J=8.5 Hz, 2H), 7.94 (d, J=9 Hz, 2H), 7.72 (m, 3H), 7.62 (d, J=9 Hz, 1H), 7.55 (dd, J=2, 9 Hz, 1H), 7.14 (d, J=9 Hz, 2H), 6.73 (d, J=3 Hz, 1H), 3.93 (bs, 4H), 3.14 (bs, 4H), 2.89 (m, 1H), 0.72 (m, 2H), 0.61 (m, 2H).

### Example 868

### N-(1-(4-((1H-Indol-6-yl)carbamoyl)phenyl)-1H-indol-5-yl)-4-(morpholinomethyl)benzamide (Compound 968)

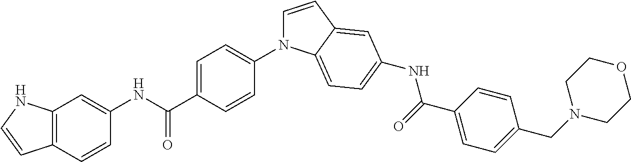 [SMILES: O=C(Nc1ccc2c(ccn2-c2ccc(C(=O)Nc3ccc4cc[nH]c4c3)cc2)c1)c1ccc(CN2CCOCC2)cc1]

Compound 968 was prepared according to the procedure described in Scheme IV from 6-aminoindole, 4-(5-amino-1H-indol-1-yl)benzoic, and 4-(morpholinomethyl)benzoic acids. [M+H]⁺ calcd for C₃₅H₃₁N₅O₃: 570.24. Found: 570.14.

### Example 869

### N-(1-(2-(Cyclopropylamino)-2-oxoethyl)-1H-indol-5-yl)-4-(5-(4-(4-hydroxypiperidin-1-yl)benzamido)-1H-indol-1-yl)benzamide (Compound 969)

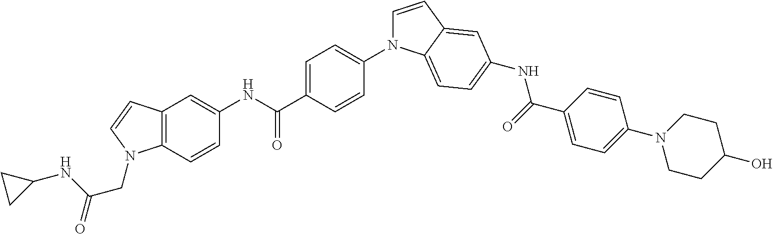 [SMILES: O=C(Cn1ccc2cc(NC(=O)c3ccc(-n4ccc5cc(NC(=O)c6ccc(N7CCC(O)CC7)cc6)ccc54)cc3)ccc21)NC1CC1]

Compound 969 was prepared by treatment of Compound 962 with cyclopropylamine. [M+H]⁺ calcd for C₄₀H₃₈N₆O₄: 667.30. Found 667.31.

### Example 870

### N-(1-(2-((Cyanomethyl)amino)-2-oxoethyl)-1H-indol-5-yl)-4-(5-(4-(4-hydroxypiperidin-1-yl)benzamido)-1H-indol-1-yl)benzamide (Compound 970)

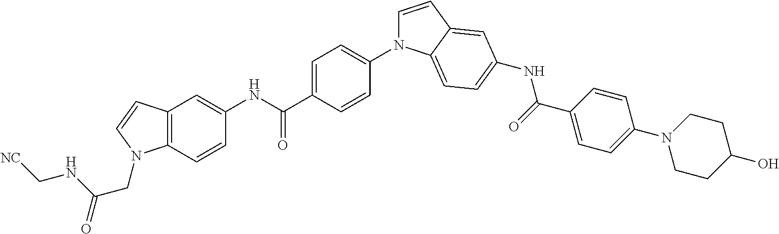 [SMILES: N#CCNC(=O)Cn1ccc2cc(NC(=O)c3ccc(-n4ccc5cc(NC(=O)c6ccc(N7CCC(O)CC7)cc6)ccc54)cc3)ccc21]

Compound 970 was prepared by treatment of Compound 962 with cyanoethylamine. [M+H]⁺ calcd for C₃₉H₃₅N₇O₄: 666.28. Found 666.23.

### Example 871

### Methyl 2-(2-(5-(4-(5-(4-(4-hydroxypiperidin-1-yl)benzamido)-1H-indol-1-yl)benzamido)-1H-indol-1-yl)acetamido)acetate (Compound 971)

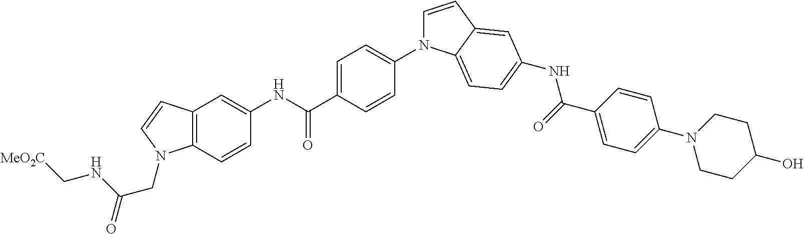 [SMILES: COC(=O)CNC(=O)Cn1ccc2cc(NC(=O)c3ccc(-n4ccc5cc(NC(=O)c6ccc(N7CCC(O)CC7)cc6)ccc54)cc3)ccc21]

Compound 971 was prepared by treatment of Compound 962 with methyl aminoacetate. [M+H]⁺ calcd for C₄₀H₃₈N₆O₆: 699.29. Found 699.25.

### Example 872

### N-(1-(4-((1H-Indol-6-yl)carbamoyl)phenyl)-1H-indol-5-yl)-1-(2-morpholinoethyl)-1H-indole-5-carboxamide (Compound 972)

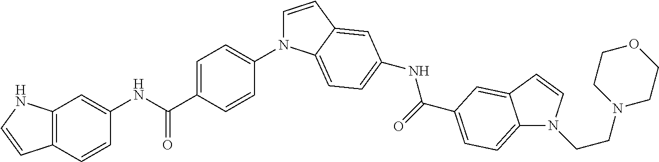 [SMILES: O=C(Nc1ccc2cc[nH]c2c1)c1ccc(-n2ccc3cc(NC(=O)c4ccc5c(ccn5CCN5CCOCC5)c4)ccc32)cc1]

Compound 972 was prepared according to the procedure described in Scheme IV from 4-(5-amino-1H-indol-1-yl)benzoic acid, 6-aminoindole, and 1-(2-morpholinoethyl)-1H-indole-5-carboxylic acid. [M+H]⁺ calcd for C₃₈H₃₄N₆O₃: 623.27. Found 623.15.

### Example 873

### Ethyl 4-(5-(4-(5-(4-(4-hydroxypiperidin-1-yl)benzamido)-1H-indol-1-yl)benzamido)-1H-indol-1-yl)butanoate (Compound 973)

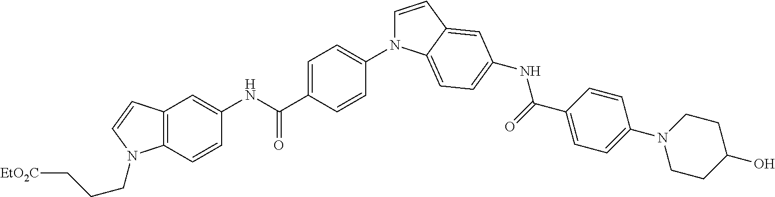 [SMILES: CCOC(=O)CCCn1ccc2cc(NC(=O)c3ccc(-n4ccc5cc(NC(=O)c6ccc(N7CCC(O)CC7)cc6)ccc54)cc3)ccc21]

Compound 973 was prepared according to the procedure described in Scheme IV from ethyl 4-(5-amino-1H-indol-1-yl)butanoate, 4-(5-amino-1H-indol-1-yl)benzoic, and 4-(4-hydroxypiperidin-1-yl)benzoic acids. [M+H]⁺ calcd for C₄₁H₄₁N₅O₅: 684.31. Found 684.19.

### Example 874

### 4-(5-(4-(5-(4-(4-Hydroxypiperidin-1-yl)benzamido)-1H-indol-1-yl)benzamido)-1H-indol-1-yl)butanoic acid (Compound 974)

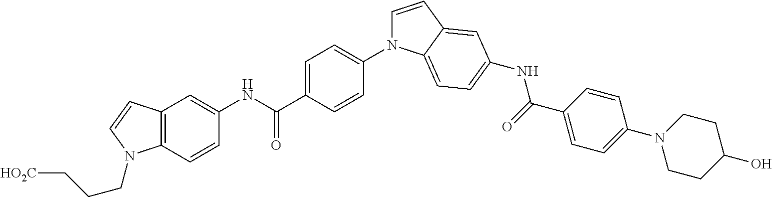 [SMILES: O=C(O)CCCn1ccc2cc(NC(=O)c3ccc(-n4ccc5cc(NC(=O)c6ccc(N7CCC(O)CC7)cc6)ccc54)cc3)ccc21]

Compound 974 was prepared hydrolysis of Compound 973. [M+H]⁺ calcd for C₃₉H₃₇N₅O₅: 656.28. Found 656.17.

### Example 875

### Methyl 2-(2-(5-(4-(5-(4-(4-hydroxypiperidin-1-yl)benzamido)-1H-indol-1-yl)benzamido)-1H-indol-1-yl)acetamido)acetate (Compound 975)

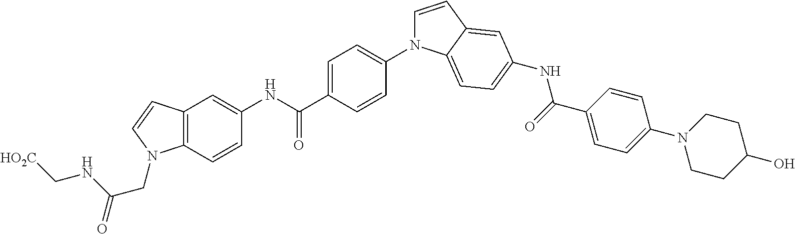 [SMILES: O=C(O)CNC(=O)Cn1ccc2cc(NC(=O)c3ccc(-n4ccc5cc(NC(=O)c6ccc(N7CCC(O)CC7)cc6)ccc54)cc3)ccc21]

Compound 975 was prepared by hydrolysis of Compound 971. [M+H]⁺ calcd for C₃₉H₃₆N₆O₆: 685.27. Found 685.14.

### Example 876

### N-(3-(4-Aminophenyl)-1H-indazol-6-yl)-4-(4-hydroxypiperidin-1-yl)benzamide (Compound 976)

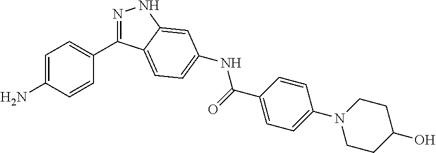 [SMILES: Nc1ccc(-c2n[nH]c3cc(NC(=O)c4ccc(N5CCC(O)CC5)cc4)ccc23)cc1]

Compound 976 was prepared according to the procedure described in Scheme IV from 6-amino-3-(4-aminophenyl)-1H-indazole, and 4-(4-hydroxypiperidin-1-yl)benzoic acid. [M+H]⁺ calcd for C₂₅H₂₅N₅O₂: 428.20. Found 428.07.

### Example 877

### N-(3-(4-(Cyclopropanecarboxamido)phenyl)-1H-indazol-6-yl)-4-(4-hydroxypiperidin-1-yl)benzamide (Compound 977)

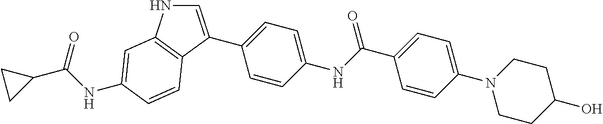 [SMILES: O=C(Nc1ccc(-c2c[nH]c3cc(NC(=O)C4CC4)ccc23)cc1)c1ccc(N2CCC(O)CC2)cc1]

Compound 977 was prepared according to the procedure described in Scheme IV from 6-amino-3-(4-aminophenyl)-1H-indazole, cyclopropanecarboxylic, and 4-(4-hydroxypiperidin-1-yl)benzoic acids. [M+H]⁺ calcd for C₃₀H₃₀N₄O₃: 495.11. Found: 495.08.

### Example 878

### N-(4-(6-(4-(4-Hydroxypiperidin-1-yl)benzamido)-1H-indazol-3-yl)phenyl)-1H-pyrrole-2-carboxamide (Compound 978)

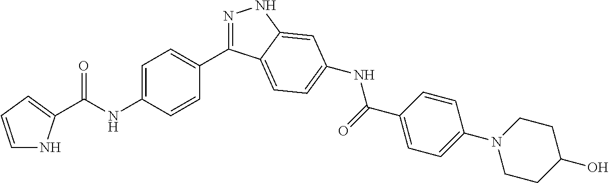 [SMILES: O=C(Nc1ccc2c(-c3ccc(NC(=O)c4ccc[nH]4)cc3)n[nH]c2c1)c1ccc(N2CCC(O)CC2)cc1]

Compound 978 was prepared according to the procedure described in Scheme IV from 6-amino-3-(4-aminophenyl)-1H-indazole, pyrrole-2-carboxylic, and 4-(4-hydroxypiperidin-1-yl)benzoic acids. [M+H]⁺ calcd for C₃₀H₂₈N₆O₃: 521.22. Found 521.07.

### Example 879

### N-(3-(4-((1H-Indol-6-yl)carbamoyl)phenyl)-1H-indol-6-yl)-1-methyl-1H-pyrrole-2-carboxamide (Compound 979)

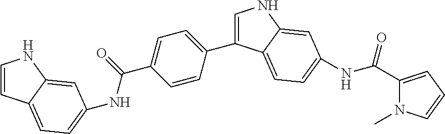 [SMILES: Cn1cccc1C(=O)Nc1ccc2c(-c3ccc(C(=O)Nc4ccc5cc[nH]c5c4)cc3)c[nH]c2c1]

Compound 979 was prepared according to the procedure described in Scheme IV from 4-(6-amino-1H-indol-3-yl)benzoic acid, 1-methylpyrrole-2-carboxylic acid, and 6-aminoindole. [M+H]⁺ calcd for C₂₉H₂₃N₅O₂: 474.05. Found: 474.02.

### Example 880

### N-(3-(4-(4-(4-Hydroxypiperidin-1-yl)benzamido)phenyl)-1H-indol-6-yl)-1-methyl-1H-pyrrole-2-carboxamide (Compound 980)

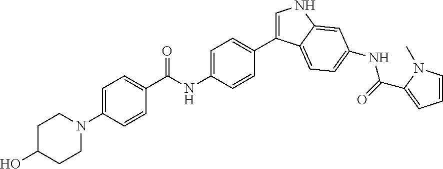 [SMILES: Cn1cccc1C(=O)Nc1ccc2c(-c3ccc(NC(=O)c4ccc(N5CCC(O)CC5)cc4)cc3)c[nH]c2c1]

Compound 980 was prepared according to the procedure described in Scheme IV from 6-amino-3-(4-aminophenyl)indole, 1-methylpyrrole-2-carboxylic, and 4-(4-hydroxypiperidin-1-yl)benzoic acids. [M+H]⁺ calcd for C₃₂H₃₁N₅O₃: 534.15. Found: 534.09.

### Example 881

### N-(3-(4-((4-(4-Hydroxypiperidin-1-yl)phenyl)carbamoyl)phenyl)-1H-indol-6-yl)-1-methyl-1H-pyrrole-2-carboxamide (Compound 981)

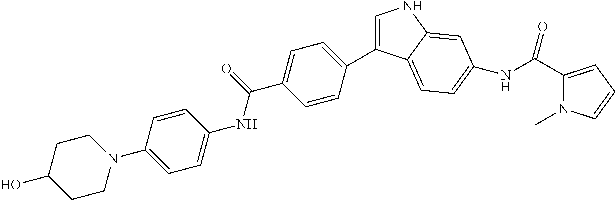 [SMILES: Cn1cccc1C(=O)Nc1ccc2c(-c3ccc(C(=O)Nc4ccc(N5CCC(O)CC5)cc4)cc3)c[nH]c2c1]

Compound 981 was prepared according to the procedure described in Scheme IV from 4-(6-amino-1H-indol-3-yl)benzoic acid, 1-methylpyrrole-2-carboxylic acid, and 4-(4-hydroxypiperidin-1-yl)aniline. [M+H]⁺ calcd for C₃₂H₃₁N₅O₃: 534.15. Found: 534.09.

### Example 882

### N-(4-(5-(4-(4-Hydroxypiperidin-1-yl)benzamido)-1H-pyrazolo[3,4-c]pyridin-1-yl)phenyl)-1-methyl-1H-pyrrole-2-carboxamide (Compound 982)

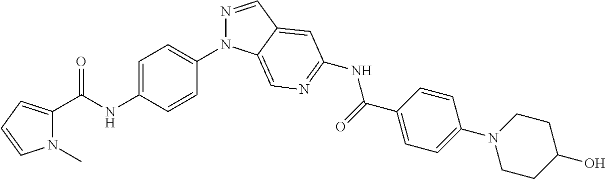 [SMILES: Cn1cccc1C(=O)Nc1ccc(-n2ncc3cc(NC(=O)c4ccc(N5CCC(O)CC5)cc4)ncc32)cc1]

Compound 982 was prepared according to the procedure described in Scheme IV from 1-(4-aminophenyl)-1H-pyrazolo[3,4-c]pyridin-5-amine, 1-methylpyrrole-2-carboxylic, and 4-(4-hydroxypiperidin-1-yl)benzoic acids. [M+H]⁺ calcd for C₃₀H₂₉N₇O₃: 536.23. Found 536.05.

### Example 883

### 2-(4-(4-((1-(4-(Cyclopropanecarboxamido)phenyl)-1H-indol-5-yl)carbamoyl)phenyl)piperazin-1-yl)acetic acid (Compound 983)

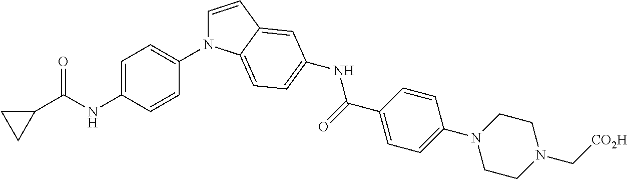 [SMILES: O=C(O)CN1CCN(c2ccc(C(=O)Nc3ccc4c(ccn4-c4ccc(NC(=O)C5CC5)cc4)c3)cc2)CC1]

Compound 983 was prepared according to the procedure described in Scheme IV from 5-amino-(4-aminophenyl)indole, cyclopropanecarboxylic acid, and 4-(4-(hydroxycarbonylmethyl)piperazin-1-yl)benzoic ester. ¹H NMR (500 MHz, Acetone-d₆) δ 9.27 (s, 1H), 8.25 (s, 1H), 8.07 (dd, J=2, 6.5 Hz, 2H), 7.95 (d, J=9 Hz, 2H), 7.82 (s, 1H), 7.60 (dd, J=2, 6.5 Hz, 2H), 7.59-7.58 (m, 3H), 7.04 (d, J=9 Hz, 2H), 6.70 (d, J=3 Hz, 1H), 3.39 (m, 3H), 2.17 (m, 1H), 1.29 (m, 8H), 0.77-0.74 (m, 2H), 0.65-0.64 (m, 2H).

### Example 884

### 4-(4-Hydroxypiperidin-1-yl)-N-(1-(4-(cyclopropylcarbamoyl)phenyl)-3-acetyl-1H-indol-5-yl)benzamide (Compound 984)

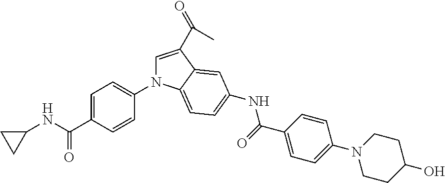 [SMILES: CC(=O)c1cn(-c2ccc(C(=O)NC3CC3)cc2)c2ccc(NC(=O)c3ccc(N4CCC(O)CC4)cc3)cc12]

Compound 984 was prepared according to the procedure described in Scheme IV from 4-(5-amino-3-acetyl-1H-indol-1-yl)benzoic acid, cyclopropaneamine, and 4-(4-hydroxypiperidin-1-yl)benzoic acid. [M+H]⁺ calcd for C₃₂H₃₂N₄O₄: 537.24. Found: 537.06.

### Example 885

### 4-(4-Hydroxypiperidin-1-yl)-N-(1-(4-(cyclopropylcarbamoyl)phenyl)-1H-indazol-5-yl)benzamide (Compound 985)

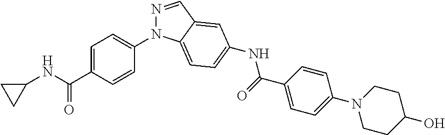 [SMILES: O=C(Nc1ccc2c(cnn2-c2ccc(C(=O)NC3CC3)cc2)c1)c1ccc(N2CCC(O)CC2)cc1]

Compound 985 was prepared according to the procedure described in Scheme IV from 4-(5-amino-1H-indazol-1-yl)benzoic acid, cyclopropaneamine, and 4-(4-hydroxypiperidin-1-yl)benzoic acid. [M+H]⁺ calcd for C₂₉H₂₉N₅O₃: 496.23. Found 496.09.

### Example 886

### 1-(4-Aminophenyl)-N-(4-(4-hydroxypiperidin-1-yl)phenyl)-1H-indazole-5-carboxamide (Compound 986)

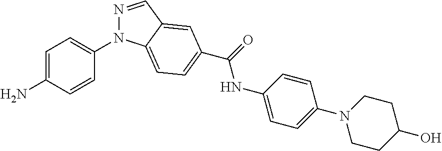 [SMILES: Nc1ccc(-n2ncc3cc(C(=O)Nc4ccc(N5CCC(O)CC5)cc4)ccc32)cc1]

Compound 986 was prepared according to the procedure described in Scheme IV from 1-(4-aminophenyl-1H-indazol-5-yl)carboxylic acid, and 4-(4-hydroxypiperidin-1-yl)aniline. [M+H]⁺ calcd for C₂₅H₂₅N₅O₂: 428.20. Found: 428.00.

### Example 887

### tert-Butyl 2-((4-(5-(4-morpholinobenzamido)-1H-indazol-1-yl)phenyl)amino)acetate (Compound 987)

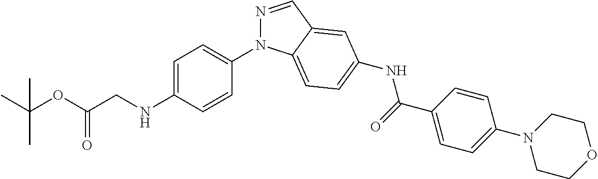 [SMILES: CC(C)(C)OC(=O)CNc1ccc(-n2ncc3cc(NC(=O)c4ccc(N5CCOCC5)cc4)ccc32)cc1]

Compound 987 was prepared according to the procedure described in Scheme IV from 5-amino-1-(4-aminophenyl)indazole, t-butyl bromoacetate, and 4-morpholinobenzoic acid. ¹H NMR (500 MHz, CDCl₃) δ 8.15 (s, 1H), 8.09 (s, 1H), 7.98 (s, 1H), 7.84 (d, J=7.5 Hz, 2H), 7.55 (d, J=9 Hz, 1H), 7.48 (d, J=10 Hz, 1H), 7.46 (d, J=7 Hz, 2H), 7.26 (s, 1H), 6.90 (d, J=7.5 Hz, 2H), 6.71 (d, J=7 Hz. 2H), 4.45 (s, 1H), 3.87-3.83 (m, 6H), 3.27-3.25 (m, 4H), 1.51 (m, 9H).

### Example 888

### N-(4-(5-(Benzyloxy)-1H-indazol-1-yl)phenyl)-4-(4-hydroxypiperidin-1-yl)benzamide (Compound 988)

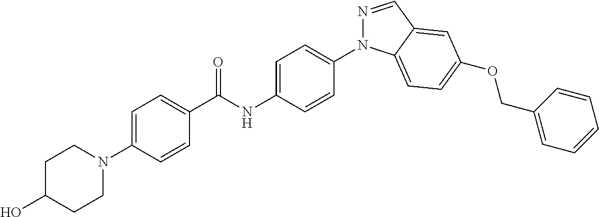 [SMILES: O=C(Nc1ccc(-n2ncc3cc(OCc4ccccc4)ccc32)cc1)c1ccc(N2CCC(O)CC2)cc1]

Compound 988 was prepared according to the procedure described in Scheme IV from 5-benzyloxy-1-(4-aminophenyl)indazole and 4-(4-hydroxypiperidin-1-yl)benzoic acid. [M+H]⁺ calcd for C₃₂H₃₀N₄O₃: 519.23. Found: 519.11.

### Example 889

### N-(4-(5-(4-Fluorobenzamido)-1H-indazol-1-yl)phenyl)isonicotinamide (Compound 989)

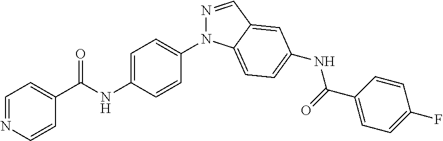 [SMILES: O=C(Nc1ccc(-n2ncc3cc(NC(=O)c4ccc(F)cc4)ccc32)cc1)c1ccncc1]

Compound 989 was prepared according to the procedure described in Scheme IV from 5-amino-1-(4-aminophenyl)indazole, isonicotinic, and 4-fluorobenzoic acids.

[M+H]⁺ calcd for C₂₆H₁₈FN₅O₂: 452.14. Found: 451.95.

### Example 890

### N-(4-(5-(Hydroxy)-1H-indazol-1-yl)phenyl)-4-(4-hydroxypiperidin-1-yl)benzamide (Compound 990)

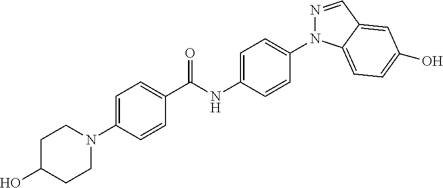 [SMILES: O=C(Nc1ccc(-n2ncc3cc(O)ccc32)cc1)c1ccc(N2CCC(O)CC2)cc1]

Compound 990 was prepared according to the procedure described in Scheme IV from 5-hydroxy-1-(4-aminophenyl)indazole and 4-(4-hydroxypiperidin-1-yl)benzoic acid. [M+H]⁺ calcd for C₂₅H₂₄N₄O₃: 429.18. Found: 429.07.

### Example 891

### 4-(Dimethylamino)-N-(4-(6-(4-(dimethylamino)benzamido)-1-(phenylsulfonyl)-1H-indol-3-yl)phenyl)benzamide (Compound 991)

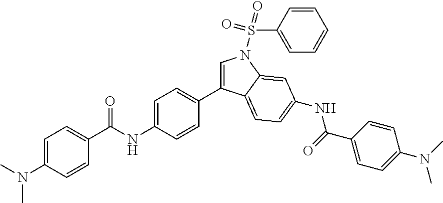

Compound 991 was prepared according to the procedure described in Scheme IV from 6-amino-3-(4-aminophenyl)-1-(phenylsulfonyl)indole and 4-dimethylaminobenzoic acid. [M+H]⁺ calcd for C₃₈H₃₅N₅O₄S: 658.31. Found: 658.19.

### Example 892

### N-(4-(5-(4-(4-Hydroxypiperidin-1-yl)benzamido)-1H-benzo[d]imidazol-1-yl)phenyl)-1H-indole-6-carboxamide (Compound 992)

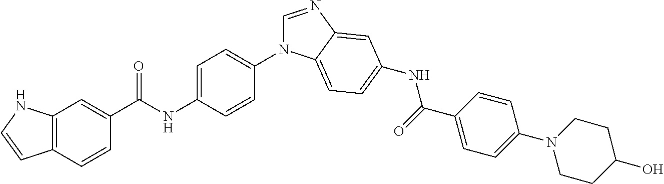 [SMILES: O=C(Nc1ccc2c(c1)ncn2-c1ccc(NC(=O)c2ccc3cc[nH]c3c2)cc1)c1ccc(N2CCC(O)CC2)cc1]

Compound 992 was prepared according to the procedure described in Scheme IV from 5-amino-1-(4-aminophenyl)benzimidazole, 6-indolecarboxylic, and 4-(4-hydroxypiperidin-1-yl)benzoic acids. [M+H]⁺ calcd for C₃₄H₃₀N₆O₃: 571.25. Found: 571.15.

### Example 893

### N-(4-(5-(Cyclopropanecarboxamido)-1H-indazol-1-yl)phenyl)-4-(4-hydroxypiperidin-1-yl)benzamide (Compound 993)

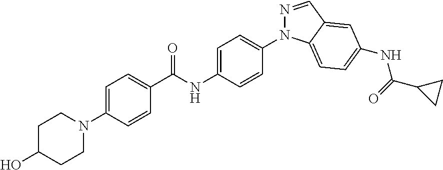 [SMILES: O=C(Nc1ccc(-n2ncc3cc(NC(=O)C4CC4)ccc32)cc1)c1ccc(N2CCC(O)CC2)cc1]

Compound 993 was prepared according to the procedure described in Scheme IV from 5-amino-1-(4-aminophenyl)indazole, cyclopropanecarboxylic, and 4-(4-hydroxypiperidin-1-yl)benzoic acids. ¹H NMR (500 MHz, Acetone-d₆) δ 9.55 (s, 1H), 9.48 (s, 1H), 8.34 (m, 1H), 8.19 (m, 1H), 8.07 (d, J=8.5 Hz, 1H), 7.94 (d, J=9 Hz, 1H), 7.86 (d, J=9 Hz, 1H), 7.80-7.69 (m, 4H), 7.61 (m, 1H), 7.04 (d, J=9 Hz, 2H), 3.80 (m, 3H), 3.11 (m, 1H), 2.09 (m, 2H), 1.94 (m, 1H), 1.80 (m, 1H), 1.60 (m, 1H), 0.93 (m, 2H), 0.80 (m, 2H).

### Example 894

### (±)-N-(4-(5-(Cyclopropanecarboxamido)-1H-indazol-1-yl)phenyl)-4-(3,4-cis-dihydroxypiperidin-1-yl)benzamide (Compound 994)

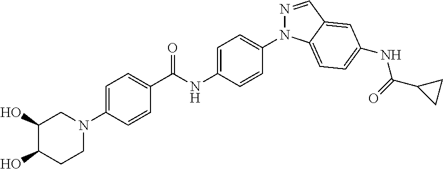 [SMILES: O=C(Nc1ccc(-n2ncc3cc(NC(=O)C4CC4)ccc32)cc1)c1ccc(N2CC[C@@H](O)[C@@H](O)C2)cc1]

Compound 994 was prepared according to the procedure described in Scheme IV from 5-amino-1-(4-aminophenyl)indazole, cyclopropanecarboxylic, and 4-(3,4-cis-dihydroxypiperidin-1-yl)benzoic acids. [M+H]⁺ calcd for C₂₉H₂₉N₅O₄: 512.22. Found 512.09.

### Example 895

### 4-(2-Hydroxyethoxy)-N-(4-(5-(4-(2-hydroxyethoxy)benzamido)-1H-indazol-1-yl)phenyl)benzamide (Compound 995)

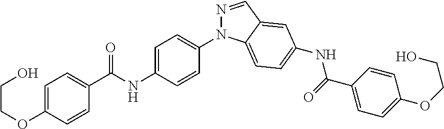 [SMILES: O=C(Nc1ccc(-n2ncc3cc(NC(=O)c4ccc(OCCO)cc4)ccc32)cc1)c1ccc(OCCO)cc1]

Compound 995 was prepared according to the procedure described in Scheme IV from 5-amino-1-(4-aminophenyl)indazole and 4-(2-hydroxyethoxy)benzoic acid. [M+H]⁺ calcd for C₃₁H₂₉N₄O₆: 553.21. Found: 553.10.

### Example 896

### N-(4-(5-(4-(4-Hydroxypiperidin-1-yl)benzamido)-1H-indazol-1-yl)phenyl)thiazole-2-carboxamide (Compound 996)

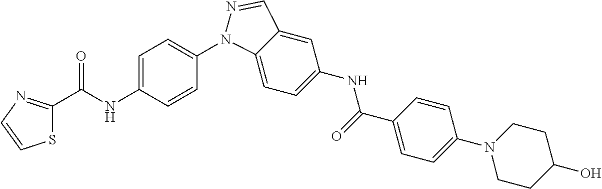 [SMILES: O=C(Nc1ccc2c(cnn2-c2ccc(NC(=O)c3nccs3)cc2)c1)c1ccc(N2CCC(O)CC2)cc1]

Compound 996 was prepared according to the procedure described in Scheme IV from 5-amino-1-(4-aminophenyl)indazole, thiozole-2-carboxylic and 4-(4-hydroxypiperidin-1-yl)benzoic acids. [M+H]⁺ calcd for C₂₉H₂₆N₆O₃S: 539.19. Found: 539.02.

### Example 897

### N-(4-(5-(4-(4-Hydroxypiperidin-1-yl)benzamido)-1H-indazol-1-yl)phenyl)-2-(methylamino)isonicotinamide (Compound 997)

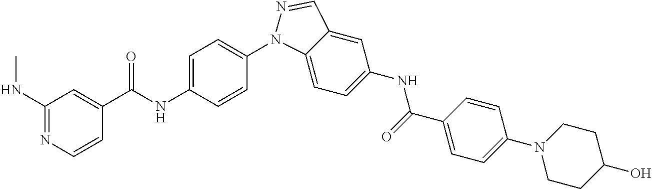 [SMILES: CNc1cc(C(=O)Nc2ccc(-n3ncc4cc(NC(=O)c5ccc(N6CCC(O)CC6)cc5)ccc43)cc2)ccn1]

Compound 997 was prepared according to the procedure described in Scheme IV from 5-amino-1-(4-aminophenyl)indazole, 2-(methylamino)isonicotinic and 4-(4-hydroxypiperidin-1-yl)benzoic acids. [M+H]⁺ calcd for C₃₂H₃₁N₇O₃: 562.26. Found: 562.13.

### Example 898

### N-(4-(5-(4-(4-Hydroxypiperidin-1-yl)benzamido)-1H-indazol-1-yl)phenyl)-2-(methyl)isonicotinamide (Compound 998)

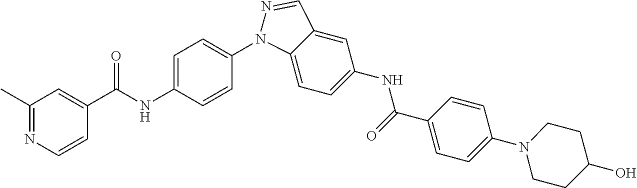 [SMILES: Cc1cc(C(=O)Nc2ccc(-n3ncc4cc(NC(=O)c5ccc(N6CCC(O)CC6)cc5)ccc43)cc2)ccn1]

Compound 998 was prepared according to the procedure described in Scheme IV from 5-amino-1-(4-aminophenyl)indazole, 2-methylisonicotinic, and 4-(4-hydroxypiperidin-1-yl)benzoic acids. [M+H]⁺ calcd for C₃₂H₃₀N₆O₃: 547.14. Found: 547.12.

### Example 899

### 4-((4-(5-(4-(4-Hydroxypiperidin-1-yl)benzamido)-1H-indazol-1-yl)phenyl)carbamoyl)pyridine 1-oxide (Compound 999)

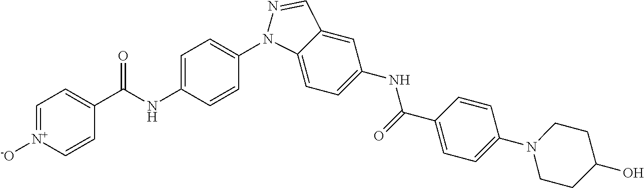

Compound 999 was prepared according to the procedure described in Scheme IV from 5-amino-1-(4-aminophenyl)indazole, 4-carboxypyridine 1-oxide, and 4-(4-hydroxypiperidin-1-yl)benzoic acid. [M+H]⁺ calcd for C₃₁H₂₈N₆O₄: 549.12. Found: 549.08.

### Example 900

### 4-(2-Hydroxyethoxy)-N-(4-(5-(4-(4-hydroxypiperidin-1-yl)benzamido)-1H-indazol-1-yl)phenyl)benzamide (Compound 1000)

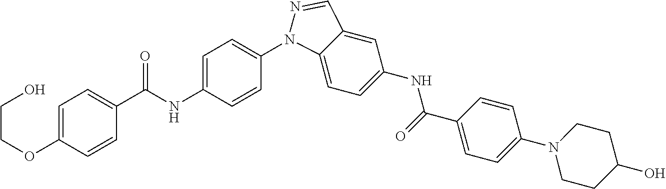 [SMILES: O=C(Nc1ccc(-n2ncc3cc(NC(=O)c4ccc(N5CCC(O)CC5)cc4)ccc32)cc1)c1ccc(OCCO)cc1]

Compound 1000 was prepared according to the procedure described in Scheme IV from 5-amino-1-(4-aminophenyl)indazole, 4-(2-hydroxyethoxy)benzoic, and 4-(4-hydroxypiperidin-1-yl)benzoic acids. [M+H]⁺ calcd for C₃₄H₃₃N₅O₅: 592.18. Found: 592.15.

### Example 901

### 4-(2-Hydroxyethoxy)-N-(1-(4-isobutyramidophenyl)-1H-indazol-5-yl)benzamide (Compound 1001)

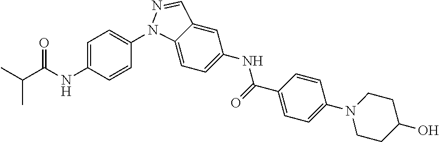 [SMILES: CC(C)C(=O)Nc1ccc(-n2ncc3cc(NC(=O)c4ccc(N5CCC(O)CC5)cc4)ccc32)cc1]

Compound 1001 was prepared according to the procedure described in Scheme IV from 5-amino-1-(4-aminophenyl)indazole, isobutyric, and 4-(4-hydroxypiperidin-1-yl)benzoic acids. [M+H]⁺ calcd for C₂₉H₃₁N₅O₃: 498.24. Found 498.11.

### Example 902

### N-(1-(4-(Cyclopent-1-enecarboxamido)phenyl)-1H-indazol-5-yl)-4-(4-hydroxypiperidin-1-yl)benzamide (Compound 1002)

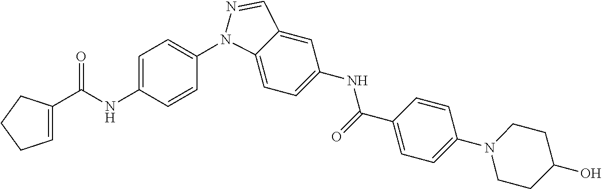 [SMILES: O=C(Nc1ccc(-n2ncc3cc(NC(=O)c4ccc(N5CCC(O)CC5)cc4)ccc32)cc1)C1=CCCC1]

Compound 1002 was prepared according to the procedure described in Scheme IV from 5-amino-1-(4-aminophenyl)indazole, cyclopentenecarboxylic, and 4-(4-hydroxypiperidin-1-yl)benzoic acids. [M+H]⁺ calcd for C₃₁H₃₁N₅O₃: 522.24. Found 522.14.

### Example 903

### 1-(2-Hydroxyethyl)-N-(4-(5-(4-(4-hydroxypiperidin-1-yl)benzamido)-1H-indazol-1-yl)phenyl)-1H-indole-5-carboxamide (Compound 1003)

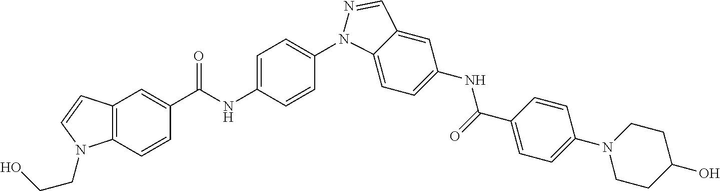 [SMILES: O=C(Nc1ccc2c(cnn2-c2ccc(NC(=O)c3ccc4c(ccn4CCO)c3)cc2)c1)c1ccc(N2CCC(O)CC2)cc1]

Compound 1003 was prepared according to the procedure described in Scheme IV from 5-amino-1-(4-aminophenyl)indazole, 1-(hydroxyethyl)indole-5-carboxylic, and 4-(4-hydroxypiperidin-1-yl)benzoic acids. [M+H]⁺ calcd for C₃₆H₃₄N₆O₄: 615.26. Found 615.20.

### Example 904

### 1-Methyl-N-(4-(5-(1-methyl-1H-indole-5-carboxamido)-1H-indazol-1-yl)phenyl)-1H-indole-5-carboxamide (Compound 1004)

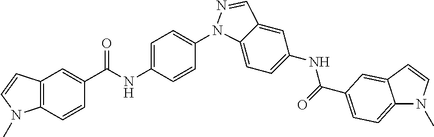 [SMILES: Cn1ccc2cc(C(=O)Nc3ccc(-n4ncc5cc(NC(=O)c6ccc7c(ccn7C)c6)ccc54)cc3)ccc21]

Compound 1004 was prepared according to the procedure described in Scheme IV from 5-amino-(4-aminophenyl)indazole and 1-methylindole-5-carboxylic acid.

[M+H]⁺ calcd for C₃₃H₂₆N₆O₂: 539.21. Found: 539.11.

### Example 905

### N-(4-(5-(4-(4-Hydroxypiperidin-1-yl)benzamido)-1H-indazol-1-yl)phenyl)-1H-pyrrolo[3,2-b]pyridine-6-carboxamide (Compound 1005)

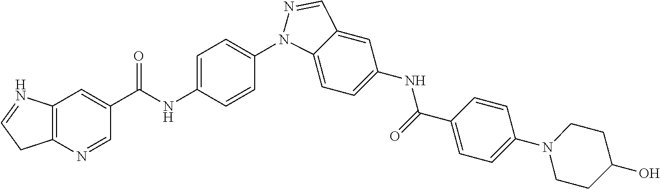

Compound 1005 was prepared according to the procedure described in Scheme IV from 5-amino-(4-aminophenyl)indazole, 1H-pyrrolo[3,2-b]pyridine-6-carboxylic, and 4-(4-hydroxypiperidin-1-yl)benzoic acids. [M+H]⁺ calcd for C₃₃H₂₉N₇O₃:572.24. Found: 572.16.

### Example 906

### 4-(4-Hydroxypiperidin-1-yl)-N-(1-(4-(3-methylbut-2-enamido)phenyl)-1H-indazol-5-yl)benzamide (Compound 1006)

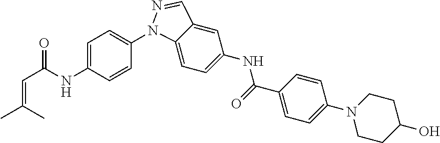 [SMILES: CC(C)=CC(=O)Nc1ccc(-n2ncc3cc(NC(=O)c4ccc(N5CCC(O)CC5)cc4)ccc32)cc1]

Compound 1006 was prepared according to the procedure described in Scheme IV from 5-amino-1-(4-aminophenyl)indazole, 3-methyl-2-butenoic, and 4-(4-hydroxypiperidin-1-yl)benzoic acids. [M+H]⁺ calcd for C₃₀H₃₁N₅O₃: 510.24. Found 510.13.

### Example 907

### N-(1-(4-(1H-Pyrrole-2-carboxamido)phenyl)-1H-indazol-5-yl)-1-methyl-1H-indole-5-carboxamide (Compound 1007)

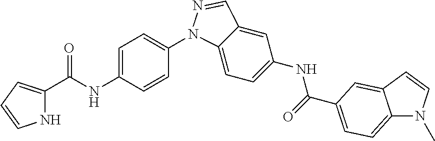 [SMILES: Cn1ccc2cc(C(=O)Nc3ccc4c(cnn4-c4ccc(NC(=O)c5ccc[nH]5)cc4)c3)ccc21]

Compound 1007 was prepared according to the procedure described in Scheme IV from 5-amino-1-(4-aminophenyl)indazole, 2-pyrrolecarboxylic, and 1-methylindole-5-carboxylic acids. [M+H]⁺ calcd for C₂₈H₂₂N₆O₂: 475.18. Found: 474.97.

### Example 908

### 1-(2-Hydroxyethyl)-N-(1-(4-(1-methyl-1H-indole-5-carboxamido)phenyl)-1H-indazol-5-yl)-1H-indole-5-carboxamide (Compound 1008)

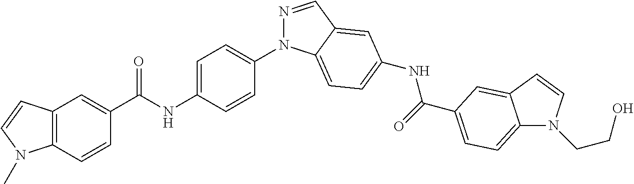 [SMILES: Cn1ccc2cc(C(=O)Nc3ccc(-n4ncc5cc(NC(=O)c6ccc7c(ccn7CCO)c6)ccc54)cc3)ccc21]

Compound 1008 was prepared according to the procedure described in Scheme IV from 5-amino-1-(4-aminophenyl)indazole, 1-(2-hydroxyethyl)indole-5-carboxylic, and 1-methylindole-5-carboxylic acids. ¹H NMR (500 MHz, Acetone-d₆) δ 9.67 (s, 1H), 9.59 (s, 1H), 8.55 (s, 1H), 8.35 (m, 2H), 8.26 (s, 1H), 8.14 (d, J=8 Hz, 2H), 7.90 (m, 4H), 7.81 (d, J=8.5 Hz, 2H), 7.59 (d, J=8.5 Hz, 1H), 7.53 (d, J=9 Hz, 1H), 7.44 (d, J=2.5 Hz, 1H), 7.36 (d, J=3 Hz, 1H), 6.60 (m, 2H), 4.37 (t, J=5.5 Hz, 2H), 3.94 (t, J=3.5 Hz, 2H), 3.91 (s, 3H).

### Example 909

### N-(1-(4-(1H-Pyrrole-2-carboxamido)phenyl)-1H-indazol-5-yl)-1-(2-hydroxyethyl)-1H-indole-5-carboxamide (Compound 1009)

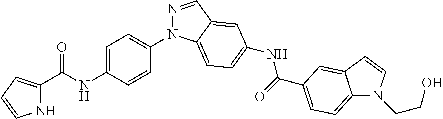 [SMILES: O=C(Nc1ccc2c(cnn2-c2ccc(NC(=O)c3ccc[nH]3)cc2)c1)c1ccc2c(ccn2CCO)c1]

Compound 1009 was prepared according to the procedure described in Scheme IV from 5-amino-1-(4-aminophenyl)indazole, 2-pyrrolecarboxylic, and 1-(2-hydroxyethyl)indole-5-carboxylic acids. [M+H]⁺ calcd for C₂₉H₂₄N₆O₃: 505.19. Found 505.10.

### Example 910

### tert-Butyl (4-((4-(5-(4-fluorobenzamido)-1H-indazol-1-yl)phenyl)carbamoyl)phenyl)carbamate (Compound 1010)

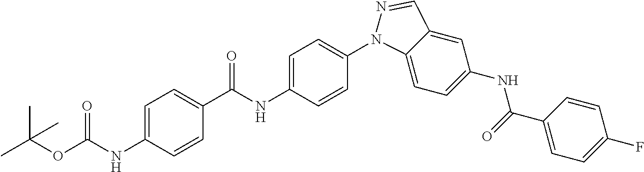 [SMILES: CC(C)(C)OC(=O)Nc1ccc(C(=O)Nc2ccc(-n3ncc4cc(NC(=O)c5ccc(F)cc5)ccc43)cc2)cc1]

Compound 1010 was prepared according to the procedure described in Scheme IV from 5-amino-(4-aminophenyl)indazole, 4-(tert-butoxycarbamoyl)benzoic, and 4-fluorobenzoic acids. [M+H]⁺ calcd for C₃₂H₂₈FN₅O₄: 566.21. Found 566.02.

### Example 911

### N-(4-(5-(4-(4-Hydroxypiperidin-1-yl)benzamido)-1H-indazol-1-yl)phenyl)pyridazine-4-carboxamide (Compound 1011)

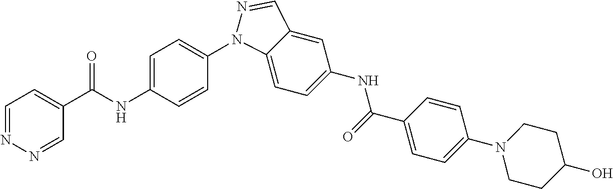 [SMILES: O=C(Nc1ccc2c(cnn2-c2ccc(NC(=O)c3ccnnc3)cc2)c1)c1ccc(N2CCC(O)CC2)cc1]

Compound 1011 was prepared according to the procedure described in Scheme IV from 5-amino-1-(4-aminophenyl)indazole, pyridazine-4-carboxylic, and 4-(4-hydroxypiperidin-1-yl)benzoic acids. [M+H]⁺ calcd for C₃₀H₂₇N₇O₃: 534.23. Found: 534.09.

### Example 912

### N-(4-(5-(4-(4-Hydroxypiperidin-1-yl)benzamido)-1H-indazol-1-yl)phenyl)-5-methylisoxazole-3-carboxamide (Compound 1012)

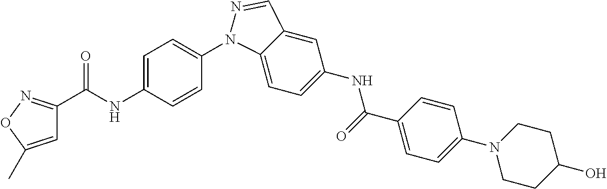 [SMILES: Cc1cc(C(=O)Nc2ccc(-n3ncc4cc(NC(=O)c5ccc(N6CCC(O)CC6)cc5)ccc43)cc2)no1]

Compound 1012 was prepared according to the procedure described in Scheme IV from 5-amino-(4-aminophenyl)indazole, 5-methylisoxazole-3-carboxylic, and 4-(4-hydroxypiperidin-1-yl)benzoic acids. [M+H]⁺ calcd for C₃₀H₂₆N₆O₄: 537.33. Found: 537.12.

### Example 913

### N-(4-(5-(4-(4-Hydroxypiperidin-1-yl)benzamido)-1H-indazol-1-yl)phenyl)-6-methylpicolinamide (Compound 1013)

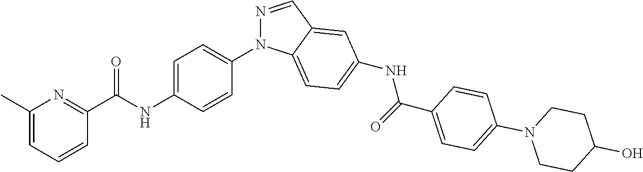 [SMILES: Cc1cccc(C(=O)Nc2ccc(-n3ncc4cc(NC(=O)c5ccc(N6CCC(O)CC6)cc5)ccc43)cc2)n1]

Compound 1013 was prepared according to the procedure described in Scheme IV from 5-amino-(4-aminophenyl)indazole, 6-methylpicolinic, and 4-(4-hydroxypiperidin-1-yl)benzoic acids. [M+H]⁺ calcd for C₃₂H₃₀N₆O₃: 547.14. Found: 547.12.

### Example 914

### N-(4-(5-(4-(4-Hydroxypiperidin-1-yl)benzamido)-1H-indazol-1-yl)phenyl)pyrimidine-4-carboxamide (Compound 1014)

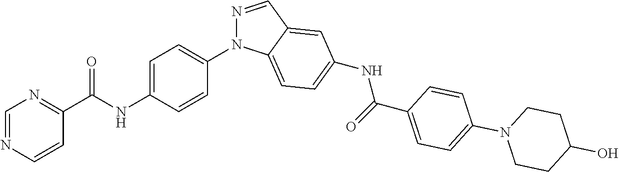 [SMILES: O=C(Nc1ccc2c(cnn2-c2ccc(NC(=O)c3ccncn3)cc2)c1)c1ccc(N2CCC(O)CC2)cc1]

Compound 1014 was prepared according to the procedure described in Scheme IV from 5-amino-1-(4-aminophenyl)indazole, pyrimidine-4-carboxylic, and 4-(4-hydroxypiperidin-1-yl)benzoic acids. [M+H]⁺ calcd for C₃₀H₂₇N₇O₃: 534.10. Found: 534.16.

### Example 915

### N-(4-(5-(4-(4-Hydroxypiperidin-1-yl)benzamido)-1H-indazol-1-yl)phenyl)-1-methyl-1H-indole-5-carboxamide (Compound 1015)

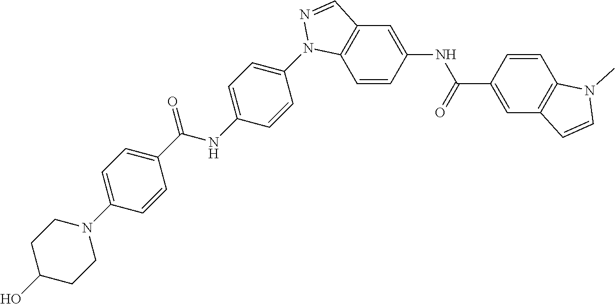 [SMILES: Cn1ccc2cc(C(=O)Nc3ccc4c(cnn4-c4ccc(NC(=O)c5ccc(N6CCC(O)CC6)cc5)cc4)c3)ccc21]

Compound 1015 was prepared according to the procedure described in Scheme IV from 5-amino-(4-aminophenyl)indazole, 1-methylindole-5-carboxylic, and 4-(4-hydroxypiperidin-1-yl)benzoic acids. [M+H]⁺ calcd for C₃₅H₃₂N₆O₃: 585.25. Found: 585.20.

### Example 916

### N-(4-(5-(4-(4-Hydroxypiperidin-1-yl)benzamido)-1H-indazol-1-yl)phenyl)-1-(3-hydroxypropyl)-1H-indole-6-carboxamide (Compound 1016)

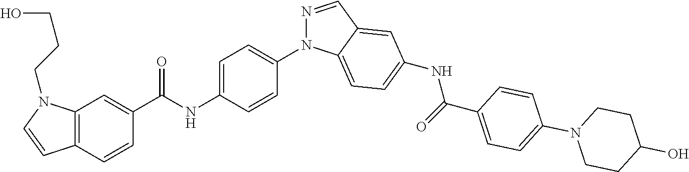 [SMILES: O=C(Nc1ccc2c(cnn2-c2ccc(NC(=O)c3ccc4ccn(CCCO)c4c3)cc2)c1)c1ccc(N2CCC(O)CC2)cc1]

Compound 1016 was prepared according to the procedure described in Scheme IV from 5-amino-1-(4-aminophenyl)indazole, 1-(3-hydroxypropyl)-1H-indole-6-carboxylic, and 4-(4-hydroxypiperidin-1-yl)benzoic acids. ¹H NMR (500 MHz, CD₃OD) δ 8.23 (s, 1H), 8.21 (s, 1H), 8.16 (s, 1H), 7.98 (d, J=8.5 Hz, 2H), 7.88 (d, J=8.5 Hz, 2H), 7.77-7.65 (m, 7H), 7.44 (d, J=3 Hz, 1H), 7.03 (d, J=9 Hz, 2H), 6.55 (d, J=3 Hz, 1H), 4.40 (t, J=6.5 Hz, 2H), 3.83-3.76 (m, 3H), 3.56 (t, J=6.5 Hz, 2H), 3.05 (t, J=10 Hz, 2H), 2.12-2.07 (m, 2H), 1.98-1.94 (m, 2H), 1.64-1.57 (m, 2H).

### Example 917

### N-(1-(4-Benzamidophenyl)-1H-indazol-5-yl)-4-(4-hydroxypiperidin-1-yl)benzamide (Compound 1017)

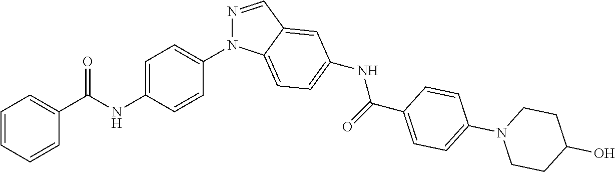 [SMILES: O=C(Nc1ccc(-n2ncc3cc(NC(=O)c4ccc(N5CCC(O)CC5)cc4)ccc32)cc1)c1ccccc1]

Compound 1017 was prepared according to the procedure described in Scheme IV from 5-amino-1-(4-aminophenyl)indazole, benzoic, and 4-(4-hydroxypiperidin-1-yl)benzoic acids. [M+H]⁺ calcd for C₃₂H₃₀N₅O₃: 532.23. Found: 532.13.

### Example 918

### N-(4-(5-(4-(4-Hydroxypiperidin-1-yl)benzamido)-1H-indazol-1-yl)phenyl)-1H-indole-2-carboxamide (Compound 1018)

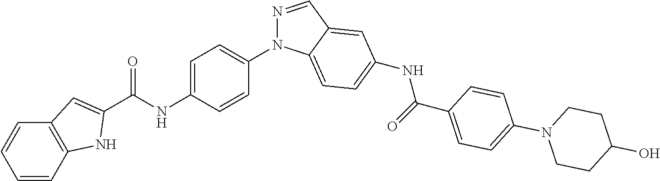 [SMILES: O=C(Nc1ccc2c(cnn2-c2ccc(NC(=O)c3cc4ccccc4[nH]3)cc2)c1)c1ccc(N2CCC(O)CC2)cc1]

Compound 1018 was prepared according to the procedure described in Scheme IV from 5-amino-1-(4-aminophenyl)indazole, indole-2-carboxylic, and 4-(4-hydroxypiperidin-1-yl)benzoic acids. [M+H]⁺ calcd for C₃₄H₃₀N₆O₃: 571.25. Found: 571.16.

### Example 919

### N-(1-(4-(2-Hydroxy-2-methylpropanamido)phenyl)-1H-indazol-5-yl)-4-(4-hydroxypiperidin-1-yl)benzamide (Compound 1019)

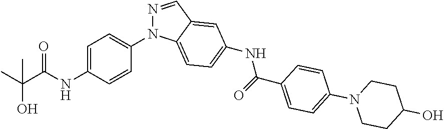 [SMILES: CC(C)(O)C(=O)Nc1ccc(-n2ncc3cc(NC(=O)c4ccc(N5CCC(O)CC5)cc4)ccc32)cc1]

Compound 1019 was prepared according to the procedure described in Scheme IV from 5-amino-1-(4-aminophenyl)indazole, 2-hydroxy-2-methylpropanoic, and 4-(4-hydroxypiperidin-1-yl)benzoic acids. [M+H]⁺ calcd for C₂₉H₃₁N₅O₄: 514.24. Found 514.11.

### Example 920

### 4-Hydroxy-N-(4-((4-(5-(4-(4-hydroxypiperidin-1-yl)benzamido)-1H-indazol-1-yl)phenyl)carbamoyl)phenyl)piperidine-1-carboxamide (Compound 1020)

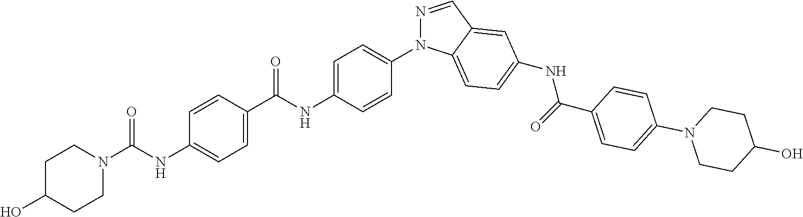 [SMILES: O=C(Nc1ccc(-n2ncc3cc(NC(=O)c4ccc(N5CCC(O)CC5)cc4)ccc32)cc1)c1ccc(NC(=O)N2CCC(O)CC2)cc1]

Compound 1020 was prepared according to the procedure described in Scheme IV from 5-amino-1-(4-aminophenyl)indazole, 4-(4-hydroxypiperidin-1-yl)carbamoylbenzoic, and 4-(4-hydroxypiperidin-1-yl)benzoic acids. [M+H]⁺ calcd for C₃₈H₃₉N₇O₅: 674.30. Found 674.33.

### Example 921

### N-(1-(4-(1-Methyl-1H-pyrrole-2-carboxamido)phenyl)-1H-indazol-5-yl)-1H-indole-5-carboxamide (Compound 1021)

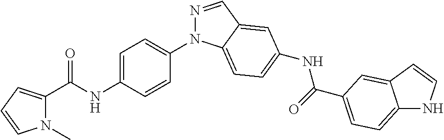 [SMILES: Cn1cccc1C(=O)Nc1ccc(-n2ncc3cc(NC(=O)c4ccc5[nH]ccc5c4)ccc32)cc1]

Compound 1021 was prepared according to the procedure described in Scheme IV from 5-amino-1-(4-aminophenyl)indazole, 1-methyl-2-pyrrolecarboxylic, and indole-5-carboxylic acids. [M+H]⁺ calcd for C₂₈H₂₂N₆O₂: 475.04. Found: 475.03.

### Example 922

### N-(1-(4-(1-Methyl-1H-pyrazole-5-carboxamido)phenyl)-1H-indazol-5-yl)-1H-indole-5-carboxamide (Compound 1022)

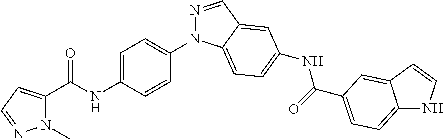 [SMILES: Cn1nccc1C(=O)Nc1ccc(-n2ncc3cc(NC(=O)c4ccc5[nH]ccc5c4)ccc32)cc1]

Compound 1022 was prepared according to the procedure described in Scheme IV from 5-amino-1-(4-aminophenyl)indazole, 1-methyl-5-pyrazolecarboxylic, and indole-5-carboxylic acids. [M+H]⁺ calcd for C₂₇H₂₁N₇O₂: 476.02. Found: 476.11.

### Example 923

### N-(1-(4-(5-Aminopicolinamido)phenyl)-1H-indazol-5-yl)-1H-indole-5-carboxamide (Compound 1023)

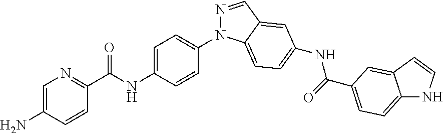 [SMILES: Nc1ccc(C(=O)Nc2ccc(-n3ncc4cc(NC(=O)c5ccc6[nH]ccc6c5)ccc43)cc2)nc1]

Compound 1023 was prepared according to the procedure described in Scheme IV from 5-amino-1-(4-aminophenyl)indazole, 5-aminopicolic, and indole-5-carboxylic acids. [M+H]⁺ calcd for C₂₈H₂₁N₇O₂: 488.04. Found: 488.06.

### Example 924

### 1-(2-Hydroxyethyl)-N-(4-(5-(1-(2-hydroxyethyl)-1H-indole-5-carboxamido)-1H-indazol-1-yl)phenyl)-1H-indole-5-carboxamide (Compound 1024)

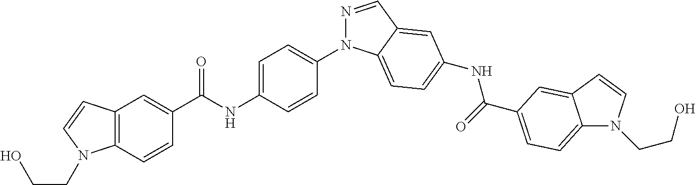 [SMILES: O=C(Nc1ccc(-n2ncc3cc(NC(=O)c4ccc5c(ccn5CCO)c4)ccc32)cc1)c1ccc2c(ccn2CCO)c1]

Compound 1024 was prepared according to the procedure described in Scheme IV from 5-amino-1-(4-aminophenyl)indazole and 1-(2-hydroxyethyl)indole-5-carboxylic.

[M+H]⁺ calcd for C₃₅H₃₁N₆O₄: 599.24. Found: 599.08.

### Example 925

### 5-Cyano-N-(4-(5-(4-(4-hydroxypiperidin-1-yl)benzamido)-1H-indazol-1-yl)phenyl)picolinamide (Compound 1025)

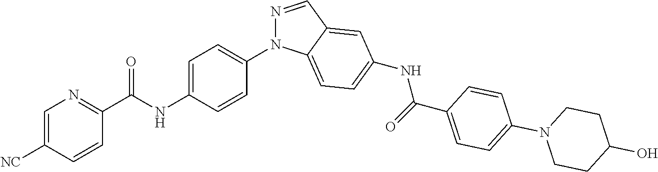 [SMILES: N#Cc1ccc(C(=O)Nc2ccc(-n3ncc4cc(NC(=O)c5ccc(N6CCC(O)CC6)cc5)ccc43)cc2)nc1]

Compound 1025 was prepared according to the procedure described in Scheme IV from 5-amino-1-(4-aminophenyl)indazole, 5-cyanopicolic, and 4-(4-hydroxypiperidin-1-yl)benzoic acids. [M+H]⁺ calcd for C₃₂H₂₇N₇O₃: 558.13. Found: 558.13.

### Example 926

### N-(4-(5-(4-(4-Hydroxypiperidin-1-yl)benzamido)-1H-indazol-1-yl)phenyl)-1H-pyrrolo[3,2-b]pyridine-5-carboxamide (Compound 1026)

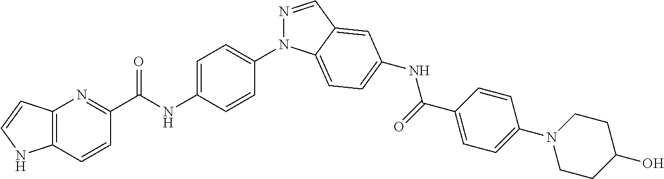 [SMILES: O=C(Nc1ccc2c(cnn2-c2ccc(NC(=O)c3ccc4[nH]ccc4n3)cc2)c1)c1ccc(N2CCC(O)CC2)cc1]

Compound 1026 was prepared according to the procedure described in Scheme IV from 5-amino-1-(4-aminophenyl)indazole, 1H-pyrrolo[3,2-b]pyridine-5-carboxylic, and 4-(4-hydroxypiperidin-1-yl)benzoic acids. [M+H]⁺ calcd for C₃₃H₂₉N₇O₃: 572.24. Found: 572.16.

### Example 927

### N-(4-(5-(4-(4-Hydroxypiperidin-1-yl)benzamido)-1H-indazol-1-yl)phenyl)-1H-pyrrolo[2,3-b]pyridine-5-carboxamide (Compound 1027)

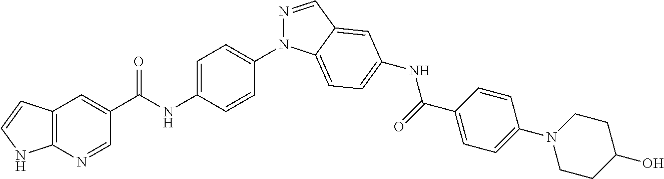 [SMILES: O=C(Nc1ccc2c(cnn2-c2ccc(NC(=O)c3cnc4[nH]ccc4c3)cc2)c1)c1ccc(N2CCC(O)CC2)cc1]

Compound 1027 was prepared according to the procedure described in Scheme IV from 5-amino-(4-aminophenyl)indazole, 1H-pyrrolo[2,3-b]pyridine-5-carboxylic, and 4-(4-hydroxypiperidin-1-yl)benzoic acids. [M+H]⁺ calcd for C₃₃H₂₉N₇O₃: 572.24. Found: 572.17.

### Example 928

### N-(4-(5-(4-(4-Hydroxypiperidin-1-yl)benzamido)-1H-indazol-1-yl)phenyl)-4-methyl-1H-pyrrole-2-carboxamide (Compound 1028)

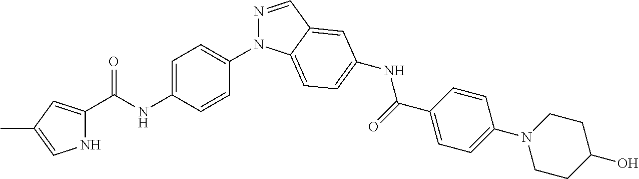 [SMILES: Cc1c[nH]c(C(=O)Nc2ccc(-n3ncc4cc(NC(=O)c5ccc(N6CCC(O)CC6)cc5)ccc43)cc2)c1]

Compound 1028 was prepared according to the procedure described in Scheme IV from 5-amino-1-(4-aminophenyl)indazole, 4-methylpyrrole-2-carboxylic, and 4-(4-hydroxypiperidin-1-yl)benzoic acids. [M+H]⁺ calcd for C₃₁H₃₀N₆O₃: 535.13. Found: 535.10.

### Example 929

### N-(4-(5-(4-(4-Hydroxypiperidin-1-yl)benzamido)-1H-indazol-1-yl)phenyl)-1H-pyrrole-3-carboxamide (Compound 1029)

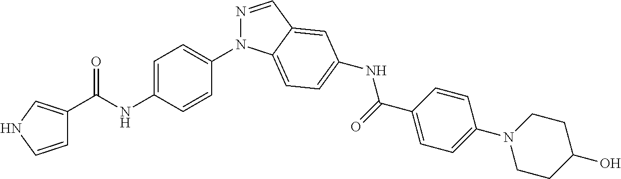 [SMILES: O=C(Nc1ccc2c(cnn2-c2ccc(NC(=O)c3cc[nH]c3)cc2)c1)c1ccc(N2CCC(O)CC2)cc1]

Compound 1029 was prepared according to the procedure described in Scheme IV from 5-amino-1-(4-aminophenyl)indazole, pyrrole-3-carboxylic, and 4-(4-hydroxypiperidin-1-yl)benzoic acids. [M+H]⁺ calcd for C₃₀H₂₈N₆O₃: 521.11. Found: 521.13.

### Example 930

### Ethyl 4-(5-(4-(4-hydroxypiperidin-1-yl)benzamido)-1H-indazol-1-yl)benzoate (Compound 1030)

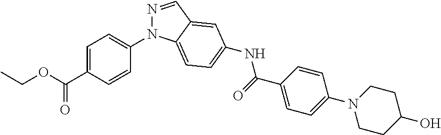 [SMILES: CCOC(=O)c1ccc(-n2ncc3cc(NC(=O)c4ccc(N5CCC(O)CC5)cc4)ccc32)cc1]

Compound 1030 was prepared according to the procedure described in Scheme IV from ethyl 4-(5-amino-1H-indazol-1-yl)benzoate and 4-(4-hydroxypiperidin-1-yl)benzoic acid. [M+H]⁺ calcd for C₂₈H₂₈N₄O₄: 485.21. Found: 485.09.

### Example 931

### N-(1-(4-((1H-Indol-6-yl)carbamoyl)phenyl)-1H-indazol-5-yl)-4-(4-hydroxypiperidin-1-yl)benzamide (Compound 1031)

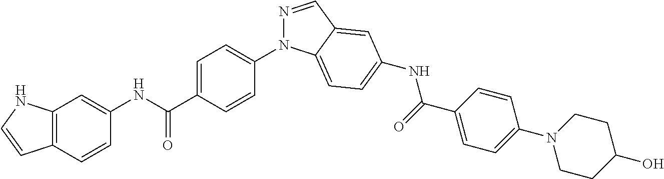 [SMILES: O=C(Nc1ccc2c(cnn2-c2ccc(C(=O)Nc3ccc4cc[nH]c4c3)cc2)c1)c1ccc(N2CCC(O)CC2)cc1]

Compound 1031 was prepared according to the procedure described in Scheme IV from 6-aminoindole, 4-(5-amino-1H-indazol-1-yl)benzoic, and 4-(4-hydroxypiperidin-1-yl)benzoic acids. [M+H]⁺ calcd for C₃₄H₃₀N₆O₃: 571.24. Found 571.16.

### Example 932

### N-(1-(4-((1H-Indol-5-yl)carbamoyl)phenyl)-1H-indazol-5-yl)-4-(4-hydroxypiperidin-1-yl)benzamide (Compound 1032)

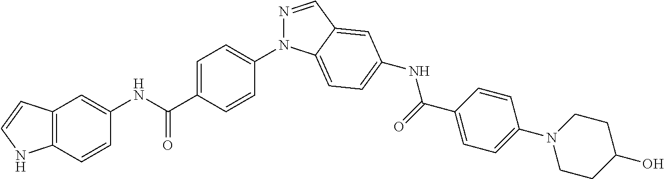 [SMILES: O=C(Nc1ccc2[nH]ccc2c1)c1ccc(-n2ncc3cc(NC(=O)c4ccc(N5CCC(O)CC5)cc4)ccc32)cc1]

Compound 1032 was prepared according to the procedure described in Scheme IV from 5-aminoindole, 4-(5-amino-1H-indazol-1-yl)benzoic, and 4-(4-hydroxypiperidin-1-yl)benzoic acids. [M+H]⁺ calcd for C₃₄H₃₀N₆O₃: 571.24. Found 571.17.

### Example 933

### 4-(4-Hydroxypiperidin-1-yl)-N-(1-(4-((2-methyl-1H-indol-5-yl)carbamoyl)phenyl)-1H-indazol-5-yl)benzamide (Compound 1033)

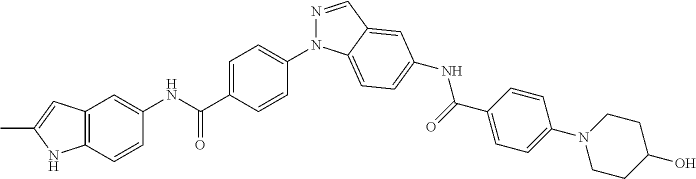 [SMILES: Cc1cc2cc(NC(=O)c3ccc(-n4ncc5cc(NC(=O)c6ccc(N7CCC(O)CC7)cc6)ccc54)cc3)ccc2[nH]1]

Compound 1033 was prepared according to the procedure described in Scheme IV from 2-methyl-5-aminoindole, 4-(5-amino-1H-indazol-1-yl)benzoic, and 4-(4-hydroxypiperidin-1-yl)benzoic acids. [M+H]⁺ calcd for C₃₅H₃₂N₆O₃: 585.25. Found 585.20.

### Example 934

### 4-(4-Hydroxypiperidin-1-yl)-N-(1-(4-((1-methyl-1H-indol-5-yl)carbamoyl)phenyl)-1H-indazol-5-yl)benzamide (Compound 1034)

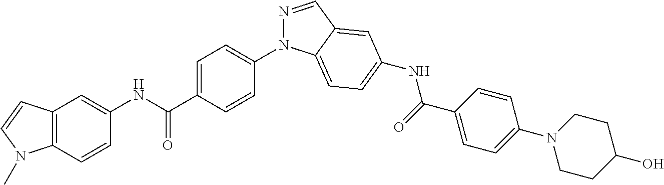 [SMILES: Cn1ccc2cc(NC(=O)c3ccc(-n4ncc5cc(NC(=O)c6ccc(N7CCC(O)CC7)cc6)ccc54)cc3)ccc21]

Compound 1034 was prepared according to the procedure described in Scheme IV from 1-methyl-5-aminoindole, 4-(5-amino-1H-indazol-1-yl)benzoic, and 4-(4-hydroxypiperidin-1-yl)benzoic acids. [M+H]⁺ calcd for C₃₅H₃₂N₆O₃: 585.25. Found 585.20.

### Example 935

### 4-(4-Hydroxypiperidin-1-yl)-N-(1-(4-(isopropylcarbamoyl)phenyl)-1H-indazol-5-yl)benzamide (Compound 1035)

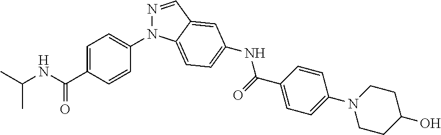 [SMILES: CC(C)NC(=O)c1ccc(-n2ncc3cc(NC(=O)c4ccc(N5CCC(O)CC5)cc4)ccc32)cc1]

Compound 1035 was prepared according to the procedure described in Scheme IV from isopropylamine, 4-(5-amino-1H-indazol-1-yl)benzoic, and 4-(4-hydroxypiperidin-1-yl)benzoic acids. [M+H]⁺ calcd for C₂₉H₃₁N₅O₃: 498.24. Found 498.11.

### Example 936

### 4-Amino-N-(4-(5-(4-fluorobenzamido)-1H-indazol-1-yl)phenyl)benzamide (Compound 1036)

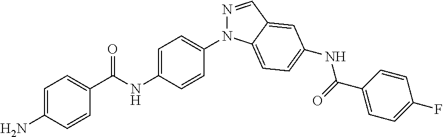 [SMILES: Nc1ccc(C(=O)Nc2ccc(-n3ncc4cc(NC(=O)c5ccc(F)cc5)ccc43)cc2)cc1]

Compound 1036 was prepared according to the procedure described in Scheme IV from 5-amino-1-(4-aminophenyl)indazole, 4-aminobenzoic, and 4-fluorobenzoic acids. [M+H]⁺ calcd for C₂₇H₂₀FN₅O₂: 466.16. Found 465.99.

### Example 937

### N-(4-(5-(4-(4-Hydroxypiperidin-1-yl)benzamido)-1H-indazol-1-yl)phenyl)-1-methyl-1H-pyrrole-2-carboxamide (Compound 1037)

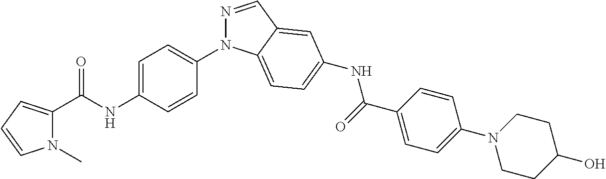 [SMILES: Cn1cccc1C(=O)Nc1ccc(-n2ncc3cc(NC(=O)c4ccc(N5CCC(O)CC5)cc4)ccc32)cc1]

Compound 1037 was prepared according to the procedure described in Scheme IV from 5-amino-1-(4-aminophenyl)indazole, 1-methylpyrrole-2-carboxylic, and 4-(4-hydroxypiperidin-1-yl)benzoic acids. [M+H]⁺ calcd for C₃₁H₃₀N₆O₃: 535.13. Found: 535.10.

### Example 938

### N-(4-(5-(4-(4-Hydroxypiperidin-1-yl)benzamido)-1H-indol-1-yl)phenyl)-1-methyl-1H-indole-5-carboxamide (Compound 1038)

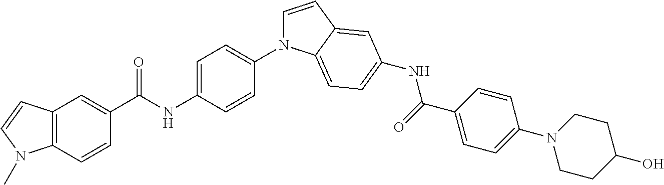 [SMILES: Cn1ccc2cc(C(=O)Nc3ccc(-n4ccc5cc(NC(=O)c6ccc(N7CCC(O)CC7)cc6)ccc54)cc3)ccc21]

Compound 1038 was prepared according to the procedure described in Scheme IV from 5-amino-(4-aminophenyl)indole, 1-methyl-1H-indole-5-carboxylic, and 4-(4-hydroxypiperidin-1-yl)benzoic acids. [M+H]⁺ calcd for C₃₆H₃₃N₅O₃: 584.26. Found: 584.12.

### Example 939

### Ethyl 4-(5-(1-(2-hydroxyethyl)-1H-indole-5-carboxamido)-1H-indazol-1-yl)benzoate (Compound 1039)

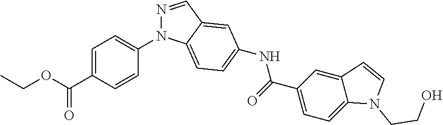 [SMILES: CCOC(=O)c1ccc(-n2ncc3cc(NC(=O)c4ccc5c(ccn5CCO)c4)ccc32)cc1]

Compound 1039 was prepared according to the procedure described in Scheme IV from ethyl 4-(5-amino-1H-indazol-1-yl)benzoate and 1-(2-hydroxyethyl)indole-5-carboxylic acid. [M+H]⁺ calcd for C₂₇H₂₄N₄O₄: 469.18. Found 469.03.

### Example 940

### N-(1-(4-(Cyclopropylcarbamoyl)phenyl)-1H-indazol-5-yl)-1-(2-hydroxyethyl)-1H-indole-5-carboxamide (Compound 1040)

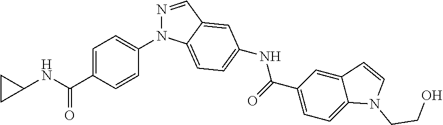 [SMILES: O=C(Nc1ccc2c(cnn2-c2ccc(C(=O)NC3CC3)cc2)c1)c1ccc2c(ccn2CCO)c1]

Compound 1040 was prepared according to the procedure described in Scheme IV from cyclopropylamine, 4-(5-amino-1H-indazol-1-yl)benzoic, and 1-(2-hydroxyethyl)indole-5-carboxylic acids. [M+H]⁺ calcd for C₂₈H₂₅N₅O₃: 480.20. Found 480.03.

### Example 941

### N-(Cyclopropylmethyl)-4-(5-(4-(4-hydroxypiperidin-1-yl)benzamido)-1H-indazol-1-yl)benzamide (Compound 1041)

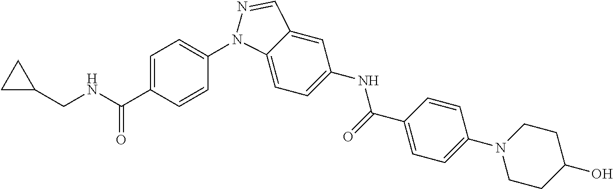 [SMILES: O=C(NCC1CC1)c1ccc(-n2ncc3cc(NC(=O)c4ccc(N5CCC(O)CC5)cc4)ccc32)cc1]

Compound 1041 was prepared according to the procedure described in Scheme IV from cyclopropylmethylamine, 4-(5-amino-1H-indazol-1-yl)benzoic, and 4-(4-hydroxypiperidin-1-yl)benzoic acids. [M+H]⁺ calcd for C₃₀H₃₁N₅O₃: 510.24. Found 510.06.

### Example 942

### 4-(4-Hydroxypiperidin-1-yl)-N-(1-(4-((5-methylthiazol-2-yl)carbamoyl)phenyl)-1H-indazol-5-yl)benzamide (Compound 1042)

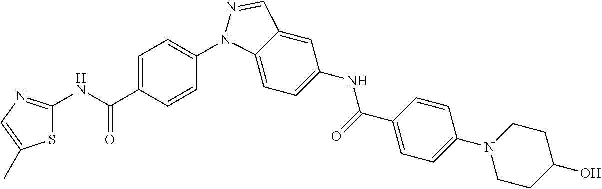 [SMILES: Cc1cnc(NC(=O)c2ccc(-n3ncc4cc(NC(=O)c5ccc(N6CCC(O)CC6)cc5)ccc43)cc2)s1]

Compound 1042 was prepared according to the procedure described in Scheme IV from 5-methylthiazol-2-ylamine, 4-(5-amino-1H-indazol-1-yl)benzoic, and 4-(4-hydroxypiperidin-1-yl)benzoic acids. [M+H]⁺ calcd for C₃₀H₂₈N₆O₃S: 553.19. Found 553.06.

### Example 943

### N-(1-(1H-Indol-5-yl)-1H-indazol-5-yl)-4-(4-hydroxypiperidin-1-yl)benzamide (Compound 1043)

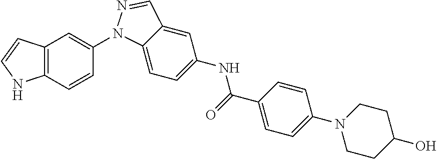 [SMILES: O=C(Nc1ccc2c(cnn2-c2ccc3[nH]ccc3c2)c1)c1ccc(N2CCC(O)CC2)cc1]

Compound 1043 was prepared according to the procedure described in Scheme IV from 5-amino-1-(indol-5-yl)indazole and 4-(4-hydroxypiperidin-1-yl)benzoic acid.

[M+H]⁺ calcd for C₂₇H₂₆N₅O₂: 452.21. Found: 452.02.

### Example 944

### 4-(4-Hydroxypiperidin-1-yl)-N-(1-(4-((2-morpholinoethyl)carbamoyl)phenyl)-1H-indazol-5-yl)benzamide (Compound 1044)

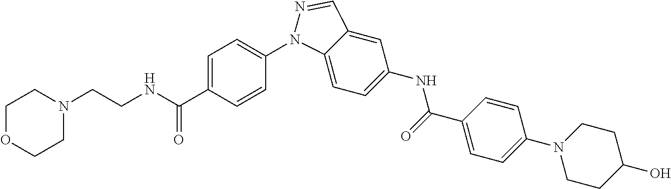 [SMILES: O=C(NCCN1CCOCC1)c1ccc(-n2ncc3cc(NC(=O)c4ccc(N5CCC(O)CC5)cc4)ccc32)cc1]

Compound 1044 was prepared according to the procedure described in Scheme IV from 2-morpholinoethylamine, 4-(5-amino-1H-indazol-1-yl)benzoic, and 4-(4-hydroxypiperidin-1-yl)benzoic acids. [M+H]⁺ calcd for C₃₂H₃₆N₆O₄: 569.28. Found: 569.33.

### Example 945

### N—(Cyanomethyl)-4-(5-(4-(4-hydroxypiperidin-1-yl)benzamido)-1H-indazol-1-yl)benzamide (Compound 1045)

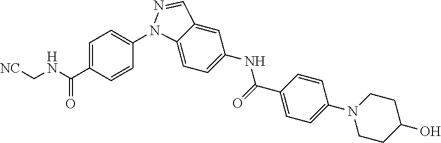 [SMILES: N#CCNC(=O)c1ccc(-n2ncc3cc(NC(=O)c4ccc(N5CCC(O)CC5)cc4)ccc32)cc1]

Compound 1045 was prepared according to the procedure described in Scheme IV from cyanomethylamine, 4-(5-amino-1H-indazol-1-yl)benzoic, and 4-(4-hydroxypiperidin-1-yl)benzoic acids. [M+H]⁺ calcd for C₂₈H₂₆N₆O₃: 495.21. Found 495.01.

### Example 946

### 4-(4-Hydroxypiperidin-1-yl)-N-(1-(4-((4-methylthiazol-2-yl)carbamoyl)phenyl)-1H-indazol-5-yl)benzamide (Compound 1046)

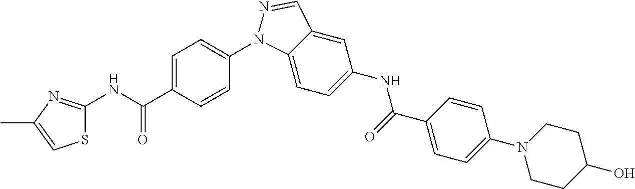 [SMILES: Cc1csc(NC(=O)c2ccc(-n3ncc4cc(NC(=O)c5ccc(N6CCC(O)CC6)cc5)ccc43)cc2)n1]

Compound 1046 was prepared according to the procedure described in Scheme IV from 4-methylthiazol-2-ylamine, 4-(5-amino-1H-indazol-1-yl)benzoic, and 4-(4-hydroxypiperidin-1-yl)benzoic acids. [M+H]⁺ calcd for C₃₀H₂₈N₆O₃S: 553.29. Found 553.06.

### Example 947

### 4-(4-Hydroxypiperidin-1-yl)-N-(1-(4-(thiazol-2-ylcarbamoyl)phenyl)-1H-indazol-5-yl)benzamide (Compound 1047)

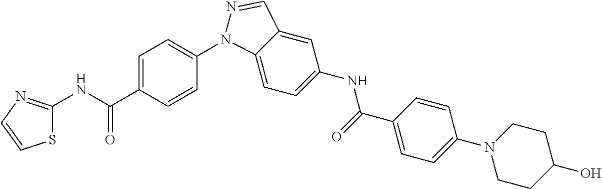 [SMILES: O=C(Nc1ccc2c(cnn2-c2ccc(C(=O)Nc3nccs3)cc2)c1)c1ccc(N2CCC(O)CC2)cc1]

Compound 1047 was prepared according to the procedure described in Scheme IV from thiazol-2-ylamine, 4-(5-amino-1H-indazol-1-yl)benzoic, and 4-(4-hydroxypiperidin-1-yl)benzoic acids. [M+H]⁺ calcd for C₂₉H₂₆N₆O₃S: 539.18. Found 539.02.

### Example 948

### tert-Butyl (1-(4-(5-(4-(4-hydroxypiperidin-1-yl)benzamido)-1H-indazol-1-yl)benzoyl)piperidin-4-yl)carbamate (Compound 1048)

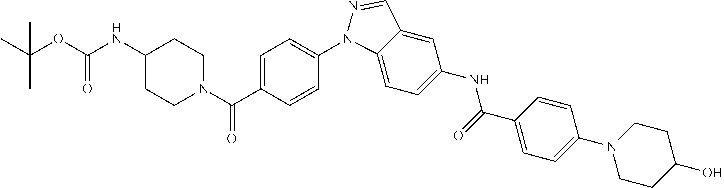 [SMILES: CC(C)(C)OC(=O)NC1CCN(C(=O)c2ccc(-n3ncc4cc(NC(=O)c5ccc(N6CCC(O)CC6)cc5)ccc43)cc2)CC1]

Compound 1048 was prepared according to the procedure described in Scheme IV from tert-butyl piperidin-4-ylcarbamate, 4-(5-amino-1H-indazol-1-yl)benzoic, and 4-(4-hydroxypiperidin-1-yl)benzoic acids. ¹H NMR (500 MHz, DMSO-d₆) δ 10.07 (s, 1H), 8.39 (d, J=0.5 Hz, 2H), 7.92 (d, J=9 Hz, 1H), 7.87 (m, 4H), 7.80 (dd, J=2, 9 Hz, 1H), 7.57 (d, J=6.5 Hz, 2H), 7.00 (d, J=9 Hz, 2H), 6.90 (d, J=7.5 Hz, 1H), 4.70 (d, J=4 Hz, 1H), 4.30 (bs, 1H), 3.70 (m, 4H), 3.52 (m, 2H), 3.00 (m, 4H), 1.80 (m, 4H), 1.44 (m, 2H), 1.37 (s, 9H).

### Example 949

### 4-(4-Hydroxypiperidin-1-yl)-N-(1-(4-(4-hydroxypiperidine-1-carbonyl)phenyl)-1H-indazol-5-yl)benzamide (Compound 1049)

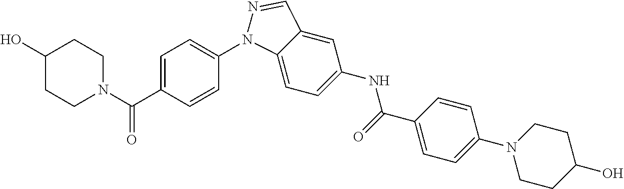 [SMILES: O=C(Nc1ccc2c(cnn2-c2ccc(C(=O)N3CCC(O)CC3)cc2)c1)c1ccc(N2CCC(O)CC2)cc1]

Compound 1049 was prepared according to the procedure described in Scheme IV from 4-hydroxypiperidine, 4-(5-amino-1H-indazol-1-yl)benzoic, and 4-(4-hydroxypiperidin-1-yl)benzoic acids. [M+H]⁺ calcd for C₃₁H₃₃N₅O₄: 540.25. Found: 540.10.

### Example 950

### tert-Butyl 4-(4-(5-(4-(4-hydroxypiperidin-1-yl)benzamido)-1H-indazol-1-yl)benzamido) piperidine-1-carboxylate (Compound 1050)

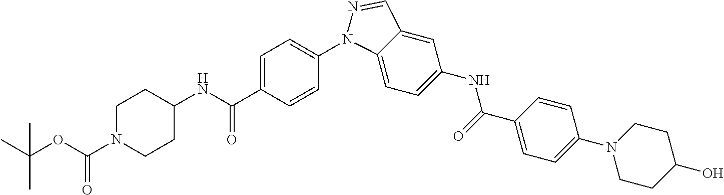 [SMILES: CC(C)(C)OC(=O)N1CCC(NC(=O)c2ccc(-n3ncc4cc(NC(=O)c5ccc(N6CCC(O)CC6)cc5)ccc43)cc2)CC1]

Compound 1050 was prepared according to the procedure described in Scheme IV from 4-amino-1-tert-butoxycarbonylpiperidine, 4-(5-amino-1H-indazol-1-yl)benzoic, and 4-(4-hydroxypiperidin-1-yl)benzoic acids. [M+H]⁺ calcd for C₃₆H₄₂N₆O₅: 639.32. Found 583.24 (-t-butyl group).

### Example 951

### 4-(4-Hydroxypiperidin-1-yl)-N-(1-(4-(morpholine-4-carbonyl)phenyl)-1H-indazol-5-yl)benzamide (Compound 1051)

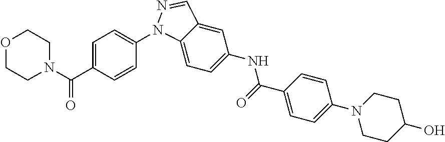 [SMILES: O=C(Nc1ccc2c(cnn2-c2ccc(C(=O)N3CCOCC3)cc2)c1)c1ccc(N2CCC(O)CC2)cc1]

Compound 1051 was prepared according to the procedure described in Scheme IV from morpholine, 4-(5-amino-1H-indazol-1-yl)benzoic, and 4-(4-hydroxypiperidin-1-yl)benzoic acids. [M+H]⁺ calcd for C₃₀H₃₁N₅O₄: 526.24. Found 526.13.

### Example 952

### 4-(4-Hydroxypiperidin-1-yl)-N-(1-(4-(piperidin-4-ylcarbamoyl)phenyl)-1H-indazol-5-yl)benzamide (Compound 1052)

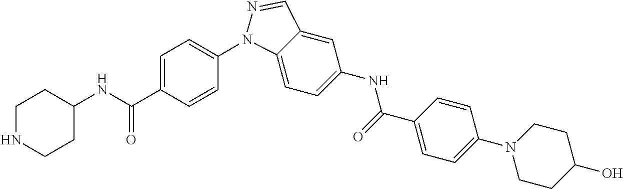 [SMILES: O=C(Nc1ccc2c(cnn2-c2ccc(C(=O)NC3CCNCC3)cc2)c1)c1ccc(N2CCC(O)CC2)cc1]

Compound 1052 was prepared by hydrolysis of Compound 1050. [M+H]⁺ calcd for C₃₁H₃₄N₆O₃: 539.27. Found 539.16.

### Example 953

### N-(1-(4-(4-Aminopiperidine-1-carbonyl)phenyl)-1H-indazol-5-yl)-4-(4-hydroxypiperidin-1-yl)benzamide (Compound 1053)

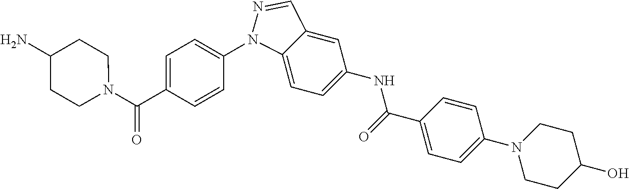 [SMILES: NC1CCN(C(=O)c2ccc(-n3ncc4cc(NC(=O)c5ccc(N6CCC(O)CC6)cc5)ccc43)cc2)CC1]

Compound 1053 was prepared by hydrolysis of Compound 1048. [M+H]⁺ calcd for C₃₁H₃₄N₆O₃: 539.27. Found 539.16.

### Example 954

### N-Benzyl-4-(5-(4-(4-hydroxypiperidin-1-yl)benzamido)-1H-indazol-1-yl)benzamide (Compound 1054)

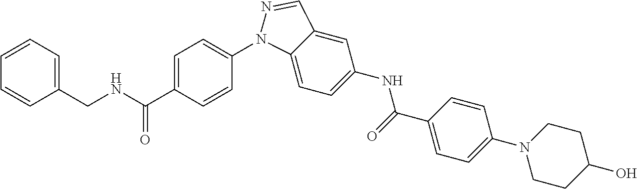 [SMILES: O=C(NCc1ccccc1)c1ccc(-n2ncc3cc(NC(=O)c4ccc(N5CCC(O)CC5)cc4)ccc32)cc1]

Compound 1054 was prepared according to the procedure described in Scheme IV from benzylamine, 4-(5-amino-1H-indazol-1-yl)benzoic, and 4-(4-hydroxypiperidin-1-yl)benzoic acids. [M+H]⁺ calcd for C₃₃H₃₁N₅O₃: 546.24. Found 546.11.

### Example 955

### N-(1-(4-(Cyclopropylcarbamoyl)phenyl)-1H-indazol-5-yl)-1-(2-hydroxyethyl)-1H-indole-6-carboxamide (Compound 1055)

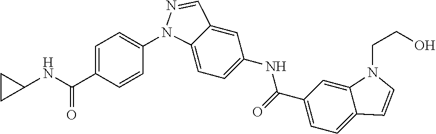 [SMILES: O=C(Nc1ccc2c(cnn2-c2ccc(C(=O)NC3CC3)cc2)c1)c1ccc2ccn(CCO)c2c1]

Compound 1055 was prepared according to the procedure described in Scheme IV from cyclopropylamine, 4-(5-amino-1H-indazol-1-yl)benzoic, and 1-(2-hydroxyethyl)indole-6-carboxylic acids. [M+H]⁺ calcd for C₂₈H₂₅N₅O₃: 480.20. Found: 480.03.

### Example 956

### 1-(2-Hydroxyethyl)-N-(1-(4-((1-methyl-1H-indol-5-yl)carbamoyl)phenyl)-1H-indazol-5-yl)-1H-indole-5-carboxamide (Compound 1056)

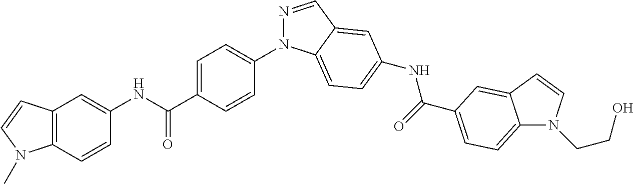 [SMILES: Cn1ccc2cc(NC(=O)c3ccc(-n4ncc5cc(NC(=O)c6ccc7c(ccn7CCO)c6)ccc54)cc3)ccc21]

Compound 1056 was prepared according to the procedure described in Scheme IV from 5-amino-1-methylindole, 4-(5-amino-1H-indazol-1-yl)benzoic, and 1-(2-hydroxyethyl)indole-5-carboxylic acids. [M+H]⁺ calcd for C₃₄H₂₈N₆O₃: 569.22. Found 569.13.

### Example 957

### N-Cyclopropyl-4-(5-(4-(4-hydroxypiperidin-1-yl)benzamido)-1H-benzo[d]imidazol-1-yl)benzamide (Compound 1057)

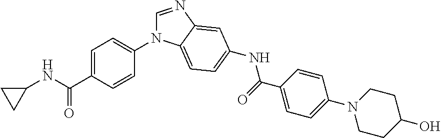 [SMILES: O=C(Nc1ccc2c(c1)ncn2-c1ccc(C(=O)NC2CC2)cc1)c1ccc(N2CCC(O)CC2)cc1]

Compound 1057 was prepared according to the procedure described in Scheme IV from cyclopropylamine, 4-(5-amino-1H-benzo[d]imidazol-1-yl)benzoic, and 4-(4-hydroxypiperidin-1-yl)benzoic acids. [M+H]⁺ calcd for C₂₉H₂₉N₅O₃: 496.24. Found: 496.09.

### Example 958

### N-Cyclopropyl-4-(5-(4-(4-hydroxypiperidinn-1-yl)benzamido)-1H-indol-1-yl)benzamide (Compound 1058)

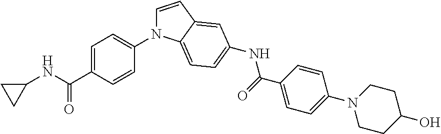 [SMILES: O=C(Nc1ccc2c(ccn2-c2ccc(C(=O)NC3CC3)cc2)c1)c1ccc(N2CCC(O)CC2)cc1]

Compound 1058 was prepared according to the procedure described in Scheme IV from cyclopropylamine, 4-(5-amino-1H-indol-1-yl)benzoic, and 4-(4-hydroxypiperidin-1-yl)benzoic acids. [M+H]⁺ calcd for C₃₀H₃₀N₄O₃: 495.23. Found: 495.14.

### Example 959

### N-(1-(4-(Cyclopropylcarbamoyl)phenyl)-1H-benzo[d]imidazol-5-yl)-1H-indole-6-carboxamide (Compound 1059)

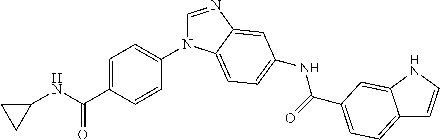 [SMILES: O=C(Nc1ccc2c(c1)ncn2-c1ccc(C(=O)NC2CC2)cc1)c1ccc2cc[nH]c2c1]

Compound 1059 was prepared according to the procedure described in Scheme IV from cyclopropylamine, 4-(5-amino-1H-benzo[d]imidazol-1-yl)benzoic, and 6-indolecarboxylic acids. [M+H]⁺ calcd for C₂₆H₂₁N₅O₂: 436.18. Found: 436.03.

### Example 960

### N-(1-(4-(Cyclopropylcarbamoyl)phenyl)-1H-benzo[d]imidazol-5-yl)-1H-indole-2-carboxamide (Compound 1060)

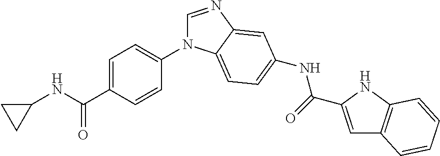 [SMILES: O=C(NC1CC1)c1ccc(-n2cnc3cc(NC(=O)c4cc5ccccc5[nH]4)ccc32)cc1]

Compound 1060 was prepared according to the procedure described in Scheme IV from cyclopropylamine, 4-(5-amino-1H-benzo[d]imidazol-1-yl)benzoic, and 2-indolecarboxylic acids. [M+H]⁺ calcd for C₂₆H₂₁N₅O₂: 436.18. Found: 436.03.

### Example 961

### N-(3-(4-(1H-Pyrrole-2-carboxamido)phenyl)-1H-indol-6-yl)-1-methyl-1H-indole-5-carboxamide (Compound 1061)

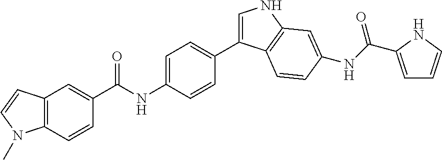 [SMILES: Cn1ccc2cc(C(=O)Nc3ccc(-c4c[nH]c5cc(NC(=O)c6ccc[nH]6)ccc45)cc3)ccc21]

Compound 1061 was prepared according to the procedure described in Scheme IV from 6-amino-3-(4-aminophenyl)indole, 2-pyrrolecarboxylic, and 1-methyl-indole-5-carboxylic acids. [M+H]⁺ calcd for C₂₉H₂₃N₅O₂: 474.05. Found: 474.09.

### Example 962

### N-(1-(2-Hydroxyethyl)-1H-indol-5-yl)-4-(5-(4-(4-hydroxypiperidin-1-yl)benzamido)-1H-indazol-1-yl)benzamide (Compound 1062)

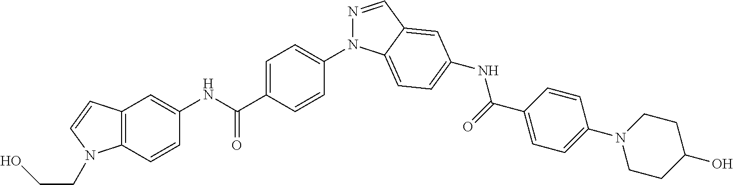 [SMILES: O=C(Nc1ccc2c(ccn2CCO)c1)c1ccc(-n2ncc3cc(NC(=O)c4ccc(N5CCC(O)CC5)cc4)ccc32)cc1]

Compound 1062 was prepared according to the procedure described in Scheme IV from 5-amino-1-(2-hydroxyethyl)indole, 4-(5-amino-1H-indazol-1-yl)benzoic, and 4-(4-hydroxypiperidin-1-yl)benzoic acids. [M+H]⁺ calcd for C₃₆H₃₄N₆O₄: 615.26. Found 615.18.

### Example 963

### 4-(4-Hydroxypiperidin-1-yl)-N-(1-(4-((1-methyl-1H-indol-5-yl)carbamoyl)phenyl)-1H-indol-5-yl)benzamide (Compound 1063)

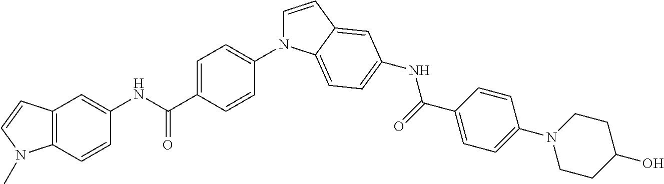 [SMILES: Cn1ccc2cc(NC(=O)c3ccc(-n4ccc5cc(NC(=O)c6ccc(N7CCC(O)CC7)cc6)ccc54)cc3)ccc21]

Compound 1063 was prepared according to the procedure described in Scheme IV from 5-amino-methylindole, 4-(5-amino-1H-indol-1-yl)benzoic, and 4-(4-hydroxypiperidin-1-yl)benzoic acids. [M+H]⁺ calcd for C₃₆H₃₃N₅O₃: 584.27. Found: 584.19.

### Example 964

### N-(4-(6-(4-(4-Hydroxypiperidin-1-yl)benzamido)-1H-indol-3-yl)phenyl)-1-methyl-1H-indole-5-carboxamide (Compound 1064)

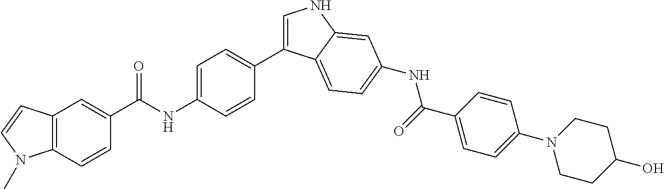 [SMILES: Cn1ccc2cc(C(=O)Nc3ccc(-c4c[nH]c5cc(NC(=O)c6ccc(N7CCC(O)CC7)cc6)ccc45)cc3)ccc21]

Compound 1064 was prepared according to the procedure described in Scheme IV from 6-amino-3-(4-aminophenyl)indole, 1-methylindole-5-carboxylic, and 4-(4-hydroxypiperidin-1-yl)benzoic acids. [M+H]⁺ calcd for C₃₆H₃₃N₅O₃: 584.21. Found: 584.19.

### Example 965

### 4-(4-Hydroxypiperidin-1-yl)-N-(1-(4-((1-(2-morpholinoethyl)-1H-indol-5-yl)carbamoyl)phenyl)-1H-indazol-5-yl)benzamide (Compound 1065)

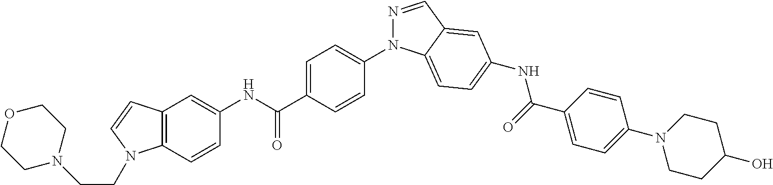 [SMILES: O=C(Nc1ccc2c(ccn2CCN2CCOCC2)c1)c1ccc(-n2ncc3cc(NC(=O)c4ccc(N5CCC(O)CC5)cc4)ccc32)cc1]

Compound 1065 was prepared according to the procedure described in Scheme IV from 5-amino-1-(2-morpholinoethyl)indole, 4-(5-amino-1H-indazol-1-yl)benzoic, and 4-(4-hydroxypiperidin-1-yl)benzoic acids. [M+H]⁺ calcd for C₄₀H₄₁N₇O₄: 684.32. Found 684.32.

### Example 966

### tert-Butyl 2-(5-(4-(5-(4-(4-hydroxypiperidin-1-yl)benzamido)-1H-indazol-1-yl)benzamido)-1H-indol-1-yl)acetate (Compound 1066)

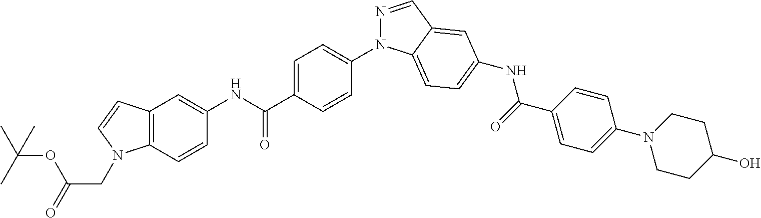 [SMILES: CC(C)(C)OC(=O)Cn1ccc2cc(NC(=O)c3ccc(-n4ncc5cc(NC(=O)c6ccc(N7CCC(O)CC7)cc6)ccc54)cc3)ccc21]

Compound 1066 was prepared according to the procedure described in Scheme IV from tert-butyl 5-aminoindol-1-ylacetate, 4-(5-amino-1H-indazol-1-yl)benzoic, and 4-(4-hydroxypiperidin-1-yl)benzoic acids. [M+H]⁺ calcd for C₄₀H₄₀N₆O₅: 685.31. Found 685.27.

### Example 967

### 4-(4-Hydroxypiperidin-1-yl)-N-(1-(4-((1-methyl-1H-imidazo[4,5-b]pyridin-6-yl)carbamoyl)phenyl)-1H-indol-5-yl)benzamide (Compound 1067)

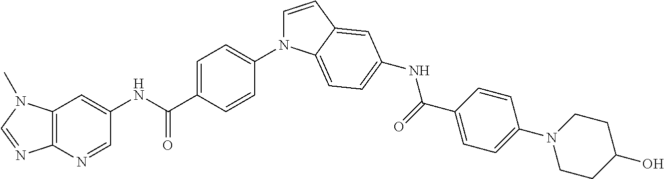 [SMILES: Cn1cnc2ncc(NC(=O)c3ccc(-n4ccc5cc(NC(=O)c6ccc(N7CCC(O)CC7)cc6)ccc54)cc3)cc21]

Compound 1067 was prepared according to the procedure described in Scheme IV from 1-methyl-1H-imidazo[4,5-b]pyridin-6-ylamine, 4-(5-amino-1H-indol-1-yl)benzoic, and 4-(4-hydroxypiperidin-1-yl)benzoic acids. [M+H]⁺ calcd for C₃₄H₃₁N₇O₃: 586.26. Found: 586.14.

### Example 968

### N-(1-(4-((1H-Pyrrolo[2,3-b]pyridin-5-yl)carbamoyl)phenyl)-1H-indol-5-yl)-4-(4-hydroxypiperidin-1-yl)benzamide (Compound 1068)

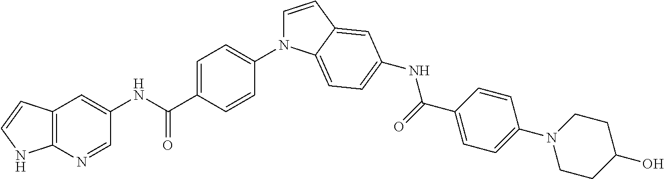 [SMILES: O=C(Nc1cnc2[nH]ccc2c1)c1ccc(-n2ccc3cc(NC(=O)c4ccc(N5CCC(O)CC5)cc4)ccc32)cc1]

Compound 1068 was prepared according to the procedure described in Scheme IV from 5-amino-1H-Pyrrolo[2,3-b]pyridine, 4-(5-amino-1H-indol-1-yl)benzoic, and 4-(4-hydroxypiperidin-1-yl)benzoic acids. [M+H]⁺ calcd for C₃₄H₃₀N₆O₃: 571.25. Found: 571.16.

### Example 969

### 4-(4-Hydroxypiperidin-1-yl)-N-(1-(4-((5-methylthiazol-2-yl)carbamoyl)phenyl)-1H-indol-5-yl)benzamide (Compound 1069)

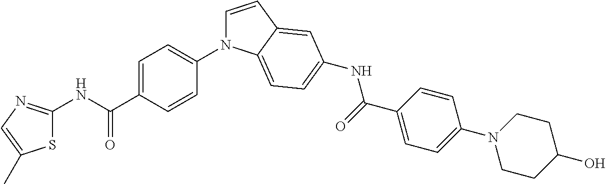 [SMILES: Cc1cnc(NC(=O)c2ccc(-n3ccc4cc(NC(=O)c5ccc(N6CCC(O)CC6)cc5)ccc43)cc2)s1]

Compound 1069 was prepared according to the procedure described in Scheme IV from 2-amino-5-methylthiazole, 4-(5-amino-1H-indol-1-yl)benzoic, and 4-(4-hydroxypiperidin-1-yl)benzoic acids. [M+H]⁺ calcd for C₃₁H₂₉N₅O₃: 552.09. Found: 552.12.

### Example 970

### N-(4-(3-cyclopropyl-5-(4-(4-hydroxypiperidin-1-yl)benzamido)-1H-indol-1-yl)phenyl)-1-methyl-1H-pyrrole-3-carboxamide (Compound 1070)

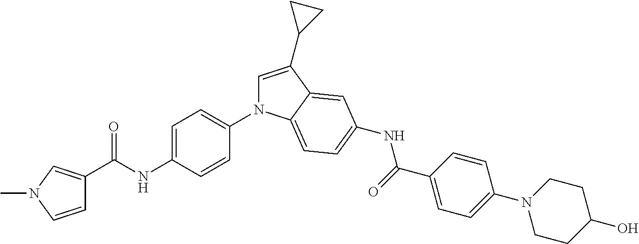 [SMILES: Cn1ccc(C(=O)Nc2ccc(-n3cc(C4CC4)c4cc(NC(=O)c5ccc(N6CCC(O)CC6)cc5)ccc43)cc2)c1]

Compound 1070 was prepared according to the procedure described in Scheme IV from 3-amino-1-methylpyrrole, 4-(5-amino-3-cyclopropyl-1H-indol-1-yl)benzoic, and 4-(4-hydroxypiperidin-1-yl)benzoic acids. ¹H NMR (400 MHz, Acetone-d₆) δ 9.13 (s, 1H), 9.12 (s, 1H), 8.08 (d, J=1.8 Hz, 1H), 8.04 (d, J=8.8 Hz, 2H), 7.93 (d, J=9 Hz, 2H), 7.45 (m, 3H), 7.40 (dd, J=1.8, 8.8 Hz, 1H), 7.02 (d, J=9 Hz, 1H), 7.02 (d, J=9 Hz, 2H), 6.75 (t, J=2.6 Hz, 1H), 6.68 (dd, J=1.8, 2.8 Hz, 1H), 6.20 (s, 1H), 3.84 (m, 2H), 3.77 (s, 3H), 3.74 (m, 2H), 3.08 (ddd, J=3.2, 9.8, 13 Hz, 2H), 1.95 (m, 2H), 1.75 (m, 1H), 1.61 (m, 2H), 0.90 (m, 2H), 0.80 (m, 2H).

### Example 971

### The following additional compounds are known and prepared according to standard literature procedures and Table 1 summarises the structure and names of the compounds.

TABLE 1

| Cpd # | Structure | Name |
|---|---|---|
| 1700 | 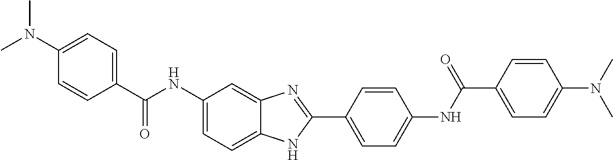 | 4-Dimethylamino-N-(4-(5-(4- dimethylaminobenzamido)-1H- benzimidazol-2- yl)phenyl)benzamide |
| 1701 | 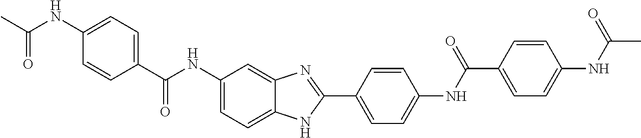 [SMILES: CC(=O)Nc1ccc(C(=O)Nc2ccc(-c3nc4cc(NC(=O)c5ccc(NC(C)=O)cc5)ccc4[nH]3)cc2)cc1] | 4-(Acetamido)-N-(4-(5-(4- acetamidobenzamido)-1H- benzimidazol-2- yl)phenyl)benzamide |
| 1702 | 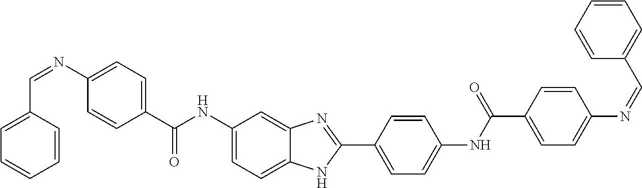 [SMILES: O=C(Nc1ccc(-c2nc3cc(NC(=O)c4ccc(/N=C\c5ccccc5)cc4)ccc3[nH]2)cc1)c1ccc(/N=C\c2ccccc2)cc1] | 4-(Benzylideneamino)-N-(4-(5- (4-benzylideneamino- benzamido)-1H-benzimidazol- 2-yl)phenyl)benzamide |
| 1703 | 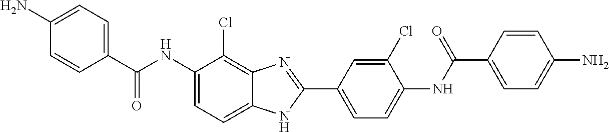 [SMILES: Nc1ccc(C(=O)Nc2ccc(-c3nc4c(Cl)c(NC(=O)c5ccc(N)cc5)ccc4[nH]3)cc2Cl)cc1] | 4-Amino-N-(4-(5-(4- aminobenzamido)-4-chloro- 1H-benzimidazol-2-yl)-2- chlorophenyl)benzamide |
| 1704 | 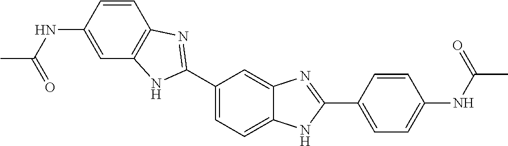 [SMILES: CC(=O)Nc1ccc(-c2nc3cc(-c4nc5ccc(NC(C)=O)cc5[nH]4)ccc3[nH]2)cc1] | N-(4-(6-Acetamido-1H,1′H- 2,5′-bibenzamidazol-2- yl)phenyl)acetamide |
| 1705 | 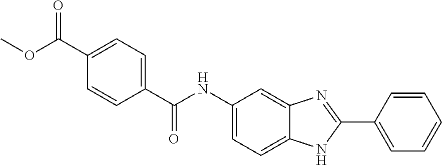 [SMILES: COC(=O)c1ccc(C(=O)Nc2ccc3[nH]c(-c4ccccc4)nc3c2)cc1] | Methyl 4-(2-phenyl-1H- benzimidazol-5-ylcarbamoyl)- benzoate |
| 1706 | 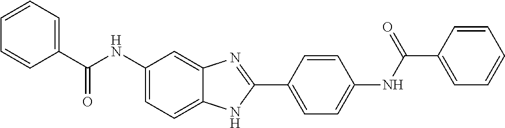 [SMILES: O=C(Nc1ccc(-c2nc3cc(NC(=O)c4ccccc4)ccc3[nH]2)cc1)c1ccccc1] | N-(4-(5-Benzamido-1H- benzimidazol-2- yl)phenyl)benzamide |
| 1707 | 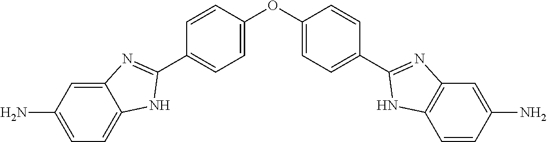 [SMILES: Nc1ccc2[nH]c(-c3ccc(Oc4ccc(-c5nc6cc(N)ccc6[nH]5)cc4)cc3)nc2c1] | 2,2′-(4,4′-Oxybis(4,1- phenylene))bis(1H- benzimidazole-5-amine) |
| 1708 | 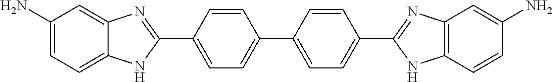 [SMILES: Nc1ccc2[nH]c(-c3ccc(-c4ccc(-c5nc6cc(N)ccc6[nH]5)cc4)cc3)nc2c1] | 2,2′-(Biphenyl-4,4′-diyl)bis(1H- benzimidazol-5-amine) |
| 1709 | 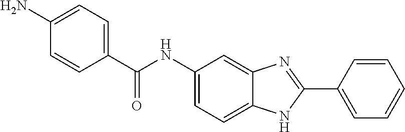 [SMILES: Nc1ccc(C(=O)Nc2ccc3[nH]c(-c4ccccc4)nc3c2)cc1] | 4-Amino-N-(2-phenyl-1H- benzimidazol-5-yl)benzamide |
| 1710 | 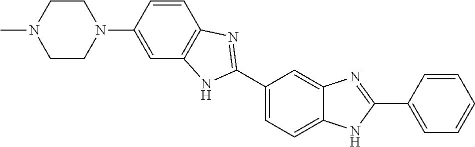 [SMILES: CN1CCN(c2ccc3nc(-c4ccc5[nH]c(-c6ccccc6)nc5c4)[nH]c3c2)CC1] | 5-(6-(4-Methylpiperazino)-1H- benzimidazol-2-yl)-2-phenyl- 1H-benzimidazole |
| 1711 | 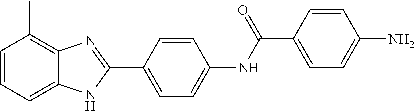 [SMILES: Cc1cccc2[nH]c(-c3ccc(NC(=O)c4ccc(N)cc4)cc3)nc12] | 4-Amino-N-(4-(4-methyl-1H- benzimidazol-2- yl)phenyl)benzamide |
| 1712 | 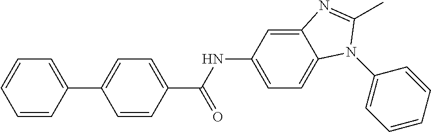 [SMILES: Cc1nc2cc(NC(=O)c3ccc(-c4ccccc4)cc3)ccc2n1-c1ccccc1] | N-(2-Methyl-1-phenyl-1H- benzimidazol-5-yl)biphenyl-4- carboxamide |
| 1713 | 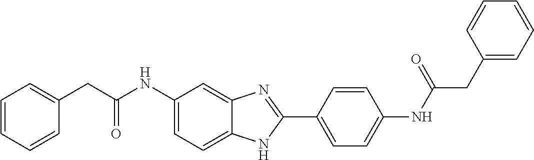 [SMILES: O=C(Cc1ccccc1)Nc1ccc(-c2nc3cc(NC(=O)Cc4ccccc4)ccc3[nH]2)cc1] | 2-Phenyl-N-(4-(5-(2- phenylacetamido)-1H- benzimidazol-2- yl)phenyl)acetamide |
| 1714 | 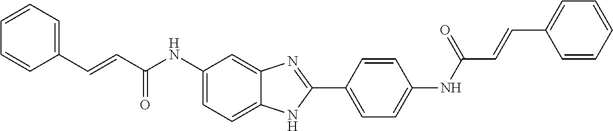 [SMILES: O=C(/C=C/c1ccccc1)Nc1ccc(-c2nc3cc(NC(=O)/C=C/c4ccccc4)ccc3[nH]2)cc1] | N-(2-(4-(3(E)- Phenylacrylamido)phenyl)-1H- benzimidazol-5-yl)-3(E)- phenylacrylamide |
| 1715 | 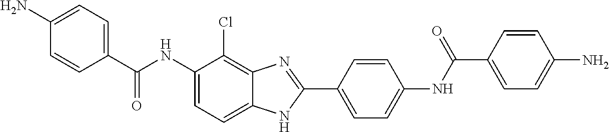 [SMILES: Nc1ccc(C(=O)Nc2ccc(-c3nc4c(Cl)c(NC(=O)c5ccc(N)cc5)ccc4[nH]3)cc2)cc1] | 4-Amino-N-(4-(5-(4- aminobenzamido)-4-chloro- 1H-benzimidazol-2- yl)phenyl)benzamide |
| 1716 | 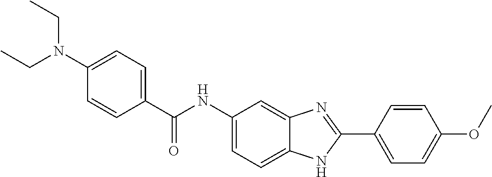 [SMILES: CCN(CC)c1ccc(C(=O)Nc2ccc3[nH]c(-c4ccc(OC)cc4)nc3c2)cc1] | 4-Diethylamino-N-(2-(4- methoxyphenyl)-1H- benzimidazol-5-yl)benzamide |
| 1717 | 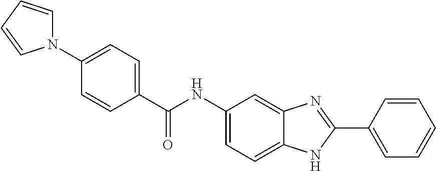 [SMILES: O=C(Nc1ccc2[nH]c(-c3ccccc3)nc2c1)c1ccc(-n2cccc2)cc1] | N-(2-Phenyl-1H-benzimidazol- 5-yl)-4-pyrrolylbenzamide |
| 1718 | 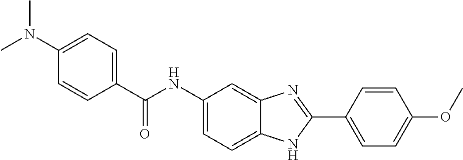 | 4-Dimethylamino-N-(2-(4- methoxyphenyl)-1H- benzimidazol-5-yl)benzamide |
| 1719 | 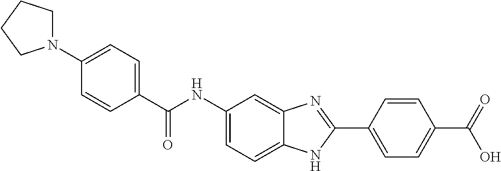 [SMILES: O=C(O)c1ccc(-c2nc3cc(NC(=O)c4ccc(N5CCCC5)cc4)ccc3[nH]2)cc1] | (4-(5-(4- Pyrrolidinobenzamido)-1H- benzimidazol-2-yl)benzoic acid |
| 1720 | 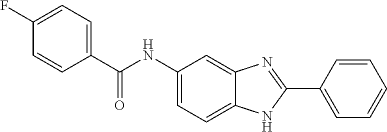 [SMILES: O=C(Nc1ccc2[nH]c(-c3ccccc3)nc2c1)c1ccc(F)cc1] | 4-Fluoro-N-(2-phenyl-1H- benzimidazol-5-yl)benzamide |
| 1721 | 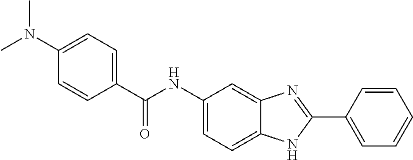 [SMILES: CN(C)c1ccc(C(=O)Nc2ccc3[nH]c(-c4ccccc4)nc3c2)cc1] | 4-Dimethylamino-N-(2-phenyl- 1H-benzimidazol-5- yl)benzamide |
| 1722 | 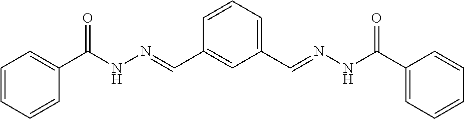 [SMILES: O=C(N/N=C/c1cccc(/C=N/NC(=O)c2ccccc2)c1)c1ccccc1] | 2,2′-(1,3- Phenylenebis(methylidene) dibenzohydrazide |
| 1723 | 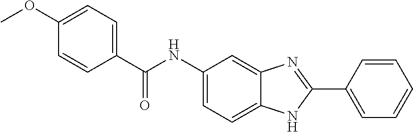 [SMILES: COc1ccc(C(=O)Nc2ccc3[nH]c(-c4ccccc4)nc3c2)cc1] | 4-Methoxy-N-(2-phenyl-1H- benzimidazol-5-yl)benzamide |
| 1724 | 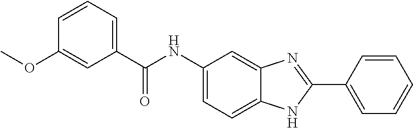 [SMILES: COc1cccc(C(=O)Nc2ccc3[nH]c(-c4ccccc4)nc3c2)c1] | 3-Methoxy-N-(2-phenyl-1H- benzimidazol-5-yl)benzamide |
| 1725 | 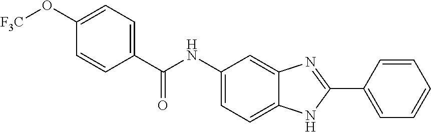 [SMILES: O=C(Nc1ccc2[nH]c(-c3ccccc3)nc2c1)c1ccc(OC(F)(F)F)cc1] | 4-Trifluoromethoxy-N-(2- phenyl-1H-benzimidazol-5- yl)benzamide |
| 1726 | 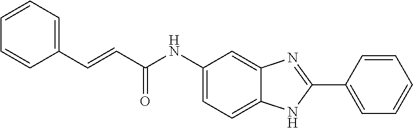 [SMILES: O=C(/C=C/c1ccccc1)Nc1ccc2[nH]c(-c3ccccc3)nc2c1] | N-(2-Phenyl-1H-benzimidazol- 5-yl)-3(E)-phenylacrylamide |
| 1727 | 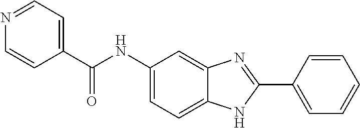 [SMILES: O=C(Nc1ccc2[nH]c(-c3ccccc3)nc2c1)c1ccncc1] | N-(2-Phenyl-1H-benzimidazol- 5-yl)isonicotinamide |
| 1728 | 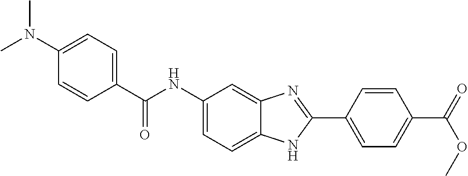 [SMILES: COC(=O)c1ccc(-c2nc3cc(NC(=O)c4ccc(N(C)C)cc4)ccc3[nH]2)cc1] | Methyl (4-(5-(4- dimethylaminobenzamido)-1H- benzimidazol-2-yl)benzoate |
| 1729 | 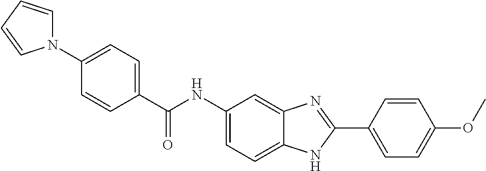 [SMILES: COc1ccc(-c2nc3cc(NC(=O)c4ccc(-n5cccc5)cc4)ccc3[nH]2)cc1] | N-(2-(4-Methoxyphenyl)-1H- benzimidazol-5-yl)-4- pyrrolylbenzamide |
| 1730 | 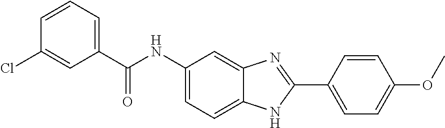 [SMILES: COc1ccc(-c2nc3cc(NC(=O)c4cccc(Cl)c4)ccc3[nH]2)cc1] | N-(2-(4-Methoxyphenyl)-1H- benzimidazol-5-yl)-3- chlorobenzamide |
| 1731 | 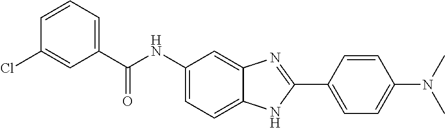 [SMILES: CN(C)c1ccc(-c2nc3cc(NC(=O)c4cccc(Cl)c4)ccc3[nH]2)cc1] | N-(2-(4-Dimethylaminophenyl)- 1H-benzimidazol-5-yl)-3- chlorobenzamide |
| 1732 | 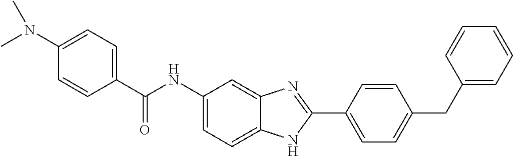 [SMILES: CN(C)c1ccc(C(=O)Nc2ccc3[nH]c(-c4ccc(Cc5ccccc5)cc4)nc3c2)cc1] | N-(2-(4-Benzylphenyl)-1H- benzimidazol-5-yl)-4- dimethylaminobenzamide |
| 1733 | 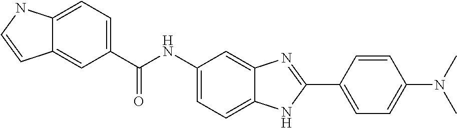 [SMILES: CN(C)c1ccc(-c2nc3cc(NC(=O)c4ccc5[nH]ccc5c4)ccc3[nH]2)cc1] | N-(2-(4-Dimethylaminophenyl)- 1H-benzimidazol-5-yl)-1H- indole-5-carboxamide |
| 1734 | 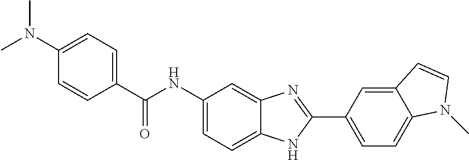 | N-(2-(1-Methyl-1H-indol-5- yl)benzimidazol-5-yl)-4- dimethylaminobenzamide |
| 1735 | 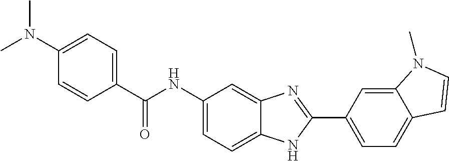 [SMILES: CN(C)c1ccc(C(=O)Nc2ccc3[nH]c(-c4ccc5ccn(C)c5c4)nc3c2)cc1] | N-(2-(1-Methyl-1H-indol-6- yl)benzimidazol-5-yl)-4- dimethylaminobenzamide |
| 1736 | 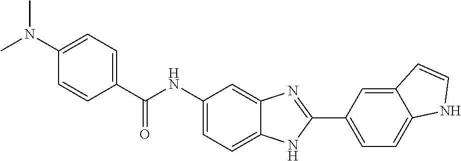 [SMILES: CN(C)c1ccc(C(=O)Nc2ccc3[nH]c(-c4ccc5[nH]ccc5c4)nc3c2)cc1] | N-(2-(1H-Indol-5- yl)benzimidazol-5-yl)-4- dimethylaminobenzamide |
| 1737 | 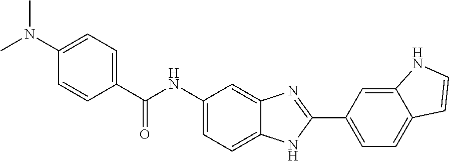 [SMILES: CN(C)c1ccc(C(=O)Nc2ccc3[nH]c(-c4ccc5cc[nH]c5c4)nc3c2)cc1] | N-(2-(1H-Indol-6- yl)benzimidazol-5-yl)-4- dimethylaminobenzamide |
| 1738 | 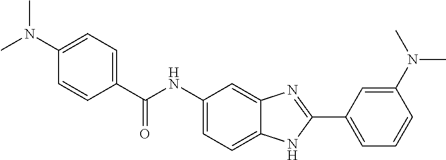 | 4-Dimethylamino-N-(2-(3- dimethylaminophenyl)-1H- benzimidazol-5-yl)benzamide |
| 1739 | 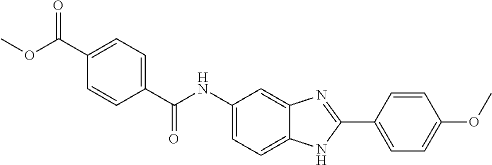 | Methyl 4-(2-(4- methoxyphenyl)-1H- benzimidazol-5-ylcarbamoyl)- benzoate |
| 1740 | 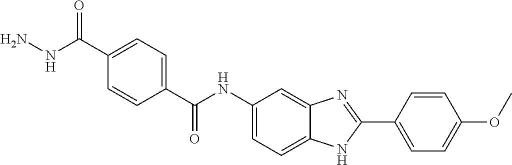 [SMILES: COc1ccc(-c2nc3cc(NC(=O)c4ccc(C(=O)NN)cc4)ccc3[nH]2)cc1] | 4-(2-(4-Methoxyphenyl)-1H- benzimidazol-5-ylcarbamoyl)- benzohydrazide |
| 1741 | 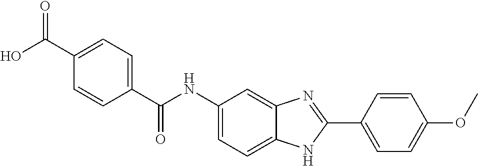 [SMILES: COc1ccc(-c2nc3cc(NC(=O)c4ccc(C(=O)O)cc4)ccc3[nH]2)cc1] | 4-(2-(4-Methoxyphenyl)-1H- benzimidazol-5-ylcarbamoyl)- benzoic acid |
| 1742 | 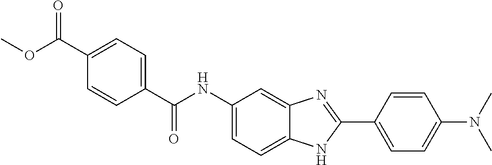 | Methyl 4-(2-(4- dimethylaminophenyl)-1H- benzimidazol-5-ylcarbamoyl)- benzoate |
| 1743 | 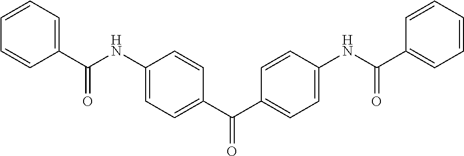 [SMILES: O=C(Nc1ccc(C(=O)c2ccc(NC(=O)c3ccccc3)cc2)cc1)c1ccccc1] | N,N′-(4,4′-Carbonylbis(4,1- phenylene))dibenzamide |
| 1744 | 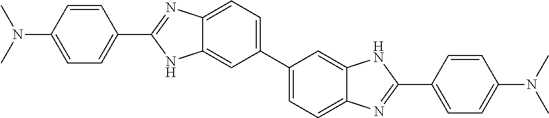 | 4,4′-(5,5′-Bibenzimidazole-2,2′- diyl)bis(N,N-dimethylaniline) |
| 1745 | 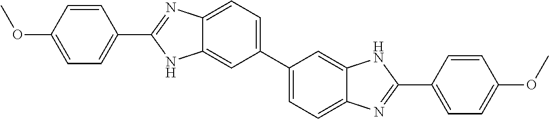 | 4,4′-(5,5′-Bibenzimidazole-2,2′- diyl)bis(anisole) |
| 1746 | 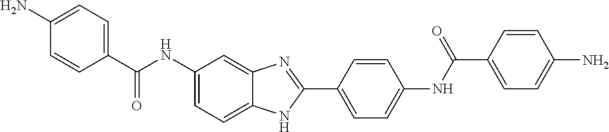 [SMILES: Nc1ccc(C(=O)Nc2ccc(-c3nc4cc(NC(=O)c5ccc(N)cc5)ccc4[nH]3)cc2)cc1] | 4-Amino-N-(4-(5-(4- aminobenzamido)-1H- benzimidazol-2- yl)phenyl)benzamide |
| 1747 | 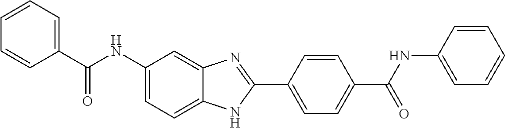 [SMILES: O=C(Nc1ccccc1)c1ccc(-c2nc3cc(NC(=O)c4ccccc4)ccc3[nH]2)cc1] | N-Phenyl-4-(5-benzamido-1H- benzimidazol-2-yl)benzamide |

### Certain Pharmaceutical Agents

In certain embodiments, at least one compound disclosed herein, or pharmaceutically acceptable salt, ester, amide, and/or prodrug thereof, either alone or combined with one or more pharmaceutically acceptable carriers, forms a pharmaceutical agent. Techniques for formulation and administration of compounds of the present embodiments may be found for example, in “Remington's Pharmaceutical Sciences,” Mack Publishing Co., Easton, Pa., 18th edition, 1990, which is incorporated herein by reference in its entirety.

In certain embodiments, a pharmaceutical agent comprising one or more compounds of the present embodiments is prepared using known techniques, including, but not limited to mixing, dissolving, granulating, dragee-making, levigating, emulsifying, encapsulating, entrapping or tabletting processes.

In certain embodiments, a pharmaceutical agent comprising one or more compounds of the present embodiments is a liquid (e.g., a suspension, elixir and/or solution). In certain of such embodiments, a liquid pharmaceutical agent comprising one or more compounds of the present embodiments is prepared using ingredients known in the art, including, but not limited to, water, glycols, oils, alcohols, flavoring agents, preservatives, and coloring agents.

In certain embodiments, a pharmaceutical agent comprising one or more compounds of the present embodiments is a solid (e.g., a powder, tablet, and/or capsule). In certain of such embodiments, a solid pharmaceutical agent comprising one or more compounds of the present embodiments is prepared using ingredients known in the art, including, but not limited to, starches, sugars, diluents, granulating agents, lubricants, binders, and disintegrating agents.

In certain embodiments, a pharmaceutical agent comprising one or more compounds of the present embodiments is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical agent comprising one or more compounds of the present embodiments comprises a delivery system. Examples of delivery systems include, but are not limited to, liposomes and emulsions. Certain delivery systems are useful for preparing certain pharmaceutical agents including those comprising hydrophobic compounds. In certain embodiments, certain organic solvents such as dimethylsulfoxide are used.

In certain embodiments, a pharmaceutical agent comprising one or more compounds of the present embodiments comprises one or more tissue-specific delivery molecules designed to deliver the pharmaceutical agent to specific tissues or cell types. For example, in certain embodiments, pharmaceutical agents include liposomes coated with a tissue-specific antibody.

In certain embodiments, a pharmaceutical agent comprising one or more compounds of the present embodiments comprises a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™, and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, a pharmaceutical agent comprising one or more compounds of the present embodiments comprises a sustained-release system. A non-limiting example of such a sustained-release system is a semi-permeable matrix of solid hydrophobic polymers. In certain embodiments, sustained-release systems may, depending on their chemical nature, release compounds over a period of hours, days, weeks or months.

Certain compounds used in pharmaceutical agent of the present embodiments may be provided as pharmaceutically acceptable salts with pharmaceutically compatible counterions. Pharmaceutically compatible salts may be formed with many acids, including but not limited to hydrochloric, sulfuric, acetic, lactic, tartaric, malic, succinic, etc.

In certain embodiments, a pharmaceutical agent comprising one or more compounds of the present embodiments comprises an active ingredient in a therapeutically effective amount. In certain embodiments, the therapeutically effective amount is sufficient to prevent, alleviate or ameliorate symptoms of a disease or to prolong the survival of the patient being treated. Determination of a therapeutically effective amount is well within the capability of those skilled in the art.

In certain embodiments, a pharmaceutical agent comprising one or more compounds of the present embodiments is formulated as a prodrug. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, a prodrug is an ester. In certain embodiments, such prodrugs are less water soluble than the corresponding active form. In certain instances, such prodrugs possess superior transmittal across cell membranes, where water solubility is detrimental to mobility. In certain embodiments, the ester in such prodrugs is metabolically hydrolyzed to carboxylic acid. In certain instances the carboxylic acid containing compound is the corresponding active form. In certain embodiments, a prodrug comprises a short peptide (polyaminoacid) bound to an acid group. In certain of such embodiments, the peptide is metabolized to form the corresponding active form.

In certain embodiments, a pharmaceutical agent comprising one or more compounds of the present embodiments is useful for treating a conditions or disorder in a mammalian, and particularly in a human patient. Suitable administration routes include, but are not limited to, oral, rectal, transmucosal, intestinal, enteral, topical, suppository, through inhalation, intrathecal, intraventricular, intraperitoneal, intranasal, intraocular and parenteral (e.g., intravenous, intramuscular, intramedullary, and subcutaneous). In certain embodiments, pharmaceutical intrathecals are administered to achieve local rather than systemic exposures. For example, pharmaceutical agents may be injected directly in the area of desired effect (e.g., in the renal or cardiac area).

In certain embodiments, a pharmaceutical agent comprising one or more compounds of the present embodiments is administered in the form of a dosage unit (e.g., tablet, capsule, bolus, etc.). In certain embodiments, such dosage units comprise a compound disclosed herein in a dose from about 1 μg/kg of body weight to about 50 mg/kg of body weight. In certain embodiments, such dosage units comprise a compound disclosed herein in a dose from about 2 μg/kg of body weight to about 25 mg/kg of body weight. In certain embodiments, such dosage units comprise a compound disclosed herein in a dose from about 10 μg/kg of body weight to about 5 mg/kg of body weight. In certain embodiments, pharmaceutical agents are administered as needed, once per day, twice per day, three times per day, or four or more times per day. It is recognized by those skilled in the art that the particular dose, frequency, and duration of administration depends on a number of factors, including, without limitation, the biological activity desired, the condition of the patient, and tolerance for the pharmaceutical agent.

In certain embodiments, a pharmaceutical agent comprising a compound of the present embodiments is prepared for oral administration. In certain of such embodiments, a pharmaceutical agent is formulated by combining one or more compounds of the present embodiments with one or more pharmaceutically acceptable carriers. Certain of such carriers enable compounds of the present embodiments to be formulated as tablets, pills, dragees, capsules, liquids, gels, syrups, slurries, suspensions and the like, for oral ingestion by a patient. In certain embodiments, pharmaceutical agents for oral use are obtained by mixing one or more compounds of the present embodiments and one or more solid excipient. Suitable excipients include, but are not limited to, fillers, such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). In certain embodiments, such a mixture is optionally ground and auxiliaries are optionally added. In certain embodiments, pharmaceutical agents are formed to obtain tablets or dragee cores. In certain embodiments, disintegrating agents (e.g., cross-linked polyvinyl pyrrolidone, agar, or alginic acid or a salt thereof, such as sodium alginate) are added.

In certain embodiments, dragee cores are provided with coatings. In certain of such embodiments, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to tablets or dragee coatings.

In certain embodiments, pharmaceutical agents for oral administration are push-fit capsules made of gelatin. Certain of such push-fit capsules comprise one or more compounds of the present embodiments in admixture with one or more filler such as lactose, binders such as starches, and/or lubricants such as talc or magnesium stearate and, optionally, stabilizers. In certain embodiments, pharmaceutical agents for oral administration are soft, sealed capsules made of gelatin and a plasticizer, such as glycerol or sorbitol. In certain soft capsules, one or more compounds of the present embodiments are be dissolved or suspended in suitable liquids, such as fatty oils, liquid paraffin, or liquid polyethylene glycols. In addition, stabilizers may be added.

In certain embodiments, pharmaceutical agents are prepared for buccal administration. Certain of such pharmaceutical agents are tablets or lozenges formulated in conventional manner.

In certain embodiments, a pharmaceutical agent is prepared for administration by injection (e.g., intravenous, subcutaneous, intramuscular, etc.). In certain of such embodiments, a pharmaceutical agent comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical agents for injection are presented in unit dosage form, e.g., in ampoules or in multi-dose containers. Certain pharmaceutical agents for injection are suspensions, solutions or emulsions in oily or aqueous vehicles, and may contain formulatory agents such as suspending, stabilizing and/or dispersing agents. Certain solvents suitable for use in pharmaceutical agents for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the compounds to allow for the preparation of highly concentrated solutions.

In certain embodiments, a pharmaceutical agent is prepared for transmucosal administration. In certain of such embodiments penetrants appropriate to the barrier to be permeated are used in the formulation. Such penetrants are generally known in the art.

In certain embodiments, a pharmaceutical agent is prepared for administration by inhalation. Certain of such pharmaceutical agents for inhalation are prepared in the form of an aerosol spray in a pressurized pack or a nebulizer. Certain of such pharmaceutical agents comprise a propellant, e.g., dichlorodifluoromethane, trichlorofluoromethane, dichlorotetrafluoroethane, carbon dioxide or other suitable gas. In certain embodiments using a pressurized aerosol, the dosage unit may be determined with a valve that delivers a metered amount. In certain embodiments, capsules and cartridges for use in an inhaler or insufflator may be formulated. Certain of such formulations comprise a powder mixture of a compound of the present embodiments and a suitable powder base such as lactose or starch.

In certain embodiments, a pharmaceutical agent is prepared for rectal administration, such as a suppositories or retention enema. Certain of such pharmaceutical agents comprise known ingredients, such as cocoa butter and/or other glycerides.

In certain embodiments, a pharmaceutical agent is prepared for topical administration. Certain of such pharmaceutical agents comprise bland moisturizing bases, such as ointments or creams. Exemplary suitable ointment bases include, but are not limited to, petrolatum, petrolatum plus volatile silicones, lanolin and water in oil emulsions such as Eucerin™, available from Beiersdorf (Cincinnati, Ohio). Exemplary suitable cream bases include, but are not limited to, Nivea™ Cream, available from Beiersdorf (Cincinnati, Ohio), cold cream (USP), Purpose Cream™, available from Johnson & Johnson (New Brunswick, N.J.), hydrophilic ointment (USP) and Lubriderm™, available from Pfizer (Morris Plains, N.J.).

In certain embodiments, the formulation, route of administration and dosage for a pharmaceutical agent of the present embodiments can be chosen in view of a particular patient's condition. (See e.g., Fingl et al. 1975, in “The Pharmacological Basis of Therapeutics”, Ch. 1p. 1, which is incorporated herein by reference in its entirety). In certain embodiments, a pharmaceutical agent is administered as a single dose. In certain embodiments, a pharmaceutical agent is administered as a series of two or more doses administered over one or more days.

In certain embodiments, a pharmaceutical agent of the present embodiments is administered to a patient between about 0.1% and 500%, 5% and 200%, 10% and 100%, 15% and 85%, 25% and 75%, or 40% and 60% of an established human dosage. Where no human dosage is established, a suitable human dosage may be inferred from ED₅₀ or ID₅₀ values, or other appropriate values derived from in vitro or in vivo studies.

In certain embodiments, a daily dosage regimen for a patient comprises an oral dose of between 0.1 mg and 2000 mg, 5 mg and 1500 mg, 10 mg and 1000 mg, 20 mg and 500 mg, 30 mg and 200 mg, or 40 mg and 100 mg of a compound of the present embodiments. In certain embodiments, a daily dosage regimen is administered as a single daily dose. In certain embodiments, a daily dosage regimen is administered as two, three, four, or more than four doses.

In certain embodiments, a pharmaceutical agent of the present embodiments is administered by continuous intravenous infusion. In certain of such embodiments, from 0.1 mg to 500 mg of a composition of the present embodiments is administered per day.

In certain embodiments, a pharmaceutical agent of the present embodiments is administered for a period of continuous therapy. For example, a pharmaceutical agent of the present embodiments may be administered over a period of days, weeks, months, or years.

Dosage amount, interval between doses, and duration of treatment may be adjusted to achieve a desired effect. In certain embodiments, dosage amount and interval between doses are adjusted to maintain a desired concentration on compound in a patient. For example, in certain embodiments, dosage amount and interval between doses are adjusted to provide plasma concentration of a compound of the present embodiments at an amount sufficient to achieve a desired effect. In certain of such embodiments the plasma concentration is maintained above the minimal effective concentration (MEC). In certain embodiments, pharmaceutical agents of the present embodiments are administered with a dosage regimen designed to maintain a concentration above the MEC for 10-90% of the time, between 30-90% of the time, or between 50-90% of the time.

In certain embodiments in which a pharmaceutical agent is administered locally, the dosage regimen is adjusted to achieve a desired local concentration of a compound of the present embodiments.

In certain embodiments, a pharmaceutical agent may be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the present embodiments formulated in a compatible pharmaceutical carrier may also be prepared, placed in an appropriate container, and labeled for treatment of an indicated condition.

In certain embodiments, a pharmaceutical agent is in powder form for constitution with a suitable vehicle, e.g., sterile pyrogen-free water, before use.

### Certain Combination Therapies

In certain embodiments, one or more compounds of the present embodiments are co-administered with one or more other pharmaceutical agents. In certain embodiments, such one or more other pharmaceutical agents are designed to treat the same disease or condition as the one or more compounds of the present disclosure. In certain embodiments, such one or more other pharmaceutical agents are designed to treat a different disease or condition as the one or more compounds of the present disclosure. In certain embodiments, such one or more other pharmaceutical agents are designed to treat an undesired effect of one or more compounds of the present disclosure. In certain embodiments, one or more compounds of the present disclosure are co-administered with another pharmaceutical agent to treat an undesired effect of that other pharmaceutical agent. In certain embodiments, one or more compounds of the present disclosure and one or more other pharmaceutical agents are administered at the same time. In certain embodiments, one or more compounds of the present disclosure and one or more other pharmaceutical agents are administered at the different times. In certain embodiments, one or more compounds of the present disclosure and one or more other pharmaceutical agents are prepared together in a single formulation. In certain embodiments, one or more compounds of the present disclosure and one or more other pharmaceutical agents are prepared separately.

In certain embodiments, the compounds disclosed herein are administered as combination therapies with EPO or erythropoiesis stimulating agents. In some embodiments, the erythropoiesis stimulating agent is a synthetic protein produced by recombinant DNA technology in mammalian cells into which the human erythropoietin gene has been introduced, e.g., rHuEPO, Epoetin alfa (marketed as Epogen® and Procrit® and identical to endogenous EPO), Epoetin beta (marketed only in Europe as NeoRecormon®), Darbepoetin Alfa (marketed as Aranesp®), or a methoxy polyethylene glycol-conjugated form of epoetin beta (marketed as Micera®). In certain embodiments, the erythropoiesis stimulating agent is Hematide™ or CNTO-530.

In some embodiments, examples of pharmaceutical agents that are administered with compounds disclosed herein include, but are not limited to, anti-cancer treatments, including, but not limited to, chemotherapy and radiation treatment; corticosteroids, including but not limited to prednisone; immunoglobulins, including, but not limited to intravenous immunoglobulin (IVIg); analgesics (e.g., acetaminophen); anti-inflammatory agents, including, but not limited to non-steroidal anti-inflammatory drugs (e.g., ibuprofen, COX-1 inhibitors, and COX-2, inhibitors); salicylates; antibiotics; antivirals; antifungal agents; antidiabetic agents (e.g., biguanides, glucosidase inhibitors, insulins, sulfonylureas, and thiazolidenediones); adrenergic modifiers; diuretics; hormones (e.g., anabolic steroids, androgen, estrogen, calcitonin, progestin, somatostan, and thyroid hormones); immunomodulators; muscle relaxants; antihistamines; osteoporosis agents (e.g., biphosphonates, calcitonin, and estrogens); prostaglandins, antineoplastic agents; psychotherapeutic agents; sedatives; poison oak or poison sumac products; antibodies; and vaccines.

### Methods of Treatment

In certain embodiments, compounds or pharmaceutical agents disclosed herein are EPO receptor partial agonists or selective EPO receptor partial agonists. In some embodiments, EPO receptor partial agonists or selective EPO receptor partial agonists are administered in a dosing regimen such that the first dose results in a biological activity of EPO receptor that is of the same type as that resulting from the presence of naturally occurring EPO, but which is less than the maximum effect induced by EPO, and at least a second dose is administered, which is substantially the same as the first dose, and which also results in a biological activity of EPO receptor that is of the same type as that resulting from the presence of naturally occurring EPO, but which is less than the maximum effect induced by EPO. For example, in some embodiments the first dose will result in EPO receptor biological activity that is of the same type as that resulting from the presence of naturally occurring EPO, but which is less than 95%, or less than 90%, or less than 80%, or less than 70%, or less than 60%, or less than 50%, or less than 40%, or less than 30%, or less than 20%, or less than 10%, or less than 5% of the maximum biological EPO receptor activity induced by EPO, and the at least a second dose results in substantially the same biological activity of EPO receptor as the first dose.

Some embodiments of the present invention include methods for treating a disorder associated with erythropoiesis in a patient in need of such treatment. Some such methods include administering an initial effective amount of a non-peptidyl small molecule mimetic of EPO to the patient, and administering a second effective amount of the mimetic of EPO to the patient, wherein the initial and the second effective amounts are substantially the same over the period of administration of said mimetic of EPO. Some embodiments include administering a third effective amount of the mimetic of EPO to the patient. Some embodiments include administering one or more subsequent effective amounts of the mimetic of EPO to the patient. In some embodiments, the initial and the second effective amounts are the same. In some embodiments, the initial effective amount and the subsequent effective amounts are the same. In some embodiments, the period of administration is at least daily, at least weekly, or at least monthly.

Some embodiments include methods for treating a disorder associated with erythropoiesis in a patient in need of such treatment. Some such methods include administering an effective amount of a non-peptidyl small molecule mimetic of EPO to the patient, wherein the amount administered to the patient is not titrated over the period of time for administration. In some such methods, at least two successive effective amounts are administered to the patient. In some embodiments, the period of time for administration is daily, weekly, or monthly.

In some of the foregoing methods, the disorder associated with erythropoiesis comprises anemia. In some embodiments, the anemia is associated with chronic kidney disease, cancer, palliative cancer therapy, chemotherapy, anemia of chronic disease, congestive heart failure, rheumatoid arthritis, COPD, chronic inflammatory conditions, or HIV infection.

In some of the foregoing methods, the mimetic of EPO comprises an EPO receptor partial agonist. In some embodiments, the mimetic of EPO activates EPO receptor signaling through the PI3K-GATA1 pathway.

More embodimentns of the present invention include uses of a non-peptidyl small molecule mimetic of EPO for treating a disorder associated with erythropoiesis in a patient in need of such treatment. Some such uses include administering an initial effective amount of the mimetic of EPO to the patient, and administering a second effective amount of the mimetic of EPO to the patient, wherein the initial and the second effective amounts are substantially the same over the period of administration of said mimetic of EPO.

Some embodiments include administering a third effective amount of the mimetic of EPO to the patient. Some embodiments include administering one or more subsequent effective amounts of the mimetic of EPO to the patient. In some embodiments, the initial and the second effective amounts are the same. In some embodiments, the initial effective amount and the subsequent effective amounts are the same. In some embodiments, the period of administration is at least daily, at least weekly, or at least monthly.

More embodiments include uses of a non-peptidyl small molecule mimetic of EPO for treating a disorder associated with erythropoiesis erythropoiesis in a patient in need of such treatment. Some such embodiments include administering an effective amount of the mimetic of EPO to the patient, wherein the amount administered to the patient is not titrated over the period of time for administration.

In some embodiments, at least two successive effective amounts are administered to the patient. In some embodiments, the period of time for administration is daily, weekly, or monthly.

In some embodiments, the disorder associated with erythropoiesis comprises anemia. In some embodiments, the anemia is associated with chronic kidney disease, cancer, palliative cancer therapy, chemotherapy, anemia of chronic disease, congestive heart failure, rheumatoid arthritis, COPD, chronic inflammatory conditions, or HIV infection.

In some of the foregoing uses, the mimetic of EPO comprises an EPO receptor partial agonist. In some of the foregoing uses the mimetic of EPO activates EPO receptor signaling through the PI3K-GATA1 pathway.

In some embodiments, compounds of any of Formulae I to X, or any compound specifically disclosed herein, is a hematopoietic growth factor mimetic, a hematopoietic growth factor receptor agonist, or a hematopoietic growth factor receptor antagonist. In some embodiments, compounds of any of Formulae I to X, or any compound specifically disclosed herein, is an EPO mimetic. In some embodiments, compounds of any of Formulae I to X, or any compound specifically disclosed herein, is a selective EPO receptor agonist. In some embodiments, compounds of any of Formulae I to X, or any compound specifically disclosed herein, is a selective EPO receptor partial agonist. In some embodiments, compounds of any of Formulae I to X, or any compound specifically disclosed herein, is a selective EPO receptor antagonist. In some embodiments, compounds of any of Formulae I to X, or any compound specifically disclosed herein, is a selective EPO receptor binding compound.

In some embodiments, compounds disclosed herein are used in a method of modulating an EPO receptor activity in a cell, said method comprising contacting a cell with a compound of any of Formulae I to X, or any compound specifically disclosed herein.

Some embodiments disclosed herein provide a method for identifying a compound that modulates an EPO receptor activity, comprising contacting a cell that expresses an EPO receptor with a compound of any of Formulae I to X, or any compound specifically disclosed herein, and monitoring an effect of the compound on the cell.

Some embodiments disclosed herein provide a method of treating a patient for a disorder requiring EPO receptor modulation, comprising administering to the patient a therapeutically effective amount of a compound of any of Formulae I to X, or any compound specifically disclosed herein. In some embodiments, EPO receptor modulation is EPO receptor activation. In some embodiments, EPO receptor modulation is EPO receptor partial activation. In some embodiments, EPO receptor modulation is EPO receptor inactivation.

### Indications

Some compounds and compositions provided herein can be used to treat various disorders associated with EPO receptor activity, e.g., anemia. In certain embodiments, the anemia is associated with other disorders. For example, anemia may be associated with chronic kidney disease, cancer, palliative cancer therapy, chemotherapy, or anemia of chronic disease, e.g., congestive heart failure, rheumatoid arthritis, COPD, chronic inflammatory conditions, or HIV infection.

### EXAMPLES

### Example 1

### EPO-Dependent Cell Viability Assay

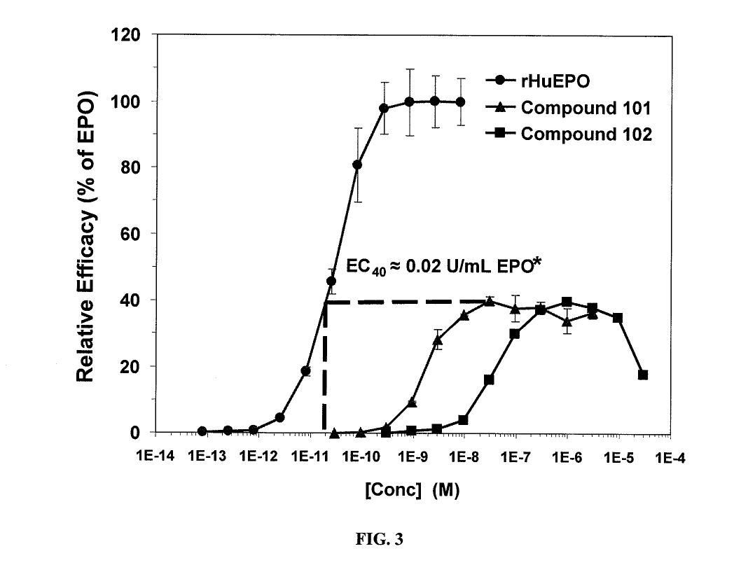

Erythroid cells were treated with various concentrations of recombinant human EPO, Compound 101, or Compound 102, and the viability of treated cells was measured. The maximum efficacies of Compounds 101 and 102 was approximately 40% of the efficacy of EPO, corresponding to EPO EC₄₀ of approximately 0.02 U/ml (FIG. 3). The typical EPO concentration in blood is approximately 0.01 U/ml, with a range from about 0.006 U/ml to about 0.032 U/ml. Compound 101 and Compound 102 exhibited an erythropoietic efficacy that was a fraction of the maximum effect induced by EPO, but greater than would be predicted for EPO at typical serum concentrations.

### Example 2

### Relative Differentiation of Human Bone Marrow Cells

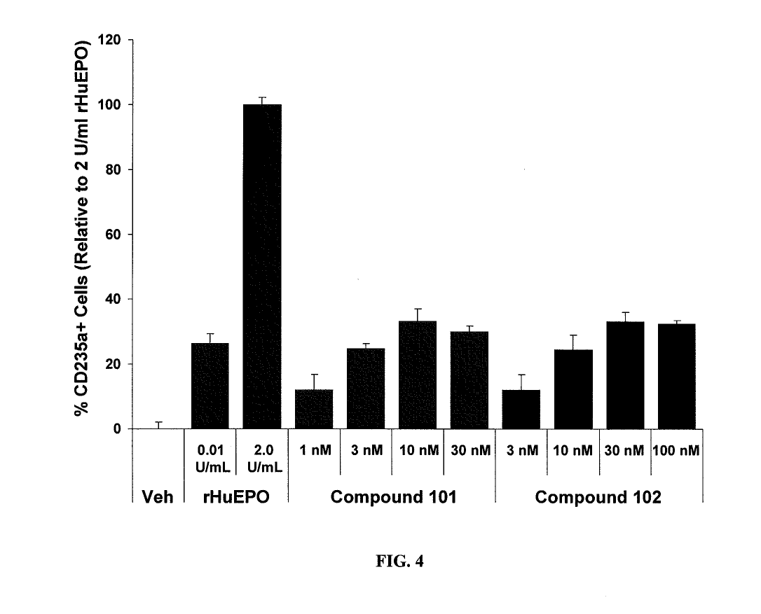

CD34+ human bone marrow cells (BM-HCs) were treated with various concentrations of recombinant human EPO, Compound 101, Compound 102, or control. The number of cells expressing the erythrocyte-specific marker, CD235a (glycophorin A), was measured. Treatment of cells with 10 nm Compound 101 increased the level of expression of CD235a above the level of expression observed in cells treated with 0.01 U/ml EPO (normal serum EPO concentration is about 0.01 U/mL, with a range 0.006-0.032 U/mL) (FIG. 4). The level of expression of CD235a in cells treated with 10 nm Compound 101 was approximately 37% of the level of expression of CD235a in cells treated with 2.0 U/ml EPO.

### Example 3

### Selective Agonists Partially Induce BFU-E Colony Formation in Human CD34+ Bone Marrow Cells

CD34+ human bone marrow cells were incubated with recombinant human EPO, Compound 101, or control. The formation of BFU-E colonies was measured., The results are summarized in Table 1. Compound 101 stimulated the formation of BFU-E colonies with partial efficacy (31%) when incubated with BM-HCs.

TABLE 1

| | | % BFU-E relative |
|---|---|---|
| Treatment | BFU-Es/Dish | to 3 U/ml EPO |
| Vehicle | 1 | 2% |
| rHuEPO (0.01 U/mL) | 7 | 19% |
| rHuEPO (3 U/mL) | 35 | 100% |
| Compound 101 | 11 | 31% |
| (10 nM) | | |

### Example 4

### Activation of EPO receptor-PI3K-GATA1 Signaling

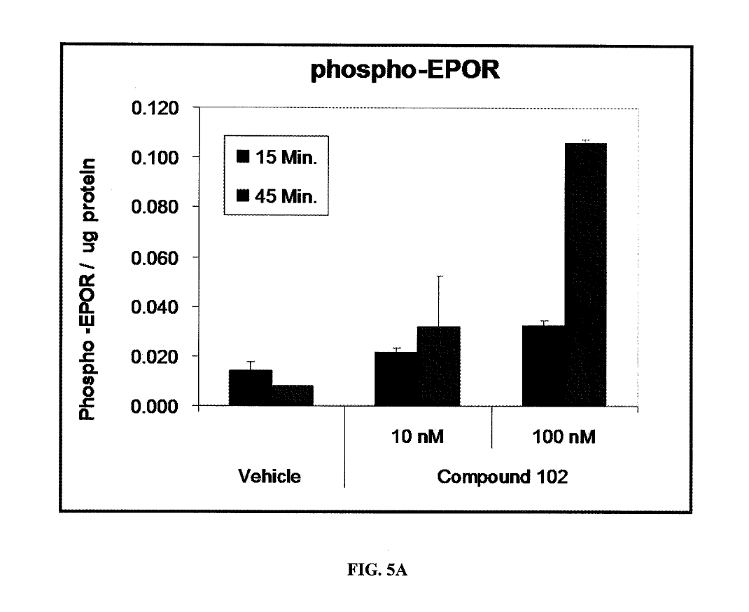

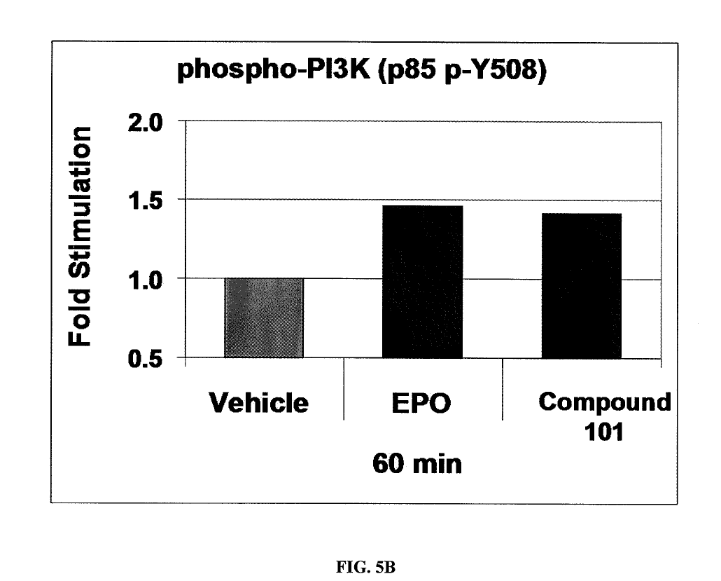

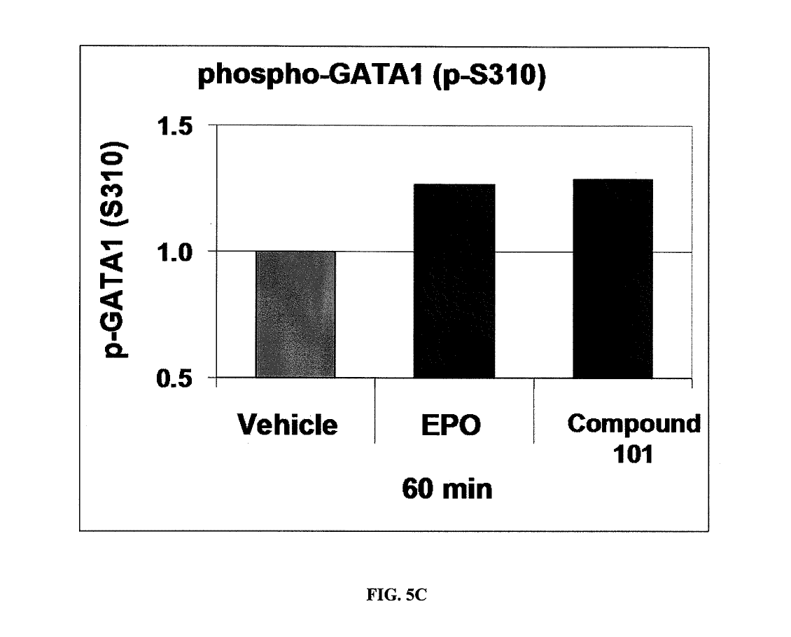

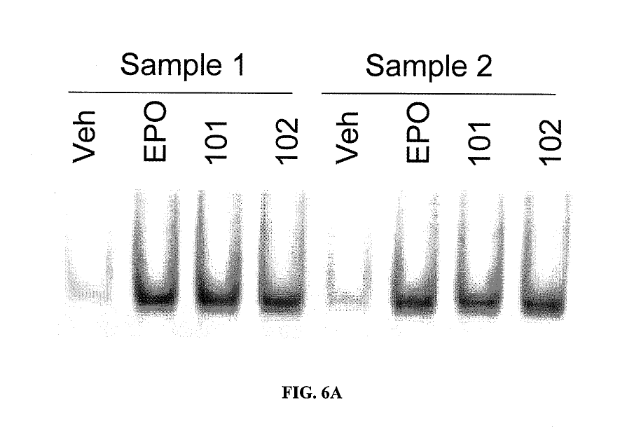

UT7 cells were treated with 10 nM Compound 102, 100 nM Compound 102, or control (vehicle). Phosphorylation of the EPO receptor was measured after 15 minutes or 45 minutes of treatment with Compound 101 or control (vehicle) (FIG. 5A). Phosphorylation of PI3K was measured after 60 minutes of treatment with EPO, Compound 101, or control (vehicle) (FIG. 5B). Phosphorylation of GATA1 was measured after 60 minutes treatment with Compound 102, EPO or control (vehicle) (FIG. 5C). Compound 102 stimulated phosphorylation of at least EPO receptor and GATA1. Compound 101 stimulated phosphorylation of at least PI3K.

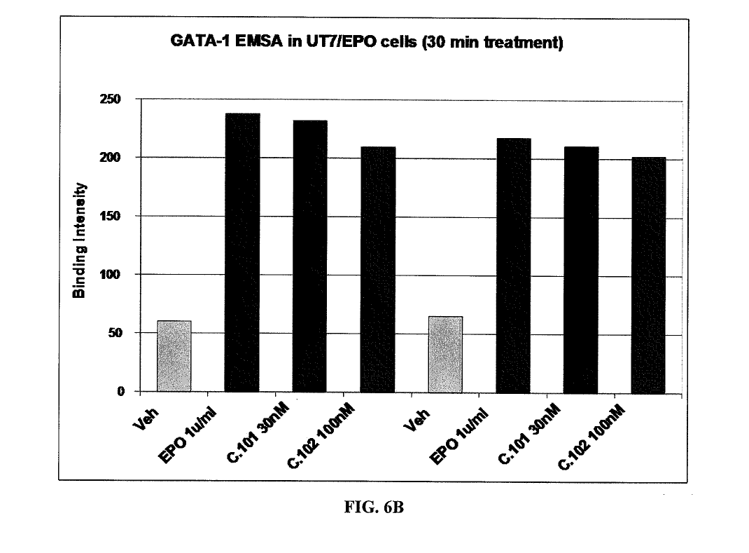

Binding of GATA1 to DNA was measured in an electrophoretic mobility shift assay for GATA1 in UT7 cells after 30 minute treatments with 1 U/ml EPO, 30 nM Compound 102, or 100 nM Compound 101. The result are summarized in FIG. 6A and FIG. 6B. Compounds 101 and 102 induce binding of GATA1 to DNA.

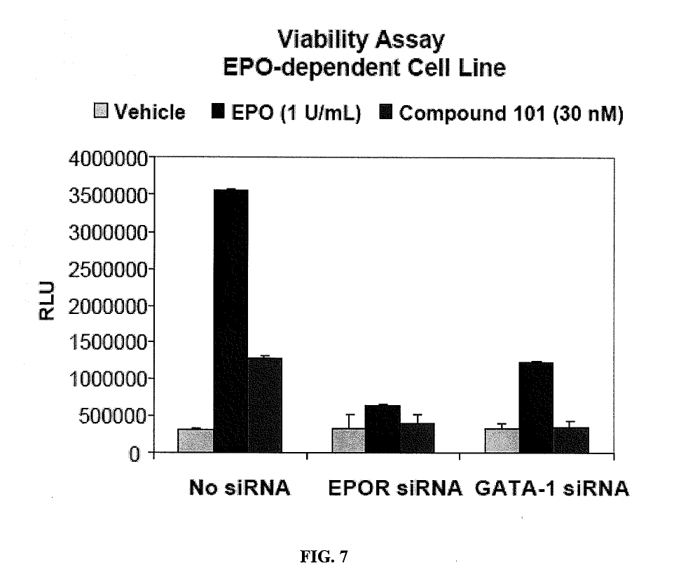

UT7 cells transfected with EPO receptor- and GATA1-specific siRNAs were treated with 1 U/ml EPO, 30 nM Compound 101, or control (vehicle). The relative cell viability of the cells was measured (FIG. 7). Treatment with EPO receptor- and GATA1-specific siRNAs blocks activity of Compound 101.

### Example 5

### CD34+ Cells Treated with Compound 103

In CD34+ human bone marrow hematopoietic cells (BM-HCs), a representative analog, Compound 103, potently (2 nM EC50) increased the percentage of cells positive for the erythrocyte-specific marker CD235a (glycophorin A) with an efficacy partial (42%) to the maximal effect of EPO (3 U/ml), but greater than the efficacy of the normal serum EPO concentration (−0.01 U/ml). The erythropoietic effect of Compound 103 in BM-HCs was additive to the effect of EPO. Compound 103 stimulated the expression of several EPO responsive genes in CD34 positive BM-HCs, including hemoglobin α, EPO receptor, and the anti-apoptotic protein BCL2L1.

In addition, Compound 103 stimulated the formation of BFU-E colonies with partial efficacy (30%) when incubated with BM-HCs for 14 days. The effect of Compound 103 on BM-HCs was specific for the erythroid lineage. Compound 103 did not increase the percentage of BM-HCs positive for the megakaryocyte marker CD41 or the granulocyte marker CD15.

UT7EPO Cells Treated with Compound 103

Using human cell lines, the action of Compound 103 was determined to be dependent on the EPO receptor and involves the selective activation of the PI3K/AKT-GATA1 signaling pathway. In the human EPO-dependent cell line UT7EPO, Compound 103 blocked apoptosis induced by EPO withdrawal (10 nM EC50), and stimulated the expression of BCL2L1 with an efficacy comparable to EPO. Compound 103 stimulated the phosphorylation of EPOR, PI3K, and GATA1, and induced the binding of GATA1 to DNA. Incubation of UT7EPO cells with the PI3K inhibitor LY294002 blocked the effect of Compound 103 on cell survival. However, Compound 103 did not stimulate phosphorylation of STAT5 or ERK/MAPK, or induce STAT5 DNA binding. Transfection of UT7EPO cells with EPO receptor- and GATA1-specific siRNAs blocked the activity of Compound 103.

Mo7e Cells Treated with Compound 103

Compound 103 did not block apoptosis or stimulate BCL2L1 expression in the GM-CSF- and TPO-responsive human leukemia Mo7e cells that lack EPO receptor.

The foregoing data demonstrate that Compound 103 is a novel small molecule selective EPO receptor agonist that unlike erythropoiesis-stimulating agents, selectively activates the EPO receptor/PI3K/GATA1 signal transduction pathway resulting in survival and differentiation of BM-HCs into erythrocytes, possibly through uniquely altering the conformation of the homodimeric EPO receptor. The selective agonists display an efficacy partial to the maximal effect induced by EPO, and lack excessive erythropoietic stimulation that may possibly contribute to the adverse effects of erythropoiesis-stimulating agents. Based on the novel profile of the series, several lead compounds that increase the percentage of CD235a positive BM-HCs with nanomolar potency, and display oral bioavailability in the rat and monkey, have been identified as potential preclinical development candidates.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material. The term “comprising” as used herein is synonymous with “including,” “containing,” or “characterized by,” and is inclusive or open-ended and does not exclude additional, unrecited elements or method steps. All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. A method of treating a disorder associated with erythropoiesis in a patient in need of such treatment, said method comprising:
administering an initial effective amount of a non-peptidyl small molecule mimetic of EPO to the patient; and
administering a second effective amount of the mimetic of EPO to the patient,
wherein the initial and the second effective amounts are substantially the same.

25. The method of claim 24, further comprising administering a third effective amount of the mimetic of EPO to the patient that is substantially the same as the initial and second effective amounts.

26. The method of claim 25, further comprising administering one or more subsequent effective amounts of the mimetic of EPO to the patient that is substantially the same as the initial, second, and third effective amounts.

27. The method of claim 24, wherein the initial and the second effective amounts are the same.

28. The method of claim 24, comprising administering a series of effective amounts that are substantially the same to the patient at least daily, at least weekly, or at least monthly.

29. A method of treating a disorder associated with erythropoiesis to a patient in need of such treatment, said method comprising:
administering an effective amount of a non-peptidyl small molecule mimetic of EPO to the patient, wherein the amount administered to the patient is not titrated over the period of time for administration.

30. The method of claim 29, wherein one or more subsequent effective amounts of the mimetic of EPO are administered to said patient.

31. The method of claim 29, wherein the period of time for administration includes administration daily, weekly, or monthly.

32. The method of claim 24, wherein the disorder associated with erythropoiesis comprises anemia.

33. The method of claim 32, wherein the anemia is associated with chronic kidney disease, cancer, palliative cancer therapy, chemotherapy, anemia of chronic disease, congestive heart failure, rheumatoid arthritis, COPD, chronic inflammatory conditions, or HIV infection.

34. The method of claim 24, wherein the mimetic of EPO is an EPO receptor partial agonist.

35. The method of claim 24, wherein the mimetic of EPO activates EPO receptor signaling through the PI3K-GATA1 pathway.

36. The method of claim 24, wherein the mimetic of EPO comprises a compound of Formula (IX) having the structure: 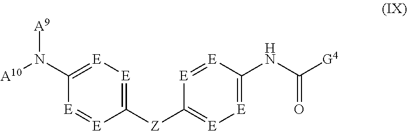
or a pharmaceutically acceptable salt thereof;
wherein:
Z is 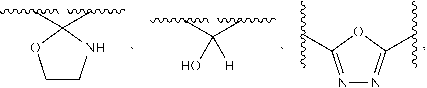
or —C(=E³)-;
each E is separately selected from the group consisting of —CR^{10a}— and N (nitrogen);
each R^{10a} is separately selected from the group consisting of H (hydrogen), halogen, C₁-C₆ alkyl optionally substituted with up to five fluoro, and C₁-C₆ alkoxy optionally substituted with up to five fluoro;
E³ is O (oxygen), N—NHR^{Q} or N—OR^{Q} where R^{Q} in the definition of E³ is selected from the group consisting of hydrogen, C₁-C₆ alkyl, C₂-C₆ alkenyl, —(CH₂)ₘR^{RA}, and —C(═O)(CH₂)ₘR^{RA};
R^{RA} is selected from the group consisting of C₁-C₆ alkyl, aryl, and heteroaryl;
A⁹ is hydrogen or C₁-C₆ alkyl;
A¹⁰ is selected from the group consisting of C₁-C₆ alkyl, C₂-C₆ alkenyl, —C(═O)R^{A}, —C(═O)C(═O)R^{A}, —(CH₂)R^{B}, —(CH₂)OR^{B};
R^{A} is selected from the group consisting of C₁-C₆ alkyl, C₃-C₇ cycloalkyl, heterocycle, polycyclic heterocyclyl, aryl and heteroaryl, each optionally substituted with one or more substituents selected from the group consisting of R¹, R², and R³;
R^{B} is selected from the group consisting of hydrogen, C₁-C₆ alkyl, C₁-C₆ alkoxy, C₂-C₆ alkenyl, C₃-C₇ cycloalkyl, and heteroaryl;
G⁴ is selected from the group consisting of polycyclic heterocyclyl, aryl, and heteroaryl, each optionally substituted with one or more substituents selected from the group consisting of R⁴, R⁵, and R⁶;
each R¹ is separately selected from the group consisting of halogen, cyano, C₁-C₆ heteroalkyl, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, optionally substituted C₃-C₇ cycloalkenyl, an optionally substituted C₁-C₆ haloalkyl, an optionally substituted aryl, and an optionally substituted heteroaryl;
each R² is separately selected from the group consisting of halogen, —O(CH₂)ₘOR^{I}, —(CH₂)ₘOR^{I}, —NR^{J}R^{K}, —(CH₂)ₘSR^{I}, —C(═O)R^{L}, —(CH₂)ₘR^{L}, C₁-C₆ heteroalkyl, an optionally substituted C₁-C₆ alkyl, an optionally substituted C₁-C₆ alkoxy, an optionally substituted C₂-C₆ alkenyl, an optionally substituted C₁-C₆ haloalkyl, and an optionally substituted C₃-C₇ cycloalkyl where said C₃-C₇ cycloalkyl is further optionally fused with aryl or heteroaryl;
each R³ is separately selected from the group consisting of halogen, —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, —(CH₂)ₘS(O)₀₋₂R^{M}, —(CH₂)ₘNHS(O)₀₋₂R^{M}, —(CH₂)ₘCN, —(CH₂)ₘR^{P}, C₁-C₆ alkyl C₁-C₆ alkoxy, C₂-C₆ alkenyl, C₂-C₆ alkynyl, C₃-C₇ cycloalkyl, C₁-C₆ haloalkyl, heterocycle, aryl, polycyclic heterocyclyl, and heteroaryl, said heterocycle, aryl polycyclic heterocyclyl, and heteroaryl in the definition of R³ are each optionally substituted with halogen, hydroxy, cyano, C₁-C₆ alkyl, C₁-C₆ haloalkyl, C₁-C₆ alkoxy, —C(═O)OR^{M}, or —NR^{J}R^{K};
each R⁴ is separately selected from the group consisting of halogen, cyano, C₁-C₆ heteroalkyl, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted C₁-C₆ haloalkyl, an optionally substituted aryl, and an optionally substituted heteroaryl;
each R⁵ is separately selected from the group consisting of halogen, —O(CH₂)ₘOR^{I}, —(CH₂)ₘOR^{I}, —NR^{J}R^{K}, —(CH₂)ₘSR^{I}, —(CH₂)ₘC(═O)R^{L}, —(CH₂)ₘR^{L}, C₁-C₆ heteroalkyl, an optionally substituted C₁-C₆ alkyl, an optionally substituted C₁-C₆ alkoxy, an optionally substituted C₂-C₆ alkenyl, an optionally substituted C₃-C₇ cycloalkyl, and an optionally substituted C₁-C₆ haloalkyl;
each R⁶ is separately selected from the group consisting of halogen, —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, —(CH₂)ₘS(O)₀₋₂R^{M}, —(CH₂)ₘNHS(O)₀₋₂R^{M}, —(CH₂)ₘCN, —(CH₂)ₘR^{P}, C₁-C₆ alkyl, C₁-C₆ alkoxy, C₂-C₆ alkenyl, C₂-C₆ alkynyl, C₃-C₇ cycloalkyl, C₁-C₆ haloalkyl, C₁-C₆ heteroalkyl, heterocycle, aryl, polycyclic heterocyclyl, and heteroaryl, said heterocycle, aryl, polycyclic heterocyclyl, and heteroaryl in the definition of R⁶ are each optionally substituted with halogen, cyano, C₁-C₆ alkyl, C₁-C₆ haloalkyl, C₁-C₆ alkoxy, —C(═O)OR^{M}, or —NR^{J}R^{K};
R^{G} is selected from the group consisting of C₁-C₆ alkyl, C₃-C₆ cycloalkyl, C₃-C₈ cycloalkenyl, C₁-C₆ heteroalkyl, C₁-C₆ heteroalkenyl, C₁-C₆ heteroalkynyl, heterocycle, aryl, and heteroaryl, each optionally substituted with one or more substituents selected from the group consisting of R⁴, R⁵, and R⁶, said aryl and heteroaryl in the definition of R^{G} are each further optionally fused with an optionally substituted nonaromatic heterocycle or an optionally substituted nonaromatic carbocycle, or R^{G} is —OR^{L} or —NR^{P}R^{L};
each R^{I} is separately selected from the group consisting of hydrogen, C₁-C₆ alkyl, C₂-C₄ alkenyl, C₂-C₄ alkynyl, C₃-C₇ cycloalkyl, C₁-C₆ haloalkyl, C₁-C₆ heteroalkyl, and C₁-C₆ heterohaloalkyl;
each —NR^{J}R^{K} is separately selected, wherein R^{J} and R^{K} are each independently selected from the group consisting of hydrogen, C₁-C₆ alkyl optionally substituted with up to 5 fluoro, —(CH₂)ₘOR^{JA}, —(CH₂)ₘNR^{JB}R^{JC}, —(CH₂)ₘR^{R}, C₃-C₇ cycloalkyl, heterocycle, aryl and heteroaryl, said C₃-C₇ cycloalkyl, heterocycle, aryl and heteroaryl in the definition of R^{J} and R^{K} are each independently optionally substituted with one or more substituents selected from the group consisting of halo, C₁-C₆ alkyl, C₁-C₆ haloalkyl, C₁-C₆ alkoxy, aryl and heteroaryl, said aryl and heteroaryl substituent off of R^{J} and R^{K} are each optionally substituted with one or more halo, C₁-C₆ alkyl, C₁-C₆ haloalkyl, C₁-C₆ alkoxy, or —(CH₂)ₘNR^{KA}R^{KB}; or —NR^{J}R^{K} is an optionally substituted non-aromatic heterocycle linked through a ring nitrogen atom;
each R^{JA} is independently selected from the group consisting of hydrogen, C₁-C₆ alkyl, and C₁-C₆ haloalkyl;
each —NR^{JB}R^{JC} is separately selected, wherein R^{JB} and R^{JC} are each independently selected from the group consisting of hydrogen, C₁-C₆ alkyl, and C₁-C₆ haloalkyl;
each —NR^{KA}R^{KB} is separately selected, wherein R^{KA} and R^{KB} are each independently selected from the group consisting of hydrogen, C₁-C₆ alkyl, and C₁-C₆ haloalkyl;
each R^{M} is independently selected from the group consisting of hydrogen, an optionally substituted C₁-C₆ alkyl, an optionally substituted C₂-C₄ alkenyl, an optionally substituted C₂-C₄ alkynyl, an optionally substituted C₃-C₇ cycloalkyl, an optionally substituted C₃-C₇ cycloalkenyl, and —(CH₂)ₘR^{P};
each —NR^{N}R^{O} is separately selected, wherein R^{N} and R^{O} are each independently selected from the group consisting of hydrogen, —(CH₂)ₘNR^{NA}R^{NB}, aryl and heteroaryl, said aryl and heteroaryl in the definition of R^{N} and R^{O} are each independently optionally substituted with one or more substituents selected from the group consisting of —(CH₂)ₘNR^{OA}R^{OB}, halo, C₁-C₆ alkyl, C₁-C₆ haloalkyl, C₁-C₆ alkoxy, aryl and heteroaryl, said aryl and heteroaryl substituent off of R^{N} and R^{O} are each optionally substituted with one or more halo, C₁-C₆ alkyl, C₁-C₆ haloalkyl, C₁-C₆ alkoxy, or —NR^{NA}R^{NB} each —NR^{NA}R^{NB} is separately selected, wherein R^{NA} and R^{NB} are each independently selected from the group consisting of hydrogen, C₁-C₆ alkyl, and C₁-C₆ haloalkyl;
each —NR^{A}R^{B} is separately selected, wherein R^{OA} and R^{OB} are each independently selected from the group consisting of hydrogen, C₁-C₆ alkyl, and C₁-C₆ haloalkyl;
R^{P} is selected from the group consisting of hydrogen and C₁-C₆ alkyl;
each R^{L} is independently selected from the group consisting of C₃-C₇ cycloalkyl, optionally substituted C₁-C₆ alkyl, optionally substituted C₁-C₆ alkoxy, —(CH₂)ₘOR^{LA}, —(CH₂)ₘNR^{LB}R^{LC}, aryl and heteroaryl, said aryl and heteroaryl in the definition of R^{L} are each independently optionally substituted with one or more substituents selected from the group consisting of halo, C₁-C₆ alkyl, C₁-C₆ haloalkyl, C₁-C₆ alkoxy, —(CH₂)ₘNR^{LD}R^{LE}, aryl and heteroaryl, said aryl and heteroaryl substituent off of R^{L} are each optionally substituted with one or more halo, C₁-C₆ alkyl, C₁-C₆ haloalkyl, C₁-C₆ alkoxy, or —(CH₂)ₘNR^{LF}R^{LG};
each R^{LA} is independently selected from the group consisting of hydrogen, C₁-C₆ alkyl, and C₁-C₆ haloalkyl;
R^{LB} and R^{LC} are each independently selected from the group consisting of hydrogen, C₁-C₆ alkyl, C₁-C₆ haloalkyl, and C₁-C₆ heteroalkenyl, said C₁-C₆ alkyl, C₁-C₆ haloalkyl, and C₁-C₆ heteroalkenyl each optionally substituted with one or more halogen, cyano, or —(CH₂)ₘC(═O)OH; or —NR^{LB}R^{LC} is an optionally substituted non-aromatic heterocycle linked through a ring nitrogen atom;
each —NR^{LD}R^{LE} is separately selected, wherein R^{LD} and R^{LE} are each independently selected from the group consisting of hydrogen, aryl, heteroaryl, and optionally substituted C₁-C₆ alkyl, said aryl and heteroaryl in the definition of R^{LD} and R^{LE} are each optionally substituted with C₁-C₆ alkyl or C₁-C₆ alkoxy; or —NR^{LD}R^{LE} is an optionally substituted non-aromatic heterocycle linked through a ring nitrogen atom;
each —NR^{LF}R^{LG} is separately selected, wherein R^{LF} and R^{LG} are each independently selected from the group consisting of hydrogen, and C₁-C₆ alkyl; or —NR^{LF}R^{LG} is an optionally substituted non-aromatic heterocycle linked through a ring nitrogen atom;
R^{R} is selected from the group consisting of C₁-C₆ alkyl, an optionally substituted aryl, and an optionally substituted heteroaryl; and
each m is independently 0, 1, 2, or 3.

37. The method of claim 24, wherein the EPO mimetic comprises a compound of Formula (I) having the structure: 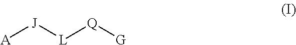
or a pharmaceutically acceptable salt, ester, stereoisomer, tautomer, or prodrug thereof;
wherein:
A-J is 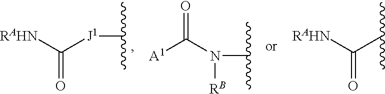
and Q-G is 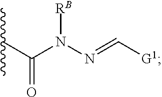
or A-J is 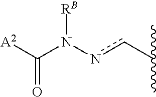
and Q-G is 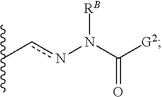
or A-J is 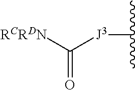
and Q-G is 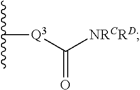
or A-J is 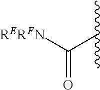
and Q-G is 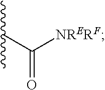
or A-J is 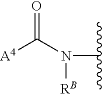
and Q-G is 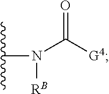
or A-J is 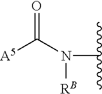
and Q-G is 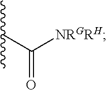
A¹ is selected from the group consisting of C₃-C₇ cycloalkenyl, C₃-C₇ cycloalkyl, C₁-C₆ alkyl, C₂-C₆ alkenyl, C₁-C₆ alkoxy, —(CH₂)ₘNR^{P}R^{L}, heterocycle, aryl, and heteroaryl, said C₃-C₇ cycloalkenyl, C₃-C₇ cycloalkyl, heterocycle, aryl, and heteroaryl, each optionally substituted with one or more substituents selected from the group consisting of R¹, R², and R³, said aryl and heteroaryl in the definition of A¹ are each further optionally fused with an optionally substituted nonaromatic heterocycle or an optionally substituted nonaromatic carbocycle;
J¹ is selected from the group consisting of —(CH₂)ᵣNR^{B}C(═O)(CH₂)ₘ— and —(CH₂)ᵣNR^{B}(CH₂)ₘ—, and —(CH₂)ᵣ—;
G¹ is selected from the group consisting of aryl and heteroaryl, each optionally substituted with one or more substituents selected from the group consisting of R⁴, R⁵, and R⁶, said aryl and heteroaryl in the definition of G¹ are each further optionally fused with an optionally substituted nonaromatic heterocycle or an optionally substituted nonaromatic carbocycle;
A² is selected from the group consisting of aryl and heteroaryl, each optionally substituted with one or more substituents selected from the group consisting of R¹, R², and R³, said aryl and heteroaryl in the definition of A² are each further optionally fused with an optionally substituted nonaromatic heterocycle or an optionally substituted nonaromatic carbocycle;
G² is selected from the group consisting of aryl and heteroaryl, each optionally substituted with one or more substituents selected from the group consisting of R⁴, R⁵, and R⁶, said aryl and heteroaryl in the definition of G² are each further optionally fused with an optionally substituted nonaromatic heterocycle or an optionally substituted nonaromatic carbocycle;
J³ is selected from the group consisting of an optionally substituted aryl, —(CH₂)ₘNR^{B}C(═O)(CH₂)ₘ—, —(CH₂)ᵣO(CH₂)ₘ—, —(CH₂)ᵣNR^{B}(CH₂)ₘ—, and —(CH═CH)ₘ—;
Q³ is selected from the group consisting of an optionally substituted aryl, —(CH₂)ᵣNR^{B}C(═O)(CH₂)ₘ—, —(CH₂)ᵣO(CH₂)ₘ—, —(CH₂)ᵣNR^{B}(CH₂)ₘ—, and —(CH═CH)ᵣ—;
A⁴ is selected from the group consisting of C₃-C₇ cycloalkenyl, C₃-C₇ cycloalkyl, C₁-C₆ alkyl, C₁-C₆ heteroalkyl, C₂-C₆ alkenyl, C₁-C₆ alkoxy, —(CH₂)ₘNR^{P}R^{L}, heterocycle, aryl, and heteroaryl, said C₃-C₇ cycloalkenyl, C₃-C₇ cycloalkyl, C₁-C₆ alkyl, C₁-C₆ heteroalkyl, C₂-C₆ alkenyl, heterocycle, aryl, and heteroaryl, each optionally substituted with one or more substituents selected from the group consisting of R¹, R², and R³, and said aryl and heteroaryl in the definition of A⁴ are each further optionally fused with an optionally substituted nonaromatic heterocycle or an optionally substituted nonaromatic carbocycle;
G⁴ is selected from the group consisting of C₃-C₇ cycloalkenyl, aryl, and heteroaryl, each optionally substituted with one or more substituents selected from the group consisting of R⁴, R⁵, and R⁶, said aryl and heteroaryl in the definition of G⁴ are each further optionally fused with an optionally substituted nonaromatic heterocycle or an optionally substituted nonaromatic carbocycle;
A⁵ is selected from the group consisting of aryl and heteroaryl, each optionally substituted with one or more substituents selected from the group consisting of R¹, R², and R³, said aryl and heteroaryl in the definition of A⁵ are each further optionally fused with an optionally substituted nonaromatic heterocycle or an optionally substituted nonaromatic carbocycle;
R^{A} is selected from the group consisting of —(CH₂)ₚR^{L}, —(CH₂)ₚOR^{L}, —SO₂R^{L}, —C(═O)R^{L}, —C(═O)NR^{N}R^{O}, —(CH₂)ₚNR^{N}R^{O}, an aryl and an heteroaryl, said aryl and heteroaryl in the definition of R^{A} are each optionally substituted with halogen, cyano, C₁-C₆ haloalkyl, C₁-C₆ alkyl, C₁-C₆ alkoxy, C₃-C₇ cycloalkyl, C₃-C₇ cycloalkenyl, —C(═O)NR^{N}R^{O}, —OC(═O)NR^{N}R^{O}, —NHC(═O)NR^{N}R^{O}, —O(CH₂)_{q}NR^{N}R^{O}, —NH(CH₂)_{q}NR^{N}R^{O}, —(CH₂)ₚNR^{N}R^{O}, an optionally substituted aryl and an optionally substituted heteroaryl, and said aryl and heteroaryl in the definition of R^{A} are each further optionally fused with an optionally substituted nonaromatic heterocycle or an optionally substituted nonaromatic carbocycle;
each R^{B} is separately selected from the group consisting of hydrogen, an optionally substituted C₁-C₆ alkyl, an optionally substituted C₂-C₆ alkenyl, and an optionally substituted C₃-C₇ cycloalkyl;
each —NR^{C}R^{D} is separately selected, wherein each R^{C} is independently selected from the group consisting of hydrogen and an optionally C₁-C₆ alkyl, and each R^{D} is independently selected from the group consisting of aryl and heteroaryl, said aryl and heteroaryl in the definition of R^{D} are each optionally substituted with halogen, cyano, C₁-C₆ alkyl, C₁-C₆ alkoxy, C₃-C₇ cycloalkyl, C₃-C₇ cycloalkenyl, —C(═O)NR^{N}R^{O}, —OC(═O)NR^{N}R^{O}, —NHC(═O)NR^{N}R^{O}, —O(CH₂)_{q}NR^{N}R^{O}, —NH(CH₂)_{q}NR^{N}R^{O}—(CH₂)ₚNR^{N}R^{O}, an optionally substituted aryl and an optionally substituted heteroaryl, and said aryl and heteroaryl in the definition of R^{D} are each further optionally fused with an optionally substituted nonaromatic heterocycle or an optionally substituted nonaromatic carbocycle;
each —NR^{E}R^{F} is separately selected, wherein each R^{E} is independently selected from the group consisting of hydrogen and an optionally C₁-C₆ alkyl, and each R^{F} is independently selected from the group consisting of aryl and heteroaryl, said aryl and heteroaryl in the definition of R^{F} are each optionally substituted with halogen, cyano, C₁-C₆ alkyl, C₁-C₆ alkoxy, C₃-C₇ cycloalkyl, C₃-C₇ cycloalkenyl, —C(═O)NR^{N}R^{O}, —OC(═O)NR^{N}R^{O}, —NHC(═O)NR^{N}R^{O}, —O(CH₂)_{q}NR^{N}R^{O}, —NH(CH₂)_{q}NR^{N}R^{O}—(CH₂)ₚNR^{N}R^{O}, an optionally substituted aryl and an optionally substituted heteroaryl, and said aryl and heteroaryl in the definition of R^{F} are each further optionally fused with an optionally substituted nonaromatic heterocycle or an optionally substituted nonaromatic carbocycle;
R^{G} within the definition of —NR^{G}R^{H} is selected from the group consisting of C₁-C₆ alkyl, C₃-C₆ cycloalkyl, C₃-C₈ cycloalkenyl, C₁-C₆ heteroalkyl, C₁-C₆ heteroalkenyl, C₁-C₆ heteroalkynyl, heterocycle, aryl, and heteroaryl, each optionally substituted with one or more substituents selected from the group consisting of R⁴, R⁵, and R⁶, said aryl and heteroaryl in the definition of R^{G} are each further optionally fused with an optionally substituted nonaromatic heterocycle or an optionally substituted nonaromatic carbocycle, or R^{G} is —OR^{L} or —NR^{P}R^{L};
R^{H} within the definition of —NR^{G}R^{H} is selected from the group consisting of hydrogen, C₁-C₆ alkyl, C₂-C₆ alkenyl, C₃-C₇ cycloalkyl, and C₁-C₃ haloalkyl, or —NR^{G}R^{H} is an optionally substituted non-aromatic heterocycle linked through a ring nitrogen atom;
each R¹ is separately selected from the group consisting of halogen, cyano, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, optionally substituted C₃-C₇ cycloalkenyl, an optionally substituted C₁-C₆ haloalkyl, an optionally substituted C₁-C₆ heteroalkyl, an optionally substituted aryl, and an optionally substituted heteroaryl;
each R² is separately selected from the group consisting of halogen, —O(CH₂)ₘOR^{I}, —(CH₂)ₘOR^{I}, —NR^{J}R^{K}, —(CH₂)ₘSR^{I}, —C(═O)R^{L}, —(CH₂)ₘR^{L}, an optionally substituted C₁-C₆ alkyl, an optionally substituted C₁-C₆ alkoxy, an optionally substituted C₂-C₆ alkenyl, an optionally substituted C₁-C₆ haloalkyl, an optionally substituted C₁-C₆ heteroalkyl, and an optionally substituted C₃-C₇ cycloalkyl where said C₃-C₇ cycloalkyl is further optionally fused with aryl or heteroaryl;
each R³ is separately selected from the group consisting of halogen, —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, —(CH₂)ₘS(O)₀₋₂R^{M}, —(CH₂)ₘNHS(O)₀₋₂R^{M}, —(CH₂)ₘNO₂, —(CH₂)ₘCN, —(CH₂)ₘR^{P}, C₁-C₆ alkyl C₁-C₆ alkoxy, C₂-C₆ alkenyl, C₂-C₆ alkynyl, C₃-C₇ cycloalkyl, C₁-C₆ haloalkyl, heterocycle, aryl, and heteroaryl, said heterocycle, aryl and heteroaryl in the definition of R³ are each optionally substituted with halogen, cyano, C₁-C₆ alkyl, C₁-C₆ haloalkyl, C₁-C₆ alkoxy, or —NR^{J}R^{K};
each R⁴ is separately selected from the group consisting of halogen, cyano, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted C₁-C₆ haloalkyl, an optionally substituted C₁-C₆ heteroalkyl, an optionally substituted aryl, and an optionally substituted heteroaryl;
each R⁵ is separately selected from the group consisting of halogen, —O(CH₂)ₘOR^{I}, —(CH₂)ₘOR^{I}, —NR^{J}R^{K}, —(CH₂)ₘSR^{I}, —C(═O)R^{L}, —(CH₂)ₘR^{L}, an optionally substituted C₁-C₆ alkyl, an optionally substituted C₁-C₆ alkoxy, an optionally substituted C₂-C₆ alkenyl, an optionally substituted C₃-C₇ cycloalkyl, an optionally substituted C₁-C₆ haloalkyl, and an optionally substituted C₁-C₆ heteroalkyl;
each R⁶ is separately selected from the group consisting of halogen, —(CH₂)ₘOR^{G}, —NR^{L}C(═O)R^{M}, —NR^{L}C(═O)OR^{M}, —NR^{L}C(═O)NR^{N}R^{O}, —NR^{N}R^{O}, —(CH₂)ₘS(O)₀₋₂R^{M}, —(CH₂)ₘNHS(O)₀₋₂R^{M}, —(CH₂)ₘNO₂, —(CH₂)ₘCN, —(CH₂)ₘR^{P}, C₁-C₆ alkyl, C₁-C₆ alkoxy, C₂-C₆ alkenyl, C₂-C₆ alkynyl, C₃-C₇ cycloalkyl, C₁-C₆ haloalkyl, C₁-C₆ heteroalkyl, heterocycle, aryl, and heteroaryl, said heterocycle, aryl and heteroaryl in the definition of R⁶ are each optionally substituted with halogen, cyano, C₁-C₆ alkyl, C₁-C₆ haloalkyl, C₁-C₆ alkoxy, or —NR^{J}R^{K};
each R^{I} is separately selected from the group consisting of hydrogen, C₁-C₆ alkyl, C₂-C₄ alkenyl, C₂-C₄ alkynyl, C₃-C₇ cycloalkyl, C₁-C₆ haloalkyl, C₁-C₆ heteroalkyl, and C₁-C₆ heterohaloalkyl;
each —NR^{J}R^{K} is separately selected, wherein R^{J} and R^{K} are each independently selected from the group consisting of hydrogen, C₁-C₆ alkyl optionally substituted with up to 5 fluoro, —(CH₂)ₘOR^{JA}, —(CH₂)ₘNR^{JB}R^{JC}, —(CH₂)ₘR^{R}, C₃-C₇ cycloalkyl, heterocycle, aryl and heteroaryl, said C₃-C₇ cycloalkyl, heterocycle, aryl and heteroaryl in the definition of R^{J} and R^{K} are each independently optionally substituted with one or more substituents selected from the group consisting of halo, C₁-C₆ alkyl, C₁-C₆ haloalkyl, C₁-C₆ alkoxy, aryl and heteroaryl, said aryl and heteroaryl substituent off of R^{J} and R^{K} are each optionally substituted with one or more halo, C₁-C₆ alkyl, C₁-C₆ haloalkyl, C₁-C₆ alkoxy, or —(CH₂)ₘNR^{KA}R^{K}B; or —NR^{J}R^{K} is an optionally substituted non-aromatic heterocycle linked through a ring nitrogen atom; or —NR^{J}R^{K} is an optionally substituted C₁-C₆ alkylideneamino;
each R^{JA} is independently selected from the group consisting of hydrogen, C₁-C₆ alkyl, and C₁-C₆ haloalkyl;
each —NR^{JB}R^{JC} is separately selected, wherein R^{JB} and R^{JC} are each independently selected from the group consisting of hydrogen, C₁-C₆ alkyl, and C₁-C₆ haloalkyl;
each —NR^{KA}R^{KB} is separately selected, wherein R^{KA} and R^{KB} are each independently selected from the group consisting of hydrogen, C₁-C₆ alkyl, and C₁-C₆ haloalkyl;
each R^{M} is independently selected from the group consisting of hydrogen, an optionally substituted C₁-C₆ alkyl, an optionally substituted C₂-C₄ alkenyl, an optionally substituted C₂-C₄ alkynyl, an optionally substituted C₃-C₇ cycloalkyl, an optionally substituted C₃-C₇ cycloalkenyl, and —(CH₂)ₘR^{P};
each —NR^{N}R^{O} is separately selected, wherein R^{N} and R^{O} are each independently selected from the group consisting of hydrogen, —(CH₂)ₘNR^{NA}R^{NB}, aryl and heteroaryl, said aryl and heteroaryl in the definition of R^{N} and R^{O} are each independently optionally substituted with one or more substituents selected from the group consisting of —(CH₂)ₘNR^{OA}R^{OB}, halo, C₁-C₆ alkyl, C₁-C₆ haloalkyl, C₁-C₆ alkoxy, aryl and heteroaryl, said aryl and heteroaryl substituent off of R^{N} and R^{O} are each optionally substituted with one or more halo, C₁-C₆ alkyl, C₁-C₆ haloalkyl, C₁-C₆ alkoxy, or —NR^{NA}R^{NB} each —NR^{NA}R^{NB} is separately selected, wherein R^{NA} and R^{NB} are each independently selected from the group consisting of hydrogen, C₁-C₆ alkyl, and C₁-C₆ haloalkyl;
each —NR^{A}R^{B} is separately selected, wherein R^{OA} and R^{OB} are each independently selected from the group consisting of hydrogen, C₁-C₆ alkyl, and C₁-C₆ haloalkyl;
R^{P} is selected from the group consisting of hydrogen and C₁-C₆ alkyl;
R^{L} is selected from the group consisting of C₃-C₇ cycloalkyl, optionally substituted C₁-C₆ alkyl, optionally substituted C₁-C₆ alkoxy, —(CH₂)ₘOR^{LA}, —(CH₂)ₘNR^{LB}R^{LC}, aryl and heteroaryl, said aryl and heteroaryl in the definition of R^{L} are each independently optionally substituted with one or more substituents selected from the group consisting of halo, C₁-C₆ alkyl, C₁-C₆ haloalkyl, C₁-C₆ alkoxy, —(CH₂)ₘNR^{LD}R^{LE}, aryl and heteroaryl, said aryl and heteroaryl substituent off of R^{L} are each optionally substituted with one or more halo, C₁-C₆ alkyl, C₁-C₆ haloalkyl, C₁-C₆ alkoxy, or —(CH₂)ₘNR^{LF}R^{LG};
R^{LA} is selected from the group consisting of hydrogen, C₁-C₆ alkyl, and C₁-C₆ haloalkyl;
R^{LB} and R^{LC} are each independently selected from the group consisting of hydrogen, C₁-C₆ alkyl, C₁-C₆ haloalkyl, and C₁-C₆ heteroalkenyl; or —NR^{LB}R^{LC} is an optionally substituted non-aromatic heterocycle linked through a ring nitrogen atom;
each —NR^{LD}R^{LE} is separately selected, wherein R^{LD} and R^{LE} are each independently selected from the group consisting of hydrogen, aryl, heteroaryl, and optionally substituted C₁-C₆ alkyl, said aryl and heteroaryl in the definition of R^{LD} and R^{LE} are each optionally substituted with C₁-C₆ alkyl or C₁-C₆ alkoxy; or —NR^{LD}R^{LE} is an optionally substituted non-aromatic heterocycle linked through a ring nitrogen atom;
each —NR^{LF}R^{LG} is separately selected, wherein R^{LF} and R^{LG} are each independently selected from the group consisting of hydrogen, and C₁-C₆ alkyl; or —NR^{LF}R^{LG} is an optionally substituted non-aromatic heterocycle linked through a ring nitrogen atom;
L is selected from the group consisting of —O(CH₂)ₚO—, C₁-C₇ alkyl, C₁-C₇ heteroalkyl, C₃-C₇ cycloalkyl, C₃-C₇ cycloalkenyl, an optionally substituted heterocycle, an optionally substituted aryl, and an optionally substituted heteroaryl, or L is selected from the group consisting of L¹-L², L¹-O-L², L¹-S-L², L¹-NR⁹-L², L¹-L²-L³, L¹-L²-L³-L⁴, L¹-C(=E)-L², and L¹-CR⁷R⁸-L²;
L¹ is selected from the group consisting of an optionally substituted aryl, an optionally substituted heteroaryl, and an optionally substituted heterocycle;
L² is selected from the group consisting of an optionally substituted aryl, an optionally substituted heteroaryl, and an optionally substituted heterocycle;
L³ is selected from the group consisting of an optionally substituted aryl, an optionally substituted heteroaryl, and an optionally substituted heterocycle;
L⁴ is an optionally substituted aryl;
E is O (oxygen), N—NHR^{Q} or N—OR^{Q} where R^{Q} in the definition of E is selected from the group consisting of hydrogen, an optionally substituted C₁-C₆ alkyl, an optionally substituted C₂-C₆ alkenyl, —(CH₂)ₘR^{R}, and —C(═O)(CH₂)ₘR^{R};
R^{R} is selected from the group consisting of C₁-C₆ alkyl, an optionally substituted aryl, and an optionally substituted heteroaryl;
R⁷ and R⁸ are each independently selected from the group consisting of hydrogen, C₁-C₆ alkyl, C₁-C₆ heteroalkyl, and —OH; or CR⁷R⁸ is a three- to eight-membered optionally substituted carbocycle, which optionally has one to three additional hetero atoms incorporated in the ring;
R⁹ is selected from the group consisting of hydrogen, C₁-C₆ alkyl, C₁-C₆ haloalkyl, C₁-C₆ heteroalkyl, C₃-C₇cycloalkylC(O)— and C₁-C₆ alkylC(O)—;
each m is independently 0, 1, 2, or 3;
each p is independently 0, 1, 2, 3, 4, 5, or 6;
each q is independently 1, 2, 3, 4, 5, or 6;
each r is independently 1, 2, 3, or 4; and
any bond represented by a dashed and solid line represents a bond selected from the group consisting of a single bond and a double bond.

38. The method of claim 24, wherein the mimetic of EPO comprises a compound of Formula (II) having the structure: 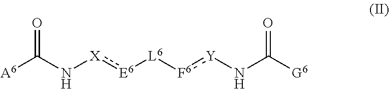
or a pharmaceutically acceptable salt, ester, stereoisomer, tautomer, or prodrug thereof;
wherein:
A⁶ is selected from the group consisting of aryl and heteroaryl, each optionally substituted with one or more substituents selected from the group consisting of R¹, R¹², and R¹³, said aryl and heteroaryl in the definition of A⁶ are each further optionally fused with an optionally substituted nonaromatic heterocycle or an optionally substituted nonaromatic carbocycle;
G⁶ is selected from the group consisting of aryl and heteroaryl, each optionally substituted with one or more substituents selected from the group consisting of R¹⁴, R¹⁵, and R¹⁶, said aryl and heteroaryl in the definition of G⁶ are each further optionally fused with an optionally substituted nonaromatic heterocycle or an optionally substituted nonaromatic carbocycle;
L⁶ is an optionally substituted aryl, or an optionally substituted heteroaryl; where the aryl and heteroaryl in the definition of L⁶ are optionally fused with a nonaromatic heterocycle or a nonaromatic carbocycle; or L⁶ is selected from the group consisting of an optionally substituted 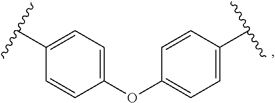 [SMILES: *c1ccc(Oc2ccc(*)cc2)cc1]
and an optionally substituted 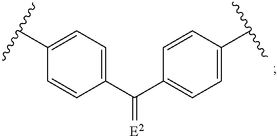
E² is O (oxygen) or N—OR^{D} where R^{D} in the definition of E² is selected from the group consisting of hydrogen and an optionally substituted C₁-C₆ alkyl;
each R¹¹ is separately selected from the group consisting of halogen, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₁-C₆ haloalkyl, and an optionally substituted C₁-C₆ heteroalkyl, an optionally substituted aryl, and an optionally substituted heteroaryl;
each R¹² is separately selected from the group consisting of —O(CH₂)ₘOR^{A}, —(CH₂)ₘOR^{A}, —NR^{B}R^{C}, and —(CH₂)ₘSR^{A};
each R¹³ is separately selected from the group consisting of —(CH₂)ₘOR^{D}, —NR^{E}R^{F}, —S(O)O₀₋₂R^{D}, —(CH₂)ₘNO₂, —(CH₂)ₘCN, and —(CH₂)ₘR^{G};
each R¹⁴ is separately selected from the group consisting of halogen, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl, an optionally substituted aryl, and an optionally substituted heteroaryl;
each R¹⁵ is separately selected from the group consisting of —O(CH₂)ₘOR^{A}, —(CH₂)ₘOR^{A}, —NR^{B}R^{C}, and —(CH₂)ₘSR^{A};
each R¹⁶ is separately selected from the group consisting of —(CH₂)ₘOR^{D}, —NR^{E}R^{F}, —(CH₂)ₘS(O)₀₋₂R^{D}, —(CH₂)ₘNO₂, —(CH₂)ₘCN, and —(CH₂)ₘR^{G};
E⁶ is CR¹⁷ when the dashed line between E⁶ and X represents a double bond; or E⁶ is CR¹⁷R¹⁷ when the dashed line between E⁶ and X represents a single bond;
F⁶ is CR¹⁸ when the dashed line between F⁶ and Y represents a double bond; or F⁶ is CR¹⁸R¹⁸ when the dashed line between F⁶ and Y represents a single bond;
each R¹⁷ is independently selected from the group consisting of hydrogen, halogen, an optionally substituted C₁-C₄ alkoxy, an optionally substituted C₃-C₇ cycloalkyl, and an optionally substituted C₁-C₄ alkyl;
each R¹⁸ is independently selected from the group consisting of hydrogen, halogen, an optionally substituted C₁-C₄ alkoxy, an optionally substituted C₃-C₇ cycloalkyl, and an optionally substituted C₁-C₄ alkyl;
R^{A} is selected from the group consisting of hydrogen, C₁-C₆ alkyl, C₂-C₄ alkenyl, C₂-C₄ alkynyl, C₃-C₇ cycloalkyl, C₁-C₆ haloalkyl, C₁-C₆ heteroalkyl, and C₁-C₆ heterohaloalkyl;
each —NR^{B}R^{C} is separately selected, wherein R^{B} and R^{C} are each independently selected from the group consisting of hydrogen, —SO₂R^{H}, —C(═O)R^{H}, —C(═O)NR^{E}R^{F}, heterocycle, C₁-C₆ alkyl, C₂-C₄ alkenyl, an optionally substituted C₂-C₄ alkynyl, C₃-C₇ cycloalkyl, C₁-C₆ haloalkyl, C₁-C₆ heteroalkyl, and C₁-C₆ heterohaloalkyl, where the C₁-C₆ alkyl in the definition of R^{B} and R^{C} is optionally substituted with an optionally substituted aryl or an optionally substituted heteroaryl and where the C₃-C₇ cycloalkyl and the heterocycle in the definition of R^{B} and R^{C} are optionally fused with an aryl or heteroaryl; or —NR^{B}R^{C} is an optionally substituted non-aromatic heterocycle linked through a ring nitrogen atom; or —NR^{B}R^{C} is an optionally substituted C₁-C₆ alkylideneamino;
each R^{D} is independently selected from the group consisting of hydrogen, an optionally substituted C₁-C₆ alkyl, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl, and —(CH₂)ₘR^{G};
each —NR^{E}R^{F} is separately selected, wherein R^{E} and R^{F} are each independently selected from the group consisting of hydrogen, an optionally substituted C₁-C₆ alkyl, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl, and —(CH₂)ₘR^{G}; or —NR^{E}R^{F} is an optionally substituted C₁-C₆ alkylideneamino; or —NR^{E}R^{F} is an optionally substituted non-aromatic heterocycle linked through a ring nitrogen atom; R^{G} is selected from an optionally substituted aryl and an optionally substituted heteroaryl;
R^{H} is selected from the group consisting of hydrogen, C₁-C₃ alkyl, an optionally substituted C₁-C₃ alkoxy, an optionally substituted C₂-C₄ alkenyl, an optionally substituted C₂-C₄ alkynyl, an optionally substituted C₃-C₇ cycloalkyl, C₂-C₄ alkenyl, C₂-C₄ alkynyl, C₃-C₇ cycloalkyl, C₁-C₃ haloalkyl, and an optionally substituted aryl or heteroaryl;
X and Y are independently selected from N (nitrogen), NH, CR¹⁹, and CR¹⁹R²⁰;
each R¹⁹ and R²⁰ are independently selected from the group consisting of hydrogen and an optionally substituted C₁-C₄ alkyl;
each m is independently 0, 1, or 2; and
any bond represented by a dashed and solid line represents a bond selected from the group consisting of a single bond and a double bond.

39. The method of claim 24, wherein the mimetic of EPO comprises a compound of Formula (III) having the structure: 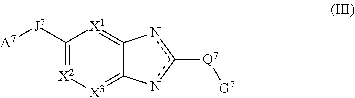
or a pharmaceutically acceptable salt, ester, stereoisomer, tautomer, or prodrug thereof;
wherein:
A⁷ is selected from the group consisting of aryl, heteroaryl, isoindolinyl, indenyl, dihydroindenyl, tetrahydroisoquinolinyl, and tetrahydronaphthalenyl, each optionally substituted with one or more substituents selected from the group consisting of R²¹, R²², and R²³, said aryl and heteroaryl in the definition of A⁷ are each further optionally fused with an optionally substituted nonaromatic heterocycle or an optionally substituted nonaromatic carbocycle; or A⁷ is C₃-C₇ cycloalkyl optionally substituted with one or more substituents selected from the group consisting of R²¹, R²², and R²³, said C₃-C₇ cycloalkyl in the definition of A⁷ is fused with an optionally substituted aryl or optionally substituted heteroaryl;
each R²¹ is independently selected from the group consisting of halogen, cyano, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted aryl, an optionally substituted heteroaryl, and an optionally substituted heterocycle;
each R²² is independently selected from the group consisting of —(CH₂)ₘOR^{A}, —O(CH₂)ₘOR^{A}, —C(═O)OR^{A}, —O(CH₂)ₒNR^{B}R^{C}, —(CH₂)ₘNR^{B}R^{C}, —C(═O)NR^{B}R^{C}, and —(CH₂)ₘSR^{A};
each R²³ is independently selected from the group consisting of phenyl, —NHC(═NH)NH₂, —(CH₂)ₘOR^{D}, —C(═NNR^{B}R^{C})H, —NR^{L}C(═O)NR^{B}R^{C}, —C(═O)NR^{D}N(═CHR^{G}), —(CH₂)ₘS(O)₀₋₂R^{D}, —(CH₂)ₘNO₂, —(CH₂)ₘCN, and —(CH₂)ₘR^{G}, said phenyl in the definition of R²³ is substituted with one or more substituents selected from the group consisting of halogen, cyano, C₁-C₃ alkyl, an optionally substituted C₁-C₃ alkoxy, —O(CH₂)ₘOR^{A}, —(CH₂)ₘNR^{B}R^{C};
J⁷ is selected from the group consisting of —(CH₂)ₙ[NHC(═O)](CH₂)ₒ[NHC(═O)](CH₂)ₚ—, —(CH₂)ₙ[NHC(═O)](CH₂)ₒ [NH]_{q}—, —NH[C(═O)][C(═O)]NH— and 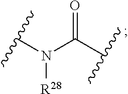
Q⁷ is selected from the group consisting of O (oxygen), —NR²⁸—, aryl, and arylamido; or Q⁷ is null;
each R²⁸ is independently selected from the group consisting of hydrogen and an optionally substituted C₁-C₄ alkyl;
G⁷ is selected from the group consisting aryl, heteroaryl, and heterocycle, each optionally substituted with one or more substituents selected from the group consisting of R²⁴, R²⁵, and R²⁶, said aryl and heteroaryl in the definition of G⁷ are each further optionally fused with an optionally substituted nonaromatic heterocycle or an optionally substituted nonaromatic carbocycle;
each R²⁴ is independently selected from the group consisting of halogen, cyano, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted aryl, an optionally substituted heteroaryl, and an optionally substituted heterocycle;
each R²⁵ is independently selected from the group consisting of —(CH₂)ₘOR^{A}, —O(CH₂)ₘOR^{A}, —C(═O)OR^{A}, —O(CH₂)ₒNR^{B}R^{C}, —(CH₂)ₘNR^{B}R^{C}, —(CH₂)ₘC(═O)NR^{B}R^{C}, —C(═NNR^{B}R^{C})H, and —(CH₂)ₘSR^{A};
each R²⁶ is independently selected from the group consisting of phenyl, —NHC(═NH)NH₂, —(CH₂)ₘOR^{D}, —C(═NNR^{B}R^{C})H, —NR^{L}C(═O)NR^{B}R^{C}, —C(═O)NR^{D}N(═CH)R^{G}, —(CH₂)ₘS(O)₀₋₂R^{D}, —(CH₂)ₘNO₂, —(CH₂)ₘCN, —(CH₂)ₘR^{G}, said phenyl in the definition of R²³ is substituted with —(CH₂)ₘNR^{B}R^{C};
X¹, X², and X³ are each independently selected from N (nitrogen) and CR²⁷;
R²⁷ is selected from the group consisting of hydrogen, halogen, and an optionally substituted C₁-C₄ alkyl;
R^{A} is selected from the group consisting of hydrogen, C₁-C₆ alkyl, C₂-C₄ alkenyl, C₂-C₄ alkynyl, C₃-C₇ cycloalkyl, and C₁-C₆ haloalkyl;
each —NR^{B}R^{C} is separately selected, wherein R^{B} and R^{C} are each independently selected from the group consisting of hydrogen, —SO₂R^{H}, —C(═O)R^{H}, —(CH₂)ₙOR^{H}, —(CH₂)ₘR^{I}, —(CH₂)ₘR^{J}, —(CH₂)ₙC(═O)NR^{E}R^{F}, —(CH₂)ₙNR^{E}R^{F}, —SO₂NR^{E}R^{F}, heterocycle, C₁-C₆ alkyl, C₁-C₆ haloalkyl, C₁-C₆ heteroalkyl, C₃-C₇ cycloalkyl, and C₁-C₆ heterohaloalkyl where the C₃-C₇ cycloalkyl and the heterocycle are each optionally fused with an optionally substituted aryl or optionally substituted heteroaryl; or —NR^{B}R^{C} is an optionally substituted non-aromatic heterocycle linked through a ring nitrogen atom, said optionally substituted non-aromatic heterocycle is optionally fused with an optionally substituted aryl or optionally substituted heteroaryl;
each R^{D} is independently selected from the group consisting of hydrogen, an optionally substituted C₁-C₆ alkyl, an optionally substituted C₁-C₆ haloalkyl, an optionally substituted C₁-C₆ heteroalkyl, and —(CH₂)ₘR^{I};
each —NR^{E}R^{F} is separately selected, wherein R^{E} and R^{F} are each independently selected from the group consisting of hydrogen, an optionally substituted C₁-C₆ alkyl, an optionally substituted C₁-C₆ haloalkyl, an optionally substituted C₁-C₆ heteroalkyl, an optionally substituted C₃-C₇ cycloalkyl, an optionally substituted heterocycle, and —(CH₂)ₘR^{G}; or —NR^{E}R^{F} is an optionally substituted non-aromatic heterocycle linked through a ring nitrogen atom; or —NR^{E}R^{F} is C₁-C₆ alkylideneamino substituted with an optionally substituted aryl;
each R^{G} is independently selected from an optionally substituted aryl and an optionally substituted heteroaryl;
each R^{H} is independently selected from the group consisting of hydrogen, C₁-C₃ alkoxy, C₁-C₃ alkyl, C₁-C₃ haloalkyl, an optionally substituted aryl and an optionally substituted heteroaryl;
each R^{I} is independently selected from the group consisting of an optionally substituted aryl and an optionally substituted heteroaryl;
each R^{J} is independently selected from the group consisting of aryl and heteroaryl, each substituted with one or more —NR^{E}R^{F};
each R^{L} is independently selected from the group consisting of C₃-C₇ cycloalkyl, optionally substituted C₁-C₆ alkyl, optionally substituted C₁-C₆ alkoxy, —(CH₂)ₘOR^{LA}, —(CH₂)ₘNR^{LB}R^{LC}, aryl and heteroaryl, said aryl and heteroaryl in the definition of R^{L} are each independently optionally substituted with one or more substituents selected from the group consisting of halo, C₁-C₆ alkyl, C₁-C₆ haloalkyl, C₁-C₆ alkoxy, —(CH₂)ₘNR^{LD}R^{LE}, aryl and heteroaryl, said aryl and heteroaryl substituent off of R^{L} are each optionally substituted with one or more halo, C₁-C₆ alkyl, C₁-C₆ haloalkyl, C₁-C₆ alkoxy, or —(CH₂)ₘNR^{LF}R^{LG};
each R^{LA} is independently selected from the group consisting of hydrogen, C₁-C₆ alkyl, and C₁-C₆ haloalkyl;
R^{LB} and R^{LC} are each independently selected from the group consisting of hydrogen, C₁-C₆ alkyl, C₁-C₆ haloalkyl, and C₁-C₆ heteroalkenyl; or —NR^{LB}R^{LC} is an optionally substituted non-aromatic heterocycle linked through a ring nitrogen atom;
each —NR^{LD}R^{LE} is separately selected, wherein R^{LD} and R^{LE} are each independently selected from the group consisting of hydrogen, aryl, heteroaryl, and optionally substituted C₁-C₆ alkyl, said aryl and heteroaryl in the definition of R^{LD} and R^{LE} are each optionally substituted with C₁-C₆ alkyl or C₁-C₆ alkoxy; or —NR^{LD}R^{LE} is an optionally substituted non-aromatic heterocycle linked through a ring nitrogen atom;
each —NR^{LF}R^{LG} is separately selected, wherein R^{LF} and R^{LG} are each independently selected from the group consisting of hydrogen, and C₁-C₆ alkyl; or —NR^{LF}R^{LG} is an optionally substituted non-aromatic heterocycle linked through a ring nitrogen atom;
each m is independently 0, 1, or 2;
each n is independently 0, 1, 2, 3, or 4;
each o is independently 1, 2, or 3;
each p is independently 0, 1, 2, or 3;
each q is independently 0 or 1; and
any bond represented by a dashed and solid line represents a bond selected from the group consisting of a single bond and a double bond.

40. The method of claim 24, wherein the mimetic of EPO comprises a compound of Formula (IV) having the structure: 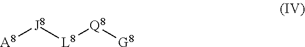
or a pharmaceutically acceptable salt, ester, stereoisomer, tautomer, or prodrug thereof;
wherein:
A⁸ is selected from the group consisting of heterocycle, aryl and heteroaryl, each optionally substituted with one or more substituents selected from the group consisting of R³¹, R³², and R³³, said aryl and heteroaryl in the definition of A⁸ are each further optionally fused with an optionally substituted nonaromatic heterocycle or an optionally substituted nonaromatic carbocycle;
G⁸ is selected from the group consisting of aryl and heteroaryl, each optionally substituted with one or more substituents selected from the group consisting of R³⁴, R³⁵, and R³⁶, said aryl and heteroaryl in the definition of G⁸ are each further optionally fused with an optionally substituted nonaromatic heterocycle or an optionally substituted nonaromatic carbocycle;
J⁸ is selected from the group consisting of aryl, heteroaryl, heterocycle, —C(═O)—, —(CH═CH)—, —OC(═O)—, —NHC(═O)NH—, —NHC(═S)NH—, —S(═O)₂—NH₂—, —OC(═S)—, —NHC(═S)—, —(CH₂)ₙNH—, —(CH₂)ₙ[NHC(═O)](CH₂)ₒNHC(═O)(CH₂)ₚ—, —(CH₂)ₙ[NHC(═O)](CH₂)ₒ[NH]_{q}—, 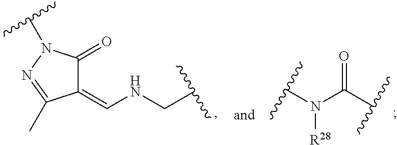
or J is null;
Q⁸ is selected from the group consisting of aryl, heteroaryl, heterocycle, —C(═O)—, —(CH═CH)—, —OC(═O)—, —NHC(═O)NH—, —NHC(═S)NH—, —S(═O)₂—N₂—, —OC(═S)—, —NHC(═S)—, —(CH₂)ₙNH—, —(CH₂)ₙ[NHC(═O)](CH₂)ₒNHC(═O)(CH₂)ₚ—, —(CH₂)ₙ[NHC(═O)](CH₂)ₒ[NH]_{q}—, 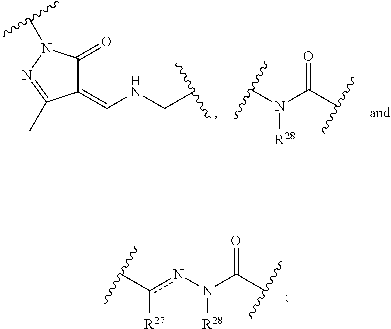
or Q⁸ is null;
L⁸ is selected from the group consisting of 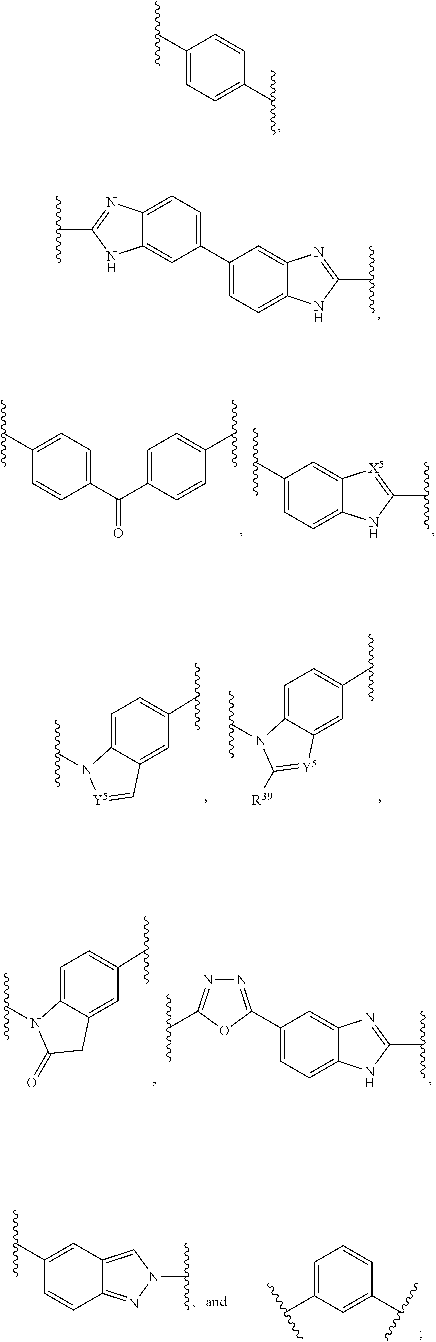
X⁵ is selected from the group consisting of N (nitrogen) and CR³⁹;
Y⁵ is selected from the group consisting of N (nitrogen) and CR⁴⁰;
each R²⁷ is independently selected from the group consisting of hydrogen, halogen, and an optionally substituted C₁-C₄ alkyl;
each R²⁸ is independently selected from the group consisting of hydrogen and an optionally substituted C₁-C₄ alkyl;
each R³¹ is independently selected from the group consisting of halogen, cyano, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₁-C₆ haloalkyl, and an optionally substituted C₁-C₆ heteroalkyl;
each R³² is independently selected from the group consisting of halogen, —(CH₂)ₘOR^{A}, —NR^{B}R^{C}, and —(CH₂)ₘSR^{A};
each R³³ is independently selected from the group consisting of halogen, —C(═O)OH, —(CH₂)ₘOR^{D}, —NR^{E}R^{F}, —NR^{L}C(═O)NR^{B}R^{C}, —(CH₂)ₘS(O)₀₋₂R^{D}, —(CH₂)ₘNO₂, —(CH₂)ₘCN, and —(CH₂)ₘR^{G};
each R³⁴ is independently selected from the group consisting of halogen, cyano, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₁-C₆ haloalkyl, and an optionally substituted C₁-C₆ heteroalkyl;
each R³⁵ is independently selected from the group consisting of halogen, C(═O)OH, —(CH₂)ₘOR^{A}, —NR^{B}R^{C}, —C(═O)NR^{B}R^{C}, and —(CH₂)ₘSR^{A};
each R³⁶ is independently selected from the group consisting of halogen, —(CH₂)ₘOR^{D}, —NR^{E}R^{F}, —NR^{L}C(═O)NR^{B}R^{C}, —(CH₂)ₘS(O)₀₋₂R^{D}, —(CH₂)ₘNO₂, —(CH₂)ₘCN, and —(CH₂)ₘR^{G};
each R³⁹ and R⁴⁰ are independently selected from the group consisting of hydrogen, halogen, —OH, —NHR^{B}, and an optionally substituted C₁-C₄ alkyl;
each R^{A} is independently selected from the group consisting of hydrogen, C₁-C₆ alkyl, C₁-C₆ haloalkyl, C₁-C₆ heteroalkyl, and C₁-C₆ heterohaloalkyl;
each —NR^{B}R^{C} is separately selected, wherein R^{B} and R^{C} are each independently selected from the group consisting of hydrogen, —SO₂R^{H}, —C(═O)R^{H}, —C(═O)C(═O)R^{H}, —(CH₂)ₘC(═O)OR^{H}, —C(═O)NR^{E}R^{F}, —(CH₂)ₘR^{G}, —(CH₂)ₘOR^{H}, —(CH₂)ₘR^{H}, C₁-C₆ alkyl, C₃-C₇ cycloalkyl, C₂-C₆ alkenyl, non-aromatic heterocycle, C₁-C₆ haloalkyl, C₁-C₆ heteroalkyl, and C₁-C₆ heterohaloalkyl where the C₃-C₇ cycloalkyl and the non-aromatic heterocycle are optionally fused with an an optionally substituted aryl or an optionally substituted heteroaryl; or —NR^{B}R^{C} is an optionally substituted non-aromatic heterocycle linked through a ring nitrogen atom; or —NR^{B}R^{C} is an optionally substituted C₁-C₆ alkylideneamino;
each R^{D} is independently selected from the group consisting of hydrogen, an optionally substituted C₁-C₆ alkyl, an optionally substituted C₁-C₆ haloalkyl, an optionally substituted C₁-C₆ heteroalkyl, and —(CH₂)ₘR^{G}
each —NR^{E}R^{F} is separately selected, wherein R^{E} and R^{F} are each independently selected from the group consisting of hydrogen, an optionally substituted C₁-C₆ alkyl, an optionally substituted C₁-C₆ haloalkyl, an optionally substituted C₁-C₆ heteroalkyl, and —(CH₂)ₘR^{G}; or —NR^{E}R^{F} is an optionally substituted C₁-C₆ alkylideneaminyl; or —NR^{E}R^{F} is an optionally substituted non-aromatic heterocycle linked through a ring nitrogen atom;
each R^{G} is independently selected from an optionally substituted aryl and an optionally substituted heteroaryl;
R^{H} is selected from the group consisting of hydrogen, C₁-C₃ alkyl, C₁-C₃ haloalkyl, C₃-C₇ cycloalkyl, and an optionally substituted aryl or an optionally substituted heteroaryl;
each R^{L} is independently selected from the group consisting of C₃-C₇ cycloalkyl, optionally substituted C₁-C₆ alkyl, optionally substituted C₁-C₆ alkoxy, —(CH₂)ₘOR^{LA}, —(CH₂)ₘNR^{LB}R^{LC}, aryl and heteroaryl, said aryl and heteroaryl in the definition of R^{L} are each independently optionally substituted with one or more substituents selected from the group consisting of halo, C₁-C₆ alkyl, C₁-C₆ haloalkyl, C₁-C₆ alkoxy, —(CH₂)ₘNR^{LD}R^{LE}, aryl and heteroaryl, said aryl and heteroaryl substituent off of R^{L} are each optionally substituted with one or more halo, C₁-C₆ alkyl, C₁-C₆ haloalkyl, C₁-C₆ alkoxy, or —(CH₂)ₘNR^{LF}R^{LG};
each R^{LA} is independently selected from the group consisting of hydrogen, C₁-C₆ alkyl, and C₁-C₆ haloalkyl;
R^{LB} and R^{LC} are each independently selected from the group consisting of hydrogen, C₁-C₆ alkyl, C₁-C₆ haloalkyl, and C₁-C₆ heteroalkenyl; or —NR^{LB}R^{LC} is an optionally substituted non-aromatic heterocycle linked through a ring nitrogen atom;
each —NR^{LD}R^{LE} is separately selected, wherein R^{LD} and R^{LE} are each independently selected from the group consisting of hydrogen, aryl, heteroaryl, and optionally substituted C₁-C₆ alkyl, said aryl and heteroaryl in the definition of R^{LD} and R^{LE} are each optionally substituted with C₁-C₆ alkyl or C₁-C₆ alkoxy; or —NR^{LD}R^{LE} is an optionally substituted non-aromatic heterocycle linked through a ring nitrogen atom;
each —NR^{LF}R^{LG} is separately selected, wherein R^{LF} and R^{LG} are each independently selected from the group consisting of hydrogen, and C₁-C₆ alkyl; or —NR^{LF}R^{LG} is an optionally substituted non-aromatic heterocycle linked through a ring nitrogen atom;
each —NR^{LF}R^{LG} is separately selected, wherein R^{LF} and R^{LG} are each independently selected from the group consisting of hydrogen, and C₁-C₆ alkyl; or —NR^{LF}R^{LG} is an optionally substituted non-aromatic heterocycle linked through a ring nitrogen atom;
each m is independently 0, 1, or 2;
each n is independently 0, 1, 2, 3, or 4;
each o is independently 1, 2, or 3;
each p is independently 0, 1, 2, or 3;
each q is independently 0 or 1; and
any bond represented by a dashed and solid line represents a bond selected from the group consisting of a single bond and a double bond.

41. The method of claim 24, wherein the mimetic of EPO comprises a compound of Formula (V) having the structure: 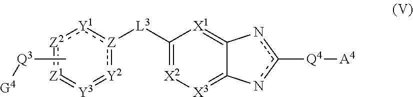
or a pharmaceutically acceptable salt, ester, stereoisomer, tautomer, or prodrug thereof;
wherein:
G⁴ is selected from the group consisting of is selected from the group consisting of aryl and heteroaryl, each optionally substituted with one or more substituents selected from the group consisting of R⁴³ and R⁴⁴, said aryl and heteroaryl in the definition of G⁴ are each further optionally fused with an optionally substituted nonaromatic heterocycle or an optionally substituted nonaromatic carbocycle;
Q³ is selected from the group consisting of an ester, an amide, a urea, a carbamide, a thioamide, a sulfonamide; or Q³ is selected from the group consisting of —C(═O)O—, —C(═O)NR⁴⁵—, —C(═O)NHN═CH—, —NR⁴⁵C(═O)NR⁴⁵—, —NR⁴⁵C(═O)(CH₂)ₘ₀—, —OC(═O)NR⁴⁵—, —C(═S)NR⁴⁵—, —NR⁴⁵S(O)₁₋₂—, C₁-C₆ alkylideneamino, and 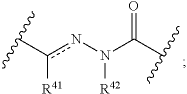
or when Y³ is C (carbon) substituted with -Q³-G⁴ then Q³ is optionally fused with Z¹ to form a five-member ring heterocycle;
L³ is selected from the group consisting of —C(═O)NR⁴⁵—, —O—C₁-C₈-alkyl, —C(═NR⁴⁵)—, —NR⁴⁵C(═O)—(CH₂)ₘC(═O)NR⁴⁵—, and —NR⁴⁵C(═O)—(CH₂)ₘNR⁴⁵C(═O)—;
Q⁴ is selected from the group consisting of NR⁴⁸, and O (oxygen); or Q⁴ is null;
A⁴ is selected from the group consisting of C₁-C₆ alkyl, C₂-C₆ alkenyl, C₂-C₆ alkynyl, C₃-C₇ cycloalkyl, C₃-C₇ cycloalkenyl, a C₁-C₆ heteroalkyl, phenyl, pyridinyl, imidazolyl, and thienyl, each optionally substituted with one or more substituents selected from the group consisting of R⁴¹ and R⁴²;
X¹, X², and X³ are each independently selected from N (nitrogen) and CR⁴⁶;
Y¹, Y², and Y³ are each independently selected from N (nitrogen) and CR⁴⁷;
Z, Z¹, and Z² are each independently selected from C (carbon), CH, and N (nitrogen);
R⁴¹ is independently selected from the group consisting of halogen, cyano, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl;
each R⁴² is independently selected from the group consisting of —(CH₂)ₘOR^{A}, —O(CH₂)ₘOR^{A}, —NR^{B}R^{C}, —C(═O)NR^{B}R^{C}, —C(═NNR^{B}R^{C})H, —(CH₂)ₘSR^{A}, —(CH₂)ₘR^{K}, —O(CH₂)ₘR^{K};
or R⁴¹ and R⁴² are linked to form an optionally substituted ring;
each R⁴³ is independently selected from the group consisting of halogen, cyano, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl;
each R⁴⁴ is independently selected from the group consisting of —(CH₂)ₘOR^{A}, —O(CH₂)ₘOR^{A}, —NR^{B}R^{C}, —C(═O)NR^{B}R^{C}, —C(═NNR^{B}R^{C})H, —(CH₂)ₘSR^{A}, —(CH₂)ₘR^{K}, —O(CH₂)ₘR^{K};
each R⁴⁵ is independently selected from the group consisting of hydrogen, an optionally substituted C₁-C₄ alkyl, and an optionally substituted C₁-C₆ heteroalkyl;
each R⁴⁶ and R⁴⁷ is independently selected from the group consisting of hydrogen, halogen, an optionally substituted C₁-C₆ alkyl, and an optionally substituted C₁-C₆ heteroalkyl;
R⁴⁸ is selected from the group consisting of hydrogen, an optionally substituted C₁-C₆ alkyl, an optionally substituted C₁-C₆ alkoxy, an optionally substituted C₂-C₄ alkenyl, an optionally substituted C₂-C₄ alkynyl, an optionally substituted C₃-C₇ cycloalkyl, an optionally substituted C₃-C₇ cycloalkenyl, and an optionally substituted C₁-C₆ heteroalkyl;
each R^{A} is independently selected from the group consisting of hydrogen, C₁-C₆ alkyl, C₁-C₆ haloalkyl, C₁-C₆ heteroalkyl, and C₁-C₆ heterohaloalkyl;
each —NR^{B}R^{C} is separately selected, wherein R^{B} and R^{C} are each independently selected from the group consisting of hydrogen, —SO₂R^{F}, —C(═O)R^{F}, —(CH₂)ₘR^{F}, —SO₂NR^{D}R^{E}, —C(═O)NR^{D}R^{E}, —(CH₂)ₘNR^{D}R^{E}, C₁-C₆ alkyl, C₃-C₇ cycloalkyl, C₃-C₇ cycloalkenyl, C₁-C₆ haloalkyl, C₁-C₆ heteroalkyl, and C₁-C₆ heterohaloalkyl where the alkyl and the heteroalkyl are optionally fused with an aryl or heteroaryl; or —NR^{B}R^{C} is an optionally substituted non-aromatic heterocycle linked through a ring nitrogen atom; or —NR^{B}R^{C} is an optionally substituted C₁-C₆ alkylideneamino;
each —NR^{D}R^{E} is separately selected, wherein R^{D} and R^{E} are each independently selected from the group consisting of hydrogen, an optionally substituted C₁-C₆ alkyl, an optionally substituted C₃-C₇ cycloalkyl, an optionally substituted C₃-C₇ cycloalkenyl, an optionally substituted C₁-C₆ haloalkyl, an optionally substituted C₁-C₆ heteroalkyl, and —(CH₂)ₘR^{G}; or —NR^{D}R^{E} is an optionally substituted C₁-C₆ alkylideneaminyl; or —NR^{D}R^{E} is an optionally substituted non-aromatic heterocycle linked through a ring nitrogen atom;
each R^{F} is independently selected from the group consisting of hydrogen, an optionally substituted C₁-C₄ alkyl, an optionally substituted C₁-C₄ haloalkyl, an optionally substituted C₃-C₇ cycloalkyl, an optionally substituted C₃-C₇ cycloalkenyl, aryl and heteroaryl, where the aryl and heteroaryl in the definition of R^{F} are each optionally substituted with —C(═O)NR^{D}R^{E} or —NR^{D}R^{E};
each R^{G} is independently selected from the group consisting of an optionally substituted aryl and an optionally substituted heteroaryl;
each R^{K} is independently selected from the group consisting of —C(═O)NR^{D}R^{E}, —NR^{D}R^{E}, an optionally substituted aryl and an optionally substituted heteroaryl;
each m is independently 0, 1, or 2; and
each dashed line represents an optional double bond.

42. The method of claim 24, wherein the mimetic of EPO comprises a compound of Formula (VI) having the structure: 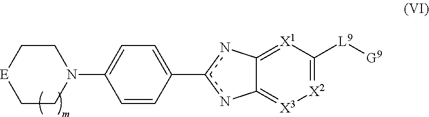
or a pharmaceutically acceptable salt, ester, stereoisomer, tautomer, or prodrug thereof;
wherein:
E is selected from the group consisting of O (oxygen), S (sulfur), NR⁴¹ and CR⁴²R⁴³;
R⁴¹ is selected from the group consisting of hydrogen, halogen, cyano, —C(═O)R^{C}, C₁-C₆ haloalkyl, C₁-C₆ heteroalkyl, and an optionally substituted C₁-C₆ alkyl;
R⁴² and R⁴³ are each independently selected from the group consisting of hydrogen, halogen, OR^{AA}, —OR^{C}c, —NR^{A}R^{B}, —NR^{C}R^{D}, SR^{AA}, —(CH₂)ₘR^{E}, —CONR^{C}R^{D}, an optionally substituted C₁-C₆ alkyl, an optionally substituted C₁-C₆ haloalkyl, and an optionally substituted C₁-C₆ heteroalkyl; or CR⁴²R⁴³ is an optionally substituted C₃-C₇ cycloalkyl;
X¹, X², and X³ are each independently selected from the group consisting of N (nitrogen) and CR⁴¹;
G⁹ is selected from the group consisting of aryl and heteroaryl, each optionally substituted with one or more substituents selected from the group consisting of R⁴⁴ and R⁴⁵, said aryl and heteroaryl in the definition of G⁹ are each further optionally fused with an optionally substituted nonaromatic heterocycle or an optionally substituted nonaromatic carbocycle;
each R⁴⁴ is separately selected from the group consisting of halogen, cyano, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted C₁-C₆ haloalkyl, and an optionally substituted C₁-C₆ heteroalkyl;
each R⁴⁵ is separately selected from the group consisting of hydrogen, halogen, —OR^{AA}, —OR^{C}c, —NR^{A}R^{B}, —NR^{C}R^{D}, —SR^{AA}, —(CH₂)ₘR^{E}, C₁-C₆ haloalkyl, C₁-C₆ heteroalkyl, and an optionally substituted C₁-C₆ alkyl;
each R^{AA} is independently selected from the group consisting of hydrogen, —(CH₂)ₘSO₂R^{F}, —(CH₂)ₘC(═O)RF, —(CH₂)ₘC(═O)NR^{C}R^{D}, an optionally substituted C₁-C₈ alkyl, an optionally substituted C₁-C₈ alkoxy, an optionally substituted C₂-C₈ alkenyl, an optionally substituted C₂-C₈ alkynyl, and an optionally substituted C₃-C₇ cycloalkyl, where said C₃-C₇ cycloalkyl is optionally fused with an aryl or heteroaryl;
each R^{BB} is independently selected from the group consisting of hydrogen, an optionally substituted C₁-C₈ alkyl, an optionally substituted C₁-C₈ alkoxy, an optionally substituted C₂-C₈ alkenyl, an optionally substituted C₂-C₈ alkynyl, an optionally substituted C₃-C₇ cycloalkyl, and (CH₂)ₘR^{E};
each —NR^{A}R^{B} is separately selected, wherein R^{A} and R^{B} are each independently selected from the group consisting of hydrogen, —(CH₂)ₘSO₂R^{F}, —(CH₂)ₘC(═O)R^{F}, —(CH₂)ₘC(═O)NR^{C}R^{D}, an optionally substituted C₁-C₈ alkyl, an optionally substituted C₁-C₈ alkoxy, an optionally substituted C₂-C₈ alkenyl, an optionally substituted C₂-C₈ alkynyl, and an optionally substituted C₃-C₇ cycloalkyl, where said C₃-C₇ cycloalkyl is optionally fused with an aryl or heteroaryl; or —NR^{A}R^{B} is an optionally substituted non-aromatic heterocycle linked through a ring nitrogen atom optionally fused with an aryl or heteroaryl; or —NR^{A}R^{B} is an optionally substituted C₁-C₆ alkylideneamino;
each —NR^{C}R^{D} is separately selected, wherein R^{C} and R^{D} are each independently selected from the group consisting of hydrogen, an optionally substituted C₁-C₈ alkyl, an optionally substituted C₁-C₈ alkoxy, an optionally substituted C₂-C₈ alkenyl, an optionally substituted C₂-C₈ alkynyl, an optionally substituted C₃-C₇ cycloalkyl, and (CH₂)ₘR^{E}; or —NR^{C}R^{D} is an optionally substituted C₁-C₈ alkylideneamino; or —NR^{C}R^{D} is an optionally substituted non-aromatic heterocycle linked through a ring nitrogen atom;
each R^{E} is separately selected from the group consisting of an optionally substituted aryl and an optionally substituted heteroaryl;
each R^{F} is separately selected from the group consisting of hydrogen, a an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted aryl and an optionally substituted heteroaryl;
L⁹ is selected from the group consisting of —(CH₂)ₘC(═O)NR⁴⁶(CH₂)_{q}—, —(CH₂)ₘC(═O)NR⁴⁶(CH₂)_{q}C(═O)NR⁴⁶—, —S(O)₂NH—, O (oxygen), —NR⁴⁶—, —OC(═O)O—, —OC(═O)NH—, —NHC(═O)NH—, —NHC═SNH—, —C(═NR⁴⁶)—, —C(═OO)NR⁴⁶—, —C(═S)NR⁴⁶—; or L⁹ is null;
each R⁴⁶ is independently selected from the group consisting of hydrogen, C₁-C₆ haloalkyl, and an optionally substituted C₁-C₆ alkyl;
each m is independently 0, 1, or 2;
each q is independently 1, 2, 3, 4, 5, or 6; and
any bond represented by a dashed and solid line represents a bond selected from the group consisting of a single bond and a double bond.

43. The method of claim 24, wherein the mimetic of EPO comprises a compound of Formula (VII) having the structure: 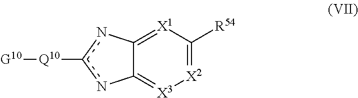
or a pharmaceutically acceptable salt, ester, stereoisomer, tautomer, or prodrug thereof;
wherein:
G¹⁰ is selected from the group consisting of C₁-C₆ alkyl, C₁-C₆ alkoxy, C₂-C₆ alkenyl, C₂-C₆ alkynyl, C₃-C₇ cycloalkyl, C₃-C₇ cycloalkenyl, C₁-C₆ haloalkyl, C₁-C₆ heteroalkyl, heterocycle, aryl and heteroaryl, each optionally substituted with one or more substituents selected from the group consisting of R⁵¹, R⁵², and R⁵³, said aryl and heteroaryl in the definition of G¹⁰ are each further optionally fused with an optionally substituted nonaromatic heterocycle or an optionally substituted nonaromatic carbocycle;
Q¹⁰ is selected from the group consisting of Q¹¹, Q¹¹-Q¹², and Q¹¹-Q¹²-Q¹³;
Q¹¹ and Q¹³ are each independently selected from the group consisting of piperazinyl, —C(═O)O—, —C(═O)NR⁵¹—, —NR⁵¹C(═O)NR⁵¹—, —OC(═O)NR⁵¹—, —C(═S)NR⁵¹—, —NR⁵¹S(O)₁₋₂—, —(CH₂)ₘC(═O)NR⁵¹(CH₂)_{q}—, and —(CH₂)ₘC(═O)NR⁵¹(CH₂)_{q}C(═O)NR⁵¹;
Q¹² is selected from the group consisting of an optionally substituted aryl, an optionally substituted heteroaryl and an optionally substituted heterocycle;
each R⁵¹ is separately selected from the group consisting of hydrogen, halogen, cyano, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted C₃-C₇ cycloalkenyl, an optionally substituted C₁-C₆ haloalkyl, and an optionally substituted C₁-C₆ heteroalkyl;
each R⁵² is separately selected from the group consisting of —(CH₂)ₘOR^{A}, —(CH₂)ₘNR^{B}R^{C}, —(CH₂)ₘSO₂NR^{B}R^{C}, and —(CH₂)ₘSR^{A};
each R⁵³ is separately selected from the group consisting of —(CH₂)ₘOR^{D}, —(CH₂)ₘNR^{E}R^{F}, —(CH₂)ₘS(O)₀₋₂R^{D}, —(CH₂)ₘNO₂, —(CH₂)ₘCN, and —(CH₂)ₘR^{G};
each R⁵⁴ is separately selected from the group consisting of hydrogen, —(CH₂)ₘOR^{A}, —(CH₂)ₘNR^{B}R^{C}, —O(CH₂)ₘNR^{B}R^{C}, —C(═O)NR^{B}R^{C}, —(CH₂)ₘSR^{A}, —(CH₂)ₘR^{G}, —O(CH₂)ₘR^{G}, —(CH₂)ₘSO₂NR^{B}R^{C}, —(CH₂)ₘCN, an optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, an optionally substituted C₃-C₇ cycloalkenyl, an optionally substituted C₁-C₆ haloalkyl, and an optionally substituted C₁-C₆ heteroalkyl;
each R^{A} is separately selected from the group consisting of hydrogen, an optionally substituted C₁-C₆ alkyl, an optionally substituted C₂-C₆ alkenyl, an optionally substituted C₂-C₆ alkynyl, an optionally substituted C₃-C₇ cycloalkyl, an optionally substituted C₃-C₇ cycloalkenyl, an optionally substituted C₁-C₆ haloalkyl, and an optionally substituted C₁-C₆ heteroalkyl;
each —NR^{B}R^{C} is separately selected, wherein R^{B} and R^{C} are each independently selected from the group consisting of hydrogen, —(CH₂)ₘSO₂R^{H}, —(CH₂)ₘCOR^{H}, —(CH₂)ₘCONR^{E}R^{F}, an optionally substituted C₁-C₈ alkyl, an optionally substituted C₁-C₈ alkoxy, an optionally substituted C₂-C₈ alkenyl, an optionally substituted C₂-C₈ alkynyl, an optionally substituted C₃-C₇ cycloalkyl, and —(CH₂)ₘR^{G}, where said C₃-C₇ cycloalkyl is optionally fused with an aryl or heteroaryl; or —NR^{B}R^{C} or is an optionally substituted non-aromatic heterocycle linked through a ring nitrogen atom optionally fused with an aryl or heteroaryl; or —NR^{B}R^{C} is an optionally substituted C₁-C₈ alkylideneamino;
each R^{D} is separately selected from the group consisting of hydrogen, an optionally substituted C₁-C₈ alkyl, an optionally substituted C₂-C₈ alkenyl, an optionally substituted C₂-C₈ alkynyl, an optionally substituted C₃-C₈ cycloalkyl, an optionally substituted C₃-C₈ cycloalkenyl, an optionally substituted C₁-C₈ haloalkyl, and an optionally substituted C₁-C₈ heteroalkyl;
each —NR^{E}R^{F} is separately selected, wherein R^{E} and R^{F} are each independently selected from the group consisting of hydrogen, an optionally substituted C₁-C₈ alkyl, an optionally substituted C₁-C₈ alkoxy, an optionally substituted C₂-C₈ alkenyl, an optionally substituted C₂-C₈ alkynyl, an optionally substituted C₃-C₇ cycloalkyl, and (CH₂)ₘR^{G}; or —NR^{E}R^{F} or is an optionally substituted non-aromatic heterocycle linked through a ring nitrogen atom; or —NR^{C}R^{D} is an optionally substituted C₁-C₈ alkylideneamino;
each R^{G} is separately selected from a substituted or unsubstituted aryl and a substituted or unsubstituted heteroaryl;
each R^{H} is separately selected from the group consisting of hydrogen, a C₁-C₆ alkyl, C₂-C₆ alkenyl, C₂-C₆ alkynyl, C₃-C₇ cycloalkyl, C₃-C₇ cycloalkenyl, C₁-C₆ haloalkyl, C₁-C₆ heteroalkyl, an optionally substituted heterocycle, an optionally substituted aryl, and an optionally substituted heteroaryl;
X¹, X², and X³ are each independently selected from the group consisting of N (nitrogen) and CR⁴⁷;
each R⁴⁷ is separately selected from the group consisting of hydrogen, halogen, an optionally substituted C₁-C₆ alkyl, and an optionally substituted C₁-C₆ heteroalkyl
each m is independently 0, 1, 2, or 3; and
any bond represented by a dashed and solid line represents a bond selected from the group consisting of a single bond and a double bond.

44. The method of claim 24, wherein the mimetic of EPO comprises a compound of Formula (X) having the structure: 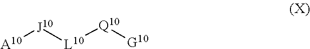
or a pharmaceutically acceptable salt, ester, stereoisomer, tautomer, or prodrug thereof;
wherein:
A¹⁰ is selected from the group consisting of C₁-C₆ alkyl, C₁-C₆ alkoxy, C₂-C₆ alkenyl, C₂-C₆ alkynyl, C₃-C₇ cycloalkyl, C₃-C₇ cycloalkenyl, C₁-C₆ haloalkyl, C₁-C₆ heteroalkyl, heterocycle, aryl, and heteroaryl, each optionally substituted with one or more substituents selected from the group consisting of R¹, R², and R³, said aryl and heteroaryl in the definition of A¹⁰ are each further optionally fused with an optionally substituted nonaromatic heterocycle or an optionally substituted nonaromatic carbocycle;
G¹⁰ is selected from the group consisting of C₁-C₆ alkyl, C₁-C₆ alkoxy, C₂-C₆ alkenyl, C₂-C₆ alkynyl, C₃-C₇ cycloalkyl, C₃-C₇ cycloalkenyl, C₁-C₆ haloalkyl, C₁-C₆ heteroalkyl, heterocycle, aryl, and heteroaryl, each optionally substituted with one or more substituents selected from the group consisting of R⁴, R⁵, and R⁶, said aryl and heteroaryl in the definition of G¹⁰ are each further optionally fused with an optionally substituted nonaromatic heterocycle or an optionally substituted nonaromatic carbocycle;
J¹⁰ is a 1-8 atom long spacer containing at least 2 heteroatoms separated by 2 bonds and comprising one or more groups selected from —S(O)₂NR^{A}—, an optionally substituted C₁-C₆ heteroalkyl, an optionally substituted heterocycle, and an optionally substituted heteroalkylheterocycle; including the proviso that J¹⁰ is not a 1-8 atom spacer containing at least 2 heteroatoms separated by 3 or 4 bonds and comprising one or more groups selected from an optionally substituted C₁-C₆ heteroalkyl, an optionally substituted heterocycle, and an optionally substituted heteroalkylheterocycle;
Q¹⁰ is a 1-8 atom long spacer containing at least 2 heteroatoms separated by 2 bonds and comprising one or more groups selected from —S(O)₂NR^{A}—, an optionally substituted C₁-C₆ heteroalkyl, an optionally substituted heterocycle, and an optionally substituted heteroalkylheterocycle;
L¹⁰ is a 2-14 atom long linker comprising one or more groups selected from —O— (oxygen), —C(═O)—, —C(═S)—, —NR^{A}—, —S(O)₀₋₂—, —NR^{A}S(O)₁₋₂NR^{A}—, and —NR^{A}S(O)₁₋₂O—, and one or more groups selected from —O— (oxygen), —C(═O)—, —C(═S)—, —NR^{A}, —S(O)₀₋₂—, —NR^{A}S(O)₁₋₂NR^{A}—, and —NR^{A}S(O)₁₋₂O—, an optionally substituted C₁-C₁₀ alkyl, an optionally substituted aryl, and an optionally substituted heteroaryl; where the an optionally substituted aryl and an optionally substituted heteroaryl in the definition of L¹⁰ are each further optionally fused with an optionally substituted nonaromatic heterocycle or an optionally substituted nonaromatic carbocycle;
R¹ is selected from the group consisting of halogen, optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, optionally substituted C₃-C₇ cycloalkenyl, and an optionally substituted C₁-C₆ heteroalkyl;
R² is selected from the group consisting of halogen, —OR^{A}, NR^{B}R^{C}, —SR^{A};
R³ is selected from the group consisting of —OR^{D}, NR^{E}R^{F}, —S(O)₀₋₂R^{D}, —NO₂, —CN, and —(CH₂)ₘR^{G},;
R⁴ is selected from the group consisting of halogen, optionally substituted C₁-C₆ alkyl, an optionally substituted C₁-C₆ alkoxy, an optionally substituted C₂-C₆ alkenyl, an optionally substituted C₂-C₆ alkynyl, an optionally substituted C₃-C₇ cycloalkyl, optionally substituted C₃-C₇ cycloalkenyl, an optionally substituted C₁-C₆ heteroalkyl;
R⁵ is selected from the group consisting of —OR^{A}, NR^{B}R^{C}, —SR^{A};
R⁶ is selected from the group consisting of —OR^{D}, NR^{E}R^{F}, —S(O)₀₋₂R^{D}, —NO₂, —CN, and —(CH₂)ₘR^{G};
each R^{A} is separately selected from the group consisting of hydrogen, —SO₂R^{F}, —C(═O)R^{F}, —C(═O)NR^{C}R^{D}, C₁-C₆ alkyl, C₂-C₆ alkenyl, C₂-C₆ alkynyl, C₃-C₇ cycloalkyl, C₃-C₇ cycloalkenyl, C₁-C₆ haloalkyl, C₁-C₆ heteroalkyl, and C₁-C₆ heterohaloalkyl, where the C₁-C₆ alkyl, C₃-C₇ cycloalkyl, C₁-C₆ heteroalkyl, and C₁-C₆ heterohaloalkyl in the definition of R^{A} are optionally substituted;
each —NR^{B}R^{C} is separately selected, wherein R^{B} and R^{C} are each independently selected from the group consisting of hydrogen, —SO₂R^{H}, —C(═O)R^{H}, —C(═O)NR^{E}R^{F}, C₁-C₆ alkyl, C₂-C₄ alkenyl, an optionally substituted C₂-C₄ alkynyl, C₃-C₇ cycloalkyl, C₁-C₆ haloalkyl, C₁-C₆ heteroalkyl, heterocycle, and C₁-C₆ heterohaloalkyl where the cycloalkyl and the heterocycle are optionally fused with an aryl or heteroaryl; or —NR^{B}R^{C} is an optionally substituted non-aromatic heterocycle linked through a ring nitrogen atom; or —NR^{B}R^{C} is an optionally substituted C₁-C₆ alkylideneamino;
each R^{D} is independently selected from the group consisting of hydrogen, an optionally substituted C₁-C₆ alkyl, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl, an optionally substituted heterocyle, and —(CH₂)ₘR^{G};
each —NR^{E}R^{F} is separately selected, wherein R^{E} and R^{F} are each independently selected from the group consisting of hydrogen, an optionally substituted C₁-C₆ alkyl, an optionally substituted C₂-C₄ alkenyl, an optionally substituted C₂-C₄ alkynyl, an optionally substituted C₃-C₇ cycloalkyl, an optionally substituted C₁-C₆ haloalkyl, an optionally substituted C₁-C₆ heteroalkyl, an optionally substituted heterocycle, and —(CH₂)ₘR^{G}; or —NR^{E}R^{F} is an optionally substituted non-aromatic heterocycle linked through a ring nitrogen atom;
each R^{G} is separately selected from an optionally substituted aryl and an optionally substituted heteroaryl;
each R^{H} is separately selected from the group consisting of hydrogen, a C₁-C₆ alkyl, a C₁-C₆ haloalkyl, a C₁-C₆ heteroalkyl, a C₃-C₆ cycloalkyl, an optionally substituted heterocycle, and an optionally substituted aryl or an optionally substituted heteroaryl; and
each m is independently 0, 1, or 2.
